(19) United States
(12) Patent Application Publication
(10) Pub. No.: US 2026/0085067 A1
(43) Pub. Date: Mar. 26, 2026

(54) CYCLIC SULFONAMIDE RIBONUCLEOTIDE REDUCTASE (RNR) INHIBITORS AND USES THEREOF

(71) Applicant: Boundless Bio, Inc., San Diego, CA (US)

(72) Inventors: Anthony B. PINKERTON, Rancho Santa Fe, CA (US); Jacques MAUGER, San Diego, CA (US); Yen Pham Hong TRUONG, San Diego, CA (US)

(21) Appl. No.: 19/110,333

(22) PCT Filed: Sep. 12, 2023
(86) PCT No.: PCT/US2023/073976

§ 371 Date: Mar. 10, 2025

Related U.S. Application Data
(60) Provisional application No. 63/375,495, filed on Sep. 13, 2022.

(51) Int. Cl. C07D 417/06; A61K 31/5415; A61K 31/549; A61K 31/554; A61P 35/00; C07B 59/00; C07D 417/14; C07D 419/06; C07D 513/04; C07D 515/04

(52) U.S. Cl. CPC C07D 417/06; A61K 31/5415; A61K 31/549; A61K 31/554; A61P 35/00; C07B 59/002; C07D 417/14; C07D 419/06; C07D 513/04; C07D 515/04

(57) ABSTRACT

Provided herein are compounds and methods for the treatment of cancer. The methods include administering to a subject in need a therapeutically effective amount of a cyclic sulfonamide RNR inhibitor disclosed herein.

### CROSS-REFERENCE

This application claims the benefit of U.S. Provisional Application Ser. No. 63/375,495 filed Sep. 13, 2022 which is hereby incorporated by reference in its entirety.

### FIELD OF THE INVENTION

Described herein are compounds, methods of making such compounds, pharmaceutical compositions, and medicaments comprising such compounds, and methods of using such compounds for inhibiting ribonucleotide reductase (RNR).

### BACKGROUND OF THE INVENTION

Ribonucleotide reductase (RNR), also known as ribonucleotide diphosphate reductase (rNDP), is composed of a hetero-oligomer of a large subunit M1 and a small subunit M2, and expression of both is required for enzyme activity. RNR is a highly regulated enzyme in the deoxyribonucleotide synthesis pathway that is ubiquitously present in human, bacteria, yeast, and other organisms. RNR is responsible for the de novo conversion of ribonucleotide diphosphate to 2′-deoxyribonucleotide diphosphate, a process that is essential for DNA synthesis and repair. RNR is directly involved in DNA synthesis and repair, tumor growth, metastasis, and drug resistance. In various types of solid tumors and blood cancers, numerous correlations have been reported with overexpression of M2 and their prognosis. In addition, cell growth inhibition by inhibiting RNR and anti-tumor effect in vivo have been reported in cell lines derived from several cancer types and in nonclinical models.

The proliferation of cancer cells requires excess deoxyribonucleotide triphosphates (dNTPs) for DNA synthesis. Therefore, an increase in RNR activity is necessary as it helps provide extra dNTPs for DNA replication in primary and metastatic cancer cells. Because of this critical role in DNA synthesis, RNR represents an important target for cancer therapy. However, existing chemotherapies that target RNR are nucleoside-based analogs. Hence, they are promiscuous, leading to nonspecific binding of other nucleoside binding proteins which results in unwanted side effects. Therefore, there is a need for compositions and methods for specifically targeting and inhibiting RNR activity in neoplastic cells in the treatment of cancer.

### BRIEF SUMMARY OF THE INVENTION

Described herein are RNR inhibitors that are useful in treating cancer.

Disclosed herein is a compound of Formula (I), or a pharmaceutically acceptable salt, solvate, tautomer, or stereoisomer thereof:

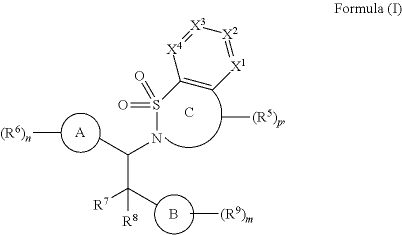

Also disclosed herein is a pharmaceutical composition comprising a compound disclosed herein, or a pharmaceutically acceptable salt, solvate, tautomer, or stereoisomer thereof, and a pharmaceutically acceptable excipient.

Also disclosed herein is a method of treating cancer in a subject, comprising administering to the subject a compound disclosed herein, or a pharmaceutically acceptable salt, solvate, tautomer, or stereoisomer thereof, or a pharmaceutical composition disclosed herein.

Also disclosed herein is a method of inhibiting ribonucleotide reductase in a subject, comprising administering to the subject a compound disclosed herein, or a pharmaceutically acceptable salt, solvate, tautomer, or stereoisomer thereof, or a pharmaceutical composition disclosed herein.

In some embodiments, the inhibition of ribonucleotide reductase occurs in a tumor cell in the subject in need thereof.

Also disclosed herein is a method for treating a tumor or tumor cells in a subject, the method comprising administering a compound disclosed herein, or a pharmaceutically acceptable salt, solvate, tautomer, or stereoisomer thereof, or the pharmaceutical composition disclosed herein, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, tautomer, or stereoisomer thereof, or the pharmaceutical composition disclosed herein, to a subject identified as having a tumor or tumor cells having ecDNA, wherein growth or size of the tumor or growth or number of the tumor cells is decreased as a result of treatment. In some embodiments, the method further comprises administering a cancer-targeted therapeutic agent. In some embodiments, the cancer-targeted therapeutic agent inhibits a gene or gene product comprised on ecDNA in the tumor or tumor cells.

Also disclosed herein is a method for treating a tumor or tumor cells in a subject, the method comprising administering a compound disclosed herein, or a pharmaceutically acceptable salt, solvate, tautomer, or stereoisomer thereof, or the pharmaceutical composition disclosed herein, in an amount sufficient to induce replication stress in the tumor or tumor cells, wherein the tumor or tumor cells comprises ecDNA or have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, tautomer, or stereoisomer thereof, or the pharmaceutical composition disclosed herein, to a subject identified as having a tumor or tumor cells having a focal amplification of an oncogene, wherein growth or size of the tumor or growth or number of the tumor cells is decreased as a result of treatment. In some embodiments, the method further comprises administering a cancer-targeted therapeutic agent, wherein the target of the therapeutic agent is a protein encoded by the oncogene. In some embodiments, the focal amplification is present on ecDNA.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Amine” refers to —NH₂;

“hydroxy” refers to —OH;

“Carboxyl” refers to —COOH.

“Alkyl” refers to a straight-chain or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl. In some embodiments, the alkyl is a C₁-C₆ alkyl. In some embodiments, the alkyl is a C₁-C₅ alkyl. In some embodiments, the alkyl is a C₁-C₄ alkyl. In some embodiments, the alkyl is a C₁-C₃ alkyl. Unless stated otherwise specifically in the specification, an alkyl group may be optionally substituted, for example, with one or more oxo, halogen, amino, nitrile, nitro, hydroxyl, haloalkyl, alkoxy, carboxyl, carboxylate, aryl, cycloalkyl, heterocycloalkyl, or heteroaryl, and the like. In some embodiments, the alkyl is optionally substituted with one or more oxo, halogen, —CN, —COOH, —COOMe, —OH, —OMe, —NH₂, or —NO₂. In some embodiments, the alkyl is optionally substituted with one or more halogen, —CN, —OH, or —OMe. In some embodiments, the alkyl is optionally substituted with halogen.

“Alkenyl” refers to a straight-chain or branched-chain hydrocarbon monoradical having one or more carbon-carbon double-bonds and having from two to about ten carbon atoms, more preferably two to about six carbon atoms. The group may be in either the cis or trans or Z or E conformation about the double bond(s), and should be understood to include all isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with one or more oxo, halogen, amino, nitrile, nitro, hydroxyl, alkyl, haloalkyl, alkoxy, carboxyl, carboxylate, aryl, cycloalkyl, heterocycloalkyl, or heteroaryl, and the like. In some embodiments, the alkenyl is optionally substituted with one or more oxo, halogen, —CN, —COOH, —COOMe, —OH, —OMe, —NH₂, or —NO₂. In some embodiments, the alkenyl is optionally substituted with one or more halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with one or more oxo, halogen, amino, nitrile, nitro, hydroxyl, alkyl, haloalkyl, alkoxy, carboxyl, carboxylate, aryl, cycloalkyl, heterocycloalkyl, or heteroaryl, and the like. In some embodiments, the alkynyl is optionally substituted with one or more oxo, halogen, —CN, —COOH, —COOMe, —OH, —OMe, —NH₂, or —NO₂. In some embodiments, the alkynyl is optionally substituted with one or more halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with one or more oxo, halogen, amino, nitrile, nitro, hydroxyl, alkyl, haloalkyl, alkoxy, carboxyl, carboxylate, aryl, cycloalkyl, heterocycloalkyl, or heteroaryl, and the like. In some embodiments, the alkylene is optionally substituted with one or more oxo, halogen, —CN, —COOH, —COOMe, —OH, —OMe, —NH₂, or —NO₂. In some embodiments, the alkylene is optionally substituted with one or more halogen, —CN, —OH, or —OMe. In some embodiments, the alkylene is optionally substituted with halogen.

“Alkoxy” refers to a radical of the formula —Oalkyl where alkyl is defined as above. Unless stated otherwise specifically in the specification, an alkoxy group may be optionally substituted, for example, with one or more oxo, halogen, amino, nitrile, nitro, hydroxyl, alkyl, haloalkyl, alkoxy, carboxyl, carboxylate, aryl, cycloalkyl, heterocycloalkyl, or heteroaryl, and the like. In some embodiments, the alkoxy is optionally substituted with one or more halogen, —CN, —COOH, —COOMe, —OH, —OMe, —NH₂, or —NO₂. In some embodiments, the alkoxy is optionally substituted with one or more halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to anthracenyl, naphthyl, phenanthrenyl, azulenyl, phenyl, chrysenyl, fluoranthenyl, fluorenyl, as-indacenyl, s-indacenyl, indanyl, indenyl, phenalenyl, phenanthrenyl, pleiadenyl, pyrenyl, and triphenylenyl. Unless stated otherwise specifically in the specification, an aryl may be optionally substituted, for example, with one or more halogen, amino, nitrile, nitro, hydroxyl, alkyl, alkenyl, alkynyl, haloalkyl, alkoxy, carboxyl, carboxylate, aryl, cycloalkyl, heterocycloalkyl, or heteroaryl, and the like. In some embodiments, the aryl is optionally substituted with one or more halogen, methyl, ethyl, —CN, —COOH, —COOMe, —CF₃, —OH, —OMe, —NH₂, or —NO₂. In some embodiments, the aryl is optionally substituted with one or more halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, and/or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃—C cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octyl, bicyclo[4.3.0]nonyl, cis-decalinyl, trans-decalinyl, bicyclo[2.1.1]hexyl, bicyclo[2.2.1]heptyl, bicyclo[2.2.2]octyl, bicyclo[3.2.1]octyl, bicyclo[3.2.2]nonyl, and bicyclo[3.3.2]decyl, bicyclo[1.1.1]pentyl, bicyclo[3.1.0]hexyl, bicyclo[3.1.1]heptyl, 7,7-dimethyl-bicyclo[2.2.1]heptanyl, spiro[4.2]heptyl, spiro[4.3]octyl, spiro[5.2]octyl, spiro[3.3]heptyl, and spiro[5.3]nonyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with one or more oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, or heteroaryl, and the like. In some embodiments, a cycloalkyl is optionally substituted with one or more oxo, halogen, methyl, ethyl, —CN, —COOH, —COOMe, —CF₃, —OH, —OMe, —NH₂, or —NO₂. In some embodiments, a cycloalkyl is optionally substituted with one or more oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, bromomethyl, trichloromethyl, 2,2,2-trifluoroethyl, 1,2-difluoroethyl, 2,2-difluoroethyl, 2-fluoroethyl, 3-bromo-2-fluoropropyl, 1,2-dibromoethyl, 1-chloroethyl, and the like.

“Haloalkoxy” refers to —O-haloalkyl, with haloalkyl as defined above.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl includes, for example, hydroxymethyl, hydroxyethyl, hydroxypropyl, hydroxybutyl, or hydroxypentyl. In some embodiments, the hydroxyalkyl is hydroxymethyl. In some embodiments, the hydroxyalkyl is 1-hydroxyeth-1-yl, 2-hydroxy-prop-2-yl, 2-hydroxy-2-methylprop-1-yl, or 2,3-dihydroxypropyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl includes, for example, aminomethyl, aminoethyl, aminopropyl, aminobutyl, aminopentyl, CH₂N(CH₃)₂, or CH(CH₃)N(CH₃)₂. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen, sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen, sulfur, phosphorus, or combinations thereof wherein the heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or two atoms selected from the group consisting of oxygen, nitrogen, and sulfur wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂CH₂OCH₃, —CH₂CH₂OCH₂CH₂OCH₃, —CH(CH₃)OCH₃, —CH₂C(CH₃)₂OCH₃, —CH₂NHCH₃, —CH₂N(CH₃)₂, —CH(CH₃)N(CH₃)₂, —CH₂CH₂NHCH₃, or —CH₂CH₂N(CH₃)₂. Unless stated otherwise specifically in the specification, a heteroalkyl is optionally substituted for example, with one or more oxo, halogen, amino, nitrile, nitro, hydroxyl, alkyl, alkenyl, alkynyl, haloalkyl, alkoxy, aryl, cycloalkyl, heterocycloalkyl, or heteroaryl, and the like. In some embodiments, a heteroalkyl is optionally substituted with one or more oxo, halogen, methyl, ethyl, —CN, —CF₃, —OH, —OMe, —NH₂, or —NO₂. In some embodiments, a heteroalkyl is optionally substituted with one or more oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl is C-linked. In some embodiments, the heterocycloalkyl is N-linked. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. In some embodiments, the heterocycloalkyl comprises one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl is optionally substituted, for example, with one or more oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like.

In some embodiments, the heterocycloalkyl is optionally substituted with one or more oxo, halogen, methyl, ethyl, —CN, —COOH, —COOMe, —CF₃, —OH, —OMe, —NH₂, or —NO₂. In some embodiments, the heterocycloalkyl is optionally substituted with one or more halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. In some embodiments, the heteroaryl is C-linked. In some embodiments, the heteroaryl is N-linked. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl comprising 1, 2, or 3 heteroatoms selected from the group consisting of oxygen, nitrogen, and sulfur. In some embodiments, the heteroaryl is a 5- to 6-membered heteroaryl comprising 1, 2, or 3 heteroatoms selected from the group consisting of oxygen, nitrogen, and sulfur. In some embodiments, the heteroaryl is a 6-membered heteroaryl comprising 1, 2, or 3 heteroatoms selected from the group consisting of oxygen, nitrogen, and sulfur. In some embodiments, the heteroaryl is a 5-membered heteroaryl comprising 1, 2, or 3 heteroatoms selected from the group consisting of oxygen, nitrogen, and sulfur.

Examples include, but are not limited to, azepinyl, acridinyl, benzimidazolyl, benzothiazolyl, benzindolyl, benzodioxolyl, benzofuranyl, benzoxazolyl, benzothiadiazolyl, benzo[b][1,4]dioxepinyl, 1,4-benzodioxanyl, benzonaphthofuranyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, oxadiazolonyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with one or more halogen, amino, nitrile, nitro, hydroxyl, alkyl, alkenyl, alkynyl, haloalkyl, alkoxy, carboxyl, carboxylate, aryl, cycloalkyl, heterocycloalkyl, or heteroaryl, and the like. In some embodiments, the heteroaryl is optionally substituted with one or more halogen, methyl, ethyl, —CN, —COOH, —COOMe, —CF₃, —OH, —OMe, —NH₂, or —NO₂. In some embodiments, the heteroaryl is optionally substituted with one or more halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The term “one or more” when referring to an optional substituent means that the subject group is optionally substituted with one, two, three, or four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents. In some embodiments, the subject group is optionally substituted with three substituents.

The terms “treat,” “treated,” “treatment,” or “treating” as used herein refers to therapeutic treatment, wherein the object is to slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “ecDNA signature” as used herein, generally refers to one or more characteristics common to tumors or tumor cells that are ecDNA+(contain extrachromosomal DNA (ecDNA)). In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, ecDNA signature includes a detection or identification of ecDNA using an imaging technology. In some cases, ecDNA signature does not include any imaging or direct detection of ecDNA.

### Compounds

Described herein are cyclic sulfonamide RNR inhibitors that are useful for the treatment of cancer.

Disclosed herein is a compound of Formula (I), or a pharmaceutically acceptable salt, solvate, tautomer, or stereoisomer thereof:

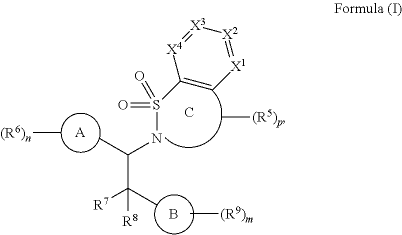

wherein:
X¹ is N or CR¹;
X² is N or CR²;
X³ is N or CR³;
X⁴ is N or CR⁴;
R¹ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R² is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R³ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R′, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R⁴ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
Ring C is a 5- to 8-membered heterocycloalkyl comprising one or two additional heteroatoms selected from the group consisting of —O—, —S—, —S(═O)—, —S(═O)₂—, and —NR¹⁰—;
R¹⁰ is hydrogen, —OH, —OR^{a}, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{10a};
each R^{10a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{10a} on the same carbon are taken together to form an oxo;
each R⁵ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁵ on the same carbon are taken together to form an oxo;
or two R⁵ on the same carbon are taken together to form a cycloalkyl or heterocycloalkyl; each optionally substituted with one or more R;
or two R⁵ on adjacent atoms are taken together to form a cycloalkyl, heterocycloalkyl, aryl, or heteroaryl; each optionally substituted with one or more R;
or one R⁵ and R¹⁰ are taken together to form a heterocycloalkyl or heteroaryl; each optionally substituted with one or more R;
p is 0-4;
Ring A is a 5-membered heterocycloalkyl or 5-membered heteroaryl;
each R⁶ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁶ on the same atom are taken together to form an oxo;
n is 0-3;
R⁷ is hydrogen, deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
R⁸ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
Ring B is cycloalkyl, heterocycloalkyl, aryl, or heteroaryl;
each R⁹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R⁹;
or two R⁹ on the same atom are taken together to form an oxo;
each R^{9a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{9a} on the same atom are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, cycloalkyl, or heterocycloalkyl;
or two R on the same atom are taken together to form an oxo.

In some embodiments of a compound of Formula (I), or a pharmaceutically acceptable salt, solvate, tautomer, or stereoisomer thereof, the compound is of Formula:

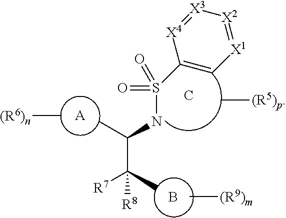

In some embodiments of a compound of Formula (I), Ring C is a 5- to 8-membered heterocycloalkyl comprising one or two additional heteroatoms selected from the group consisting of —O—, —S—, and —NR¹⁰—. In some embodiments of a compound of Formula (I), Ring C is a 5- to 8-membered heterocycloalkyl comprising one or two additional heteroatoms selected from the group consisting of —S(═O)— and —S(═O)₂—. In some embodiments of a compound of Formula (I), Ring C is a 5- to 8-membered heterocycloalkyl comprising one additional heteroatom selected from the group consisting of —O—, —S—, and —NR¹⁰—. In some embodiments of a compound of Formula (I), Ring C is a 5- to 8-membered heterocycloalkyl comprising one additional heteroatom selected from the group consisting of —S(═O)— and —S(═O)₂—. In some embodiments of a compound of Formula (I), Ring C is a 5- to 8-membered heterocycloalkyl comprising one or two additional heteroatoms selected from the group consisting of —O— and —NR¹⁰—. In some embodiments of a compound of Formula (I), Ring C is a 5- to 8-membered heterocycloalkyl comprising one additional heteroatom selected from the group consisting of —O— and —NR¹⁰—. In some embodiments of a compound of Formula (I), Ring C is a 5- to 8-membered heterocycloalkyl comprising one additional heteroatom that is —O—. In some embodiments of a compound of Formula (I), Ring C is a 5- to 8-membered heterocycloalkyl comprising one additional heteroatom that is —NR¹⁰—. In some embodiments of a compound of Formula (I), Ring C is a 5- to 8-membered heterocycloalkyl comprising one additional heteroatom that is —S—. In some embodiments of a compound of Formula (I), Ring C is a 5- to 8-membered heterocycloalkyl comprising one additional heteroatom that is —S(═O)₂—. In some embodiments of a compound of Formula (I), Ring C is a 5- to 8-membered heterocycloalkyl comprising one additional heteroatom that is —S(═O)—.

In some embodiments of a compound of Formula (I), Ring C is a 5- to 7-membered heterocycloalkyl comprising one additional heteroatom selected from the group consisting of —O—, —S—, and —NR¹⁰—. In some embodiments of a compound of Formula (I), Ring C is a 5- to 7-membered heterocycloalkyl comprising one additional heteroatom selected from the group consisting of —S(═O)— and —S(═O)₂—. In some embodiments of a compound of Formula (I), Ring C is a 5- to 7-membered heterocycloalkyl comprising one additional heteroatom selected from the group consisting of —O— and —NR¹⁰—. In some embodiments of a compound of Formula (I), Ring C is a 5- to 7-membered heterocycloalkyl comprising one additional heteroatom that is —O—. In some embodiments of a compound of Formula (I), Ring C is a 5- to 7-membered heterocycloalkyl comprising one additional heteroatom that is —NR¹⁰—. In some embodiments of a compound of Formula (I), Ring C is a 5- to 7-membered heterocycloalkyl comprising one additional heteroatom that is —S—. In some embodiments of a compound of Formula (I), Ring C is a 5- to 7-membered heterocycloalkyl comprising one additional heteroatom that is —S(═O)₂—. In some embodiments of a compound of Formula (I), Ring C is a 5- to 7-membered heterocycloalkyl comprising one additional heteroatom that is —S(═O)—.

In some embodiments of a compound of Formula (I), Ring C is a 6- to 7-membered heterocycloalkyl comprising one additional heteroatom selected from the group consisting of —O—, —S—, and —NR¹⁰—. In some embodiments of a compound of Formula (I), Ring C is a 6- to 7-membered heterocycloalkyl comprising one additional heteroatom selected from the group consisting of —S(═O)— and —S(═O)₂—. In some embodiments of a compound of Formula (I), Ring C is a 6- to 7-membered heterocycloalkyl comprising one additional heteroatom selected from the group consisting of —O— and —NR¹⁰—. In some embodiments of a compound of Formula (I), Ring C is a 6- to 7-membered heterocycloalkyl comprising one additional heteroatom that is —O—. In some embodiments of a compound of Formula (I), Ring C is a 6- to 7-membered heterocycloalkyl comprising one additional heteroatom that is —NR¹⁰—. In some embodiments of a compound of Formula (I), Ring C is a 6- to 7-membered heterocycloalkyl comprising one additional heteroatom that is —S—. In some embodiments of a compound of Formula (I), Ring C is a 6- to 7-membered heterocycloalkyl comprising one additional heteroatom that is —S(═O)₂—. In some embodiments of a compound of Formula (I), Ring C is a 6- to 7-membered heterocycloalkyl comprising one additional heteroatom that is —S(═O)—.

In some embodiments of a compound of Formula (I), Ring C is a 6-membered heterocycloalkyl comprising one additional heteroatom selected from the group consisting of —O—, —S—, and —NR¹⁰—. In some embodiments of a compound of Formula (I), Ring C is a 6-membered heterocycloalkyl comprising one additional heteroatom selected from the group consisting of —S(═O)— and —S(═O)₂—. In some embodiments of a compound of Formula (I), Ring C is a 6-membered heterocycloalkyl comprising one additional heteroatom selected from the group consisting of —O— and —NR¹⁰—. In some embodiments of a compound of Formula (I), Ring C is a 6-membered heterocycloalkyl comprising one additional heteroatom that is —NR¹⁰—. In some embodiments of a compound of Formula (I), Ring C is a 6-membered heterocycloalkyl comprising one additional heteroatom that is —O—. In some embodiments of a compound of Formula (I), Ring C is a 6-membered heterocycloalkyl comprising one additional heteroatom that is —S(═O)₂—. In some embodiments of a compound of Formula (I), Ring C is a 6-membered heterocycloalkyl comprising one additional heteroatom that is —S(═O)—. In some embodiments of a compound of Formula (I), Ring C is a 6-membered heterocycloalkyl comprising one additional heteroatom that is —S—.

In some embodiments of a compound of Formula (I), Ring C is a 7-membered heterocycloalkyl comprising one additional heteroatom selected from the group consisting of —O—, —S—, and —NR¹⁰—. In some embodiments of a compound of Formula (I), Ring C is a 7-membered heterocycloalkyl comprising one additional heteroatom selected from the group consisting of —S(═O)— and —S(═O)₂—. In some embodiments of a compound of Formula (I), Ring C is a 7-membered heterocycloalkyl comprising one additional heteroatom selected from the group consisting of —O— and —NR¹⁰—. In some embodiments of a compound of Formula (I), Ring C is a 7-membered heterocycloalkyl comprising one additional heteroatom that is —NR¹⁰—. In some embodiments of a compound of Formula (I), Ring C is a 7-membered heterocycloalkyl comprising one additional heteroatom that is —O—. In some embodiments of a compound of Formula (I), Ring C is a 7-membered heterocycloalkyl comprising one additional heteroatom that is —S—. In some embodiments of a compound of Formula (I), Ring C is a 7-membered heterocycloalkyl comprising one additional heteroatom that is —S(═O)₂—. In some embodiments of a compound of Formula (I), Ring C is a 7-membered heterocycloalkyl comprising one additional heteroatom that is —S(═O)—. In some embodiments of a compound of Formula (I), Ring C is a 7-membered heterocycloalkyl comprising one additional heteroatom that is —S—.

In some embodiments of a compound of Formula (I), each R⁵ is independently deuterium, halogen, —CN, —OH, —OR, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I), each R⁵ is independently deuterium, halogen, —CN, C₁-C₆alkyl, C₁-C₆haloalkyl, or C₁-C₆deuteroalkyl. In some embodiments of a compound of Formula (I), each R⁵ is independently C₁-C₆alkyl or C₁-C₆haloalkyl. In some embodiments of a compound of Formula (I), each R⁵ is independently C₁-C₆alkyl.

In some embodiments of a compound of Formula (I), two R⁵ on the same carbon are taken together to form an oxo.

In some embodiments of a compound of Formula (I), two R⁵ on the same carbon are taken together to form a cycloalkyl or heterocycloalkyl; each optionally substituted with one or more R.

In some embodiments of a compound of Formula (I), two R⁵ on adjacent atoms are taken together to form a cycloalkyl, heterocycloalkyl, aryl, or heteroaryl; each optionally substituted with one or more R.

In some embodiments of a compound of Formula (I), one R⁵ and R¹⁰ are taken together to form a heterocycloalkyl, or heteroaryl; each optionally substituted with one or more R.

In some embodiments of a compound of Formula (I), p is 0-3. In some embodiments of a compound of Formula (I), p is 0-2. In some embodiments of a compound of Formula (I), p is 0 or 1. In some embodiments of a compound of Formula (I), p is 1 or 2. In some embodiments of a compound of Formula (I), p is 1-3. In some embodiments of a compound of Formula (I), p is 1. In some embodiments of a compound of Formula (I), p is 2. In some embodiments of a compound of Formula (I), p is 3.

In some embodiments of a compound of Formula (I), the compound of Formula (I) is of Formula (Ia):

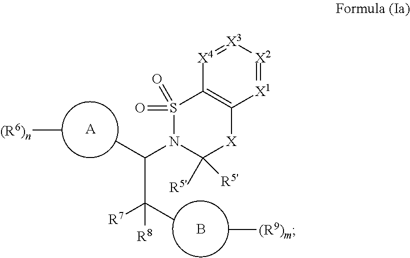

wherein:
X is —O—, —S—, —S(═O)—, —S(═O)₂—, or —NR¹⁰—.
each R^{5′} is independently hydrogen or R;
or two R^{5′} on the same carbon are taken together to form an oxo;
or two R^{5′} on the same carbon are taken together to form a cycloalkyl or heterocycloalkyl; each optionally substituted with one or more R;
or one R^{5′} and R¹⁰ are taken together to form a heterocycloalkyl or heteroaryl; each optionally substituted with one or more R.

In some embodiments of a compound of Formula (Ia), or a pharmaceutically acceptable salt, solvate, tautomer, or stereoisomer thereof, the compound is of Formula:

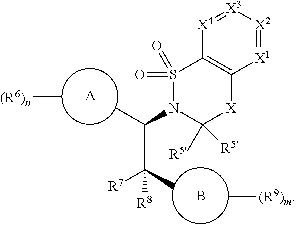

In some embodiments of a compound of Formula (I), the compound of Formula (I) is of Formula (Ib):

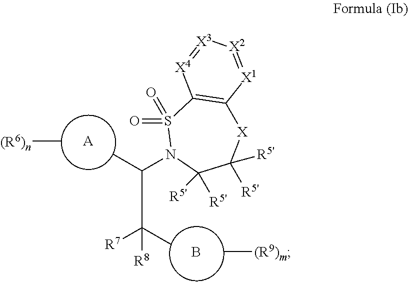

wherein:
X is —O—, —S—, —S(═O)—, —S(═O)₂—, or —NR¹⁰—.
each R^{5′} is independently hydrogen or R⁵;
or two R^{5′} on the same carbon are taken together to form an oxo;
or two R^{5′} on the same carbon are taken together to form a cycloalkyl or heterocycloalkyl; each optionally substituted with one or more R;
or two R^{5′} on adjacent carbons are taken together to form a cycloalkyl, heterocycloalkyl, aryl, or heteroaryl; each optionally substituted with one or more R;
or one R^{5′} and R¹⁰ are taken together to form a heterocycloalkyl or heteroaryl; each optionally substituted with one or more R.

In some embodiments of a compound of Formula (Ib), or a pharmaceutically acceptable salt, solvate, tautomer, or stereoisomer thereof, the compound is of Formula:

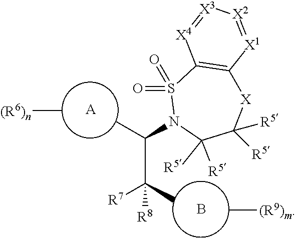

In some embodiments of a compound of Formula (I), the compound of Formula (I) is of Formula (Ic):

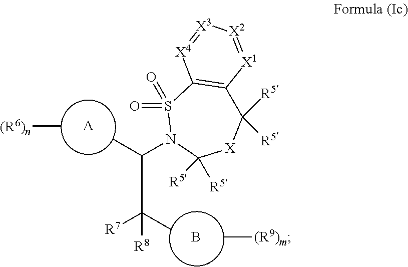

wherein:
X is —O—, —S—, —S(═O)—, —S(═O)₂—, or —NR¹⁰—.
each R^{5′} is independently hydrogen or R¹;
or two R^{5′} on the same carbon are taken together to form an oxo;
or two R^{5′} on the same carbon are taken together to form a cycloalkyl or heterocycloalkyl; each optionally substituted with one or more R;
or one R^{5′} and R¹⁰ are taken together to form a heterocycloalkyl or heteroaryl; each optionally substituted with one or more R.

In some embodiments of a compound of Formula (Ic), or a pharmaceutically acceptable salt, solvate, tautomer, or stereoisomer thereof, the compound is of Formula:

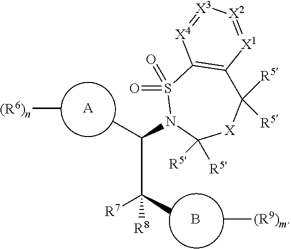

In some embodiments of a compound of Formula (Ia)-(Ic), X is —S—, —S(═O)—, or —S(═O)₂—. In some embodiments of a compound of Formula (Ia)-(Ic), X is —O—, —S—, or —NR¹⁰—. In some embodiments of a compound of Formula (Ia)-(Ic), X is —O— or —NR¹⁰—. In some embodiments of a compound of Formula (Ia)-(Ic), X is —O—. In some embodiments of a compound of Formula (Ia)-(Ic), X is —NR¹⁰.

In some embodiments of a compound of Formula (Ia)-(Ic), each R^{5′} is independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (Ia)-(Ic), each R^{5′} is independently hydrogen, deuterium, halogen, —CN, C₁-C₆alkyl, C₁-C₆haloalkyl, or C₁-C₆deuteroalkyl. In some embodiments of a compound of Formula (Ia)-(Ic), each R^{5′} is independently hydrogen, C₁-C₆alkyl, or C₁-C₆haloalkyl. In some embodiments of a compound of Formula (I), each R⁵ is independently hydrogen or C₁-C₆alkyl.

In some embodiments of a compound of Formula (Ia)-(Ic), two R^{5′} on the same carbon are taken together to form an oxo.

In some embodiments of a compound of Formula (Ia)-(Ic), two R^{5′} on the same carbon are taken together to form a cycloalkyl or heterocycloalkyl; each optionally substituted with one or more R.

In some embodiments of a compound of Formula (Ia)-(Ic), one R^{5′} and R¹⁰ are taken together to form a heterocycloalkyl or heteroaryl; each optionally substituted with one or more R.

In some embodiments of a compound of Formula (I) or (Ia)-(Ic), R¹⁰ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{10a}.

In some embodiments of a compound of Formula (I) or (Ia)-(Ic), R¹⁰ is hydrogen, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R^{10a}. In some embodiments of a compound of Formula (I) or (Ia)-(Ic), R¹⁰ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein the alkyl is optionally and independently substituted with one or more R^{1′}. In some embodiments of a compound of Formula (I) or (Ia)-(Ic), R¹⁰ is C₁-C₆alkyl. In some embodiments of a compound of Formula (I) or (Ia)-(Ic), each R^{10a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), each R^{10a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Ic), each R^{10a} is independently deuterium, halogen, —CN, —OH, —OR^{a}, —NR^{c}R^{d}, C₁-C₆alkyl, C₁-C₆haloalkyl, cycloalkyl, or heterocycloalkyl. In some embodiments of a compound of Formula (I) or (Ia)-(Ic), each R^{10a} is independently deuterium, halogen, —CN, —OH, —OR^{a}, —NR^{c}R^{d}, C₁-C₆alkyl, or C₁-C₆haloalkyl. In some embodiments of a compound of Formula (I) or (Ia)-(Ic), each R^{10a} is independently halogen, —OH, —NR^{c}R^{d}, C₁-C₆alkyl, C₁-C₆haloalkyl, or heterocycloalkyl.

In some embodiments of a compound of Formula (I) or (Ia)-(Ic), Ring A is a 5-membered heterocycloalkyl. In some embodiments of a compound of Formula (I) or (Ia)-(Ic), Ring A is a 5-membered heterocycloalkyl comprising one to four heteroatoms selected from the group consisting of O, S, and N. In some embodiments of a compound of Formula (I) or (Ia)-(Ic), Ring A is a 5-membered heterocycloalkyl comprising two to four heteroatoms selected from the group consisting of O, S, and N. In some embodiments of a compound of Formula (I) or (Ia)-(Ic), Ring A is a 5-membered heterocycloalkyl comprising three to four heteroatoms selected from the group consisting of O, S, and N. In some embodiments of a compound of Formula (I) or (Ia)-(Ic), Ring A is a 5-membered heteroaryl. In some embodiments of a compound of Formula (I) or (Ia)-(Ic), Ring A is a 5-membered heteroaryl comprising one to four heteroatoms selected from the group consisting of O, S, and N. In some embodiments of a compound of Formula (I) or (Ia)-(Ic), Ring A is a 5-membered heteroaryl comprising two to four heteroatoms selected from the group consisting of O, S, and N.

In some embodiments of a compound of Formula (I) or (Ia)-(Ic), Ring A is a 5-membered heteroaryl comprising three to four heteroatoms selected from the group consisting of O, S, and N. In some embodiments of a compound of Formula (I) or (Ia)-(Ic), Ring A is a triazole or tetrazole. In some embodiments of a compound of Formula (I) or (Ia)-(Ic), Ring A is a triazole. In some embodiments of a compound of Formula (I) or (Ia)-(Ic), Ring A is a tetrazole. In some embodiments of a compound of Formula (I) or (Ia)-(Ic), Ring A is a 2,3-dihydro-1,3,4-oxadiazole.

In some embodiments of a compound of Formula (I) or (Ia)-(Ic), each R⁶ is independently deuterium, halogen, —CN, —OH, —OR^{a}, —NR^{c}R^{d}, C₁-C₆alkyl, C₁-C₆haloalkyl, or C₁-C₆deuteroalkyl; or two R⁶ on the same atom are taken together to form an oxo. In some embodiments of a compound of Formula (I) or (Ia)-(Ic), each R⁶ is independently deuterium, halogen, or C₁-C₆alkyl; or two R⁶ on the same atom are taken together to form an oxo. In some embodiments of a compound of Formula (I) or (Ia)-(Ic), two R⁶ on the same atom are taken together to form an oxo.

In some embodiments of a compound of Formula (I) or (Ia)-(Ic), n is 0-3. In some embodiments of a compound of Formula (I) or (Ia)-(Ic), n is 0-2. In some embodiments of a compound of Formula (I) or (Ia)-(Ic), n is 0 or 1. In some embodiments of a compound of Formula (I) or (Ia)-(Ic), n is 2 or 3.

In some embodiments of a compound of Formula (I) or (Ia)-(Ic), n is 1-3. In some embodiments of a compound of Formula (I) or (Ia)-(Ic), n is 1. In some embodiments of a compound of Formula (I) or (Ia)-(Ic), n is 2. In some embodiments of a compound of Formula (I) or (Ia)-(Ic), n is 3.

In some embodiments of a compound of Formula (I) or (Ia)-(Ic),

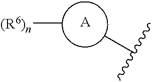

is

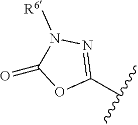

wherein R^{6′} is hydrogen or C₁-C₆alkyl. In some embodiments of a compound of Formula (I) or (Ia)-(Ic),

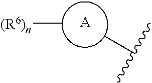

is

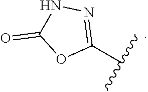 [SMILES: *c1n[nH]c(=O)o1]

In some embodiments of a compound of Formula (I) or (Ia)-(Ic), X¹ is CR¹. In some embodiments of a compound of Formula (I) or (Ia)-(Ic), X¹ is N.

In some embodiments of a compound of Formula (I) or (Ia)-(Ic), R¹ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R. In some embodiments of a compound of Formula (I) or (Ia)-(Ic), R¹ is hydrogen, deuterium, halogen, —CN, —OH, —OR^{a}, —NR^{c}R^{d}, —C(═O)R^{a}, C₁-C₆alkyl, C₁-C₆haloalkyl, C₁-C₆hydroxyalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R¹ is hydrogen, deuterium, halogen, —C(═O)R^{a}, C₁-C₆alkyl, C₁-C₆haloalkyl, or C₁-C₆hydroxyalkyl; wherein the alkyl is optionally and independently substituted with one or more R. In some embodiments of a compound of Formula (I) or (Ia)-(Ic), R¹ is hydrogen, deuterium, halogen, —CN, —OH, —OR^{a}, —NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Ic), R¹ is hydrogen, deuterium, halogen, —C(═O)R^{a}, C₁-C₆alkyl, C₁-C₆haloalkyl, or C₁-C₆hydroxyalkyl. In some embodiments of a compound of Formula (I) or (Ia)-(Ic), R¹ is hydrogen, —C(═O)R^{a}, C₁-C₆alkyl, or C₁-C₆hydroxyalkyl. In some embodiments of a compound of Formula (I) or (Ia)-(Ic), R¹ is hydrogen.

In some embodiments of a compound of Formula (I) or (Ia)-(Ic), X² is CR². In some embodiments of a compound of Formula (I) or (Ia)-(Ic), X² is N.

In some embodiments of a compound of Formula (I) or (Ia)-(Ic), R² is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R² is hydrogen, deuterium, halogen, —CN, —OH, —OR^{a}, —NR^{c}R^{d}, C₁-C₆alkyl, C₁-C₆haloalkyl, C₁-C₆deuteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R² is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I) or (Ia)-(Ic), R² is hydrogen, deuterium, halogen, —OH, C₁-C₆alkyl, or C₁-C₆haloalkyl. In some embodiments of a compound of Formula (I) or (Ia)-(Ic), R² is halogen. In some embodiments of a compound of Formula (I) or (Ia)-(Ic), R² is chloro.

In some embodiments of a compound of Formula (I) or (Ia)-(Ic), X³ is CR³. In some embodiments of a compound of Formula (I) or (Ia)-(Ic), X³ is N.

In some embodiments of a compound of Formula (I) or (Ia)-(Ic), R³ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R³ is hydrogen, deuterium, halogen, —CN, —OH, —OR^{a}, —NR^{c}R^{d}, —C₁-C₆alkyl, C₁-C₆haloalkyl, C₁-C₆hydroxyalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl.

In some embodiments of a compound of Formula (I) or (Ia)-(Ic), R³ is hydrogen, deuterium, halogen, C₁-C₆alkyl, C₁-C₆haloalkyl, or C₁-C₆hydroxyalkyl, C₁-C₆heteroalkyl. In some embodiments of a compound of Formula (I) or (Ia)-(Ic), R³ is hydrogen.

In some embodiments of a compound of Formula (I) or (Ia)-(Ic), X⁴ is CR⁴. In some embodiments of a compound of Formula (I) or (Ia)-(Ic), X⁴ is N.

In some embodiments of a compound of Formula (I) or (Ia)-(Ic), R⁴ is hydrogen, deuterium, halogen, —CN, OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R⁴ is hydrogen, deuterium, halogen, —CN, OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆heteroalkyl, cycloalkyl, or heterocycloalkyl; wherein the alkyl, cycloalkyl, and heterocycloalkyl is optionally and independently substituted with one or more R. In some embodiments of a compound of Formula (I) or (Ia)-(Ic), R⁴ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Ic), R⁴ is hydrogen, deuterium, halogen, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I) or (Ia)-(Ic), R⁴ is hydrogen, —C(═O)R^{a}, —C(═O)OR^{b}, —C(═O)NR^{c}R^{d}, C₁-C₆alkyl, C₁-C₆haloalkyl, C₁-C₆hydroxyalkyl, or C₁-C₆heteroalkyl. In some embodiments of a compound of Formula (I) or (Ia)-(Ic), R⁴ is hydrogen, —C(═O)R^{a}, —C(═O)OR^{b}, or —C(═O)NR^{c}R^{d}. In some embodiments of a compound of Formula (I) or (Ia)-(Ic), R⁴ is hydrogen or —C(═O)R^{a}. In some embodiments of a compound of Formula (I) or (Ia)-(Ic), R⁴ is hydrogen. In some embodiments of a compound of Formula (I) or (Ia)-(Ic), R⁴ is —C(═O)R^{a}.

In some embodiments of a compound of Formula (I) or (Ia)-(Ic), R⁷ is hydrogen, deuterium, halogen, C₁-C₆alkyl, C₁-C₆haloalkyl, or C₁-C₆deuteroalkyl. In some embodiments of a compound of Formula (I) or (Ia)-(Ic), R⁷ is C₁-C₆alkyl, C₁-C₆haloalkyl, or C₁-C₆deuteroalkyl. In some embodiments of a compound of Formula (I) or (Ia)-(Ic), R⁷ is C₁-C₆alkyl or C₁-C₆haloalkyl. In some embodiments of a compound of Formula (I) or (Ia)-(Ic), R⁷ is C₁-C₆alkyl. In some embodiments of a compound of Formula (I) or (Ia)-(Ic), R⁷ is halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I) or (Ia)-(Ic), R⁷ is C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl.

In some embodiments of a compound of Formula (I) or (Ia)-(Ic), R¹ is hydrogen or C₁-C₆alkyl. In some embodiments of a compound of Formula (I) or (Ia)-(Ic), R¹ is hydrogen.

In some embodiments of a compound of Formula (I) or (Ia)-(Ic), Ring B is aryl or heteroaryl. In some embodiments of a compound of Formula (I) or (Ia)-(Ic), Ring B is phenyl. In some embodiments of a compound of Formula (I) or (Ia)-(Ic), Ring B is 6-membered heteroaryl. In some embodiments of a compound of Formula (I) or (Ia)-(Ic), Ring B is pyridinyl.

In some embodiments of a compound of Formula (I) or (Ia)-(Ic), each R⁹ is independently deuterium, halogen, C₁-C₆alkyl, C₁-C₆haloalkyl, C₁-C₆deuteroalkyl, cycloalkyl, or heterocycloalkyl. In some embodiments of a compound of Formula (I) or (Ia)-(Ic), each R⁹ is independently halogen or C₁-C₆alkyl.

In some embodiments of a compound of Formula (I) or (Ia)-(Ic), m is 0-2. In some embodiments of a compound of Formula (I) or (Ia)-(Ic), m is 1 or 2. In some embodiments of a compound of Formula (I) or (Ia)-(Ic), m is 1-3. In some embodiments of a compound of Formula (I) or (Ia)-(Ic), m is 2 or 3. In some embodiments of a compound of Formula (I) or (Ia)-(Ic), m is 0. In some embodiments of a compound of Formula (I) or (Ia)-(Ic), m is 1. In some embodiments of a compound of Formula (I) or (Ia)-(Ic), m is 2. In some embodiments of a compound of Formula (I) or (Ia)-(Ic), m is 3.

In some embodiments of a compound of Formula (I) or (Ia)-(Ic),

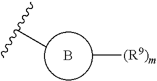

is

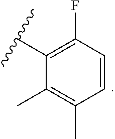 [SMILES: *c1c(F)ccc(C)c1C]

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, cycloalkyl, or heterocycloalkyl; wherein each alkyl, cycloalkyl, and heterocycloalkyl is independently optionally substituted with one or more R. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, cycloalkyl, or heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, cycloalkyl, or heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, cycloalkyl, or heterocycloalkyl; or two R on the same atom are taken together to form an oxo. In some embodiments of a compound disclosed herein, each R is independently halogen, —CN, —OH, —NH₂, —NHC₁-C₃alkyl, —N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, cycloalkyl, or heterocycloalkyl; or two R on the same atom are taken together to form an oxo. In some embodiments of a compound disclosed herein, each R is independently halogen, —CN, —OH, —NH₂, C₁-C₃alkyl, C₁-C₃alkoxy, C₁-C₃haloalkyl, C₁-C₃haloalkoxy, cycloalkyl, or heterocycloalkyl; or two R on the same atom are taken together to form an oxo. In some embodiments of a compound disclosed herein, each R is independently halogen, —CN, —OH, —NH₂, C₁-C₃alkyl, C₁-C₃alkoxy, C₁-C₃haloalkyl, or C₁-C₃haloalkoxy; or two R on the same atom are taken together to form an oxo. In some embodiments of a compound disclosed herein, each R is independently halogen, —CN, —OH, —NH₂, C₁-C₃alkyl, or C₁-C₃haloalkyl; or two R on the same atom are taken together to form an oxo. In some embodiments of a compound disclosed herein, each R is independently halogen, C₁-C₃alkyl, or C₁-C₃haloalkyl; or two R on the same atom are taken together to form an oxo. In some embodiments of a compound disclosed herein, each R is independently halogen or C₁-C₃alkyl; or two R on the same atom are taken together to form an oxo. In some embodiments of a compound disclosed herein, each R is independently halogen, —CN, —OH, —NH₂, C₁-C₃alkyl, C₁-C₃alkoxy, C₁-C₃haloalkyl, C₁-C₃haloalkoxy, cycloalkyl, or heterocycloalkyl. In some embodiments of a compound disclosed herein, each R is independently halogen, —CN, —OH, —NH₂, C₁-C₃alkyl, C₁-C₃alkoxy, C₁-C₃haloalkyl, or C₁-C₃haloalkoxy. In some embodiments of a compound disclosed herein, each R is independently halogen, —CN, —OH, —NH₂, C₁-C₃alkyl, or C₁-C₃haloalkyl. In some embodiments of a compound disclosed herein, each R is independently halogen, C₁-C₃alkyl, or C₁-C₃haloalkyl. In some embodiments of a compound disclosed herein, each R is independently halogen or C₁-C₃alkyl.

In some embodiments of a compound of Formula (I) or (Ia)-(Ic), the compound is selected from a compound of Table 1:

TABLE 1

| Example | Structure | Name |
|---|---|---|
| 1 | 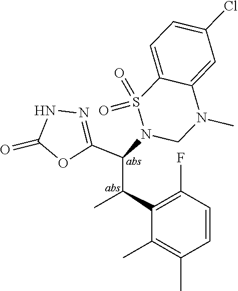 [SMILES: Cc1ccc(F)c([C@@H](C)[C@@H](c2n[nH]c(=O)o2)N2CN(C)c3cc(Cl)ccc3S2(=O)=O)c1C] | 5-((1S,2R)-1-(6-chloro-4-methyl-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 2 | 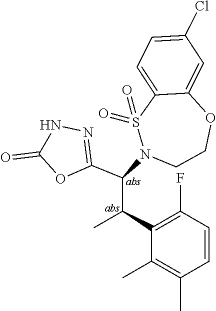 [SMILES: Cc1ccc(F)c([C@@H](C)[C@@H](c2n[nH]c(=O)o2)N2CCOc3cc(Cl)ccc3S2(=O)=O)c1C] | 5-((1S,2R)-1-(7-chloro-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 3 | 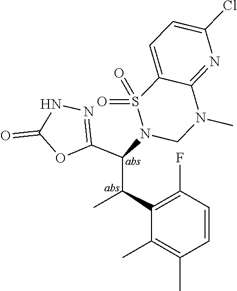 [SMILES: Cc1ccc(F)c([C@@H](C)[C@@H](c2n[nH]c(=O)o2)N2CN(C)c3nc(Cl)ccc3S2(=O)=O)c1C] | 5-((1S,2R)-1-(6-chloro-4-methyl-1,1- dioxido-3,4-dihydro-2H-pyrido[2,3- e][1,2,4]thiadiazin-2-y1)-2-(6-fluoro- 2,3-dimethylphenyl)propyl)-1,3,4- oxadiazol-2(3H)-one |
| 4 | 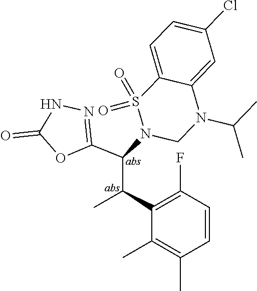 [SMILES: Cc1ccc(F)c([C@@H](C)[C@@H](c2n[nH]c(=O)o2)N2CN(C(C)C)c3cc(Cl)ccc3S2(=O)=O)c1C] | 5-((1S,2R)-1-(6-chloro-4-isopropyl-1,1- dioxido-3,4-dihydro-2H- benzo[e][1,2,4]thiadiazin-2-y1)-2-(6- fluoro-2,3-dimethylphenyl)propyl)- 1,3,4-oxadiazol-2(3H)-one |
| 5 | 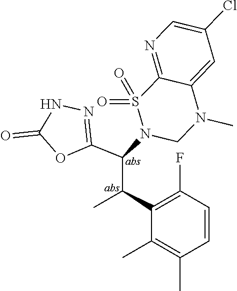 [SMILES: Cc1ccc(F)c([C@@H](C)[C@@H](c2n[nH]c(=O)o2)N2CN(C)c3cc(Cl)cnc3S2(=O)=O)c1C] | 5-((1S,2R)-1-(6-chloro-4-methyl-1,1- dioxido-3,4-dihydro-2H-pyrido[3,2- e][1,2,4]thiadiazin-2-yl)-2-(6-fluoro- 2,3-dimethylphenyl)propyl)-1,3,4- oxadiazol-2(3H)-one |
| 6 | 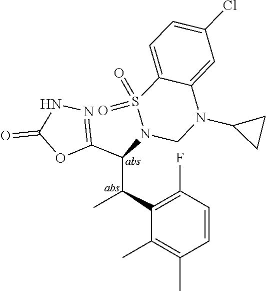 [SMILES: Cc1ccc(F)c([C@@H](C)[C@@H](c2n[nH]c(=O)o2)N2CN(C3CC3)c3cc(Cl)ccc3S2(=O)=O)c1C] | 5-((1S,2R)-1-(6-chloro-4-cyclopropyl- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 7 | 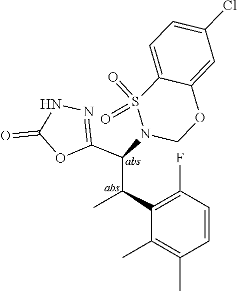 [SMILES: Cc1ccc(F)c([C@@H](C)[C@@H](c2n[nH]c(=O)o2)N2COc3cc(Cl)ccc3S2(=O)=O)c1C] | 5-((1S,2R)-1-(6-chloro-1,1- dioxidobenzo[e][1,4,3]oxathiazin- 2(3H)-y1)-2-(6-fluoro-2,3- dimethylphenyl)propyl)-1,3,4- oxadiazol-2(3H)-one |
| 8 | 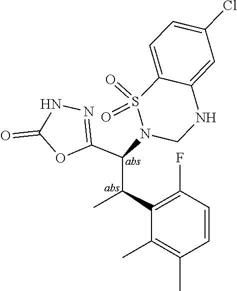 [SMILES: Cc1ccc(F)c([C@@H](C)[C@@H](c2n[nH]c(=O)o2)N2CNc3cc(Cl)ccc3S2(=O)=O)c1C] | 5-((1S,2R)-1-(6-chloro-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 9 | 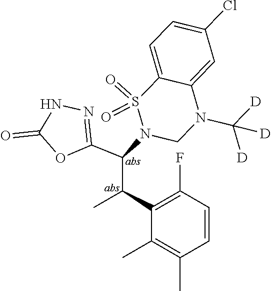 [SMILES: [2H]C([2H])([2H])N1CN([C@H](c2n[nH]c(=O)o2)[C@H](C)c2c(F)ccc(C)c2C)S(=O)(=O)c2ccc(Cl)cc21] | 5-((1S,2R)-1-(6-chloro-4-(methyl-d3)- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 10 | 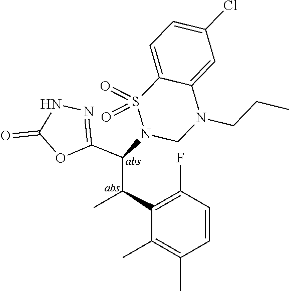 [SMILES: CCCN1CN([C@H](c2n[nH]c(=O)o2)[C@H](C)c2c(F)ccc(C)c2C)S(=O)(=O)c2ccc(Cl)cc21] | 5-((1S,2R)-1-(6-chloro-1,1-dioxido-4- propyl-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 11 | 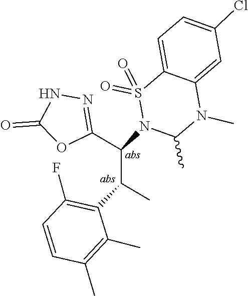 [SMILES: Cc1ccc(F)c([C@@H](C)[C@@H](c2n[nH]c(=O)o2)N2C(C)N(C)c3cc(Cl)ccc3S2(=O)=O)c1C] | 5-((1S,2R)-1-(6-chloro-3,4-dimethyl- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 12 | 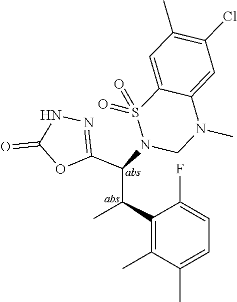 [SMILES: Cc1cc2c(cc1Cl)N(C)CN([C@H](c1n[nH]c(=O)o1)[C@H](C)c1c(F)ccc(C)c1C)S2(=O)=O] | 5-((1S,2R)-1-(6-chloro-4,7-dimethyl- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 13 | 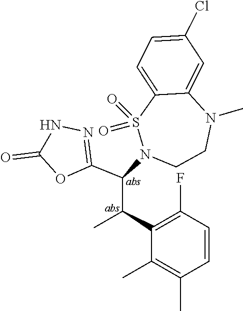 [SMILES: Cc1ccc(F)c([C@@H](C)[C@@H](c2n[nH]c(=O)o2)N2CCN(C)c3cc(Cl)ccc3S2(=O)=O)c1C] | 5-((1S,2R)-1-(7-chloro-5-methyl-1,1- dioxido-4,5- dihydrobenzo[f][1,2,5]thiadiazepin- 2(3H)-yl)-2-(6-fluoro-2,3- dimethylphenyl)propyl)-1,3,4- oxadiazol-2(3H)-one |
| 14 | 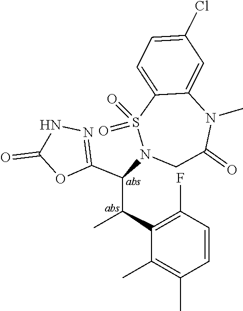 [SMILES: Cc1ccc(F)c([C@@H](C)[C@@H](c2n[nH]c(=O)o2)N2CC(=O)N(C)c3cc(Cl)ccc3S2(=O)=O)c1C] | 5-((1S,2R)-1-(7-chloro-5-methyl-1,1- dioxido-4-oxo-4,5- dihydrobenzo[f][1,2,5]thiadiazepin- 2(3H)-yl)-2-(6-fluoro-2,3- dimethylphenyl)propyl)-1,3,4- oxadiazol-2(3H)-one |
| 15 | 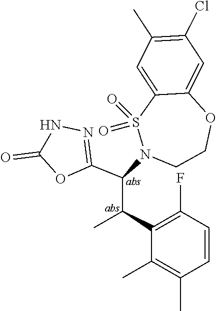 [SMILES: Cc1cc2c(cc1Cl)OCCN([C@H](c1n[nH]c(=O)o1)[C@H](C)c1c(F)ccc(C)c1C)S2(=O)=O] | 5-((1S,2R)-1-(7-chloro-8-methyl-1,1- dioxido-3,4-dihydro-2H- benzo[b][1,4,5]oxathiazepin-2-y1)-2-(6- fluoro-2,3-dimethylphenyl)propyl)- 1,3,4-oxadiazol-2(3H)-one |
| 16 | 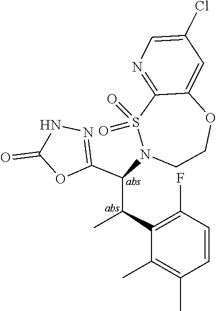 [SMILES: Cc1ccc(F)c([C@@H](C)[C@@H](c2n[nH]c(=O)o2)N2CCOc3cc(Cl)cnc3S2(=O)=O)c1C] | 5-((1S,2R)-1-(7-chloro-1,1-dioxido-3,4- dihydro-2H-pyrido[3,2- b][1,4,5]oxathiazepin-2-yl)-2-(6-fluoro- 2,3-dimethylphenyl)propyl)-1,3,4- oxadiazol-2(3H)-one |
| 17 | 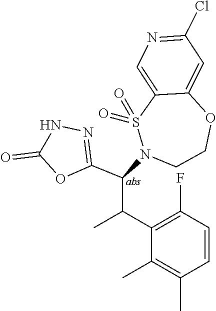 [SMILES: Cc1ccc(F)c(C(C)[C@@H](c2n[nH]c(=O)o2)N2CCOc3cc(Cl)ncc3S2(=O)=O)c1C] | 5-((1S, 2R)-1-(7-chloro-1,1-dioxido-3,4- dihydro-2H-pyrido[4,3- b][1,4,5]oxathiazepin-2-yl)-2-(6-fluoro- 2,3-dimethylphenyl)propyl)-1,3,4- oxadiazol-2(3H)-one |
| 18 | 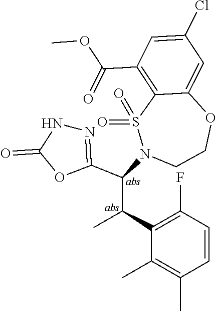 [SMILES: COC(=O)c1cc(Cl)cc2c1S(=O)(=O)N([C@H](c1n[nH]c(=O)o1)[C@H](C)c1c(F)ccc(C)c1C)CCO2] | methyl 7-chloro-2-((1S,2R)-2-(6-fluoro- 2,3-dimethylphenyl)-1-(5-oxo-4,5- dihydro-1,3,4-oxadiazol-2-yl)propyl)- 3,4-dihydro-2H- benzo[b][1,4,5]oxathiazepine-9- carboxylate 1,1-dioxide |
| 19 | 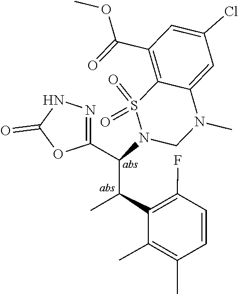 [SMILES: COC(=O)c1cc(Cl)cc2c1S(=O)(=O)N([C@H](c1n[nH]c(=O)o1)[C@H](C)c1c(F)ccc(C)c1C)CN2C] | methyl 6-chloro-2-((1S,2R)-2-(6-fluoro- 2,3-dimethylphenyl)-1-(5-oxo-4,5- dihydro-1,3,4-oxadiazol-2-yl)propyl)-4- methyl-3,4-dihydro-2H- benzo[e][1,2,4]thiadiazine-8- carboxylate 1,1-dioxide |
| 20 | 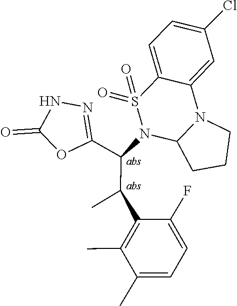 [SMILES: Cc1ccc(F)c([C@@H](C)[C@@H](c2n[nH]c(=O)o2)N2C3CCCN3c3cc(Cl)ccc3S2(=O)=O)c1C] | 5-((1S,2R)-1-(8-chloro-5,5-dioxido- 1,2,3,3a-tetrahydro-4H- benzo[e]pyrrolo[2,1-c][1,2,4]thiadiazin- 4-y1)-2-(6-fluoro-2,3- dimethylphenyl)propyl)-1,3,4- oxadiazol-2(3H)-one |
| 21 | 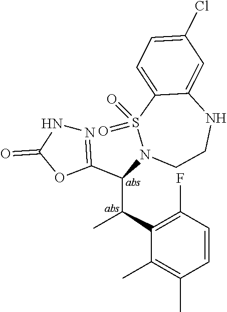 [SMILES: Cc1ccc(F)c([C@@H](C)[C@@H](c2n[nH]c(=O)o2)N2CCNc3cc(Cl)ccc3S2(=O)=O)c1C] | 5-((1S,2R)-1-(7-chloro-1,1-dioxido-4,5- dihydrobenzo[f][1,2,5]thiadiazepin- 2(3H)-y1)-2-(6-fluoro-2,3- dimethylphenyl)propyl)-1,3,4- oxadiazol-2(3H)-one |
| 22 | 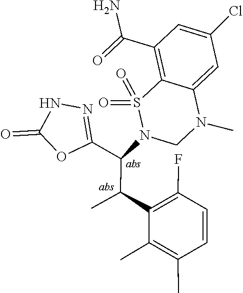 [SMILES: Cc1ccc(F)c([C@@H](C)[C@@H](c2n[nH]c(=O)o2)N2CN(C)c3cc(Cl)cc(C(N)=O)c3S2(=O)=O)c1C] | 6-chloro-2-((1S,2R)-2-(6-fluoro-2,3- dimethylpheny1)-1-(5-oxo-4,5-dihydro- 1,3,4-oxadiazol-2-yl)propyl)-4-methyl- 3,4-dihydro-2H- benzo[e][1,2,4]thiadiazine-8- carboxamide 1,1-dioxide |
| 23 | 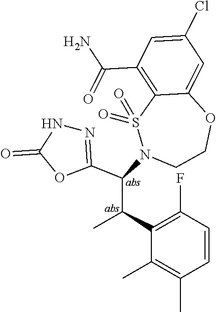 [SMILES: Cc1ccc(F)c([C@@H](C)[C@@H](c2n[nH]c(=O)o2)N2CCOc3cc(Cl)cc(C(N)=O)c3S2(=O)=O)c1C] | 7-chloro-2-((1S,2R)-2-(6-fluoro-2,3- dimethylphenyl)-1-(5-oxo-4,5-dihydro- 1,3,4-oxadiazol-2-yl)propyl)-3,4- dihydro-2H- benzo[b][1,4,5]oxathiazepine-9- carboxamide 1,1-dioxide |
| 24 | 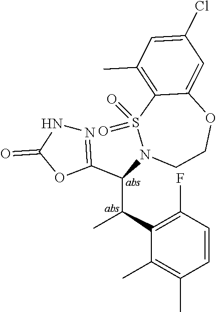 [SMILES: Cc1ccc(F)c([C@@H](C)[C@@H](c2n[nH]c(=O)o2)N2CCOc3cc(Cl)cc(C)c3S2(=O)=O)c1C] | 5-((1S,2R)-1-(7-chloro-9-methyl-1,1- dioxido-3,4-dihydro-2H- benzo[b][1,4,5]oxathiazepin-2-y1)-2-(6- fluoro-2,3-dimethylphenyl)propyl)- 1,3,4-oxadiazol-2(3H)-one |
| 25 | 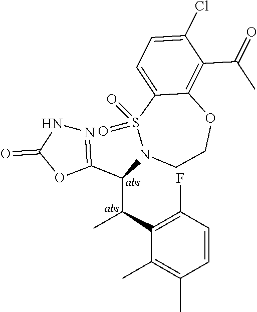 [SMILES: CC(=O)c1c(Cl)ccc2c1OCCN([C@H](c1n[nH]c(=O)o1)[C@H](C)c1c(F)ccc(C)c1C)S2(=O)=O] | 5-((1S,2R)-1-(6-acetyl-7-chloro-1,1- dioxido-3,4-dihydro-2H- benzo[b][1,4,5]oxathiazepin-2-y1)-2-(6- fluoro-2,3-dimethylphenyl)propyl)- 1,3,4-oxadiazol-2(3H)-one |
| 26 | 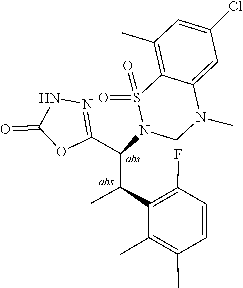 [SMILES: Cc1ccc(F)c([C@@H](C)[C@@H](c2n[nH]c(=O)o2)N2CN(C)c3cc(Cl)cc(C)c3S2(=O)=O)c1C] | 5-((1S,2R)-1-(6-chloro-4,8-dimethyl- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 27 | 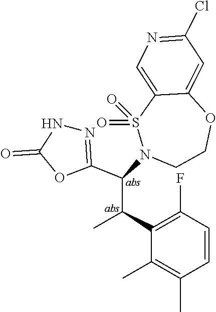 [SMILES: Cc1ccc(F)c([C@@H](C)[C@@H](c2n[nH]c(=O)o2)N2CCOc3cc(Cl)ncc3S2(=O)=O)c1C] | 5-((1S,2R)-1-(7-chloro-1,1-dioxido-3,4- dihydro-2H-pyrido[4,3- b][1,4,5]oxathiazepin-2-yl)-2-(6-fluoro- 2,3-dimethylphenyl)propyl)-1,3,4- oxadiazol-2(3H)-one |
| 28 | 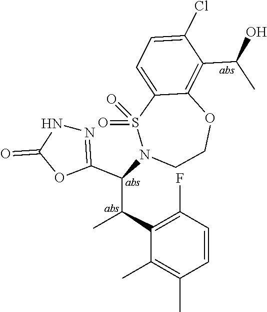 [SMILES: Cc1ccc(F)c([C@@H](C)[C@@H](c2n[nH]c(=O)o2)N2CCOc3c(ccc(Cl)c3[C@H](C)O)S2(=O)=O)c1C] | 5-((1S,2R)-1-(7-chloro-6-((S)-1- hydroxyethyl)-1,1-dioxido-3,4-dihydro- 2H-benzo[b][1,4,5]oxathiazepin-2-yl)-2- (6-fluoro-2,3-dimethylphenyl)propyl)- 1,3,4-oxadiazol-2(3H)-one |
| 29 | 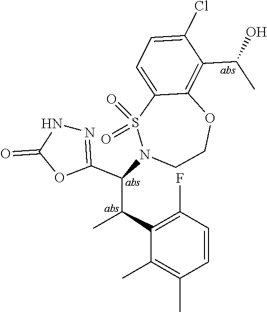 [SMILES: Cc1ccc(F)c([C@@H](C)[C@@H](c2n[nH]c(=O)o2)N2CCOc3c(ccc(Cl)c3[C@@H](C)O)S2(=O)=O)c1C] | 5-((1S,2R)-1-(7-chloro-6-((R)-1- hydroxyethyl)-1,1-dioxido-3,4-dihydro- 2H-benzo[b][1,4,5]oxathiazepin-2-y1)-2- (6-fluoro-2,3-dimethylphenyl)propyl)- 1,3,4-oxadiazol-2(3H)-one |
| 30 | 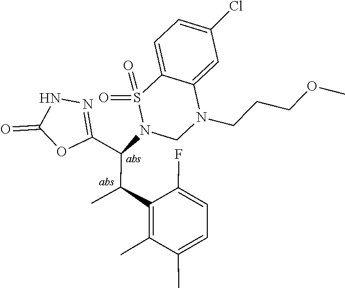 [SMILES: COCCCN1CN([C@H](c2n[nH]c(=O)o2)[C@H](C)c2c(F)ccc(C)c2C)S(=O)(=O)c2ccc(Cl)cc21] | 5-((1S,2R)-1-(6-chloro-4-(3- methoxypropyl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 31 | 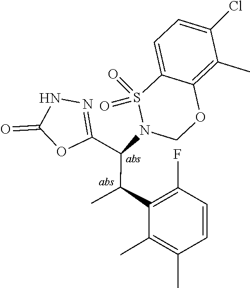 [SMILES: Cc1ccc(F)c([C@@H](C)[C@@H](c2n[nH]c(=O)o2)N2COc3c(ccc(Cl)c3C)S2(=O)=O)c1C] | 5-((1S,2R)-1-(6-chloro-5-methyl-1,1- dioxidobenzo[e][1,4,3]oxathiazin- 2(3H)-yl)-2-(6-fluoro-2,3- dimethylphenyl)propyl)-1,3,4- oxadiazol-2(3H)-one |
| 32 | 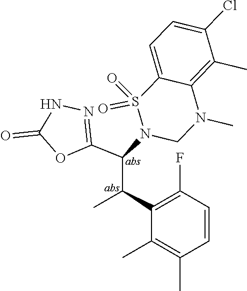 [SMILES: Cc1ccc(F)c([C@@H](C)[C@@H](c2n[nH]c(=O)o2)N2CN(C)c3c(ccc(Cl)c3C)S2(=O)=O)c1C] | 5-((1S,2R)-1-(6-chloro-4,5-dimethyl- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 33 | 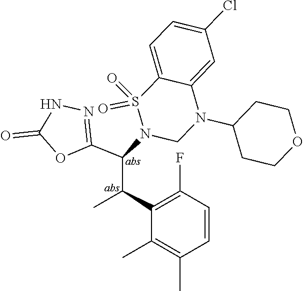 [SMILES: Cc1ccc(F)c([C@@H](C)[C@@H](c2n[nH]c(=O)o2)N2CN(C3CCOCC3)c3cc(Cl)ccc3S2(=O)=O)c1C] | 5-((1S,2R)-1-(6-chloro-1,1-dioxido-4- (tetrahydro-2H-pyran-4-yl)-3,4-dihydro- 2H-benzo[e][1,2,4]thiadiazin-2-yl)-2-(6- fluoro-2,3-dimethylphenyl)propyl)- 1,3,4-oxadiazol-2(3H)-one |
| 34 | 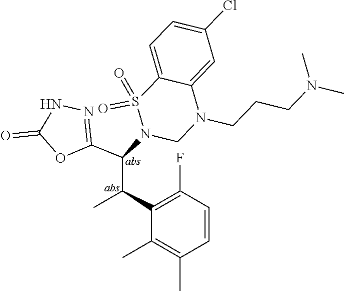 [SMILES: Cc1ccc(F)c([C@@H](C)[C@@H](c2n[nH]c(=O)o2)N2CN(CCCN(C)C)c3cc(Cl)ccc3S2(=O)=O)c1C] | 5-((1S,2R)-1-(6-chloro-4-(3- (dimethylamino)propyl)-1,1-dioxido- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 35 | 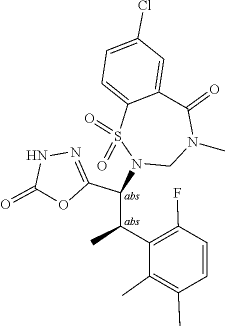 [SMILES: Cc1ccc(F)c([C@@H](C)[C@@H](c2n[nH]c(=O)o2)N2CN(C)C(=O)c3cc(Cl)ccc3S2(=O)=O)c1C] | 5-((1S,2R)-1-(7-chloro-4-methyl-1,1- dioxido-5-oxo-4,5- dihydrobenzo[f][1,2,4]thiadiazepin- 2(3H)-yl)-2-(6-fluoro-2,3- dimethylphenyl)propyl)-1,3,4- oxadiazol-2(3H)-one |
| 36 | 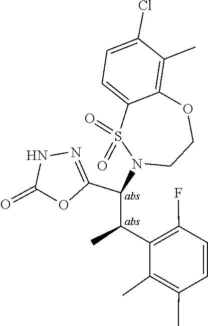 [SMILES: Cc1ccc(F)c([C@@H](C)[C@@H](c2n[nH]c(=O)o2)N2CCOc3c(ccc(Cl)c3C)S2(=O)=O)c1C] | 5-((1S,2R)-1-(7-chloro-6-methyl-1,1- dioxido-3,4-dihydro-2H- benzo[b][1,4,5]oxathiazepin-2-yl)-2-(6- fluoro-2,3-dimethylphenyl)propyl)- 1,3,4-oxadiazol-2(3H)-one |
| 37 | 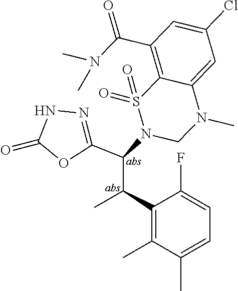 [SMILES: Cc1ccc(F)c([C@@H](C)[C@@H](c2n[nH]c(=O)o2)N2CN(C)c3cc(Cl)cc(C(=O)N(C)C)c3S2(=O)=O)c1C] | 6-chloro-2-((1S,2R)-2-(6-fluoro-2,3- dimethylphenyl)-1-(5-oxo-4,5-dihydro- 1,3,4-oxadiazol-2-yl)propyl)-N,N,4- trimethyl-3,4-dihydro-2H- benzo[e][1,2,4]thiadiazine-8- carboxamide 1,1-dioxide |
| 38 | 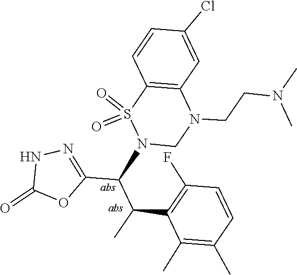 [SMILES: Cc1ccc(F)c([C@@H](C)[C@@H](c2n[nH]c(=O)o2)N2CN(CCN(C)C)c3cc(Cl)ccc3S2(=O)=O)c1C] | 5-((1S,2R)-1-(6-chloro-4-(2- (dimethylamino)ethyl)-1,1-dioxido-3,4- dihydro-2H-benzo[e][1,2,4]thiadiazin-2- y1)-2-(6-fluoro-2,3- dimethylphenyl)propyl)-1,3,4- oxadiazol-2(3H)-one |
| 39 | 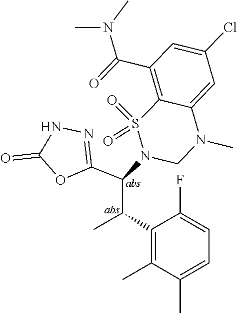 [SMILES: Cc1ccc(F)c([C@H](C)[C@@H](c2n[nH]c(=O)o2)N2CN(C)c3cc(Cl)cc(C(=O)N(C)C)c3S2(=O)=O)c1C] | 6-chloro-2-((1S,2S)-2-(6-fluoro-2,3- dimethylphenyl)-1-(5-oxo-4,5-dihydro- 1,3,4-oxadiazol-2-yl)propyl)-N,N,4- trimethyl-3,4-dihydro-2H- benzo[e][1,2,4]thiadiazine-8- carboxamide 1,1-dioxide |
| 40 | 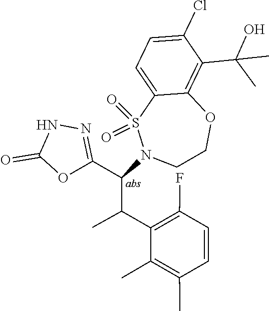 [SMILES: Cc1ccc(F)c(C(C)[C@@H](c2n[nH]c(=O)o2)N2CCOc3c(ccc(Cl)c3C(C)(C)O)S2(=O)=O)c1C] | 5-((1S)-1-(7-chloro-6-(2- hydroxypropan-2-y1)-1,1-dioxido-3,4- dihydro-2H- benzo[b][1,4,5]oxathiazepin-2-y1)-2-(6- fluoro-2,3-dimethylphenyl)propyl)- 1,3,4-oxadiazol-2(3H)-one |
| 41 | 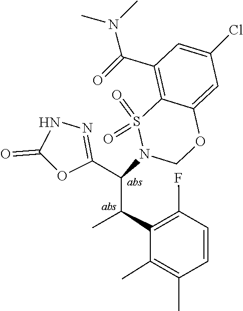 [SMILES: Cc1ccc(F)c([C@@H](C)[C@@H](c2n[nH]c(=O)o2)N2COc3cc(Cl)cc(C(=O)N(C)C)c3S2(=O)=O)c1C] | 6-chloro-2-((1S,2R)-2-(6-fluoro-2,3- dimethylphenyl)-1-(5-oxo-4,5-dihydro- 1,3,4-oxadiazol-2-yl)propyl)-N,N- dimethy1-2,3- dihydrobenzo[e][1,4,3]oxathiazine-8- carboxamide 1,1-dioxide |
| 42 | 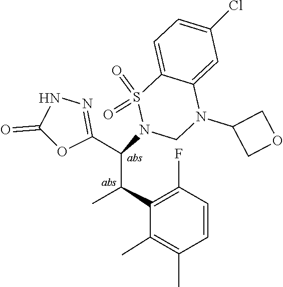 [SMILES: Cc1ccc(F)c([C@@H](C)[C@@H](c2n[nH]c(=O)o2)N2CN(C3COC3)c3cc(Cl)ccc3S2(=O)=O)c1C] | 5-((1S,2R)-1-(6-chloro-4-(oxetan-3-y1)- 1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 43 | 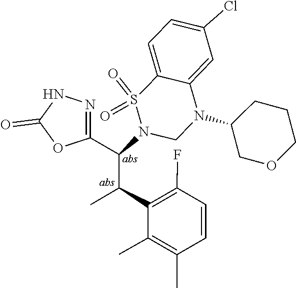 [SMILES: Cc1ccc(F)c([C@@H](C)[C@@H](c2n[nH]c(=O)o2)N2CN([C@@H]3CCCOC3)c3cc(Cl)ccc3S2(=O)=O)c1C] | 5-[(1S,2R)-1-[6-chloro-1,1-dioxo-4- [(3R)-tetrahydropyran-3-y1]-3H- 1λ6,2,4-benzothiadiazin-2-y1]-2-(6- fluoro-2,3-dimethyl-phenyl)propyl]-3H- 1,3,4-oxadiazol-2-one |
| 44 | 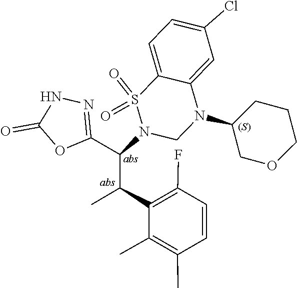 | 5-[(1S,2R)-1-[6-chloro-1,1-dioxo-4- [(3S)-tetrahydropyran-3-y1]-3H-126,2,4- benzothiadiazin-2-y1]-2-(6-fluoro-2,3- dimethyl-phenyl)propyl]-3H-1,3,4- oxadiazol-2-one |
| 45 | 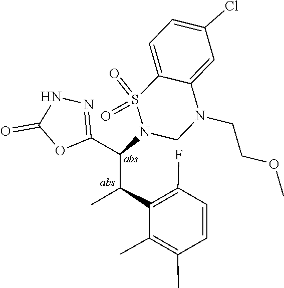 [SMILES: COCCN1CN([C@H](c2n[nH]c(=O)o2)[C@H](C)c2c(F)ccc(C)c2C)S(=O)(=O)c2ccc(Cl)cc21] | 5-[(1S,2R)-1-[6-chloro-4-(2- methoxyethyl)-1,1-dioxo-3H-126,2,4- benzothiadiazin-2-y1]-2-(6-fluoro-2,3- dimethyl-phenyl)propyl]-3H-1,3,4- oxadiazol-2-one |
| 46 | 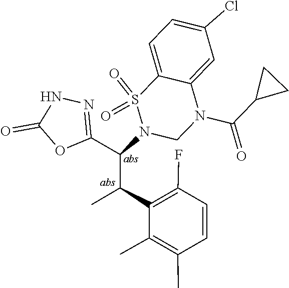 [SMILES: Cc1ccc(F)c([C@@H](C)[C@@H](c2n[nH]c(=O)o2)N2CN(C(=O)C3CC3)c3cc(Cl)ccc3S2(=O)=O)c1C] | 5-[(1S,2R)-1-[6-chloro-4- (cyclopropanecarbonyl)-1,1-dioxo-3H- 1λ6,2,4-benzothiadiazin-2-y1]-2-(6- fluoro-2,3-dimethyl-phenyl)propyl]-3H- 1,3,4-oxadiazol-2-one |
| 47 | 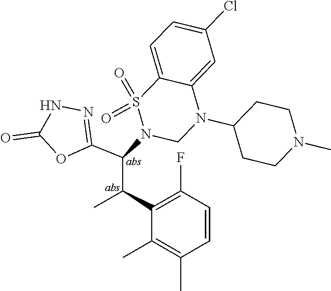 [SMILES: Cc1ccc(F)c([C@@H](C)[C@@H](c2n[nH]c(=O)o2)N2CN(C3CCN(C)CC3)c3cc(Cl)ccc3S2(=O)=O)c1C] | 5-[(1S,2R)-1-[6-chloro-4-(1-methyl-4- piperidyl)-1,1-dioxo-3H-1λ6,2,4- benzothiadiazin-2-yl]-2-(6-fluoro-2,3- dimethyl-phenyl)propyl]-3H-1,3,4- oxadiazol-2-one |
| 48 | 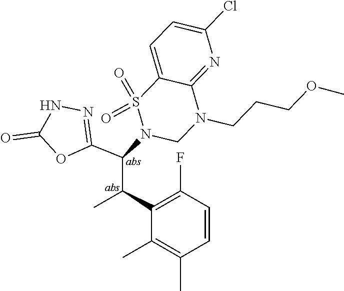 [SMILES: COCCCN1CN([C@H](c2n[nH]c(=O)o2)[C@H](C)c2c(F)ccc(C)c2C)S(=O)(=O)c2ccc(Cl)nc21] | 5-[(1S,2R)-1-[6-chloro-4-(3- methoxypropyl)-1,1-dioxo-3H- pyrido[2,3-e][1,2,4]thiadiazin-2-y1]-2- (6-fluoro-2,3-dimethyl-phenyl)propyl]- 3H-1,3,4-oxadiazol-2-one |
| 49 | 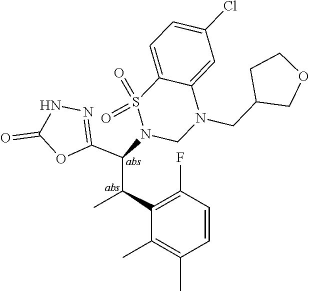 [SMILES: Cc1ccc(F)c([C@@H](C)[C@@H](c2n[nH]c(=O)o2)N2CN(CC3CCOC3)c3cc(Cl)ccc3S2(=O)=O)c1C] | 5-[(1S,2R)-1-[6-chloro-1,1-dioxo-4- (tetrahydrofuran-3-ylmethyl)-3H- 1λ6,2,4-benzothiadiazin-2-y1]-2-(6- fluoro-2,3-dimethyl-phenyl)propyl]-3H- 1,3,4-oxadiazol-2-one |
| 50 | 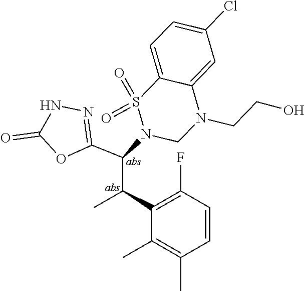 [SMILES: Cc1ccc(F)c([C@@H](C)[C@@H](c2n[nH]c(=O)o2)N2CN(CCO)c3cc(Cl)ccc3S2(=O)=O)c1C] | 5-[(1S,2R)-1-[6-chloro-4-(2- hydroxyethyl)-1,1-dioxo-3H-1λ6,2,4- benzothiadiazin-2-yl]-2-(6-fluoro-2,3- dimethyl-phenyl)propyl]-3H-1,3,4- oxadiazol-2-one |
| 51 | 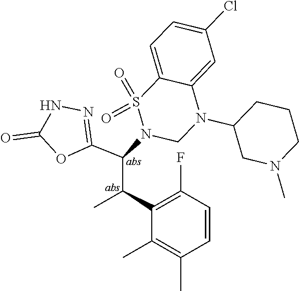 [SMILES: Cc1ccc(F)c([C@@H](C)[C@@H](c2n[nH]c(=O)o2)N2CN(C3CCCN(C)C3)c3cc(Cl)ccc3S2(=O)=O)c1C] | 5-[(1S,2R)-1-[6-chloro-4-(1-methyl-3- piperidyl)-1,1-dioxo-3H-116,2,4- benzothiadiazin-2-y1]-2-(6-fluoro-2,3- dimethyl-phenyl)propyl]-3H-1,3,4- oxadiazol-2-one |
| 52 | 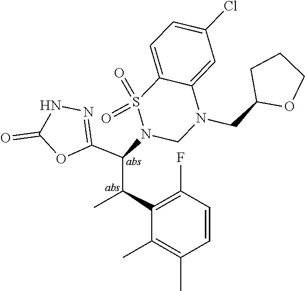 [SMILES: Cc1ccc(F)c([C@@H](C)[C@@H](c2n[nH]c(=O)o2)N2CN(C[C@H]3CCCO3)c3cc(Cl)ccc3S2(=O)=O)c1C] | 5-[(1S,2R)-1-[6-chloro-1,1-dioxo-4- [[(2R)-tetrahydrofuran-2-y1]methyl]-3H- 1λ6,2,4-benzothiadiazin-2-y1]-2-(6- fluoro-2,3-dimethyl-phenyl)propyl]-3H- 1,3,4-oxadiazol-2-one |
| 53 | 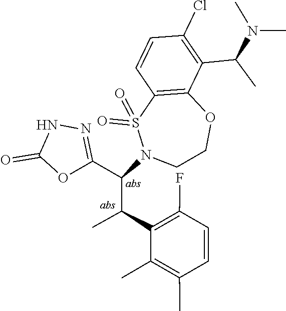 [SMILES: Cc1ccc(F)c([C@@H](C)[C@@H](c2n[nH]c(=O)o2)N2CCOc3c(ccc(Cl)c3[C@H](C)N(C)C)S2(=O)=O)c1C] | 5-((1S,2R)-1-(7-chloro-6-((S)-1- (dimethylamino)ethyl)-1,1-dioxido-3,4- dihydro-2H- benzo[b][1,4,5]oxathiazepin-2-y1)-2-(6- fluoro-2,3-dimethylphenyl)propyl)- 1,3,4-oxadiazol-2(3H)-one |
| 54 | 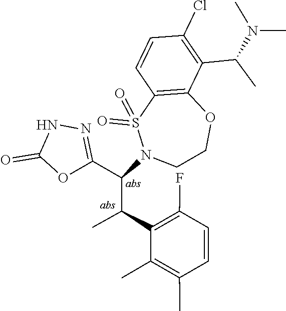 [SMILES: Cc1ccc(F)c([C@@H](C)[C@@H](c2n[nH]c(=O)o2)N2CCOc3c(ccc(Cl)c3[C@@H](C)N(C)C)S2(=O)=O)c1C] | 5-((1S,2R)-1-(7-chloro-6-((R)-1- (dimethylamino)ethyl)-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 55 | 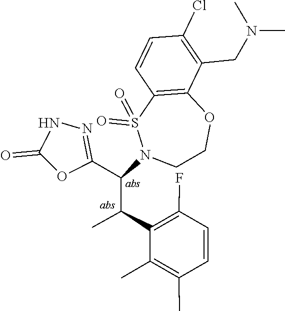 [SMILES: Cc1ccc(F)c([C@@H](C)[C@@H](c2n[nH]c(=O)o2)N2CCOc3c(ccc(Cl)c3CN(C)C)S2(=O)=O)c1C] | 5-((1S,2R)-1-(7-chloro-6- ((dimethylamino)methyl)-1,1-dioxido- 3,4-dihydro-2H- benzo[b][1,4,5]oxathiazepin-2-yl)-2-(6- fluoro-2,3-dimethylphenyl)propyl)- 1,3,4-oxadiazol-2(3H)-one |
| 56 | 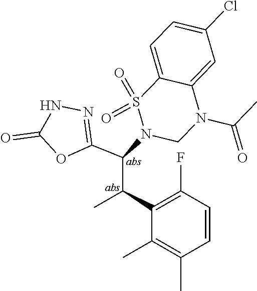 [SMILES: CC(=O)N1CN([C@H](c2n[nH]c(=O)o2)[C@H](C)c2c(F)ccc(C)c2C)S(=O)(=O)c2ccc(Cl)cc21] | 5-((1S,2R)-1-(4-acetyl-6-chloro-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 57 | 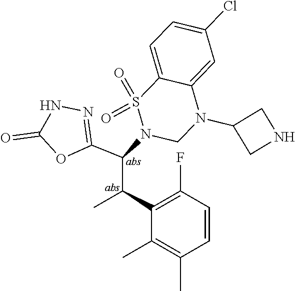 [SMILES: Cc1ccc(F)c([C@@H](C)[C@@H](c2n[nH]c(=O)o2)N2CN(C3CNC3)c3cc(Cl)ccc3S2(=O)=O)c1C] | 5-((1S,2R)-1-(4-(azetidin-3-y1)-6- chloro-1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 58 | 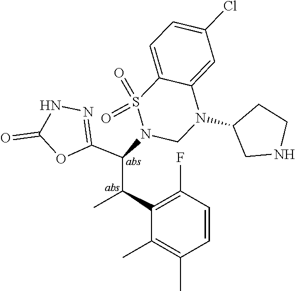 [SMILES: Cc1ccc(F)c([C@@H](C)[C@@H](c2n[nH]c(=O)o2)N2CN([C@@H]3CCNC3)c3cc(Cl)ccc3S2(=O)=O)c1C] | 5-((1S,2R)-1-(6-chloro-1,1-dioxido-4- ((R)-pyrrolidin-3-yl)-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 59 | 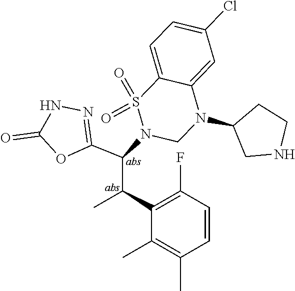 [SMILES: Cc1ccc(F)c([C@@H](C)[C@@H](c2n[nH]c(=O)o2)N2CN([C@H]3CCNC3)c3cc(Cl)ccc3S2(=O)=O)c1C] | 5-((1S,2R)-1-(6-chloro-1,1-dioxido-4- ((S)-pyrrolidin-3-y1)-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 60 | 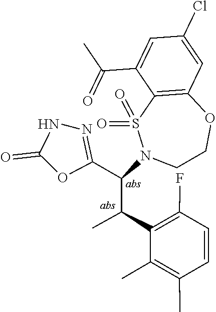 [SMILES: CC(=O)c1cc(Cl)cc2c1S(=O)(=O)N([C@H](c1n[nH]c(=O)o1)[C@H](C)c1c(F)ccc(C)c1C)CCO2] | 5-((1S,2R)-1-(9-acetyl-7-chloro-1,1- dioxido-3,4-dihydro-2H- benzo[b][1,4,5]oxathiazepin-2-yl)-2-(6- fluoro-2,3-dimethylphenyl)propyl)- 1,3,4-oxadiazol-2(3H)-one |
| 61 | 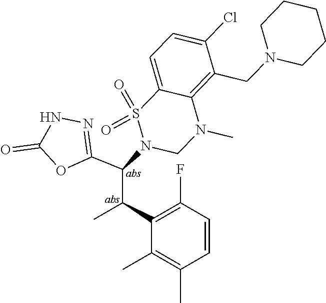 [SMILES: Cc1ccc(F)c([C@@H](C)[C@@H](c2n[nH]c(=O)o2)N2CN(C)c3c(ccc(Cl)c3CN3CCCCC3)S2(=O)=O)c1C] | 5-((1S,2R)-1-(6-chloro-4-methyl-1,1- dioxido-5-(piperidin-1-ylmethyl)-3,4- dihydro-2H-benzo[e][1,2,4]thiadiazin-2- y1)-2-(6-fluoro-2,3- dimethylphenyl)propyl)-1,3,4- oxadiazol-2(3H)-one |
| 62 | 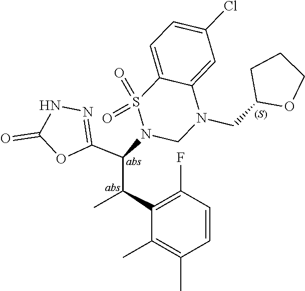 | 5-((1S,2R)-1-(6-chloro-1,1-dioxido-4- (((S)-tetrahydrofuran-2-yl)methyl)-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 63 | 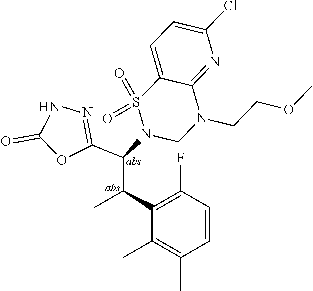 [SMILES: COCCN1CN([C@H](c2n[nH]c(=O)o2)[C@H](C)c2c(F)ccc(C)c2C)S(=O)(=O)c2ccc(Cl)nc21] | 5-((1S,2R)-1-(6-chloro-4-(2- methoxyethyl)-1,1-dioxido-3,4-dihydro- 2H-pyrido[2,3-e][1,2,4]thiadiazin-2-yl)- 2-(6-fluoro-2,3-dimethylphenyl)propyl)- 1,3,4-oxadiazol-2(3H)-one |
| 64 | 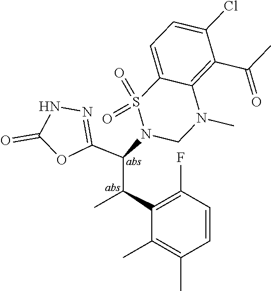 [SMILES: CC(=O)c1c(Cl)ccc2c1N(C)CN([C@H](c1n[nH]c(=O)o1)[C@H](C)c1c(F)ccc(C)c1C)S2(=O)=O] | 5-((1S,2R)-1-(5-acetyl-6-chloro-4- methyl-1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 65 | 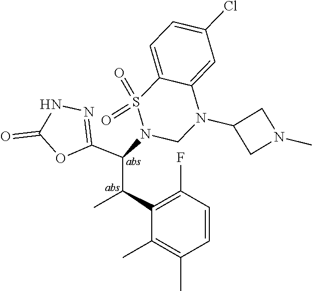 [SMILES: Cc1ccc(F)c([C@@H](C)[C@@H](c2n[nH]c(=O)o2)N2CN(C3CN(C)C3)c3cc(Cl)ccc3S2(=O)=O)c1C] | 5-((1S,2R)-1-(6-chloro-4-(1- methylazetidin-3-yl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 66 | 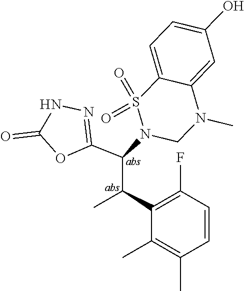 [SMILES: Cc1ccc(F)c([C@@H](C)[C@@H](c2n[nH]c(=O)o2)N2CN(C)c3cc(O)ccc3S2(=O)=O)c1C] | 5-((1S,2R)-2-(6-fluoro-2,3- dimethylpheny1)-1-(6-hydroxy-4- methyl-1,1-dioxido-3,4-dihydro-2H- benzo[e][1,2,4]thiadiazin-2-yl)propyl)- 1,3,4-oxadiazol-2(3H)-one |
| 67 | 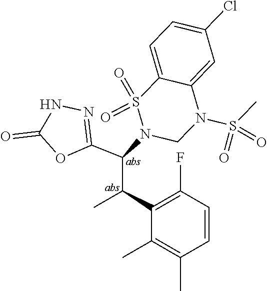 [SMILES: Cc1ccc(F)c([C@@H](C)[C@@H](c2n[nH]c(=O)o2)N2CN(S(C)(=O)=O)c3cc(Cl)ccc3S2(=O)=O)c1C] | 5-((1S,2R)-1-(6-chloro-4- (methylsulfony1)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 68 | 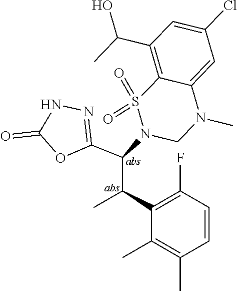 [SMILES: Cc1ccc(F)c([C@@H](C)[C@@H](c2n[nH]c(=O)o2)N2CN(C)c3cc(Cl)cc(C(C)O)c3S2(=O)=O)c1C] | 5-((1S,2R)-1-(6-chloro-8-(1- hydroxyethyl)-4-methyl-1,1-dioxido- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 69 | 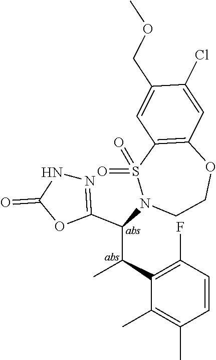 [SMILES: COCc1cc2c(cc1Cl)OCCN([C@H](c1n[nH]c(=O)o1)[C@H](C)c1c(F)ccc(C)c1C)S2(=O)=O] | 5-((1S,2R)-1-(7-chloro-8- (methoxymethyl)-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 70 | 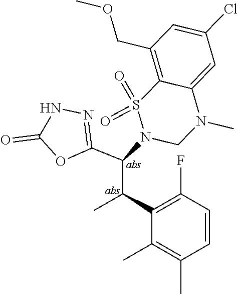 [SMILES: COCc1cc(Cl)cc2c1S(=O)(=O)N([C@H](c1n[nH]c(=O)o1)[C@H](C)c1c(F)ccc(C)c1C)CN2C] | 5-((1S,2R)-1-(6-chloro-8- (methoxymethyl)-4-methyl-1,1-dioxido- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 71 | 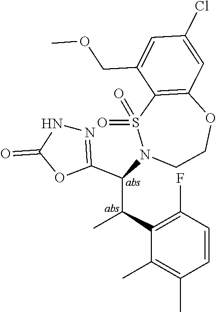 [SMILES: COCc1cc(Cl)cc2c1S(=O)(=O)N([C@H](c1n[nH]c(=O)o1)[C@H](C)c1c(F)ccc(C)c1C)CCO2] | 5-((1S,2R)-1-(7-chloro-9- (methoxymethyl)-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 72 | 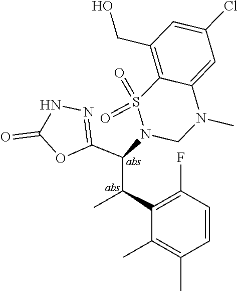 [SMILES: Cc1ccc(F)c([C@@H](C)[C@@H](c2n[nH]c(=O)o2)N2CN(C)c3cc(Cl)cc(CO)c3S2(=O)=O)c1C] | 5-((1S,2R)-1-(6-chloro-8- (hydroxymethyl)-4-methyl-1,1-dioxido- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 73 | 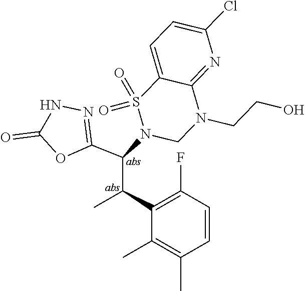 [SMILES: Cc1ccc(F)c([C@@H](C)[C@@H](c2n[nH]c(=O)o2)N2CN(CCO)c3nc(Cl)ccc3S2(=O)=O)c1C] | 5-((1S,2R)-1-(6-chloro-4-(2- hydroxyethyl)-1,1-dioxido-3,4-dihydro- 2H-pyrido[2,3-e][1,2,4]thiadiazin-2-yl)- 2-(6-fluoro-2,3-dimethylphenyl)propyl)- 1,3,4-oxadiazol-2(3H)-one |
| 74 | 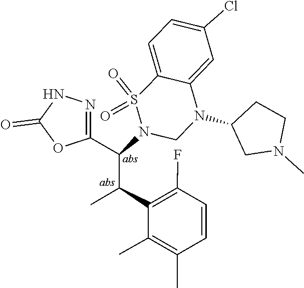 [SMILES: Cc1ccc(F)c([C@@H](C)[C@@H](c2n[nH]c(=O)o2)N2CN([C@@H]3CCN(C)C3)c3cc(Cl)ccc3S2(=O)=O)c1C] | 5-((1S,2R)-1-(6-chloro-4-((R)-1- methylpyrrolidin-3-yl)-1,1-dioxido-3,4- dihydro-2H-benzo[e][1,2,4]thiadiazin-2- y1)-2-(6-fluoro-2,3- dimethylphenyl)propyl)-1,3,4- oxadiazol-2(3H)-one |
| 75 | 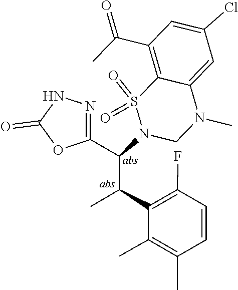 [SMILES: CC(=O)c1cc(Cl)cc2c1S(=O)(=O)N([C@H](c1n[nH]c(=O)o1)[C@H](C)c1c(F)ccc(C)c1C)CN2C] | 5-((1S,2R)-1-(8-acetyl-6-chloro-4- methyl-1,1-dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 76 | 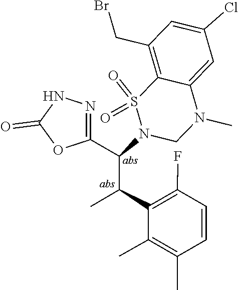 [SMILES: Cc1ccc(F)c([C@@H](C)[C@@H](c2n[nH]c(=O)o2)N2CN(C)c3cc(Cl)cc(CBr)c3S2(=O)=O)c1C] | 5-((1S,2R)-1-(8-(bromomethyl)-6- chloro-4-methyl-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 77 | 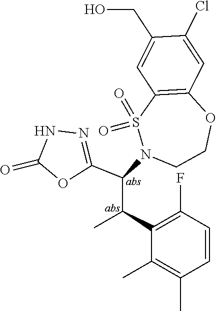 [SMILES: Cc1ccc(F)c([C@@H](C)[C@@H](c2n[nH]c(=O)o2)N2CCOc3cc(Cl)c(CO)cc3S2(=O)=O)c1C] | 5-((1S,2R)-1-(7-chloro-8- (hydroxymethyl)-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 78 | 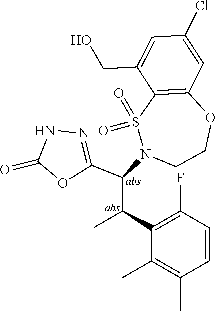 [SMILES: Cc1ccc(F)c([C@@H](C)[C@@H](c2n[nH]c(=O)o2)N2CCOc3cc(Cl)cc(CO)c3S2(=O)=O)c1C] | 5-((1S,2R)-1-(7-chloro-9- (hydroxymethyl)-1,1-dioxido-3,4- dihydro-2H- benzo[b][1,4,5]oxathiazepin-2-yl)-2-(6- fluoro-2,3-dimethylphenyl)propyl)- 1,3,4-oxadiazol-2(3H)-one |
| 79 | 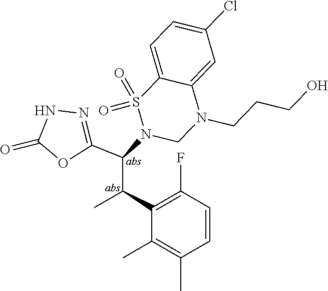 [SMILES: Cc1ccc(F)c([C@@H](C)[C@@H](c2n[nH]c(=O)o2)N2CN(CCCO)c3cc(Cl)ccc3S2(=O)=O)c1C] | 5-((1S,2R)-1-(6-chloro-4-(3- hydroxypropyl)-1,1-dioxido-3,4- dihydro-2H-benzo[e][1,2,4]thiadiazin-2- y1)-2-(6-fluoro-2,3- dimethylphenyl)propyl)-1,3,4- oxadiazol-2(3H)-one |
| 80 | 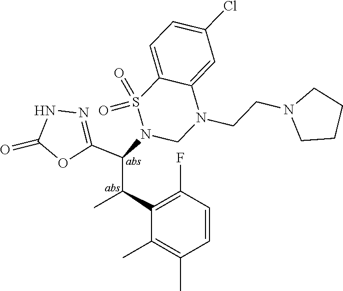 [SMILES: Cc1ccc(F)c([C@@H](C)[C@@H](c2n[nH]c(=O)o2)N2CN(CCN3CCCC3)c3cc(Cl)ccc3S2(=O)=O)c1C] | 5-((1S,2R)-1-(6-chloro-1,1-dioxido-4- (2-(pyrrolidin-1-yl)ethyl)-3,4-dihydro- 2H-benzo[e][1,2,4]thiadiazin-2-yl)-2-(6- fluoro-2,3-dimethylphenyl)propyl)- 1,3,4-oxadiazol-2(3H)-one |
| 81 | 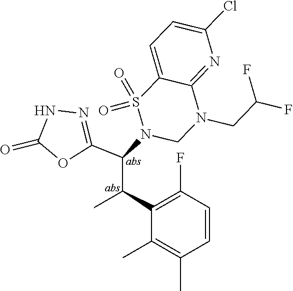 [SMILES: Cc1ccc(F)c([C@@H](C)[C@@H](c2n[nH]c(=O)o2)N2CN(CC(F)F)c3nc(Cl)ccc3S2(=O)=O)c1C] | 5-[(1S,2R)-1-[6-chloro-4-(2,2- difluoroethyl)-1,1-dioxo-3H-pyrido[2,3- e][1,2,4]thiadiazin-2-y1]-2-(6-fluoro- 2,3-dimethyl-phenyl)propyl]-3H-1,3,4- oxadiazol-2-one |
| 82 | 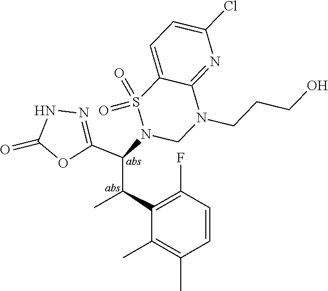 [SMILES: Cc1ccc(F)c([C@@H](C)[C@@H](c2n[nH]c(=O)o2)N2CN(CCCO)c3nc(Cl)ccc3S2(=O)=O)c1C] | 5-[(1S,2R)-1-[6-chloro-4-(3- hydroxypropyl)-1,1-dioxo-3H- pyrido[2,3-e][1,2,4]thiadiazin-2-y1]-2- (6-fluoro-2,3-dimethyl-phenyl)propyl]- 3H-1,3,4-oxadiazol-2-one |
| 83 | 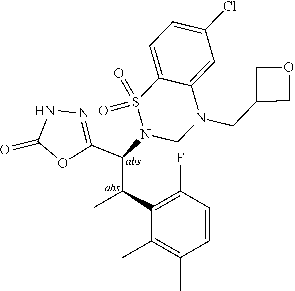 [SMILES: Cc1ccc(F)c([C@@H](C)[C@@H](c2n[nH]c(=O)o2)N2CN(CC3COC3)c3cc(Cl)ccc3S2(=O)=O)c1C] | 5-[(1S,2R)-1-[6-chloro-4-(oxetan-3- ylmethyl)-1,1-dioxo-3H-1λ6,2,4- benzothiadiazin-2-y1]-2-(6-fluoro-2,3- dimethyl-phenyl)propyl]-3H-1,3,4- oxadiazol-2-one |
| 84 | 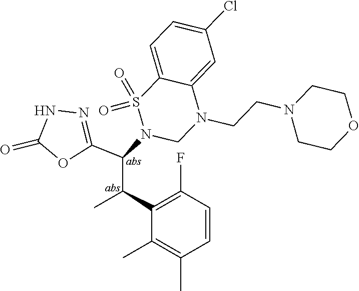 [SMILES: Cc1ccc(F)c([C@@H](C)[C@@H](c2n[nH]c(=O)o2)N2CN(CCN3CCOCC3)c3cc(Cl)ccc3S2(=O)=O)c1C] | 5-((1S,2R)-1-(6-chloro-4-(2- morpholinoethyl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 85 | 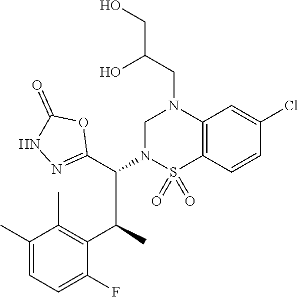 [SMILES: Cc1ccc(F)c([C@H](C)[C@H](c2n[nH]c(=O)o2)N2CN(CC(O)CO)c3cc(Cl)ccc3S2(=O)=O)c1C] | 5-((1R,2S)-1-(6-chloro-4-(2,3- dihydroxypropyl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 86 | 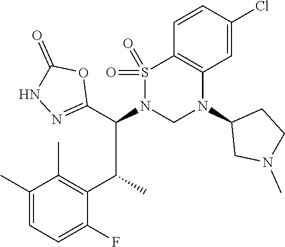 [SMILES: Cc1ccc(F)c([C@@H](C)[C@@H](c2n[nH]c(=O)o2)N2CN([C@H]3CCN(C)C3)c3cc(Cl)ccc3S2(=O)=O)c1C] | 5-[(1S,2R)-1-[6-chloro-4-[(3S)-1- methylpyrrolidin-3-yl]-1,1-dioxo-3H- 1λ6,2,4-benzothiadiazin-2-y1]-2-(6- fluoro-2,3-dimethyl-phenyl)propyl]-3H- 1,3,4-oxadiazol-2-one |
| 87 | 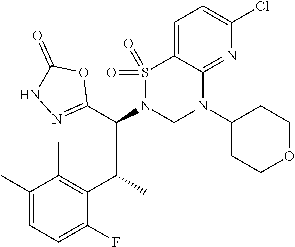 [SMILES: Cc1ccc(F)c([C@@H](C)[C@@H](c2n[nH]c(=O)o2)N2CN(C3CCOCC3)c3nc(Cl)ccc3S2(=O)=O)c1C] | 5-[(1S,2R)-1-(6-chloro-1,1-dioxo-4- tetrahydropyran-4-yl-3H-pyrido[2,3- e][1,2,4]thiadiazin-2-y1)-2-(6-fluoro- 2,3-dimethyl-phenyl)propyl]-3H-1,3,4- oxadiazol-2-one |
| 88 | 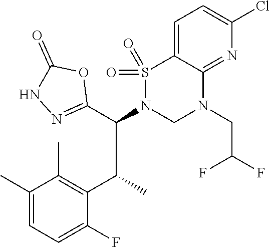 [SMILES: Cc1ccc(F)c([C@@H](C)[C@@H](c2n[nH]c(=O)o2)N2CN(CC(F)F)c3nc(Cl)ccc3S2(=O)=O)c1C] | 5-[(1S,2R)-1-[6-chloro-4-(2,2- difluoroethyl)-1,1-dioxo-3H-1λ6,2,4- benzothiadiazin-2-y1]-2-(6-fluoro-2,3- dimethyl-phenyl)propyl]-3H-1,3,4- oxadiazol-2-one |
| 89 | 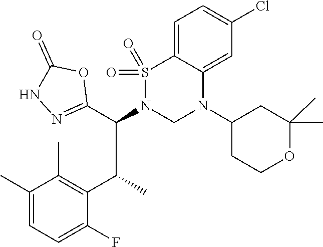 | 5-[(1S,2R)-1-[6-chloro-4-[(4R)-2,2- dimethyltetrahydropyran-4-y1]-1,1- dioxo-3H-1λ6,2,4-benzothiadiazin-2- yl]-2-(6-fluoro-2,3-dimethyl- phenyl)propyl]-3H-1,3,4-oxadiazol-2- one |
| 90 | 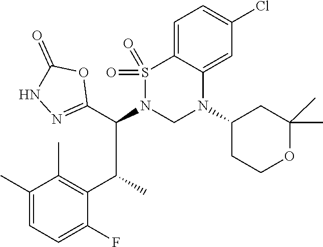 | 5-[(1S,2R)-1-[6-chloro-4-[(4S)-2,2- dimethyltetrahydropyran-4-y1]-1,1- dioxo-3H-1λ6,2,4-benzothiadiazin-2- yl]-2-(6-fluoro-2,3-dimethyl- phenyl)propyl]-3H-1,3,4-oxadiazol-2- one |
| 91 | 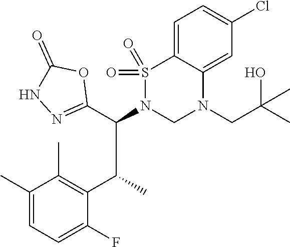 [SMILES: Cc1ccc(F)c([C@@H](C)[C@@H](c2n[nH]c(=O)o2)N2CN(CC(C)(C)O)c3cc(Cl)ccc3S2(=O)=O)c1C] | 5-[(1S,2R)-1-[6-chloro-4-(2-hydroxy-2- methyl-propyl)-1,1-dioxo-3H-1λ6,2,4- benzothiadiazin-2-yl]-2-(6-fluoro-2,3- dimethyl-phenyl)propyl]-3H-1,3,4- oxadiazol-2-one |
| 92 | 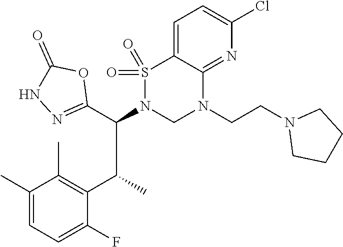 [SMILES: Cc1ccc(F)c([C@@H](C)[C@@H](c2n[nH]c(=O)o2)N2CN(CCN3CCCC3)c3nc(Cl)ccc3S2(=O)=O)c1C] | 5-[(1S,2R)-1-[6-chloro-1,1-dioxo-4-(2- pyrrolidin-1-ylethyl)-3H-pyrido[2,3- e][1,2,4]thiadiazin-2-y1]-2-(6-fluoro- 2,3-dimethyl-phenyl)propyl]-3H-1,3,4- oxadiazol-2-one |
| 93 | 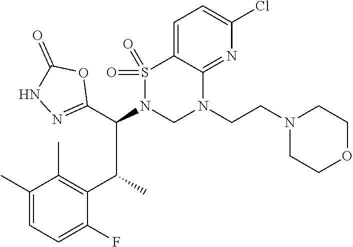 [SMILES: Cc1ccc(F)c([C@@H](C)[C@@H](c2n[nH]c(=O)o2)N2CN(CCN3CCOCC3)c3nc(Cl)ccc3S2(=O)=O)c1C] | 5-((1S,2R)-1-(6-chloro-4-(2- morpholinoethyl)-1,1-dioxido-3,4- dihydro-2H-pyrido[2,3- e][1,2,4]thiadiazin-2-yl)-2-(6-fluoro- 2,3-dimethylphenyl)propyl)-1,3,4- oxadiazol-2(3H)-one |
| 94 | 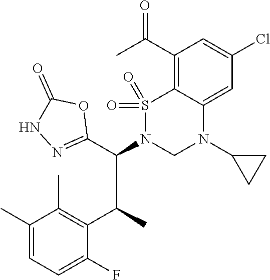 [SMILES: CC(=O)c1cc(Cl)cc2c1S(=O)(=O)N([C@H](c1n[nH]c(=O)o1)[C@@H](C)c1c(F)ccc(C)c1C)CN2C1CC1] | 5-((1S,2S)-1-(8-acetyl-6-chloro-4- cyclopropyl-1,1-dioxido-3,4-dihydro- 2H-benzo[e][1,2,4]thiadiazin-2-y1)-2-(6- fluoro-2,3-dimethylphenyl)propyl)- 1,3,4-oxadiazol-2(3H)-one |
| 95 | 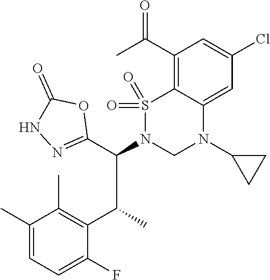 [SMILES: CC(=O)c1cc(Cl)cc2c1S(=O)(=O)N([C@H](c1n[nH]c(=O)o1)[C@H](C)c1c(F)ccc(C)c1C)CN2C1CC1] | 5-[(1S,2R)-1-(8-acetyl-6-chloro-4- cyclopropyl-1,1-dioxo-3H-1λ6,2,4- benzothiadiazin-2-yl)-2-(6-fluoro-2,3- dimethyl-phenyl)propyl]-3H-1,3,4- oxadiazol-2-one |
| 96 | 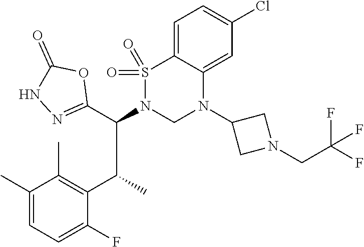 [SMILES: Cc1ccc(F)c([C@@H](C)[C@@H](c2n[nH]c(=O)o2)N2CN(C3CN(CC(F)(F)F)C3)c3cc(Cl)ccc3S2(=O)=O)c1C] | 5-((1S,2R)-1-(6-chloro-1,1-dioxido-4- (1-(2,2,2-trifluoroethyl)azetidin-3-yl)- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 97 | 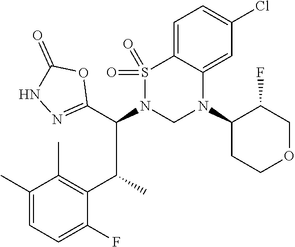 [SMILES: Cc1ccc(F)c([C@@H](C)[C@@H](c2n[nH]c(=O)o2)N2CN([C@@H]3CCOC[C@H]3F)c3cc(Cl)ccc3S2(=O)=O)c1C] | 5-((1S,2R)-1-(6-chloro-4-((3S,4R)-3- fluorotetrahydro-2H-pyran-4-yl)-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 98 | 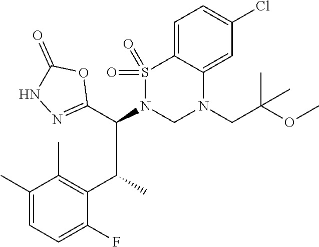 | 5-((1S,2R)-1-(6-chloro-4-(2-methoxy-2- methylpropyl)-1,1-dioxido-3,4-dihydro- 2H-benzo[e][1,2,4]thiadiazin-2-yl)-2-(6- fluoro-2,3-dimethylphenyl)propyl)- 1,3,4-oxadiazol-2(3H)-one |
| 99 | 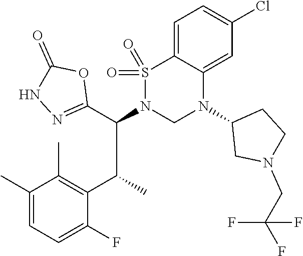 [SMILES: Cc1ccc(F)c([C@@H](C)[C@@H](c2n[nH]c(=O)o2)N2CN([C@@H]3CCN(CC(F)(F)F)C3)c3cc(Cl)ccc3S2(=O)=O)c1C] | 5-((1S,2R)-1-(6-chloro-1,1-dioxido-4- ((R)-1-(2,2,2-trifluoroethyl)pyrrolidin-3- yl)-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 100 | 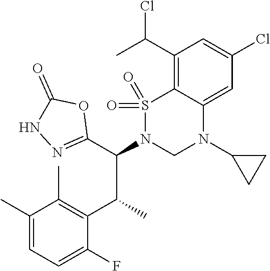 [SMILES: Cc1ccc(F)c([C@@H](C)[C@@H](c2n[nH]c(=O)o2)N2CN(C3CC3)c3cc(Cl)cc(C(C)Cl)c3S2(=O)=O)c1C] | 5-((1S,2R)-1-(6-chloro-8-(1- chloroethyl)-4-cyclopropyl-1,1-dioxido- 3,4-dihydro-2H- benzo[e][1,2,4]thiadiazin-2-y1)-2-(6- fluoro-2,3-dimethylphenyl)propyl)- 1,3,4-oxadiazol-2(3H)-one |
| 101 | 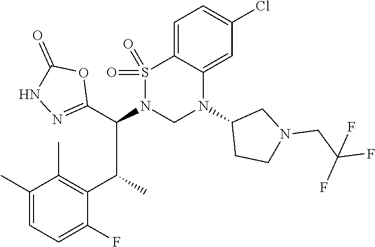 [SMILES: Cc1ccc(F)c([C@@H](C)[C@@H](c2n[nH]c(=O)o2)N2CN([C@H]3CCN(CC(F)(F)F)C3)c3cc(Cl)ccc3S2(=O)=O)c1C] | 5-((1S,2R)-1-(6-chloro-1,1-dioxido-4- ((S)-1-(2,2,2-trifluoroethyl)pyrrolidin-3- y1)-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 102 | 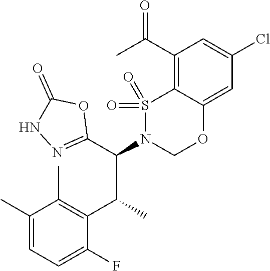 [SMILES: CC(=O)c1cc(Cl)cc2c1S(=O)(=O)N([C@H](c1n[nH]c(=O)o1)[C@H](C)c1c(F)ccc(C)c1C)CO2] | 5-((1S,2R)-1-(8-acetyl-6-chloro-1,1- dioxidobenzo[e][1,4,3]oxathiazin- 2(3H)-yl)-2-(6-fluoro-2,3- dimethylphenyl)propyl)-1,3,4- oxadiazol-2(3H)-one |
| 103 | 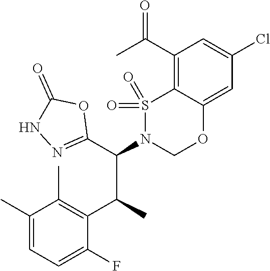 [SMILES: CC(=O)c1cc(Cl)cc2c1S(=O)(=O)N([C@H](c1n[nH]c(=O)o1)[C@@H](C)c1c(F)ccc(C)c1C)CO2] | 5-((1S,2S)-1-(8-acetyl-6-chloro-1,1- dioxidobenzo[e][1,4,3]oxathiazin- 2(3H)-yl)-2-(6-fluoro-2,3- dimethylphenyl)propyl)-1,3,4- oxadiazol-2(3H)-one |
| 104 | 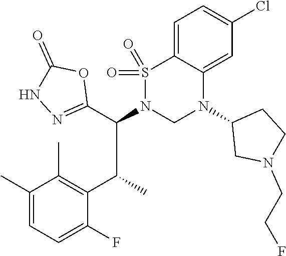 [SMILES: Cc1ccc(F)c([C@@H](C)[C@@H](c2n[nH]c(=O)o2)N2CN([C@@H]3CCN(CCF)C3)c3cc(Cl)ccc3S2(=O)=O)c1C] | 5-((1S,2R)-1-(6-chloro-4-((R)-1-(2- fluoroethyl)pyrrolidin-3-y1)-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 105 | 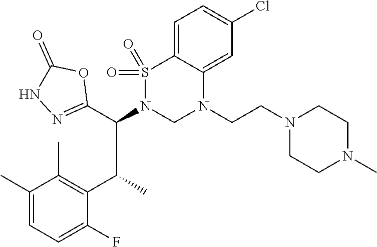 | 5-((1S,2R)-1-(6-chloro-4-(2-(4- methylpiperazin-1-yl)ethyl)-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 106 | 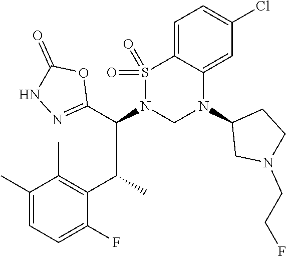 [SMILES: Cc1ccc(F)c([C@@H](C)[C@@H](c2n[nH]c(=O)o2)N2CN([C@H]3CCN(CCF)C3)c3cc(Cl)ccc3S2(=O)=O)c1C] | 5-((1S,2R)-1-(6-chloro-4-((S)-1-(2- fluoroethyl)pyrrolidin-3-y1)-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 107 | 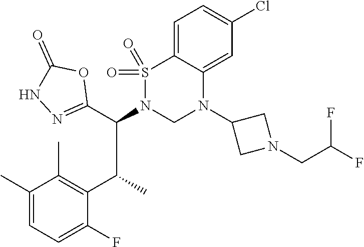 [SMILES: Cc1ccc(F)c([C@@H](C)[C@@H](c2n[nH]c(=O)o2)N2CN(C3CN(CC(F)F)C3)c3cc(Cl)ccc3S2(=O)=O)c1C] | 5-((1S,2R)-1-(6-chloro-4-(1-(2,2- difluoroethyl)azetidin-3-yl)-1,1-dioxido- 3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 108 | 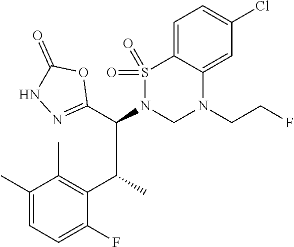 [SMILES: Cc1ccc(F)c([C@@H](C)[C@@H](c2n[nH]c(=O)o2)N2CN(CCF)c3cc(Cl)ccc3S2(=O)=O)c1C] | 5-((1S,2R)-1-(6-chloro-4-(2- fluoroethyl)-1,1-dioxido-3,4-dihydro- 2H-benzo[e][1,2,4]thiadiazin-2-y1)-2-(6- fluoro-2,3-dimethylphenyl)propyl)- 1,3,4-oxadiazol-2(3H)-one |
| 109 | 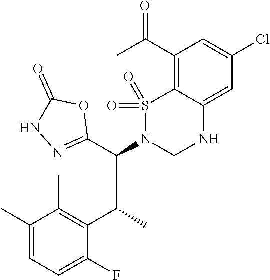 [SMILES: CC(=O)c1cc(Cl)cc2c1S(=O)(=O)N([C@H](c1n[nH]c(=O)o1)[C@H](C)c1c(F)ccc(C)c1C)CN2] | 5-((1S,2R)-1-(8-acetyl-6-chloro-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 110 | 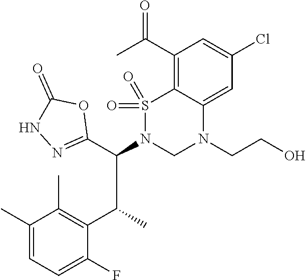 [SMILES: CC(=O)c1cc(Cl)cc2c1S(=O)(=O)N([C@H](c1n[nH]c(=O)o1)[C@H](C)c1c(F)ccc(C)c1C)CN2CCO] | 5-((1S,2R)-1-(8-acetyl-6-chloro-4-(2- hydroxyethyl)-1,1-dioxido-3,4-dihydro- 2H-benzo[e][1,2,4]thiadiazin-2-y1)-2-(6- fluoro-2,3-dimethylphenyl)propyl)- 1,3,4-oxadiazol-2(3H)-one |
| 111 | 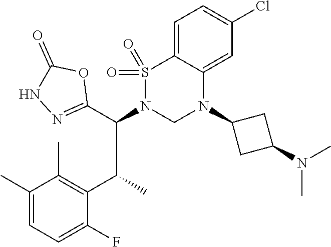 | 5-((1S,2R)-1-(6-chloro-4-((1s,3R)-3- (dimethylamino)cyclobutyl)-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 112 | 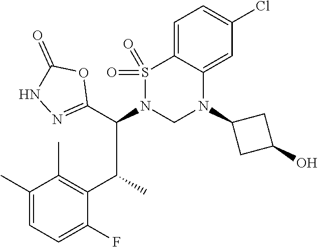 [SMILES: Cc1ccc(F)c([C@@H](C)[C@@H](c2n[nH]c(=O)o2)N2CN([C@H]3C[C@@H](O)C3)c3cc(Cl)ccc3S2(=O)=O)c1C] | 5-((1S,2R)-1-(6-chloro-4-((1s,3R)-3- hydroxycyclobutyl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 113 | 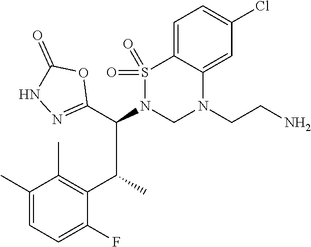 [SMILES: Cc1ccc(F)c([C@@H](C)[C@@H](c2n[nH]c(=O)o2)N2CN(CCN)c3cc(Cl)ccc3S2(=O)=O)c1C] | 5-((1S,2R)-1-(4-(2-aminoethyl)-6- chloro-1,1-dioxido-3,4-dihydro-2H- benzo[e][1,2,4]thiadiazin-2-y1)-2-(6- fluoro-2,3-dimethylphenyl)propyl)- 1,3,4-oxadiazol-2(3H)-one |
| 114 | 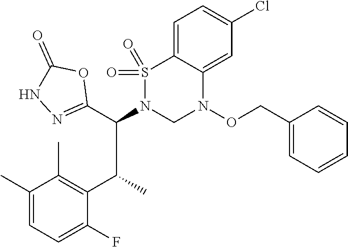 [SMILES: Cc1ccc(F)c([C@@H](C)[C@@H](c2n[nH]c(=O)o2)N2CN(OCc3ccccc3)c3cc(Cl)ccc3S2(=O)=O)c1C] | 5-((1S,2R)-1-(4-(benzyloxy)-6-chloro- 1,1-dioxido-3,4-dihydro-2H- benzo[e][1,2,4]thiadiazin-2-y1)-2-(6- fluoro-2,3-dimethylphenyl)propyl)- 1,3,4-oxadiazol-2(3H)-one |
| 115 | 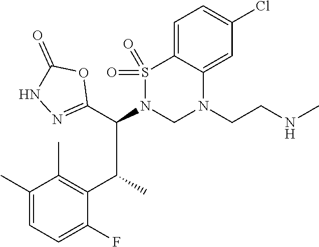 | 5-((1S,2R)-1-(6-chloro-4-(2- (methylamino)ethyl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |
| 116 | 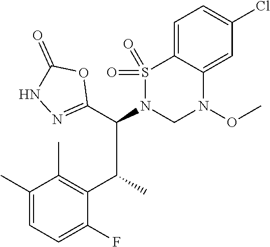 | 5-((1S,2R)-1-(6-chloro-4-methoxy-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 117 | 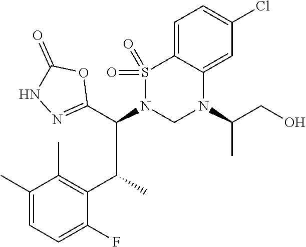 [SMILES: Cc1ccc(F)c([C@@H](C)[C@@H](c2n[nH]c(=O)o2)N2CN([C@H](C)CO)c3cc(Cl)ccc3S2(=O)=O)c1C] | 5-((1S,2R)-1-(6-chloro-4-((R)-1- hydroxypropan-2-yl)-1,1-dioxido-3,4- dihydro-2H-benzo[e][1,2,4]thiadiazin-2- y1)-2-(6-fluoro-2,3- dimethylphenyl)propyl)-1,3,4- oxadiazol-2(3H)-one |
| 118 | 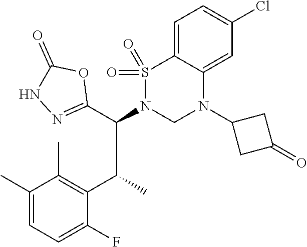 [SMILES: Cc1ccc(F)c([C@@H](C)[C@@H](c2n[nH]c(=O)o2)N2CN(C3CC(=O)C3)c3cc(Cl)ccc3S2(=O)=O)c1C] | 5-((1S,2R)-1-(6-chloro-1,1-dioxido-4- (3-oxocyclobutyl)-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 119 | 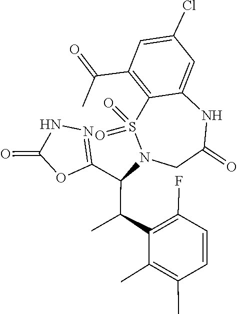 [SMILES: CC(=O)c1cc(Cl)cc2c1S(=O)(=O)N([C@H](c1n[nH]c(=O)o1)[C@H](C)c1c(F)ccc(C)c1C)CC(=O)N2] | 5-((1S,2R)-1-(9-acetyl-7-chloro-1,1- dioxido-4-oxo-4,5- dihydrobenzo[f][1,2,5]thiadiazepin- 2(3H)-y1)-2-(6-fluoro-2,3- dimethylphenyl)propyl)-1,3,4- oxadiazol-2(3H)-one |
| 120 | 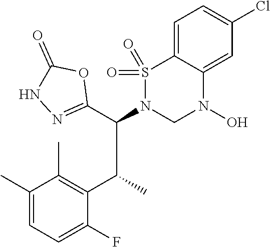 [SMILES: Cc1ccc(F)c([C@@H](C)[C@@H](c2n[nH]c(=O)o2)N2CN(O)c3cc(Cl)ccc3S2(=O)=O)c1C] | 5-((1S,2R)-1-(6-chloro-4-hydroxy-1,1- dioxido-3,4-dihydro-2H- benzo[e][1,2,4]thiadiazin-2-yl)-2-(6- fluoro-2,3-dimethylphenyl)propyl)- 1,3,4-oxadiazol-2(3H)-one |
| 121 | 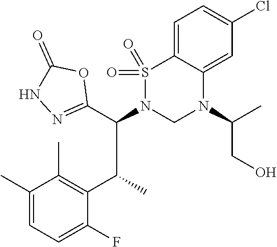 [SMILES: Cc1ccc(F)c([C@@H](C)[C@@H](c2n[nH]c(=O)o2)N2CN([C@@H](C)CO)c3cc(Cl)ccc3S2(=O)=O)c1C] | 5-((1S,2R)-1-(6-chloro-4-((S)-1- hydroxypropan-2-yl)-1,1-dioxido-3,4- dihydro-2H-benzo[e][1,2,4]thiadiazin-2- yl)-2-(6-fluoro-2,3- dimethylphenyl)propyl)-1,3,4- oxadiazol-2(3H)-one |

The absolute label (abs) is added to a chiral center to denote that it is unambiguously a pure sample of the drawn stereoisomer.

In some embodiments of a compound of Formula (I) or (Ia)-(Ic), the compound is selected from a compound of Table 2:

TABLE 2

| Structure | Structure |
|---|---|
| 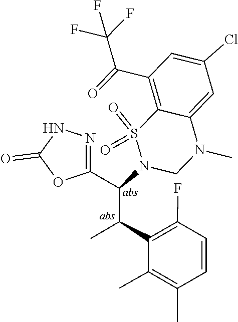 [SMILES: Cc1ccc(F)c([C@@H](C)[C@@H](c2n[nH]c(=O)o2)N2CN(C)c3cc(Cl)cc(C(=O)C(F)(F)F)c3S2(=O)=O)c1C] | 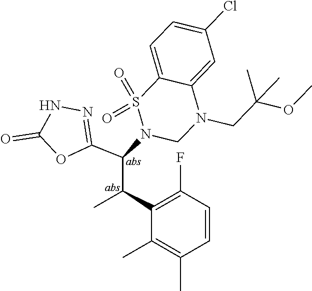 [SMILES: COC(C)(C)CN1CN([C@H](c2n[nH]c(=O)o2)[C@H](C)c2c(F)ccc(C)c2C)S(=O)(=O)c2ccc(Cl)cc21] |
| 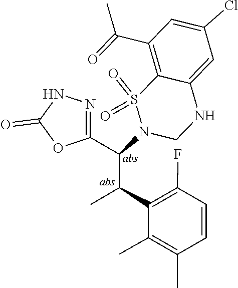 [SMILES: CC(=O)c1cc(Cl)cc2c1S(=O)(=O)N([C@H](c1n[nH]c(=O)o1)[C@H](C)c1c(F)ccc(C)c1C)CN2] | 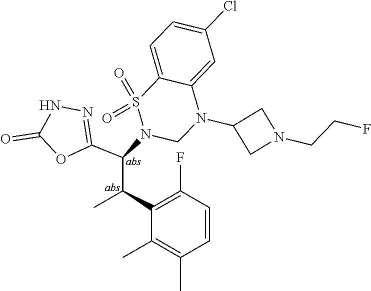 [SMILES: Cc1ccc(F)c([C@@H](C)[C@@H](c2n[nH]c(=O)o2)N2CN(C3CN(CCF)C3)c3cc(Cl)ccc3S2(=O)=O)c1C] |
| 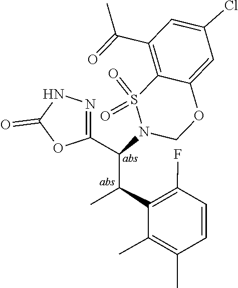 [SMILES: CC(=O)c1cc(Cl)cc2c1S(=O)(=O)N([C@H](c1n[nH]c(=O)o1)[C@H](C)c1c(F)ccc(C)c1C)CO2] | 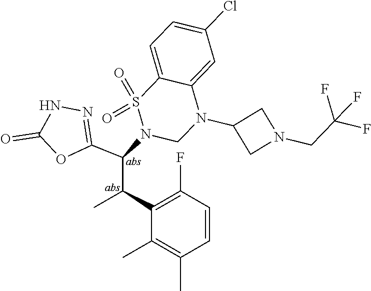 [SMILES: Cc1ccc(F)c([C@@H](C)[C@@H](c2n[nH]c(=O)o2)N2CN(C3CN(CC(F)(F)F)C3)c3cc(Cl)ccc3S2(=O)=O)c1C] |
| 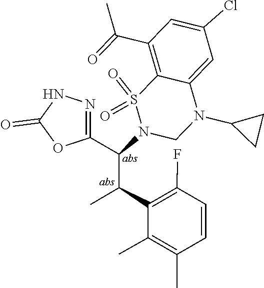 [SMILES: CC(=O)c1cc(Cl)cc2c1S(=O)(=O)N([C@H](c1n[nH]c(=O)o1)[C@H](C)c1c(F)ccc(C)c1C)CN2C1CC1] | 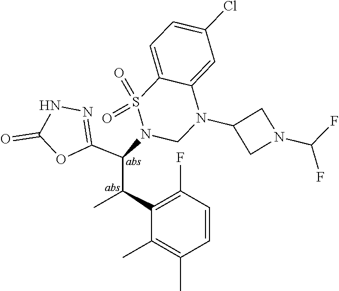 [SMILES: Cc1ccc(F)c([C@@H](C)[C@@H](c2n[nH]c(=O)o2)N2CN(C3CN(C(F)F)C3)c3cc(Cl)ccc3S2(=O)=O)c1C] |
| 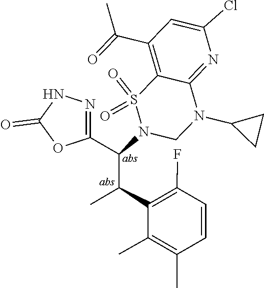 [SMILES: CC(=O)c1cc(Cl)nc2c1S(=O)(=O)N([C@H](c1n[nH]c(=O)o1)[C@H](C)c1c(F)ccc(C)c1C)CN2C1CC1] | 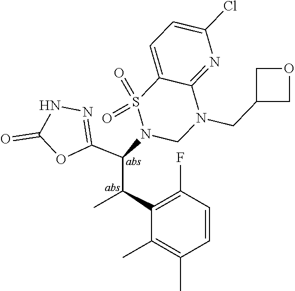 [SMILES: Cc1ccc(F)c([C@@H](C)[C@@H](c2n[nH]c(=O)o2)N2CN(CC3COC3)c3nc(Cl)ccc3S2(=O)=O)c1C] |
| 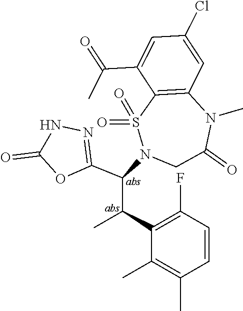 [SMILES: CC(=O)c1cc(Cl)cc2c1S(=O)(=O)N([C@H](c1n[nH]c(=O)o1)[C@H](C)c1c(F)ccc(C)c1C)CC(=O)N2C] | 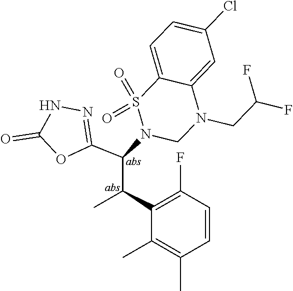 [SMILES: Cc1ccc(F)c([C@@H](C)[C@@H](c2n[nH]c(=O)o2)N2CN(CC(F)F)c3cc(Cl)ccc3S2(=O)=O)c1C] |
| 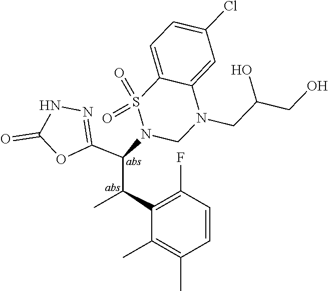 [SMILES: Cc1ccc(F)c([C@@H](C)[C@@H](c2n[nH]c(=O)o2)N2CN(CC(O)CO)c3cc(Cl)ccc3S2(=O)=O)c1C] | 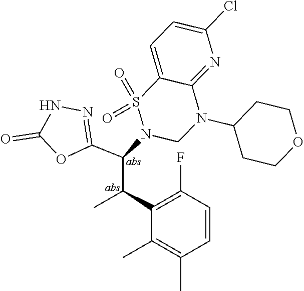 [SMILES: Cc1ccc(F)c([C@@H](C)[C@@H](c2n[nH]c(=O)o2)N2CN(C3CCOCC3)c3nc(Cl)ccc3S2(=O)=O)c1C] |
| 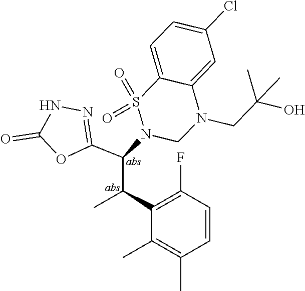 [SMILES: Cc1ccc(F)c([C@@H](C)[C@@H](c2n[nH]c(=O)o2)N2CN(CC(C)(C)O)c3cc(Cl)ccc3S2(=O)=O)c1C] | |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center independently exists in the R configuration or S configuration. The compounds described herein include all diastereomeric, enantiomeric, and epimeric forms as well as the corresponding mixtures thereof. The compounds described herein include all rotamers and “atropisomers as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions.

Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chlorine, such as ²H, ³C, ¹³C, ¹⁴C, ¹⁵N, ¹⁸Co, ¹⁷O, ³¹P, ³²p, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ⁴C are incorporated, are useful in drug and/or substrate tissue distribution assays. Tritium, i.e., ³H and carbon-14, i.e., ⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, one or more hydrogen in a compound disclosed herein has been replaced by a deuterium atom. In some embodiments, one or more alkyl substituents in a compound disclosed herein has been replaced by deuteroalkyl substituents.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, but not limited to, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, gluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁-C₄ alkyl)₄ hydroxide, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and one or more adjacent double bonds. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. In some embodiments, the tetrazoles disclosed herein exists as either of its tautomers:

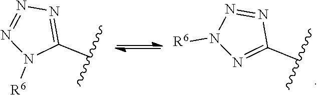

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Comwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Comwall, U.K.), Lancaster Synthesis (Windham, NH), Maybridge Chemical Co. Ltd. (Comwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts,” Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21° Ed. Mack Pub. Co., Easton, PA (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

Disclosed herein are methods for treating cancer in a subject in need thereof, including administering to the subject a therapeutically effective amount of a compound disclosed herein, or a pharmaceutically acceptable salt, solvate, tautomer, or stereoisomer thereof. Disclosed herein are methods for treating a RNR-related cancer in a subject in need thereof, including administering to the subject a therapeutically effective amount of a compound disclosed herein, or a pharmaceutically acceptable salt, solvate, tautomer, or stereoisomer thereof.

In some embodiments, the RNR-related cancer includes malignant tumors whose incidence can be decreased or whose symptom is in remission or alleviated and/or completely cured by deleting or suppressing and/or inhibiting functions of RNR. Malignant tumors of interest is, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), lung cancer (non-small cell lung cancer, small cell lung cancer, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors, skin cancer, brain tumor and the like.

In some embodiments, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, Herceptin (trastuzumab) resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus, Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, lobular carcinoma, ductal carcinoma, cancer of the pancreatic stellate cells, cancer of the hepatic stellate cells, or prostate cancer. In some embodiments, the cancer is selected from ovarian cancer, prostate cancer, esophageal cancer, salivary gland cancer, breast cancer, liver cancer, pancreatic cancer, stomach cancer, lung cancer, bladder cancer, colon cancer, and uterine cancer. In some embodiments, the cancer is selected from muscle cancer, brain cancer, lymph node cancer, thyroid cancer, kidney cancer, and adrenal gland cancer.

Also disclosed herein is a method of inhibiting ribonucleotide reductase in a subject, comprising administering to the subject a compound disclosed herein, or a pharmaceutically acceptable salt, solvate, tautomer, or stereoisomer thereof, or a pharmaceutical composition disclosed herein.

In some embodiments, the inhibition of ribonucleotide reductase occurs in a tumor cell in the subject in need thereof.

Also disclosed herein is a method for treating a tumor or tumor cells in a subject, the method comprising administering a compound disclosed herein, or a pharmaceutically acceptable salt, solvate, tautomer, or stereoisomer thereof, or the pharmaceutical composition disclosed herein, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, tautomer, or stereoisomer thereof, or the pharmaceutical composition disclosed herein, to a subject identified as having a tumor or tumor cells having ecDNA, wherein growth or size of the tumor or growth or number of the tumor cells is decreased as a result of treatment. In some embodiments, the method further comprises administering a cancer-targeted therapeutic agent. In some embodiments, the cancer-targeted therapeutic agent inhibits a gene or gene product comprised on ecDNA in the tumor or tumor cells.

Also disclosed herein is a method for treating a tumor or tumor cells in a subject, the method comprising administering a compound disclosed herein, or a pharmaceutically acceptable salt, solvate, tautomer, or stereoisomer thereof, or the pharmaceutical composition disclosed herein, in an amount sufficient to induce replication stress in the tumor or tumor cells, wherein the tumor or tumor cells comprises ecDNA or have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, tautomer, or stereoisomer thereof, or the pharmaceutical composition disclosed herein, to a subject identified as having a tumor or tumor cells having a focal amplification of an oncogene, wherein growth or size of the tumor or growth or number of the tumor cells is decreased as a result of treatment. In some embodiments, the method further comprises administering a cancer-targeted therapeutic agent, wherein the target of the therapeutic agent is a protein encoded by the oncogene. In some embodiments, the focal amplification is present on ecDNA.

ecDNA mediates an important and clinically distinct mechanism of resistance to targeted therapies. There are immediate therapeutic opportunities for utility of the one or more RNR inhibitor described herein as a single agent or in combination with other therapies. In some embodiments, the one or more RNR inhibitor described herein may be used to treat an ecDNA+ cancer, ecDNA+ tumor or ecDNA+ tumor cells. One or more RNR inhibitor described herein may be used to treat tumors, such as with one or more amplified oncogenes (e.g. FGFR, EGFR, MET, KRAS, MDM2 amplifications), in some cases, the one or more amplified oncogenes comprise non-mutant forms of the oncogene and in some cases, the amplified oncogenes comprises mutant forms of the oncogenes. In some cases, the tumor comprises one or more amplified oncogenes present on ecDNA and the one or more RNR inhibitor described herein are used to treat the tumor in combination with a therapeutic agent targeted to (e.g., an inhibitor of) the one or more amplified oncogenes on the ecDNA. One or more RNR inhibitor described herein may be used to treat tumors for which there are no approved targeted therapies or for which highly efficacious therapies are lacking. One or more RNR inhibitor described herein may be used to treat tumors that have developed resistance to another therapy such as a resistance to a targeted agent. In some cases, a tumor (or tumor cells) treated with one or more targeted agents develops resistance to a targeted agent, such as a targeted agent directed to an oncogene or a targeted agent that directly inhibits activating mutant forms of certain oncoproteins (e.g. KRAS, BRAF, EGFR) or as a consequence of focal amplification such as ecDNA-based amplification of the target gene itself, and the one or more RNR inhibitor described herein may be used to treat such tumors or tumor cells, alone or in combination with an additional therapeutic agent.

Provided herein are methods wherein inhibition of RNR by the one or more RNR inhibitors described herein exhibits synthetic lethality with a cancer-targeted agent. In some embodiments, synthetic lethality arises with one or more RNR inhibitors described herein in combination with a cancer targeted agent. In some cases, a tumor background is identified as hyper-sensitive to a RNR inhibitor and allows a sufficient therapeutic index to enable tolerated doses that are efficacious. In some embodiments, synthetic lethality arises with one or more RNR inhibitors described herein in combination with a cancer targeted agent where the tumor or tumor cells are ecDNA+. In some cases, RNR inhibition results in reduced ecDNA copy number. In some cases, RNR inhibition results in enhanced cytotoxicity in ecDNA+cells. In some cases, enhanced cytotoxicity results from the combination of RNR inhibition and inhibition of a cancer-target, such as an oncogene, for example an oncogene amplified on ecDNA. In an aspect of methods herein, a tumor or tumor cells to be treated are ecDNA+. In some cases, such tumor or tumor cells are determined to have an ecDNA signature. In some cases, a tumor or tumor cells are determined to have an ecDNA signature when the tumor or tumor cells have one or more characteristics associated with ecDNA+tumors or tumor cells. For example, in some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof.

In some embodiments, the compounds described herein, or a pharmaceutically acceptable salt, solvate, tautomer, or stereoisomer thereof, have good drug properties such as metabolic stability. In some embodiments, the compounds described herein, or a pharmaceutically acceptable salt, solvate, tautomer, or stereoisomer thereof, have long half-lives. In some embodiments, the compounds described herein, or a pharmaceutically acceptable salt, solvate, tautomer, or stereoisomer thereof, are stable in human hepatocytes. In some embodiments, the compounds described herein, or a pharmaceutically acceptable salt, solvate, tautomer, or stereoisomer thereof, have low clearance in human hepatocytes. In some embodiments, the compounds described herein, or a pharmaceutically acceptable salt, solvate, tautomer, or stereoisomer thereof, have a hepatocyte clearance (Clhep) value of less than about 10 mL/min/kg. In some embodiments, the compounds described herein, or a pharmaceutically acceptable salt, solvate, tautomer, or stereoisomer thereof, have a Clhep value that is less than about 20 mL/min/kg.

### Combination Therapy

In certain instances, the compound described herein, or a pharmaceutically acceptable salt, solvate, tautomer, or stereoisomer thereof, is administered in combination with a second therapeutic agent or a cancer-targeted agent.

In an aspect of methods herein, the method further comprises administering a cancer-targeted therapeutic agent, directed to an activity of a protein product of a target gene. In some cases, the treatment with the cancer-targeted therapeutic agent and the RNR inhibitor disclosed herein reduces amplification or expression of the target gene in the tumor or tumor cells. In some cases, the cancer-targeted therapeutic agent is administered prior to the RNR inhibitor. In some cases, the cancer-targeted therapeutic agent is administered concurrently with the RNR inhibitor.

In an aspect of methods herein, the tumor or tumor cells have an ecDNA signature. In some cases, the tumor or tumor cells develop the ecDNA signature after administration of the cancer-targeted therapeutic agent. In some cases, the tumor or tumor cells develop the ecDNA signature prior to treatment. In some cases, the method prevents an increase of ecDNA in the tumor or tumor cells.

In some embodiment, the second therapeutic includes antimetabolites, platinum drugs, plant alkaloid drugs, and molecular targeting drugs.

In some embodiments, the antimetabolites include 5-fluorouracil, 5-fluoro-2′-deoxyuridine, tegafur, tegafur-uracil, tegafur-gimeracil-oteracil, pemetrexed, trifluridine, trifluridine-tipiracil hydrochloride, fludarabine (or an active metabolite fludarabine nucleoside), cytarabine, gemcitabine, capecitabine, nelarabine, clofarabine, and DNA methylation inhibitors (decitabine, guadecitabine, azacitidine, etc.).

In some embodiments, the platinum drugs include cisplatin, oxaliplatin, carboplatin, and nedaplatin.

In some embodiments, the plant alkaloid drugs include microtube inhibiting drugs such as paclitaxel, docetaxel, vinblastine, vincristine, vindesine, vinorelbine, and eribulin, and topoisomerase inhibiting drugs such as irinotecan (or an active metabolite SN-38), nogitecan, and etoposide.

In some embodiments, the molecular targeting drugs include ATR (ataxia telangiectasia and Rad3 related protein) inhibitors, Chk1 (checkpoint kinase 1) inhibitors, HSP (heat shock protein) 90 inhibitors, PARP (poly ADP ribose polymerase) inhibitors, EGFR (epidermal growth factor receptor) inhibitors, Her2 inhibitors, VEGFR (vascular endothelial growth factor receptor) inhibitors, PDGFR (platelet-derived growth factor receptor) inhibitors, MET inhibitors, AXL inhibitors, RET inhibitors, FLT3 (fins-related tyrosine kinase 3) inhibitors, KIT inhibitors, CSF1R (colony-stimulating factor 1 receptor) inhibitors, TIE2 (tunica interna endothelial cell kinase 2) inhibitors, TRKB inhibitors, and CDK4/6 inhibitors. In some embodiments, the ATR inhibitors include AZD6738, berzosertib, BAY1895344, and VX-803. In some embodiments, the Chk1 inhibitors include prexasertib, SCH900776, GDC-0575, and CCT245737. In some embodiments, the HSP90 inhibitors include luminespib, ganetespib, and onalespib. In some embodiments, the PARP inhibitors include olaparib, rucaparib, niraparib, veliparib, and talazoparib. In some embodiments, the EGFR inhibitors include small molecule inhibitors such as lapatinib, gefitinib, erlotinib, afatinib, and vandetanib, and anti-EGFR antibodies such as cetuximab and panitumumab. In some embodiments, the Her2 inhibitors include small molecule inhibitors such as lapatinib, and anti-Her2 antibodies such as trastuzumab, pertuzumab, and trastuzumab emtansine. In some embodiments, the VEGFR inhibitors are inhibitors of at least one of VEGFR1, VEGFR2, and VEGFR3 and include small molecule inhibitors such as sunitinib, cabozantinib, midostaurin, sorafenib, vandetanib, pazopanib, lenvatinib, and axitinib, and anti-VEGFR antibodies such as ramucirumab. In some embodiments, the PDGFR inhibitors are PDGFRα and/or PDGFRβ inhibitors and include sunitinib, midostaurin, pazopanib, lenvatinib, and sorafenib. In some embodiments, the MET inhibitors include cabozantinib, crizotinib, and tepotinib. In some embodiments, the AXL inhibitors include cabozantinib and gilteritinib. In some embodiments, the RET inhibitors include sunitinib, cabozantinib, sorafenib, lenvatinib, and vandetanib. In some embodiments, the FLT3 inhibitors include sunitinib, cabozantinib, midostaurin, gilteritinib, and sorafenib. In some embodiments, the KIT inhibitors include sunitinib, midostaurin, pazopanib, lenvatinib, and sorafenib. In some embodiments, the CSF1R inhibitors include sunitinib, BLZ-945, and ARRY-382. In some embodiments, the TIE2 inhibitors include cabozantinib. In some embodiments, the TRKB inhibitors include cabozantinib and entrectinib. In some embodiments, the CDK4/6 inhibitors include palbociclib, ribociclib, and abemaciclib.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, tautomer, or stereoisomer thereof, is co-administered with a second therapeutic agent, wherein the compound described herein, or a pharmaceutically acceptable salt, solvate, tautomer, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent.

Therapeutically-effective dosages of drugs and other agents for use in combination treatment regimens are optionally determined by means similar to those set forth hereinabove for the actives themselves.

Furthermore, the methods of treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, tautomer, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, tautomer, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, tautomer, or stereoisomer thereof, as well as combination therapies, are administered before, during, or after the occurrence of a disease or condition, and the timing of administering the composition containing a compound varies. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound described herein, or a pharmaceutically acceptable salt, solvate, tautomer, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

### Synthesis of the common Intermediate 5-((1S)-1-amino-2-(6-fluoro-2,3-dimethylphenyl) propyl)-1,3,4-oxadiazol-2 (3H)-one I

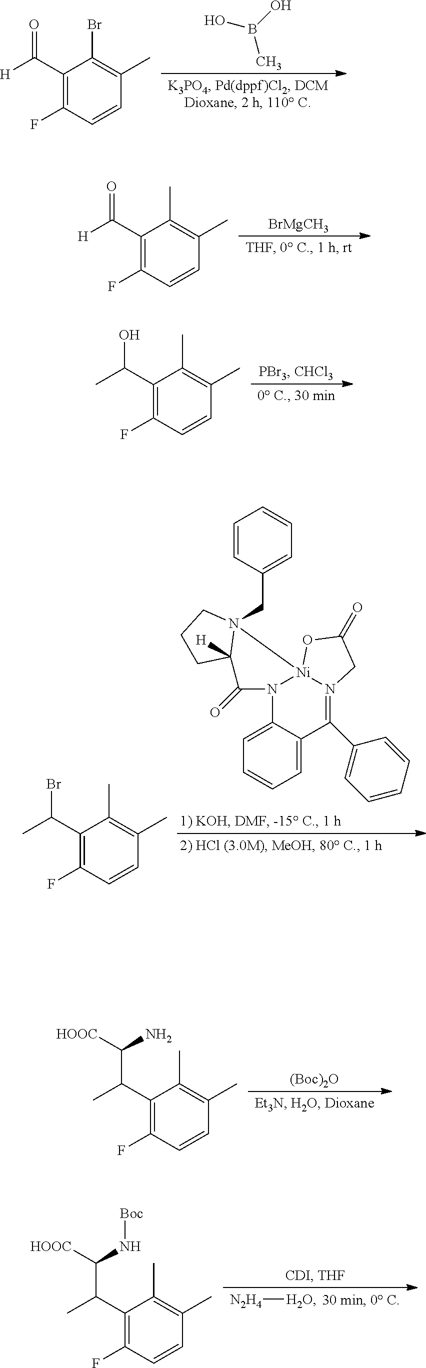

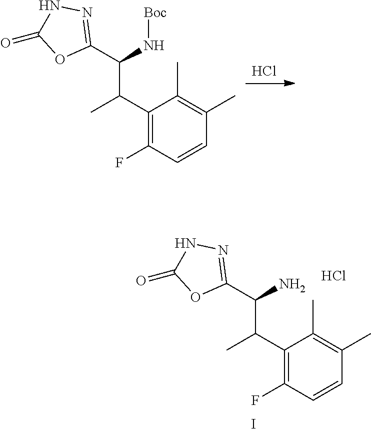

### Step 1. Synthesis of 6-fluoro-2,3-dimethylbenzaldehyde

Into a 1 L round-bottom flask purged and maintained with an inert atmosphere of nitrogen, was placed 2-bromo-6-fluoro-3-methylbenzaldehyde (50 g, 230 mmol, 1.0 equiv), methylboronic acid (23.4 g, 392 mmol, 1.7 equiv), K₃PO₄ (117.4 g, 553 mmol, 2.4 equiv), Pd(dppf)Cl₂·CH₂Cl₂ (5.63 g, 6.91 mmol, 0.03 equiv), H₂O (50 mL), Dioxane (450 mL). The resulting solution was stirred 2 hr at 110° C. The reaction was then quenched by the addition of 200 mL of brine. The resulting solution was extracted with 3×50 mL of ethyl acetate, and the organic layers combined. The residue was applied onto a silica gel column with ethyl acetate/petroleum ether (1:3). This resulted in 6-fluoro-2,3-dimethylbenzaldehyde (30 g, 85%) as a light-yellow oil.

### Step 2. 1-(6-fluoro-2,3-dimethylphenyl) ethan-1-ol

Into a 1 L 3-necked round-bottom flask, the mixture of 6-fluoro-2,3-dimethylbenzaldehyde (27 g, 177.4 mmol, 1 equiv) in THF was added bromo(methyl)magnesium (42.3 g, 355 mmol, 2 equiv) dropwise at 0° C. under nitrogen atmosphere. The resulting mixture was stirred for 2 h at room temperature. The reaction was quenched with sat. NH₄Cl (aq.) at room temperature. The resulting mixture was extracted with EtOAc (3×50 mL). The combined organic layers were washed with brine (2×20 mL), dried over anhydrous MgSO₄. After filtration, the filtrate was concentrated under reduced pressure. to afford 1-(6-fluoro-2,3-dimethylphenyl) ethanol (27 g, 90.5%).

### Step 3. 2-(1-bromoethyl)-1-fluoro-3,4-dimethylbenzene

Into a 500 mL 3-necked round-bottom flask were added 1-(6-fluoro-2,3-dimethylphenyl) ethanol (25 g, 148.6 mmol, 1.0 equiv) and CHCl₃ (250 mL) at room temperature. To the above mixture was added PBr₃ (63.5 mL, 668.8 mmol, 4.5 equiv) dropwise at 0° C. The resulting mixture was stirred for additional 30 min at 0° C. The reaction was quenched by the addition of NaHCO₃ (aq.) (100 mL) at 0° C. The resulting mixture was extracted with CH₂Cl₂ (3×50 mL). The combined organic layers were washed with brine (1×30 mL), dried over anhydrous MgSO₄. After filtration, the filtrate was concentrated under reduced pressure to afford 2-(1-bromoethyl)-1-fluoro-3,4-dimethylbenzene (29 g, 84.4%).

### Step 4. (2S)-2-amino-3-(6-fluoro-2,3-dimethylphenyl) butanoic acid

Into a 500 mL 3-necked round-bottom flask, to a mixture of Ni—(S)—BPB-Gly (5.39 g, 10.8 mmol, 0.5 equiv) in DMF (42.4 mL) was added 2-(1-bromoethyl)-1-fluoro-3,4-dimethylbenzene (5 g, 21.6 mmol, 1.0 equiv) dropwise at room temperature under nitrogen atmosphere. To the resulting mixture was added KOH (6.07 g, 108.2 mmol, 5.0 equiv) in portions at −15° C. under nitrogen atmosphere, stirred for 1 h at −15° C. under nitrogen atmosphere. The reaction was quenched by the addition of sat. NH₄C₁ (aq.) (100 mL) at room temperature, extracted with EtOAc (3×30 mL). The combined organic layers were washed with brine (3×30 mL), dried over anhydrous Na₂SO₄. After filtration, the filtrate was concentrated under reduced pressure, purified by silica gel column chromatography, eluted with PE/EtOAc (1:5). To the mixture were added MeOH (42 mL) and HCl (50 mL) at room temperature. The resulting mixture was stirred for 1 h at 80° C., then purified by reverse phase flash with the following conditions (water:ACN=80:20) to afford (2S)-2-amino-3-(6-fluoro-2,3-dimethylphenyl)butanoic acid (3.05 g, 63%).

### Step 5. (2S)-2-((tert-butoxycarbonyl) amino)-3-(6-fluoro-2,3-dimethylphenyl) butanoic acid

Into a 250 mL round-bottom flask were added (2S)-2-amino-3-(6-fluoro-2,3-dimethylphenyl) butanoic acid (5 g, 22.2 mmol, 1.0 equiv), Et₃N (6.74 g, 66.6 mmol, 3.0 equiv), H₂O (25 mL) and Dioxane (25 mL) at room temperature. To the resulting mixture was added di-tert-butyl dicarbonate (7.27 g, 33.3 mmol, 1.5 equiv) in portions at 0° C. The resulting mixture was stirred for 2 h at room temperature. The crude product was purified by reverse phase flash chromatography with the following conditions (water:ACN 40:60) to afford (2S)-2-[(tert-butoxycarbonyl) amino]-3-(6-fluoro-2,3-dimethylphenyl) butanoic acid (3.5 g, 48.5%).

### Step 6: synthesis of 5-((1S)-1-amino-2-(6-fluoro-2,3-dimethylphenyl) propyl)-1,3,4-oxdiazol-2 (3H)-one, HCl

Into a 250-mL round-bottom flask were added (2S)-2-[(tert-butoxycarbonyl) amino]-3-(6-fluoro-2,3-dimethylphenyl) butanoic acid (9 g, 27.7 mmol, 1.0 equiv), CDI (11.2 g, 69.2 mmol, 2.5 equiv) and THF (60 mL) at room temperature, the resulting mixture was stirred for 30 min at room temperature. To the mixture was added hydrazine (4.15 mL, 82.901 mmol, 3.0 equiv) dropwise at 0° C. The resulting mixture was stirred for 30 min at 0° C. The reaction was quenched with water at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. To the crude mixture was added dioxane (60 mL) and CDI (11.2 g, 69.2 mmol, 2.5 equiv) at room temperature. The resulting mixture was stirred for 30 min at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure, purified by silica gel column chromatography, eluted with PE/EtOAc (2:3) to afford tert-butyl N-[(1S,2R)-2-(6-fluoro-2,3-dimethylphenyl)-1-(5-oxo-4H-1,3,4-oxadiazol-2-yl) propyl]carbamate (3 g, 29.7%) as a light-yellow oil. The product was dissolved in 2 ml THF and treated with 2 ml of HCl 4N in THF. The reaction was left overnight at RT and concentrated under vacuum yielding 5-((1S)-1-amino-2-(6-fluoro-2,3-dimethylphenyl) propyl)-1,3,4-oxadiazol-2 (3H)-one, HCl as off-white solid (2.4 g, Yield 100%).

### Synthesis of the Common Intermediate II: methyl (2S)-2-amino-3-(6-fluoro-2,3-dimethylphenyl) butanoate

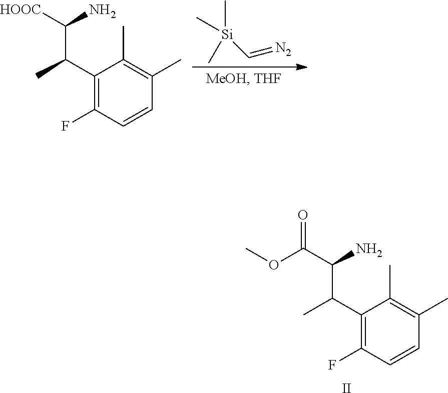

Into a 500 mL 3-necked round-bottom flask were added (2S)-2-amino-3-(6-fluoro-2,3-dimethylphenyl) butanoic acid (12.8 g, 56.8 mmol, 1.00 equiv), trimethylsilyldiazomethane (56.8 mL, 113.6 mmol, 2.0 equiv), MeOH (130 mL) and THF (380 mL) at room temperature. The resulting mixture was stirred for 3 h at room temperature under nitrogen atmosphere. The resulting mixture was concentrated under reduced pressure. The residue was purified by silica gel column chromatography, eluted with PE/EtOAc (1:1) to afford methyl (2S)-2-amino-3-(6-fluoro-2,3-dimethylphenyl) butanoate (9.9 g, 72.8%).

### Synthesis of the Common Intermediate III: tert-butyl (2S)-2-amino-3-(6-fluoro-2,3-dimethylphenyl)

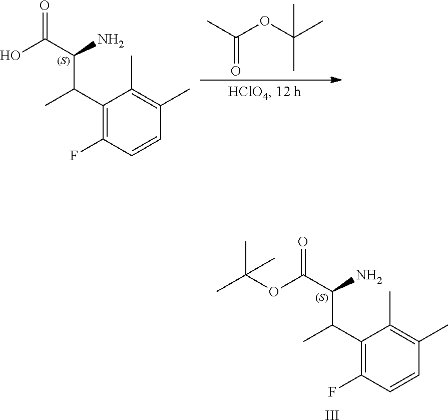

Into a 250 mL round-bottom flask were added (2S)-2-amino-3-(6-fluoro-2,3-dimethylphenyl) butanoic acid (15 g, 67 mmol, 1 equiv) and tert-butyl acetate (160 mL) at room temperature. To the above mixture was added HClO₄ (21 mL, 366 mmol, 5.50 equiv) dropwise at 0° C. The resulting mixture was stirred for additional 1 h at room temperature. The reaction was quenched by the addition of HCl(1M) (240 mL) at room temperature. The mixture was basified to pH9 with Na₂CO₃ (solid) (300 mL). The resulting mixture was extracted with EtOAc (3×300 mL). The combined organic layers were washed with brine (1×300 mL), dried over anhydrous Na₂SO₄. After filtration, the filtrate was concentrated under reduced pressure. The residue was purified by silica gel column chromatography, eluted with PE/EA (1:1) to afford tert-butyl (2S)-2-amino-3-(6-fluoro-2,3-dimethylphenyl) butanoate (12 g, 68.6%).

### Example 1: 5-((1S)-1-(6-chloro-4,4-dimethyl-1,1-dioxido-3,4-dihydro-2H-benzo[e][1,2]thiazin-2-yl)-2-(6-fluoro-2,3-dimethylphenyl)propyl)-1,3,4-oxadiazol-2 (3H)-one

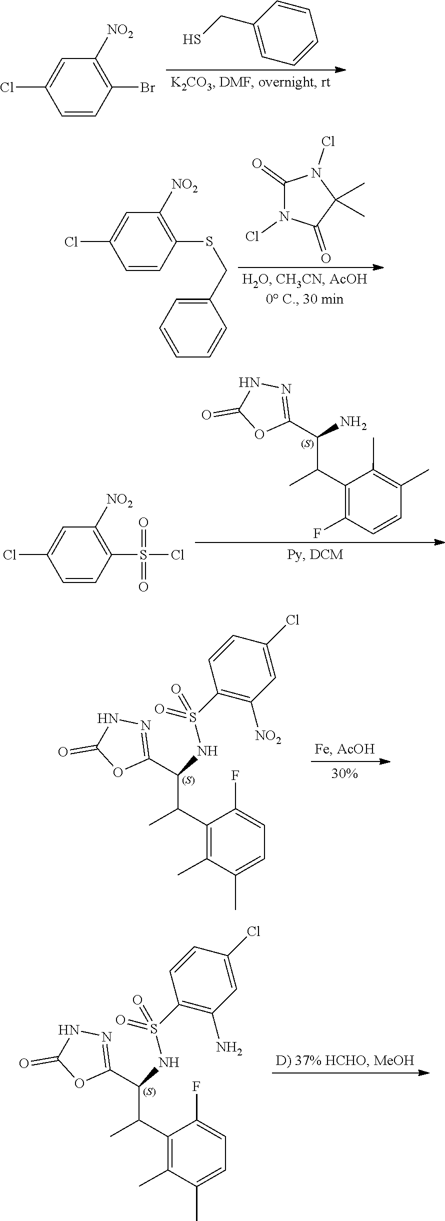

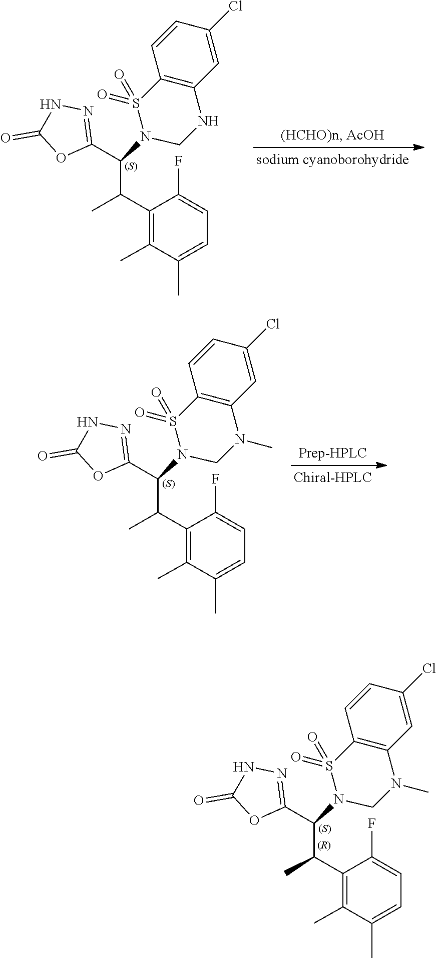

### Step 1: Synthesis of 1-(benzylsulfanyl)-4-chloro-2-nitrobenzene

To a stirred solution/mixture of 1-bromo-4-chloro-2-nitrobenzene (2.5 g, 10.6 mmol, 1 equiv) and DMF (50 mL) was added Cs₂CO₃ (17.2 g, 52.9 mmol, 5.0 equiv) benzyl mercaptan (1.58 g, 12.7 mmol, 1.2 equiv) dropwise at room temperature. The resulting mixture was stirred for overnight at room temperature. The reaction was quenched with water at room temperature. The resulting mixture was extracted with diethylether (3×100 mL). The combined organic layers were washed with brine (3×150 mL), dried over anhydrous Na₂SO₄. After filtration, the filtrate was concentrated under reduced pressure. The crude product was re-crystallized from PE/ethyl acetate (10:1 10 mL) to afford 1-(benzylsulfanyl)-4-chloro-2-nitrobenzene (2 g, 67.6%).

### Step 2: Synthesis of 4-chloro-2-nitrobenzenesulfonyl chloride

To a stirred solution of 1-(benzylsulfanyl)-4-chloro-2-nitrobenzene (1.6 g, 5.7 mmol, 1 equiv) and H₂O (1 mL) in acetonitrile was added AcOH (1.47 mL, 25.7 mmol, 4.5 equiv) in portions at room temperature. To the above mixture was added 1,3-dichloro-5,5-dimethylimidazolidine-2,4-dione (2.25 g, 11.4 mmol, 2 equiv) in portions over 10 min at 0° C. The resulting mixture was stirred 30 min at 0° C. The reaction was quenched with water/ice. The resulting mixture was extracted with EtOAc (3×30 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (10:1) to afford 4-chloro-2-nitrobenzenesulfonyl chloride (1.86 g).

### Step 3: Synthesis of 4-chloro-N-[(1S)-2-(6-fluoro-2,3-dimethylphenyl)-1-(5-oxo-4H-1,3,4-oxadiazol-2-yl) propyl]-2-nitrobenzenesulfonamide

To a stirred solution of 5-[(1S)-1-amino-2-(6-fluoro-2,3-dimethylphenyl) propyl]-3H-1,3,4-oxadiazol-2-one hydrochloride (1.86 g, 7.3 mmol, 1.0 equiv) and pyridine (5 mL) were added 4-chloro-2-nitrobenzenesulfonyl chloride (1.83 g, 6.1 mmol, 1.2 equiv) in DCM (5 mL) dropwise at 0° C. The resulting mixture was stirred overnight at room temperature. The resulting mixture was concentrated under vacuum. The residue was dissolved in DCM (20 mL). The resulting mixture was washed with 3×20 mL of HCl(1M). The residue was purified by silica gel column chromatography, eluted with PE/EtOAc (8:1) to afford 4-chloro-N-[(1S)-2-(6-fluoro-2,3-dimethylphenyl)-1-(5-oxo-4H-1,3,4-oxadiazol-2-yl) propyl]-2-nitrobenzenesulfonamide (1.29 g, 43.9%).

### Step 4: Synthesis of 2-amino-4-chloro-N-[(1S)-2-(6-fluoro-2,3-dimethylphenyl)-1-(5-oxo-4H-1,3,4-oxadiazol-2-yl) propyl]benzenesulfonamide

To a stirred solution of 4-chloro-N-[(1S)-2-(6-fluoro-2,3-dimethylphenyl)-1-(5-oxo-4H-1,3,4-oxadiazol-2-yl) propyl]-2-nitrobenzenesulfonamide (1.29 g, 2.7 mmol, 1 equiv) in AcOH was added Fe (2225 mg, 39.8 mmol, 15 equiv) at room temperature. The resulting mixture was stirred for 30 min at 80° C. The resulting mixture was concentrated under vacuum. The residue was dissolved in DCM (20 mL).

The resulting mixture was washed with 1×20 mL of NaHCO₃ (sat.). The resulting mixture was concentrated under vacuum. The residue was purified by reverse flash chromatography with the following conditions: column, C18 silica gel; mobile phase, MeCN in Water (10 mmol/L NH₄HCO₃), 0% to 100% gradient in 20 min; detector, UV 220 nm. This resulted in 2-amino-4-chloro-N-[(1S)-2-(6-fluoro-2,3-dimethylphenyl)-1-(5-oxo-4H-1,3,4-oxadiazol-2-yl) propyl]benzenesulfonamide (400 mg, 33.1%).

### Step 5: Synthesis of 5-((1S)-1-(6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution of 2-amino-4-chloro-N-[(1S)-2-(6-fluoro-2,3-dimethylphenyl)-1-(5-oxo-4H-1,3,4-oxadiazol-2-yl) propyl]benzenesulfonamide (128 mg, 0.28 mmol, 1 equiv) in 1 ml of MeOH is added 37% HCHO (1332 μL, 3.6 mmol, 12.9 equiv) at room temperature. The resulting mixture was stirred overnight at room temperature. The residue was purified by reverse flash chromatography with the following conditions: column, C18 silica gel; mobile phase, MeCN in Water (10 mmol/L NH₄HCO₃), 0% to 100% gradient in 20 min; detector, UV 254 nm. This resulted in 5-((1S)-1-(6-chloro-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (95 mg, 72.3%).

### Step 6: Synthesis of 5-((1S,2R)-1-(6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution of 5-((1S)-1-(6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (92 mg, 0.2 mmol, 1 equiv) and (HCHO)n (59.1 mg, 1.97 mmol, 10 equiv) was added AcOH (1.84 mL) dropwise at room temperature. The resulting mixture was stirred for 60 min at room temperature. To the above mixture was added NaBH₃CN (37.2 mg, 0.59 mmol, 3 equiv) in portions at room temperature. The resulting mixture was stirred overnight at room temperature. The resulting mixture was concentrated under vacuum. The residue was purified by reverse flash chromatography with the following conditions: column, C18 silica gel; mobile phase, MeCN in Water (10 mmol/L NH₄HCO₃), 0% to 100% gradient in 15 min; detector, UV 220 nm. This resulted in 5-((1S)-1-(6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (85 mg, 89.7%).

The product (85 mg) was further purified by Chiral-Prep-HPLC with the following conditions: Column, XBridge Prep OBD C18 Column, 30*150 mm, 5 μm; mobile phase, Water(10 mmol/L NH₄HCO₃+0.1% NH₃·H₂O) and ACN (25% ACN up to 52% in 8 min); Detector, UV 254 nm. This resulted in 6-chloro-2-[(1S,2S)-2-(6-fluoro-2,3-dimethylphenyl)-1-(5-oxo-4H-1,3,4-oxadiazol-2-yl)propyl]-4-methyl-3H-1lambda6,2,4-benzothiadiazine-1,1-dione (3.6 mg, 4.24%).

LCMS:(ES, m/z):(M−H)=479.1. ¹H NMR (400 MHz, Methanol-d₄) δ7.55 (d, J=8.4 Hz, 1H), 6.99-6.96 (dd, J=8.4, 5.7 Hz, 1H), 6.78-6.72 (dd, J=8.5, 1.9 Hz, 1H), 6.72-6.67 (m, 2H), 5.48-5.41 (m, 1H), 5.35-5.32 (d, J=14.5 Hz, 1H), 5.05-5.02 (d, J=14.5 Hz, 21), 3.92-3.86 (dtd, J=13.0, 7.7, 6.2 Hz, 1H), 2.88 (s, 3H), 2.37 (s, 3H), 2.21 (s, 31), 1.46-1.44 (dd, J=7.0, 1.1 Hz, 3H).

### Example 2: 5-((1S,2R)-1-(7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

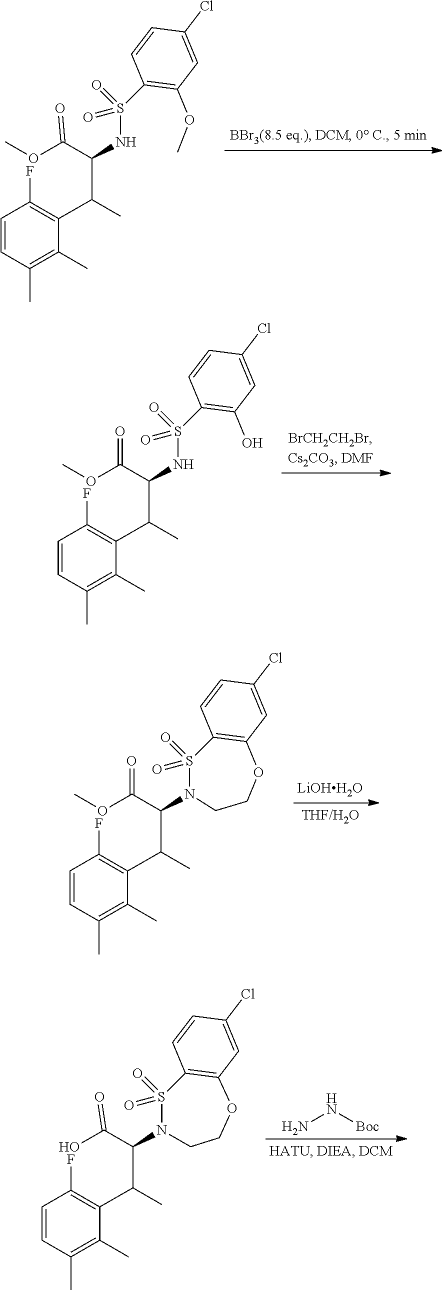

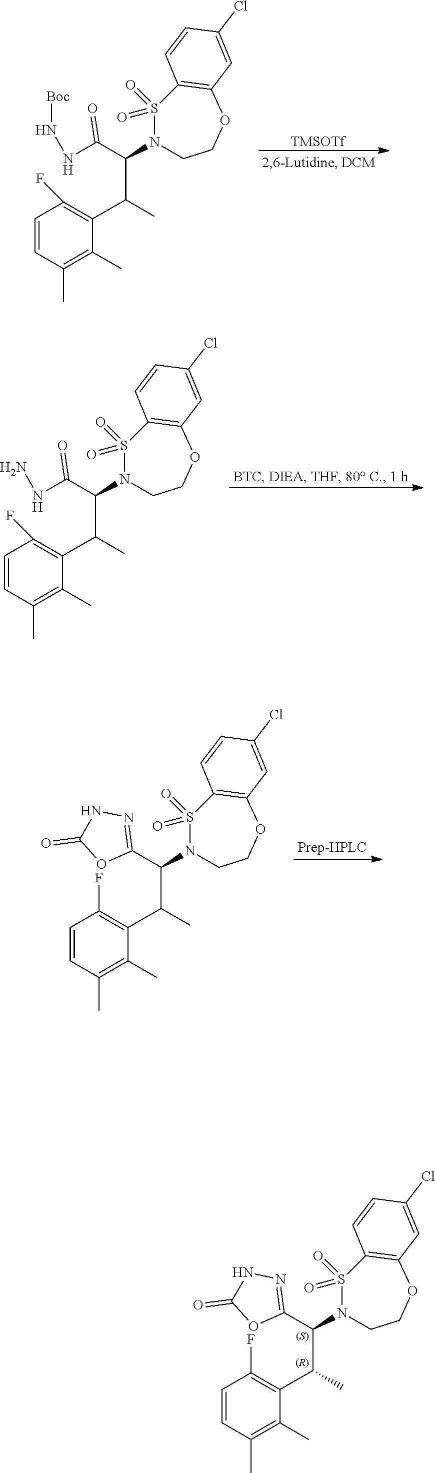

### Step 1: Synthesis of methyl (2S)-2-(4-chloro-2-hydroxybenzenesulfonamido)-3-(6-fluoro-2,3-dimethylphenyl) butanoate

In a 50 mL round-bottom flask were added methyl (2S)-2-(4-chloro-2-methoxybenzenesulfonamido)-3-(6-fluoro-2,3-dimethylphenyl) butanoate (490 mg, 1.1 mmol, 1 equiv) and DCM (5 mL) at room temperature. To the above mixture was added boron tribromide (14.4 mL, 14.3 mmol, 13 equiv) dropwise over 10 min at 0° C. The reaction was quenched with water at 0° C. The resulting mixture was extracted with DCM (1×20 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EtOAc (77:23) to afford methyl (2S)-2-(4-chloro-2-hydroxybenzenesulfonamido)-3-(6-fluoro-2,3-dimethylphenyl) butanoate (340 mg, 71.7%).

### Step 2: Synthesis of methyl (2S)-2-(7-chloro-1,1-dioxido-3,4-dihydro-2H-benzo[b][1,4,5]oxathiazepin-2-yl)-3-(6-fluoro-2,3-dimethylphenyl)butanoate

To a stirred mixture of methyl (2S)-2-(4-chloro-2-hydroxybenzenesulfonamido)-3-(6-fluoro-2,3-dimethylphenyl) butanoate (340 mg, 0.79 mmol, 1 equiv) and DMF (5 mL) were added Cs₂CO₃ (773 mg, 2.37 mmol, 3 equiv) and dibromoethane (68 μL, 0.79 mmol, 1 equiv) in portions at room temperature. The resulting mixture was stirred overnight at 60° C. The reaction was quenched with water at room temperature. The resulting mixture was extracted with EtOAc (3×5 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (80:20) to afford methyl (2S)-2-(7-chloro-1,1-dioxido-3,4-dihydro-2H-benzo[b][1,4,5] oxathiazepin-2-yl)-3-(6-fluoro-2,3-dimethylphenyl)butanoate (290 mg, 80.4%).

### Step 3: Synthesis of (2S)-2-(7-chloro-1,1-dioxido-3,4-dihydro-2H-benzo[b][1,4,5]oxathiazepin-2-yl)-3-(6-fluoro-2,3-dimethylphenyl)butanoic acid

To a stirred mixture of methyl (2S)-2-(7-chloro-1,1-dioxido-3,4-dihydro-2H-benzo[b][1,4,5]oxathiazepin-2-yl)-3-(6-fluoro-2,3-dimethylphenyl)butanoate (290 mg, 0.64 mmol, 1 equiv) in MeOH (3 mL) were added NaOH (50.9 mg, 1.27 mmol, 2 equiv) and H₂O (600 μL) dropwise at room temperature under air atmosphere. The resulting mixture was stirred 2 h at 70° C. The mixture was neutralized to pH 5 with HCl (aq.). The resulting mixture was extracted with EtOAc (3×30 mL). The combined organic layers were washed with brine (1×30 mL), dried over anhydrous Na₂SO₄. After filtration, the filtrate was concentrated under reduced pressure. This resulted in (2S)-2-(7-chloro-1,1-dioxido-3,4-dihydro-2H-benzo[b][1,4,5]oxathiazepin-2-yl)-3-(6-fluoro-2,3-dimethylphenyl) butanoic acid (284 mg, 97.00%).

### Step 4: Synthesis of tert-butyl 2-((2S)-2-(7-chloro-1,1-dioxido-3,4-dihydro-2H-benzo[b][1,4,5]oxathiazepin-2-yl)-3-(6-fluoro-2,3-dimethylphenyl)butanoyl)hydrazine-1-carboxylate

To a stirred mixture of (2S)-2-(7-chloro-1,1-dioxido-3,4-dihydro-2H-benzo[b][1,4,5]oxathiazepin-2-yl)-3-(6-fluoro-2,3-dimethylphenyl)butanoic acid (284 mg, 0.64 mmol, 1 equiv) and DIEA (335 μL, 1.93 mmol, 3 equiv) in DCM (2 mL) were added HATU (367 mg, 0.97 mmol, 1.5 equiv) and tert-butoxycarbohydrazide (127 mg, 0.97 mmol, 1.5 equiv) in portions at room temperature. The resulting mixture was stirred 1 h at room temperature. The residue was purified by silica gel column chromatography, eluted with PE/EtOAc (85:15) to afford tert-butyl 2-((2S)-2-(7-chloro-1,1-dioxido-3,4-dihydro-2H-benzo[b][1,4,5]oxathiazepin-2-yl)-3-(6-fluoro-2,3-dimethylphenyl)butanoyl)hydrazine-1-carboxylate (250 mg, 70%).

### Step 5: Synthesis of (2S)-2-(7-chloro-1,1-dioxido-3,4-dihydro-2H-benzo[b][1,4,5]oxathiazepin-2-yl)-3-(6-fluoro-2,3-dimethylphenyl)butanehydrazide

In a 50 mL round-bottom flask were added tert-butyl 2-((2S)-2-(7-chloro-1,1-dioxido-3,4-dihydro-2H-benzo[b][1,4,5]oxathiazepin-2-yl)-3-(6-fluoro-2,3-dimethylphenyl)butanoyl)hydrazine-1-carboxylate (250 mg, 0.45 mmol, 1 equiv) and DCM (3 mL) at room temperature. To the above mixture was added 2,6-Lutidine (1.05 mL, 9.0 mmol, 20 equiv) and trimethylsilyl triflate (469 μL, 2.59 mmol, 16 equiv) dropwise at 0° C. The resulting mixture was stirred for additional 1 h at room temperature. The resulting mixture was concentrated under reduced pressure. The residue was purified by reverse flash chromatography with the following conditions: column, C18 silica gel; mobile phase, MeCN in water, 20% to 80% gradient in 20 min; detector, UV 254 nm. This resulted in (2S)-2-(7-chloro-1,1-dioxido-3,4-dihydro-2H-benzo[b][1,4,5]oxathiazepin-2-yl)-3-(6-fluoro-2,3-dimethylphenyl)butanehydrazide (150 mg, 73.2%).

### Step 6: Synthesis of 5-((1S,2R)-1-(7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

To a stirred mixture of (2S)-2-(7-chloro-1,1-dioxido-3,4-dihydro-2H-benzo[b][1,4,5] oxathiazepin-2-yl)-3-(6-fluoro-2,3-dimethylphenyl)butanehydrazide (150 mg, 0.33 mmol, 1 equiv) in THF (2 mL) were added DIEA (143 μL, 0.82 mmol, 2.5 equiv) and Triphosgene (48.8 mg, 0.17 mmol, 0.5 equiv) in portions at room temperature under air atmosphere. The resulting mixture was stirred r 1 h at 80° C. The resulting mixture was extracted with EtOAc (2×5 mL). The combined organic layers were washed with brine (1×10 mL), dried over anhydrous Na₂SO₄. After filtration, the filtrate was concentrated under reduced pressure. This resulted in of 5-((1S,2R)-1-(7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (150 mg, 94.6%).

The product (150 mg) was further purified by Prep-HPLC with the following conditions (Column: XBridge Prep OBD C18 Column, 19*250 mm, 5 μm; Mobile Phase A: Water (10 mmol/L NH₄HCO₃+0.1% NH₃·H₂O), Mobile Phase B: MeOH-HPLC; Flow rate: 20 mL/min; Gradient: 20% B to 50% B in 8 min, 50% B; Wave Length: 254 nm; RT1 (min): 7.45) to afford 7-chloro-2-[(1S,2R)-2-(6-fluoro-2,3-dimethylphenyl)-1-(5-oxo-4H-1,3,4-oxadiazol-2-yl)propyl]-3,4-dihydro-5,1lambda6,2-benzoxathiazepine-1,1-dione (28.1 mg, 17.82%). LC-MS:(ES, m/z): M−H=480.10. ¹H NMR (400 MHz, Methanol-d₄) δ 7.76 (d, J=8.5 Hz, 1H), 7.27-7.24 (dd, J=8.5, 2.0 Hz, 1H), 7.12 (d, J=2.1 Hz, 1H), 7.01-6.97 (dd, J=8.4, 5.7 Hz, 1H), 6.75-6.70 (dd, J=12.1, 8.4 Hz, 1H), 5.53-5.50 (dd, J=11.6, 2.0 Hz, 1H), 4.64-4.58 (dt, J=13.2, 5.4 Hz, 1H), 4.04-3.92 (d, J=13.4 Hz, 1H), 3.88-3.83 (m, 3H), 2.34 (s, 3H), 2.21 (s, 3H), 1.44 (dd, J=6.9, 1.1 Hz, 3H).

### Example 3: 5-((1S,2R)-1-(6-chloro-4-methyl-1,1-dioxido-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

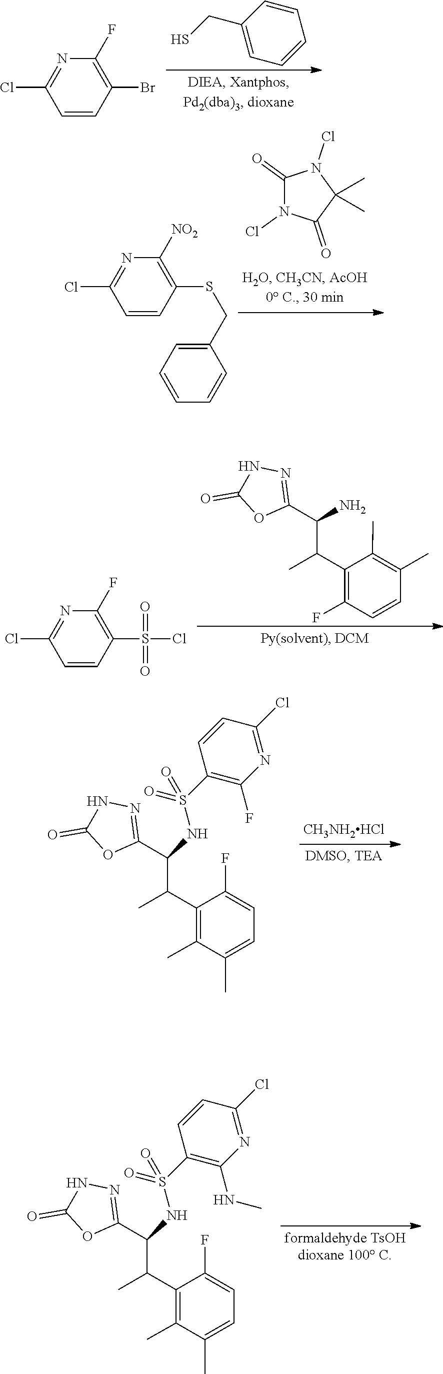

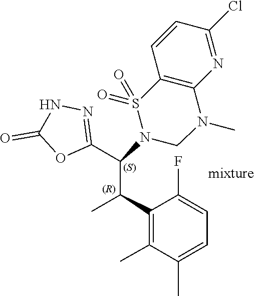

### Step 1: Synthesis of 3-(benzylsulfanyl)-6-chloro-2-fluoropyridine

In a 20 mL round-bottom flask were added 3-bromo-6-chloro-2-fluoropyridine (500 mg, 2.38 mmol, 1 equiv), DIEA (921, 7.13 mmol, 3.0 equiv), Xantphos (275 mg, 0.48 mmol, 0.2 equiv), Pd₂(dba)₃ (218 mg, 0.24 mmol, 0.1 equiv), dioxane (5 mL) and benzyl mercaptan (325 mg, 2.61 mmol, 1.1 equiv) at room temperature. The resulting mixture was stirred 1 h at 80° C. under nitrogen atmosphere. The resulting mixture was filtered, the filter cake was washed with EtOAc (3×10 mL). The filtrate was concentrated under reduced pressure. The residue was purified by silica gel column chromatography, eluted with PE/EtOAc (12:1) to afford 3-(benzylsulfanyl)-6-chloro-2-fluoropyridine (300 mg, 49.8%).

### Step 2: Synthesis of 6-chloro-2-fluoropyridine-3-sulfonyl

Into a 20 mL round-bottom flask were added 3-(benzylsulfanyl)-6-chloro-2-fluoropyridine (1 g, 3.9 mmol, 1 equiv), H₂O (500 μl), AcOH (700 μl) and ACN (10 mL). To the mixture was added 1,3-dichloro-5,5-dimethylimidazolidine-2,4-dione (1.55 g, 7.9 mmol, 2.0 equiv) at 0° C. The resulting mixture was stirred for 30 min at 0° C. under air atmosphere. The resulting mixture was quenched with water and extracted with EtOAc (3×20 mL). The combined organic layers were washed with brine (2×15 mL), dried over anhydrous Na₂SO₄. After filtration, the filtrate was concentrated under reduced pressure to afford 6-chloro-2-fluoropyridine-3-sulfonyl chloride (0.8 g, 88.2%).

### Step 3: Synthesis of 6-chloro-2-fluoro-N-[(1S)-2-(6-fluoro-2,3-dimethylphenyl)-1-(5-oxo-4H-1,3,4-oxadiazol-2-yl) propyl]pyridine-3-sulfonamide

In a 8 mL round-bottom flask were added 5-[(1S)-1-amino-2-(6-fluoro-2,3-dimethylphenyl) propyl]-3H-1,3,4-oxadiazol-2-one (200 mg, 0.75 mmol, 1 equiv) and Pyridine (2 mL) at room temperature. Then were added 6-chloropyridine-3-sulfonyl chloride (239 mg, 1.13 mmol, 1.5 equiv) in DCM (0.5 mL) at 0° C. The resulting mixture was stirred 2h at room temperature under air atmosphere. The resulting mixture was quenched with water and extracted with EtOAc (3×20 mL). The combined organic layers were washed with brine (2×15 mL), dried over anhydrous Na₂SO₄. After filtration, the filtrate was concentrated under reduced pressure to afford 6-chloro-2-fluoro-N-[(1S)-2-(6-fluoro-2,3-dimethylphenyl)-1-(5-oxo-4H-1,3,4-oxadiazol-2-yl) propyl]pyridine-3-sulfonamide (300 mg, 86.7%).

### Step 4: Synthesis of 6-chloro-N-[(1S)-2-(6-fluoro-2,3-dimethylphenyl)-1-(5-oxo-4H-1,3,4-oxadiazol-2-yl) propyl]-2-(methylamino) pyridine-3-sulfonamide

Into a 40 mL round-bottom flask were added 6-chloro-2-fluoro-N-[(1S)-2-(6-fluoro-2,3-dimethylphenyl)-1-(5-oxo-4H-1,3,4-oxadiazol-2-yl) propyl]pyridine-3-sulfonamide (800 mg, 1.74 mmol, 1 equiv), methylamine hydrochloride (1.65 g, 24.4 mmol, 14 equiv), TEA (2470 mg, 24.4 mmol, 14 equiv) and DMSO (10 mL) at room temperature. The resulting mixture was stirred overnight at room temperature under air atmosphere. The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 5% to 100% gradient in 30 min; detector, UV 254 nm, to afford 6-chloro-N-[(1S)-2-(6-fluoro-2,3-dimethylphenyl)-1-(5-oxo-4H-1,3,4-oxadiazol-2-yl)propyl]-2-(methylamino)pyridine-3-sulfonamide (200 mg, 24.4%).

### Step 5: Synthesis of 5-((1S,2R)-1-(6-chloro-4-methyl-1,1-dioxido-3,4-dihydro-2H-pyrido[2,3-e][1,2,4]thiadiazin-2-yl)-2-(6-fluoro-2,3-dimethylphenyl) propyl)-1,3,4-oxadiazol-2 (3H)-one

Into a 40 mL round-bottom flask were added 6-chloro-N-[(1S)-2-(6-fluoro-2,3-dimethylphenyl)-1-(5-oxo-4H-1,3,4-oxadiazol-2-yl) propyl]-2-(methylamino) pyridine-3-sulfonamide (120 mg, 0.26 mmol, 1 equiv), TsOH (44 mg, 0.26 mmol, 1.0 equiv), Paraformaldehyde (230 mg, 2.6 mmol, 10 equiv) and dioxane (2 mL) at room temperature. The resulting mixture was stirred overnight at 100° C. under nitrogen atmosphere. The resulting mixture was concentrated under reduced pressure. The residue was purified by reverse flash chromatography with the following conditions: column, C18 silica gel; mobile phase, MeCN in Water (10 mmol/L NH₄HCO₃), 5% to 100% gradient in 30 min; detector, UV 220 nm. The crude product was purified by Chiral-Prep-HPLC with the following conditions:Column, XBridge Prep OBD C18 Column, 30*150 mm, 5 μm; mobile phase, Water(10 mmol/L NH₄HCO₃+0.1% NH₃·H₂O) and ACN (20% ACN up to 50% in 8 min); Detector, uv 220 nm to afford 6-chloro-2-[(1S,2R)-2-(6-fluoro-2,3-dimethylphenyl)-1-(5-oxo-4H-1,3,4-oxadiazol-2-yl)propyl]-4-methyl-3H-1lambda6-pyrido[2,3-e][1,2,4]thiadiazine-1,1-dione (17.7 mg, 14.35%). LCMS:(ES, m/z): M−H:480.10. ¹H NMR (400 MHz, Methanol-cd) S 7.88 (d, J=8.0 Hz, 1H), 7.00 (dd, J=8.4, 5.8 Hz, 1H), 6.78-6.67 (m, 2H), 5.47-5.27 (m, 2H), 5.13 (d, J=14.8 Hz, 1H), 3.91 (dq, J=13.5, 6.8 Hz, 1H), 3.02 (s, 3H), 2.38 (s, 3H), 2.22 (s, 3H), 1.45 (d, J=6.9 Hz, 3H).

### Example 4: 5-((1S,2R)-1-(6-chloro-4-isopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

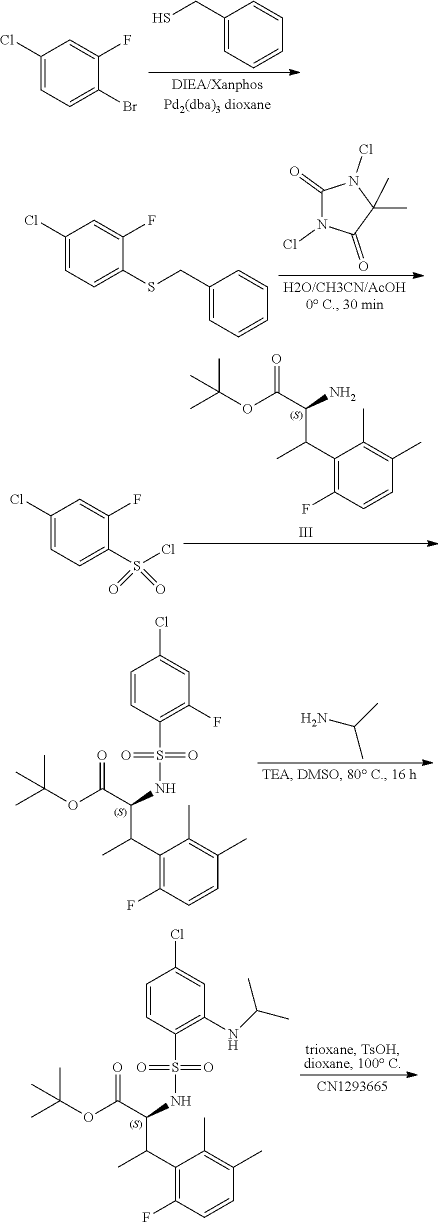

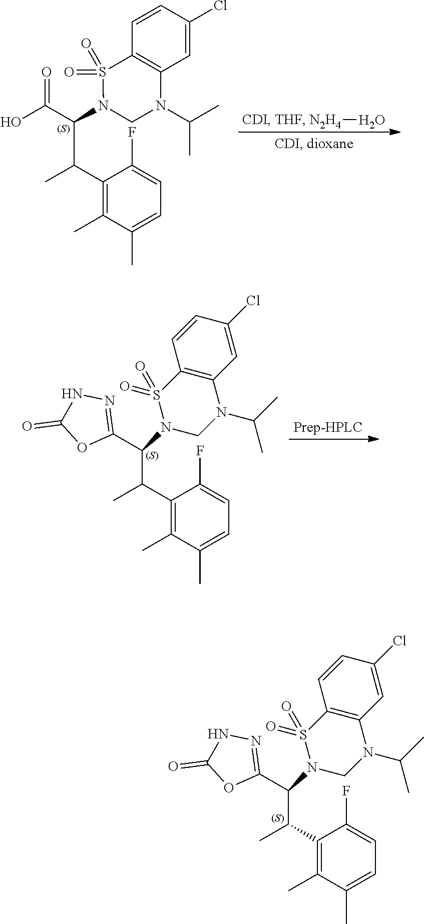

### Step 1: Synthesis of 1-(benzylsulfanyl)-4-chloro-2-fluorobenzene

To a stirred mixture of 1-bromo-4-chloro-2-fluorobenzene (1 g, 4.78 mmol, 1 equiv) and DIEA (1.85 g, 14.3 mmol, 3 equiv) in dioxane (10 mL) were added Xantphos (553 mg, 0.96 mmol, 0.2 equiv) and Pd₂(dba)₃ (437 mg, 0.48 mmol, 0.1 equiv) and benzyl mercaptan (593 mg, 4.78 mmol, 1 equiv) in portions at room temperature under nitrogen atmosphere. The resulting mixture was stirred overnight at 100° C. under nitrogen atmosphere. The resulting mixture was filtered, the filter cake was washed with ethyl acetate (3×10 mL). The filtrate was concentrated under reduced pressure. The residue was purified by silica gel column chromatography, eluted with PE/EtOAc (12:1) to afford 1-(benzylsulfanyl)-4-chloro-2-fluorobenzene (1 g, 82.9%).

### Step 2: Synthesis of 4-chloro-2-fluorobenzenesulfonyl chloride

To a stirred mixture of 1-(benzylsulfanyl)-4-chloro-2-fluorobenzene (1.47 g, 5.8 mmol, 1 equiv) in ACN (15 mL) were added AcOH (0.8 mL) and H₂O (0.8 mL). To the mixture was added 1,3-dichloro-5,5-dimethylimidazolidine-2,4-dione (2.29 g, 11.6 mmol, 2 equiv) in portions at 0° C. under air atmosphere. The resulting mixture was stirred for 30 min at 0° C. under nitrogen atmosphere. The resulting mixture was quenched with water and extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EtOAc (10:1) to afford 4-chloro-2-fluorobenzenesulfonyl chloride (1.14 g, 85.6%).

### Step 3: Synthesis of tert-butyl (2S)-2-(4-chloro-2-fluorobenzenesulfonamido)-3-(6-fluoro-2,3-dimethylphenyl) butanoate

Into a 250 mL round-bottom flask were added tert-butyl (2S)-2-amino-3-(6-fluoro-2,3-dimethylphenyl) butanoate (III) (1.96 g, 6.96 mmol, 1 equiv), DCM (13.8 mL), pyridine (1.9 mL, 24 mmol, 5 equiv). To the mixture was added 4-chloro-2-fluorobenzenesulfonyl chloride (1.10 g, 4.8 mmol, 1.0 equiv) at 0° C. The resulting mixture was stirred overnight at room temperature. The resulting mixture was concentrated under reduced pressure. The residue was purified by silica gel column chromatography, eluted with PE/EtOAc (6:1) to afford tert-butyl (2S)-2-(4-chloro-2-fluorobenzenesulfonamido)-3-(6-fluoro-2,3-dimethylphenyl)butanoate (1.78 g, 78.3%).

### Step 4: Synthesis of tert-butyl (2S)-2-[4-chloro-2-(isopropylamino) benzenesulfonamido]-3-(6-fluoro-2,3-dimethylphenyl) butanoate

To a stirred solution of tert-butyl (2S)-2-(4-chloro-2-fluorobenzenesulfonamido)-3-(6-fluoro-2,3-dimethylphenyl) butanoate (350 mg, 0.74 mmol, 1 equiv) in DMSO (4 mL) were added TEA (1.44 mL, 10.3 mmol, 14 equiv) and isopropylamine (888 μL, 10.3 mmol, 14 equiv) at room temperature. The resulting mixture was stirred for overnight at 80° C. The residue was purified by reverse flash chromatography with the following conditions: column, C18 silica gel; mobile phase, MeCN in Water (0.1% FA), 10% to 100% gradient in 10 min; detector, UV 254 nm. This resulted in tert-butyl (2S)-2-[4-chloro-2-(isopropylamino) benzenesulfonamido]-3-(6-fluoro-2,3-dimethylphenyl) butanoate (370 mg, 97.6%).

### Step 5: Synthesis of (2S)-2-(6-chloro-4-isopropyl-1,1-dioxido-3,4-dihydro-2H-benzo[e][1,2,4]thiadiazin-2-yl)-3-(6-fluoro-2,3-dimethylphenyl)butanoic acid

To a stirred solution of tert-butyl (2S)-2-[4-chloro-2-(isopropylamino) benzenesulfonamido]-3-(6-fluoro-2,3-dimethylphenyl) butanoate (370 mg, 0.72 mmol, 1 equiv) in dioxane (14.8 mL) were added TsOH (124 mg, 0.72 mmol, 1 equiv) and 1,3,5-trioxane (650 mg, 7.21 mmol, 10 equiv) in portions at room temperature. The resulting mixture was stirred 24 h at 110° C. The residue was purified by reverse flash chromatography with the following conditions: column, C18 silica gel; mobile phase, MeCN in Water (0.1% FA), 10% to 60% gradient in 10 min; detector, UV 254 nm. This resulted in ((2S)-2-(6-chloro-4-isopropyl-1,1-dioxido-3,4-dihydro-2H-benzo[e][1,2,4]thiadiazin-2-yl)-3-(6-fluoro-2,3-dimethylphenyl)butanoic acid (96 mg, 28.4%).

### Step 6: Synthesis of 5-((1S)-1-(6-chloro-4-isopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

A solution of ((2S)-2-(6-chloro-4-isopropyl-1,1-dioxido-3,4-dihydro-2H-benzo[e][1,2,4]thiadiazin-2-yl)-3-(6-fluoro-2,3-dimethylphenyl)butanoic acid (86 mg, 0.18 mmol, 1 equiv) in THF (1 mL) was treated with CDI (89.2 mg, 0.55 mmol, 3 equiv) for 30 min at room temperature followed by the addition of N₂H₄·H₂O (26.7 μL, 0.55 mmol, 3 equiv) dropwise at 0° C. The resulting mixture was stirred for 30 min at 0° C. The resulting mixture was extracted with EtOAc (2×1 mL). The combined organic layers were dried over anhydrous Na₂SO₄. After filtration, the filtrate was concentrated under reduced. The residue was dissolved in dioxane (1 mL) at room temperature. To the above mixture was added CDI (148.7 mg, 0.92 mmol, 5 equiv) in portions at room temperature. The resulting mixture was stirred for additional 30 min at room temperature. The resulting mixture was concentrated under vacuum. The crude product was used in the next step directly without further purification.

### Step 7: Synthesis of 5-((1S,2R)-1-(6-chloro-4-isopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The crude product (90 mg) was purified by Prep-HPLC with the following conditions (Column: XselectCSH C18 OBDColumn 30*150 mm 5 μm, n; Mobile Phase A: Water(0.1% FA), Mobile Phase B: ACN; Flow rate: 60 mL/min; Gradient: 10% B to 40% B in 8 min, 40% B; Wave Length: 254 nm; RTI(min): 7) to afford 5-((1S,2R)-1-(6-chloro-4-isopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (25.5 mg, 25.6%). LC-MS: (ES, m/z): [M+H]⁺=509.15. ¹H NMR (300 MHz, Methanol-d₄) δ 7.56-7.54 (d, J=8.4 Hz, 1H), 7.01-6.93 (m, 2H), 6.78-6.68 (m, 2H), 5.43-5.39 (dd, J=11.8, 1.8 Hz, 1H), 5.15 (s, 2H), 4.19-4.09 (hept, J=6.8 Hz, 1H), 3.99-3.90 (dddd, J=14.5, 12.9, 8.5, 6.7 Hz, 1H), 2.36 (s, 3H), 2.21 (s, 3H), 1.53-1.38 (m, 6H), 1.27 (d, J=6.6 Hz, 3H).

### Example 5: 5-((1S,2R)-1-(6-chloro-4-methyl-1,1-dioxido-3,4-dihydro-2H-pyrido[3,2-e][1,2,4]thiadiazin-2-yl)-2-(6-fluoro-2,3-dimethylphenyl) propyl)-1,3,4-oxadiazol-2 (3H)-one

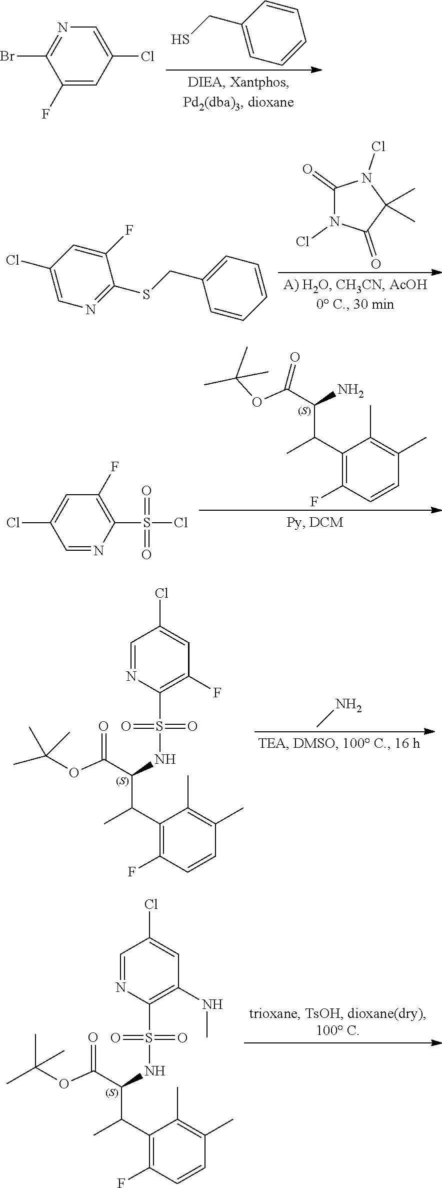

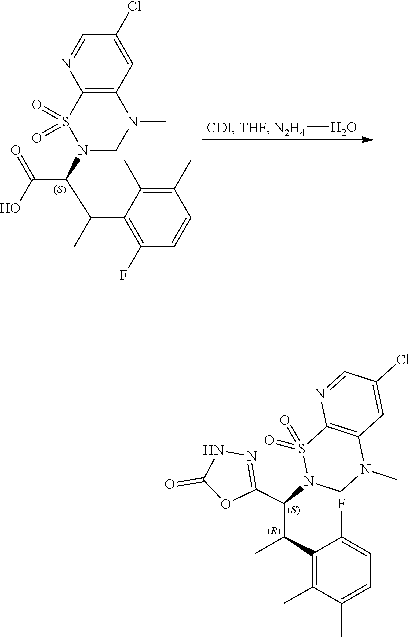

Step 1: Synthesis of 2-(benzylsulfanyl)-5-chloro-3-fluoropyridine

Into a 40 mL round-bottom flask were added 2-bromo-5-chloro-3-fluoropyridine (550 mg, 2.61 mmol, 1 equiv), benzylmercaptan (357 mg, 2.88 mmol, 1.1 equiv), DIEA (1013 mg, 7.84 mmol, 3.0 equiv), Xantphos (302 mg, 0.52 mmol, 0.2 equiv), and Pd₂(dba)₃ (239 mg, 0.26 mmol, 0.1 equiv), dioxane (6 mL) at room temperature. The resulting mixture was stirred overnight at room temperature under nitrogen atmosphere. The resulting mixture was filtered, the filter cake was washed with DCM (3×10 mL). The filtrate was concentrated under reduced pressure. The residue was purified by silica gel column chromatography, eluted with PE/EtOAc (10:1) to afford 2-(benzylsulfanyl)-5-chloro-3-fluoropyridine (450 mg, 67.9%).

### Step 2: Synthesis of 5-chloro-3-fluoropyridine-2-sulfonyl chloride

Into a 20 mL round-bottom flask were added 2-(benzylsulfanyl)-5-chloro-3-fluoropyridine (700 mg, 2.76 mmol, 1 equiv), AcOH (2.45 ml), H₂O (1.75 ml), ACN (7.00 mL). To the mixture was added 1,3-dichloro-5,5-dimethylimidazolidine-2,4-dione (1087 mg, 5.52 mmol, 2.0 equiv) at 0° C. The resulting mixture was stirred for 30 min at 0° C. under air atmosphere. The resulting mixture was quenched with water and extracted with EtOAc (3×10 mL). The combined organic layers were washed with brine (1×10 mL), dried over anhydrous Na₂SO₄. After filtration, the filtrate was concentrated under reduced pressure, to afford 5-chloro-3-fluoropyridine-2-sulfonyl chloride (500 mg, 78.8%) as a light-yellow oil.

### Step 3: Synthesis of tert-butyl (2S)-2-(5-chloro-3-fluoropyridine-2-sulfonamido)-3-(6-fluoro-2,3-dimethylphenyl) butanoate

Into a 20 mL round-bottom flask were added tert-butyl (2S)-2-amino-3-(6-fluoro-2,3-dimethylphenyl) butanoate (20 mg, 0.071 mmol, 1 equiv) and Pyridine (10 mL). To the mixture was added 5-chloro-3-fluoropyridine-2-sulfonyl chloride (1.23 g, 5.33 mmol, 1.5 equiv) in DCM (2 mL) at 0° C. The resulting mixture was stirred for overnight at room temperature under air atmosphere. The residue was purified by reverse flash chromatography with the following conditions: column, C18 silica gel; mobile phase, MeCN in Water (0.1% FA), 5% to 100% gradient in 30 min; detector, UV 220 nm to afford tert-butyl (2S)-2-(5-chloro-3-fluoropyridine-2-sulfonamido)-3-(6-fluoro-2,3-dimethylphenyl) butanoate (700 mg, 41.5%) as a light-yellow oil.

### Step 4: Synthesis of tert-butyl (2S)-2-[5-chloro-3-(methylamino) pyridine-2-sulfonamido]-3-(6-fluoro-2,3-dimethylphenyl) butanoate

Into a 20 mL round-bottom flask were added tert-butyl (2S)-2-(5-chloro-3-fluoropyridine-2-sulfonamido)-3-(6-fluoro-2,3-dimethylphenyl) butanoate (200 mg, 0.42 mmol, 1 equiv), Methylamine (2M in THF) (130.8 mg, 4.21 mmol, 10 equiv) and TEA (426 mg, 4.21 mmol, 10 equiv) in DMSO at room temperature. The resulting mixture was stirred overnight at 65° C. under air atmosphere. The residue was purified by reverse flash chromatography with the following conditions: column, C18 silica gel; mobile phase, MeCN in Water (0.1% FA), 5% to 100% gradient in 30 min; detector, UV 220 nm, to afford tert-butyl (2S)-2-[5-chloro-3-(methylamino) pyridine-2-sulfonamido]-3-(6-fluoro-2,3-dimethylphenyl) butanoate (180 mg, 88%) as a light-yellow solid.

### Step 5: Synthesis of (2S)-2-(6-chloro-4-methyl-1,1-dioxido-3,4-dihydro-2H-pyrido[3,2-e][1,2,4]thiadiazin-2-yl)-3-(6-fluoro-2,3-dimethylphenyl)butanoic acid

Into a 20 mL round-bottom flask were added tert-butyl (2S)-2-[5-chloro-3-(methylamino) pyridine-2-sulfonamido]-3-(6-fluoro-2,3-dimethylphenyl) butanoate (150 mg, 0.31 mmol, 1 equiv), Paraformaldehyde (556 mg, 6.17 mmol, 10 equiv), TsOH (159 mg, 0.93 mmol, 1.5 equiv) and dioxane (3 mL) at room temperature. The resulting mixture was stirred for overnight at 100° C. under air atmosphere. The residue was purified by reverse flash chromatography with the following conditions: column, C18 silica gel; mobile phase, MeCN in Water (0.1% FA), 5% to 100% gradient in 30 min; detector, UV 254 nm, to afford (2S)-2-(6-chloro-4-methyl-1,1-dioxido-3,4-dihydro-2H-pyrido[3,2-e][1,2,4]thiadiazin-2-yl)-3-(6-fluoro-2,3-dimethylphenyl)butanoic acid (100 mg, 65.1%) as a light yellow oil.

### Step 6: Synthesis of 5-((1S,2R)-1-(6-chloro-4-methyl-1,1-dioxido-3,4-dihydro-2H-pyrido[3,2-e][1,2,4]thiadiazin-2-yl)-2-(6-fluoro-2,3-dimethylphenyl)propyl)-1,3,4-oxadiazol-2 (3H)-one

Into a 50 mL round-bottom flask were added (2S)-2-(6-chloro-4-methyl-1,1-dioxido-3,4-dihydro-2H-pyrido[3,2-e][1,2,4]thiadiazin-2-yl)-3-(6-fluoro-2,3-dimethylphenyl)butanoic acid (200 mg, 0.44 mmol, 1 equiv), CDI (142 mg, 0.88 mmol, 2.0 equiv) and THF (3 mL) at room temperature, the resulting mixture was stirred for 30 min at room temperature. the mixture was added hydrazine (42 mg, 1.32 mmol, 3.0 equiv) dropwise at 0° C. The resulting mixture was stirred for 30 min at 0° C. The reaction was quenched with water at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure, added dioxane (5 mL) and CDI (185 mg, 1.14 mmol, 2.6 equiv) at room temperature. The resulting mixture was stirred for 30 min at room temperature. The resulting mixture was extracted with EtOAc (3×20 mL). The combined organic layers were washed with brine (2×10 mL), dried over anhydrous Na₂SO₄. The residue was purified by reverse flash chromatography with the following conditions: column, C18 silica gel; mobile phase, MeCN in Water (0.1% FA), 5% to 100% gradient in 30 min; detector, UV 220 nm, the crude product was purified by Chiral-Prep-HPLC with the following conditions:Column, Xselect CSH F-Phenyl OBD column, 19*250 mm, 5 μm; mobile phase, Water(0.05% FA) and ACN (44% ACN up to 53% in 11 min); Detector, UV 220 to afford 6-chloro-2-[(1S,2R)-2-(6-fluoro-2,3-dimethylphenyl)-1-(5-oxo-4H-1,3,4-oxadiazol-2-yl)propyl]-4-methyl-3H-1lambda6-pyrido[3,2-e][1,2,4]thiadiazine-1,1-dione (53.5 mg, 25.2%). LCMS:(ES, m/z): [M+H]:482.15. ¹H NMR (300 MHz, Methanol-d₄) δ 7.91 (d, J=1.9 Hz, 1H), 7.28 (d, J=1.9 Hz, 1H), 7.00 (dd, J=8.4, 5.7 Hz, 1H), 6.73 (dd, J=12.1, 8.4 Hz, 1H), 5.53 (dd, J=11.7, 1.8 Hz, 1H), 5.35 (d, J=14.8 Hz, 1H), 5.05 (d, J=14.7 Hz, 1H), 3.93 (dtd, J=12.8, 7.6, 6.1 Hz, 1H), 2.90 (s, 3H), 2.39 (s, 3H), 2.23 (s, 3H), 1.46 (dd, J=7.0, 1.1 Hz, 3H).

### Example 6: 5-((1S,2R)-1-(6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

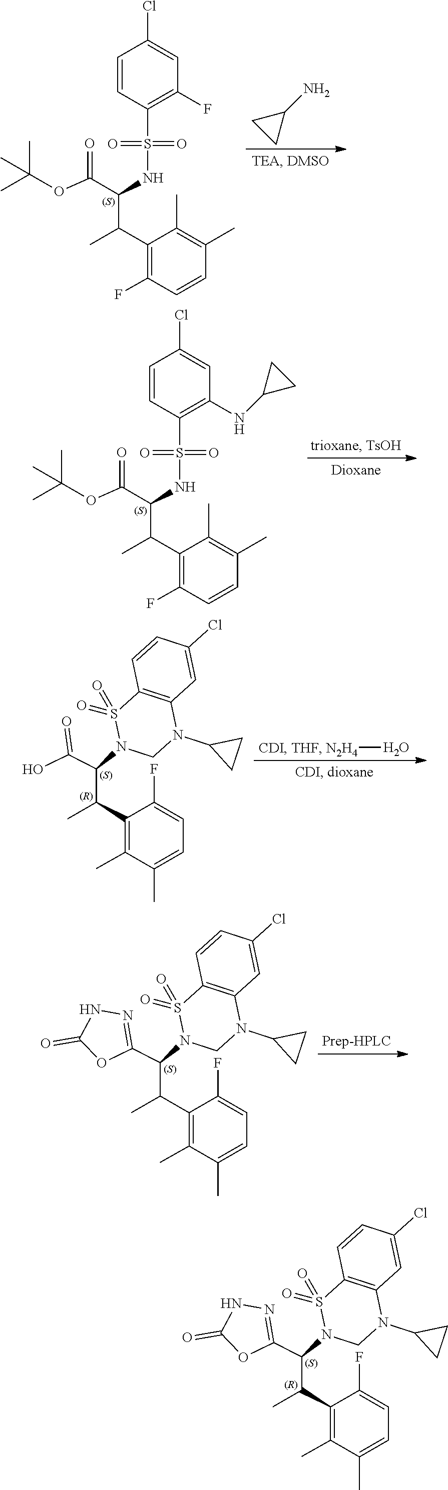

### Step 1: Synthesis of tert-butyl (2S)-2-[4-chloro-2-(cyclopropylamino) benzenesulfonamido]-3-(6-fluoro-2,3-dimethylphenyl) butanoate

To a stirred mixture of tert-butyl (2S)-2-(4-chloro-2-fluorobenzenesulfonamido)-3-(6-fluoro-2,3-dimethylphenyl) butanoate (See Ex 4, step 3) (10 mg, 0.021 mmol, 1 equiv) and DMSO (14 mL) were added TEA (3.20 mL, 23 mmol, 14 equiv) and aminocyclopropane (1.32 g, 23 mmol, 14 equiv) in portions at room temperature under air atmosphere. The resulting mixture was stirred for overnight at 80° C. under air atmosphere. The resulting mixture was quenched with water and extracted with EtOAc (3×20 mL). The combined organic layers were washed with brine (2×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in tert-butyl (2S)-2-[4-chloro-2-(cyclopropylamino) benzenesulfonamido]-3-(6-fluoro-2,3-dimethylphenyl) butanoate (713 mg, 84.8%).

### Step 2: Synthesis of (2S)-2-(6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid

Into a 20 mL vial were added tert-butyl (2S)-2-[4-chloro-2-(cyclopropylamino) benzenesulfonamido]-3-(6-fluoro-2,3-dimethylphenyl) butanoate (180 mg, 0.35 mmol, 1 equiv), dioxane (3.6 mL), 1,3,5-trioxane (317 mg, 3.52 mmol, 10 equiv) and TsOH (61 mg, 0.35 mmol, 1 equiv) at room temperature. The resulting mixture was stirred overnight at 100° C. under air atmosphere. The resulting mixture was concentrated under reduced pressure. The residue was purified by reverse flash chromatography with the following conditions: column, C18 silica gel; mobile phase, MeCN in Water (0.1% FA), 70% to 72% gradient in 10 min; detector, UV 254 nm. This resulted in (2S)-2-(6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid (92 mg, 55.9%).

### Step 3: Synthesis of 5-((1S)-1-(6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added (2S)-2-(6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid (80 mg, 0.17 mmol, 1 equiv), CDI (128 mg, 0.79 mmol, 4.6 equiv) and THF (1.6 mL) at room temperature. To the above mixture was added hydrazine hydrate (25.7 mg, 0.51 mmol, 3 equiv) dropwise at 0° C. The resulting mixture was stirred for additional 1 h at 0° C. The resulting mixture was quenched with water and extracted with EtOAc (2×30 mL). The combined organic layers were washed with brine (1×30 mL), dried over anhydrous Na₂SO₄. After filtration, the filtrate was concentrated under reduced pressure. A mixture of the above product and CDI (128 mg, 0.79 mmol, 4.6 equiv) in 1,4-dioxane was stirred overnight at room temperature under air atmosphere. The residue was purified by reverse flash chromatography with the following conditions: column, C18 silica gel; mobile phase, MeCN in Water (0.1% FA), 10% to 50% gradient in 10 min; detector, UV 254 nm. This resulted in 5-((1S)-1-(6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (80 mg, 92.1%).

The product was further purified by reverse flash chromatography with the following conditions: Column: XBridge Shield RP18 OBD Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 254 nm; RT1 (min): 7. This resulted in 6-chloro-4-cyclopropyl-2-[(1S,2R)-2-(6-fluoro-2,3-dimethylphenyl)-1-(5-oxo-4H-1,3,4-oxadiazol-2-yl)propyl]-3H-1lambda6,2,4-benzothiadiazine-1,1-dione (72.3 mg, 41.11%). LCMS:(ES, m/z):[M−H]⁺=505.05. ¹H NMR (300 MHz, Methanol-d₄) δ7.57 (d, J=8.4 Hz, 1H), 7.21 (d, J=1.9 Hz, 1H), 6.99 (dd, J=8.4, 5.7 Hz, 1H), 6.85 (dd, J=8.4, 1.9 Hz, 1H), 6.72 (dd, J=12.1, 8.4 Hz, 1H), 5.43 (dd, J=11.8, 1.8 Hz, 1H), 5.26 (d, J=14.4 Hz, 1H), 5.10 (d, J=14.4 Hz, 1H), 4.00-3.83 (m, 1H), 2.44-2.36 (m, 4H), 2.22 (s, 3H), 1.45 (d, J=6.9 Hz, 3H), 1.08-0.85 (m, 2H), 0.85-0.64 (m, 2H).

### Example 7: 5-((1S,2R)-1-(6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

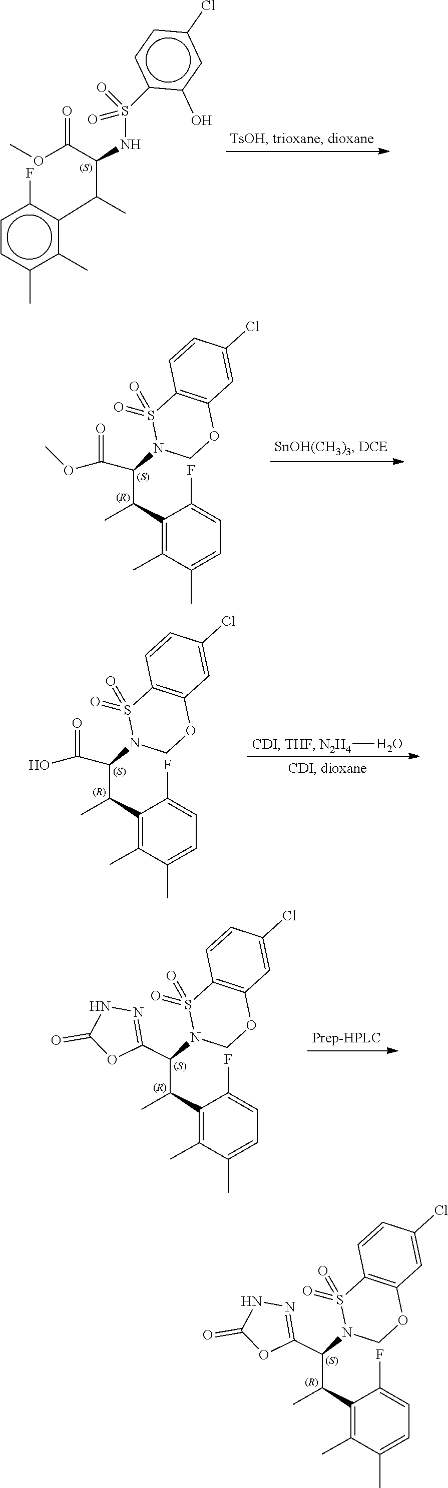

### Step 1: Synthesis of methyl (2S)-2-(6-chloro-1,1-dioxidobenzo[e][1,4,3]oxathiazin-2 (3H)-yl)-3-(6-fluoro-2,3-dimethylphenyl)butanoate

Into a 40 mL vial were added methyl (2S)-2-(4-chloro-2-hydroxybenzenesulfonamido)-3-(6-fluoro-2,3-dimethylphenyl) butanoate (see ex 2) (860 mg, 2 mmol, 1 equiv), dioxane (34.4 mL), trioxane (2.70 g, 30.0 mmol, 15 equiv) and TsOH (344 mg, 2 mmol, 1 equiv) at room temperature. The resulting mixture was stirred overnight at 100° C. under nitrogen atmosphere. The resulting mixture was quenched with water and extracted with EtOAc (2×50 mL). The combined organic layers were washed with brine (1×50 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in methyl (2S)-2-(6-chloro-1,1-dioxidobenzo[e][1,4,3]oxathiazin-2 (3H)-yl)-3-(6-fluoro-2,3-dimethylphenyl)butanoate (470 mg, 53.2%).

### Step 2: Synthesis of (2S)-2-(6-chloro-1,1-dioxidobenzo[e][1,4,3]oxathiazin-2 (3H)-yl)-3-(6-fluoro-2,3-dimethylphenyl)butanoic acid

Into a 100 mL round-bottom flask were added methyl (2S)-2-(6-chloro-1,1-dioxidobenzo[e][1,4,3]oxathiazin-2 (3H)-yl)-3-(6-fluoro-2,3-dimethylphenyl)butanoate (470 mg, 1 mmol, 1 equiv), DCE (18 mL) and trimethyltin hydroxide (1923 mg, 10 mmol, 10 equiv) at room temperature. The resulting mixture was stirred overnight at 60° C. under air atmosphere. The resulting mixture was quenched with water and extracted with EtOAc (2×50 mL). The combined organic layers were washed with brine (1×50 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 70% to 75% gradient in 10 min; detector, UV 254 nm. This resulted in (2S)-2-(6-chloro-1,1-dioxidobenzo[e][1,4,3]oxathiazin-2 (3H)-yl)-3-(6-fluoro-2,3-dimethylphenyl)butanoic acid (230 mg, 50.5%).

### Step 3: Synthesis of 5-((1S,2R)-1-(6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

A solution of (2S)-2-(6-chloro-1,1-dioxidobenzo[e][1,4,3]oxathiazin-2 (3H)-yl)-3-(6-fluoro-2,3-dimethylphenyl)butanoic acid (212 mg, 0.5 mmol, 1 equiv) and CDI (400 mg, 2.5 mmol, 5 equiv) in THF was stirred for 20 min at room temperature under air atmosphere. To the above mixture was added hydrazine hydrate (124 mg, 2.5 mmol, 5 equiv) dropwise at 0° C. The resulting mixture was stirred for additional 1 h at 0° C. The resulting mixture was quenched with water and extracted with EtOAc (2×30 mL). The combined organic layers were washed with brine (1×30 mL), dried over anhydrous Na₂SO₄. After filtration, the filtrate was concentrated under reduced pressure. Into a 50 mL round-bottom flask were added above crude product, dioxane (5 mL) and CDI (400 mg, 2.5 mmol, 5 equiv) at room temperature. The resulting mixture was stirred for 0.5h at room temperature under air atmosphere. The resulting mixture was extracted with EtOAc (2×100 mL). The combined organic layers were washed with brine (1×50 mL), dried over anhydrous Na₂SO₄. After filtration, the filtrate was concentrated under reduced pressure.

The residue was purified by reverse flash chromatography with the following conditions: Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 52% B in 8 min, 52% B; Wave Length: 254 nm; RT1 (min): 7. This resulted in 5-((1S,2R)-1-(6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (35.9 mg, 16.2%). LCMS:(ES, m/z): [M−H]⁺=485.15. ¹H NMR (300 MHz, Methanol-d₄) δ 7.63 (d, J=8.6 Hz, 1H), 7.07 (dd, J=8.6, 2.0 Hz, 1H), 6.99-6.84 (m, 2H), 6.64 (dd, J=12.1, 8.4 Hz, 1H), 5.82 (q, J=13.0 Hz, 2H), 5.50 (dd, J=11.8, 1.7 Hz, 1H), 3.83 (ddt, J=13.9, 7.0, 5.5 Hz, 1H), 2.29 (s, 3H), 2.13 (s, 3H), 1.35 (dd, J=6.9, 1.1 Hz, 3H).

### Example 8: 5-((1S,2R)-1-(6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

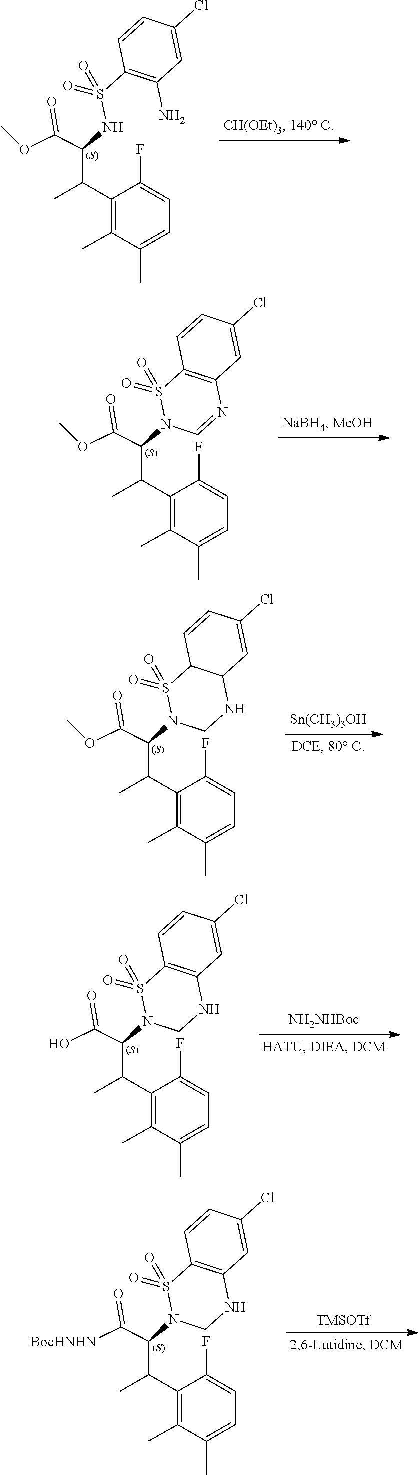

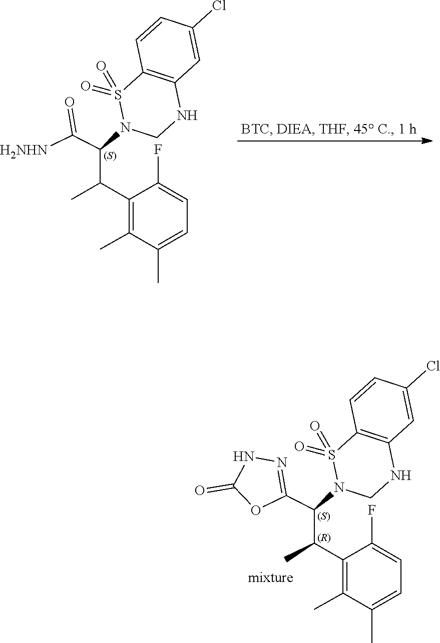

### Step 1: Synthesis of methyl (2S)-2-(6-chloro-1,1-dioxido-2H-benzo[e][1,2,4]thiadiazin-2-yl)-3-(6-fluoro-2,3-dimethylphenyl)butanoate

To a stirred solution of methyl (2S)-2-(2-amino-4-chlorobenzenesulfonamido)-3-(6-fluoro-2,3-dimethylphenyl) butanoate (12.7 g, 29.6 mmol, 1 equiv) in (diethoxymethoxy)ethane (250 mL). The resulting mixture was stirred for overnight at 145° C. The resulting mixture was concentrated under vacuum. The crude product was used in the next step directly without further purification.

### Step 2: Synthesis of methyl (2S)-2-(6-chloro-1,1-dioxido-3,4-dihydro-2H-benzo[e][1,2,4]thiadiazin-2-yl)-3-(6-fluoro-2,3-dimethylphenyl)butanoate

To a stirred solution of methyl (2S)-2-(6-chloro-1,1-dioxido-2H-benzo[e][1,2,4]thiadiazin-2-yl)-3-(6-fluoro-2,3-dimethylphenyl)butanoate (6.9 g, 2.27 mmol, 1 equiv) in DCE (15 mL) were added trimethyltin hydroxide (14.2 g, 78.2 mmol, 5 equiv) dropwise at room temperature. The resulting mixture was stirred for 2 days at 65° C. The resulting mixture was filtered; the filter cake was washed with DCM (2×50 mL). The filtrate was concentrated under reduced pressure. The residue was purified by reverse flash chromatography with the following conditions: column, C18 silica gel; mobile phase, MeCN in Water (0.1% FA), 10% to 100% gradient in 10 min; detector, UV 254 nm. This resulted in methyl (2S)-2-(6-chloro-1,1-dioxido-3,4-dihydro-2H-benzo[e][1,2,4] thiadiazin-2-yl)-3-(6-fluoro-2,3-dimethylphenyl)butanoate (1.8 g, 26.9%).

### Step 3: Synthesis of (2S)-2-(6-chloro-1,1-dioxido-3,4-dihydro-2H-benzo[e][1,2,4]thiadiazin-2-yl)-3-(6-fluoro-2,3-dimethylphenyl)butanoic acid

To a stirred solution of methyl (2S)-2-(6-chloro-1,1-dioxido-3,4-dihydro-2H-benzo[e][1,2,4] thiadiazin-2-yl)-3-(6-fluoro-2,3-dimethylphenyl)butanoate (6.9 g, 2.27 mmol, 1 equiv) in DCE (15 mL) were added trimethyltin hydroxide (14.2 g, 78.2 mmol, 5 equiv) dropwise at room temperature. The resulting mixture was stirred for 2 days at 65° C. The resulting mixture was filtered; the filter cake was washed with DCM (2×50 mL). The filtrate was concentrated under reduced pressure. The residue was purified by reverse flash chromatography with the following conditions: column, C18 silica gel; mobile phase, MeCN in Water (0.1% FA), 10% to 100% gradient in 10 min; detector, UV 254 nm. This resulted in (2S)-2-(6-chloro-1,1-dioxido-3,4-dihydro-2H-benzo[e][1,2,4]thiadiazin-2-yl)-3-(6-fluoro-2,3-dimethylphenyl)butanoic acid (1.8 g, 26.9%).

### Step 4: Synthesis of tert-butyl 2-((2S)-2-(6-chloro-1,1-dioxido-3,4-dihydro-2H-benzo[e][1,2,4]thiadiazin-2-yl)-3-(6-fluoro-2,3-dimethylphenyl)butanoyl)hydrazine-1-carboxylate

Into a 50 mL round-bottom flask were added 2-(6-chloro-1,1-dioxo-3,4-dihydro-1lambda6,2,4-benzothiadiazin-2-yl)-3-(6-fluoro-2,3-dimethylphenyl) butanoic acid (1.8 g, 4.2 mmol, 1 equiv) in DCM (20 mL) at room temperature. To the above mixture was added HATU (1.92 g, 5 mmol, 1.2 equiv) tert-butoxycarbohydrazide (0.84 g, 6.3 mmol, 1.5 equiv) DIEA (2.20 mL, 12.7 mmol, 3 equiv) at room temperature. The resulting mixture was stirred for additional 30 min at room temperature. The residue was purified by silica gel column chromatography, eluted with PE/EtOAc (8:1) to afford tert-butyl 2-((2S)-2-(6-chloro-1,1-dioxido-3,4-dihydro-2H-benzo[e][1,2,4]thiadiazin-2-yl)-3-(6-fluoro-2,3-dimethylphenyl)butanoyl)hydrazine-1-carboxylate (2 g, 87.2%).

### Step 5: Synthesis of (2S)-2-(6-chloro-1,1-dioxido-3,4-dihydro-2H-benzo[e][1,2,4]thiadiazin-2-yl)-3-(6-fluoro-2,3-dimethylphenyl)butanehydrazide

To a stirred solution tert-butyl 2-((2S)-2-(6-chloro-1,1-dioxido-3,4-dihydro-2H-benzo[e][1,2,4]thiadiazin-2-yl)-3-(6-fluoro-2,3-dimethylphenyl)butanoyl)hydrazine-1-carboxylate (1.99 g, 3.7 mmol, 1 equiv) in DCM (30 mL) were added trimethylsilyl triflate (10.7 mL, 58.8 mmol, 16 equiv) and 2,6-Lutidine (8.6 mL, 73.6 mmol, 20 equiv) dropwise at 0° C. The resulting mixture was stirred 1 h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in (2S)-2-(6-chloro-1,1-dioxido-3,4-dihydro-2H-benzo[e][1,2,4]thiadiazin-2-yl)-3-(6-fluoro-2,3-dimethylphenyl)butanehydrazide (1.4 g, 86.33%).

### Step 6: Synthesis of 5-((1S,2R)-1-(6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred mixture of (2S)-2-(6-chloro-1,1-dioxido-3,4-dihydro-2H-benzo[e][1,2,4]thiadiazin-2-yl)-3-(6-fluoro-2,3-dimethylphenyl)butanehydrazide (2.8 g, 6.35 mmol, 1 equiv) in THF (37 mL) were added DIEA (2.77 mL, 15.88 mmol, 2.5 equiv) and Triphosgene (15.1 mg, 0.05 mmol, 0.5 equiv) in portions at room temperature under air atmosphere. The resulting mixture was stirred for 40 min at 45° C. The resulting mixture was concentrated under vacuum. The residue was purified by reverse flash chromatography with the following conditions: column, CIS silica gel; mobile phase, MeCN in Water (10 mmol/L NH₄HCO₃), 10% to 100% gradient in 15 min; detector, UV 254 nm. This resulted in 6-chloro-2-[(1S,2R)-2-(6-fluoro-2,3-dimethylphenyl)-1-(5-oxo-4H-1,3,4-oxadiazol-2-yl)propyl]-3,4-dihydro-1lambda6,2,4-benzothiadiazine-1,1-dione (2.0171 g, 68.03%). LCMS:(ES, m/z): (M−H) 465.05. ¹H NMR (300 MHz, Methanol-d₄) δ 7.51-7.48 (d, J=8.5 Hz, 1H), 7.01-6.96 (dd, J=8.4, 5.8 Hz, 1H), 6.74-6.63 (m, 3H), 5.43-5.39 (dt, J=11.7, 1.2 Hz, 1H), 5.25-5.21 (d, J=14.6 Hz, 1H), 5.09-5.04 (d, J=14.6 Hz, 1H), 3.90-3.79 (m, 1H), 2.36 (s, 3H), 2.21 (s, 3H), 1.51-1.46 (dd, J=6.9, 1.1 Hz, 3H).

### Example 9; 5-((1S,2R)-1-(6-chloro-4-(methyl-d3)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

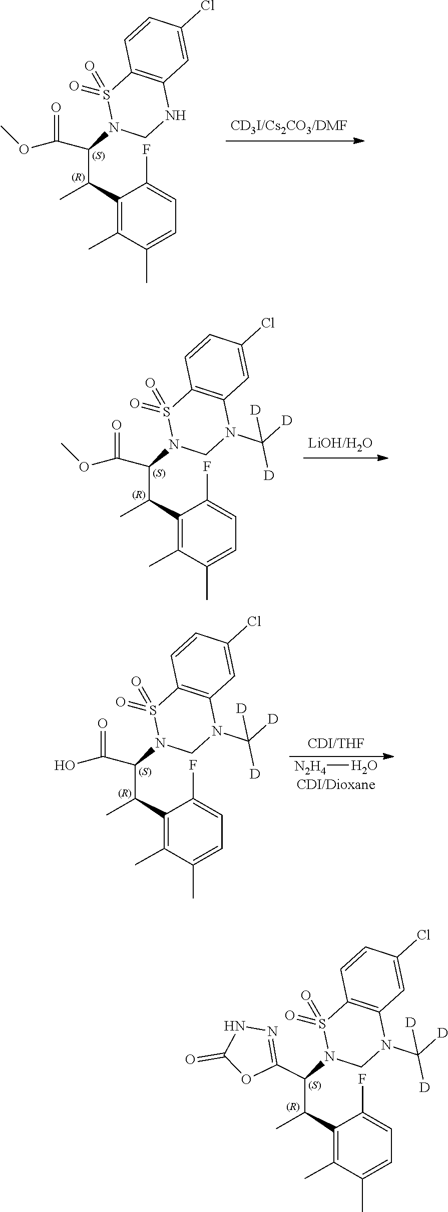

### Step 1: Synthesis of tert-butyl (2S)-2-((4-chloro-2-((methyl-d3)amino)phenyl)sulfonamido)-3-(6-fluoro-2,3-dimethylphenyl)butanoate

To a stirred solution of tert-butyl (2S)-2-(4-chloro-2-fluorobenzenesulfonamido)-3-(6-fluoro-2,3-dimethylphenyl) butanoate (see Ex 4, step 3) (1 g, 2.1 mmol, 1 equiv) in DMSO (10 mL) were added TEA (2.9 mL, 21 mmol, 10 equiv) and methyl-d₃-amine hydrochloride (744 mg, 10.6 mmol, 5 equiv) in portions at room temperature. The resulting mixture was stirred for 2 days at 80° C. The residue was purified by reverse flash chromatography with the following conditions: column, C18 silica gel; mobile phase, MeCN in Water (0.1% FA), 10% to 100% gradient in 10 min; detector, UV 254 nm. This resulted in tert-butyl(2S)-2-{4-chloro-2-[(D3) methylamino]benzenesulfonamido}-3-(6-fluoro-2,3-dimethylphenyl) butanoate (1 g, 97.1%).

### Step 2: Synthesis of (2S)-2-(6-chloro-4-(methyl-d3)-1,1-dioxido-3,4-dihydro-2H-benzo[e][1,2,4]thiadiazin-2-yl)-3-(6-fluoro-2,3-dimethylphenyl)butanoic acid

To a stirred solution of tert-butyl (2S)-2-{4-chloro-2-[(D3) methylamino]benzenesulfonamido}-3-(6-fluoro-2,3-dimethylphenyl) butanoate (500 mg, 1.03 mmol, 1 equiv) in dioxane (20 mL) were added TsOH (176 mg, 1.03 mmol, 1 equiv) and 1,3,5-trioxane (923 mg, 10.3 mmol, 10 equiv) in portions at room temperature. The resulting mixture was stirred overnight at 110° C. The residue was purified by reverse flash chromatography with the following conditions: column, C18 silica gel; mobile phase, MeCN in Water (10 mmol/L NH₄HCO₃), 10% to 70% gradient in 20 min; detector, UV 254 nm. This resulted in 2S)-2-(6-chloro-4-(methyl-d3)-1,1-dioxido-3,4-dihydro-2H-benzo[e][1,2,4]thiadiazin-2-yl)-3-(6-fluoro-2,3-dimethylphenyl)butanoic acid (240 mg, 52.8%).

### Step 2: Synthesis of 5-((1S)-1-(6-chloro-4-(methyl-d3)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added 2S)-2-(6-chloro-4-(methyl-d3)-1,1-dioxido-3,4-dihydro-2H-benzo[e][1,2,4]thiadiazin-2-yl)-3-(6-fluoro-2,3-dimethylphenyl)butanoic acid (230 mg, 0.52 mmol, 1 equiv) in THF (3 mL). To the above mixture was added CDI (126 mg, 0.78 mmol, 1.5 equiv) in portions at room temperature. The resulting mixture was stirred for additional 30 min at room temperature.

To the above mixture was added N₂H₄·H₂O (75.5 μL, 1.55 mmol, 3 equiv) dropwise at 0° C. The resulting mixture was stirred for additional 30 min at 0° C. The reaction was quenched with water (5 ml) at 0° C. The resulting mixture was extracted with EtOAc (2×5 mL). The combined organic layers were washed with brine (1×10 mL), dried over anhydrous Na₂SO₄. After filtration, the filtrate was concentrated under reduced pressure. The residue was loaded into a 50 mL round-bottom flask dioxane (3 mL) was added. To the above mixture was added CDI (210 mg, 1.3 mmol, 2.5 equiv) in portions at room temperature. The resulting mixture was stirred for additional 30 min at room temperature. The resulting mixture was concentrated under vacuum. The crude product (200 mg) was purified by Prep-HPLC with the following conditions (Column: XBridge Prep OBD CIS Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 22% B to 52% B in 9 min, 52% B; Wave Length: 254 nm; RT1 (min): 7.8 to afford 6-chloro-2-[(1S,2R)-2-(6-fluoro-2,3-dimethylphenyl)-1-(5-oxo-4H-1,3,4-oxadiazol-2-yl)propyl]-4-(2H3)methyl-3H-1lambda6,2,4-benzothiadiazine-1,1-dione (36.7 mg, 17.4%). LC: (ES, m/z): [M+H+17]⁺=501.25. ¹H NMR (400 MHz, Methanol-d₄) δ 7.58-7.56 (d, J=8.4 Hz, 1H), 7.01-6.97 (dd, J=8.4, 5.7 Hz, 1H), 6.80-6.77 (dd, J=8.5, 1.9 Hz, 1H), 6.74-6.68 (m, 2H), 5.45-5.42 (d, J=11.7 Hz, 1H), 5.38-5.32 (d, J=14.6 Hz, 1H), 5.06-4.97 (d, J=14.5 Hz, 1H), 3.93-3.85 (dq, J=13.2, 6.8 Hz, 1H), 2.38 (s, 3H), 2.22 (s, 3H), 1.45 (d, J=6.9 Hz, 3H).

### Example 10: 5-((1S,2R)-1-(6-chloro-1,1-dioxido-4-propyl-3,4-dihydro-2H-benzo[e][1,2,4]thiadiazin-2-yl)-2-(6-fluoro-2,3-dimethylphenyl)propyl)-1,3,4-oxadiazol-2 (3H)-one

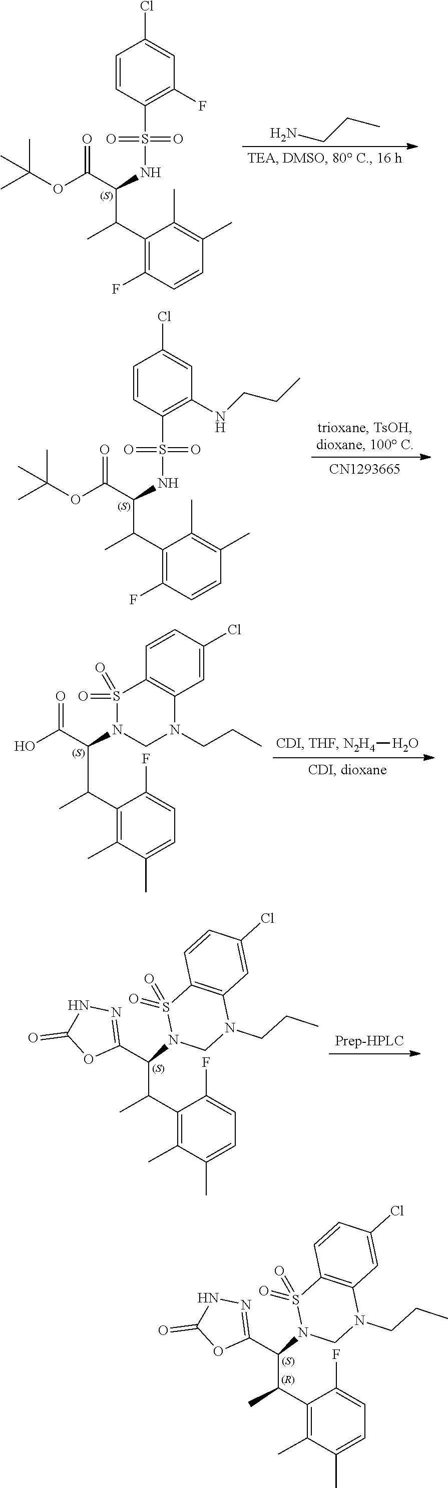

10020415-((1S,2R)-1-(6-chloro-1,1-dioxido-4-propyl-3,4-dihydro-2H-benzo[e][1,2,4]thiadiazin-2-yl)-2-(6-fluoro-2,3-dimethylphenyl)propyl)-1,3,4-oxadiazol-2 (3H)-one was prepared according to example 33 starting (2S)-2-[4-chloro-2-(propylamino)benzenesulfonamido]-3-(6-fluoro-2,3-dimethylphenyl)butanoate (285 mg, 0.019 mmol, 1 equiv) and propylamine (260 μL, 3.17 mmol, 5 equiv). The final product (180 mg) was purified by Prep-HPLC with the following conditions (Column: XBridge Prep OBD C18 Column, 30*150 mm; Mobile Phase A: Water(10 mmol/L NH₄HCO₃+0.1% NH₃·H₂O), Mobile Phase B: ACN; Flow rate: 60 mL/min; Gradient: 32% B to 62% B in 9 min, 62% B; Wave Length: 254 nm; RT1 (min): 7) to afford 6-chloro-2-[(1S,2R)-2-(6-fluoro-2,3-dimethylphenyl)-1-(5-oxo-4H-1,3,4-oxadiazol-2-yl)propyl]-4-propyl-3H-1lambda6,2,4-benzothiadiazine-1,1-dione (60.4 mg, 33.25%). LC-MS: (ES, m/z): [M−H]⁺=507.10. ¹H NMR (300 MHz, Methanol-d₄) δ 7.57-7.55 (d, J=8.4 Hz, 1H), 7.01-6.96 (dd, J=8.3, 5.8 Hz, 1H), 6.77-6.67 (m, 3H), 5.44-5.36 (dd, J=13.2, 10.4 Hz, 2H), 5.11-5.06 (d, J=14.6 Hz, 1H), 3.93-3.87 (dq, J=13.3, 7.0 Hz, 1H), 3.46-3.36 (dt, J=14.9, 7.4 Hz, 1H), 3.14-3.04 (dt, J=15.5, 8H).

### Example 11: 5-((1S,2R)-1-(6-chloro-3,4-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

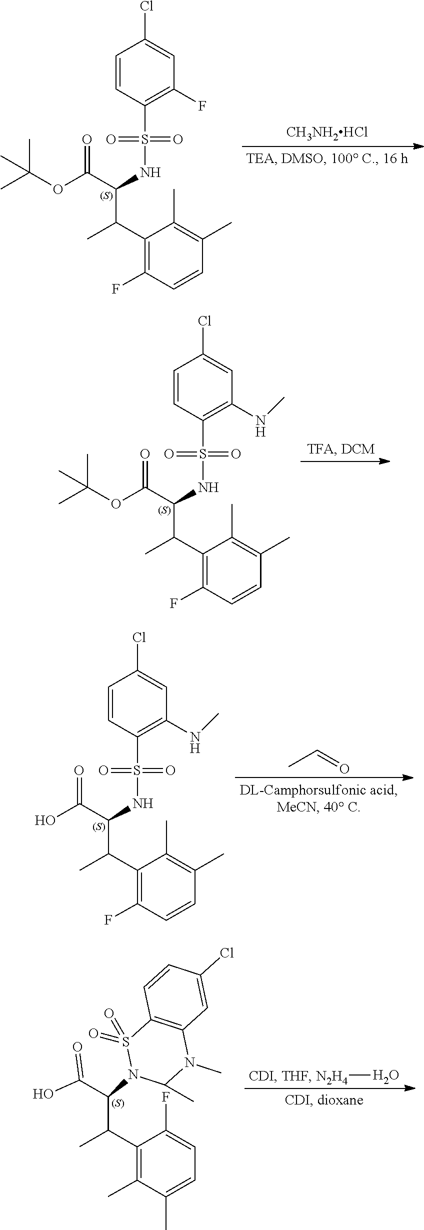

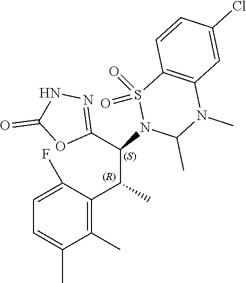

### Step 1: Synthesis of tert-butyl (2S)-2-[4-chloro-2-(methylamino) benzenesulfonamido]-3-(6-fluoro-2,3-dimethylphenyl) butanoate

Into a 40 mL vial were added tert-butyl (2S)-2-(4-chloro-2-fluorobenzenesulfonamido)-3-(6-fluoro-2,3-dimethylphenyl) butanoate (1.5 g, 3.17 mmol, 1 equiv), methylamine (2M in THF) (15.8 mL, 31.7 mmol, 10 equiv) and TEA (4.4 mL, 31.7 mmol, 10 equiv) in DMSO. The resulting mixture was stirred overnight at 65° C. After evaporation, the residue was purified by reverse flash chromatography with the following conditions: column, C18 silica gel; mobile phase, MeCN in Water (0.1% FA), 10% to 50% gradient in 10 min; detector, UV 254 nm. This resulted in tert-butyl (2S)-2-[4-chloro-2-(methylamino) benzenesulfonamido]-3-(6-fluoro-2,3-dimethylphenyl) butanoate (1.2 g, 78.2%).

### Step 2: Synthesis of 2S)-2-[4-chloro-2-(methylamino) benzenesulfonamido]-3-(6-fluoro-2,3-dimethylphenyl) butanoic acid

Into a 40 mL vial were added tert-butyl (2S)-2-[4-chloro-2-(methylamino) benzenesulfonamido]-3-(6-fluoro-2,3-dimethylphenyl)butanoate (1 g, 2.0 mmol, 1 equiv), DCM (5 mL) and TFA (3 mL) at room temperature. The resulting mixture was stirred for overnight at room temperature. The resulting mixture was concentrated under vacuum. The residue was purified by reverse flash chromatography with the following conditions: column, C18 silica gel; mobile phase, MeCN in Water (10 mmol/L NH₄HCO₃), 10% to 50% gradient in 10 min; detector, UV 254 nm. This resulted in (2S)-2-[4-chloro-2-(methylamino) benzenesulfonamido]-3-(6-fluoro-2,3-dimethylphenyl) butanoic acid (800 mg, 90.5%).

### Step 3: Synthesis of (2S)-2-(6-chloro-3,4-dimethyl-1,1-dioxido-3,4-dihydro-2H-benzo[e][1,2,4]thiadiazin-2-yl)-3-(6-fluoro-2,3-dimethylphenyl)butanoic acid

Into a 20 mL vial were added (2S)-2-[4-chloro-2-(methylamino) benzenesulfonamido]-3-(6-fluoro-2,3-dimethylphenyl) butanoic acid (1 g, 2.33 mmol, 1 equiv), MeCN (10 mL), acetaldehyde (3081 mg, 70 mmol, 30 equiv) and DL-Camphor sulfonic acid (541 mg, 2.33 mmol, 1 equiv) at room temperature. The resulting mixture was stirred for 3 h at 45° C. The resulting mixture was concentrated under vacuum. The residue was purified by reverse flash chromatography with the following conditions: column, C18 silica gel; mobile phase, MeCN in Water (0.1% FA), 10% to 50% gradient in 10 min; detector, UV 254 nm. This resulted in (2S)-2-(6-chloro-3,4-dimethyl-1,1-dioxido-3,4-dihydro-2H-benzo[e][1,2,4]thiadiazin-2-yl)-3-(6-fluoro-2,3-dimethylphenyl)butanoic acid (200 mg, 18.9%).

### Step 4: Synthesis of 5-((1S,2R)-1-(6-chloro-3,4-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 8 mL vial were added (2S)-2-(6-chloro-3,4-dimethyl-1,1-dioxido-3,4-dihydro-2H-benzo[e][1,2,4]thiadiazin-2-yl)-3-(6-fluoro-2,3-dimethylphenyl)butanoic acid (100 mg, 0.22 mmol, 1 equiv), TIF (0.5 mL) and CDI (71.3 mg, 0.44 mmol, 2 equiv) at room temperature. The resulting mixture was stirred for 30 min at room temperature. To the above mixture was added NH₂NH₂·H₂O (33.0 mg, 0.66 mmol, 3 equiv) dropwise at 0° C. The resulting mixture was stirred for additional 30 min at 0° C. The resulting mixture was extracted with EtOAc. The combined organic layers were washed with brine, dried over anhydrous Na₂SO₄. After filtration, the filtrate was concentrated under reduced pressure. To the above mixture was added dioxane (0.5 mL) and CDI (92.7 mg, 0.57 mmol, 2.6 equiv) dropwise at room temperature. The resulting mixture was stirred for additional 30 min at room temperature. The resulting mixture was extracted with EtOAc. The combined organic layers were washed with brine, dried over anhydrous Na₂SO₄. After filtration, the filtrate was concentrated under reduced pressure. The crude product (was purified by Chiral-Prep-HPLC with the following conditions: Column, XBridge Shield RP18 OBD Column, 30*150 mm, 5 μm; mobile phase, Water (10 mmol/L NH₄HCO₃+0.1% NH₃·H₂O) and MeOH-Preparative (20% MeOH-Preparative up to 50% in 8 min); Detector, uv 254 nm. This resulted in 5-((1 S,2R)-1-(6-chloro-3,4-dimethyl-1,1-dioxido-3,4-dihydro-2H-benzo[e][1,2,4] thiadiazin-2-yl)-2-(6-fluoro-2,3-dimethylphenyl)propyl)-1,3,4-oxadiazol-2 (3H)-one (23.3 mg, 19.8%).

LCMS:(ES, m/z): [M+H]: 495.10. ¹H NMR (300 MHz, Methanol-d₄) δ 7.60 (d, J=8.4 Hz, 1H) 7.02 (dd, J=8.4, 5.8 Hz, 1H), 6.83 (dd, J=8.4, 1.9 Hz, 1H), 6.82-6.74 (m, 1H), 6.74-6.67 (m, 1H), 5.66 (dd, J=11.8, 2.1 Hz, 1H), 5.27 (q, J=6.7 Hz, 1H), 3.91 (tt, J=13.1, 6.2 Hz, 1H), 2.87 (s, 3H), 2.41 (s, 3H), 2.25 (s, 3H), 1.78 (d, J=6.8 Hz, 3H), 1.50 (dd, J=6.9, 1.2 Hz, 3H).

### Example 12: 5-((1S,2R)-1-(6-chloro-4,7-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

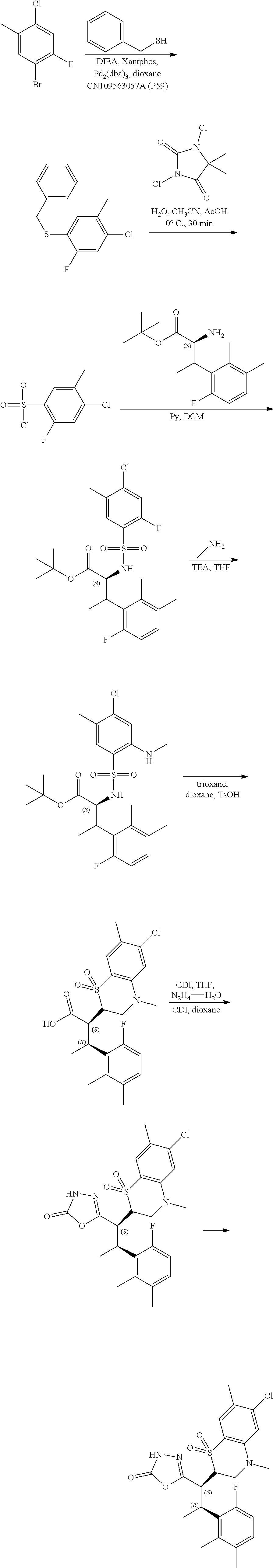

### Step 1: Synthesis of 1-(benzylsulfanyl)-4-chloro-2-fluoro-5-methylbenzene

To a stirred solution of 1-bromo-4-chloro-2-fluoro-5-methylbenzene (5 g, 22.3 mmol, equiv) and DIEA (11.7 mL, 67.1 mmol, 3 equiv) in dioxane was added Xantphos (2.59 g, 4.48 mmol, 0.2 equiv) Pd₂(dba)₃ (2.0 g, 2.24 mmol, 0.1 equiv) in portions at room temperature. The resulting mixture was stirred overnight at 100° C. under nitrogen atmosphere. The reaction was quenched with water at room temperature. The resulting mixture was extracted with EtOAc (2×20 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc (10:1) to afford 1-(benzylsulfanyl)-4-chloro-2-fluoro-5-methylbenzene (5.8 g, 97.2%).

### Step 2: Synthesis of 4-chloro-2-fluoro-5-methylbenzenesulfonyl chloride

To a stirred solution of 1-(benzylsulfanyl)-4-chloro-2-fluoro-5-methylbenzene (2 g, 7.5 mmol, 1 equiv) and H₂O (1.22 mL, 67.5 mmol, 9 equiv) in MeCN and AcOH (2.44 mL, 37.5 mmol, 5 equiv) was added 1,3-dichloro-5,5-dimethylimidazolidine-2,4-dione (2.95 g, 15 mmol, 2 equiv) in portions at 0° C. The resulting mixture was stirred for 30 min at 0° C. The reaction was quenched with Water/Ice at room temperature. The resulting mixture was extracted with EtOAc (2×10 mL). The combined organic layers were washed with brine (2×20 mL), dried over anhydrous Na₂SO₄. The resulting mixture was concentrated under reduced pressure. This resulted in 4-chloro-2-fluoro-5-methylbenzenesulfonyl chloride (3.6 g, crude).

### Step 3: Synthesis of tert-butyl (2S)-2-(4-chloro-2-fluoro-5-methylbenzenesulfonamido)-3-(6-fluoro-2,3-dimethylphenyl) butanoate

To a stirred solution of tert-butyl (2S)-2-amino-3-(6-fluoro-2,3-dimethylphenyl) butanoate (2.5 g, 8.9 mmol, 0.9 equiv) and Pyridine (10 mL) was added 4-chloro-2-fluoro-5-methylbenzenesulfonyl chloride (3.6 g, 14.8 mmol, 1.5 equiv) DCM (40 mL) dropwise at 0° C. The resulting mixture was stirred for overnight at room temperature. The resulting mixture was extracted with CH₂Cl₂ (3×50 mL). The combined organic layers were washed with brine (2×200 mL), dried over anhydrous MgSO₄. After filtration, the filtrate was concentrated under reduced pressure. The residue was purified by silica gel column chromatography, eluted with PE/EtOAc (10:1) to afford tert-butyl (2S)-2-(4-chloro-2-fluoro-5-methylbenzenesulfonamido)-3-(6-fluoro-2,3-dimethylphenyl) butanoate (2.7 g, 56%).

### Step 4: Synthesis of tert-butyl (2S)-2-[4-chloro-5-methyl-2-(methylamino) benzenesulfonamido]-3-(6-fluoro-2,3-dimethylphenyl) butanoate

Into a 20 mL vial were added tert-butyl (2S)-2-(4-chloro-2-fluoro-5-methylbenzenesulfonamido)-3-(6-fluoro-2,3-dimethylphenyl) butanoate (500 mg, 1 mmol, 1 equiv), methylamine (5.12 mL, 5.13 mmol, 5 equiv) and TEA (1.42 mL, 10 mmol, 10 equiv) in THF at room temperature. The resulting mixture was stirred overnight at 80° C. under air atmosphere. The residue was purified by reverse flash chromatography with the following conditions: column, C18 silica gel; mobile phase, MeCN in Water (10 mmol/L NH₄HCO₃), 100% gradient in 10 min; detector, UV 254 nm. This resulted in tert-butyl (2S)-2-[4-chloro-5-methyl-2-(methylamino) benzenesulfonamido]-3-(6-fluoro-2,3-dimethylphenyl) butanoate (400 mg, 78.2%).

### Step 5: Synthesis of (2S,3R)-2-(6-chloro-4,7-dimethyl-1,1-dioxido-3,4-dihydro-2H-benzo[e][1,2,4]thiadiazin-2-yl)-3-(6-fluoro-2,3-dimethylphenyl)butanoic acid

Into a 20 mL vial were added tert-butyl (2S)-2-[4-chloro-5-methyl-2-(methylamino) benzenesulfonamido]-3-(6-fluoro-2,3-dimethylphenyl) butanoate (350 mg, 0.7 mmol, 1 equiv), dioxane (7 mL), trioxane (632 mg, 7.0 mmol, 10 equiv) and TsOH (121 mg, 0.70 mmol, 1 equiv) at room temperature. The resulting mixture was stirred overnight at 100° C. under air atmosphere. The resulting mixture was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in (2S)-2-(6-chloro-4,7-dimethyl-1,1-dioxido-3,4-dihydro-2H-benzo[e][1,2,4]thiadiazin-2-yl)-3-(6-fluoro-2,3-dimethylphenyl)butanoic acid (228 mg, 71.5%).

### Step 6: Synthesis of 5-((1S,2R)-1-(6-chloro-4,7-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 10 mL vial were added (2S)-2-(6-chloro-4,7-dimethyl-1,1-dioxo-3H-1lambda6,2,4-benzothiadiazin-2-yl)-3-(6-fluoro-2,3-dimethylphenyl) butanoic acid (208 mg, 0.46 mmol, 1 equiv), THF (0.4 mL) and CDI (341 mg, 2.1 mmol, 4.6 equiv) at room temperature. The resulting mixture was stirred for 20 min at room temperature under air atmosphere. To the above mixture was added NH₂NH₂·H₂O (111 μL, 2.29 mmol, 5 equiv) dropwise at 0° C. The resulting mixture was stirred for additional 30 min at 0° C. The resulting mixture was quenched with water and extracted with EtOAc (2×5 mL). The combined organic layers were washed with brine (1×5 mL), dried over anhydrous Na₂SO₄. After filtration, the filtrate was concentrated under reduced pressure. Into a 10 mL vial were added the above crude product, dioxane (4 mL) and CDI (341 mg, 2.1 mmol, 4.6 equiv) at room temperature. The resulting mixture was stirred for 0.5h at room temperature under air atmosphere. The resulting mixture was quenched with water and extracted with EtOAc (3×50 mL). The combined organic layers were washed with brine (1×100 mL), dried over anhydrous Na₂SO₄. After filtration, the filtrate was concentrated under reduced pressure. 1002151The crude was purified by reverse flash chromatography with the following conditions: Column: XBridge Shield RP18 OBD Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 26% B to 56% B in 9 min, 56% B; Wave Length: 254 nm; RT1 (min): 7. This resulted in 6-chloro-2-[(1S,2R)-2-(6-fluoro-2,3-dimethylphenyl)-1-(5-oxo-4H-1,3,4-oxadiazol-2-yl)propyl]-4,7-dimethyl-3H-1lambda6,2,4-benzothiadiazine-1,1-dione (64.5 mg, 31.77%). LCMS:(ES, m/z):[M−H]⁺=493.10. ¹H NMR (300 MHz, Methanol-d₄) δ7.43-7.37 (m, 1H), 6.87 (dd, J=8.4, 5.7 Hz, 1H), 6.68-6.54 (m, 2H), 5.40-5.30 (m, 1H), 5.20 (d, J=14.4 Hz, 1H), 4.89 (d, J=14.4 Hz, 1H), 3.78 (dqd, J=11.8, 6.9, 1.6 Hz, 1H), 2.74 (s, 3H), 2.28 (s, 3H), 2.14 (d, J=15.4 Hz, 6H), 1.35 (dd, J=6.9, 1.1 Hz, 3H).

### Example 13: 5-((1S,2R)-1-(7-chloro-5-methyl-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

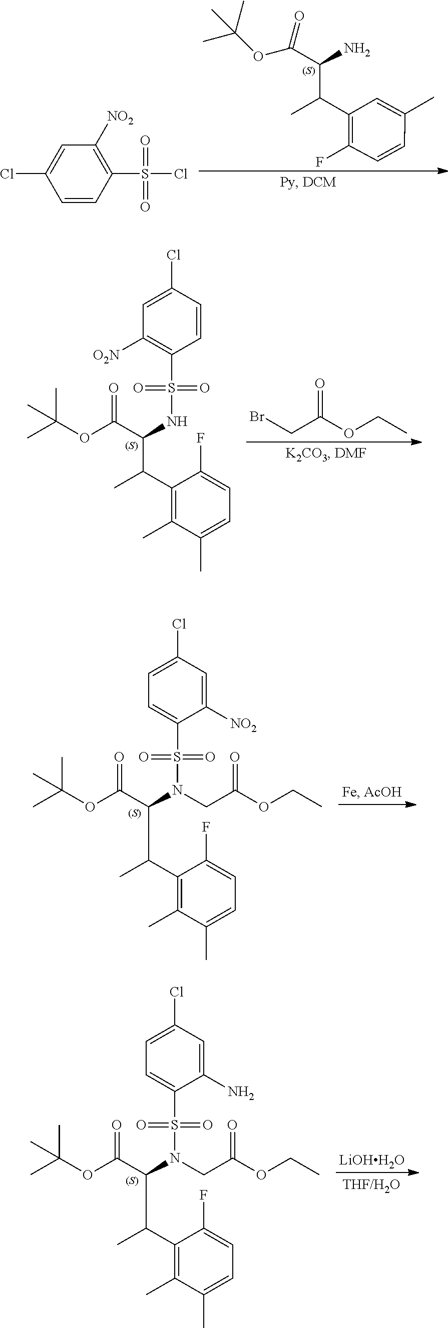

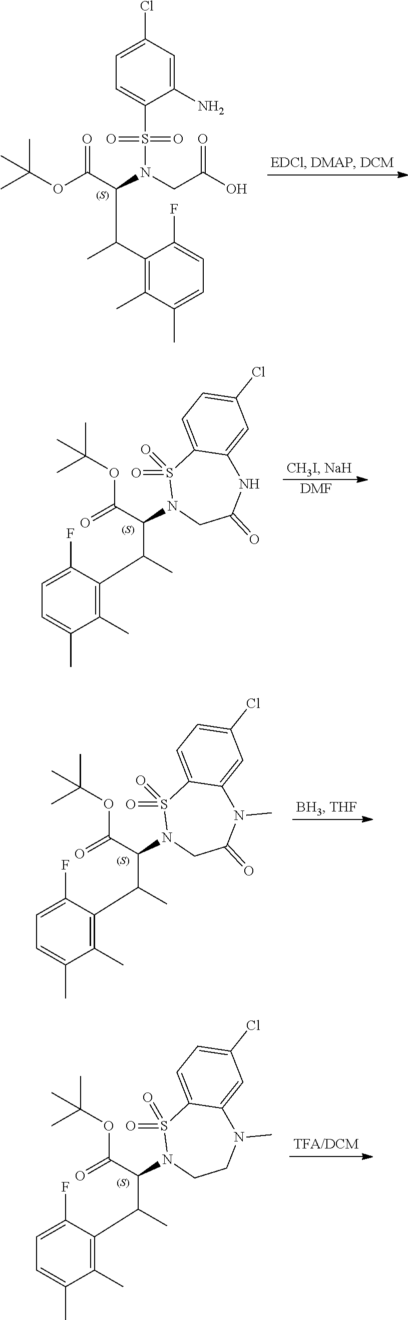

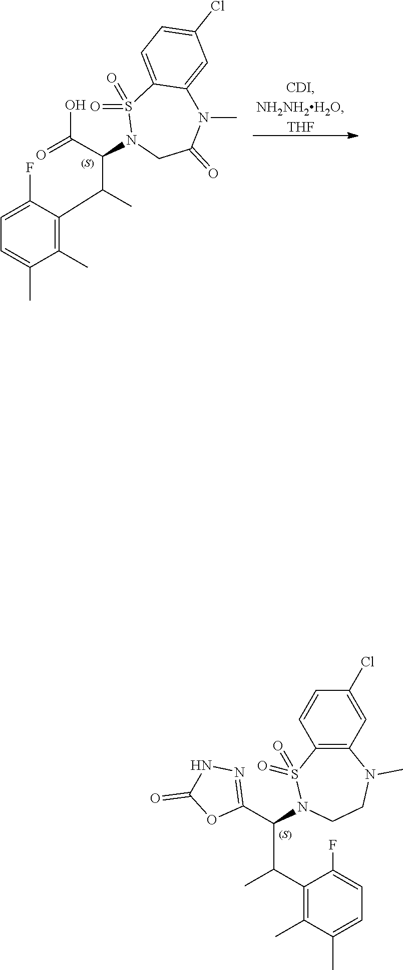

### Step 1: Synthesis of tert-butyl (2S)-2-(4-chloro-2-nitrobenzenesulfonamido)-3-(6-fluoro-2,3-dimethylphenyl) butanoate

To a stirred solution of tert-butyl (2S)-2-amino-3-(6-fluoro-2,3-dimethylphenyl) butanoate (5.10 g, 18.1 mmol, 0.8 equiv), pyridine (18.3 mL, 226 mmol, 10 equiv) in DCM was added 4-chloro-2-nitrobenzenesulfonyl chloride (prepared as described in example 16)(5.8 g, 22.7 mmol, 1 equiv) in DCM dropwise at 0° C. The resulting mixture was stirred for overnight at room temperature. The resulting mixture was concentrated under vacuum. The residue was purified by silica gel column chromatography, eluted with PE/EtOAc (10:1) to afford tert-butyl (2S)-2-(4-chloro-2-nitrobenzenesulfonamido)-3-(6-fluoro-2,3-dimethylphenyl) butanoate (6.2 g, 54.6%).

### Step 2: Synthesis of (2S)-2-[N-(2-ethoxy-2-oxoethyl)4-chloro-2-nitrobenzenesulfonamido]-3-(6-fluoro-2,3-dimethylphenyl) butanoate

To a stirred solution of tert-butyl (2S)-2-(4-chloro-2-nitrobenzenesulfonamido)-3-(6-fluoro-2,3-dimethylphenyl) butanoate (1.8 g, 3.59 mmol, 1 equiv) and K₂CO₃ (1 g, 7.19 mmol, 2 equiv) in DMF was added ethyl bromoacetate (398 μL, 3.59 mmol, 1 equiv) in portions at room temperature. The resulting mixture was stirred for 60 min at 60° C. The mixture was allowed to cool down to room temperature. The reaction was quenched with Water at room temperature. The resulting mixture was extracted with EtOAc (3×25 mL). The combined organic layers were washed with brine (1×100 mL), dried over anhydrous Na₂SO₄.

After filtration, the filtrate was concentrated under reduced pressure. The residue was purified by silica gel column chromatography, eluted with PE/EtOAc (12:1) to afford tert-butyl (2S)-2-[N-(2-ethoxy-2-oxoethyl)4-chloro-2-nitrobenzenesulfonamido]-3-(6-fluoro-2,3-dimethylphenyl) butanoate (1.33 g, 63%).

### Step 3: Synthesis of tert-butyl (2S)-2-[N-(2-ethoxy-2-oxoethyl)2-amino-4-chlorobenzenesulfonamido]-3-(6-fluoro-2,3-dimethylphenyl) butanoate

To a stirred solution of methyl (2S)-2-(4-chloro-2-nitrobenzenesulfonamido)-3-(6-fluoro-2,3-dimethylphenyl) butanoate (1.33 g, 2.1 mmol, 1 equiv) in AcOH (15 mL) was added Fe (1.24 g, 22.1 mmol, 10 equiv) in portions at room temperature. The resulting mixture was stirred 15 min at 70° C. The mixture was allowed to cool down to room temperature. The resulting mixture was filtered, the filter cake was washed with ethyl acetate (3×30 mL). The filtrate was concentrated under reduced pressure. The residue was purified by reverse flash chromatography with the following conditions: column, C18 silica gel; mobile phase, MeCN in Water (0.1% FA), 10% to 100% gradient in 20 min; detector, UV 254 nm. This resulted in tert-butyl (2S)-2-[N-(2-ethoxy-2-oxoethyl)2-amino-4-chlorobenzenesulfonamido]-3-(6-fluoro-2,3-dimethylphenyl) butanoate (987 mg, 80%).

### Step 4: Synthesis of N-((2-amino-4-chlorophenyl)sulfonyl)-N-((2S)-1-(tert-butoxy)-3-(6-fluoro-2,3-dimethylphenyl)-1-oxobutan-2-yl)glycine

To a stirred solution of tert-butyl (2S)-2-[N-(2-ethoxy-2-oxoethyl)2-amino-4-chlorobenzenesulfonamido]-3-(6-fluoro-2,3-dimethylphenyl) butanoate (950 mg, 1.71 mmol, 1 equiv) in THF and H₂O (9.50 mL) was added lithium hydrate (358 mg, 8.53 mmol, 5 equiv) in portions at room temperature. The resulting mixture was stirred overnight at 65° C. The mixture was acidified to pH 5 with HCl (2M). The resulting mixture was extracted with EtOAc (3×25 mL). The combined organic layers were washed with brine (1×50 mL), dried over anhydrous Na₂SO₄. After filtration, the filtrate was concentrated under reduced pressure. The crude product/resulting mixture was used in the next step directly without further purification.

### Step 5: Synthesis of tert-butyl (2S)-2-(7-chloro-1,1-dioxido-4-oxo-4,5-dihydrobenzo[f][1,2,5] thiadiazepin-2 (3H)-yl)-3-(6-fluoro-2,3-dimethylphenyl)butanoate

To a stirred solution of {N-[(2S)-1-(tert-butoxy)-3-(6-fluoro-2,3-dimethylphenyl)-1-oxobutan-2-yl]2-amino-4-chlorobenzenesulfonamido} acetic acid (800 mg, 1.5 mmol, 1 equiv) in DCM was added EDCI (319 mg, 1.67 mmol, 1.1 equiv) in portions at room temperature. To the above mixture was added DMAP (18.5 mg, 0.15 mmol, 0.1 equiv) at room temperature. The resulting mixture was stirred for additional 2h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in of tert-butyl (2S)-2-(7-chloro-1,1-dioxido-4-oxo-4,5-dihydrobenzo[f][1,2,5]thiadiazepin-2 (3H)-yl)-3-(6-fluoro-2,3-dimethylphenyl)butanoate (430 mg, 55.6%).

### Step 6: Synthesis of tert-butyl (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoate

To a solution of tert-butyl (2S)-2-(7-chloro-1,1-dioxido-4-oxo-4,5-dihydrobenzo[f][1,2,5] thiadiazepin-2 (3H)-yl)-3-(6-fluoro-2,3-dimethylphenyl)butanoate (200 mg, 0.39 mmol, 1 equiv) in DMF was added sodium hydride (60% in oil, 24 mg) at 0° C. The mixture was stirred for 15 min. CH₃I (48.7 μL, 0.78 mmol, 2 equiv) was added and the mixture was allowed to warm to RT and stirred for 1h. The reaction was quenched with water at room temperature. The resulting mixture was extracted with EtOAc (3×10 mL).

The combined organic layers were washed with brine (1×20 mL), dried over anhydrous Na₂SO₄. After filtration, the filtrate was concentrated under reduced pressure. The residue was purified by silica gel column chromatography, eluted with PE/EtOAc (3:1) to tert-butyl (2S)-2-(7-chloro-5-methyl-1,1-dioxido-4-oxo-4,5-dihydrobenzo[f][1,2,5] thiadiazepin-2 (3H)-yl)-3-(6-fluoro-2,3-dimethylphenyl)butanoate (180 mg, 87.60%).

### Step 7: Synthesis of tert-butyl (2S)-2-(7-chloro-5-methyl-1,1-dioxido-4,5-dihydrobenzo[f][1,2,5]thiadiazepin-2 (3H)-yl)-3-(6-fluoro-2,3-dimethylphenyl)butanoate

To a stirred solution of tert-butyl (2S)-2-(7-chloro-5-methyl-1,1-dioxido-4-oxo-4,5-dihydrobenzo[f][1,2,5] thiadiazepin-2 (3H)-yl)-3-(6-fluoro-2,3-dimethylphenyl)butanoate (300 mg, 0.57 mmol, 1 equiv) in THF (6 mL) was added BH₃-THF (3 mL, 3 mmol, 5.3 equiv) dropwise at room temperature. The resulting mixture was stirred for 2 h at 65° C. under nitrogen atmosphere. The reaction was quenched by the addition of MeOH (1 mL) at room temperature. The resulting mixture was stirred 1 h at 65° C. The resulting mixture was concentrated under vacuum. The crude product was used in the next step directly without further purification.

### Step 8: Synthesis of (2S)-2-(7-chloro-5-methyl-1,1-dioxido-4,5-dihydrobenzo[f][1,2,5]thiadiazepin-2 (3H)-yl)-3-(6-fluoro-2,3-dimethylphenyl)butanoic acid

To a stirred solution of tert-butyl (2S)-2-(7-chloro-5-methyl-1,1-dioxido-4,5-dihydrobenzo[f][1,2,5]thiadiazepin-2 (3H)-yl)-3-(6-fluoro-2,3-dimethylphenyl)butanoate (280 mg, 0.55 mmol, 1 equiv) in DCM (4 mL) was added TFA (4 mL) dropwise at room temperature. The resulting mixture was stirred overnight at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in (2S)-2-(7-chloro-5-methyl-1,1-dioxido-4,5-dihydrobenzo[f][1,2,5]thiadiazepin-2 (3H)-yl)-3-(6-fluoro-2,3-dimethylphenyl)butanoic acid.

### Step 9: Synthesis of 5-((1S,2R)-1-(7-chloro-5-methyl-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

To a stirred solution of 2S)-2-(7-chloro-5-methyl-1,1-dioxido-4,5-dihydrobenzo[f][1,2,5]thiadiazepin-2 (3H)-yl)-3-(6-fluoro-2,3-dimethylphenyl)butanoic acid (188 mg, 0.4 mmol, 1 equiv) in THF (3.8 mL) was added CDI (97.5 mg, 0.6 mmol, 1.5 equiv) in portions at room temperature. The resulting mixture was stirred for 30 min at room temperature. To the above mixture was added hydrazine hydrate (58.5 μL, 1.2 mmol, 3 equiv) dropwise at 0° C. The resulting mixture was stirred for additional 10 min at 0° C. The reaction was quenched with water/ice at 0° C. The resulting mixture was extracted with EtOAc (2×10 mL). The combined organic layers were washed with brine (1×10 mL), dried over anhydrous Na₂SO₄.

After filtration, the filtrate was concentrated under reduced pressure. The residue was dissolved in 1,4-dioxane (4 mL). To the above mixture was added CDI (106 mg, 1.204 mmol, 3.0 equiv) in portions at room temperature. The resulting mixture was stirred for additional 1 h at room temperature. The resulting mixture was concentrated under vacuum.

The crude product was purified by Chiral-Prep-HPLC with the following conditions:Column, XBridge Prep OBD C18 Column, 19*250 mm, 5 μm; mobile phase, Water(10 mmol/L NH₄HCO₃+0.1% NH₃·H₂O) and MeOH-Preparative (57% MeOH-Preparative up to 70% in 10 min); Detector, UV 254 nm. This resulted in 5-((1S,2R)-1-(7-chloro-5-methyl-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (80 mg, 40%). LCMS:(ES, m/z)(M−H)=493.1. ¹H NMR (300 MHz, Methanol-d₄) δ 7.73-7.70 (dd, J=8.5, 1.6 Hz, 1H), 6.98-6.95 (t, J=7.1 Hz, 1H), 6.90-6.87 (dd, J=8.5, 2.0 Hz, 1H), 6.81 (d, J=1.9 Hz, 1H), 6.74-6.67 (dd, J=12.1, 8.4 Hz, 1H), 5.53-5.49 (d, J=11.7 Hz, 1H), 4.17-4.08 (m, 1H), 3.92-3.75 (m, 2H), 3.58-3.54 (m, 1H), 3.35 (d, J=4.6 Hz, 2H), 2.88 (d, J=1.6 Hz, 3H), 2.35 (s, 3H), 2.21 (s, 3H), 1.44 (d, J=6.9 Hz, 3H).

### Example 14: 5-((1S,2R)-1-(7-chloro-5-methyl-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

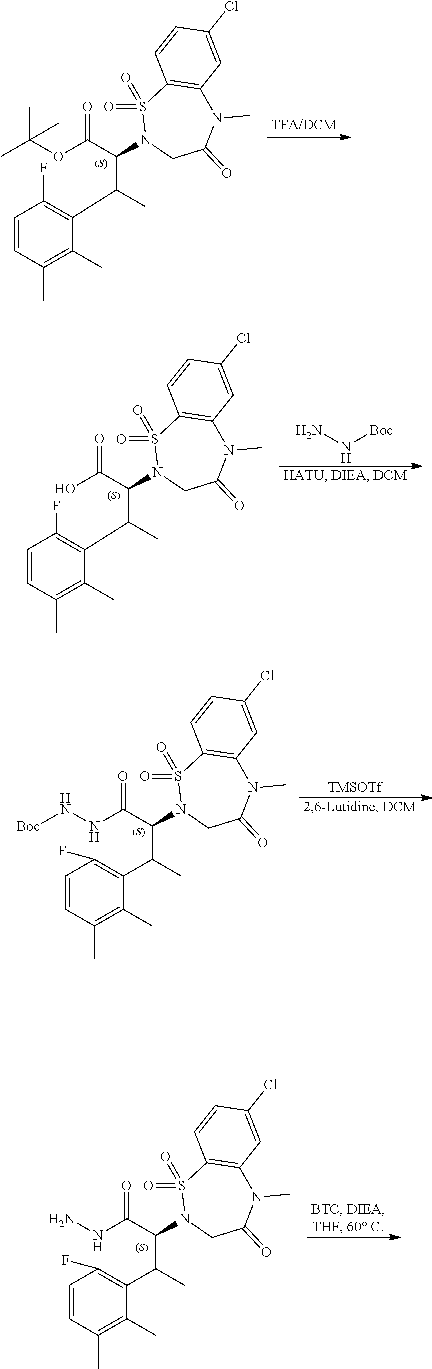

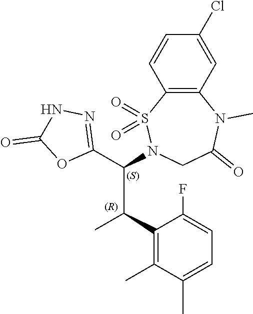

### Step 1: Synthesis of (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoic acid

To a stirred solution of (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoic acid (400 mg, 0.76 mmol, 1 equiv) in DCM (4 mL) was added TFA (2 mL) at room temperature. The resulting mixture was stirred for 30 min at room temperature. The resulting mixture was concentrated under vacuum. The residue was purified by reverse flash chromatography with the following conditions: column, C18 silica gel; mobile phase, MeCN in Water (0.1% FA), 10% to 60/gradient in 10 min; detector, UV 254 nm. This resulted in (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoic acid (290 mg, 81.18%).

### Step 2: Synthesis of tert-butyl 2-((2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate

To a stirred solution of (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoic acid (28 mg, 0.6 mmol, 1 equiv) and HATU (272 mg, 0.72 mmol, 1.2 equiv) in DCM (3 mL) was added DIEA (312 μL, 1.79 mmol, 3 equiv) at room temperature. To the above mixture was added tert-butoxycarbohydrazide (118 mg, 0.9 mmol, 1.5 equiv) at room temperature. The resulting mixture was stirred for additional 30 min at room temperature.

The residue was purified by silica gel column chromatography, eluted with PE/EtOAc (3:1) to afford tert-butyl 2-((2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate (305 mg, 87.60%).

### Step 3: Synthesis of (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanehydrazide

To a stirred solution of tert-butyl 2-((2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate (208 mg, 0.36 mmol, 1 equiv) in DCM (2 mL) were added 2,6-Lutidine (831 μL, 7.14 mmol, 20 equiv) and trimethylsilyl triflate (1033 μL, 5.71 mmol, 16 equiv) dropwise at 0° C. The resulting mixture was stirred 1 h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanehydrazide (96 mg, 55.7%).

### Step 4: Synthesis of 5-((1S,2R)-1-(7-chloro-5-methyl-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

To a stirred solution of (2S)-2-(7-chloro-5-methyl-1,1-dioxido-4-oxo-4,5-dihydrobenzo[f][1,2,5]thiadiazepin-2 (3H)-yl)-3-(6-fluoro-2,3-dimethylphenyl)butanehydrazide (96 mg, 0.2 mmol, 1 equiv) and DIEA (86.6 μL, 0.5 mmol, 2.5 equiv) in THF was added ditrichloromethyl carbonate (29.5 mg, 0.1 mmol, 0.5 equiv) in portions at room temperature. The resulting mixture was stirred 30 min at 60° C. The residue was purified by reverse flash chromatography with the following conditions: column, C18 silica gel; mobile phase, MeCN in Water (10 mmol/L NH₄HCO₃), 10% to 100% gradient in 10 min; detector, UV 254 nm. This resulted 5-((1S)-1-(7-chloro-5-methyl-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (53 mg, 52.4%).

The product was further purified by Prep-Achiral-SFC with the following conditions (Column: Torus 2-PIC Column, 4.6*100 mm, 5 μm; Mobile Phase B: ACN:MeOH=80:20 (1% 2M NH₃-MeOH); Flow rate: 4 mL/min; Gradient: isocratic 10% B; Wave Length: 220 nm) to afford 5-((1S,2R)-1-(7-chloro-5-methyl-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (29.1 mg, 57.97%/). LC-MS: (ES, m/z): [M+H]⁺=509.05. ¹H NMR (300 MHz, Methanol-d₄) δ 7.80-7.74 (m, 2H), 7.53-7.50 (d, J=8.6 Hz, 1H), 7.04-6.99 (t, J=7.4 Hz, 1H), 6.81-6.74 (m, 1H), 5.58-5.54 (d, J=11.8 Hz, 1H), 3.91-3.87 (d, J=11.3 Hz, 1H), 3.81-3.77 (dd, J=12.9, 6.6 Hz, 1H), 3.52-3.48 (d, J=11.8 Hz, 1H), 3.37 (s, 3H), 2.26 (s, 3H), 2.20 (s, 3H), 1.40 (d, J=6.9 Hz, 3H).

### Example 15: 5-((1S,2R)-1-(7-chloro-8-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

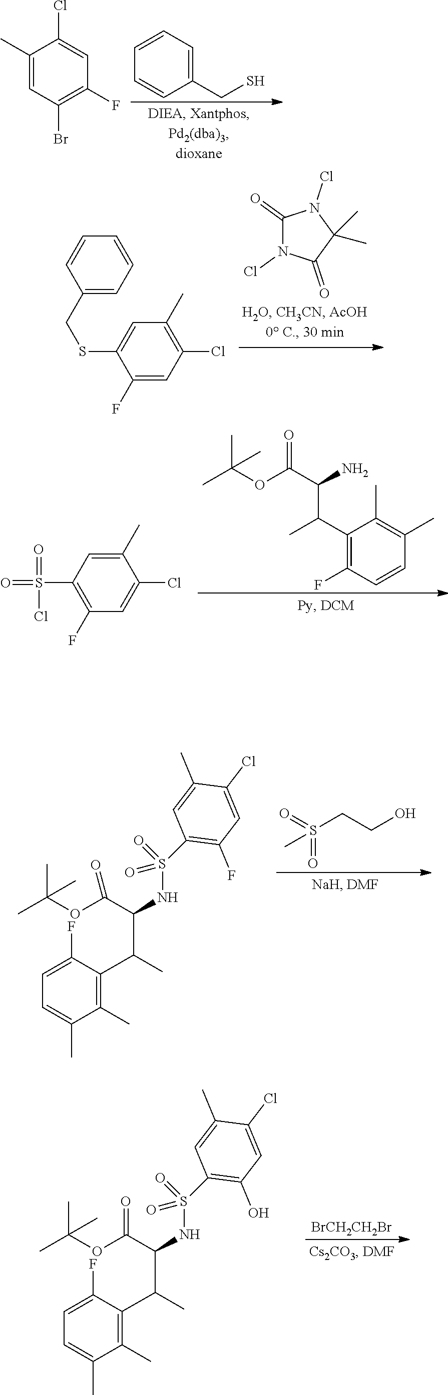

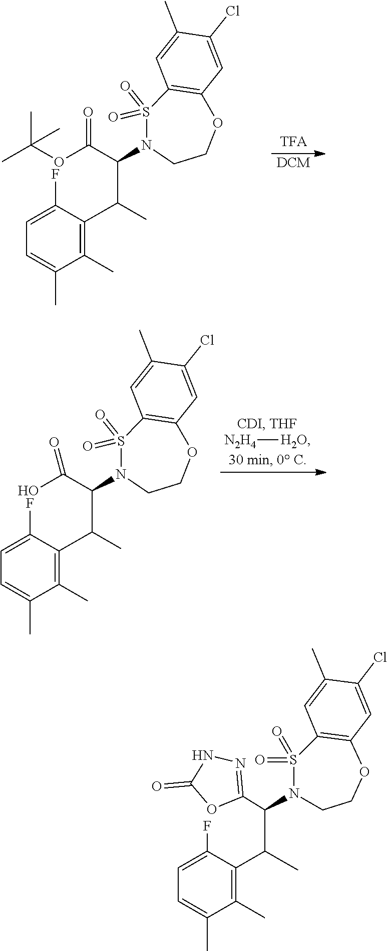

### Step 1: Synthesis of 1-(benzylsulfanyl)-4-chloro-2-fluoro-5-methylbenzene

To a stirred solution of 1-bromo-4-chloro-2-fluoro-5-methylbenzene (5 g, 22.4 mmol, 1 equiv) and DIEA (11.7 mL, 67.1 mmol, 3 equiv) in dioxane was added Xantphos (2.6 g, 4.5 mmol, 0.2 equiv) Pd₂(dba)₃ (2.1 g, 2.24 mmol, 0.1 equiv) in portions at room temperature. The resulting mixture was stirred overnight at 100° C. under nitrogen atmosphere. The reaction was quenched with water at room temperature. The resulting mixture was extracted with EtOAc (2×20 mL). The combined organic layers were washed with brine (2×30 mL), dried over anhydrous Na₂SO₄. After filtration, the filtrate was concentrated under reduced pressure. The residue was purified by silica gel column chromatography, eluted with PEI/EtOAc (10:1) to afford 1-(benzylsulfanyl)-4-chloro-2-fluoro-5-methylbenzene (5.8 g, 97.2%/).

### Step 2: Synthesis of 4-chloro-2-fluoro-5-methylbenzenesulfonyl chloride

To a stirred solution of 1-(benzylsulfanyl)-4-chloro-2-fluoro-5-methylbenzene (2 g, 7.5 mmol, 1 equiv) and H₂O (1.2 mL, 67.5 mmol, 9 equiv) in MeCN and AcOH (2.44 mL, 37.5 mmol, 5 equiv) was added 1,3-dichloro-5,5-dimethylimidazolidine-2,4-dione (3 g, 15 mmol, 2 equiv) in portions at 0° C. The resulting mixture was stirred 30 min at 0° C. The reaction was quenched with Water/Ice at room temperature. The resulting mixture was extracted with EtOAc (2×10 mL). The combined organic layers were washed with brine (2×20 mL), dried over anhydrous Na₂SO₄. The resulting mixture was concentrated under reduced pressure. This resulted in 4-chloro-2-fluoro-5-methylbenzenesulfonyl chloride (3.6 g, crude).

### Step 3: Synthesis of tert-butyl (2S)-2-(4-chloro-2-fluoro-5-methylbenzenesulfonamido)-3-(6-fluoro-2,3-dimethylphenyl) butanoate

To a stirred solution of tert-butyl (2S)-2-amino-3-(6-fluoro-2,3-dimethylphenyl) butanoate (2.5 g, 8.9 mmol, 1 equiv) and Pyridine (10 mL) was added 4-chloro-2-fluoro-5-methylbenzenesulfonyl chloride (3.6 g, 14.8 mmol, 1.5 equiv) DCM (40 mL) dropwise at 0° C. The resulting mixture was stirred overnight at room temperature. The resulting mixture was extracted with DCM (3×50 mL). The combined organic layers were washed with brine (2×200 mL), dried over anhydrous MgSO₄. After filtration, the filtrate was concentrated under reduced pressure. The residue was purified by silica gel column chromatography, eluted with PE/EtOAc (10:1) to afford tert-butyl (2S)-2-(4-chloro-2-fluoro-5-methylbenzenesulfonamido)-3-(6-fluoro-2,3-dimethylphenyl) butanoate (2.7 g, 56%).

### Step 4: Synthesis of afford (2S)-2-(4-chloro-2-hydroxy-5-methylbenzenesulfonamido)-3-(6-fluoro-2,3-dimethylphenyl) butanoate

To a stirred solution of tert-butyl (2S)-2-(4-chloro-2-fluoro-5-methylbenzenesulfonamido)-3-(6-fluoro-2,3-dimethylphenyl) butanoate (20 mg, 0.041 mmol, 1 equiv) and 2-methanesulfonylethanol (458 mg, 3.69 mmol, 3 equiv) in DMF was added NaH (246 mg, 6.15 mmol, 5 equiv, 60%) in portions at 0° C. The resulting mixture was stirred overnight at 80° C. The reaction was quenched with water at room temperature. The resulting mixture was extracted with EtOAc (2×20 mL). The combined organic layers were washed with brine (1×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (10 mmol/L NH₄HCO₃), 10% to 50% gradient in 10 min; detector, UV 254 nm. This resulted in tert-butyl (2S)-2-(4-chloro-2-hydroxy-5-methylbenzenesulfonamido)-3-(6-fluoro-2,3-dimethylphenyl) butanoate (100 mg, 16.7%).

### Step 5: Synthesis of tert-butyl ((2S)-2-(7-chloro-8-methyl-1,1-dioxido-3,4-dihydro-2H-benzo[b][1,4,5]oxathiazepin-2-yl-3-(6-fluoro-2,3-dimethylphenyl)butanoate

To a stirred solution of tert-butyl (2S)-2-(4-chloro-2-hydroxy-5-methylbenzenesulfonamido)-3-(6-fluoro-2,3-dimethylphenyl) butanoate (200 mg, 0.41 mmol, 1 equiv) and Cs₂CO₃ (402 mg, 1.24 mmol, 3 equiv) in DMF was added dibromoethane (77.3 mg, 0.41 mmol, 1 equiv) in portions at room temperature.

The resulting mixture was stirred for 1 h at 60° C. The reaction was quenched with water at room temperature. The residue was purified by reverse flash chromatography with the following conditions: column, C18 silica gel; mobile phase, MeCN in Water (0.1% FA), 10% to 50% gradient in 10 min; detector, UV 254 nm. This resulted in of tert-butyl ((2S)-2-(7-chloro-8-methyl-1,1-dioxido-3,4-dihydro-2H-benzo[b][1,4,5]oxathiazepin-2-yl)-3-(6-fluoro-2,3-dimethylphenyl)butanoate (130 mg, 61.7%).

### Step 6: Synthesis of (2S)-2-(7-chloro-8-methyl-1,1-dioxido-3,4-dihydro-2H-benzo[b][1,4,5]oxathiazepin-2-yl)-3-(6-fluoro-2,3-dimethylphenyl)butanoic acid

To a stirred solution of tert-butyl ((2S)-2-(7-chloro-8-methyl-1,1-dioxido-3,4-dihydro-2H-benzo[b][1,4,5]oxathiazepin-2-yl)-3-(6-fluoro-2,3-dimethylphenyl)butanoate (115 mg, 0.23 mmol, 1 equiv) and DCM (0.92 mL) was added trifluoroacetaldehyde (1 mL) at room temperature. The resulting mixture was stirred for 90 min at room temperature. The resulting mixture was concentrated under reduced pressure. The residue was purified by reverse flash chromatography with the following conditions: column, C18 silica gel; mobile phase, MeCN in Water (0.1% FA), 0% to 100% gradient in 20 min; detector, UV 220 nm. This resulted (2S)-2-(7-chloro-8-methyl-1,1-dioxido-3,4-dihydro-2H-benzo[b][1,4,5]oxathiazepin-2-yl)-3-(6-fluoro-2,3-dimethylphenyl)butanoic acid (80 mg, 78.12%).

### Step 7: Synthesis of 5-((1S,2R)-1-(7-chloro-8-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

To a stirred solution of (2S)-2-(7-chloro-8-methyl-1,1-dioxido-3,4-dihydro-2H-benzo[b][1,4,5]oxathiazepin-2-yl)-3-(6-fluoro-2,3-dimethylphenyl)butanoic acid (75 mg, 0.16 mmol, 1 equiv) in THF was added CDI (107 mg, 0.66 mmol, 4 equiv) in portions at room temperature. The resulting mixture was stirred 30 min at room temperature. To the above mixture was added NH₂NH₂·H₂O (24 μL, 0.49 mmol, 3 equiv) dropwise at 0° C. The resulting mixture was stirred 30 min at room temperature. The reaction was quenched with Water/Ice at room temperature. The resulting mixture was extracted with EtOAc (2×10 mL).

The combined organic layers were washed with brine (2×20 mL), dried over anhydrous Na₂SO₄. After filtration, the filtrate was concentrated under reduced pressure. To the above mixture was added CDI (107 mg, 0.66 mmol, 4 equiv) in dioxane (2 mL) dropwise at room temperature. The resulting mixture was stirred for additional 30 min at room temperature. The reaction was quenched with water at room temperature. The residue was purified by reverse flash chromatography with the following conditions: Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 254 nm; RT1 (min): 7. This resulted in 5-((1S,2R)-1-(7-chloro-8-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (34.6 mg, 41.7%). LC-MS: (ES, m/z): [M−H]=494.15. ¹H NMR (300 MHz, Methanol-d₄) δ 7.66-7.26 (s, 1H), 7.11 (d, J=1.7 Hz, 1H), 7.10-6.91 (t, J=7.2 Hz, 1H), 6.75-6.68 (dd, J=12.1, 8.4 Hz, 1H), 5.50-5.36 (d, J=11.6 Hz, 1H), 4.55-4.51 (dd, J=12.3, 5.6 Hz, 1H), 4.16-3.88 (m, 4H), 2.4-2.36 (dd, J=9.0, 1.7 Hz, 6H), 2.33-2.16 (s, 3H), 1.44-1.24 (d, J=7.0 Hz, 3H).

### Example 16: 5-((1S,2R)-1-(7-chloro-1,1-dioxido-3,4-dihydro-2H-pyrido[3,2-b][1,4,5]oxathiazepin-2-yl)-2-(6-fluoro-2,3-dimethylphenyl)propyl)-1,3,4-oxadiazol-2 (3H)-one

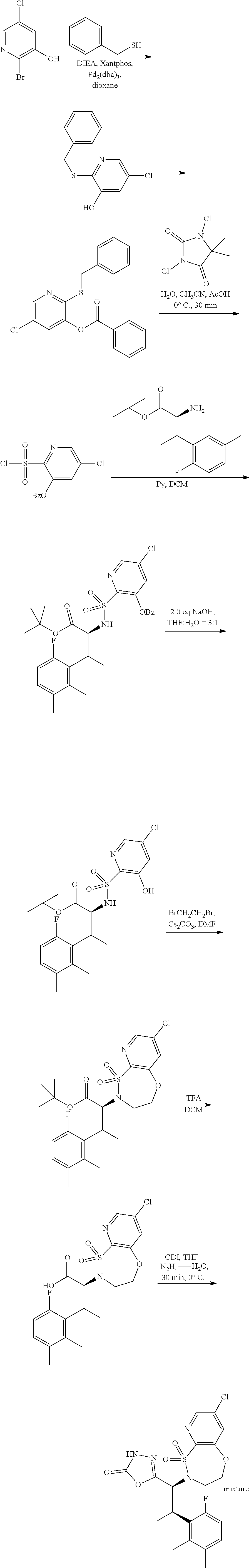

### Step 1: Synthesis of 2-(benzylsulfanyl)-5-chloropyridin-3-ol

Into a 250 mL round-bottom flask were added 2-bromo-5-chloropyridin-3-ol (6 g, 28.8 mmol, 1 equiv), Dioxane (120 mL), DIEA (15 mL, 86.4 mmol, 3 equiv), Xantphos (3.33 g, 5.8 mmol, 0.2 equiv), benzylmercaptan (3.4 mL, 28.8 mmol, 1 equiv) and Pd₂(dba)₃ (2.6 g, 2.9 mmol, 0.1 equiv) at 100° C. The resulting mixture was stirred overnight at 100° C. under nitrogen atmosphere. The resulting mixture was filtered, the filter cake was washed with ethyl acetate (3×10 mL). The filtrate was concentrated under reduced pressure. The residue was purified by silica gel column chromatography, eluted with PE/EtOAc (10:1) to afford 2-(benzylsulfanyl)-5-chloropyridin-3-ol (7 g, 96.6%).

### Step 2: Synthesis of 2-(benzylthio)-5-chloropyridin-3-yl benzoate

Into a 20 mL vial were added Benzoic acid (97 mg, 0.79 mmol, 2 equiv), DCM (2 mL), SOCl₂ (37.5 L, 0.52 mmol, 1.3 equiv) and DMF (1 drop) at room temperature. The resulting mixture was stirred for 1 h at room temperature to obtain intermediate A. Into another 8 mL vial were added 2-(benzylsulfanyl)-5-chloropyridin-3-ol (100 mg, 0.4 mmol, 1 equiv), DCM (2 mL) and Pyridine (35.4 μL, 0.44 mmol, 1.1 equiv) at 0° C. To the mixture was added intermediate A dropwise at 0° C. The resulting mixture was stirred for 30 min at 0° C. The residue was purified by reverse flash chromatography with the following conditions: column, C18 silica gel; mobile phase, MeCN in Water (0.1% FA), 0% to 100% gradient in 10 min; detector, UV 254 nm. This resulted in 2-(benzylthio)-5-chloropyridin-3-yl benzoate (89 mg, 63%).

### Step 3: Synthesis of 5-chloro-2-(chlorosulfonyl) pyridin-3-yl benzoate

Into a 20 mL vial were added 2-(benzylthio)-5-chloropyridin-3-yl benzoate (50 mg, 0.1 mmol, 1 equiv), CH: CN (5 ml), AcOH (14 μL, 0.25 mmol, 4.4 equiv) and H₂O (10 μL, 0.560 mmol, 10 equiv). To the mixture was added 1,3-dichloro-5,5-dimethylimidazolidine-2,4-dione (22.2 mg, 0.11 mmol, 2 equiv) at 0° C. The resulting mixture was quenched with water and extracted with EtOAc (3×10 mL). The combined organic layers were washed with brine (3×10 mL), dried over anhydrous Na₂SO₄. After filtration, the filtrate was concentrated under reduced pressure. This resulted in 5-chloro-2-(chlorosulfonyl)pyridin-3-yl benzoate (13 mg, 27%) as a light-yellow oil.

### Step 4: Synthesis of 2-(N-((2S)-1-(tert-butoxy)-3-(6-fluoro-2,3-dimethylphenyl)-1-oxobutan-2-yl)sulfamoyl-5-chloropyridin-3-yl benzoate

Into a 20 mL vial were added tert-butyl (2S)-2-amino-3-(6-fluoro-2,3-dimethylphenyl) butanoate (300 mg, 1.07 mmol, 17 equiv), Pyridine (3 mL). To the mixture was added 5-chloro-2-(chlorosulfonyl)pyridin-3-yl benzoate (531 mg, 1.6 mmol, 1.5 equiv) at 0° C. The resulting mixture was stirred for 1 h at room temperature. The residue was purified by silica gel column chromatography, eluted with PE/EtOAc (5:1) to afford 2-(N-((2S)-1-(tert-butoxy)-3-(6-fluoro-2,3-dimethylphenyl)-1-oxobutan-2-yl) sulfamoyl)-5-chloropyridin-3-yl benzoate (228 mg, 37%).

### Step 5: Synthesis of tert-butyl (2S)-2-((5-chloro-3-hydroxypyridine)-2-sulfonamido)-3-(6-fluoro-2,3-dimethylphenyl) butanoate

Into a 100 mL round-bottom flask were added 2-(N-((2S)-1-(tert-butoxy)-3-(6-fluoro-2,3-dimethylphenyl)-1-oxobutan-2-yl) sulfamoyl)-5-chloropyridin-3-yl benzoate (4.5 g, 7.8 mmol, 1 equiv), THF (38 mL), H₂O (12.5 mL) and NaOH (624 mg, 16 mmol, 2 equiv) at room temperature. The resulting mixture was stirred for 1 h at 50° C. The mixture was acidified to pH 6 with HCl (aq.). The resulting mixture was extracted with EtOAc (3×10 mL). The combined organic layers were washed with brine (3×10 mL), dried over anhydrous Na₂SO₄. After filtration, the filtrate was concentrated under reduced pressure.

The residue was purified by reverse flash chromatography with the following conditions: column, C18 silica gel; mobile phase, MeCN in Water (0.1% FA), 0/to 100% gradient in 20 min; detector, UV 254 nm. This resulted in Synthesis of tert-butyl (2S)-2-((5-chloro-3-hydroxypyridine)-2-sulfonamido)-3-(6-fluoro-2,3-dimethylphenyl) butanoate (2.5 g, 67.8%).

### Step 6: Synthesis of tert-butyl (2S)-2-(7-chloro-1,1-dioxido-3,4-dihydro-2H-pyrido[3,2-b][1,4,5]oxathiazepin-2-yl)-3-(6-fluoro-2,3-dimethylphenyl)butanoate

Into a 100 mL round-bottom flask were added tert-butyl (2S)-2-((5-chloro-3-hydroxypyridine)-2-sulfonamido)-3-(6-fluoro-2,3-dimethylphenyl) butanoate (2.5 g, 5.29 mmol, 1 equiv), DMF (20 mL), 1,2-dibromoethane (709 μL, 7.93 mmol, 1.5 equiv) and Cs₂CO₃ (5.17 g, 15.9 mmol, 3 equiv) at room temperature. The resulting mixture was stirred overnight at 60° C. under nitrogen atmosphere. The resulting mixture was quenched with water and extracted with EtOAc (3×10 mL). The combined organic layers were washed with brine (1×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 0% to 100% gradient in 20 min; detector, UV 254 nm. This resulted in tert-butyl (2S)-2-(7-chloro-1,1-dioxido-3,4-dihydro-2H-pyrido[3,2-b][1,4,5]oxathiazepin-2-yl)-3-(6-fluoro-2,3-dimethylphenyl)butanoate (2.3 g, 87.20%). Step 7: Synthesis of (2S)-2-(7-chloro-1,1-dioxido-3,4-dihydro-2H-pyrido[3,2-b][1,4,5]oxathiazepin-2-yl)-3-(6-fluoro-2,3-dimethylphenyl)butanoic acid

Into a 10 0 mL round-bottom flask were added tert-butyl (2S)-2-(7-chloro-1,1-dioxido-3,4-dihydro-2H-pyrido[3,2-b][1,4,5]oxathiazepin-2-yl)-3-(6-fluoro-2,3-dimethylphenyl)butanoate (2.3 g, 4.6 mmol, 1 equiv), DCM (19 mL) and TFA (6.25 mL) at room temperature. The resulting mixture was stirred for 1 h at room temperature. The resulting mixture was concentrated under vacuum. The residue was purified by reverse flash chromatography with the following conditions: column, C18 silica gel; mobile phase, MeCN in Water (0.1% FA), 0% to 100% gradient in 20 min; detector, UV 254 nm. This resulted in 2S)-2-(7-chloro-1,1-dioxido-3,4-dihydro-2H-pyrido[3,2-b][1,4,5]oxathiazepin-2-yl)-3-(6-fluoro-2,3-dimethylphenyl)butanoic acid (1.7 g, 83.3%).

### Step 8: Synthesis of 5-((1S,2R)-1-(7-chloro-1,1-dioxido-3,4-dihydro-2H-pyrido[3,2-b][1,4,5]oxathiazepin-2-yl)-2-(6-fluoro-2,3-dimethylphenyl)propyl)-1,3,4-oxadiazol-2 (3H)-one

Into a 40 mL vial were added 2S)-2-(7-chloro-1,1-dioxido-3,4-dihydro-2H-pyrido[3,2-b][1,4,5]oxathiazepin-2-yl)-3-(6-fluoro-2,3-dimethylphenyl) butanoic acid (100 mg, 0.23 mmol, 1 equiv), THF (1 mL) and CDI (168 mg, 1.04 mmol, 4.6 equiv) at room temperature. The resulting mixture was stirred 20 min at room temperature under air atmosphere. To the above mixture was added hydrazine hydrate (54.87 μL, 1.130 mmol, 5 equiv) dropwise at 0° C. The resulting mixture was stirred for additional 1 h at 0° C. The resulting mixture was quenched with water and extracted with EtOAc (2×5 mL). The combined organic layers were washed with brine (1×5 mL), dried over anhydrous Na₂SO₄. After filtration, the filtrate was concentrated under reduced pressure. Into a 10 mL vial were added the above crude product, dioxane (2 mL) and CDI (168 mg, 1.04 mmol, 4.6 equiv) at room temperature. The resulting mixture was stirred for 0.5h at room temperature under air atmosphere. The crude product (was purified by Chiral-Prep-HPLC with the following conditions: Column, XBridge Prep OBD C18 Column, 19*250 mm, 5 μm; mobile phase, undefined and undefined (50% undefined up to 60% in 10 min); Detector, UV254 nm. This resulted in Synthesis of 5-((1S,2R)-1-(7-chloro-1,1-dioxido-3,4-dihydro-2H-pyrido[3,2-b][1,4,5]oxathiazepin-2-yl)-2-(6-fluoro-2,3-dimethylphenyl)propyl)-1,3,4-oxadiazol-2 (3H)-one (13.8 mg, 12.3%). LCMS: (ES, m/z): [M+H]:483.10. ¹H NMR (400 MHz, DMSO-d₆) δ 8.50 (s, 1H), 7.86 (s, 1H), 7.03 (t, J=7.0 Hz, 1H), 6.83 (dd, J=12.2, 8.3 Hz, 1H), 5.43 (d, J=11.6 Hz, 1H), 4.59 (ddd, J=13.1, 8.4, 3.8 Hz, 1H), 4.30 (d, J=13.3 Hz, 1H), 3.83 (dp, J=19.2, 6.2, 5.6 Hz, 3H), 2.30 (s, 3H), 2.18 (s, 3H), 2.08 (s, 1H), 1.34 (d, J=6.9 Hz, 3H).

### Example 17: 5-((1S, 2R)-1-(7-chloro-1,1-dioxido-3,4-dihydro-2H-pyrido[4,3-b][1,4,5]oxathiazepin-2-yl)-2-(6-fluoro-2,3-dimethylphenyl)propyl)-1,3,4-oxadiazol-2 (3H)-one

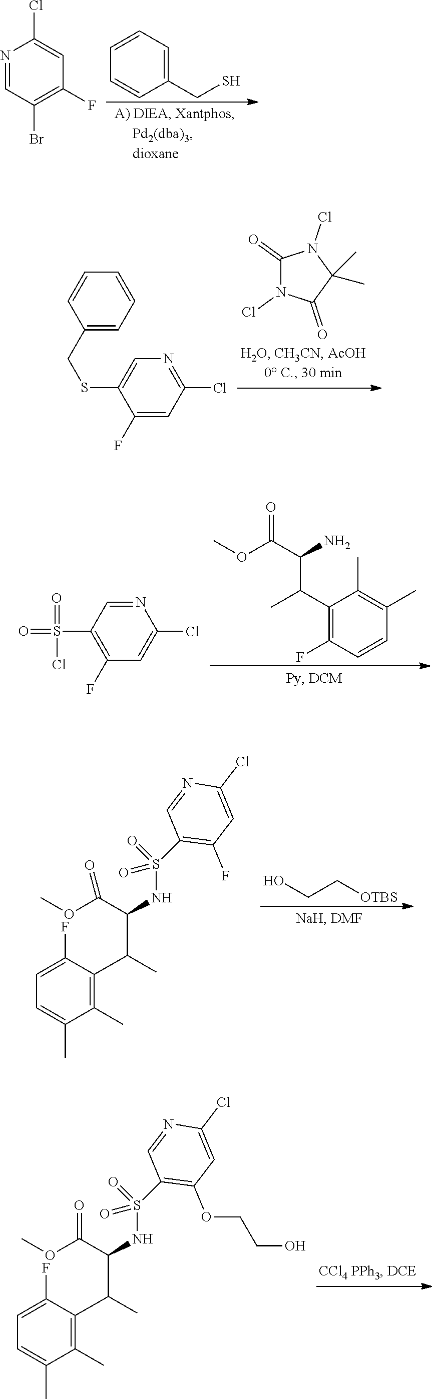

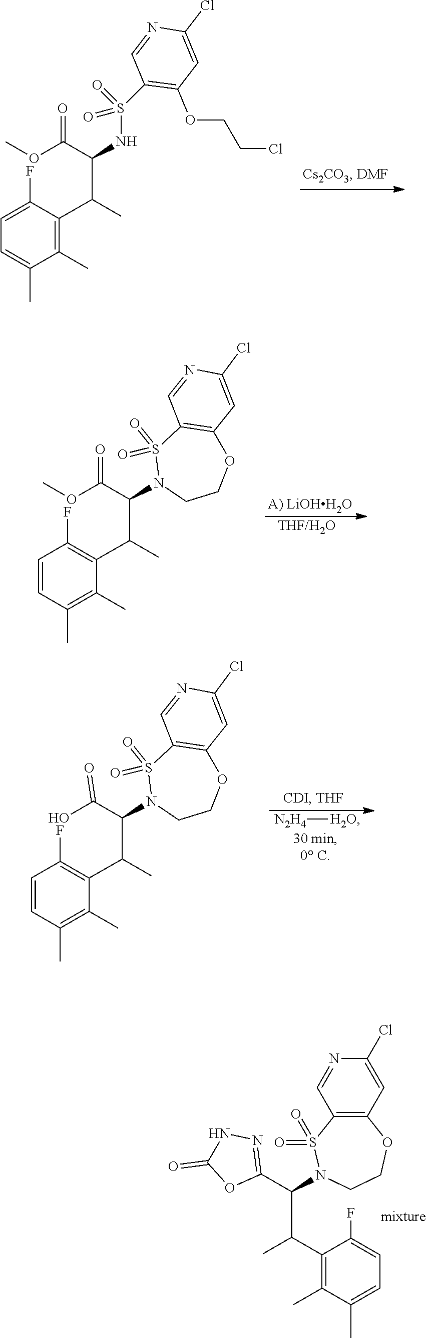

### Step 1: Synthesis of 5-(benzylthio)-2-chloro-4-fluoropyridine

To a stirred solution of 5-bromo-2-chloro-4-fluoropyridine (1 g, 4.75 mmol, 1 equiv) and dioxane (10 mL) was added DIEA (2.5 mL, 14.3 mmol, 3 equiv) Xantphos (550 mg, 0.95 mmol, 0.2 equiv) Pd₂(dba)₃ (435 mg, 0.48 mmol, 0.1 equiv)benzyl mercaptan (669 μL, 5.7 mmol, 1.2 equiv) at room temperature. The resulting mixture was stirred for 4 h at 100° C. under nitrogen atmosphere. The reaction was quenched with water at room temperature. The resulting mixture was extracted with EtOAc (3×50 mL). The combined organic layers were washed with brine (2×200 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (10 mmol/L NH₄HCO₃), 10% to 50% gradient in 20 min; detector, UV 220 nm. This resulted in 5-(benzylthio)-2-chloro-4-fluoropyridine (550 mg, 45.62%).

### Step 2: Synthesis of 6-chloro-4-fluoropyridine-3-sulfonyl chloride

To a stirred solution of 5-(benzylthio)-2-chloro-4-fluoropyridine (1.34 g, 5.28 mmol, 1 equiv) and H₂O (856 μl) AcOH (1.5 mL, 26 mmol, 5 equiv) in MeCN was added 1,3-dichloro-5,5-dimethylimidazolidine-2,4-dione (2.08 g, 10.6 mmol, 2 equiv) in portions at 0° C. The resulting mixture was stirred for 30 min at 0° C. The reaction was quenched with Water/Ice at room temperature. The resulting mixture was extracted with EtOAc (2×10 mL). The combined organic layers were washed with brine (2×20 mL), dried over anhydrous Na₂SO₄. After filtration, the filtrate was concentrated under reduced pressure.

The crude product was used in the next step directly without further purification.

### Step 3: Synthesis of methyl (2S)-2-((6-chloro-4-fluoropyridine)-3-sulfonamido)-3-(6-fluoro-2,3-dimethylphenyl) butanoate

To a stirred solution of methyl (2S)-2-amino-3-(6-fluoro-2,3-dimethylphenyl) butanoate(1.7 g, 7 mmol, 1 equiv) and Pyridine (2.8 mL, 35 mmol, 5 equiv) was added 6-chloro-4-fluoropyridine-3-sulfonyl chloride (1.6 g, 7 mmol, 1 equiv) DCM (20 mL) dropwise at 0° C. The resulting mixture was stirred for overnight at room temperature. The resulting mixture was extracted with DCM (3×20 mL). The combined organic layers were washed with brine (3×50 mL), dried over anhydrous MgSO₄. After filtration, the filtrate was concentrated under reduced pressure. The residue was purified by silica gel column chromatography, eluted with PE/EtOAc (9:1) to afford methyl (2S)-2-((6-chloro-4-fluoropyridine)-3-sulfonamido)-3-(6-fluoro-2,3-dimethylphenyl) butanoate (1.56 g, 51.8%).

### Step 4: Synthesis of methyl (2S)-2-((6-chloro-4-(2-hydroxyethoxy) pyridine)-3-sulfonamido)-3-(6-fluoro-2,3-dimethylphenyl) butanoate

To a stirred solution of methyl (2S)-2-(6-chloro-4-fluoropyridine-3-sulfonamido)-3-(6-fluoro-2,3-dimethylphenyl) butanoate (710 mg, 1.64 mmol, 1 equiv) and 2-[(tert-butyldimethylsilyl)oxy]ethanol (650 μL, 3.28 mmol, 2.0 equiv) in DMF was added NaH (197 mg, 4.92 mmol, 3 equiv, 60%) in portions at 0° C. The resulting mixture was stirred for 3 h at room temperature. The reaction was quenched with water at room temperature. The residue was purified by reverse flash chromatography with the following conditions: column, C18 silica gel; mobile phase, MeCN in Water (0.1% FA), 0% to 100/a gradient in 30 min; detector, UV 220 nm. This resulted methyl (2S)-2-((6-chloro-4-(2-hydroxyethoxy) pyridine)-3-sulfonamido)-3-(6-fluoro-2,3-dimethylphenyl) butanoate (190 mg, 24.4%).

### Step 5: Synthesis of methyl (2S)-2-((6-chloro-4-(2-chloroethoxy) pyridine)-3-sulfonamido)-3-(6-fluoro-2,3-dimethylphenyl) butanoate

To a stirred solution of methyl (2S)-2-((6-chloro-4-(2-hydroxyethoxy) pyridine)-3-sulfonamido)-3-(6-fluoro-2,3-dimethylphenyl) butanoate (180 mg, 0.38 mmol, 1 equiv) and PPh₃ (497 mg, 1.9 mmol, 2 equiv) in DCE (6 mL) was added CCl₄ (137 μL, 1.42 mmol, 1.5 equiv) dropwise at room temperature. The resulting mixture was stirred for 1 h at 80° C. The resulting mixture was concentrated under reduced pressure. The residue was purified by reverse flash chromatography with the following conditions: column, C18 silica gel; mobile phase, MeCN in Water (10 mmol/L NH₄HCO₃), 0% to 10 0% gradient in 30 min; detector, UV 220 nm. This resulted in methyl (2S)-2-((6-chloro-4-(2-chloroethoxy) pyridine)-3-sulfonamido)-3-(6-fluoro-2,3-dimethylphenyl) butanoate (160 mg, 85.6%).

### Step 6: Synthesis of methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoate

To a stirred solution of methyl (2S)-2-[6-chloro-4-(2-chloroethoxy) pyridine-3-sulfonamido]-3-(6-fluoro-2,3-dimethylphenyl) butanoate (260 mg, 0.53 mmol, 1 equiv) in DMF was added Cs₂CO₃ (343 mg, 1.05 mmol, 2 equiv) at room temperature. The resulting mixture was stirred 1 h at 60° C. The resulting mixture was extracted with EtOAc (2×50 mL). The combined organic layers were washed with brine (2×50 mL), dried over anhydrous Na₂SO₄. After filtration, the filtrate was concentrated under reduced pressure. The crude product (mg) was purified by Prep-HPLC with the following conditions (PE:EtOAc 1:1) to afford methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoate (190 mg, 78.91%).

### Step 7: Synthesis of (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoic acid

To a stirred solution methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl) butanoate (20 mg, 0.41 mmol, 1 equiv) and H₂O (1 mL) in THF was added LiOH·H₂O (87 mg, 2.08 mmol, 5 equiv) at room temperature. The resulting mixture was stirred 30 min at room temperature. The mixture was acidified to pH 6 with conc. HCl. The resulting mixture was extracted with EtOAc (3×20 mL). The combined organic layers were washed with brine (2×50 mL), dried over anhydrous Na₂SO₄. After filtration, the filtrate was concentrated under reduced pressure. The crude product/resulting mixture was used in the next step directly without further purification.

### Step 8: Synthesis of 5-((1S)-1-(7-chloro-1,1-dioxido-3,4-dihydro-2H-pyrido[4,3-b][1,4,5]oxathiazepin-2-yl)-2-(6-fluoro-2,3-dimethylphenyl)propyl)-1,3,4-oxadiazol-2 (3H)-one

To a stirred solution of 2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoic acid (45 mg, 0.10 mmol, 1 equiv) and CDI (57.7 mg, 0.36 mmol, 3.5 equiv) in THF at room temperature. The resulting mixture was stirred for 30 min at room temperature. To the above mixture was added NH₂NH₂·H₂O (14.8 μL, 0.31 mmol, 3 equiv) dropwise at 0° C. The resulting mixture was stirred for additional 30 min at 0° C. The reaction was quenched with water/Ice at room temperature. The resulting mixture was extracted with EtOAc (2×50 mL).

The combined organic layers were washed with brine (2×50 mL), dried over anhydrous Na₂SO₄. After filtration, the filtrate was concentrated under reduced pressure. To the above mixture was added CDI (57.7 mg, 0.36 mmol, 3.5 equiv) dioxane (1 mL) in portions at room temperature. The resulting mixture was stirred for additional 30 min at room temperature. The reaction was quenched with water at room temperature. The residue was purified by reverse flash chromatography with the following conditions: Column: XBridge Prep OBD CIS Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 24% B to 52% B in 9 min, 52% B; Wave Length: 220 nm; RT1 (min): 7. This resulted in 5-((1S)-1-(7-chloro-1,1-dioxido-3,4-dihydro-2H-pyrido[4,3-b][1,4,5]oxahiazepin-2-yl)-2-(6-fluoro-2,3-dimethylphenyl)propyl)-1,3,4-oxadiazol-2 (3H)-one (0.3 mg, 16.7%). LCMS:(ES, m/z): [M−H]=480.90. ¹H NMR (300 MHz, Methanol-d₄) δ 8.67-8.42 (m, 1H), 7.08-6.97 (m, 2H), 6.87-6.44 (ddd, J=75.1, 12.0, 9.2 Hz, 1H), 5.57-5.51 (dd, J=11.7, 6.5 Hz, 1H), 4.66-4.62 (q, J=6.3, 5.3 Hz, 1H), 4.48-4.46 (m, 1H), 4.03-3.95 (d, J=14.6 Hz, 1H), 3.87-3.79 (m, 1H), 3.63-3.55 (m, 1H), 2.4-2.35 (d, J=14.8 Hz, 3H), 2.30-2.21 (d, J=5.2 Hz, 3H), 1.60-1.50 (d, J=7.0 Hz, 1H), 1.30-1.20 (d, J=6.8 Hz, 2H).

### Example 18: methyl 7-chloro-2-((1S,2R)-2-(6-fluoro-2,3-dimethylphenyl)-1-(5-oxo-4,5-dihydro-1,3,4-oxadiazol-2-yl) propyl)-3,4-dihydro-2H-benzo[b][1,4,5]oxathiazepine-9-carboxylate 1,1-dioxide

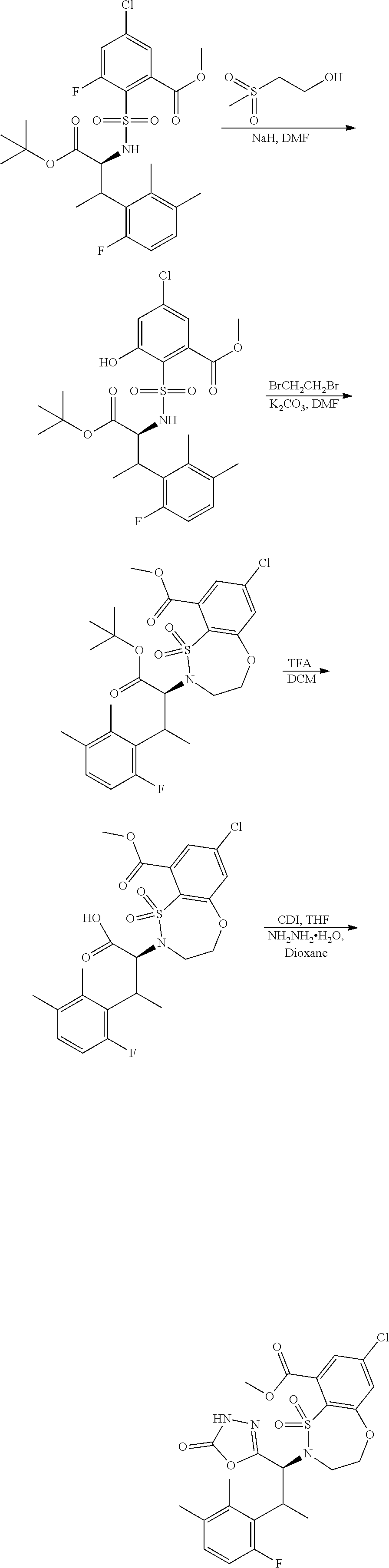

### Step 1: Synthesis of methyl 2-(N-((2S)-1-(tert-butoxy)-3-(6-fluoro-2,3-dimethylphenyl)-1-oxobutan-2-yl) sulfamoyl)-5-chloro-3-hydroxybenzonte

To a stirred solution of methyl 2-(N-((2S)-1-(tert-butoxy)-3-(6-fluoro-2,3-dimethylphenyl)-1-oxobutan-2-yl) sulfamoyl)-5-chloro-3-fluorobenzoate (2.14 g, 4.02 mmol, 1 equiv) see ex-19) and 2-methanesulfonylethanol (749 mg, 6.03 mmol, 1.5 equiv) in DMF were added NaH (402 mg, 10.06 mmol, 2.5 equiv, 60%) in portions at 0° C. The resulting mixture was stirred for 1 h at room temperature. The reaction was quenched by the addition of AcOH at room temperature. The residue was purified by reverse flash chromatography with the following conditions: column, C18 silica gel; mobile phase, MeCN in Water (10 mmol/L NH₄HCO₃), 0% to 100% gradient in 40 min; detector, UV 220 nm. This resulted in methyl 2-(N-((2S)-1-(tert-butoxy)-3-(6-fluoro-2,3-dimethylphenyl)−1-oxobutan-2-yl) sulfamoyl)-5-chloro-3-hydroxybenzoate (470 mg, 22.1%).

### Step 2: Synthesis of methyl 2-((2S)-1-(tert-butoxy)-3-(6-fluoro-2,3-dimethylphenyl)-1-oxobutan-2-yl)-7-chloro-3,4-dihydro-2H-benzo[b][1,4,5]oxathiazepine-9-carboxylate 1,1-dioxide

To a stirred solution of methyl 2-(N-((2S)-1-(tert-butoxy)-3-(6-fluoro-2,3-dimethylphenyl)-1-oxobutan-2-yl) sulfamoyl)-5-chloro-3-hydroxybenzoate (458 mg, 0.86 mmol, 1 equiv) and dibromoethane (74.5 μL, 0.86 mmol, 1 equiv) in DMF was added K₂CO₃ (358 mg, 2.59 mmol, 3 equiv) at room temperature. The resulting mixture was stirred 1 h at 60° C. The residue was purified by reverse flash chromatography with the following conditions: column, C18 silica gel; mobile phase, MeCN in Water (0.1% FA), 0% to 100% gradient in 30 min; detector, UV 220 nm. This resulted methyl 2-((2S)-1-(tert-butoxy)-3-(6-fluoro-2,3-dimethylphenyl)-1-oxobutan-2-yl)-7-chloro-3,4-dihydro-2H-benzo[b][1,4,5]oxathiazepine-9-carboxylate 1,1-dioxide (290 mg, 60.4%).

### Step 3: Synthesis of (2S)-2-(7-chloro-9-(methoxycarbonyl)-1,1-dioxido-3,4-dihydro-2H-benzo[b][1,4,5]oxathiazepin-2-yl)-3-(6-fluoro-2,3-dimethylphenyl)butanoic acid

To a stirred solution of methyl 2-((2S)-1-(tert-butoxy)-3-(6-fluoro-2,3-dimethylphenyl)-1-oxobutan-2-yl)-7-chloro-3,4-dihydro-2H-benzo[b][1,4,5]oxathiazepine-9-carboxylate 1,1-dioxide (270 mg, 0.49 mmol, 1 equiv) in 1 ml DCM was added TFA (1 mL) at room temperature. The resulting mixture was stirred 30 min at room temperature. The resulting mixture was concentrated under reduced pressure. The residue was purified by reverse flash chromatography with the following conditions: column, C18 silica gel; mobile phase, MeCN in Water (0.1% FA), 0/a to 100% gradient in 20 min; detector, UV 254 nm. This resulted in (2S)-2-(7-chloro-9-(methoxycarbonyl)-1,1-dioxido-3,4-dihydro-2H-benzo[b][1,4,5]oxathiazepin-2-yl)-3-(6-fluoro-2,3-dimethylphenyl)butanoic acid (230 mg, 94.75%).

### Step 4: Synthesis of: methyl 7-chloro-2-((1S,2R)-2-(6-fluoro-2,3-dimethylphenyl)-1-(5-oxo-4,5-dihydro-1,3,4-oxadiazol-2-yl) propyl)-3,4-dihydro-2H-benzo[b][1,4,5]oxathiazepine-9-carboxylate 1,1-dioxide

To a stirred solution of (2S)-2-[7-chloro-9-(methoxycarbonyl)-1,1-dioxo-3,4-dihydro-5,1lambda6,2-benzoxathiazepin-2-yl]-3-(6-fluoro-2,3-dimethylphenyl) butanoic acid (35 mg, 0.070 mmol, 1 equiv) and CDI (17.03 mg, 0.105 mmol, 1.5 equiv) in THF at room temperature. The resulting mixture was stirred for 30 min at room temperature. To the above mixture was added NH₂NH₂·H₂O (10.21 μL, 0.210 mmol, 3 equiv) dropwise at 0° C. The resulting mixture was stirred for additional 30 min at 0° C. The reaction was quenched with Water at room temperature. The resulting mixture was extracted with EtOAc (2×20 mL). The combined organic layers were washed with brine (3×20 mL), dried over anhydrous Na₂SO₄. After filtration, the filtrate was concentrated under reduced pressure. To the above mixture was added Dioxane (1 mL) CDI (28.38 mg, 0.175 mmol, 2.5 equiv) in portions at room temperature.

The resulting mixture was stirred for additional 1 h at room temperature. The reaction was quenched with Water at room temperature. The residue was purified by reverse flash chromatography with the following conditions: Column: XBridge Prep OBD C18 Column, 30*150 mm, 5 μm; Mobile Phase A: 10 mmol NH₄HCO₃+0.05% NH₃H₂O, Mobile Phase B: ACN; Flow rate: 60 mL/min; Gradient: 20% B to 46% B in 12 min, 46% B; Wave Length: 254/220 nm; RT1 (min): 6.88. This resulted in methyl 7-chloro-2-[(1S)-2-(6-fluoro-2,3-dimethylphenyl)-1-(5-oxo-4H-1,3,4-oxadiazol-2-yl)propyl]-1,1-dioxo-3,4-dihydro-5,1lambda6,2-benzoxathiazepine-9-carboxylate (10 mg, 25.95%). LC-MS: (ES, m/z): [M−H]=538.15. ¹H NMR (300 MHz, Methanol-d₄) δ 7.33 (d, J=2.4 Hz, 1H), 7.19-7.13 (m, 1H), 7.03-6.67 (m, 2H), 5.4-5.45 (t, J=11.5 Hz, 1H), 4.85-4.75 (d, J=12.4 Hz, 1H), 4.46-4.42 (t, J=13.5 Hz, 1H), 4.09-3.76 (m, 6H), 2.45-2.34 (d, J=12.2 Hz, 3H), 2.30-2.21 (d, J=6.0 Hz, 3H), 1.43-1.40 (d, J=6.9 Hz, 2H), 1.25-1.23 (d, J=7.0 Hz, 1H).

### Example 19: methyl 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylate 1,1-dioxide

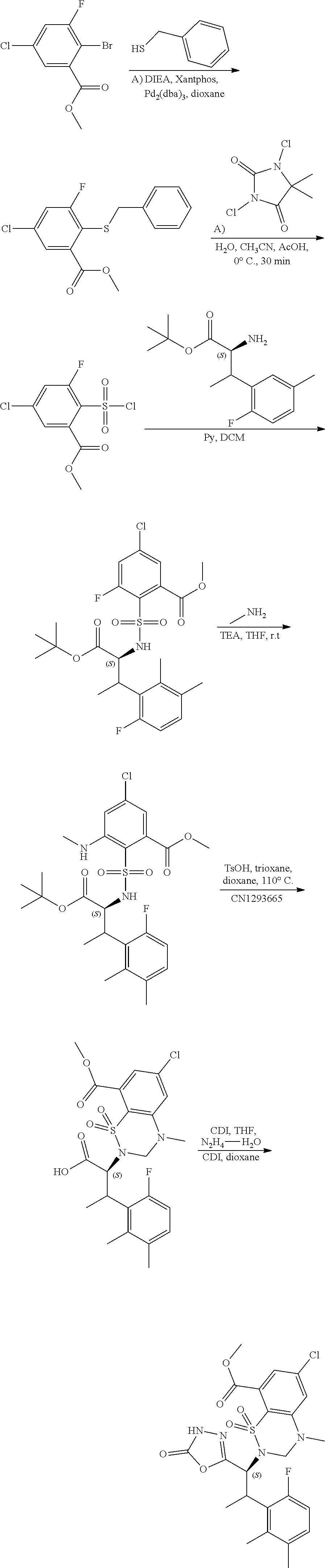

### Step 1: Synthesis of methyl 2-(benzylthio)-5-chloro-3-fluorobenzonte

Into a 250 mL round-bottom flask were added methyl 2-bromo-5-chloro-3-fluorobenzoate (4.7 g, 17.5 mmol, 1 equiv) and dioxane (50 mL) at room temperature. To the above mixture was added DIEA (9.2 mL, 52.7 mmol, 3 equiv), Xantphos (43.3 mg, 0.075 mmol, 0.2 equiv), Pd₂(dba)₃ (34.2 mg, 0.037 mmol, 0.1 equiv), benzyl mercaptan (50.5 μL, 0.430 mmol, 1.15 equiv) dropwise at room temperature. The resulting mixture was stirred overnight at 100° C. under nitrogen atmosphere. The reaction was quenched by the addition of water at room temperature. The resulting mixture was filtered. The filter cake was washed with EtOAc. The filtrate was extracted with EtOAc. The combined organic layers were washed with brine, dried over anhydrous Na₂SO₄. After filtration, the filtrate was concentrated under reduced pressure. The residue was purified by Prep-TLC (PEI/EtOAc 5:1) to afford methyl 2-(benzylthio)-5-chloro-3-fluorobenzoate (4.4 g, 80.6%/).

### Step 2: Synthesis of methyl 5-chloro-2-(chlorosulfonyl)-3-fluorobenzonte

To a stirred mixture of methyl 2-(benzylthio)-5-chloro-3-fluorobenzoate (3 g, 9.6 mmol, 1 equiv) and AcOH (2.77 mL, 48.3 mmol, 5 equiv) H₂O (1.57 mL, 86.9 mmol, 9 equiv) in MeCN was added 1,3-dichloro-5,5-dimethylimidazolidine-2,4-dione (3.80 g, 19.3 mmol, 2 equiv) in portions at 0° C.

The resulting mixture was stirred for 30 min at 0° C. The reaction was quenched with water at room temperature. The resulting mixture was extracted with EtOAc (3×50 mL). The combined organic layers were washed with brine (2×200 mL), dried over anhydrous Na₂SO₄. After filtration, the filtrate was concentrated under reduced pressure. The residue was purified by silica gel column chromatography, eluted with PEI/EtOAc (9:1) to afford methyl 5-chloro-2-(chlorosulfonyl)-3-fluorobenzoate (2.45 g, 88.4%).

### Step 3: Synthesis of methyl 2-(N-((2S)-1-(tert-butoxy)-3-(6-fluoro-2,3-dimethylphenyl)-1-oxobutan-2-yl) sulfamoyl)-5-chloro-3-fluorobenzoate

To a stirred solution of tert-butyl (2S)-2-amino-3-(6-fluoro-2,3-dimethylphenyl) butanoate (2.41 g, 8.562 mmol, 1 equiv) and Pyridine (3.46 mL, 42.8 mmol, 5 equiv) in DCM were added methyl 5-chloro-2-(chlorosulfonyl)-3-fluorobenzoate (2.46 g, 8.56 mmol, 1 equiv) in DCM (30 mL) dropwise at 0° C. The resulting mixture was stirred overnight at room temperature. The resulting mixture was extracted with DCM (2×50 mL). The combined organic layers were washed with brine (2×30 mL), dried over anhydrous MgSO₄. After filtration, the filtrate was concentrated under reduced pressure. The residue was purified by silica gel column chromatography, eluted with PE/EtOAc (9:1) to afford methyl 2-(N-((2S)-1-(tert-butoxy)-3-(6-fluoro-2,3-dimethylphenyl)-1-oxobutan-2-yl) sulfamoyl)-5-chloro-3-fluorobenzoate (3.45 g, 75.7%).

### Step 4: Synthesis of methyl 2-(N-((2S)-1-(tert-butoxy)-3-(6-fluoro-2,3-dimethylphenyl)-1-oxobutan-2-yl) sulfamoyl)-5-chloro-3-(methylamino)benzoate

To a stirred solution of methyl 2-(N-((2S)-1-(tert-butoxy)-3-(6-fluoro-2,3-dimethylphenyl)-1-oxobutan-2-yl) sulfamoyl)-5-chloro-3-fluorobenzoate (1.4 g, 2.63 mmol, 1 equiv) in THF (15 mL) were added TEA (3.66 mL, 26.3 mmol, 10 equiv) and methylamine (6.58 mL, 13.2 mmol, 5 equiv) at room temperature. The resulting mixture was stirred 6 h at room temperature. The resulting mixture was extracted with EtOAc (2×20 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EtOAc (9:1) to afford methyl 2-(N-((2S)-1-(tert-butoxy)-3-(6-fluoro-2,3-dimethylphenyl)-1-oxobutan-2-yl) sulfamoyl)-5-chloro-3-(methylamino) benzoate (495 mg, 34.6%).

### Step 5: Synthesis of (2S)-2-(6-chloro-8-(methoxycarbonyl)-4-methyl-1,1-dioxido-3,4-dihydro-2H-benzo[e][1,2,4]thiadiazin-2-yl)-3-(6-fluoro-2,3-dimethylphenyl)butanoic acid

To a stirred mixture of methyl 2-(N-((2S)-1-(tert-butoxy)-3-(6-fluoro-2,3-dimethylphenyl)-1-oxobutan-2-yl) sulfamoyl)-5-chloro-3-(methylamino) benzoate (360 mg, 0.66 mmol, 1 equiv) in dioxane (14 mL) were added TsOH (114 mg, 0.66 mmol, 1 equiv) and 1,3,5-trioxane (597 mg, 6.63 mmol, 10 equiv) in portions at room temperature. The resulting mixture was stirred overnight at 110° C. The resulting mixture was concentrated under vacuum. The residue was purified by reverse flash chromatography with the following conditions: column, C18 silica gel; mobile phase, MeCN in Water (0.1% FA), 10% to 80% gradient in 20 min; detector, UV 254 nm. This resulted of (2S)-2-(6-chloro-8-(methoxycarbonyl)-4-methyl-1,1-dioxido-3,4-dihydro-2H-benzo[e][1,2,4]thiadiazin-2-yl)-3-(6-fluoro-2,3-dimethylphenyl)butanoic acid (300 mg, 90.7%).

### Step 6: Synthesis of methyl 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylate 1,1-dioxide

Into a 50 mL round-bottom flask were added (2S)-2-(6-chloro-8-(methoxycarbonyl)-4-methyl-1,1-dioxido-3,4-dihydro-2H-benzo[e][1,2,4] thiadiazin-2-yl)-3-(6-fluoro-2,3-dimethylphenyl)butanoic acid (387 mg, 0.78 mmol, 1 equiv) in THF (4 mL). To the above mixture was added CDI (189 mg, 1.16 mmol, 1.5 equiv) in portions at room temperature. The resulting mixture was stirred for additional 30 min at room temperature. To the above mixture was added N₂H₄·H₂O (113 μL, 2.33 mmol, 3 equiv) dropwise at 0° C.

The resulting mixture was stirred for additional 30 min at 0° C. The reaction was quenched with water at 0° C. The resulting mixture was extracted with EtOAc (2×5 mL). The combined organic layers were washed with brine (1×10 mL), dried over anhydrous Na₂SO₄. After filtration, the filtrate was concentrated under reduced pressure to afford crude product SM1. Into a 50 mL round-bottom flask were added SM1 in dioxane (4 mL). To the above mixture was added CDI (314.42 mg, 1.940 mmol, 2.5 equiv) in portions at room temperature. The resulting mixture was stirred for additional 1 h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in methyl 6-chloro-2-((1S)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylate 1,1-dioxide (360 mg, 86.1%).

The product (60 mg) was further purified by Prep-HPLC with the following conditions ((Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 220 nm; RT1 (min): 7) to afford methyl 6-chloro-2-methyl 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylate 1,1-dioxide (20.6 mg, 33.9%). LC-MS: (ES, m/z): [M−H]⁺=537.15. ¹H NMR (300 MHz, Methanol-d₄) δ 6.98-6.92 (d, J=19.9 Hz, 2H), 6.83 (s, 1H), 6.71 (s, 1H), 5.43-5.40 (d, J=13.3 Hz, 2H), 5.09-5.04 (d, J=15.1 Hz, 1H), 3.94-3.88 (d, J=4.6 Hz, 4H), 2.93 (s, 3H), 2.37 (d, J=4.0 Hz, 3H), 2.22 (d, J=4.1 Hz, 3H), 1.43 (s, 3H).

### Example 20: 5-((1S,2R)-1-(8-chloro-5,5-dioxido-1,2,3,3a-tetrahydro-4H-benzo[e]pyrrolo[2,1-c][1,2,4]thiadiazin-4-yl)-2-(6-fluoro-2,3-dimethylphenyl)propyl)-1,3,4-oxadiazol-2 (3H)-one

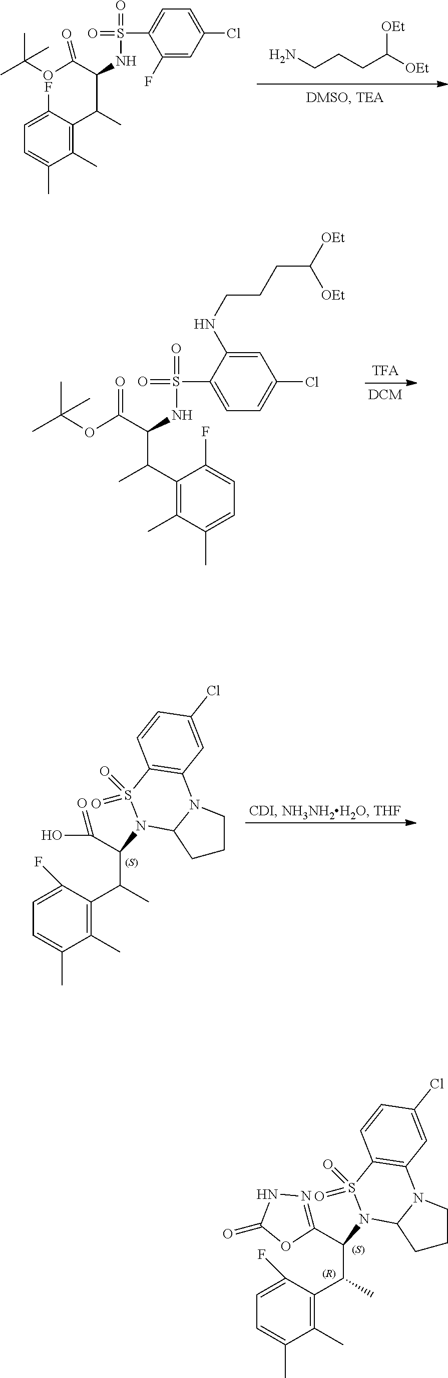

### Step 1: Synthesis of tert-butyl (2S)-2-((4-chloro-2-((4,4-diethoxybutyl) amino) phenyl)sulfonamido)-3-(6-fluoro-2,3-dimethylphenyl)butanoate

Into a 40 mL round-bottom flask were added tert-butyl (2S)-2-(4-chloro-2-fluorobenzenesulfonamido)-3-(6-fluoro-2,3-dimethylphenyl) butanoate (see example 4) (1 g, 2.1 mmol, 1 equiv),4,4-diethoxy-butylamine (4.76 g, 29.5 mmol, 14 equiv), TEA (3 g, 29.5 mmol, 14 equiv) and DMSO (20 mL) at room temperature. The resulting mixture was stirred for overnight at 80° C. under air atmosphere. The residue was purified by reverse flash chromatography with the following conditions: column, C18 silica gel; mobile phase, MeCN in Water (0.1% FA), 5% to 100% gradient in 30 min; detector, UV 220 nm, to afford tert-butyl (2S)-2-((4-chloro-2-((4,4-diethoxybutyl) amino) phenyl) sulfonamide)-3-(6-fluoro-2,3-dimethylphenyl) butanoate (1 g, 77%).

### Step 2: Synthesis of (2S)-2-(8-chloro-5,5-dioxido-1,2,3,3a-tetrahydro-4H-benzo[e]pyrrolo[2,1-c][1,2,4]thiadiazin-4-yl)-3-(6-fluoro-2,3-dimethylphenyl)butanoic acid

Into a 8 mL round-bottom flask were added tert-butyl (2S)-2-{4-chloro-2-[(4,4-diethoxybutyl) amino]benzenesulfonamido}-3-(6-fluoro-2,3-dimethylphenyl) butanoate (200 mg, 0.33 mmol, 1 equiv), TFA (1 mL) and DCM (3 mL) at room temperature. The resulting mixture was stirred overnight at room temperature under air atmosphere. The resulting mixture was concentrated under reduced pressure. The residue was purified by reverse flash chromatography with the following conditions: column, C18 silica gel; mobile phase, MeCN in Water (0.1% FA), 5% to 100% gradient in 30 min; detector, UV 220 nm, to (2S)-2-(8-chloro-5,5-dioxido-1,2,3,3a-tetrahydro-4H-benzo[e]pyrrolo[2,1-c][1,2,4]thiadiazin-4-yl)-3-(6-fluoro-2,3-dimethylphenyl)butanoic acid (100 mg, 65.9%) as a light yellow oil.

### Step 3: Synthesis of 5-((1S,2R)-1-(8-chloro-5,5-dioxido-1,2,3,3a-tetrahydro-4H-benzo[e]pyrrolo[2,1-c][1,2,4]thiadiazin-4-yl)-2-(6-fluoro-2,3-dimethylphenyl)propyl)-1,3,4-oxadiazol-2 (3H)-one

Into a 50 mL round-bottom flask were added (2S)-2-(8-chloro-5,5-dioxido-1,2,3,3a-tetrahydro-4H-benzo[e]pyrrolo[2,1-c][1,2,4]thiadiazin-4-yl)-3-(6-fluoro-2,3-dimethylphenyl)butanoic acid (200 mg, 0.44 mmol, 1 equiv), CDI (108 mg, 0.67 mmol, 2.6 equiv) and THF (2 mL) at room temperature, the resulting mixture was stirred 30 min at room temperature. the mixture was added hydrazine (24.7 mg, 0.77 mmol, 3 equiv) dropwise at 0° C. The resulting mixture was stirred 30 min at 0° C. The reaction was quenched with water at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure, added dioxane (2 mL) and CDI (108 mg, 0.67 mmol, 2.6 equiv) at room temperature. The resulting mixture was stirred f 30 min at room temperature and then poured in water and extracted with EtOAc (3×20 mL). The combined organic layers were washed with brine (2×10 mL), dried over anhydrous Na₂SO₄. The residue was purified by reverse flash chromatography with the following conditions: column, C18 silica gel; mobile phase, MeCN in water (0.1% FA), 5% to 100% gradient in 30 min; detector, UV 220 nm, The product was further purified by Chiral-Prep-HPLC with the following conditions: Column, XBridge Prep OBD C18 Column, 30*150 mm, 5 μm; mobile phase, Water(10 mmol/L NH₄HCO₃+0.1% NH₃·H₂O) and ACN (35% ACN up to 65% in 8 min); Detector, UV 220, to afford of 5-((1S,2R)-1-(8-chloro-5,5-dioxido-1,2,3,3a-tetrahydro-4H-benzo[e]pyrrolo[2,1-c][1,2,4]thiadiazin-4-yl)-2-(6-fluoro-2,3-dimethylphenyl)propyl)-1,3,4-oxadiazol-2 (3H)-one (14 mg, 10.6%). ¹H NMR (300 MHz, Methanol-d₄) δ 7.55 (dd, J=21.3, 8.5 Hz, 1H), 7.01-6.91 (m, 1H), 6.80-6.66 (m, 2H), 6.61-6.43 (m, 1H), 5.97-5.64 (m, 1H), 4.92 (d, J=3.0 Hz, 1H), 4.44 (dd, J=12.1, 6.9 Hz, 1H), 3.56-3.31 (m, 2H), 3.07 (s, 1H), 2.70-2.36 (m, 2H), 2.33 (s, 1H), 2.27 (s, 2H), 2.21 (s, 1H), 2.18 (s, 2H), 2.11 (d, J=12.7 Hz, 1H), 2.03 (s, 1H), 1.50 (dd, J=7.0, 1.3 Hz, 3H).

LCMS:(ES, m/z): [M+H]:507.15.

### Example 21: 5-((1S,2R)-1-(7-chloro-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

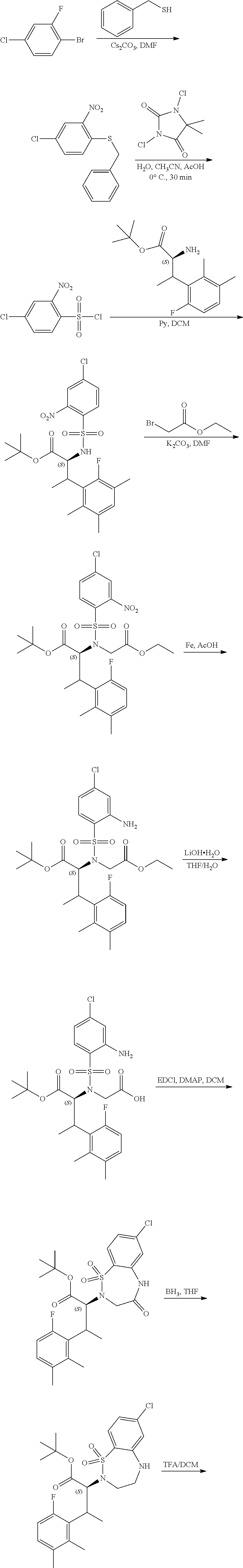

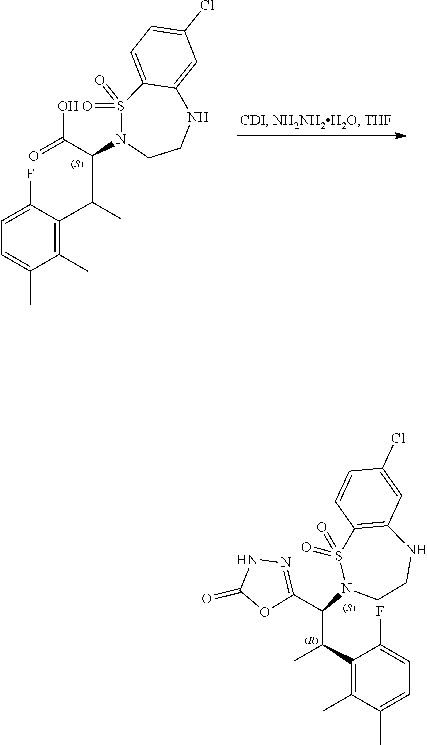

### Step 1: Synthesis of benzyl(4-chloro-2-nitrophenyl) sulfane

To a stirred solution of 1-bromo-4-chloro-2-nitrobenzene (50 g, 211 mmol, 1 equiv) and DMF (500 mL) was added Cs₂CO₃ (207 g, 634 mmol, 3.00 equiv) benzyl mercaptan (30 mL, 254 mmol, 1.2 equiv) dropwise at room temperature. The resulting mixture was stirred overnight at room temperature. The reaction was quenched with water at room temperature. The resulting mixture was extracted with EtOEt (3×1000 mL). The combined organic layers were washed with brine (500 mL), dried over anhydrous Na₂SO₄.

After filtration, the filtrate was concentrated under reduced pressure. The crude product was re-crystallized from PE/ethyl acetate (10:1) to benzyl(4-chloro-2-nitrophenyl) sulfane (30 g, 50.7%).

### Step 2: Synthesis of 4-chloro-2-nitrobenzenesulfonyl chloride

To a stirred solution of benzyl(4-chloro-2-nitrophenyl) sulfane (30 g, 107 mmol, 1 equiv) and H₂O (20 ml) in acetonitrile was added AcOH (28 mL) in portions at room temperature. To the above mixture was added 1,3-dichloro-5,5-dimethylimidazolidine-2,4-dione (42.3 g, 215 mmol, 2 equiv) in portions over 10 min at 0° C. The resulting mixture was stirred for additional 30 min at 0° C. The reaction was quenched with Water/Ice. The resulting mixture was extracted with EtOAc (3×300 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EtOAc (10:1) to afford 4-chloro-2-nitrobenzenesulfonyl chloride (30 g, crude).

### Step 3: Synthesis of tert-butyl (2S)-2-((4-chloro-2-nitrophenyl) sulfonamido)-3-(6-fluoro-2,3-dimethylphenyl)butanoate

To a stirred solution of tert-butyl (2S)-2-amino-3-(6-fluoro-2,3-dimethylphenyl)butanoate (5.10 g, 18.1 mmol, 0.8 equiv) and pyridine (18.3 mL, 227 mmol, 10 equiv) in DCM was added 4-chloro-2-nitrobenzenesulfonyl chloride (5.8 g, 22.7 mmol, 1 equiv) in DCM dropwise at 0° C. The resulting mixture was stirred overnight at room temperature. The resulting mixture was concentrated under vacuum. The residue was purified by silica gel column chromatography, eluted with PE/EtOAc (10:1) to afford tert-butyl (2S)-2-((4-chloro-2-nitrophenyl) sulfonamido)-3-(6-fluoro-2,3-dimethylphenyl) butanoate (6.2 g, 54.6%).

### Step 4: Synthesis of tert-butyl (2S)-2-((4-chloro-N-(2-ethoxy-2-oxoethyl)-2-nitrophenyl) sulfonamido)-3-(6-fluoro-2,3-dimethylphenyl) butanoate

To a stirred solution of afford tert-butyl (2S)-2-((4-chloro-2-nitrophenyl) sulfonamido)-3-(6-fluoro-2,3-dimethylphenyl) butanoate (1.8 g, 3.6 mmol, 1 equiv) and K₂CO₃ (1 g, 7.2 mmol, 2 equiv) in DMF was added ethyl bromoacetate (400 μL, 3.6 mmol, 1 equiv) in portions at room temperature. The resulting mixture was stirred for 60 min at 60° C. The mixture was allowed to cool down to room temperature. The reaction was quenched with water at room temperature. The resulting mixture was extracted with EtOAc (3×25 mL). The combined organic layers were washed with brine (1×100 mL), dried over anhydrous Na₂SO₄.

After filtration, the filtrate was concentrated under reduced pressure. The residue was purified by silica gel column chromatography, eluted with PE/EtOAc (12:1) to afford tert-butyl (2S)-2-((4-chloro-N-(2-ethoxy-2-oxoethyl)-2-nitrophenyl) sulfonamido)-3-(6-fluoro-2,3-dimethylphenyl) butanoate (1.33 g, 63.05%).

### Step 5: Synthesis of tert-butyl (2S)-2-((2-amino-4-chloro-N-(2-ethoxy-2-oxoethyl) phenyl) sulfonamido)-3-(6-fluoro-2,3-dimethylphenyl) butanoate

To a stirred solution of methyl (tert-butyl (2S)-2-((4-chloro-N-(2-ethoxy-2-oxoethyl)-2-nitrophenyl) sulfonamido)-3-(6-fluoro-2,3-dimethylphenyl) butanoate (970 mg, 2.1 mmol, 1 equiv) in AcOH (15 mL) was added Fe (1.24 g, 22.1 mmol, 10 equiv) in portions at room temperature. The resulting mixture was stirred for 15 min at 70° C. The mixture was allowed to cool down to room temperature. The resulting mixture was filtered, the filter cake was washed with ethyl acetate (3×30 mL). The filtrate was concentrated under reduced pressure. The residue was purified by reverse flash chromatography with the following conditions: column, C18 silica gel; mobile phase, MeCN in Water (0.1% FA), 10% to 100% gradient in 20 min; detector, UV 254 nm. This resulted in tert-butyl (2S)-2-((2-amino-4-chloro-N-(2-ethoxy-2-oxoethyl) phenyl) sulfonamido)-3-(6-fluoro-2,3-dimethylphenyl)butanoate (987 mg, 80.01%).

### Step 6: Synthesis of N-((2-amino-4-chlorophenyl)sulfonyl)-N-((2S)-1-(tert-butoxy)-3-(6-fluoro-2,3-dimethylphenyl)-1-oxobutan-2-yl)glycine

To a stirred solution of tert-butyl (2S)-2-((2-amino-4-chloro-N-(2-ethoxy-2-oxoethyl) phenyl) sulfonamido)-3-(6-fluoro-2,3-dimethylphenyl) butanoate(950 mg, 1.71 mmol, 1 equiv) in THF and H₂O (9.5 mL) was added lithium hydroxide (358 mg, 8.5 mmol, 5 equiv) in portions at room temperature. The resulting mixture was stirred for overnight at 65° C. The mixture was acidified to pH 5 with HCl (2M). The resulting mixture was extracted with EtOAc (3×25 mL). The combined organic layers were washed with brine (1×50 mL), dried over anhydrous Na₂SO₄. After filtration, the filtrate was concentrated under reduced pressure. The crude product resulting mixture was used in the next step directly without further purification.

### Step 7: Synthesis of tert-butyl (2S)-2-(7-chloro-1,1-dioxido-4-oxo-4,5-dihydrobenzo[f][1,2,5]thiadiazepin-2 (3H)-yl)-3-(6-fluoro-2,3-dimethylphenyl)butanoate

To a stirred solution of N-((2-amino-4-chlorophenyl) sulfonyl)-N-((2S)-1-(tert-butoxy)-3-(6-fluoro-2,3-dimethylphenyl)-1-oxobutan-2-yl) glycine (800 mg, 1.51 mmol, 1 equiv) in DCM was added EDCI (319 mg, 1.66 mmol, 1.1 equiv) in portions at room temperature. To the above mixture was added DMAP (18.5 mg, 0.15 mmol, 0.1 equiv) at room temperature. The resulting mixture was stirred for additional 2h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in tert-butyl (2S)-2-(7-chloro-1,1-dioxido-4-oxo-4,5-dihydrobenzo[f][1,2,5]thiadiazepin-2 (3H)-yl)-3-(6-fluoro-2,3-dimethylphenyl)butanoate (430 mg, 55.6%).

### Step 8: Synthesis of tert-butyl (2S)-2-(7-chloro-1,1-dioxido-4,5-dihydrobenzo[f][1,2,5]thiadiazepin-2 (3H)-yl)-3-(6-fluoro-2,3-dimethylphenyl)butanoate

To a stirred solution of tert-butyl (2S)-2-(7-chloro-1,1-dioxido-4-oxo-4,5-dihydrobenzo[f][1,2,5]thiadiazepin-2 (3H)-yl)-3-(6-fluoro-2,3-dimethylphenyl)butanoate (500 mg, 0.95 mmol, 1 equiv) in THF (5 mL) was added BH₃-THF (5 mL, 5 mmol, 5.25 equiv) dropwise at room temperature. The resulting mixture was stirred for 4 h at room temperature under nitrogen atmosphere. The reaction was quenched with MeOH at room temperature. The resulting mixture was concentrated under vacuum. The crude product was used in the next step directly without further purification.

### Step 9: Synthesis of (2S)-2-(7-chloro-1,1-dioxido-4,5-dihydrobenzo[f][1,2,5]thiadiazepin-2 (3H)-yl)-3-(6-fluoro-2,3-dimethylphenyl)butanoic acid

To a stirred solution of tert-butyl (2S)-2-(7-chloro-1,1-dioxido-4,5-dihydrobenzo[f][1,2,5]thiadiazepin-2 (3H)-yl)-3-(6-fluoro-2,3-dimethylphenyl)butanoate (280 mg, 0.55 mmol, 1 equiv) in DCM (5 mL) was added TFA (5 mL) at room temperature. The resulting mixture was stirred overnight at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in 2S)-2-(7-chloro-1,1-dioxido-4,5-dihydrobenzo[f][1,2,5]thiadiazepin-2 (3H)-yl)-3-(6-fluoro-2,3-dimethylphenyl)butanoic acid (255 mg, 57.3%).

### Step 10: Synthesis of 5-((1S,2R)-1-(7-chloro-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

To a stirred solution of 2S)-2-(7-chloro-1,1-dioxido-4,5-dihydrobenzo[f][1,2,5]thiadiazepin-2 (3H)-yl)-3-(6-fluoro-2,3-dimethylphenyl)butanoic acid (235 mg, 0.53 mmol, 1 equiv) in THF (2.35 mL) was added CDI (216 mg, 1.33 mmol, 2.5 equiv) in portions at room temperature. The resulting mixture was stirred 30 min at room temperature. To the above mixture was added hydrazine hydrate (129.5 μL, 2.67 mmol, 5 equiv) dropwise at 0° C. The resulting mixture was stirred for additional 30 min at 0° C. The reaction was quenched with Water/Ice at 0° C. The resulting mixture was extracted with EtOAc (2×20 mL).

The combined organic layers were washed with brine (1×30 mL), dried over anhydrous Na₂SO₄. After filtration, the filtrate was concentrated under reduced pressure. The residue was dissolved in 1,4-dioxane (2.0 mL). To the above mixture was added CDI (259 mg, 1.6 mmol, 3 equiv) in portions at room temperature. The resulting mixture was stirred for additional 1 h at room temperature. The reaction was quenched with water at room temperature. The resulting mixture was concentrated under vacuum.

The crude product (260 mg) was purified by Chiral-Prep-HPLC with the following conditions: Column, XBridge Shield RP18 OBD Column, 30*150 mm, 5 μm; mobile phase, Water(10 mmol/L NH₄HCO₃+0.1% NH₃·H₂O) and ACN (26% ACN up to 56% in 8 min); Detector, UV 254 nm. This resulted in 5-((1S,2R)-1-(7-chloro-1,1-dioxido-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (152 mg, 58.4%). LCMS: ES, m/z)(M−H)=478.9. ¹H NMR (300 MHz, Methanol-d₄) δ 7.58 (d, J=8.6 Hz, 1H), 7.00-6.95 (dd, J=8.4, 5.7 Hz, 1H), 6.79-6.67 (m, 3H), 5.46-5.42 (dd, J=11.7, 1.7 Hz, 1H), 4.06-3.96 (ddd, J=14.9, 11.0, 4.2 Hz, 1H), 3.82-3.72 (ddd, J=16.7, 9.5, 5.6 Hz, 2H), 3.62-3.55 (ddd, J=13.0, 4.2, 2.2 Hz, 1H), 3.35 (dd, J=5.7, 2.2 Hz, 4H), 2.34 (s, 3H), 2.21 (s, 3H), 1.44 (dd, J=7.0, 1.1 Hz, 3H).

### Example 22: 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxamide 1,1-dioxide

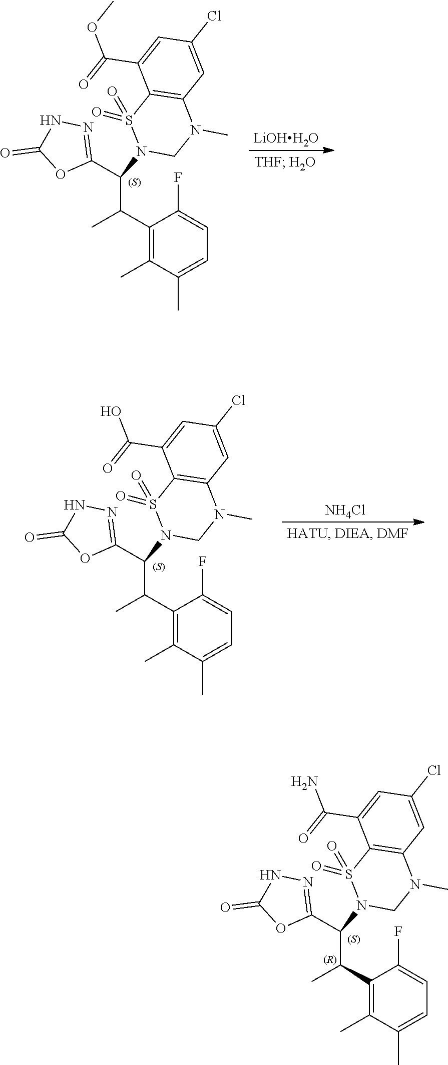

### Step 1: Synthesis of 6-chloro-2-((1S)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylic acid 1,1-dioxide

To a stirred solution of methyl 6-chloro-2-((1S)-2-(6-fluoro-2,3-dimethylphenyl)-1-(5-oxo-4,5-dihydro-1,3,4-oxadiazol-2-yl)propyl)-4-methyl-3,4-dihydro-2H-benzo[e][1,2,4]thiadiazine-8-carboxylate 1,1-dioxide (from example 19 (100 mg, 0.19 mmol, 1 equiv) in THF (1.5 mL) were added LiOH·H₂O (15.6 mg, 0.37 mmol, 2 equiv) and H₂O (0.5 mL) dropwise at room temperature. The resulting mixture was stirred overnight at 65° C. The mixture was acidified to pH 5 with citric acid. The resulting mixture was extracted with EtOAc (1×20 mL). The combined organic layers were washed with brine (1×10 mL), dried over anhydrous Na₂SO₄. After filtration, the filtrate was concentrated under reduced pressure. The crude product was used in the next step directly without further purification.

### Step 2: Synthesis of 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxamide 1,1-dioxide

To a stirred solution of 6-chloro-2-((1S)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxylic acid 1,1-dioxide (95 mg, 0.18 mmol, 1 equiv) in DMF (1 mL) were added DIEA (95 μL, 0.54 mmol, 3 equiv), HATU (103 mg, 0.27 mmol, 1.5 equiv) and ammonium chloride (1.53 mg, 0.03 mmol, 1.5 equiv) at room temperature. The resulting mixture was stirred 1 h at room temperature. The resulting mixture was extracted with EtOAc). The combined organic layers were washed with brine, dried over anhydrous Na₂SO₄. After filtration, the filtrate was concentrated under reduced pressure. The crude product (90 mg) was purified by Prep-HPLC with the following conditions (Column: XSelect CSH Prep C18 OBD Column, 19*250 mm, 5Î¼ rm; Mobile Phase A: Water(0.1% FA), Mobile Phase B: MeOH-HPLC; Flow rate: 20 mL/min; Gradient: 20% B to 50% B in 8 min, 50% B; Wave Length: 254 nm; RT1 (min): 7.45) to afford 6-chloro-2-((1S,2R)-2-(6-fluoro-2,3-dimethylphenyl)-1-(5-oxo-4,5-dihydro-1,3,4-oxadiazol-2-yl) propyl)-4-methyl-3,4-dihydro-2H-benzo[e][1,2,4]thiadiazine-8-carboxamide 1,1-dioxide (12.2 mg, 13%). LC-MS: (ES, m/z): [M−H]⁺=522.05. ¹H NMR (300 MHz, Methanol-d₄) δ 7.01-6.96 (dd, J=8.4, 5.7 Hz, 1H), 6.82-6.67 (m, 3H), 5.47-5.39 (m, 2H), 5.07-5.02 (d, J=14.6 Hz, 1H), 3.94-3.83 (dd, J=12.1, 7.2 Hz, 1H), 2.91 (s, 3H), 2.38 (s, 3H), 2.22 (s, 3H), 1.44 (dd, J=7.0, 1.1 Hz, 3H).

### Example 23: 7-chloro-2-((1S,2R)-2-(6-fluoro-2,3-dimethylphenyl)-1-(5-oxo-4,5-dihydro-1,3,4-oxadiazol-2-yl) propyl)-3,4-dihydro-2H-benzo[b][1,4,5]oxathiazepine-9-carboxamide 1,1-dioxide

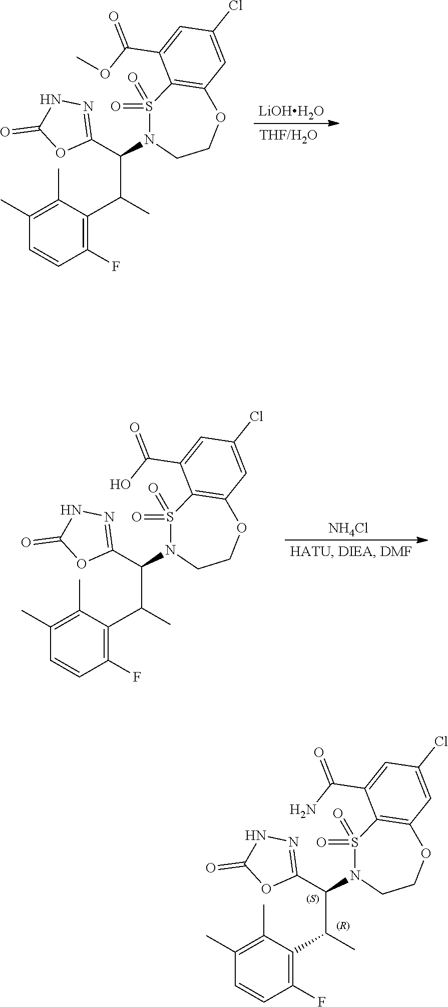

### Step 1: Synthesis of 7-chloro-2-((1S)-2-(6-fluoro-2,3-dimethylphenyl)-1-(5-oxo-4,5-dihydro-1,3,4-oxadiazol-2-yl)propyl)-3,4-dihydro-2H-benzo[b][1,4,5]oxathiazepine-9-carboxylic acid 1,1-dioxide

To a stirred solution of methyl 7-chloro-2-[(1S)-2-(6-fluoro-2,3-dimethylphenyl)-1-(5-oxo-4H-1,3,4-oxadiazol-2-yl)propyl]-1,1-dioxo-3,4-dihydro-5,1lambda6,2-benzoxathiazepine-9-carboxylate (from example 18) (80 mg, 0.15 mmol, 1 equiv) and lithiumol hydrate (31 mg, 0.74 mmol, 5 equiv) in THF was added H₂O (200 μL) at room temperature. The resulting mixture was stirred overnight at 60° C. The mixture was acidified to pH 6 with AcOH. The resulting mixture was extracted with EtOAc (3×60 mL). The combined organic layers were washed with brine (2×100 mL), dried over anhydrous Na₂SO₄. After filtration, the filtrate was concentrated under reduced pressure. The crude product was used in the next step directly without further purification.

### Step 2: Synthesis of 7-chloro-2-((1S,2R)-2-(6-fluoro-2,3-dimethylphenyl)-1-(5-oxo-4,5-dihydro-1,3,4-oxadiazol-2-yl)propyl)-3,4-dihydro-2H-benzo[b][1,4,5]oxathiazepine-9-carboxamide 1,1-dioxide

To a stirred solution of 7-chloro-2-((1S)-2-(6-fluoro-2,3-dimethylphenyl)-1-(5-oxo-4,5-dihydro-1,3,4-oxadiazol-2-yl)propyl)-3,4-dihydro-2H-benzo[b][1,4,5]oxathiazepine-9-carboxylic acid 1,1-dioxide (90 mg, 0.17 mmol, 1 equiv) and HATU (100 mg, 0.26 mmol, 1.5 equiv) in DMF was added ammonium chloride (11 mg, 0.21 mmol, 1.2 equiv) in portions at room temperature. The resulting mixture was stirred for 30 min at room temperature. The residue was purified by reverse flash chromatography with the following conditions:Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 23% B to 50% B in 8 min, 50% B; Wave Length: 254 nm; RT1 (min): 7. his resulted in 7-chloro-2-((1S,2R)-2-(6-fluoro-2,3-dimethylphenyl)-1-(5-oxo-4,5-dihydro-1,3,4-oxadiazol-2-yl)propyl)-3,4-dihydro-2H-benzo[b][1,4,5]oxathiazepine-9-carboxamide 1,1-dioxide (23.4 mg, 26%). LC-MS: (ES, m/z): [M−H]=523.10. ¹H NMR (300 MHz, Methanol-d₄) δ 7.25-7.24 (d, J=2.1 Hz, 1H), 7.08-7.07 (d, J=2.2 Hz, 1H), 6.98-6.95 (dd, J=8.4, 5.7 Hz, 1H), 6.72-6.68 (dd, J=12.0, 8.4 Hz, 1H), 5.55-5.51 (dd, J=11.6, 1.8 Hz, 1H), 4.85-4.81 (s, 1H), 4.40-4.50 (d, J=12.0 Hz, 1H), 4.01-3.98 (ddd, J=15.9, 10.6, 5.7 Hz, 1H), 3.81-3.76 (t, J=9.8 Hz, 2H), 2.35-2.40 (s, 3H), 2.39-2.21 (s, 3H), 1.44-1.42 (dd, J=6.9, 1.1 Hz, 3H).

### Example 24: 5-((1S,2R)-1-(7-chloro-9-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

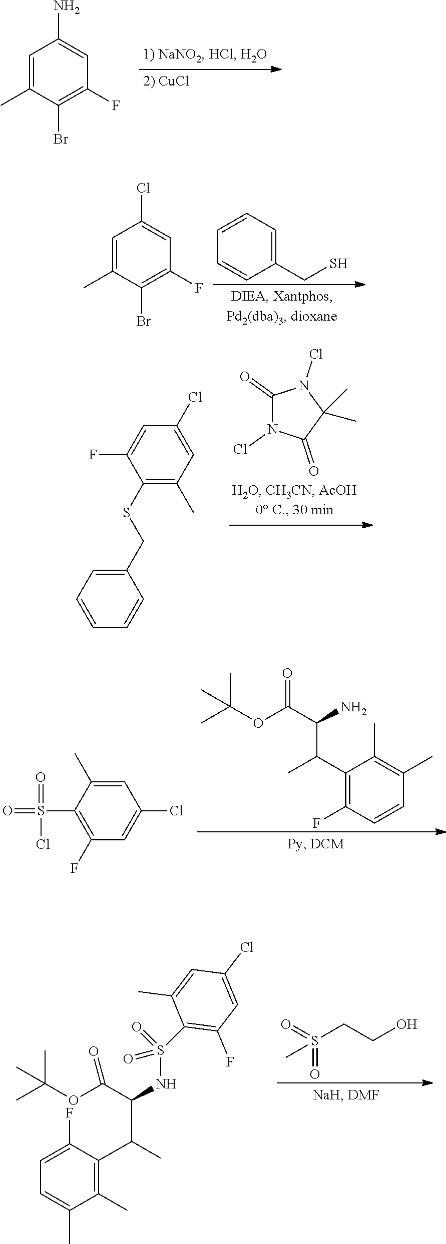

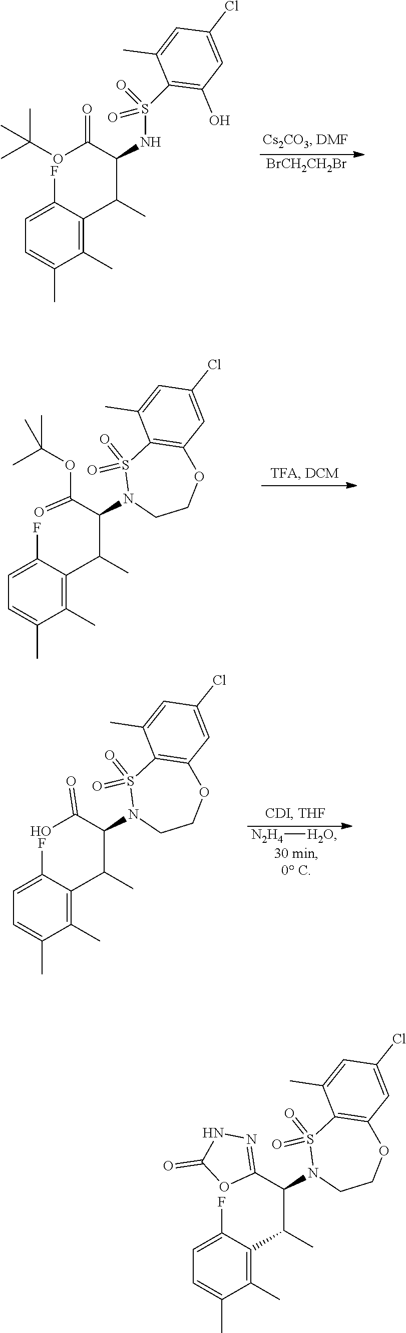

### Step 1: Synthesis of 2-bromo-5-chloro-1-fluoro-3-methylbenzene

Into a 40 mL vial were added 4-bromo-3-fluoro-5-methylaniline (3.65 g, 17.9 mmol, 1 equiv) and HC (36.5 mL, 1201 mmol, 67 equiv) at room temperature. To the above mixture was added NaNO₂ (2468 mg, 35.86 mmol, 2 equiv) in H₂O (0.6 mL) dropwise at 0° C. The resulting mixture was stirred for additional 30 min at room temperature. To the above mixture was added CuCl (5313 mg, 53.6 mmol, 3 equiv) in portions at room temperature. The resulting mixture was stirred for additional 1 h at 60° C. The mixture was allowed to cool down to room temperature. The reaction was quenched with water at room temperature. The resulting mixture was extracted with DCM (2×15 mL). The combined organic layers were washed with brine (1×20 mL), dried over anhydrous MgSO₄. After filtration, the filtrate was concentrated under reduced pressure. The residue was purified by Prep-TLC (PE/EtOAc 5:1) to afford 2-bromo-5-chloro-1-fluoro-3-methylbenzene (3.2 g, 80.1%).

### Step 2: Synthesis of benzyl(4-chloro-2-fluoro-6-methylphenyl) sulfane

To a stirred solution of 2-bromo-5-chloro-1-fluoro-3-methylbenzene (2 g, 8.95 mmol, 1 equiv) and DIEA (4676 μL, 26.9 mmol, 3 equiv) in dioxane was added Xantphos (1036 mg, 1.79 mmol, 0.2 equiv) Pd₂(dba)₃ (820 mg, 0.9 mmol, 0.1 equiv) at room temperature. The resulting mixture was stirred overnight at 100° C. under nitrogen atmosphere. The reaction was quenched with water at room temperature.

The resulting mixture was extracted with EtOAc (2×50 mL). The combined organic layers were washed with brine (1×100 mL), dried over anhydrous Na₂SO₄. After filtration, the filtrate was concentrated under reduced pressure. The residue was purified by Prep-TLC (PE/EtOAc 10:1) to afford benzyl(4-chloro-2-fluoro-6-methylphenyl) sulfane (1.45 g, 60.7%).

### Step 3: Synthesis of 4-chloro-2-fluoro-6-methylbenzenesulfonyl chloride

To a stirred solution of benzyl(4-chloro-2-fluoro-6-methylphenyl) sulfane (1.45 g, 5.44 mmol, 1 equiv) and AcOH (1557 μL, 27 mmol, 5 equiv) H₂O (881 μL) in MeCN was added 1,3-dichloro-5,5-dimethylimidazolidine-2,4-dione (2142 mg, 10.9 mmol, 2 equiv) in portions at 0° C. The resulting mixture was stirred for 30 min at 0° C. The reaction was quenched with water at room temperature. The resulting mixture was extracted with EtOAc (3×50 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (12:1) to afford 4-chloro-2-fluoro-6-methylbenzenesulfonyl chloride (930 mg, 70.4%).

### Step 4: Synthesis of tert-butyl (2S)-2-((4-chloro-2-fluoro-6-methylphenyl)sulfonamido)-3-(6-fluoro-2,3-dimethylphenyl)butanoate

To a stirred mixture of tert-butyl (2S)-2-amino-3-(6-fluoro-2,3-dimethylphenyl) butanoate (1100 mg, 3.91 mmol, 1 equiv) and pyridine (15811 μL, 19.5 mmol, 5 equiv) in DCM was added 4-chloro-2-fluoro-6-methylbenzenesulfonyl chloride (950 mg, 3.909 mmol, 1 equiv) dropwise at 0° C. The resulting mixture was stirred overnight at room temperature. The resulting mixture was concentrated under vacuum. The residue was purified by silica gel column chromatography, eluted with PE/EA (8:1) to afford tert-butyl (2S)-2-((4-chloro-2-fluoro-6-methylphenyl) sulfonamido)-3-(6-fluoro-2,3-dimethylphenyl) butanoate (1.33 g, 69.7%).

### Step 5: Synthesis of tert-butyl (2S)-2-((4-chloro-2-hydroxy-6-methylphenyl) sulfonamido)-3-(6-fluoro-2,3-dimethylphenyl) butanoate

To a stirred solution of tert-butyl (2S)-2-((4-chloro-2-fluoro-6-methylphenyl) sulfonamido)-3-(6-fluoro-2,3-dimethylphenyl) butanoate (300 mg, 0.62 mmol, 1 equiv) and 2-methanesulfonylethanol (229 mg, 1.85 mmol, 3 equiv) in DMF (3 mL) was added NaH (123 mg, 3.08 mmol, 5 equiv, 60%) in portions at 0° C. The resulting mixture was stirred for 2 days at 60° C. The reaction was quenched with Water at room temperature. The resulting mixture was extracted with EtOAc (2×5 mL). The combined organic layers were washed with brine (1×10 mL), dried over anhydrous Na₂SO₄. After filtration, the filtrate was concentrated under reduced pressure. The residue was purified by Prep-TLC (PE/EA 3:1) to afford tert-butyl (2S)-2-((4-chloro-2-hydroxy-6-methylphenyl) sulfonamido)-3-(6-fluoro-2,3-dimethylphenyl) butanoate (177 mg, 59.2%).

### Step 6: Synthesis of tert-butyl (2S)-2-(7-chloro-9-methyl-1,1-dioxido-3,4-dihydro-2H-benzo[b][1,4,5]oxathiazepin-2-yl)-3-(6-fluoro-2,3-dimethylphenyl)butanoate

To a stirred solution of tert-butyl (2S)-2-((4-chloro-2-hydroxy-6-methylphenyl) sulfonamido)-3-(6-fluoro-2,3-dimethylphenyl) butanoate (100 mg, 0.21 mmol, 1 equiv) in DMF (1 mL) were added Cs₂CO₃ (201 mg, 0.62 mmol, 3 equiv) and dibromoethane (17.7 μL, 0.21 mmol, 1 equiv) at room temperature. The resulting mixture was stirred for overnight at 60° C. The residue was purified by reverse flash chromatography with the following conditions: column, C18 silica gel; mobile phase, MeCN in Water (10 mmol/L NH₄HCO₃), 10% to 100% gradient in 10 min; detector, UV 254 nm. This resulted in tert-butyl (2S)-2-(7-chloro-9-methyl-1,1-dioxido-3,4-dihydro-2H-benzo[b][1,4,5]oxathiazepin-2-yl)-3-(6-fluoro-2,3-dimethylphenyl)butanoate (70 mg, 39.08%).

### Step 7: Synthesis of (2S)-2-(7-chloro-9-methyl-1,1-dioxido-3,4-dihydro-2H-benzo[b][1,4,5]oxathiazepin-2-yl)-3-(6-fluoro-2,3-dimethylphenyl)butanoic acid

To a stirred solution/mixture tert-butyl (2S)-2-(7-chloro-9-methyl-1,1-dioxido-3,4-dihydro-2H-benzo[b][1,4,5] oxathiazepin-2-yl)-3-(6-fluoro-2,3-dimethylphenyl)butanoate (70 mg, 0.14 mmol, 1 equiv) in DCM (1 mL) was added TFA (500 μL) at room temperature. The resulting mixture was stirred 3 h at room temperature. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in (2S)-2-(7-chloro-9-methyl-1,1-dioxido-3,4-dihydro-2H-benzo[b][1,4,5]oxathiazepin-2-yl)-3-(6-fluoro-2,3-dimethylphenyl)butanoic acid (60 mg, 96.3%).

Ste 8: Synthesis of 5-((1S,2R)-1-(7-chloro-9-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 8 mL vial were added (2S)-2-(7-chloro-9-methyl-1,1-dioxido-3,4-dihydro-2H-benzo[b][1,4,5]oxathiazepin-2-yl)-3-(6-fluoro-2,3-dimethylphenyl)butanoic acid (10 mg, 0.022 mmol, 1 equiv) in THF (100 μL). To the above mixture was added CDI (5.3 mg, 0.03 mmol, 1.5 equiv) in portions at room temperature. The resulting mixture was stirred for additional 30 min at room temperature. To the above mixture was added N₂H₄·H₂O (3.20 μL, 0.066 mmol, 3 equiv) dropwise at 0° C.

The resulting mixture was stirred for additional 30 min at 0° C. The reaction was quenched with water at 0° C. The resulting mixture was extracted with EtOAc (2×1 mL). The combined organic layers were washed with brine (1×2 mL), dried over anhydrous Na₂SO₄. After filtration, the filtrate was concentrated under reduced pressure to afford crude product SM1. Into an 8 mL vial were added SM1 in dioxane (100 μL). To the above mixture was added CDI (8.9 mg, 0.055 mmol, 2.5 equiv) in portions at room temperature.

The resulting mixture was stirred for additional 30 min at room temperature. The crude product (60 mg) was purified by Prep-HPLC with the following conditions (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 20% B to 50% B in 8 min, 50% B; Wave Length: 254 nm; RTI(min): 7;) to afford 5-((1S,2R)-1-(7-chloro-9-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (12.3 mg, 20.21%). LCMS:(ES, m/z): [M−H]⁺=494.1. ¹H NMR (300 MHz, Methanol-d₄) δ 7.12-7.08 (m, 1H), 6.98-6.96 (dd, J=8.4, 5.8 Hz, 1H), 6.86-6.85 (d, J=2.2 Hz, 1H), 6.75-6.72 (dd, J=12.1, 8.4 Hz, 1H), 5.63-5.59 (dd, J=11.7, 1.9 Hz, 1H), 4.46-4.37 (ddt, J=16.5, 12.0, 5.3 Hz, 2H), 4.01-3.91 (ddd, J=14.1, 10.6, 6.4 Hz, 1H), 3.85-3.71 (m, 2H), 2.65 (s, 3H), 2.36 (s, 3H), 2.22 (s, 3H), 1.44 (dd, J=7.0, 1.1 Hz, 3H).

### Example 25: 5-((1S,2R)-1-(6-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

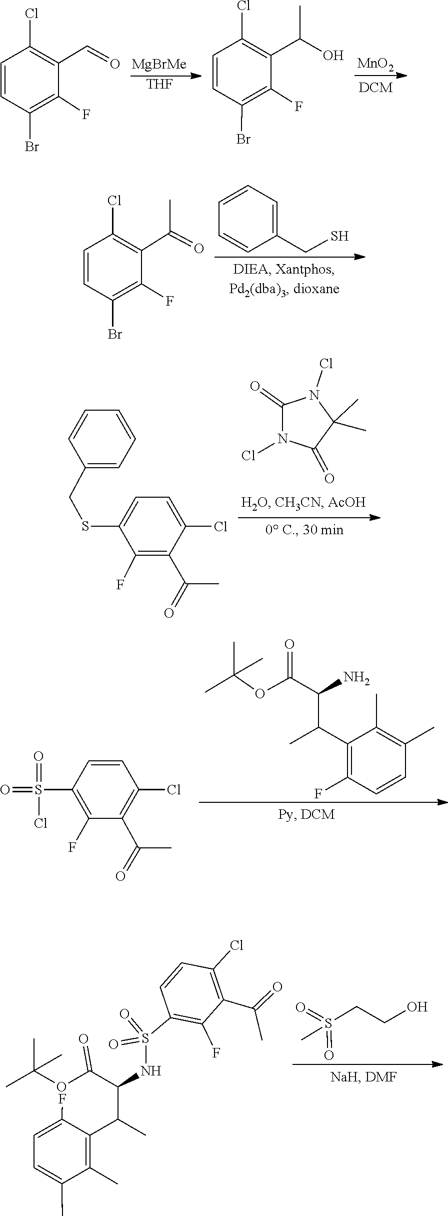

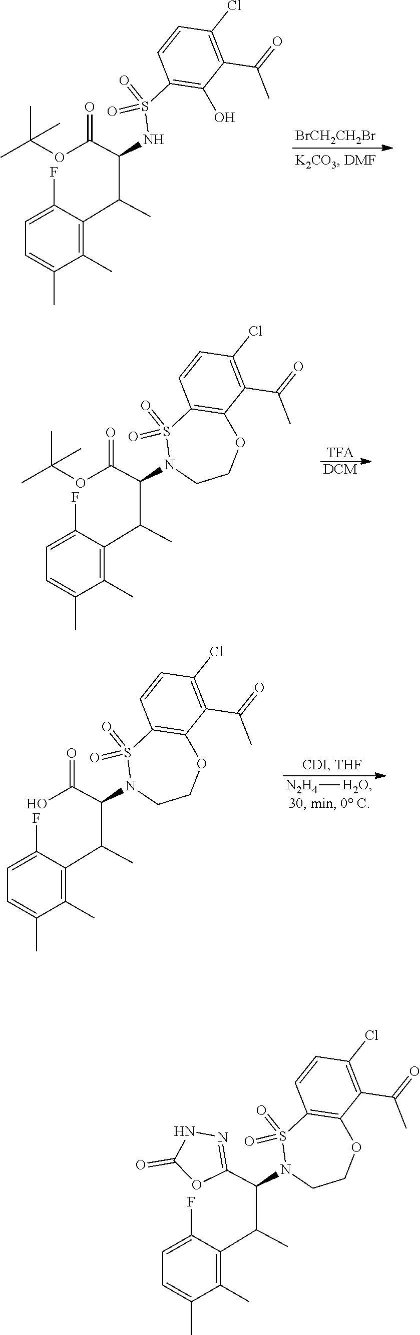

### Step 1: Synthesis of 1-(3-bromo-6-chloro-2-fluorophenyl) ethan-1-ol

To a stirred solution of 3-bromo-6-chloro-2-fluorobenzaldehyde (5 g, 21 mmol, 1 equiv) in THF was added CH₃MgBr (3M in Et₂O) (17.6 mL, 52.6 mmol, 2.5 equiv) dropwise at 0° C. under nitrogen atmosphere. The resulting mixture was stirred 30 min at room temperature under nitrogen atmosphere. The reaction was quenched with sat. NH₄Cl (aq.) at room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The crude product was used in the next step directly without further purification.

### Step 2: Synthesis of 1-(3-bromo-6-chloro-2-fluorophenyl) ethan-1-one

Into a 250 mL round-bottom flask were added 1-(3-bromo-6-chloro-2-fluorophenyl) ethanol (5.1 g, 20 mmol, 1 equiv), DCM (100 mL) and MnO₂ (17.5 g, 201 mmol, 10 equiv) at room temperature. The resulting mixture was stirred overnight at 40° C. The resulting mixture was filtered, the filter cake was washed with CH₂Cl₂ (3×50 mL). The filtrate was concentrated under reduced pressure. The residue was purified by silica gel column chromatography, eluted with PE to afford 1-(3-bromo-6-chloro-2-fluorophenyl) ethanone (3.95 g, 78%) as a light-yellow oil.

### Step 3: Synthesis of 1-(3-(benzylthio)-6-chloro-2-fluorophenyl) ethan-1-one

Into a 100 mL round-bottom flask were added 1-(3-bromo-6-chloro-2-fluorophenyl) ethanone (2 g, 7.95 mmol, 1 equiv), dioxane (35 mL), benzyl mercaptan (1.13 mL, 9.5 mmol, 1.2 equiv), DIEA (4.16 mL, 23.8 mmol, 3 equiv), Xantphos (920 mg, 1.59 mmol, 0.2 equiv) and Pd₂(dba)₃ (728 mg, 0.8 mmol, 0.1 equiv) at room temperature. The resulting mixture was stirred overnight at 110° C. under nitrogen atmosphere. The resulting mixture was filtered, the filter cake was washed with DCM (3×10 mL). The filtrate was concentrated under reduced pressure. The residue was purified by silica gel column chromatography, eluted with PE/EtOAc (10:1) to afford 1-(3-(benzylthio)-6-chloro-2-fluorophenyl) ethan-1-one (2.44 g, 93.7%) as a light-yellow solid.

### Step 4: Synthesis of 3-acetyl-4-chloro-2-fluorobenzenesulfonyl chloride

To a stirred solution of 1-(3-(benzylthio)-6-chloro-2-fluorophenyl) ethan-1-one (2.44 g, 8.28 mmol, 1 equiv), H₂O (0.8 mL), AcOH (4 mL) in MeCN (20 mL) was added 1,3-dichloro-5,5-dimethylimidazolidine-2,4-dione (3.26 g, 16.56 mmol, 2 equiv) dropwise at 0° C. The resulting mixture was stirred for 30 min at 0° C. The reaction was quenched with sat. NH₄C₁ (aq.) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (8:1) to afford 3-acetyl-4-chloro-2-fluorobenzenesulfonyl chloride (1.8 g, 80.2%).

### Step 5: Synthesis of tert-butyl (2S)-2-((3-acetyl-4-chloro-2-fluorophenyl) sulfonamido)-3-(6-fluoro-2,3-dimethylphenyl) butanoate

Into a 100 mL round-bottom flask were added tert-butyl (2S)-2-amino-3-(6-fluoro-2,3-dimethylphenyl) butanoate (1.70 g, 6.04 mmol, 1 equiv), DCM (15 mL) and Pyridine (2.4 mL, 30 mmol, 5 equiv) at 0° C. To the above mixture was added 3-acetyl-4-chloro-2-fluorobenzenesulfonyl chloride (1.8 g, 6.6 mmol, 1.1 equiv) in DCM (15 mL) at 0° C. The resulting mixture was stirred overnight at room temperature. The resulting mixture was washed with 1×20 mL of water. The resulting mixture was extracted with DCM (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (7:1) to afford of tert-butyl (2S)-2-((3-acetyl-4-chloro-2-fluorophenyl) sulfonamido)-3-(6-fluoro-2,3-dimethylphenyl) butanoate (2.58 g, 82.8%).

### Step 6: Synthesis of tert-butyl (2S)-2-((3-acetyl-4-chloro-2-hydroxyphenyl) sulfonamido)-3-(6-fluoro-2,3-dimethylphenyl) butanoate

To a solution of tert-butyl (2S)-2-((3-acetyl-4-chloro-2-fluorophenyl) sulfonamido)-3-(6-fluoro-2,3-dimethylphenyl) butanoate (1 g, 1.94 mmol, 1 equiv) in DMF was added sodium hydride (60% in oil, 310 mg) at 0° C. The mixture was stirred for 15 min. 2-methanesulfonylethanol (481 mg, 3.88 mmol, 2 equiv) was added and the mixture was allowed to warm to RT and stirred 2 h at room temperature under nitrogen atmosphere. The reaction was quenched with sat. NH₄Cl (aq.) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (1:1) to afford of tert-butyl (2S)-2-((3-acetyl-4-chloro-2-hydroxyphenyl) sulfonamido)-3-(6-fluoro-2,3-dimethylphenyl) butanoate (410 mg, 41.2%) as a brown semi-solid.

### Step 7: Synthesis of tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate

Into a 40 mL sealed tube were added tert-butyl (2S)-2-((3-acetyl-4-chloro-2-hydroxyphenyl) sulfonamido)-3-(6-fluoro-2,3-dimethylphenyl) butanoate (400 mg, 0.78 mmol, 1 equiv), DMF (8 mL), dibromoethane (100 μL, 1.17 mmol, 1.5 equiv) and K₂CO₃ (323 mg, 2.33 mmol, 3 equiv) at room temperature. The resulting mixture was stirred overnight at 65° C. The reaction was quenched with sat.

NH₄C₁ (aq.) at room temperature. The resulting mixture was extracted with EtOAc (3×10 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (10:1) to afford tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate (300 mg, 71.4%).

### Step 8: Synthesis of (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoic acid

Into a 8 mL sealed tube were added afford tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate (300 mg, 0.56 mmol, 1 equiv), DCM (1.5 mL) and TFA (1.5 mL) at room temperature. The resulting mixture was stirred for 1 h at room temperature. The resulting mixture was concentrated under vacuum. The crude product was used in the next step directly without further purification.

### Step 9: Synthesis of 5-((1S,2R)-1-(6-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 8 mL sealed tube were added of(2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoic acid (55 mg, 0.11 mmol, 1 equiv), THF (1 mL) and CDI (46.1 mg, 0.29 mmol, 2.5 equiv) at room temperature. The resulting mixture was stirred 40 min at room temperature. To the above mixture was added hydrazine hydrate (16.6 μL, 0.34 mmol, 3 equiv) dropwise at 0° C. The resulting mixture was stirred for additional 30 min at 0° C. The reaction was quenched with sat. NH₄Cl (aq.) at room temperature. The resulting mixture was extracted with EtOAc (3×1 mL), dried over anhydrous Na₂SO₄. After filtration, the filtrate was concentrated under reduced pressure. To the above mixture was added dioxane (1 mL) and CDI (46.1 mg, 0.29 mmol, 2.5 equiv) at room temperature. The resulting mixture was stirred for 30 min at room temperature. The reaction was quenched with sat. NH₄Cl (aq.) at room temperature. The resulting mixture was extracted with EtOAc (3×5 mL). The combined organic layers were washed with brine (3×5 mL), dried over anhydrous Na₂SO₄ and evaporated. The crude product was purified by Chiral-Prep-HPLC with the following conditions: Column: XBridge Prep OBD C18 Column, 19*250 mm, 5 μm; Mobile Phase A: Water(10 mmol/L NH₄HCO₃+0.1% NH₃·H₂O), Mobile Phase B: MeOH-HPLC; Flow rate: 20 mL/min; Gradient: 20% B to 50% B in 8 min, 50% B; Wave Length: 254 nm; RT1 (min): 7.45 to afford of 5-((1S,2R)-1-(6-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (14.6 mg, 23.6%).

LCMS: (ES, m/z):(M−H):522.10. ¹H NMR (300 MHz, Methanol-d₄) δ 7.82-7.79 (d, J=8.6 Hz, 1H), 7.36-7.33 (d, J=8.5 Hz, 1H), 6.99-6.97 (dd, J=8.4, 5.7 Hz, 1H), 6.76-6.69 (dd, J=12.1, 8.4 Hz, 1H), 5.55-5.51 (dd, J=11.6, 2.2 Hz, 1H), 4.62 (dt, J=12.2, 4.6 Hz, 1H), 3.96-3.93 (m, 4H), 2.44 (s, 3H), 2.33 (s, 3H), 2.21-2.03 (s, 3H), 1.45-1.42 (dd, J=7.0, 1.2 Hz, 3H).

### Example 26: 5-((1S,2R)-1-(6-chloro-4,8-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

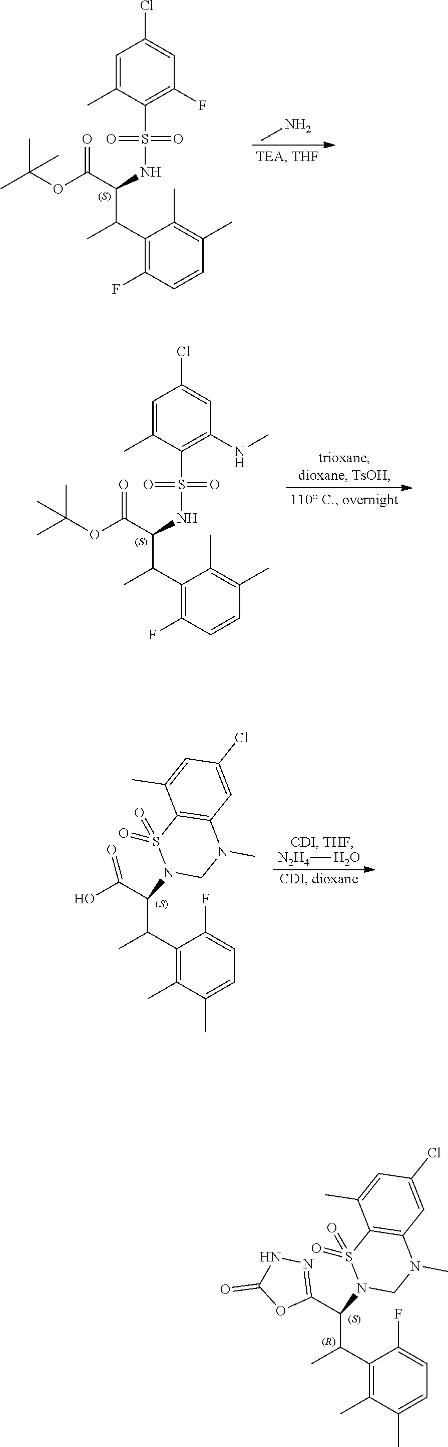

### Step 1: Synthesis of tert-butyl (2S)-2-((4-chloro-2-methyl-6-(methylamino) phenyl) sulfonamide)-3-(6-fluoro-2,3-dimethylphenyl) butanoate

To a stirred solution of tert-butyl (2S)-2-(4-chloro-2-fluoro-6-methylbenzenesulfonamido)-3-(6-fluoro-2,3-dimethylphenyl) butanoate (see example 24) (300 mg, 0.62 mmol, 1 equiv) in THF (3 mL) were added TEA (855 μL, 6.15 mmol, 10 equiv) and methylamine (1.54 mL, 3.1 mmol, 5 equiv) at room temperature. The resulting mixture was stirred overnight at 60° C. The resulting mixture was concentrated under vacuum. The residue was purified by silica gel column chromatography, eluted with PE/EtOAc (9:1) to afford tert-butyl (2S)-2-((4-chloro-2-methyl-6-(methylamino) phenyl) sulfonamide)-3-(6-fluoro-2,3-dimethylphenyl) butanoate (285 mg, 92.9%).

### Step 2: Synthesis of (2S)-2-(6-chloro-4,8-dimethyl-1,1-dioxido-3,4-dihydro-2H-benzo[e][1,2,4]thiadiazin-2-yl)-3-(6-fluoro-2,3-dimethylphenyl)butanoic acid

To a stirred solution tert-butyl (2S)-2-((4-chloro-2-methyl-6-(methylamino) phenyl) sulfonamide)-3-(6-fluoro-2,3-dimethylphenyl) butanoate (285 mg, 0.55 mmol, 1 equiv) in dioxane (11 mL) were added TsOH (95 mg, 0.55 mmol, 1 equiv) and 1,3,5-trioxane (496 mg, 5.51 mmol, 10 equiv) at room temperature. The resulting mixture was stirred overnight at 110° C. The resulting mixture was concentrated under vacuum. The residue was purified by reverse flash chromatography with the following conditions: column, C18 silica gel; mobile phase, MeCN in Water (0.1% FA), 10% to 100% gradient in 10 min; detector, UV 254 nm. This resulted in of(2S)-2-(6-chloro-4,8-dimethyl-1,1-dioxido-3,4-dihydro-2H-benzo[e][1,2,4]thiadiazin-2-yl)-3-(6-fluoro-2,3-dimethylphenyl)butanoic acid (47 mg, 18.8%).

### Step 3: Synthesis of 5-((1S,2R)-1-(6-chloro-4,8-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added (2S)-2-(6-chloro-4,8-dimethyl-1,1-dioxido-3,4-dihydro-2H-benzo[e][1,2,4]thiadiazin-2-yl)-3-(6-fluoro-2,3-dimethylphenyl)butanoic acid (60 mg, 0.13 mmol, 1 equiv) in THF (1 mL). To the above mixture was added CDI (32.1 mg, 0.2 mmol, 1.5 equiv) in portions at room temperature. The resulting mixture was stirred for additional 30 min at room temperature. To the above mixture was added N₂H₄·H₂O (19 μL, 0.4 mmol, 3 equiv) dropwise at 0° C. The resulting mixture was stirred 30 min at 0° C. The reaction was quenched with water at 0° C. The resulting mixture was extracted with EtOAc (2×10 mL). The combined organic layers were washed with brine (1×10 mL), dried over anhydrous Na₂SO₄. After filtration, the filtrate was concentrated under reduced pressure to afford crude product SM1. Into an 8 mL vial were added SM1 in dioxane (1 mL). To the above mixture was added CDI (53.5 mg, 0.33 mmol, 2.5 equiv) in portions at room temperature. The resulting mixture was stirred for additional 30 min at room temperature. The crude product was used in the next step directly without further purification.

The crude product (60 mg) was purified by Prep-HPLC with the following conditions (Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 25% B to 55% B in 9 min, 55% B; Wave Length: 220 nm; RT1 (min): 7) to afford 5-((1S,2R)-1-(6-chloro-4,8-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (10.3 mg, 17.13%). LCMS:(ES, m/z): [M−H]⁺=493.05. ¹H NMR (300 MHz, Methanol-d₄) δ 6.99 (s, 1H), 6.75-6.65 (m, 2H), 6.54 (d, J=2.5 Hz, 1H), 5.59-5.55 (m, 1H), 5.27-5.22 (d, J=13.8 Hz, 1H), 4.96 (s, 1H), 3.90 (s, 1H), 2.84 (s, 3H), 2.59 (s, 3H), 2.39 (s, 3H), 2.23 (s, 3H), 1.45-1.43 (m, 3H).

### Example 27: -((1S,2R)-1-(7-chloro-1,1-dioxido-3,4-dihydro-2H-pyrido[4,3-b][1,4,5]oxathiazepin-2-yl)-2-(6-fluoro-2,3-dimethylphenyl)propyl)-1,3,4-oxadiazol-2 (3H)-one

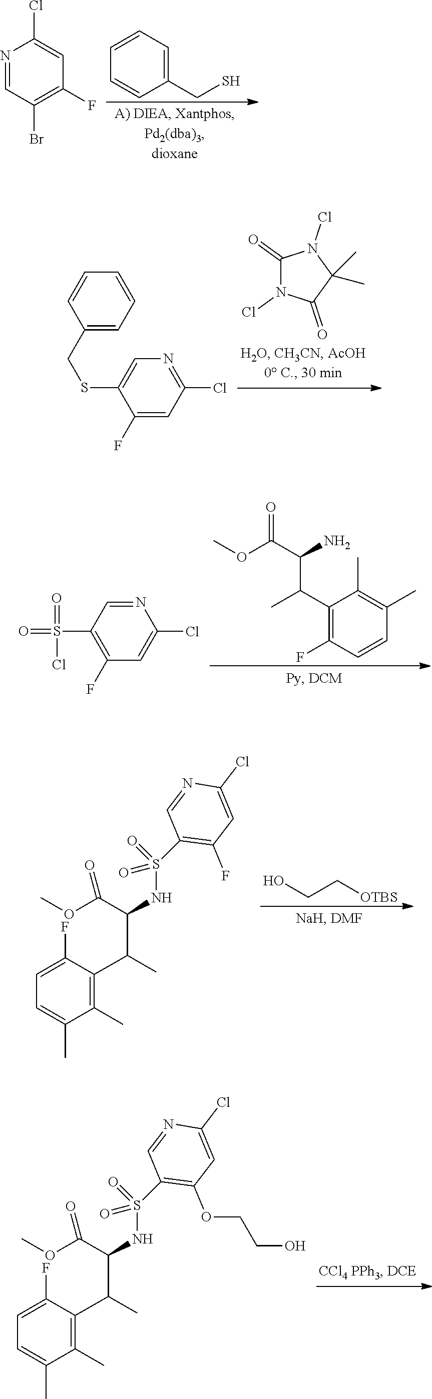

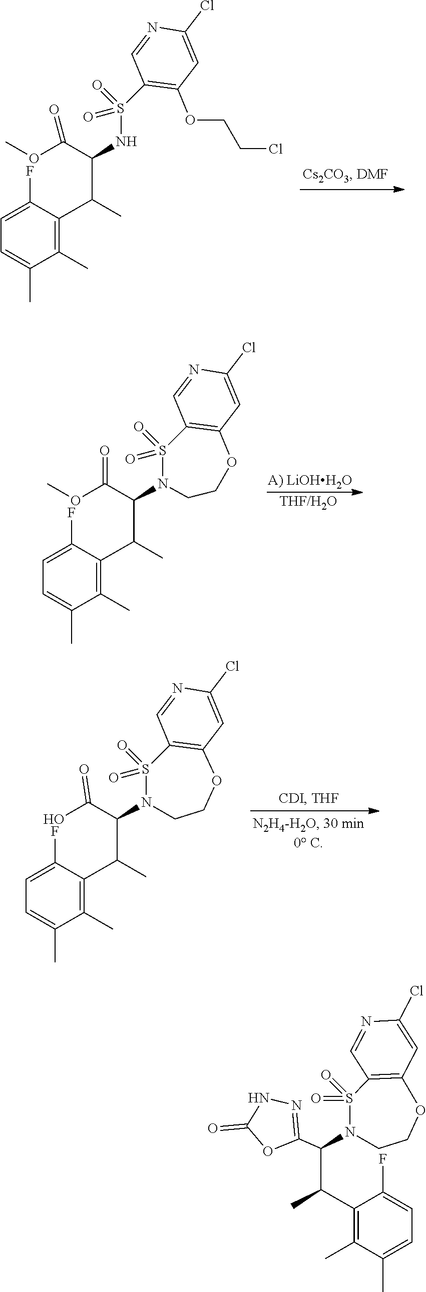

### Step 1: Synthesis of 5-(benzylthio)-2-chloro-4-fluoropyridine

To a stirred solution of 5-bromo-2-chloro-4-fluoropyridine (1 g, 4.75 mmol, 1 equiv) and dioxane (10 mL) was added DIEA (2.48 mL, 14.2 mmol, 3 equiv) Xantphos (550 mg, 0.95 mmol, 0.2 equiv) Pd₂(dba)₃ (435 mg, 0.48 mmol, 0.1 equiv)benzyl mercaptan (669 μL, 5.7 mmol, 1.2 equiv) dropwise at room temperature. The resulting mixture was stirred for 4 h at 100° C. under nitrogen atmosphere. The reaction was quenched with water at room temperature. The resulting mixture was extracted with EtOAc (3×50 mL). The combined organic layers were washed with brine (2×200 mL), dried over anhydrous Na₂SO₄.

After filtration, the filtrate was concentrated under reduced pressure. The residue was purified by reverse flash chromatography with the following conditions: column, C18 silica gel; mobile phase, MeCN in Water (10 mmol/L NH₄HCO₃), 10% to 50% gradient in 20 min; detector, UV 220 nm. This resulted in 5-(benzylsulfanyl)-2-chloro-4-fluoropyridine (550 mg, 45.6%).

### Step 2: Synthesis of 6-chloro-4-fluoropyridine-3-sulfonyl chloride

To a stirred solution of 5-(benzylsulfanyl)-2-chloro-4-fluoropyridine (1.34 g, 5.28 mmol, 1 equiv) and H₂O (900 μL) AcOH (1.51 mL, 26 mmol, 5 equiv) in MeCN was added 1,3-dichloro-5,5-dimethylimidazolidine-2,4-dione (2.08 g, 10.6 mmol, 2 equiv) in portions at 0° C. The resulting mixture was stirred for 30 min at 0° C. The reaction was quenched with Water/Ice at room temperature. The resulting mixture was extracted with EtOAc (2×10 mL). The combined organic layers were washed with brine (2×20 mL), dried over anhydrous Na₂SO₄. After filtration, the filtrate was concentrated under reduced pressure. The crude product was used in the next step directly without further purification.

### Step 3: Synthesis of methyl (2S)-2-((6-chloro-4-fluoropyridine)-3-sulfonamido)-3-(6-fluoro-2,3-dimethylphenyl) butanoate

To a stirred solution of methyl (2S)-2-amino-3-(6-fluoro-2,3-dimethylphenyl) butanoate (1.66 g, 6.94 mmol, 1.00 equiv) and Pyridine (2.81 mL, 34.8 mmol, 5 equiv) was added 6-chloro-4-fluoropyridine-3-sulfonyl chloride (1.6 g, 7 mmol, 1.00 equiv) in DCM (20 mL) dropwise at 0° C. The resulting mixture was stirred overnight at room temperature. The resulting mixture was extracted with DCM (3×20 mL). The combined organic layers were washed with brine (3×50 mL), dried over anhydrous MgSO₄. After filtration, the filtrate was concentrated under reduced pressure. The residue was purified by silica gel column chromatography, eluted with PE/EtOAc (9:1) to afford of methyl (2S)-2-((6-chloro-4-fluoropyridine)-3-sulfonamido)-3-(6-fluoro-2,3-dimethylphenyl) butanoate (1.56 g, 51.8%).

### Step 4: Synthesis of methyl (2S)-2-((6-chloro-4-(2-hydroxyethoxy) pyridine)-3-sulfonamido)-3-(6-fluoro-2,3-dimethylphenyl) butanoate

To a stirred solution of methyl (2S)-2-((6-chloro-4-fluoropyridine)-3-sulfonamido)-3-(6-fluoro-2,3-dimethylphenyl) butanoate (710 mg, 1.64 mmol, 1 equiv) and 2-[(tert-butyldimethylsilyl) oxy]ethanol (650 μL, 3.28 mmol, 2.0 equiv) in DMF was added NaH (200 mg, 4.92 mmol, 3 equiv, 60%) in portions at 0° C. The resulting mixture was stirred for 3 h at room temperature. The reaction was quenched with water at room temperature. The residue was purified by reverse flash chromatography with the following conditions: column, C18 silica gel; mobile phase, MeCN in Water (0.1% FA), 0% to 100% gradient in 30 min; detector, UV 220 nm. This resulted in of methyl (2S)-2-((6-chloro-4-(2-hydroxyethoxy) pyridine)-3-sulfonamido)-3-(6-fluoro-2,3-dimethylphenyl) butanoate (190 mg, 24.4%).

### Step 5: Synthesis of methyl (2S)-2-((6-chloro-4-(2-chloroethoxy) pyridine)-3-sulfonamido)-3-(6-fluoro-2,3-dimethylphenyl) butanoate

To a stirred solution of methyl (2S)-2-((6-chloro-4-(2-hydroxyethoxy) pyridine)-3-sulfonamido)-3-(6-fluoro-2,3-dimethylphenyl) butanoate (180 mg, 0.38 mmol, 1 equiv) and PPh₃ (497 mg, 1.9 mmol, 2 equiv) in DCE (6 mL) was added CCl₄ (137 μL, 1.42 mmol, 1.5 equiv) dropwise at room temperature. The resulting mixture was stirred 1 h at 80° C. The resulting mixture was concentrated under reduced pressure.

The residue was purified by reverse flash chromatography with the following conditions: column, C18 silica gel; mobile phase, MeCN in Water (10 mmol/L NH₄HCO₃), 0% to 10 0% gradient in 30 min; detector, UV 220 nm. This resulted in methyl (2S)-2-((6-chloro-4-(2-chloroethoxy) pyridine)-3-sulfonamido)-3-(6-fluoro-2,3-dimethylphenyl) butanoate (160 mg, 85.6%).

### Step 6: Synthesis of methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoate

To a stirred solution of methyl (2S)-2-((6-chloro-4-(2-chloroethoxy) pyridine)-3-sulfonamido)-3-(6-fluoro-2,3-dimethylphenyl) butanoate (260 mg, 0.53 mmol, 1 equiv) in DMF is added Cs₂CO₃ (343 mg, 1.05 mmol, 2 equiv) at room temperature. The resulting mixture was stirred 1 h at 60° C. The resulting mixture was extracted with EtOAc (2×50 mL). The combined organic layers were washed with brine (2×50 mL), dried over anhydrous Na₂SO₄. After filtration, the filtrate was concentrated under reduced pressure. The crude product was purified by Prep-HPLC with the following conditions (PE:EtOAc 1:1) to afford methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoate (190 mg, 78.91%).

### Step 7: Synthesis of (2S)-2-(7-chloro-1,1-dioxido-3,4-dihydro-2H-pyrido[4,3-b][1,4,5]oxathiazepin-2-yl)-3-(6-fluoro-2,3-dimethylphenyl) butanoic acid

To a stirred solution of methyl (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoate (5 mg, 0.011 mmol, 1 equiv) and H₂O (1 mL) in THF was added LiOH·H₂O (87 mg, 2.08 mmol, 5 equiv) at room temperature. The resulting mixture was stirred for 30 min at room temperature. The mixture/residue was acidified to pH 6 with conc. HCl. The resulting mixture was extracted with EtOAc (3×20 mL). The combined organic layers were washed with brine (2×50 mL), dried over anhydrous Na₂SO₄. After filtration, the filtrate was concentrated under reduced pressure. The crude product/resulting mixture was used in the next step directly without further purification.

### Step 8: Synthesis of 5-((1S,2R)-1-(7-chloro-1,1-dioxido-3,4-dihydro-2H-pyrido[4,3-b][1,4,5]oxathiazepin-2-yl)-2-(6-fluoro-2,3-dimethylphenyl)propyl)-1,3,4-oxadiazol-2 (3H)-one

To a stirred solution of (2S)-2-(7-chloro-1,1-dioxido-3,4-dihydro-2H-pyrido[4,3-b][1,4,5]oxathiazepin-2-yl)-3-(6-fluoro-2,3-dimethylphenyl)butanoic acid (160 mg, 0.361 mmol, 1 equiv) in THF was added CDI (234 mg, 1.44 mmol, 4 equiv) at room temperature. The resulting mixture was stirred 30 min at room temperature. To the above mixture was added NH₂NH₂·H₂O (54.3 mg, 1.08 mmol, 3 equiv) at 0° C. The resulting mixture was stirred for additional 30 min at 0° C. The reaction was quenched with Water/Ice at room temperature. The resulting mixture was extracted with EtOAc (2×50 mL). The combined organic layers were washed with brine (2×50 mL), dried over anhydrous Na₂SO₄. After filtration, the filtrate was concentrated under reduced pressure. To the above mixture was added CDI (234 mg, 1.44 mmol, 4 equiv) at room temperature. The resulting mixture was stirred for additional 30 min at room temperature. The reaction was quenched with water at room temperature. The residue was purified by reverse flash chromatography with the following conditions: Column: Xselect CSH C18 OBD Column 30*150 mm 5 μm, n; Mobile Phase A: Water (0.1% FA), Mobile Phase B: ACN; Flow rate: 60 mL/min; Gradient: 15% B to 45% B in 8 min, 45% B; Wave Length: 254 nm; RT1 (min): 7. This resulted in 5-((1S,2R)-1-(7-chloro-1,1-dioxido-3,4-dihydro-2H-pyrido[4,3-b][1,4,5]oxathiazepin-2-yl)-2-(6-fluoro-2,3-dimethylphenyl)propyl)-1,3,4-oxadiazol-2 (3H)-one (30.9 mg, 17.6%). LCMS:(ES, m/z): 482.95. ¹H NMR (300 MHz, Methanol-d₄) δ 6.99 (s, 1H), 6.75-6.65 (m, 2H), 6.54 (d, J=2.5 Hz, 1H), 5.59-5.55 (m, 1H), 5.27-5.22 (d, J=13.8 Hz, 1H), 4.96 (s, 1H), 3.90 (s, 1H), 2.84 (s, 3H), 2.59 (s, 3H), 2.39 (s, 3H), 2.23 (s, 3H), 1.45-1.43 (m, 3H). ¹H NMR (300 MHz, Methanol-d₄) δ 8.67 (d, J=2.4 Hz, 1H), 7.08-7.02 (d, J=2.5 Hz, 1H), 7.00-6.98 (t, J=7.3 Hz, 1H), 6.76-6.69 (t, J=10.5 Hz, 1H), 5.55-5.51 (d, J=11.7 Hz, 1H), 4.97-4.92 (m, 1H), 4.48-4.44 (d, J=13.5 Hz, 1H), 3.99-3.96 (d, J=12.0 Hz, 1H), 3.84-3.79 (m, 2H), 2.35-2.31 (s, 3H), 2.21-2.16 (s, 3H), 1.45-1.43 (d, J=6.9 Hz, 3H).

### Example 28 and 29: 5-((1S,2R)-1-(7-chloro-6-((S)-1-hydroxyethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

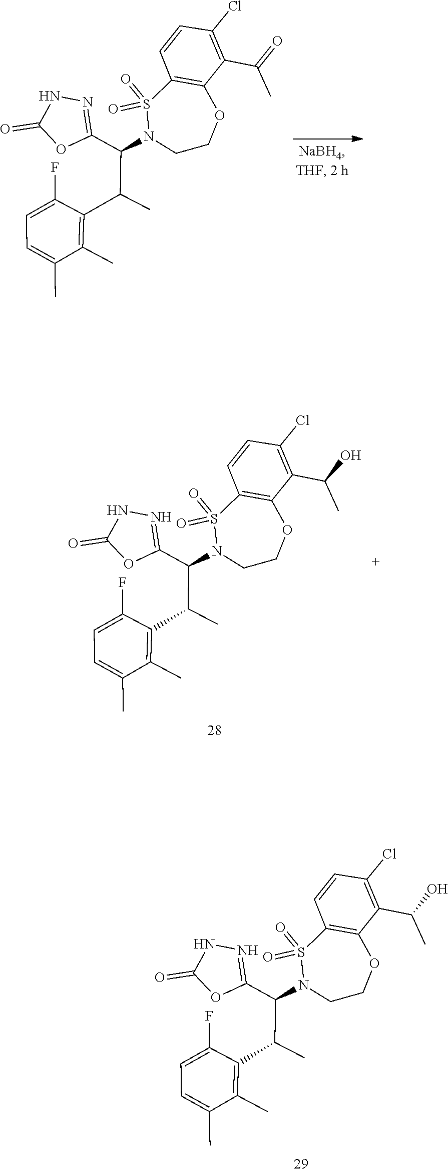 [SMILES: CC(=O)c1c(Cl)ccc2c1OCCN([C@H](c1n[nH]c(=O)o1)C(C)c1c(F)ccc(C)c1C)S2(=O)=O ; Cc1ccc(F)c([C@@H](C)[C@@H](c2n[nH]c(=O)o2)N2CCOc3c(ccc(Cl)c3[C@H](C)O)S2(=O)=O)c1C ; Cc1ccc(F)c([C@@H](C)[C@@H](c2n[nH]c(=O)o2)N2CCOc3c(ccc(Cl)c3[C@@H](C)O)S2(=O)=O)c1C]

Into a 8 mL sealed tube were added 5-((1S)-1-(6-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (from example 25) (100 mg, 0.19 mmol, 1 equiv), THF (1 mL) and sodium borohydride (14.1 mg, 0.38 mmol, 2 equiv) at room temperature. The resulting mixture was stirred for 10 min at room temperature. The crude product was purified by Chiral-Prep-HPLC with the following conditions: Column: XselectCSH C18 OBDColumn 30*150 mm 5 μm, n; Mobile Phase A: Water (0.1% FA), Mobile Phase B: ACN; Flow rate: 60 mL/min; Gradient: 15% B to 45% B in 8 min, 45% B; Wave Length: 254 nm; RT1 (min): 7. This resulted in 2 diastereoisomers:

Example 28-((1S,2R)-1-(7-chloro-6-((S)-1-hydroxyethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one(5.4 mg, 5.2%). LCMS: (ES, m/z): (M−H)=526.10. ¹H NMR (300 MHz, Methanol-d₄) δ7.72-7.65 (m, 2H), 7.30-7.20 (dd, J=23.1, 8.5 Hz, 1H), 6.99-6.95 (ddd, J=14.5, 8.4, 5.7 Hz, 1H), 6.78-6.71 (dd, J=11.7, 8.4 Hz, 1H), 5.52-5.41 (dq, J=10.1, 6.7 Hz, 1H), 5.09-5.00 (dd, J=16.9, 11.1 Hz, 1H), 4.57-4.38 (m, 1H), 4.19-4.15 (m, 1H), 4.06-3.91 (m, 2H), 3.76-3.70 (m, 1), 2.27 (d, J=2.4 Hz, 3H), 2.20-2.19 (d, J=6.3 Hz, 3H), 1.58-1.56 (dd, J=6.7, 2.4 Hz, 3), 1.40-1.36 (ddd, J=7.0, 4.9, 1.2 Hz, 3H).

Example 29((1S,2R)-1-(7-chloro-6-((R)-1-hydroxyethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one(6.9 mg, 6.56%). LCMS: (ES, m/z): (M−H)=526.10. ¹H NMR (300 MHz, Methanol-d₄) δ 7.73-7.65 (m, 2H), 7.32-7.20 (d, J=8.5 Hz, 1H), 7.03-6.95 (m, 1H), 6.78-6.72 (dd, J=11.9, 8.3 Hz, 1H), 5.48-5.41 (q, J=6.8 Hz, 1H), 5.10-5.03 (d, J=11.3 Hz, 1H), 4.46-4.41 (d, J=10.8 Hz, 1H), 4.22-4.13 (q, J=12.6, 11.0 Hz, 2H), 3.99-3.94 (m, 1H), 3.76-3.70 (dd, J=11.9, 6.6 Hz, 1H), 2.28 (d, J=4.8 Hz, 3H), 2.19 (d, J=6.4 Hz, 3H), 1.59-1.56 (dd, J=8.3, 6.7 Hz, 3H), 1.37-1.35 (m, 3H).
Note: the stereochemistry of the chiral center bearing the hydroxy has been arbitrary assigned.

### Example 30: 5-((1S,2R)-1-(6-chloro-4-(3-methoxypropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

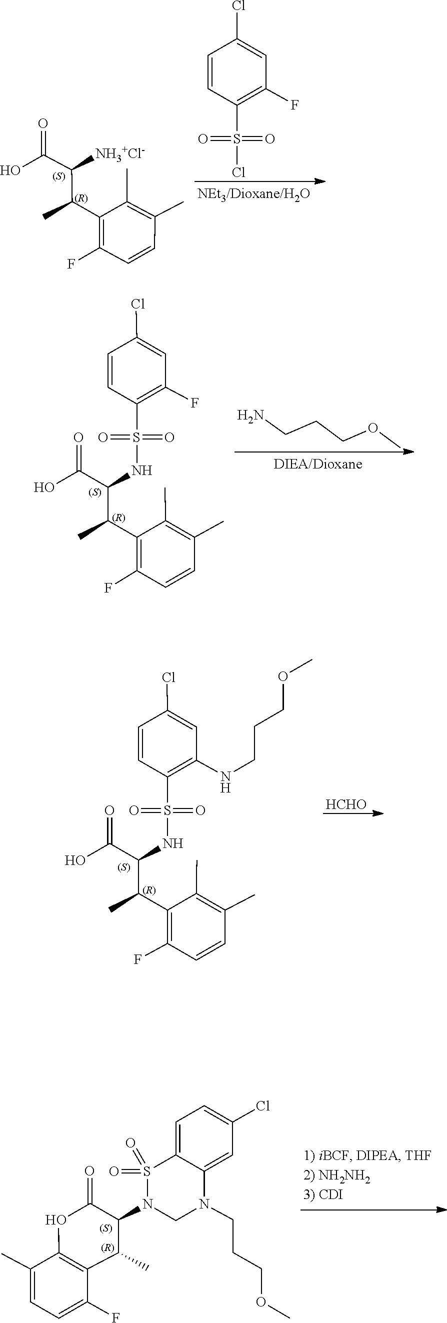

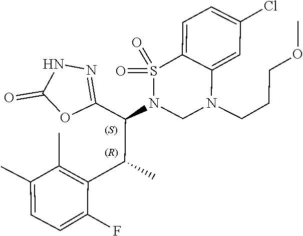

### Step 1: Synthesis of (2S,3R)-2-(4-chloro-2-fluorobenzenesulfonamido)-3-(6-fluoro-2,3-dimethylphenyl) butanoic acid

(2S,3R)-2-amino-3-(6-fluoro-2,3-dimethylphenyl) butanoic acid hydrochloride (2.0 g, 7.64 mmol, 1.0 eq) was suspended in a mixture of water and dioxane (v/v=1/1, 76 mL). To this, triethylamine (3.2 mL, 22.9 mmol, 3.0 eq) was added and the solution was cooled to 0° C., after which 4-chloro-2-fluorobenzenesulfonyl chloride (1.93 g, 8.4 mmol, 1.1 eq) was added portion wise. The reaction was continued at room temperature overnight. When the reaction was completed, it was cooled down and slowly acidified with 1M HCl. The mixture was extracted with AcOEt. Combined organic layers were washed with brine, dried over Na₂SO₄, filtered, and concentrated under reduced pressure. Obtained oily residue was purified by FCC (0-4% MeOH in DCM) to afford (2S,3R)-2-(4-chloro-2-fluorobenzenesulfonamido)-3-(6-fluoro-2,3-dimethylphenyl) butanoic acid (3.2 g, 7.6 mmol, yield 100%).

### Step 2: Synthesis of (2S,3R)-2-{4-chloro-2-[(3-methoxypropyl) amino]benzenesulfonamido}-3-(6-fluoro-2,3-dimethylphenyl)butanoic acid

(2S,3R)-2-(4-chloro-2-fluorobenzenesulfonamido)-3-(6-fluoro-2,3-dimethylphenyl) butanoic acid (112 mg, 0.27 mmol, 1.0 eq) was dissolved in anhydrous dioxane (2.7 mL). N, N-Diisopropylethylamine (DIPEA) (103 μL, 0.59 mmol, 2.2 eq) and 3-Methoxypropylamine (359 mg, 4 mmol, 15.0 eq) were added to the solution. The reaction mixture was stirred at 80° C. overnight. Upon completion the mixture was poured into 10% aq NaH₂PO₄ solution and extracted with EtOAc. The combined organic layers were washed with brine and dried over sodium sulfate. The solvent was removed under reduced pressure to give (2S,3R)-2-{4-chloro-2-[(3-methoxypropyl) amino]benzenesulfonamido}-3-(6-fluoro-2,3-dimethylphenyl) butanoic acid (146 mg, 0.27 mmol, yield 100%), which was used in the next step without further purification.

### Step 3: Synthesis of (2S,3R)-2-(6-chloro-4-(3-methoxypropyl)-1,1-dioxido-3,4-dihydro-2H-benzo[e][1,2,4]thiadiazin-2-yl)-3-(6-fluoro-2,3-dimethylphenyl) butanoic acid

(2S,3R)-2-{4-chloro-2-[(3-methoxypropyl)amino]benzenesulfonamido}-3-(6-fluoro-2,3-dimethylphenyl)butanoic acid (116 mg, 0.21 mmol, 1.0 eq) was dissolved in anhydrous dioxane (5.8 mL), followed by the addition of paraformaldehyde (32 mg, 1.07 mmol, 5.0 eq) and 4M HCl in dioxane (214 μL, 0.43 mmol, 2.0 eq) and the resulting mixture was allowed to stir at 60° C. overnight. After the reaction was complete, the mixture was cooled down, diluted with water, and extracted using EtOAc. The organics were combined, washed with brine, and dried over MgSO₄. Solvents were removed in vacuo. The residue was purified by FCC (0-6% MeOH in DCM) to give 2S,3R)-2-(6-chloro-4-(3-methoxypropyl)-1,1-dioxido-3,4-dihydro-2H-benzo[e][1,2,4]thiadiazin-2-yl)-3-(6-fluoro-2,3-dimethylphenyl) butanoic acid (119 mg, 0.21 mmol, yield 99%).

### Step 4: Synthesis of 5-((1S,2R)-1-(6-chloro-4-(3-methoxypropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

(2S,3R)-2-(6-chloro-4-(3-methoxypropyl)-1,1-dioxido-3,4-dihydro-2H-benzo[e][1,2,4]thiadiazin-2-yl)-3-(6-fluoro-2,3-dimethylphenyl) butanoic (69 mg, 0.12 mmol, 1.0 eq) was dissolved in anhydrous THF (2 mL) and cooled to −10° C. DIEA (24 μL, 0.135 mmol, 1.1 eq) was added, followed by dropwise addition of isobutyl-chloroformate (20 μL, 0.147 mmol, 1.2 eq). The reaction was continued at −10° C. for 30 min. Upon complete consumption of substrate, hydrazine monohydrate (31 μL, 0.614 mmol, 5.0 eq) was added dropwise and stirring was continued for 1 h. After formation of hydrazide was complete, CDI (159 mg, 0.982 mmol, 8.0 eq) was added portion wise and the reaction was continued at ambient temperature for 1 h. The mixture was poured into water and extracted with EtOAc. Organic layers were combined, washed with brine, dried over sodium sulfate and the solvent was removed under reduced pressure. The residue was purified by pTLC (2% MeOH in DCM) to afford the title compound (24 mg, 0.044 mmol, yield 36%). ¹H NMR (400 MHz, Methanol-d₄) δ 7.56 (d, J=8.4 Hz, 1H), 6.96 (dd, J=8.4, 5.7 Hz, 1H), 6.78 (d, J=1.8 Hz, 1H), 6.75 (dd, J=8.4, 1.8 Hz, 1H), 6.69 (dd, J=12.1, 8.4 Hz, 1H), 5.43 (d, J=11.9 Hz, 1H), 5.37 (d, J=14.5 Hz, 1H), 5.05 (d, J=14.5 Hz, 1H), 3.88 (dq, J=13.3, 6.9 Hz, 1H), 3.53 (dt, J=15.4, 6.7 Hz, 1H), 3.40 (t, J=5.7 Hz, 2H), 3.34 (s, 3H), 3.29-3.21 (m, 1H), 2.38 (s, 3H), 2.21 (s, 3H), 1.83 (p, J=6.5 Hz, 2H), 1.45 (dd, J=6.9, 1.1 Hz, 3H). LC-MS: m/z=536.93 [M−H]−.

### Example 31: 5-((1S,2R)-1-(6-chloro-5-methyl-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

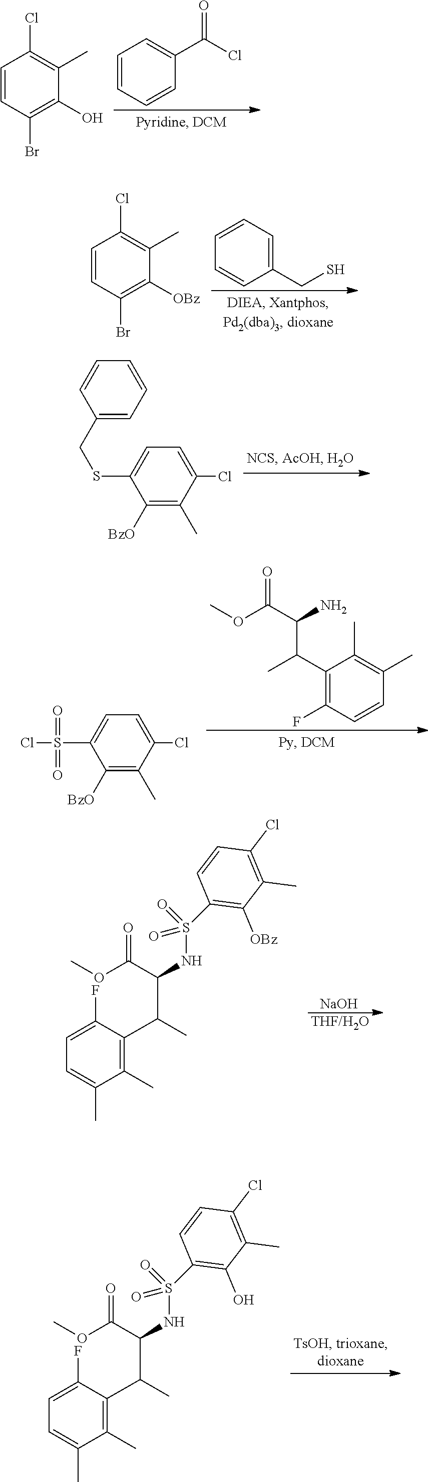

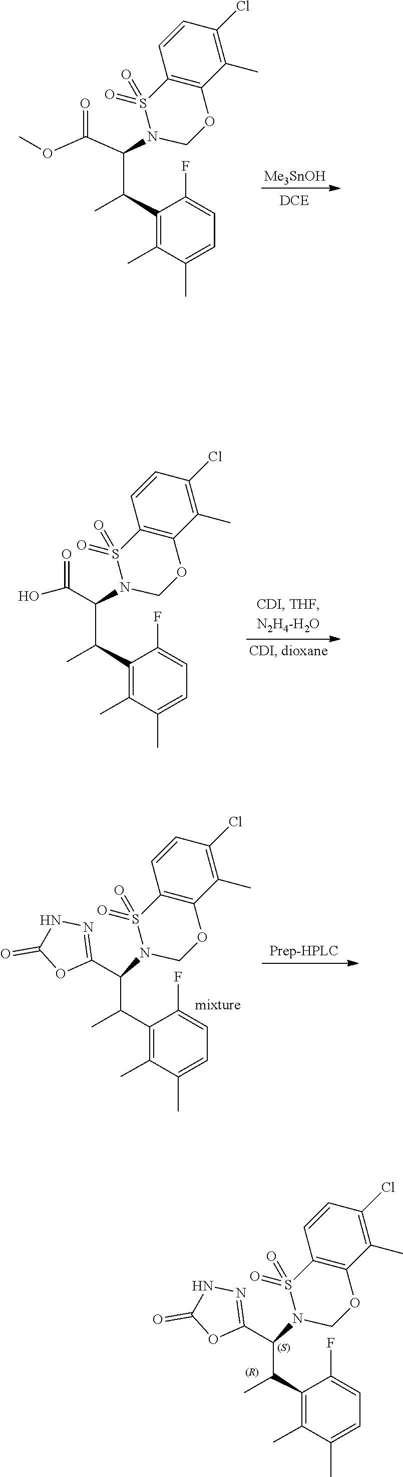

### Step 1: Synthesis of 6-bromo-3-chloro-2-methylphenyl benzoate

Into a 100 mL round-bottom flask were added 6-bromo-3-chloro-2-methylphenol (2.2 g, 10 mmol, 1 equiv), DCM, pyridine (1.6 mL, 20 mmol, 2 equiv) and benzoyl chloride (6.9 g, 50 mmol, 5 equiv) at room temperature. The resulting mixture was stirred for 1 h at room temperature under air atmosphere. The resulting mixture was concentrated under reduced pressure. The residue was purified by reverse flash chromatography with the following conditions: column, C18 silica gel; mobile phase, MeCN in Water (0.1% FA), 60% to 70% gradient in 10 min; detector, UV 254 nm. This resulted in 6-bromo-3-chloro-2-methylphenyl benzoate (3 g, 94.0%).

### Step 2: Synthesis of 6-(benzylsulfanyl)-3-chloro-2-methylphenyl benzoate

Into a 40 mL vial were added 6-bromo-3-chloro-2-methylphenyl benzoate (2.5 g, 7.7 mmol, 1 equiv), dioxane (25 mL), DIEA (2.7 mL, 15.4 mmol, 2 equiv), Xantphos (444 mg, 0.77 mmol, 0.1 equiv), Pd₂(dba)₃ (492 mg, 0.54 mmol, 0.07 equiv) and benzyl mercaptan (1.1 mL, 9.2 mmol, 1.2 equiv) at room temperature. The resulting mixture was stirred for 2 h at 100° C. under nitrogen atmosphere. The resulting mixture was quenched with water and extracted with EtOAc (2×100 mL). The combined organic layers were washed with brine (1×100 mL), dried over anhydrous Na₂SO₄. After filtration, the filtrate was concentrated under reduced pressure. The residue was purified by Prep-TLC (PE/EtOAc 10:1) to afford 6-(benzylsulfanyl)-3-chloro-2-methylphenyl benzoate (2.8 g, 99/a).

### Step 3: Synthesis of 3-chloro-6-(chlorosulfonyl)-2-methylphenyl benzoate

Into a 100 mL round-bottom flask were added 6-(benzylsulfanyl)-3-chloro-2-methylphenyl benzoate (2.8 g, 7.6 mmol, 1 equiv), AcOH (30 mL) and H₂O (10 mL) at room temperature. To the above mixture was added NCS (2.0 g, 15.2 mmol, 2 equiv) in portions at 0° C. The resulting mixture was stirred for additional 2 h at room temperature. The resulting mixture was extracted with EtOAc (2×100 mL). The combined organic layers were washed with brine (1×100 mL), dried over anhydrous Na₂SO₄. After filtration, the filtrate was concentrated under reduced pressure. This resulted in 3-chloro-6-(chlorosulfonyl)-2-methylphenyl benzoate (3.3 g, 126%) as a colorless crude oil used as is for the next step.

### Step 4: Synthesis of 3-chloro-6-(N-((2S)-3-(6-fluoro-2,3-dimethylphenyl)-1-methoxy-1-oxobutan-2-yl) sulfamoyl)-2-methylphenyl benzoate

Into a 100 mL round-bottom flask were added methyl (2S)-2-amino-3-(6-fluoro-2,3-dimethylphenyl) butanoate (1.2 g, 5 mmol, 1 equiv), DCM (15 mL) and pyridine (2.5 mL, 31.3 mmol, 5 equiv) at room temperature. To the above mixture was added 3-chloro-6-(chlorosulfonyl)-2-methylphenyl benzoate (3.6 g, 6.3 mmol, 1 equiv, 60/6) in portions at 0° C. The resulting mixture was stirred for additional 2 h at room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 3-chloro-6-(N-((2S)-3-(6-fluoro-2,3-dimethylphenyl)-1-methoxy-1-oxobutan-2-yl) sulfamoyl)-2-methylphenyl benzoate (1 g, 29.1%).

### Step 5: Synthesis of (2S)-2-(4-chloro-2-hydroxy-3-methylbenzenesulfonamido)-3-(6-fluoro-2,3-dimethylphenyl) butanoate

Into a 100 mL round-bottom flask were added 3-chloro-6-(N-((2S)-3-(6-fluoro-2,3-dimethylphenyl)-1-methoxy-1-oxobutan-2-yl) sulfamoyl)-2-methylphenyl benzoate (1 g, 1.8 mmol, 1 equiv), THF (10 mL), H₂O (2 mL) and NaOH (210 mg, 5.3 mmol, 2.9 equiv) at room temperature. The resulting mixture was stirred for 1 h at room temperature under air atmosphere. The resulting mixture was extracted with EtOAc (2×50 mL). The combined organic layers were washed with brine (1×100 mL), dried over anhydrous Na₂SO₄. After filtration, the filtrate was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in methyl (2S)-2-(4-chloro-2-hydroxy-3-methylbenzenesulfonamido)-3-(6-fluoro-2,3-dimethylphenyl) butanoate (249 mg, 30.7%).

### Step 6: Synthesis of methyl (2S)-2-(6-chloro-5-methyl-1,1-dioxidobenzo[e][1,4,3]oxathiazin-2 (3H)-yl)-3-(6-fluoro-2,3-dimethylphenyl)butanoate

Into a 20 mL sealed tube were added methyl (2S)-2-(4-chloro-2-hydroxy-3-methylbenzenesulfonamido)-3-(6-fluoro-2,3-dimethylphenyl) butanoate (230 mg, 0.52 mmol, 1 equiv), Dioxane (5 mL), TsOH (93.1 mg, 0.54 mmol, 1 equiv) and trioxane (730 mg, 8.1 mmol, 15 equiv) at room temperature. The resulting mixture was stirred for 3 days at 100° C. under air atmosphere. The resulting mixture was filtered, the filter cake was washed with EtOAc (2×10 mL). The filtrate was concentrated under reduced pressure. The residue was purified by reverse flash chromatography with the following conditions: column, C18 silica gel; mobile phase, MeCN in Water (0.1% FA), 60% to 70% gradient in 10 min; detector, UV 254 nm. This resulted in methyl (2S)-2-(6-chloro-5-methyl-1,1-dioxidobenzo[e][1,4,3]oxathiazin-2 (3H)-yl)-3-(6-fluoro-2,3-dimethylphenyl)butanoate (160 mg, 64.9%).

### Step 7: Synthesis of (2S)-2-(6-chloro-5-methyl-1,1-dioxidobenzo[e][1,4,3]oxathiazin-2 (3H)-yl)-3-(6-fluoro-2,3-dimethylphenyl)butanoic acid

Into a 40 mL vial were added methyl (2S)-2-(6-chloro-5-methyl-1,1-dioxidobenzo[e][1,4,3]oxathiazin-2 (3H)-yl)-3-(6-fluoro-2,3-dimethylphenyl)butanoate (123 mg, 0.27 mmol, 1 equiv), DCE (4.92 mL) and trimethyltin hydroxide (488 mg, 2.7 mmol, 10 equiv) at room temperature. The resulting mixture was stirred overnight at 60° C. under air atmosphere. The resulting mixture was concentrated under reduced pressure. The residue was purified by reverse flash chromatography with the following conditions: column, C18 silica gel; mobile phase, MeCN in Water (0.1% FA), 80% to 90% gradient in 10 min; detector, UV 254 nm. This resulted in (2S)-2-(6-chloro-5-methyl-1,1-dioxidobenzo[e][1,4,3]oxathiazin-2 (3H)-yl)-3-(6-fluoro-2,3-dimethylphenyl)butanoic acid (100 mg, 83.9%).

### Step 8: Synthesis of 5-((1S,2R)-1-(6-chloro-5-methyl-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

Into a 25 mL round-bottom flask were added (2S)-2-(6-chloro-5-methyl-1,1-dioxidobenzo[e][1,4,3]oxathiazin-2 (3H)-yl)-3-(6-fluoro-2,3-dimethylphenyl)butanoic acid (100 mg, 0.23 mmol, 1 equiv), THF (2 mL) and CDI (110 mg, 0.68 mmol, 3 equiv) at room temperature. The resulting mixture was stirred for 0.5 h at room temperature under air atmosphere. To the above mixture was added NH₂NH₂·H₂O (33 μL, 0.68 mmol, 3 equiv) dropwise at 0° C. The resulting mixture was stirred for additional 0.5 h at 0° C. The resulting mixture was extracted with EtOAc (2×30 mL). The combined organic layers were washed with brine (1×30 mL), dried over anhydrous Na₂SO₄. After filtration, the filtrate was concentrated under reduced pressure. Into a 25 mL round-bottom flask were added the above crude product, dioxane (2 mL) and CDI (110 mg, 0.68 mmol, 3 equiv) at room temperature. The resulting mixture was stirred for 0.5 h at room temperature under air atmosphere. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 5-((1S)-1-(6-chloro-5-methyl-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (30 mg, 27.5%).

The solid was further purified by reversed-phase flash chromatography with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 50% B in 10 min, 50% B; Wave Length: 254 nm; RT1 (min): 9.1, 9.2 (min). This resulted in 5-((1S,2R)-1-(6-chloro-5-methyl-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (2 mg, 4.5%). LCMS:(ES, m/z): [M−H]⁺=480.05. ¹H NMR (300 MHz, Methanol-d₄) δ 7.47 (d, J=8.6 Hz, 1H), 7.09 (d, J=8.6 Hz, 1H), 6.91 (dd, J=8.4, 5.8 Hz, 1H), 6.64 (dd, J=12.1, 8.4 Hz, 1H), 5.87 (d, J=1.6 Hz, 2H), 5.48 (dd, J=11.9, 1.6 Hz, 1H), 3.93-3.76 (m, 1H), 2.30 (s, 3H), 2.14 (s, 3H), 2.04 (s, 3H), 1.37 (dd, J=6.9, 1.1 Hz, 3H), 1.29-1.16 (m, 1H).

### Example 32: 5-((1S,2R)-1-(6-chloro-4,5-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

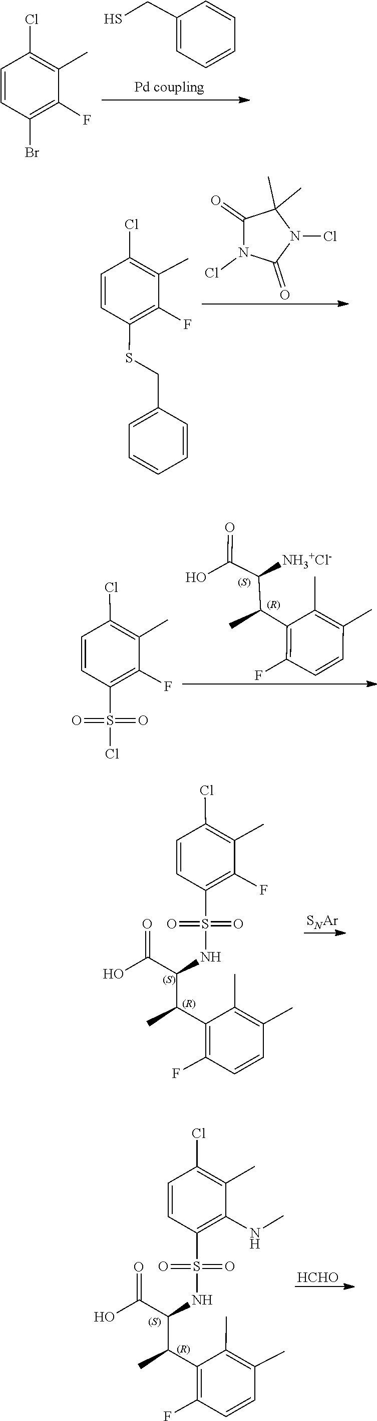

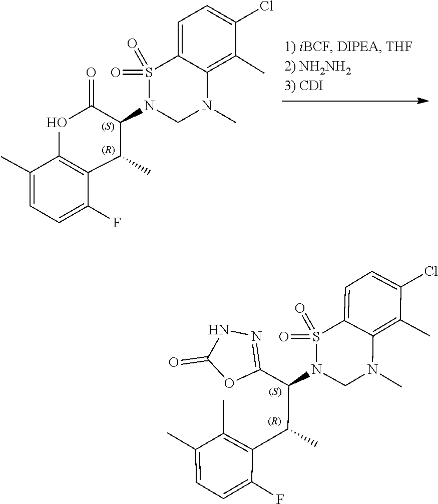

### Step 1: Synthesis of 1-(benzylsulfanyl)-4-chloro-fluoro-3-methylbenzene

To a degassed solution of 1-Bromo-4-chloro-2-fluoro-3-methylbenzene (500 mg, 2.24 mmol, 1.0 eq) in anhydrous dioxane (5.0 mL) were added N,N-diisopropylethylamine (DIPEA) (0.78 mL, 4.8 mmol, 2.0 eq), tris(dibenzylideneacetone)dipalladium(0) [c](5.4 mg, 5 mol %) and 4,5-Bis(diphenylphosphino)-9,9-dimethylxanthene [Xantphos](0.129 g, 0.22 mmol, 10 mol %). The mixture was degassed again and purged with argon, following which the reaction vessel was immersed in an oil bath preheated to 100° C. After 10 mins, benzyl mercaptan (0.27 g, 2.15 mmol, 1 eq) was added dropwise. The reaction was continued at 100° C. overnight. When the reaction was completed the volatiles were removed under reduced pressure. The residue was redissolved in EtOAc and filtered through a pad of silica gel. The filtrate was concentrated and purified by FCC (0-10% of EtOH in hexanes) to afford 1-(benzylsulfanyl)-4-chloro-2-fluoro-3-methylbenzene (0.540 g, 1.92 mmol, 86%).

### Step 2: Synthesis of 4-chloro-2-fluoro-3-methylbenzene-1-sulfonyl chloride

To a solution of 1-(benzylsulfanyl)-4-chloro-2-fluoro-3-methylbenzene (200 mg, 0.71 mmol, 1.0 eq) in MeCN (8.0 mL) and water (0.2 mL) was added glacial acetic acid (51 μL, 0.89 mmol, 1.25 eq), followed by portion wise addition of 1,3-dichloro-5,5-dimethylhydantoin (281 mg, 1.43 mmol, 2.0 eq). The reaction was continued at room temperature and monitored by TLC and LC-MS for the next 1.5 h until starting material was completely consumed. When the reaction was completed, the mixture was poured into water and extracted with DCM. Combined organic layers were dried over Na₂SO₄, filtered, and concentrated in vacuo. Crude material was used in the next step without further purification (170 mg, 0.66 mmol, yield: 93%).

### Step 3: Synthesis of (2S,3R)-2-(4-chloro-2-fluoro-3-methylbenzensulfonamido)-3-(6-fluoro-2,3-dimethylphenyl)butanoic acid

The title compound was synthesized following the procedure described for Ex-30.

### Step 4: Synthesis of (2S,3R)-2-((4-chloro-3-methyl-2-(methylamino) phenyl) sulfonamido)-3-(6-fluoro-2,3-dimethylphenyl)butanoic acid

The title compound was synthesized following the procedure described for Ex-30 using methylamine instead of 3-methoxypropylamine.

### Step 5: Synthesis of (2S,3R)-2-(6-chloro-4,5-dimethyl-1,1-dioxido-3,4-dihydro-2H-benzo[e][1,2,4]thiadiazin-2-yl)-3-(6-fluoro-2,3-dimethylphenyl)butanoic acid

The title compound was synthesized following the procedure described for Ex-30 Step 6: Synthesis of 5-((1S,2R)-1-(6-chloro-4,5-dimethyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-30

¹H NMR (300 MHz, DMSO-d₆) δ 12.19 (s, 1H), 7.60 (d, J=8.5 Hz, 1H), 7.28 (d, J=8.6 Hz, 1H), 7.05 (dd, J=8.4, 5.9 Hz, 1H), 6.85 (dd, J=12.2, 8.4 Hz, 1H), 5.28 (d, J=11.6 Hz, 1H), 4.99 (d, J=14.1 Hz, 1H), 4.89 (d, J=14.1 Hz, 1H), 3.94-3.78 (m, 1H), 2.82 (s, 3H), 2.30 (s, 3H), 2.26 (s, 3H), 2.18 (s, 3H), 1.38 (d, J=6.7 Hz, 3H). LC-MS: m/z=493.13 [M−H]−.

### Example 33:5-((1S,2R)-1-(6-chloro-1,1-dioxido-4-(tetrahydro-2H-pyran-4-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

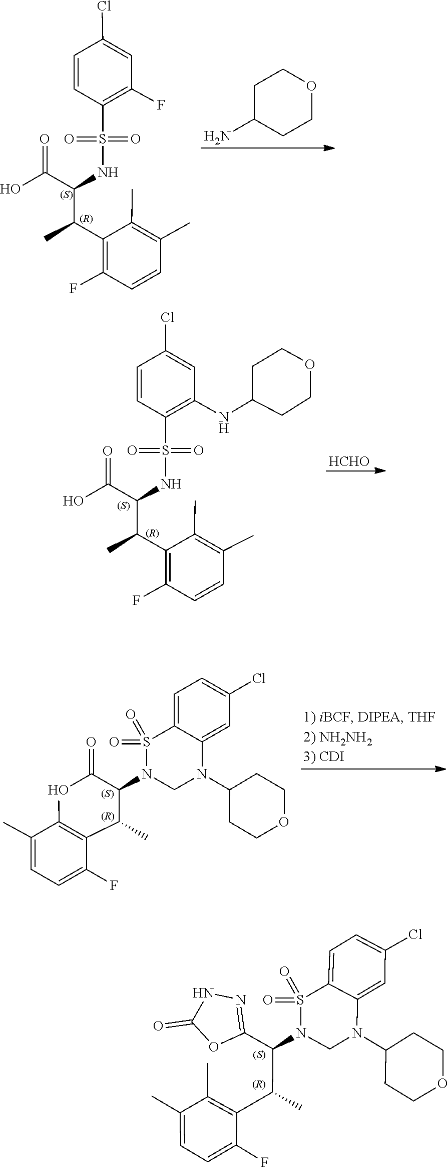

### Step 1: Synthesis of (2S,3R)-2-{4-chloro-2-[(oxan-4-yl)amino]benzenesulfonamido}-3-(6-fluoro-2,3-dimethylphenyl)butanoic acid

The title compound was synthesized following the procedure described for Ex-30 step 2 substituting 3-methoxypropylamine with 4-aminotetrahydropyran. The reaction was carried out at 95° C. for 4 days

### Step 2: Synthesis of (2S,3R)-2-(6-chloro-1,1-dioxido-4-(tetrahydro-2H-pyran-4-yl)-3,4-dihydro-2H-benzo[e][1,2,4]thiadiazin-2-yl)-3-(6-fluoro-2,3-dimethylphenyl)butanoic acid

The title compound was synthesized following the procedure described for Ex-30 substituting (2S,3R)-2-{4-chloro-2-[(3-methoxypropyl)amino]benzenesulfonamido}-3-(6-fluoro-2,3-dimethylphenyl)butanoic acid with (2S,3R)-2-((4-chloro-2-((tetrahydro-2H-pyran-4-yl)amino)phenyl)sulfonamido)-3-(6-fluoro-2,3-dimethylphenyl)butanoic acid.

### Step 3: Synthesis of 5-((1S,2R)-1-(6-chloro-1,1-dioxido-4-(tetrahydro-2H-pyran-4-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-30 substituting 2S,3R)-2-(6-chloro-4-(3-methoxypropyl)-1,1-dioxido-3,4-dihydro-2H-benzo[e][1,2,4]thiadiazin-2-yl)-3-(6-fluoro-2,3-dimethylphenyl) butanoic with (2S,3R)-2-((4-chloro-2-((tetrahydro-2H-pyran-4-yl)amino)phenyl)sulfonamido)-3-(6-fluoro-2,3-dimethylphenyl)butanoic acid yielding the title compound. ¹H NMR (400 MHz, Methanol-d₄) δ 7.56 (d, J=8.4 Hz, 1H), 7.00-6.94 (m, 2H), 6.77 (dd, J=8.4, 1.8 Hz, 1H), 6.70 (dd, J=12.1, 8.4 Hz, 1H), 5.42 (dd, J=11.8, 1.6 Hz, 1H), 5.20 (d, J=1.6 Hz, 2H), 4.12-4.06 (m, 1H), 4.06-3.96 (m, 2H), 3.96-3.88 (m, 1H), 3.58 (dtd, J=20.6, 11.7, 2.1 Hz, 2H), 2.37 (s, 3H), 2.21 (s, 3H), 2.17-2.05 (m, 1H), 1.97-1.84 (m, 2H), 1.82-1.74 (m, 1H), 1.46 (dd, J=6.9, 1.1 Hz, 3H). LC-MS: m/z=549.43 [M−H]−.

### Example 34: 5-((1S,2R)-1-(6-chloro-4-(3-(dimethylamino)propyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

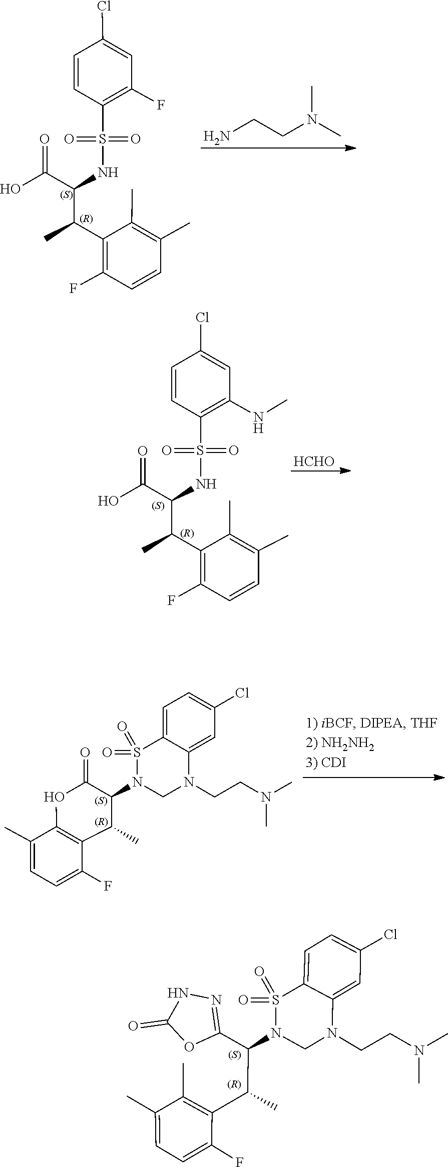

### Step 1: Synthesis of (2S,3R)-2-(4-chloro-2-{[3-(dimethylamino)propyl]amino}benzolsulfonamido)-3-(6-fluoro-2,3-dimethylphenyl)butanoic acid

The title compound was synthesized following the procedure described for Ex-30 substituting 3-methoxypropylamine with N,N-Dimethyl-1,3-propanediamine.

### Step 2: Synthesis of (2S,3R)-2-(6-chloro-4-(3-(dimethylamino)propyl)-1,1-dioxido-3,4-dihydro-2H-benzo[e][1,2,4]thiadiazin-2-yl)-3-(6-fluoro-2,3-dimethylphenyl)butanoic acid

The title compound was synthesized following the procedure described for Ex-30substituting (2S,3R)-2-{4-chloro-2-[(3-methoxypropyl)amino]benzenesulfonamido}-3-(6-fluoro-2,3-dimethylphenyl)butanoic acid with (2S,3R)-2-(4-chloro-2-{[3-(dimethylamino)propyl]amino}benzenesulfonamido)-3-(6-fluoro-2,3-dimethylphenyl)butanoic acid.

### Step 3: Synthesis of 5-((1S,2R)-1-(6-chloro-4-(3-(dimethylamino)propyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-30 substituting ((2S,3R)-2-{4-chloro-2-[(3-methoxypropyl)amino]benzenesulfonamido}-3-(6-fluoro-2,3-dimethylphenyl)butanoic acid with (2S,3R)-2-(6-chloro-4-(3-(dimethylamino)propyl)-1,1-dioxido-3,4-dihydro-2H-benzo[e][1,2,4]thiadiazin-2-yl)-3-(6-fluoro-2,3-dimethylphenyl)butanoic acid yielding the title compound. ¹H NMR (300 MHz, Methanol-d₄) δ 7.56 (d, J=8.3 Hz, 1H), 7.00-6.92 (m, 1H), 6.81-6.74 (m, 2H), 6.69 (dd, J=12.1, 8.4 Hz, 1H), 5.44 (d, J=11.9 Hz, 1H), 5.38 (d, J=14.6 Hz, 1H), 5.10 (d, J=14.4 Hz, 1H), 3.89 (dq, J=13.5, 6.6 Hz, 1H), 3.48 (dt, J=14.7, 7.4 Hz, 1H), 3.20 (dt, J=15.3, 7.8 Hz, 1H), 2.49-2.40 (m, 2H), 2.39 (s, 3H), 2.33 (s, 6H), 2.22 (s, 3H), 1.79 (p, J=7.4 Hz, 2H), 1.45 (d, J=6.9 Hz, 3H). LC-MS: m/z=550.42 [M−H]−

### Example 35: 5-((1S,2R)-1-(7-chloro-4-methyl-1,1-dioxido-5-oxo-4,5-dihydrobenzo[f][1,2,4]thiadiazepin-2 (3H)-yl)-2-(6-fluoro-2,3-dimethylphenyl)propyl)-1,3,4-oxadiazol-2 (3H)-one

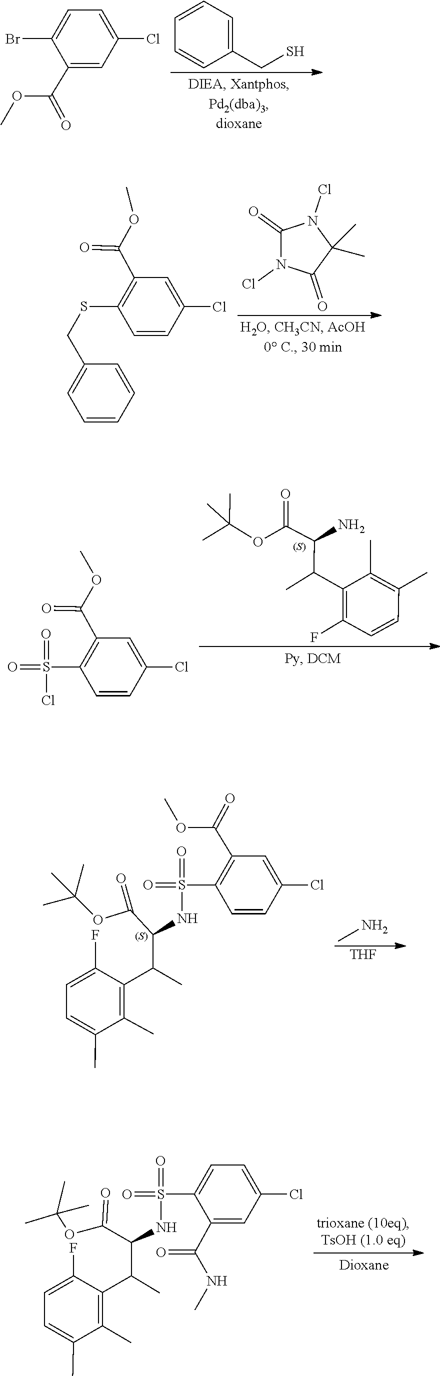

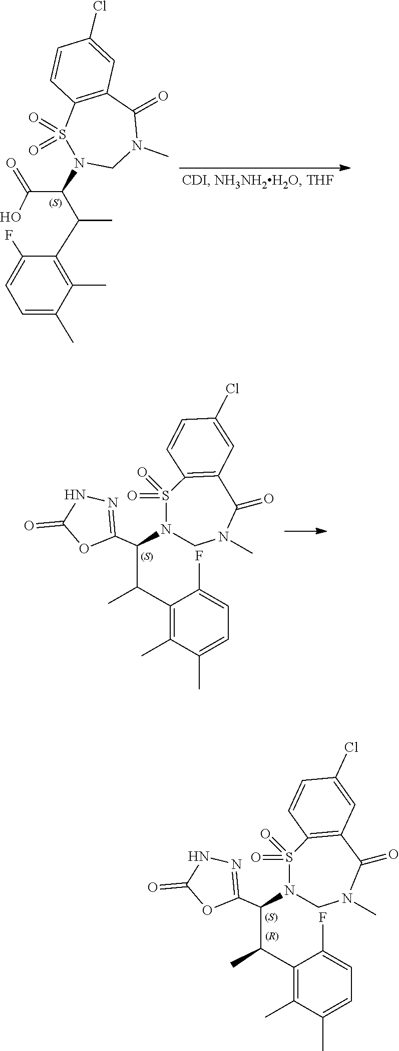

### Step 1: Synthesis of methyl 2-(benzylsulfanyl)-5-chlorobenzoate

Into a 100 mL round-bottom flask were added methyl 2-bromo-5-chlorobenzoate (5 g, 20 mmol, 1 equiv) and Dioxane (50 mL) at room temperature. To the above mixture was added Pd₂(dba)₃ (1835 mg, 2 mmol, 0.1 equiv), XantPhos (2319 mg, 4.008 mmol, 0.2 equiv), DIEA (9.94 mL, 60.123 mmol, 3 equiv) and benzyl mercaptan (2823.15 μL, 24.049 mmol, 1.2 equiv). The resulting mixture was stirred overnight at 100° C. under nitrogen atmosphere. The mixture was allowed to cool down to room temperature. The resulting mixture was extracted with EtOAc (3×20 mL). The combined organic layers were washed with brine (1×100 mL), dried over anhydrous Na₂SO₄. After filtration, the filtrate was concentrated under reduced pressure. The residue was purified by silica gel column chromatography, eluted with PEI/EtOAc (12:1) to afford methyl 2-(benzylsulfanyl)-5-chlorobenzoate (5.5 g, 93.7%).

### Step 2: Synthesis of methyl 5-chloro-2-(chlorosulfonyl)benzoate

Into a 250 mL 3-necked round-bottom flask were added methyl 2-(benzylsulfanyl)-5-chlorobenzoate (5.5 g, 18.8 mmol, 1 equiv) and H₂O (1.6 mL), AcOH (8 mL) in MeCN (55 mL). To the mixture was added 1,3-dichloro-5,5-dimethylimidazolidine-2,4-dione (7.4 g, 37.6 mmol, 2 equiv) dropwise at 0° C. The resulting mixture was stirred for 30 min at 0° C. under nitrogen atmosphere. The reaction was quenched with water. The resulting mixture was extracted with EtOAc. The combined organic layers were washed with brine, dried over anhydrous Na₂SO₄. After filtration, the filtrate was concentrated. This resulted in methyl 5-chloro-2-(chlorosulfonyl) benzoate (5.1 g).

### Step 3: Synthesis of methyl 2-(N-((2S)-1-(tert-butoxy)-3-(6-fluoro-2,3-dimethylphenyl)-1-oxobutan-2-yl) sulfamoyl)-5-chlorobenzoate

To a stirred solution of tert-butyl (2S)-2-amino-3-(6-fluoro-2,3-dimethylphenyl) butanoate (1.31 g, 4.66 mmol, 1.00 equiv) in DCM (10 mL) were added pyridine (1.88 mL, 23.3 mmol, 5 equiv) and methyl 5-chloro-2-(chlorosulfonyl) benzoate (1.63 g, 6.1 mmol, 1.3 equiv) in portions at 0° C., then the mixture was stirred at room temperature overnight. The resulting mixture was concentrated under vacuum. The residue was purified by silica gel column chromatography, eluted with PE/EtOAc (92:8) to afford methyl 2-(N-((2S)-1-(tert-butoxy)-3-(6-fluoro-2,3-dimethylphenyl)-1-oxobutan-2-yl) sulfamoyl)-5-chlorobenzoate (2 g, 83.6%).

### Step 4: Synthesis of tert-butyl (2S)-2-((4-chloro-2-(methylcarbamoyl) phenyl) sulfonamido)-3-(6-fluoro-2,3-dimethylphenyl) butanoate

To a stirred solution of methyl 2-(N-((2S)-1-(tert-butoxy)-3-(6-fluoro-2,3-dimethylphenyl)-1-oxobutan-2-yl) sulfamoyl)-5-chlorobenzoate (100 mg, 0.2 mmol, 1 equiv) in THF was added methylamine (0.49 mL, 0.98 mmol, 5 equiv) at room temperature. The resulting mixture was stirred overnight at 60° C. The resulting mixture was concentrated under vacuum. The residue was purified by silica gel column chromatography, eluted with PE/EtOAc (5:1) to afford tert-butyl (2S)-2-((4-chloro-2-(methylcarbamoyl) phenyl) sulfonamido)-3-(6-fluoro-2,3-dimethylphenyl) butanoate (68 mg, 68.1%).

### Step 5: Synthesis of (2S)-2-(7-chloro-4-methyl-1,1-dioxido-5-oxo-4,5-dihydrobenzo[f][1,2,4]thiadiazepin-2 (3H)-yl)-3-(6-fluoro-2,3-dimethylphenyl)butanoic acid

To a stirred solution of tert-butyl (2S)-2-((4-chloro-2-(methylcarbamoyl) phenyl) sulfonamido)-3-(6-fluoro-2,3-dimethylphenyl) butanoate (10 mg, 0.02 mmol, 1 equiv) and trioxane (263 mg, 2.92 mmol, 10 equiv) in 1,4-dioxane was added TsOH (50 mg, 0.29 mmol, 1 equiv) in portions at room temperature under nitrogen atmosphere. The resulting mixture was stirred overnight at 100° C. under nitrogen atmosphere. The resulting mixture was concentrated under vacuum. The residue was purified by reversed-phase flash chromatography with the following conditions: column, C18 silica gel; mobile phase, MeCN in Water (0.1% FA), 10% to 100% gradient in 10 min; detector, UV 254 nm. This resulted (2S)-2-(7-chloro-4-methyl-1,1-dioxido-5-oxo-4,5-dihydrobenzo[f][1,2,4]thiadiazepin-2 (3H)-yl)-3-(6-fluoro-2,3-dimethylphenyl)butanoic acid (90 mg, 65.6%).

### Step 6: Synthesis of 5-((1S,2R)-1-(7-chloro-4-methyl-1,1-dioxido-5-oxo-4,5-dihydrobenzo[f][1,2,4]thiadiazepin-2 (3H)-yl)-2-(6-fluoro-2,3-dimethylphenyl)propyl)-1,3,4-oxadiazol-2 (3H)-one

To a stirred solution of resulted (2S)-2-(7-chloro-4-methyl-1,1-dioxido-5-oxo-4,5-dihydrobenzo[f][1,2,4]thiadiazepin-2 (3H)-yl)-3-(6-fluoro-2,3-dimethylphenyl)butanoic acid (80 mg, 0.18 mmol, 1 equiv) in THF (1.0 mL) was added CDI (85.6 mg, 0.53 mmol, 3 equiv) in portions at room temperature. The resulting mixture was stirred for 30 min at room temperature. To the above mixture was added hydrazine hydrate (51 μL, 1.06 mmol, 6 equiv) dropwise at 0° C. The resulting mixture was stirred for additional 10 min at 0° C. The reaction was quenched with water/ice at 0° C. The resulting mixture was extracted with EtOAc (2×2 mL). The combined organic layers were washed with brine (1×3 mL), dried over anhydrous Na₂SO₄. After filtration, the filtrate was concentrated under reduced pressure. The residue was dissolved in 1,4-dioxane (0.2 mL). To the above mixture was added CDI (85.6 mg, 0.53 mmol, 3 equiv) in portions at room temperature. The reaction mixture was stirred for additional 1 h at room temperature. The resulting mixture was poured into water and extracted with EtOAc (3×5 mL). The combined organic layers were washed with brine, dried over anhydrous Na₂SO₄ and evaporated. The residue was purified by reversed-phase flash chromatography with the following conditions: column, C18 silica gel; mobile phase, MeCN in Water (10 mmol/L NH₄HCO₃), 10% to 100% gradient in 10 min; detector, UV 254 nm. This resulted in 7-chloro-2-[(1S,2R)-2-(6-fluoro-2,3-dimethylphenyl)-1-(5-oxo-4H-1,3,4-oxadiazol-2-yl) propyl]-4-methyl-3H-1lambda6,2,4-benzothiadiazepine-1,1,5-trione (8.5 mg, 14.17%). LCMS:(ES, m/z): [M−H]: 506.85. ¹H NMR (300 MHz, Methanol-d₄) δ 7.85-7.76 (m, 3H), 7.05-7.00 (dd, J=8.4, 5.7 Hz, 1H), 6.82-6.75 (dd, J=12.2, 8.4 Hz, 1H), 5.61-5.57 (m, 1H), 4.91 (d, J=13.7 Hz, 16H), 4.57-4.51 (d, J=13.7 Hz, 1H), 3.93-3.83 (m, 1H), 3.21-3.18 (s, 3H), 2.28-2.03 (d, J=24.1 Hz, 6), 1.45-1.43 (dd, J=7.0, 1.2 Hz, 3H).

### Example 36: 5-((1S,2R)-1-(7-chloro-6-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

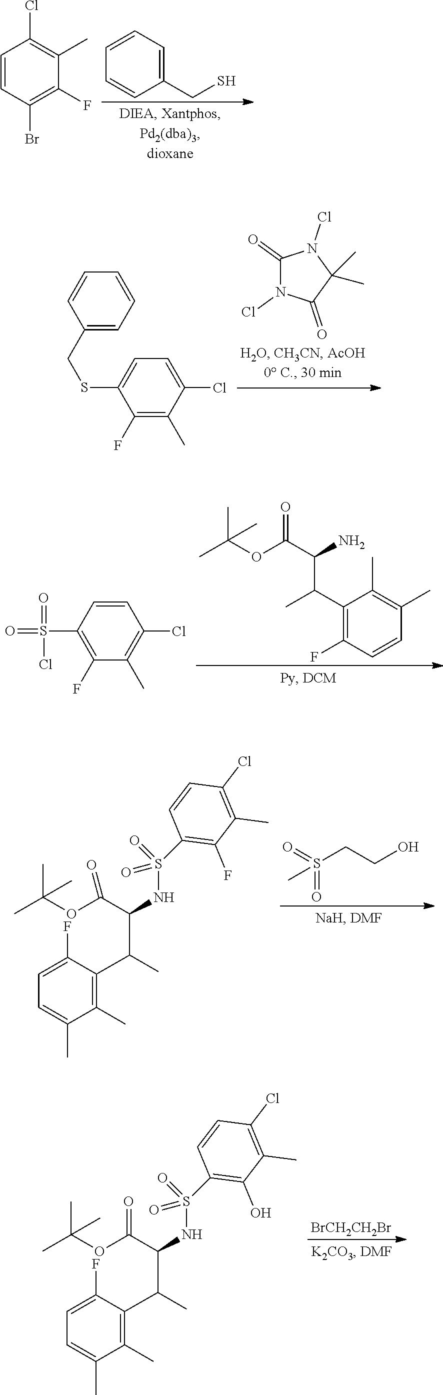

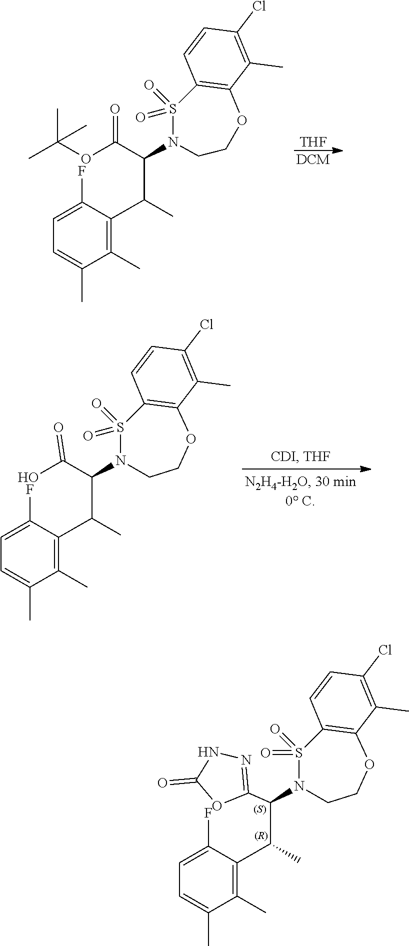

### Step 1: Synthesis of 1-(benzylsulfanyl)-4-chloro-2-fluoro-3-methylbenzene

Into a 40 mL vial were added 1-bromo-4-chloro-2-fluoro-3-methylbenzene (1 g, 4.48 mmol, 1 equiv), dioxane (10 mL), benzyl mercaptan (0.67 g, 5.37 mmol, 1.2 equiv), DIEA (2.3 mL, 13 mmol, 3 equiv), Xantphos (0.26 g, 0.45 mmol, 0.1 equiv) and Pd₂(dba)₃ (0.20 g, 0.22 mmol, 0.05 equiv) at room temperature. The resulting mixture was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc to afford 1-(benzylsulfanyl)-4-chloro-2-fluoro-3-methylbenzene (800 mg, 67%).

### Step 2: Synthesis of 4-chloro-2-fluoro-3-methylbenzenesulfonyl chloride

Into a 20 mL vial were added 1-(benzylsulfanyl)-4-chloro-2-fluoro-3-methylbenzene (800 mg, 3 mmol, 1 equiv), CH₃CN (8 mL), H₂O (0.4 ml), AcOH (560 μL, 9.8 mmol, 3.3 equiv). To the mixture was added 1,3-dichloro-5,5-dimethylimidazolidine-2,4-dione (1182 mg, 6 mmol, 2 equiv) at 0° C. The resulting mixture was stirred 30 min at 0° C. The resulting mixture was quenched with water and then extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc to afford 4-chloro-2-fluoro-3-methylbenzenesulfonyl chloride (700 mg, 96%).

### Step 3: Synthesis of tert-butyl (2S)-2-((4-chloro-2-fluoro-3-methylphenyl) sulfonamido)-3-(6-fluoro-2,3-dimethylphenyl) butanoate

Into a 25 mL round-bottom flask were added tert-butyl (2S)-2-amino-3-(6-fluoro-2,3-dimethylphenyl) butanoate (540 mg, 1.92 mmol, 1 equiv), DCM (7 mL), Pyridine (759 mg, 10 mmol, 5 equiv). To the mixture was added 4-chloro-2-fluoro-3-methylbenzenesulfonyl chloride (700 mg, 2.9 mmol, 1.5 equiv) at 0° C. The resulting mixture was stirred 1 h at room temperature. The resulting mixture was concentrated under reduced pressure. The residue was purified by reverse flash chromatography with the following conditions: column, C18 silica gel; mobile phase, MeCN in Water (0.1% FA), 10% to 50% gradient in 10 min; detector, UV 254 nm. This resulted in tert-butyl (2S)-2-(4-chloro-2-fluoro-3-methylbenzenesulfonamido)-3-(6-fluoro-2,3-dimethylphenyl) butanoate (700 mg, 74.7%).

### Step 4: Synthesis of tert-butyl (2S)-2-(4-chloro-2-hydroxy-3-methylbenzenesulfonamido)-3-(6-fluoro-2,3-dimethylphenyl) butanoate

Into a 20 mL vial were added tert-butyl (2S)-2-(4-chloro-2-fluoro-3-methylbenzenesulfonamido)-3-(6-fluoro-2,3-dimethylphenyl) butanoate (600 mg, 1.23 mmol, 1 equiv), DMF (6 mL), 2-methanesulfonylethanol (229 mg, 1.85 mmol, 1.5 equiv). To the mixture was added NaH 60% (88.5 mg, 3.69 mmol, 3 equiv) at 0° C. The resulting mixture was stirred 5 h at room temperature under nitrogen atmosphere. The resulting mixture was quenched with water and extracted with EtOAc. The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl (2S)-2-(4-chloro-2-hydroxy-3-methylbenzenesulfonamido)-3-(6-fluoro-2,3-dimethylphenyl) butanoate (500 mg, 83.7%).

### Step 5: Synthesis of tert-butyl (2S)-2-(7-chloro-6-methyl-1,1-dioxido-3,4-dihydro-2H-benzo[b][1,4,5]oxathiazepin-2-yl)-3-(6-fluoro-2,3-dimethylphenyl)butanoate

Into a 10 mL sealed tube were added tert-butyl (2S)-2-(4-chloro-2-hydroxy-3-methylbenzenesulfonamido)-3-(6-fluoro-2,3-dimethylphenyl) butanoate (490 mg, 1 mmol, 1 equiv), DMF (5 mL), dibromoethane (290 mg, 1.54 mmol, 1.5 equiv) and Cs₂CO₃ (1 g, 3 mmol, 3 equiv) at room temperature. The resulting mixture was stirred overnight at 60° C. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl (2S)-2-(7-chloro-6-methyl-1,1-dioxido-3,4-dihydro-2H-benzo[b][1,4,5]oxathiazepin-2-yl)-3-(6-fluoro-2,3-dimethylphenyl)butanoate (200 mg, 38%).

### Step 6: Synthesis of (2S)-2-(7-chloro-6-methyl-1,1-dioxido-3,4-dihydro-2H-benzo[b][1,4,5]oxathiazepin-2-yl)-3-(6-fluoro-2,3-dimethylphenyl)butanoic acid

Into a 20 mL vial were added tert-butyl (2S)-2-(7-chloro-6-methyl-1,1-dioxido-3,4-dihydro-2H-benzo[b][1,4,5] oxathiazepin-2-yl)-3-(6-fluoro-2,3-dimethylphenyl)butanoate (200 mg, 0.39 mmol, 1 equiv), DCM (3 mL) and TFA (1 mL) at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2S)-2-(7-chloro-6-methyl-1,1-dioxido-3,4-dihydro-2H-benzo[b][1,4,5]oxathiazepin-2-yl)-3-(6-fluoro-2,3-dimethylphenyl)butanoic acid (150 mg, 84.23%).

### Step 7: Synthesis of 5-((1S,2R)-1-(7-chloro-6-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 8 mL vial were (2S)-2-(7-chloro-6-methyl-1,1-dioxido-3,4-dihydro-2H-benzo[b][1,4,5]oxathiazepin-2-yl)-3-(6-fluoro-2,3-dimethylphenyl)butanoic acid (50 mg, 0.11 mmol, 1 equiv), THF (0.5 mL) and CDI (46.2 mg, 0.29 mmol, 2.6 equiv) at room temperature. The resulting mixture was stirred for 30 min at room temperature. To the above mixture was added hydrazine hydrate (54.9 mg, 1.1 mmol, 10 equiv) dropwise at 0° C. The resulting mixture was stirred for additional 30 min at 0° C. The resulting mixture was quenched with water and extracted with EtOAc. The combined organic layers were washed with brine, dried over anhydrous Na₂SO₄. After filtration, the filtrate was concentrated under reduced pressure. The residue was dissolved in 1,4-dioxane (0.2 mL). To the above mixture was added CDI (46.2 mg, 0.29 mmol, 2.6 equiv) in portions at room temperature. The reaction mixture was stirred for additional 1 h at room temperature. The resulting mixture was poured into water and extracted with EtOAc (3×5 mL). The combined organic layers were washed with brine, dried over anhydrous Na₂SO₄ and evaporated. The crude product was purified by Chiral-Prep-HPLC with the following conditions: Column, XBridge Prep OBD C18 Column, 19*250 mm, 5 μm; mobile phase, 10 mmol NH₄HCO₃+0.05% NH₃H₂O and MeOH— (50% MeOH— up to 80% in 8 min); Detector, uv 254 nm. This resulted 5-((1S,2R)-1-(7-chloro-6-methyl-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (25.2 mg, 45.6%). LCMS:(ES, m/z): M+H=496.20. ¹H NMR (300 MHz, DMSO-d₆) δ 12.15 (s, 1H), 7.59 (d, J=8.6 Hz, 1H), 7.36 (d, J=8.6 Hz, 1H), 7.03 (dd, J=8.4, 5.9 Hz, 1H), 6.83 (dd, J=12.2, 8.4 Hz, 1H), 5.40 (dd, J=11.6, 2.0 Hz, 1H), 4.59-4.48 (m, 1H), 3.98-3.81 (m, 4H), 2.28 (m, 3H), 2.22 (m, 3H), 2.17 (m, 3H), 1.32 (d, J=6.8 Hz, 3H).

### Example 37 and 39: 6-chloro-2-((1S,2R)-2-(6-fluoro-2,3-dimethylphenyl)-1-(5-oxo-4,5-dihydro-1,3,4-oxadiazol-2-yl) propyl)-N,N,4-trimethyl-3,4-dihydro-2H-benzo[e][1,2,4]thiadiazine-8-carboxamide 1,1-dioxide

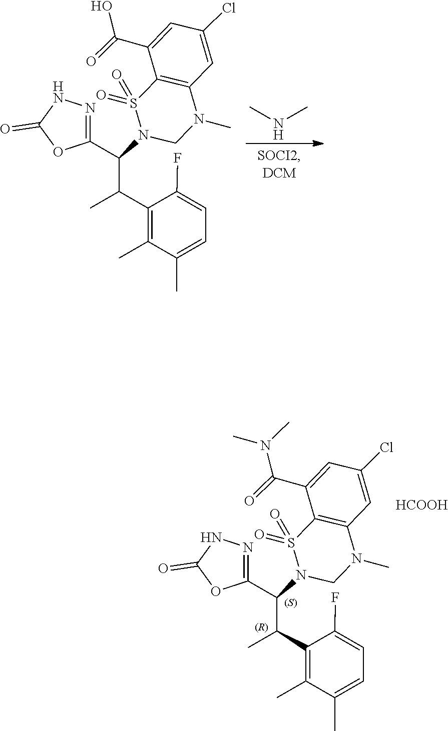

SOCl₂ (600 μL) is added to 6-chloro-2-((1S)-2-(6-fluoro-2,3-dimethylphenyl)-1-(5-oxo-4,5-dihydro-1,3,4-oxadiazol-2-yl)propyl)-4-methyl-3,4-dihydro-2H-benzo[e][1,2,4]thiadiazine-8-carboxylic acid 1,1-dioxide (see example 22)(60 mg, 0.11 mmol, 1 equiv) at room temperature. The resulting mixture was stirred for 1 h at 80° C. The resulting mixture was concentrated under reduced pressure. The residue was dissolved in DCM (0.3 mL). To the above mixture was added dimethylamine (300 μL, 6.65 mmol, 58.2 equiv) dropwise at 0° C. The resulting mixture was stirred for additional 1 h at room temperature. The resulting mixture was concentrated under reduced pressure. The residue was purified by reversed-phase flash chromatography using the following conditions: Column: XBridge Prep Phenyl OBD Column, 19*250 mm, 5 μm; Mobile Phase A: Water (0.1% FA), Mobile Phase B: MeOH-HPLC; Flow rate: 20 mL/min; Gradient: 20% B to 50% B in 8 min, 50% B; Wave Length: 254 nm; RT1 (min): 7.5. This resulted in 6-chloro-2-((1S,2R)-2-(6-fluoro-2,3-dimethylphenyl)-1-(5-oxo-4,5-dihydro-1,3,4-oxadiazol-2-yl) propyl)-N,N,4-trimethyl-3,4-dihydro-2H-benzo[e][1,2,4]thiadiazine-8-carboxamide 1,1-dioxide (11.7 mg, 16.6%). LC-MS: (ES, m/z): [M+H]+=552.15. ¹H NMR (300 MHz, Methanol-d₄) δ 6.99-6.97 (dd, J=8.5, 5.8 Hz, 1H), 6.74-6.70 (m, 2H), 6.62-6.61 (m, 1H), 5.45-5.40 (m, 2H), 5.09-5.04 (d, J=14.5 Hz, 1H), 4.60-4.58 (s, 1H), 4.01-3.89 (dq, J=13.3, 7.0, 6.6 Hz, 1H), 3.09-2.96 (s, 3H), 2.92-2.84 (d, J=10.9 Hz, 6H), 2.37-2.34 (s, 3H), 2.34-2.21 (s, 3H), 1.49-1.44 (m, 3H).

The second isomer 6-chloro-2-((1S,2R)-2-(6-fluoro-2,3-dimethylphenyl)-1-(5-oxo-4,5-dihydro-1,3,4-oxadiazol-2-yl)propyl)-N,N,4-trimethyl-3,4-dihydro-2H-benzo[e][1,2,4]thiadiazine-8-carboxamide 1,1-dioxide was isolated. [M+H]+=552.15. ¹H NMR (300 MHz, Methanol-d₄) δ 7.18-7.04 (m, 1H), 6.95-6.78 (m, 1H), 6.76-6.75 (m, 1H), 6.72-6.60 (d, J=1.9 Hz, 1H), 5.40-5.30 (d, J=11.5 Hz, 1H), 5.15-5.05 (d, J=14.3 Hz, 1H), 4.55-4.65 (m, 1H), 4.1-3.90 (dd, J=12.0, 6.8 Hz, 1H), 3.06-2.99 (s, 3H), 3.02-2.85 (m, 6H), 2.40-2.35 (d, J=11.5 Hz, 3H), 2.30-2.29 (s, 3H), 1.28-1.26 (d, J=7.1 Hz, 3H)

### Example 38:5-((1S,2R)-1-(6-chloro-4-(2-(dimethylamino)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

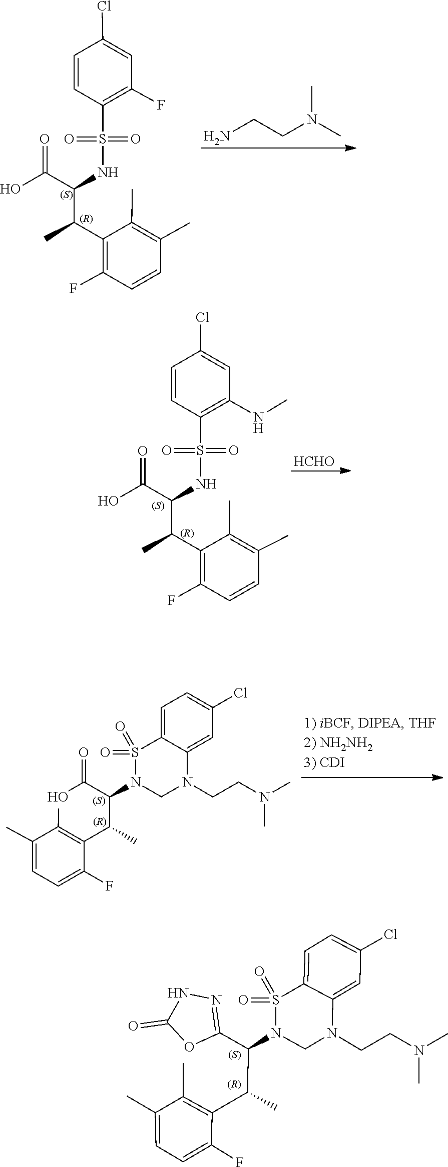

### Synthesis of (2S,3R)-2-((4-chloro-2-((2-(dimethylamino)ethyl)amino)phenyl)sulfonamido)-3-(6-fluoro-2,3-dimethylphenyl)butanoic acid

The title compound was synthesized following the procedure described for Ex-4 substituting 3-methoxypropylamine with N,N-dimethylethylenediamine yielding the title compound. LCMS:(ES, m/z): M+H=538.18. ¹H NMR (400 MHz, Methanol-d₄) δ 7.57 (d, J=9.4 Hz, 1H), 6.98 (dd, J=8.4, 5.7 Hz, 1H), 6.78 (dd, J=8.4, 1.8 Hz, 1H), 6.74 (d, J=1.8 Hz, 1H), 6.70 (dd, J=12.1, 8.4 Hz, 1H), 5.47-5.41 (m, 2H), 5.11 (d, J=14.5 Hz, 1H), 3.91 (dq, J=13.5, 6.9 Hz, 1H), 3.52 (ddd, J=13.9, 8.7, 4.5 Hz, 1H), 3.37-3.25 (m, 1H), 2.60-2.50 (m, 1H), 2.46 (ddd, J=12.8, 8.6, 4.6 Hz, 1H), 2.39 (s, 3H), 2.32 (s, 6H), 2.22 (s, 3H), 1.45 (dd, J=6.9, 1.1 Hz, 3H.

### Example 40: 5-((1S)-1-(7-chloro-6-(2-hydroxypropan-2-yl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

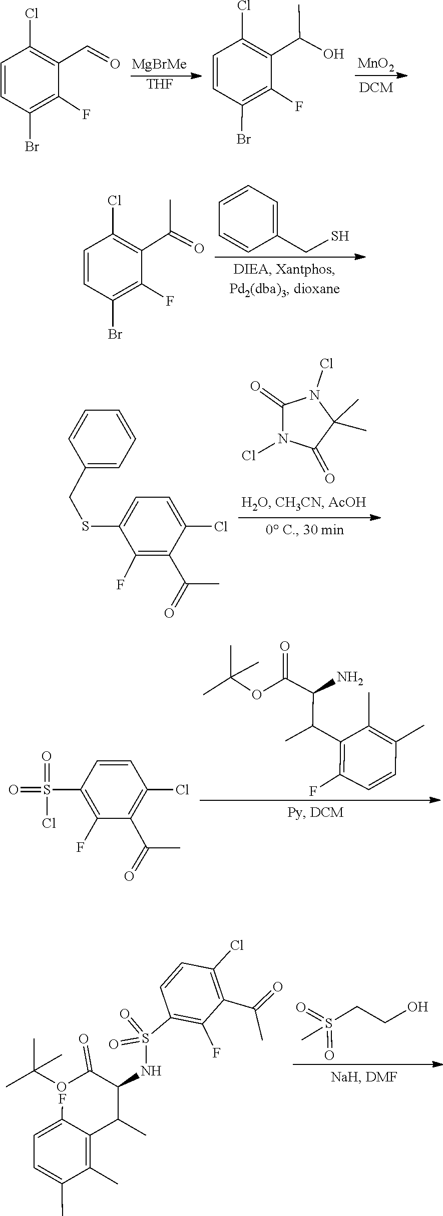

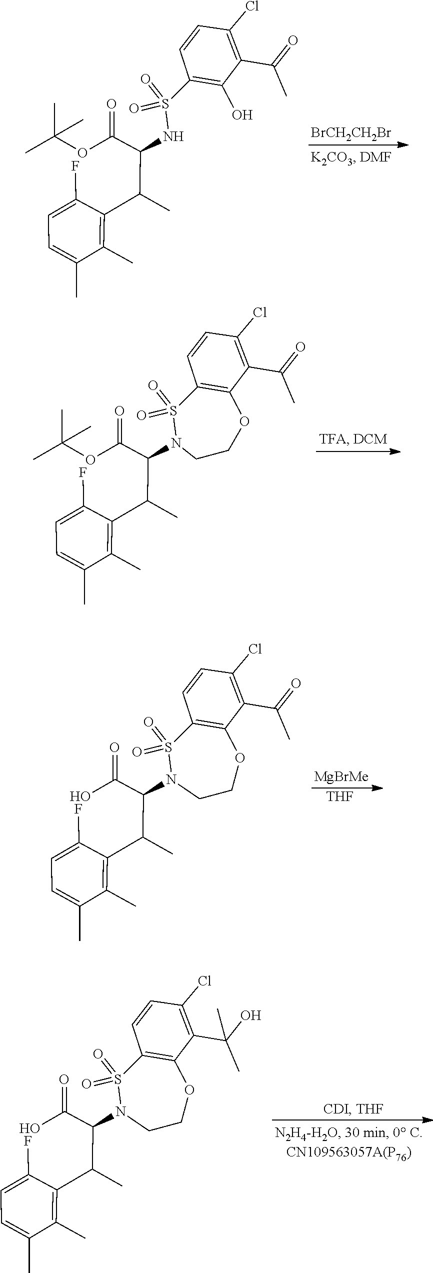

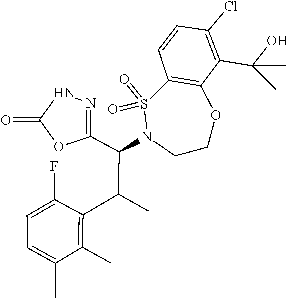 [SMILES: Cc1ccc(F)c(C(C)[C@@H](c2n[nH]c(=O)o2)N2CCOc3c(ccc(Cl)c3C(C)(C)O)S2(=O)=O)c1C]

### Step 1: Synthesis of 1-(3-bromo-6-chloro-2-fluorophenyl) ethan-1-ol

To a stirred solution of 3-bromo-6-chloro-2-fluorobenzaldehyde (5 g, 21.1 mmol, 1 equiv) in THF was added CH3MgBr (3M in Et2O) (18 mL, 52.6 mmol, 2.5 equiv) dropwise at 0° C. under nitrogen atmosphere. The resulting mixture was stirred for 30 min at room temperature under nitrogen atmosphere.

The reaction was quenched with sat. NH₄Cl (aq.) at room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The crude product was directly used in the next step without further purification.

### Step 2: Synthesis of 1-(3-bromo-6-chloro-2-fluorophenyl) ethenone

Into a 250 mL round-bottom flask were added 1-(3-bromo-6-chloro-2-fluorophenyl) ethanol (5.1 g, 20.1 mmol, 1 equiv), DCM (100 mL) and MnO2 (17.6 g, 201 mmol, 10 equiv) at room temperature. The resulting mixture was stirred overnight at 40 TC. The resulting mixture was filtered, the filter cake was washed with CH₂Cl₂ (3×50 mL). The filtrate was concentrated under reduced pressure. The residue was purified by silica gel column chromatography, eluted with PE to afford 1-(3-bromo-6-chloro-2-fluorophenyl) ethanone (3.95 g, 78.1%) as a light-yellow oil.

### Step 3: Synthesis of 1-[3-(benzylsulfanyl)-6-chloro-2-fluorophenyl]ethenone

Into a 100 mL round-bottom flask were added 1-(3-bromo-6-chloro-2-fluorophenyl) ethanone (2 g, 8 mmol, 1 equiv), dioxane (35 mL), benzyl mercaptan (1.13 mL, 9.5 mmol, 1.2 equiv), DIEA (4.2 mL, 24 mmol, 3 equiv), Xantphos (920 mg, 1.6 mmol, 0.2 equiv) and Pd₂(dba)₃ (728 mg, 0.8 mmol, 0.1 equiv) at room temperature. The resulting mixture was stirred overnight at 110° C. under nitrogen atmosphere. The resulting mixture was filtered, the filter cake was washed with CH₂Cl₂ (3×10 mL). The filtrate was concentrated under reduced pressure. The residue was purified by silica gel column chromatography, eluted with PE/EtOAc (10:1) to afford 1-[3-(benzylsulfanyl)-6-chloro-2-fluorophenyl]ethanone (2.44 g, 93.7%) as a light-yellow solid.

### Step 4: Synthesis of -acetyl-4-chloro-2-fluorobenzenesulfonyl chloride

To a stirred solution of 1-[3-(benzylsulfanyl)-6-chloro-2-fluorophenyl]ethanone (2.44 g, 8.3 mmol, 1 equiv) and H₂O (0.8 mL), AcOH (4 mL) in MeCN (20 mL) was added 1,3-dichloro-5,5-dimethylimidazolidine-2,4-dione (3.3 g, 16.6 mmol, 2 equiv) dropwise at 0° C. The resulting mixture was stirred for 30 min at 0° C. The reaction was quenched with sat. NH₄Cl (aq.) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (8:1) to afford 3-acetyl-4-chloro-2-fluorobenzenesulfonyl chloride (1.8 g, 80.2%).

### Step 5: Synthesis of tert-butyl (2S)-2-(3-acetyl-4-chloro-2-fluorobenzenesulfonamido)-3-(6-fluoro-2,3-dimethylphenyl) butanoate

Into a 100 mL round-bottom flask were added tert-butyl (2S)-2-amino-3-(6-fluoro-2,3-dimethylphenyl) butanoate (1.70 g, 6.0 mmol, 1 equiv), DCM (15 mL) and Pyridine (2.44 mL, 30.1 mmol, 5 equiv) at 0° C. To the above mixture was added 3-acetyl-4-chloro-2-fluorobenzenesulfonyl chloride (1.8 g, 6.64 mmol, 1.1 equiv) in DCM (15 mL) at 0° C. The resulting mixture was stirred overnight at room temperature. The resulting mixture was washed with 1×20 mL of water. The resulting mixture was extracted with CH₂Cl₂ (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EA (7:1) to afford tert-butyl (2S)-2-(3-acetyl-4-chloro-2-fluorobenzenesulfonamido)-3-(6-fluoro-2,3-dimethylphenyl) butanoate (2.58 g, 82.83%) as a light-yellow solid.

### Step 6: Synthesis of tert-butyl (2S)-2-((3-acetyl-4-chloro-2-hydroxyphenyl) sulfonamido)-3-(6-fluoro-2,3-dimethylphenyl) butanoate

To a solution of tert-butyl (2S)-2-(3-acetyl-4-chloro-2-fluorobenzenesulfonamido)-3-(6-fluoro-2,3-dimethylphenyl) butanoate (1 g, 1.94 mmol, 1 equiv) in DMF was added sodium hydride (60% in oil, 310 mg) at 0° C. The mixture was stirred for 15 min. 2-methanesulfonylethanol (481 mg, 3.88 mmol, 2 equiv) was added and the mixture was allowed to warm to RT and stirred 2 h at room temperature under nitrogen atmosphere. The reaction was quenched with sat. NH₄Cl (aq.) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (1:1) to afford tert-butyl(2S)-2-(3-acetyl-4-chloro-2-hydroxybenzenesulfonamido)-3-(6-fluoro-2,3-dimethylphenyl) butanoate (410 mg, 41.16%) as a brown semi-solid.

### Step 7: Synthesis of tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate

Into a 40 mL sealed tube were added tert-butyl (2S)-2-(3-acetyl-4-chloro-2-hydroxybenzenesulfonamido)-3-(6-fluoro-2,3-dimethylphenyl) butanoate (400 mg, 0.78 mmol, 1 equiv), DMF (8 mL), dibromoethane (100 μL, 1.17 mmol, 1.5 equiv) and K₂CO₃ (323 mg, 2.33 mmol, 3 equiv) at room temperature. The resulting mixture was stirred overnight at 65° C. The reaction was quenched with sat. NH₄Cl (aq.) at room temperature. The resulting mixture was extracted with EtOAc (3×10 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (10:1) to afford tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate (300 mg, 71.4%).

### Step 8: Synthesis of (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoic acid

Into a 20 mL sealed tube were added tert-butyl (2S)-2-(6-acetyl-7-chloro-1,1-dioxo-3,4-dihydro-5,1lambda6,2-benzoxathiazepin-2-yl)-3-(6-fluoro-2,3-dimethylphenyl) butanoate (420 mg, 0.78 mmol, 1 equiv), DCM (3 mL) and TFA (3 mL) at room temperature. The resulting mixture was stirred for 1 h at room temperature under air atmosphere. The resulting mixture was concentrated under vacuum. The crude product was used in the next step directly without further purification.

### Step 9: Synthesis of (2S)-2-(7-chloro-6-(2-hydroxypropan-2-yl)-1,1-dioxido-3,4-dihydro-2H-benzo[b][1,4,5]oxathiazepin-2-yl)-3-(6-fluoro-2,3-dimethylphenyl)butanoic acid

To a stirred solution of (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoic acid (350 mg, 0.72 mmol, 1 equiv) in THF was added bromo(methyl)magnesium (1.21 mL, 3.62 mmol, 5 equiv) dropwise at 0° C. under nitrogen atmosphere. The resulting mixture was stirred for 3 h at room temperature under nitrogen atmosphere. The residue was purified by silica gel column chromatography, eluted with PE/EtOAc (10:1) to afford (2S)-2-(7-chloro-6-(2-hydroxypropan-2-yl)-1,1-dioxido-3,4-dihydro-2H-benzo[b][1,4,5]oxathiazepin-2-yl)-3-(6-fluoro-2,3-dimethylphenyl)butanoic acid (40 mg, 11.06%).

### Step 10: Synthesis of 5-((1S)-1-(7-chloro-6-(2-hydroxypropan-2-yl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

A solution of 2S)-2-(7-chloro-6-(2-hydroxypropan-2-yl)-1,1-dioxido-3,4-dihydro-2H-benzo[b][1,4,5]oxathiazepin-2-yl)-3-(6-fluoro-2,3-dimethylphenyl)butanoic acid (40 mg, 0.08 mmol, 1 equiv) and CDI (38.9 mg, 0.24 mmol, 3 equiv) in THF was stirred for 20 min at room temperature under air atmosphere. To the above mixture was added hydrazine hydrate (23.3 μL, 0.48 mmol, 6 equiv) dropwise at 0° C. The resulting mixture was stirred for additional 30 min at 0° C. The reaction was quenched with water at room temperature. The resulting mixture was extracted with EtOAc (3×1 mL). The combined organic layers were washed with brine (3×1 mL), dried over anhydrous Na₂SO₄. After filtration, the filtrate was concentrated under reduced pressure. Into an 8 mL sealed tube were added CDI (32.43 mg, 0.200 mmol, 2.5 equiv), dioxane (0.6 mL) and the above mixture in at room temperature. The resulting mixture was stirred for 30 min at room temperature under air atmosphere. The crude product was purified by Chiral-Prep-HPLC with the following conditions: Column: XselectCSH C18 OBDColumn 30*150 mm 5 μm, n; Mobile Phase A: Water (0.1% FA), Mobile Phase B: ACN; Flow rate: 60 mL/min; Gradient: 50% B to 64% B in 12 min, 64% B; Wave Length: 220/254 nm; RT1 (min): 7.08 to afford 5-((1S)-1-(7-chloro-6-(2-hydroxypropan-2-yl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (6.3 mg, 14.20%). Mixture of S,R and S,S diastereoisomers (70/30). LCMS: (ES, m/z): [M−H]+=538.05. ¹H NMR (400 MHz, DMSO-d₆) δ 8.38 (s, 1H), 7.62-7.59 (d, J=8.6 Hz, 1H), 7.33-7.31 (d, J=8.5 Hz, 1H), 7.01-6.98 (dd, J=8.4, 5.8 Hz, 1H), 6.83-6.77 (dd, J=12.2, 8.4 Hz, 1H), 5.40-5.37 (d, J=11.5 Hz, 1H), 5.15 (s, 1H), 4.40 (s, 1H), 3.97 (s, 1H), 3.79-3.72 (dd, J=17.4, 11.1 Hz, 2H), 2.33-2.32 (m, 2H), 2.32-2.22 (s, 3H), 2.19-2.15 (s, 3H), 1.59-1.55 (d, J=15.9 Hz, 6H), 1.33-1.29 (d, J=6.8

### Example 41: 6-chloro-2-((1S,2R)-2-(6-fluoro-2,3-dimethylphenyl)-1-(5-oxo-4,5-dihydro-1,3,4-oxadiazol-2-yl)propyl)-N,N-dimethyl-2,3-dihydrobenzo[e][1,4,3]oxathiazine-8-carboxamide 1,1-dioxide

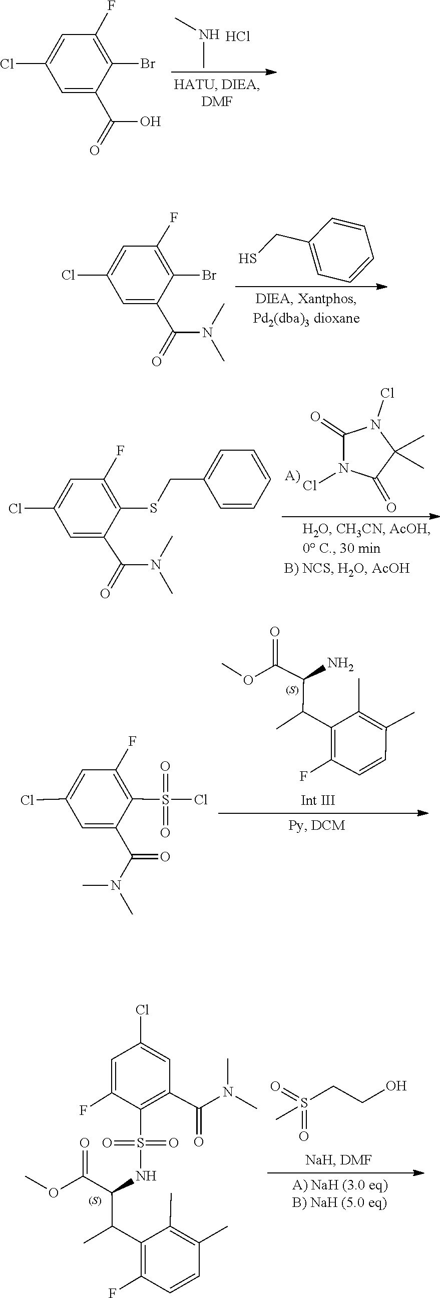

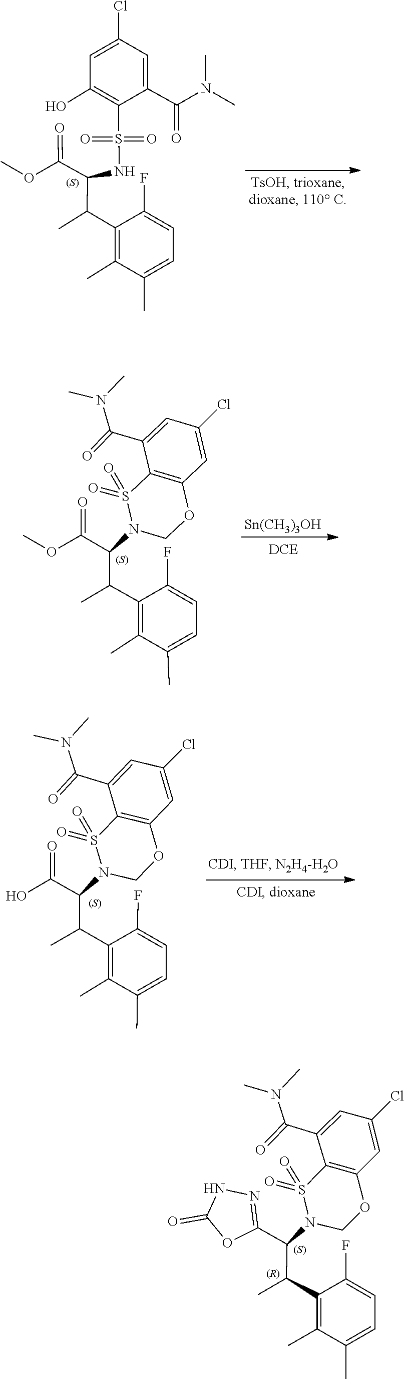

### Step 1: Synthesis of 2-bromo-5-chloro-3-fluoro-N,N-dimethylbenzamide

To a stirred solution of 2-bromo-5-chloro-3-fluorobenzoic acid (3 g, 11.8 mmol, 1 equiv) and dimethylamine hydrochloride (1.16 g, 14.2 mmol, 1.2 equiv) HATU (6.75 g, 17.7 mmol, 1.5 equiv) in DMF was added HATU (6.75 g, 17.7 mmol, 1.5 equiv). The resulting mixture was stirred for 30 min at room temperature. The reaction was quenched by the addition of water (20 mL) at room temperature. The resulting mixture was extracted with EtOAc (2×50 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (5:1) to afford 2-bromo-5-chloro-3-fluoro-N,N-dimethylbenzamide (2.43 g, 73.2%).

### Step 2: Synthesis of 2-(benzylthio)-5-chloro-3-fluoro-N,N-dimethylbenzamide

To a stirred solution of 2-bromo-3-fluoro-N, N-dimethylbenzamide (2.43 g, 9.9 mmol, 1 equiv) and DIEA (5.16 mL, 29.6 mmol, 3 equiv) in 1,4-dioxane was added Pd₂(dba)₃ (904 mg, 1 mmol, 0.1 equiv) and Xantphos (1142 mg, 1.98 mmol, 0.2 equiv) at room temperature. The resulting mixture was stirred for overnight at 100° C. under nitrogen atmosphere. The reaction was quenched with water at room temperature.

The resulting mixture was extracted with EtOAc (2×100 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (6:1) to afford 2-(benzylthio)-5-chloro-3-fluoro-N,N-dimethylbenzamide (2.35 g, 73.5%) as a brown yellow oil.

### Step 3: Synthesis of 4-chloro-2-(dimethylcarbamoyl)-6-fluorobenzenesulfonyl chloride

To a stirred solution of 2-(benzylthio)-5-chloro-3-fluoro-N,N-dimethylbenzamide (2.35 g, 7.26 mmol, 1 equiv), H₂O (1.2 mL), AcOH (4 mL) was added 1,3-dichloro-5,5-dimethylimidazolidine-2,4-dione (2.86 g, 14.5 mmol, 2 equiv) dropwise/in portions at 0° C. The resulting mixture was stirred for 30 min at 0° C. The reaction was quenched with water/Ice at room temperature. The resulting mixture was extracted with EtOAc (2×30 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (6:1) to afford 4-chloro-2-(dimethylcarbamoyl)-6-fluorobenzenesulfonyl chloride (2 g, 91.8%).

### Step 4: Synthesis of methyl (2S)-2-((4-chloro-2-(dimethylcarbamoyl)-6-fluorophenyl)sulfonamido)-3-(6-fluoro-2,3-dimethylphenyl)butanoate

To a stirred solution of methyl (2S)-2-amino-3-(6-fluoro-2,3-dimethylphenyl)butanoate(Int III) (283 mg, 1.18 mmol, 1 equiv) and Pyridine (2.69 mL, 33.3 mmol, 5 equiv) in DCM was added 4-chloro-2-(dimethylcarbamoyl)-6-fluorobenzenesulfonyl chloride (2 g, 6.7 mmol, 1 equiv) in portions at 0° C. The resulting mixture was stirred for overnight at room temperature. The resulting mixture was washed with 1×15 mL of citric acid. The resulting mixture was washed with 2×50 mL of brine. The residue was purified by silica gel column chromatography, eluted with PE/EtOAc (3:1) to afford methyl (2S)-2-[4-chloro-2-(dimethylcarbamoyl)-6-fluorobenzenesulfonamido]-3-(6-fluoro-2,3-dimethylphenyl)butanoate (2 g, 59.7%).

### Step 5: Synthesis of methyl (2S)-2-((4-chloro-2-(dimethylcarbamoyl)-6-hydroxyphenyl)sulfonamido)-3-(6-fluoro-2,3-dimethylphenyl)butanoate

To a stirred solution of methyl (2S)-2-[4-chloro-2-(dimethylcarbamoyl)-6-fluorobenzenesulfonamido]-3-(6-fluoro-2,3-dimethylphenyl)butanoate (2 g, 4 mmol, 1 equiv) and 2-methanesulfonylethanol (987 mg, 8 mmol, 2 equiv) in DMF was added NaH (477 mg, 12 mmol, 3 equiv, 60%) dropwise at 0° C. The resulting mixture was stirred for 2 h at room temperature under nitrogen atmosphere. The reaction was quenched with sat. NH₄Cl (aq.) at room temperature. The resulting mixture was extracted with EtOAc (2×50 mL). The combined organic layers were washed with brine (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 0% to 100% gradient in 20 min; detector, UV 220 nm. This resulted in methyl (2S)-2-[4-chloro-2-(dimethylcarbamoyl)-6-hydroxybenzenesulfonamido]-3-(6-fluoro-2,3-dimethylphenyl)butanoate (780 mg, 39.15%).

### Step 6: Synthesis of methyl (2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoate

To a stirred solution of methyl (2S)-2-[4-chloro-2-(dimethylcarbamoyl)-6-hydroxybenzenesulfonamido]-3-(6-fluoro-2,3-dimethylphenyl)butanoate (300 mg, 0.6 mmol, 1 equiv) and TsOH (103 mg, 0.6 mmol, 1 equiv) in 1,4-dioxane was added 1,3,5-trioxane (539 mg, 6 mmol, 10 equiv) at room temperature. The resulting mixture was stirred for overnight at 110° C. The resulting mixture was concentrated under reduced pressure. The residue was purified by reversed-phase flash chromatography with the following conditions: column, C18 silica gel; mobile phase, MeCN in water (0.1% FA), 0% to 100% gradient in 20 min; detector, UV 220 nm. This resulted in methyl (2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoate (190 mg, 61.9%)

### Step 7: Synthesis of (2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoic acid

To a stirred solution of methyl (2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoate (170 mg, 0.33 mmol, 1 equiv) in DCE (2 mL) was added trimethyltin hydroxide (599 mg, 3.3 mmol, 10 equiv) at room temperature. The resulting mixture was stirred for 5 h at 65° C. The resulting mixture was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 220 nm. This resulted in (2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoic acid (140 mg, 84.7%).

### Step 8: Synthesis of 6-chloro-2-((1S,2R)-2-(6-fluoro-2,3-dimethylphenyl)-1-(5-oxo-4,5-dihydro-1,3,4-oxadiazol-2-yl)propyl)-N,N-dimethyl-2,3-dihydrobenzo[e][1,4,3]oxathiazine-8-carboxamide 1,1-dioxide

To a stirred solution of 2S)-2-(6-chloro-8-(dimethylcarbamoyl)-1,1-dioxidobenzo[e][1,4,3]oxathiazin-2 (3H)-yl)-3-(6-fluoro-2,3-dimethylphenyl)butanoic acid (110 mg, 0.22 mmol, 1 equiv) and CDI (89.4 mg, 0.55 mmol, 2.5 equiv) in THF in portions at room temperature. The resulting mixture was stirred for 90 min at room temperature. To the above mixture was added NH₂NH₂·H₂O (54 μL, 1.1 mmol, 5 equiv) dropwise at 0° C. The resulting mixture was stirred for additional 30 min at 0° C. The reaction was quenched with water/ice at room temperature. The resulting mixture was extracted with EtOAc (2×30 mL). The combined organic layers were washed with brine (2×50 mL), dried over anhydrous Na₂SO₄. After filtration, the filtrate was concentrated under reduced pressure. To the above mixture was added CDI (89.4 mg, 0.55 mmol, 2.5 equiv) in dioxane (2 ml) dropwise at room temperature. The resulting mixture was stirred for additional 1 h at room temperature. The reaction was quenched with water at room temperature. The resulting mixture was concentrated under reduced pressure. The residue was purified by reversed-phase flash chromatography with the following conditions: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 55% B in 8 min, 55% B; Wave Length: 254 nm; RT1 (min): 7. This resulted in 6-chloro-2-((1S,2R)-2-(6-fluoro-2,3-dimethylphenyl)-1-(5-oxo-4,5-dihydro-1,3,4-oxadiazol-2-yl)propyl)-N,N-dimethyl-2,3-dihydrobenzo[e][1,4,3]oxathiazine-8-carboxamide 1,1-dioxide (42.9 mg, 35.3%). LCMS:(ES, m/z): [M+H]+=539.15. ¹H NMR (300 MHz, Methanol-d₄) δ 7.07-6.98 (m, 3H), 6.80-6.70 (dd, J=12.2, 8.4 Hz, 1H), 6.15-5.84 (m, 2H), 5.60-5.50 (d, J=11.8 Hz, 1H), 4.60-4.50 (s, 1H), 4.10-3.90 (dd, J=11.7, 7.0 Hz, 1H), 3.20-3.00 (s, 3H), 2.94-2.84 (s, 3H), 2.38-2.30 (s, 3H), 2.29-2.15 (s, 3H), 1.45-1.35 (m, 3H).

### Example 42: 5-((1S,2R)-1-(6-chloro-4-(oxetan-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using 3-aminooxetane(neat) instead of 3-methoxypropylamine to obtain 5-((1S,2R)-1-(6-chloro-4-(oxetan-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.009 g, yield 26%). LCMS:(ES, m/z): [M−H]−=521.10. ¹H NMR (400 MHz, DMSO-d₆) δ 12.13 (s, 1H), 7.64 (d, J=8.5 Hz, 1H), 7.02 (dd, J=8.4, 5.8 Hz, 1H), 6.91 (dd, J=8.4, 1.8 Hz, 1H), 6.82 (dd, J=12.3, 8.3 Hz, 1H), 6.69 (d, J=1.9 Hz, 1H), 5.46 (d, J=14.9 Hz, 1H), 5.34-5.27 (m, 2H), 5.01 (t, J=6.7 Hz, 1H), 4.87 (p, J=7.3 Hz, 1H), 4.79-4.71 (m, 2H), 4.68 (t, J=6.8 Hz, 1H), 4.08-3.96 (m, 1H), 2.32 (s, 3H), 2.17 (s, 3H), 1.35 (d, J=6.7 Hz, 3H).

### Example 43: 5-((1S,2R)-1-(6-chloro-1,1-dioxido-4-((S)-tetrahydro-2H-pyran-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using (S)-tetrahydro-2H-pyran-3-amine hydrochloride instead of 3-methoxypropylamine to obtain 5-((1S,2R)-1-(6-chloro-1,1-dioxido-4-((R)-tetrahydro-2H-pyran-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.018 g, yield 33%). ¹H NMR (400 MHz, Methanol-d₄) δ 7.57 (d, J=8.4 Hz, 1H), 6.99 (dd, J=8.4, 5.7 Hz, 1H), 6.93 (d, J=1.8 Hz, 1H), 6.81 (dd, J=8.4, 1.8 Hz, 1H), 6.72 (dd, J=12.1, 8.4 Hz, 1H), 5.37 (dd, J=11.9, 1.7 Hz, 1H), 5.30 (d, J=14.3 Hz, 1H), 5.22 (d, J=14.3 Hz, 1H), 4.00-3.91 (m, 1H), 3.91-3.85 (m, 2H), 3.81 (td, J=10.3, 5.1 Hz, 1H), 3.57-3.44 (m, 2H), 2.37 (s, 3H), 2.22 (s, 3H), 2.18-2.03 (m, 2H), 1.92-1.81 (m, 2H), 1.46 (dd, J=6.9, 1.1 Hz, 3H). LC-MS: m/z=549.17 [M−H]−.

### Example 44: 5-((1S,2R)-1-(6-chloro-1,1-dioxido-4-((R)-tetrahydro-2H-pyran-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using (R)-tetrahydro-2H-pyran-3-amine hydrochloride instead of 3-methoxypropylamine to obtain 5-((1S,2R)-1-(6-chloro-1,1-dioxido-4-((R)-tetrahydro-2H-pyran-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.0056 g, yield 23%). ¹H NMR (400 MHz, Methanol-d₄) δ 7.57 (d, J=8.4 Hz, 1H), 6.99 (dd, J=8.4, 5.7 Hz, 1H), 6.86 (d, J=1.9 Hz, 1H), 6.80 (dd, J=8.4, 1.8 Hz, 1H), 6.72 (dd, J=12.1, 8.4 Hz, 1H), 5.40 (dd, J=11.9, 1.8 Hz, 1H), 5.35 (d, J=14.5 Hz, 1H), 5.27 (d, J=14.5 Hz, 1H), 3.99-3.88 (m, 3H), 3.88-3.74 (m, 2H), 3.62 (ddd, J=11.7, 8.9, 3.5 Hz, 1H), 2.37 (s, 3H), 2.22 (s, 3H), 2.09-1.95 (m, 2H), 1.85-1.70 (m, 2H), 1.46 (dd, J=6.9, 1.2 Hz, 3H). LC-MS: m/z=549.17 [M−H]−.

### Example 45: 5-((1S,2R)-1-(6-chloro-4-(2-methoxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using 2-methoxyethylamine instead of 3-methoxypropylamine to 5-((1S,2R)-1-(6-chloro-4-(2-methoxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one. ¹H NMR (300 MHz, DMSO-d₆) δ 7.57 (d, J=8.2 Hz, 1H), 7.03-6.95 (m, 1H), 6.79 (d, J=9.5 Hz, 2H), 6.72 (dd, J=12.1, 8.4 Hz, 1H), 5.49-5.38 (m, 2H), 5.06 (d, J=14.8 Hz, 1H), 3.91 (dq, J=13.4, 6.8 Hz, 1H), 3.71-3.47 (m, 4H), 3.37 (s, 3H), 2.38 (s, 3H), 2.22 (s, 3H), 1.45 (d, J=6.9 Hz, 3H).

LC-MS: m/z=523.12 [M−H]−.

### Example 46: 5-((1S,2R)-1-(6-chloro-4-(cyclopropanecarbonyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

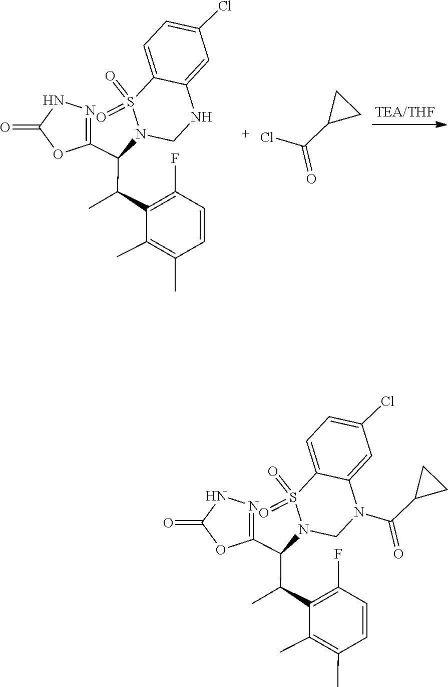 [SMILES: Cc1ccc(F)c([C@@H](C)[C@@H](c2n[nH]c(=O)o2)N2CNc3cc(Cl)ccc3S2(=O)=O)c1C ; O=C(Cl)C1CC1 ; Cc1ccc(F)c([C@@H](C)[C@@H](c2n[nH]c(=O)o2)N2CN(C(=O)C3CC3)c3cc(Cl)ccc3S2(=O)=O)c1C]

Cyclopropyl acid chloride (1.00 eq, 0.0045 mL, 0.043 mmol) is added to a solution of 5-((1S,2R)-1-(6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one(see example 8) (1.00 eq, 20 mg, 0.043 mmol) and triethylamine (3.00 eq, 0.018 mL, 0.129 mmol) in THF (0.5 mL). After 5 min, LC/MS shows no remaining starting material. The reaction mixture is poured in water and extracted 2 time with EtOAc. The combined organic phases are washed with brine, dried over MgSO₄, and concentrated under vacuum. The crude is purified on silica gel on Isco instrument using a gradient 0 to 50% EtOAc in Hexanes and yielding 18 mg of white solid (78%). ¹H NMR (300 MHz, Methanol-d₄) δ 7.60 (d, J=8.5 Hz, 1H), 7.56-7.54 (d, 1H) 7.06-7.03 (dd, J=8.4, 5.8 Hz, 1H), 6.86-6.82 (d, 1H), 6-75-6.34 (dd, 1H), 6.69 (d, 1H) 5.32-5.34 (dt, J=11.7, 1.2 Hz, 1H), 5.23-5.19 (dd, J=14.6 Hz, 1H), 5.11-5.08 (d, J=14.6 Hz, 1H), 3.98-3.95 (m, 1H), 2.35 (s, 3H), 2.25 (s, 3H), 2.13-2.10 (m, 1H), 1.36-1.37 (d, 3H), 1.00-0.92 (m, J=4H). LC-MS: m/z=[M−H]+=534.9.

### Example 47: 5-((1S,2R)-1-(6-chloro-4-(1-methylpiperidin-4-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using 4-amino-1-methylpiperidine instead of 3-methoxypropylamine to obtain 5-((1S,2R)-1-(6-chloro-4-(1-methylpiperidin-4-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.0026 g, yield 13%). ¹H NMR (300 MHz, Methanol-d₄) δ 7.56 (d, J=8.4 Hz, 1H), 7.03-6.95 (m, 1H), 6.91 (s, 1H), 6.79 (d, J=8.4 Hz, 1H), 6.72 (dd, J=12.1, 8.4 Hz, 1H), 5.40 (d, J=11.8 Hz, 1H), 5.18 (s, 2H), 3.99-3.87 (m, 1H), 3.84-3.71 (m, 1H), 3.15-3.00 (m, 2H), 2.40 (s, 3H), 2.37 (s, 3H), 2.34-2.25 (m, 1H), 2.22 (s, 3H), 2.16-2.01 (m, 2H), 1.98-1.84 (m, 3H), 1.47 (d, J=6.8 Hz, 3H).

LC-MS: m/z=[M−H]+=564.21 Example 48: 5-((1S,2R)-1-(6-chloro-4-(3-methoxypropyl)-1,1-dioxido-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

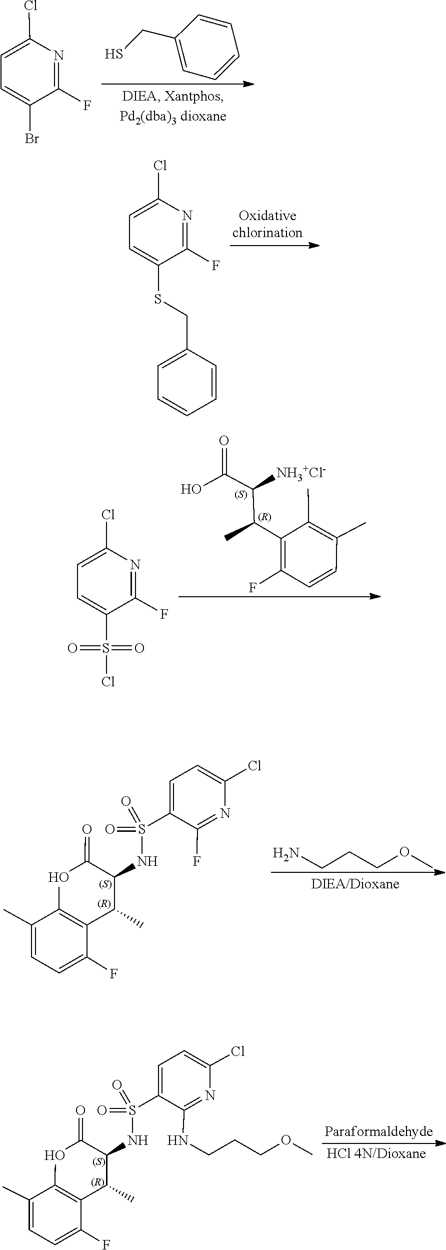

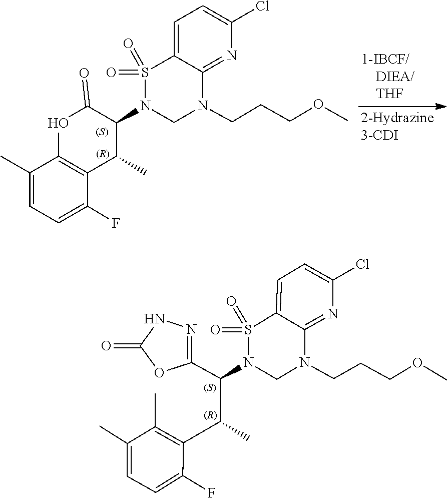

### Step 1: Synthesis of 3-(benzylthio)-6-chloro-2-fluoropyridine

To a degassed and argon purged solution of 3-Bromo-6-chloro-2-fluoropyridine (5.0 g, 23.7 mmol, 1.0 eq) in anhydrous dioxane (200 ml, 20 vol) were added N,N-Diisopropylethylamine (DIEA) (8.5 ml, 47.5 mmol, 2.0 eq), tris(dibenzylideneacetone)dipalladium(0) (Pd₂(dba)₃) (1.09 g, 1.19 mmol, 0.05 eq) and 4,5-bis(diphenylphosphino)-9,9-dimethylxanthene (Xantphos) (1.38 g, 2.38 mmol, 0.1 eq). The mixture was degassed again and purged with argon and the solution was stirred 100° C. in an oil bath. After 10 mins, benzyl mercaptan (5.9 g, 47.5 mmol, 2.0 eq) was added dropwise. The reaction was continued at 100° C. overnight. Upon completion, the solution was cooled to room temperature and passed through celite. The filtrate was concentrated and purified by FCC (0-5% of EtOH in Hexanes) to afford 3-(benzylthio)-6-chloro-2-fluoropyridine (13.0 g, 20.5 mmol, yield 86%).

### Step 2: Synthesis of 6-chloro-2-fluoropyridine-3-sulfonyl chloride

3-(benzylthio)-6-chloro-2-fluoropyridine in MeCN (80 ml, 40 vol) and water (2 ml, 1.0 vol) was added glacial acetic acid (0.225 ml, 3.9 mmol, 1.25 eq), followed by portion wise addition of 1,3-Dichloro-5,5-dimethylhydantoin (1.24 g, 6.3 mmol, 2.0 eq). The reaction was continued at room temperature and monitored by TLC and LC-MS for the next 1.5 h until starting material was completely consumed. Upon completion, the mixture was poured into water and extracted with MTBE. Combined organic layers were washed with brine and dried over Na₂SO₄. The solvent was removed in-vacuo to obtain a crude residue which was purified by FCC (5% EtOAc in Hexanes) to obtain 6-chloro-2-fluoropyridine-3-sulfonyl chloride (0.52 g, 2.26 mmol, yield 72%).

### Step 3: Synthesis of (2S,3R)-2-((6-chloro-2-fluoropyridine)-3-sulfonamido)-3-(6-fluoro-2,3-dimethylphenyl)butanoic acid

(2S,3R)-2-amino-3-(6-fluoro-2,3-dimethylphenyl)butanoic acid hydrochloride (0.6 g, 2.3 mmol, 1.0 eq) was suspended in a mixture of water and dioxane (v/v=(11 mL). To this, triethylamine (3.2 mL, 23 mmol, 3.0 eq) was added and the solution was cooled to 0° C., after which 6-chloro-2-fluoropyridine-3-sulfonyl chloride (0.52 g, 2.3 mmol, 1.0 eq) dissolved in a small volume of DCM was added portion wise. The reaction was continued at room temperature until the desired product formed completely after 3 hours. The solution was cooled in an ice bath and was slowly acidified with 1.0 M HCl. The mixture was extracted with EtOAc. Combined organic layers were washed with brine, dried over Na₂SO₄, filtered, and concentrated under reduced pressure to obtain (2S,3R)-2-(6-chloro-2-fluoropyridine-3-sulfonamido)-3-(6-fluoro-2,3-dimethylphenyl)butanoic acid (0.62 g, 1.4 mmol, yield 62%). The residue was used in subsequent reactions without further purification.

### Step 4: Synthesis of (2S,3R)-2-((6-chloro-2-((3-methoxypropyl)amino)pyridine)-3-sulfonamido)-3-(6-fluoro-2,3-dimethylphenyl)butanoic acid

(2S,3R)-2-(4-chloro-2-fluorobenzenesulfonamido)-3-(6-fluoro-2,3-dimethylphenyl)butanoic acid (112 mg, 0.269 mmol, 1.0 eq) was dissolved in anhydrous dioxane (2.69 mL). N,N-Diisopropylethylamine (DIPEA) (103 μL, 0.591 mmol, 2.2 eq) and 3-Methoxypropylamine (359 mg, 4.031 mmol, 15.0 eq) were added to the solution. Reaction mixture was stirred at 80° C. overnight. Upon completion the mixture was poured into 10% aq NaH₂PO₄ solution and extracted with EtOAc. Combined organic layers were washed with brine and dried over sodium sulfate. Solvent was removed under reduced pressure to give (2S,3R)-2-{4-chloro-2-[(3-methoxypropyl)amino]benzenesulfonamido}-3-(6-fluoro-2,3-dimethylphenyl)butanoic acid (0.146 g, 0.270 mmol, yield 100%), which was used in the next step without further purification.

### Step 5: Synthesis of (2S,3R)-2-(6-chloro-4-(3-methoxypropyl)-1,1-dioxido-3,4-dihydro-2H-pyrido[2,3-e][1,2,4]thiadiazin-2-yl)-3-(6-fluoro-2,3-dimethylphenyl)butanoic acid

(2S,3R)-2-{4-chloro-2-[(3-methoxypropyl)amino]benzenesulfonamido}-3-(6-fluoro-2,3-dimethylphenyl)butanoic acid (0.116 g, 0.214 mmol, 1.0 eq) was dissolved in anhydrous dioxane (5.8 mL), followed by the addition of paraformaldehyde (0.032 g, 1.069 mmol, 5.0 eq) and 4.0 M HCl in dioxane (214 μL, 0.428 mmol, 2.0 eq) and the resulting mixture was allowed to stir at 60° C. overnight. After the reaction was complete, the mixture was cooled down, diluted with water, and extracted using EtOAc. The organics were combined, washed with brine, and dried over MgSO₄. Solvents were removed in vacuo. The residue was purified by FCC (0-6% MeOH in DCM) to give (2S,3R)-2-(6-chloro-4-(3-methoxypropyl)-1,1-dioxido-3,4-dihydro-2H-pyrido[2,3-e][1,2,4]thiadiazin-2-yl)-3-(6-fluoro-2,3-dimethylphenyl)butanoic acid (0.12 g, 0.21 mmol, yield 99/a).

### Step 6: Synthesis of 5-((1S,2R)-1-(6-chloro-4-(3-methoxypropyl)-1,1-dioxido-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

(2S,3R)-2-(6-chloro-4-(3-methoxypropyl)-1,1-dioxido-3,4-dihydro-2H-pyrido[2,3-e][1,2,4]thiadiazin-2-yl)-3-(6-fluoro-2,3-dimethylphenyl)butanoic acid (69 mg, 0.12 mmol, 1.0 eq) was dissolved in anhydrous THF (2 mL) and cooled to −10° C. DIEA (24 μL, 0.135 mmol, 1.1 eq) was added, followed by dropwise addition of isobutyl-chloroformate (20 μL, 0.15 mmol, 1.2 eq). The reaction was continued at −10° C. for 30 min. Upon complete consumption of substrate, hydrazine monohydrate (31 μL, 0.61 mmol, 5.0 eq) was added dropwise and stirring was continued for 1 h. After formation of hydrazide was complete, CDI (159 mg, 0.98 mmol, 8.0 eq) was added portion wise and the reaction was continued at ambient temperature for 1 h. The mixture was poured into water and extracted with EtOAc. Organic layers were combined, washed with brine, dried over sodium sulfate and the solvent was removed under reduced pressure. The residue was purified by pTLC (2% MeOH in DCM) to afford the title compound (0.024 g, 0.044 mmol, yield 36%). ¹H NMR (400 MHz, Methanol-d₄) δ 7.89 (d, J=8.0 Hz, 1H), 7.00 (dd, J=8.4, 5.8 Hz, 1H), 6.76 (d, J=8.1 Hz, 1H), 6.72 (dd, J=12.4, 8.5 Hz, 1H), 5.43-5.37 (m, 2H), 5.16 (d, J=14.8 Hz, 1H), 4.06 (dt, J=13.9, 6.9 Hz, 1H), 3.92 (dq, J=13.3, 6.7 Hz, 1H), 3.42 (t, J=5.9 Hz, 2H), 3.15 (dt, J=14.2, 7.0 Hz, 1H), 2.39 (s, 3H), 2.23 (s, 3H), 1.93-1.83 (m, 2H), 1.45 (dd, J=7.0, 1.1 Hz, 3H). LC-MS: m/z=540.13 [M+H]+; 538.32 [M−H]−.

### Example 49: 5-((1S,2R)-1-(6-chloro-1,1-dioxido-4-((tetrahydrofuran-3-yl)methyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using 3-(aminomethyl)tetrahydrofuran instead of 3-methoxypropylamine to obtain 6-chloro-2-[(1S,2R)-2-(6-fluoro-2,3-dimethylphenyl)-1-(5-oxo-4,5-dihydro-1,3,4-oxadiazol-2-yl)propyl]-4-[(oxolan-3-yl)methyl]-3,4-dihydro-2H-1λ⁶,2,4-benzothiadiazine-1,1-dione (0.035 g, yield 17%). ¹H NMR (300 MHz, Methanol-d₄) δ 7.58 (d, J=8.6 Hz, 1H), 6.98 (t, J=7.2 Hz, 1H), 6.79 (d, J=5.1 Hz, 2H), 6.76-6.66 (m, 1H), 5.52-5.34 (m, 2H), 5.17 (dd, J=14.6, 9.4 Hz, 1H), 4.03-3.86 (m, 2H), 3.86-3.70 (m, 2H), 3.64-3.44 (m, 2H), 3.12 (dt, J=15.2, 7.5 Hz, 1H), 2.74-2.59 (m, 1H), 2.38 (s, 3H), 2.22 (s, 3H), 2.17-2.03 (m, 1H), 1.81-1.56 (m, 1H), 1.46 (d, J=6.9 Hz, 3H). LC-MS: m/z=549.17 [M−H]−.

### Example 50: 5-((1S,2R)-1-(6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

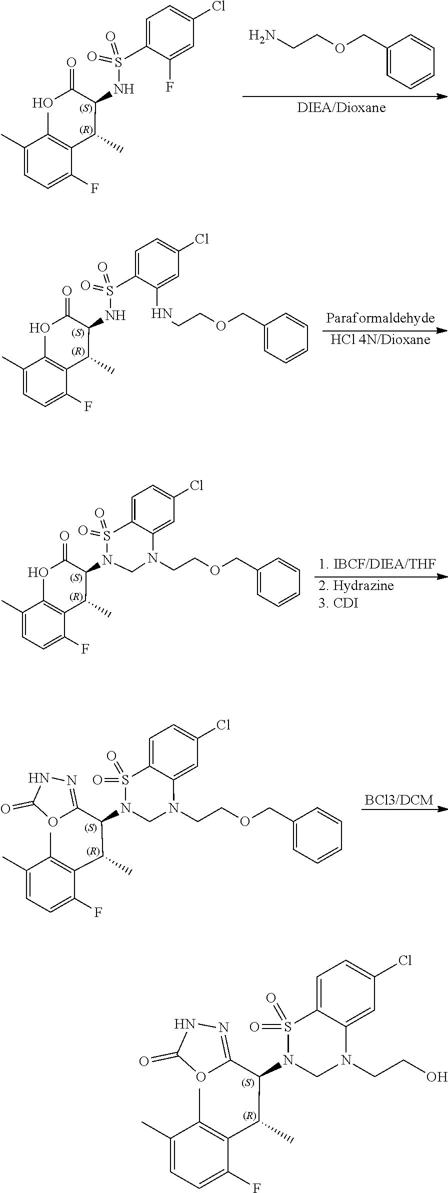

The title compound was synthesized following the procedure described for Ex 4 using 2-benzyloxyethylamine in substitution of 2-methoxypropylamine to obtain the oxadiazolone compound 5-((1S,2R)-1-(4-(2-(benzyloxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.157 g, 0.25 mmol, yield 84%). Next, 5-((1S,2R)-1-(4-(2-(benzyloxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.2 g, 0.316 mmol, 1.0 eq) was dissolved in anhydrous DCM (4.0 ml, 20 vol). To this, Boron trichloride, 1.0 M in methylene chloride (0.73 ml, 0.73 mmol, 2.3 eq) was added dropwise at −50 C under argon and stirred for 30 minutes until completion. The reaction mixture was cooled in an ice bath and quenched with sat. NaHCO₃. The solution was extracted with DCM and the organics were combined, washed with brine, and dried over MgSO₄. The crude obtained was further purified by FCC (50% EtOAc in Hexanes) and then by RPFCC to 5-((1S,2R)-1-(6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.055 g, yield 34%). ¹H NMR (400 MHz, Methanol-d₄) δ 7.60-7.56 (m, 1H), 7.00 (dd, J=8.4, 5.7 Hz, 1H), 6.79 (d, J=7.9 Hz, 2H), 6.72 (dd, J=12.1, 8.4 Hz, 1H), 5.55 (d, J=14.9 Hz, 1H), 5.44 (dd, J=11.8, 1.6 Hz, 1H), 5.09 (d, J=14.9 Hz, 1H), 3.91 (dq, J=13.4, 6.8 Hz, 1H), 3.79-3.65 (m, 2H), 3.61 (dt, J=15.5, 4.1 Hz, 1H), 3.29-3.22 (m, 1H), 2.39 (s, 3H), 2.23 (s, 3H), 1.45 (dd, J=6.9, 1.1 Hz, 3H). LC-MS: m/z=509.30 [M−H]−.

### Example 51: 5-((1S,2R)-1-(6-chloro-4-(1-methylpiperidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using 3-amino-1-methylpiperidine instead of 3-methoxypropylamine to obtain: 5-((1S,2R)-1-(6-chloro-4-(1-methylpiperidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.058 g, yield 24%). ¹H NMR (400 MHz, Methanol-d₄) δ 7.56 (d, J=8.4 Hz, 1H), 6.99 (dd, J=8.5, 5.7 Hz, 1H), 6.93 (d, J=1.8 Hz, 1H), 6.79 (dd, J=8.4, 1.8 Hz, 1H), 6.72 (dd, J=12.1, 8.4 Hz, 1H), 5.38 (dd, J=11.9, 1.7 Hz, 1H), 5.26 (d, J=14.2 Hz, 1H), 5.20 (d, J=14.2 Hz, 1H), 3.93 (dq, J=13.1, 6.7 Hz, 1H), 3.87-3.77 (m, 1H), 2.91 (dd, J=21.6, 11.1 Hz, 2H), 2.42 (s, 3H), 2.38 (s, 3H), 2.37-2.33 (m, 1H), 2.22 (s, 3H), 2.13 (dd, J=13.7, 11.0 Hz, 1H), 1.98-1.92 (m, 1H), 1.92-1.85 (m, 1H), 1.85-1.76 (m, 1H), 1.75-1.65 (m, 1H), 1.46 (dd, J=6.9, 1.1 Hz, 3H). LC-MS: m/z=564.18 [M+H]+.

### Example 52: 5-((1S,2R)-1-(6-chloro-1,1-dioxido-4-(((R)-tetrahydrofuran-2-yl)methyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-4 using (R)-(−)-tetrahydrofurfurylamine instead of 2-methoxypropylamine to obtain 5-((1S,2R)-1-(6-chloro-1,1-dioxido-4-(((R)-tetrahydrofuran-2-yl)methyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.0078 g, yield 36%). ¹H NMR (400 MHz, Methanol-d₄) δ 7.58 (dd, J=8.4, 2.6 Hz, 1H), 6.98 (dd, J=8.5, 5.7 Hz, 1H), 6.81 (d, J=1.7 Hz, 1H), 6.78 (dd, J=8.4, 1.8 Hz, 1H), 6.71 (dd, J=12.0, 8.3 Hz, 1H), 5.58 (d, J=15.0 Hz, 1H), 5.46 (d, J=12.0 Hz, 1H), 5.03 (d, J=15.0 Hz, 1H), 4.14-4.05 (m, 1H), 3.98-3.86 (m, 2H), 3.75 (td, J=7.9, 6.0 Hz, 1H), 3.66 (dd, J=15.4, 2.2 Hz, 1H), 3.03 (dd, J=15.6, 9.7 Hz, 1H), 2.39 (s, 3H), 2.22 (s, 3H), 2.13-2.03 (m, 1H), 2.02-1.87 (m, 2H), 1.60-1.50 (m, 1H), 1.45 (dd, J=6.9, 1.1 Hz, 3H).

LC-MS: m/z=551.19 [M+H]+.

### Example 53 and 54: 5-((1S,2R)-1-(7-chloro-6-((S)-1-(dimethylamino)ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one and 5-((1S,2R)-1-(7-chloro-6-((R)-1-(dimethylamino)ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

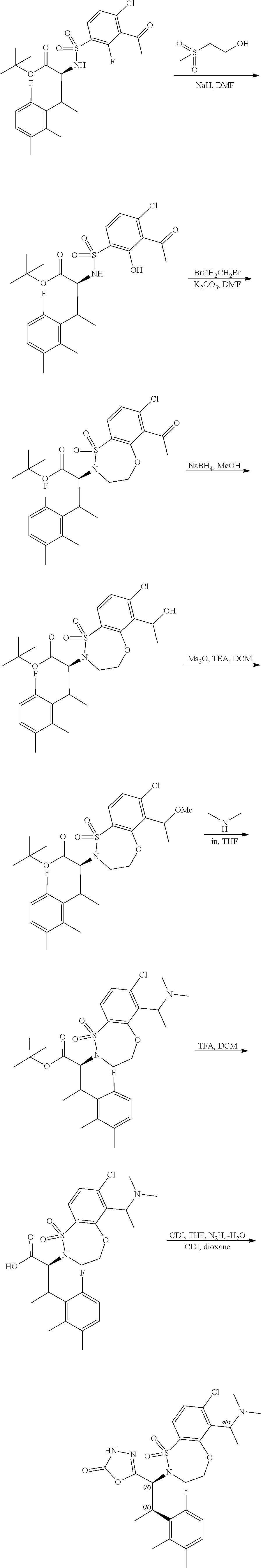

### Step 1: Synthesis of tert-butyl (2S)-2-((3-acetyl-4-chloro-2-hydroxyphenyl) sulfonamido)-3-(6-fluoro-2,3-dimethylphenyl) butanoate

To a solution of tert-butyl (S)-2-(3-acetyl-4-chloro-2-fluorobenzenesulfonamido)-3-(6-fluoro-2,3-dimethylphenyl) butanoate (see ex 25) (1.6 g, 3.101 mmol, 1 equiv) in DMF was added NaH (496 mg, 12.4 mmol, 4 equiv, 60%) at 0° C. The mixture was stirred for 15 min. 2-methanesulfonylethanol (770 mg, 6.2 mmol, 2 equiv) was added and the mixture was allowed to warm to RT and stirred 2 h at room temperature under nitrogen atmosphere. The reaction was quenched with sat. NH₄Cl (aq., 30 ml) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 0% to 100% gradient in 20 min; detector, UV 220 nm to afford tert-butyl (2S)-2-(3-acetyl-4-chloro-2-hydroxybenzenesulfonamido)-3-(6-fluoro-2,3-dimethylphenyl)butanoate (490 mg, 30.7%).

### Step 2: Synthesis of tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate

Into a 40 mL sealed tube were added tert-butyl (2S)-2-(3-acetyl-4-chloro-2-hydroxybenzenesulfonamido)-3-(6-fluoro-2,3-dimethylphenyl) butanoate (490 mg, 0.95 mmol, 1 equiv), DMF (8 mL), dibromoethane (123 μL, 1.43 mmol, 1.5 equiv) and K₂CO₃ (395 mg, 2.9 mmol, 3 equiv) at room temperature. The resulting mixture was stirred overnight at 65° C. The reaction was quenched with sat. NH₄C1 (aq., 30 ml) at room temperature. The resulting mixture was extracted with EtOAc (3×10 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (10:1) to afford tert-butyl (2S)-2-(6-acetyl-7-chloro-1,1-dioxido-3,4-dihydro-2H-benzo[b][1,4,5]oxathiazepin-2-yl)-3-(6-fluoro-2,3-dimethylphenyl)butanoate (420 mg, 81.6%).

### Step 3: Synthesis of tert-butyl (2S)-2-(7-chloro-6-(1-hydroxyethyl)-1,1-dioxido-3,4-dihydro-2H-benzo[b][1,4,5]oxathiazepin-2-yl)-3-(6-fluoro-2,3-dimethylphenyl)butanoate

To a stirred solution of afford tert-butyl (2S)-2-(6-acetyl-7-chloro-1,1-dioxido-3,4-dihydro-2H-benzo[b][1,4,5] oxathiazepin-2-yl)-3-(6-fluoro-2,3-dimethylphenyl)butanoate (400 mg, 0.741 mmol, 1 equiv) in MeOH was added NaBH₄ (140 mg, 3.7 mmol, 5 equiv) in portions at 0° C. The resulting mixture was stirred for 30 min at room temperature. The reaction was quenched with Water (50 ml) at room temperature. The resulting mixture was extracted with EtOAc (2×50 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EtOAc (9:1) to afford tert-butyl (2S)-2-(7-chloro-6-(1-hydroxyethyl)-1,1-dioxido-3,4-dihydro-2H-benzo[b][1,4,5] oxathiazepin-2-yl)-3-(6-fluoro-2,3-dimethylphenyl)butanoate (270 mg, 67.3%).

### Step 4: Synthesis of tert-butyl (2S)-2-(7-chloro-6-(1-((methylsulfonyl)oxy)ethyl)-1,1-dioxido-3,4-dihydro-2H-benzo[b][1,4,5]oxathiazepin-2-yl)-3-(6-fluoro-2,3-dimethylphenyl)butanoate

To a stirred solution of tert-butyl (2S)-2-[7-chloro-6-(1-hydroxyethyl)-1,1-dioxo-3,4-dihydro-5,1lambda6,2-benzoxathiazepin-2-yl]-3-(6-fluoro-2,3-dimethylphenyl)butanoate (260 mg, 0.480 mmol, 1 equiv) and DCM (4 mL) was added methanesulfonic anhydride (334.20 mg, 1.920 mmol, 4 equiv) dropwise at 0° C. The resulting mixture was stirred for 1 h at 0° C. under nitrogen atmosphere. The resulting mixture was washed with 3×50 mL of brine. The resulting mixture was concentrated under reduced pressure. This resulted in tert-butyl(2S)-2-{7-chloro-6-[1-(methanesulfonyloxy)ethyl]-1,1-dioxo-3,4-dihydro-5,1lambda6,2-benzoxathiazepin-2-yl}-3-(6-fluoro-2,3-dimethylphenyl)butanoate (340 mg, crude). The crude product was used in the next step directly without further purification.

### Step 5: Synthesis of tert-butyl (2S)-2-(7-chloro-6-(1-(dimethylamino) ethyl)-1,1-dioxido-3,4-dihydro-2H-benzo[b][1,4,5]oxathiazepin-2-yl)-3-(6-fluoro-2,3-dimethylphenyl)butanoate

To a stirred solution of tert-butyl(2S)-2-{7-chloro-6-[1-(methanesulfonyloxy)ethyl]-1,1-dioxo-3,4-dihydro-5,1lambda6,2-benzoxathiazepin-2-yl}-3-(6-fluoro-2,3-dimethylphenyl)butanoate (320 mg, 0.516 mmol, 1 equiv) in dimethylamine (2 M in THF) (4 mL, 2.580 mmol, 5 equiv) was added TEA (358.63 μL, 2.580 mmol, 5 equiv) at room temperature. The resulting mixture was stirred overnight at 65° C. The resulting mixture was concentrated under vacuum. This resulted in tert-butyl (2S)-2-(7-chloro-6-(1-(dimethylamino) ethyl)-1,1-dioxido-3,4-dihydro-2H-benzo[b][1,4,5]oxathiazepin-2-yl)-3-(6-fluoro-2,3-dimethylphenyl)butanoate (290 mg, crude). The crude product was used in the next step directly without further purification.

### Step 6: Synthesis of (2S)-2-(7-chloro-6-(1-(dimethylamino) ethyl)-1,1-dioxido-3,4-dihydro-2H-benzo[b][1,4,5]oxathiazepin-2-yl)-3-(6-fluoro-2,3-dimethylphenyl)butanoic acid

To a stirred solution of tert-butyl (2S)-2-(7-chloro-6-(1-(dimethylamino) ethyl)-1,1-dioxido-3,4-dihydro-2H-benzo[b][1,4,5]oxathiazepin-2-yl)-3-(6-fluoro-2,3-dimethylphenyl)butanoate (280 mg, 0.49 mmol, 1 equiv) in DCM was added TFA (500 μL, 49.1 mmol, 100 equiv) at room temperature. The resulting mixture was stirred for 1 h at room temperature. The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 220 nm. This resulted in (2S,3R)-2-{7-chloro-6-[1-(dimethylamino) ethyl]-1,1-dioxo-3,4-dihydro-5,1lambda6,2-benzoxathiazepin-2-yl}-3-(6-fluoro-2,3-dimethylphenyl) butanoic acid (165 mg, 65.4%) Step 7: Synthesis of 5-((1S,2R)-1-(7-chloro-6-((S)-1-(dimethylamino) ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one and 5-((1S,2R)-1-(7-chloro-6-((R)-1-(dimethylamino) ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

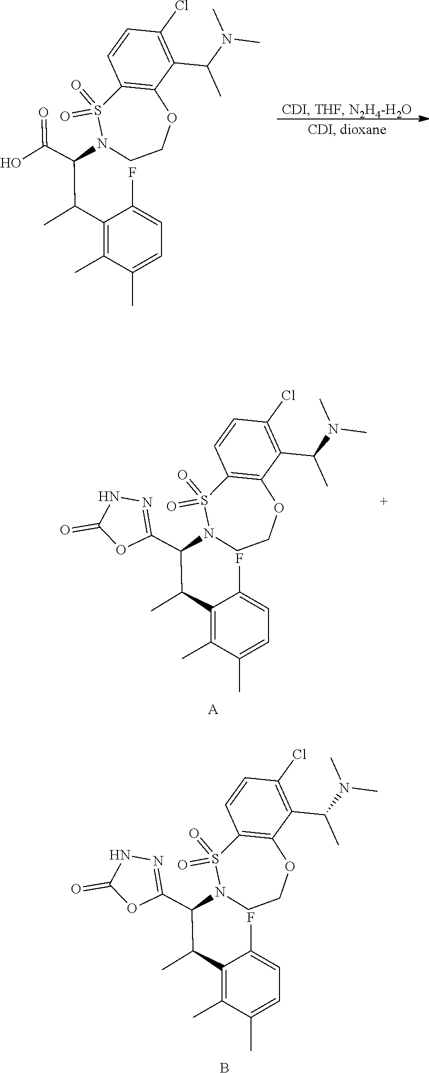

To a stirred solution of (2S)-2-{7-chloro-6-[1-(dimethylamino)ethyl]-1,1-dioxo-3,4-dihydro-5,1lambda6,2-benzoxathiazepin-2-yl}-3-(6-fluoro-2,3-dimethylphenyl)butanoic acid (116 mg, 0.23 mmol, 1 equiv) in THF (1 ml) is added CDI (367 mg, 2.26 mmol, 10 equiv) at room temperature. The resulting mixture was stirred for 1 h at room temperature. To the above mixture was added NH₂NH₂·H₂O (220 μL, 4.52 mmol, 20 equiv) dropwise at 0° C. The resulting mixture was stirred for additional 1 h at ° C. The reaction was quenched with water (20 ml) at room temperature. The resulting mixture was extracted with EtOAc (3×20 mL). The combined organic layers were washed with brine (2×30 mL), dried over anhydrous Na₂SO₄. After filtration, the filtrate was concentrated under reduced pressure to afford a yellow solid. To a stirred solution of the yellow solid in 1,4-dioxane (2 ml) were added CDI (147 mg, 0.90 mmol, 4 equiv) in portions at room temperature. The resulting mixture was stirred for 1 h at room temperature. The reaction was quenched with water (3 ml) at room temperature. The residue was purified by reversed-phase flash chromatography with the following conditions: Column: XBridge Shield RP18 OBD Column, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 26% B to 56% B in 9 min, 56% B; Wave Length: 254 nm; RT1 (min): 7.2 different diastereoisomers were isolated. The stereochemistry of the 2 first stereocenters was deducted from the ¹H NMR while the third stereocenter was arbitrary assigned:

stereoisomer A:5-((1S,2R)-1-(7-chloro-6-((S)-1-(dimethylamino) ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (22.1 mg, 16.9%). (ES, m/z): [M+H]+=553.15. ¹H NMR (300 MHz, Methanol-d₄) δ 7.85-7.75 (d, J=8.6 Hz, 1H), 7.39-7.36 (d, J=8.5 Hz, 1H), 6.98-6.95 (dd, J=8.5, 5.7 Hz, 1H), 6.75-6.68 (dd, J=12.2, 8.4 Hz, 1H), 5.57-5.53 (d, J=11.7 Hz, 1H), 4.70-4.53 (s, 5H), 4.41-4.31 (d, J=6.9 Hz, 1H), 4.16-4.08 (d, J=9.9 Hz, 1H), 3.97-3.85 (m, 2H), 3.65-3.53 (s, 1H), 2.37-2.33 (s, 6H), 2.33 (s, 3H), 2.30-2.15 (s, 3H), 1.56-1.45 (m, 6H).

stereoisomer B:5-((1S,2R)-1-(7-chloro-6-((R)-1-(dimethylamino) ethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (17.3 mg, 12.71%). LC-MS: (ES, m/z): [M+H]+=553.15. ¹H NMR (300 MHz, Methanol-d₄) δ 7.75-7.65 (d, J=8.5 Hz, 1H), 7.40-7.25 (d, J=8.6 Hz, 1H), 7.00-6.90 (dd, J=8.4, 5.7 Hz, 1H), 6.80-6.65 (dd, J=12.1, 8.4 Hz, 1H), 5.60-5.45 (d, J=11.6 Hz, 1H), 4.58-4.55 (s, 1H), 4.54-4.40 (m, 1H), 4.23-3.99 (m, 2H), 3.95-3.80 (dd, J=12.3, 6.4 Hz, 2H), 3.79-3.60 (s, 1H), 2.40-2.30 (s, 3H), 2.30-2.15 (d, J=14.1 Hz, 9H), 1.56-1.37 (m, 6H).

### Example 55: 5-((1S,2R)-1-(7-chloro-6-((dimethylamino)methyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

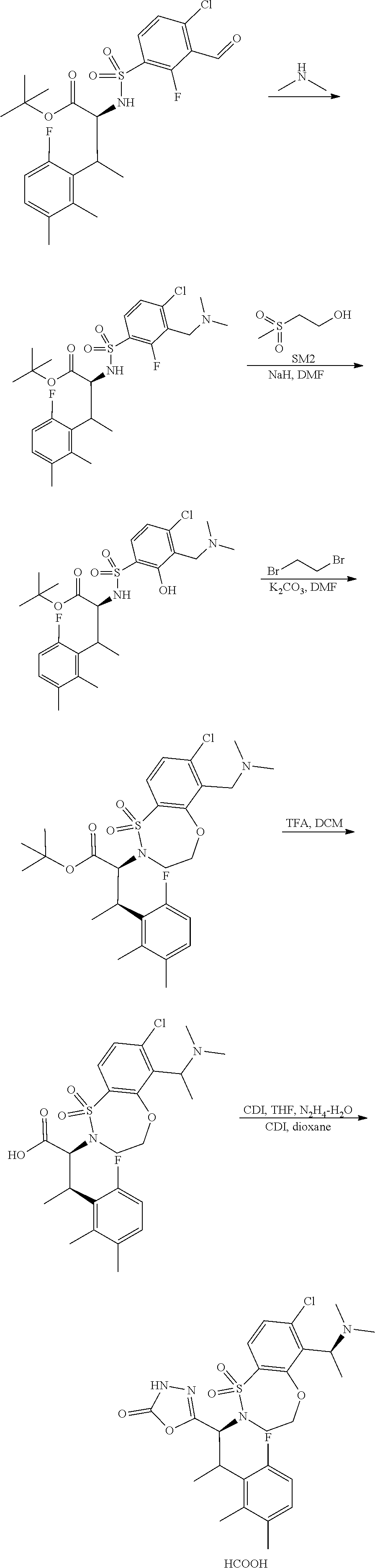

### Step 1: Synthesis of tert-butyl (2S)-2-((4-chloro-3-((dimethylamino)methyl)-2-fluorophenyl)sulfonamido)-3-(6-fluoro-2,3-dimethylphenyl)butanoate

To a stirred solution of tert-butyl (2S)-2-((4-chloro-2-fluoro-3-formylphenyl) sulfonamido)-3-(6-fluoro-2,3-dimethylphenyl) butanoate (0.421 g, 0.84 mmol, 1 equiv) in DCM (20 mL) was added dimethylamine (2 M in THF) (2.1 ml, 4.2 mmol, 5 equiv) followed by sodium triacetoxyborohydride (0.53 g, 2.5 mmol, 3 equiv) at room temperature. The resulting mixture was stirred overnight at room temperature. The reaction was quenched with water (50 ml) at room temperature. The resulting mixture was extracted with CH₂Cl₂ (3×100 mL). The combined organic layers were washed with brine (1×300 mL), dried over anhydrous MgSO₄. After filtration, the filtrate was concentrated under reduced pressure. The residue was purified by silica gel column chromatography, eluted with PEI/EtOAc (9:1) to afford [(3-bromo-6-chloro-2-fluorophenyl) methyl]dimethylamine (327 mg, 73.5%).

### Step 2: Synthesis of tert-butyl (2S)-2-((4-chloro-3-((dimethylamino)methyl)-2-hydroxyphenyl) sulfonamido)-3 (6-fluoro-2,3-dimethylphenyl) butanoate

To a stirred solution of tert-butyl (2S)-2-{4-chloro-3-[(dimethylamino)methyl]-2-fluorobenzenesulfonamido}-3-(6-fluoro-2,3-dimethylphenyl) butanoate (460 mg, 0.87 mmol, 1 equiv) and 2-methanesulfonylethanol (323 mg, 2.6 mmol, 3 equiv) in DMF was added NaH (173 mg, 4.33 mmol, 5 equiv, 60%) in portions at 0° C. under nitrogen atmosphere. The resulting mixture was stirred overnight at 50° C. under nitrogen atmosphere. The reaction was quenched with water (10 ml) at room temperature. The residue was purified by reversed-phase flash chromatography with the following conditions: column, C18 silica gel; mobile phase, MeCN in Water (10 mmol/L NH₄HCO₃), 10% to 100% gradient in 10 min; detector, UV 254 nm. This resulted in tert-butyl (2S)-2-{4-chloro-3-[(dimethylamino)methyl]-2-hydroxybenzenesulfonamido}-3-(6-fluoro-2,3-dimethylphenyl)butanoate (190 mg, 41.5%).

### Step 3: Synthesis of tert-butyl (2S)-2-(7-chloro-6-((dimethylamino)methyl)-1,1-dioxido-3,4-dihydro-2H-benzo[b][1,4,5]oxathiazepin-2-yl)-3-(6-fluoro-2,3-dimethylphenyl)butanoate

To a stirred solution of tert-butyl (2S)-2-{4-chloro-3-[(dimethylamino)methyl]-2-hydroxybenzenesulfonamido}-3-(6-fluoro-2,3-dimethylphenyl) butanoate (270 mg, 0.51 mmol, 1 equiv) and K₂CO₃ (216 mg, 1.53 mmol, 3 equiv) in DMF (1 ml) was added dibromoethane (66 μL, 0.77 mmol, 1.5 equiv) dropwise at room temperature. The resulting mixture was stirred overnight at 60° C. The residue was purified by reversed-phase flash chromatography with the following conditions: column, C18 silica gel; mobile phase, MeCN in Water (10 mmol/L NH₄HCO₃), 10% to 100% gradient in 10 min; detector, UV 220 nm. This resulted in tert-butyl (2S)-2-(7-chloro-6-((dimethylamino)methyl)-1,1-dioxido-3,4-dihydro-2H-benzo[b][1,4,5]oxathiazepin-2-yl)-3-(6-fluoro-2,3-dimethylphenyl) butanoate (88 mg, 31.06%).

### Step 4: Synthesis of (2S)-2-(7-chloro-6-((dimethylamino)methyl)-1,1-dioxido-3,4-dihydro-2H-benzo[b][1,4,5]oxathiazepin-2-yl)-3-(6-fluoro-2,3-dimethylphenyl)butanoic acid

To a stirred solution of in tert-butyl (2S)-2-(7-chloro-6-((dimethylamino)methyl)-1,1-dioxido-3,4-dihydro-2H-benzo[b][1,4,5]oxathiazepin-2-yl)-3-(6-fluoro-2,3-dimethylphenyl) butanoate (88 mg, 0.025 mmol, 1 equiv) in DCM was added TFA (0.5 mL) at room temperature. The resulting mixture was stirred for 1 h at room temperature. The resulting mixture was concentrated under vacuum. The crude product was used in the next step directly without further purification.

### Step 5: Synthesis of 5-((1S,2R)-1-(7-chloro-6-((dimethylamino)methyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

To a stirred solution of (2S)-2-(7-chloro-6-((dimethylamino)methyl)-1,1-dioxido-3,4-dihydro-2H-benzo[b][1,4,5]oxathiazepin-2-yl)-3-(6-fluoro-2,3-dimethylphenyl)butanoic acid (100 mg, 0.20 mmol, 1 equiv) in THF was added CDI (162.5 mg, 1.000 mmol, 5 equiv) in portions at room temperature. The resulting mixture was stirred for 30 min at room temperature. To the above mixture was added hydrazine hydrate (97 μL, 2 mmol, 10 equiv) dropwise at 0° C. The resulting mixture was stirred for additional 20 min at 0° C. The reaction was quenched with Water/Ice (3 ml) at 0° C. The resulting mixture was extracted with EtOAc (2×2 mL). The combined organic layers were washed with brine (1×3 mL), dried over anhydrous Na₂SO₄. After filtration, the filtrate was concentrated under reduced pressure. The resulting mixture was diluted with 1,4-dioxane (0.5 mL). To the above mixture was added CDI (162.48 mg, 1.000 mmol, 5 equiv) at room temperature. The resulting mixture was stirred for additional 1 h at room temperature. The reaction was quenched with water (1 ml) at room temperature. The resulting mixture was concentrated under vacuum. This resulted in 5-((1S)-1-(7-chloro-6-((dimethylamino)methyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (110 mg, 101%). The crude product (60 mg was purified by Chiral-Prep-HPLC with the following conditions:Column, XSelect CSH Fluoro Phenyl, 30×150 mm, 5 μm; mobile phase, Water (0.1% FA) and ACN (16% ACN up to 46% in 8 min); Detector, uv 254&220 nm. This resulted in 5-((1S,2R)-1-(7-chloro-6-((dimethylamino)methyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one; formic acid (28.5 mg, 43.5%). LCMS: ES, m/z): M+H=539.1. 1H-NMR (300 MHz, Methanol-d₄) δ 8.44 (s, 1H), δ 7.83-7.80 (d, J=8.6 Hz, 1H), 7.42-7.39 (d, J=8.6 Hz, 1H), 7.12-6.97 (dd, J=8.4, 5.7 Hz, 1H), 6.76-6.69 (dd, J=12.1, 8.4 Hz, 1H), 5.56-5.51 (dd, J=11.6, 2.2 Hz, 1H), 4.68-4.63 (m, 1H), 4.02-3.86 (m, 6H), 2.52 (s, 6H), 2.33 (s, 3H), 2.21 (s, 3H), 1.45-1.43 (dd, J=6.9, 1.1 Hz, 3H).

### Example 56: 5-((1S,2R)-1-(4-acetyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-46 using Acetyl chloride instead of cyclopropyl acyl chloride to obtain 5-((1S,2R)-1-(4-acetyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one(0.037 g, yield 85%). ¹H NMR (300 MHz, DMSO-d6) δ 7.60 (s broad, 1H),7.57-7.55 (d, 1H) 7.06-7.03 (dd, J=8.4, 5.8 Hz, 1H), 6.86-6.82 (d, 1H), 6-75-6.74 (dd, 1H), 6.70 (s, 1H) 5.30-5.32 (d, J=11.7, 1H), 5.19-5.15 (dd, J=14.6 Hz, 1H), 5.09-5.06 (d, J=14.6 Hz, 1H), 3.99-3.93 (m, 1H), 2.35 (s, 3H), 2.18 (s, 3H), 2.12 (s, 3H), 1.36-1.34 (d, 3H). LC-MS: m/z=[M−H]+=508.93.

### Example 57: 5-((1S,2R)-1-(4-(azetidin-3-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

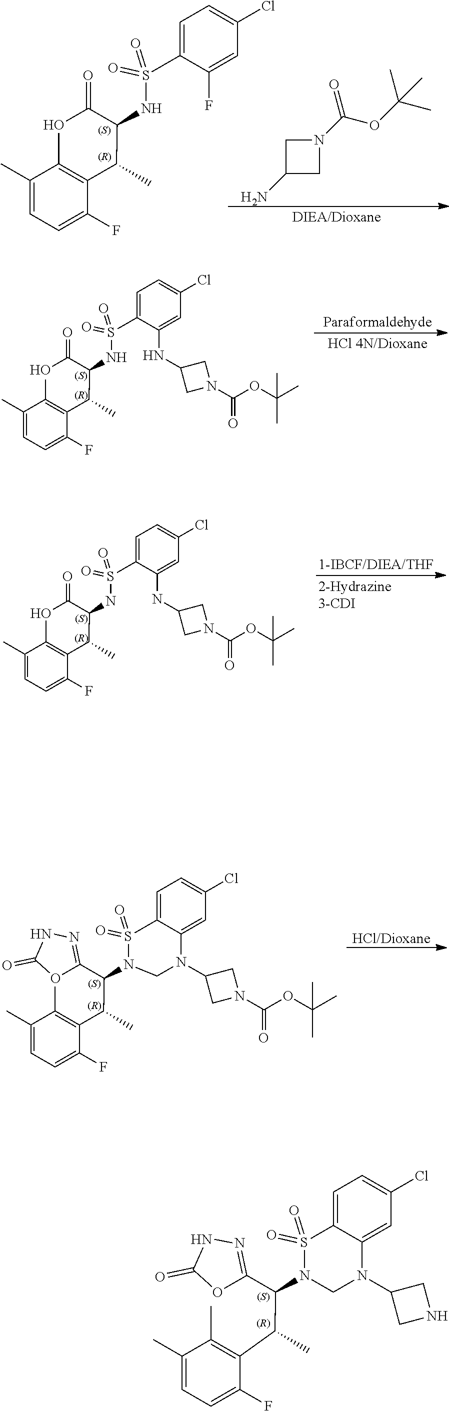

The Boc-protected variant of the title compound, tert-butyl 3-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)azetidine-1-carboxylate was synthesized following the procedure described for Ex-55 step 2 using 1-Boc-3-aminoazetidine instead of 3-methoxypropylamine. Next, tert-butyl 3-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)azetidine-1-carboxylate (0.052 g, 0.083 mmol, 1.0 eq) was dissolved in anhydrous dioxane (0.3 mL). To this, 4.0 M HCl in 1,4-dioxane (0.291 ml, 1.17 mmol, 14.0 eq) was added and the mixture was stirred at room temperature overnight. Upon completion of the reaction, the solvent was evaporated under reduced pressure and the residue was purified by RPFCC (50% MeCN in Water) and further purified by prep. HPLC to obtain the title compound 5-((1S,2R)-1-(4-(azetidin-3-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.017 g, yield 35%). ¹H NMR (400 MHz, Methanol-d₄) δ 8.51 (s, 21), 7.64 (d, J=8.4 Hz, 1H), 7.00 (dd, J=8.4, 5.8 Hz, 1H), 6.92 (dd, J=8.6, 1.7 Hz, 1H), 6.72 (dd, J=12.2, 8.4 Hz, 1H), 6.65 (s, 1H), 5.43 (d, J=13.2 Hz, 2H), 5.32 (d, J=14.2 Hz, 1H), 4.75-4.67 (m, 1H), 4.46 (t, J=9.4 Hz, 1H), 4.22-4.07 (m, 3H), 4.01-3.88 (m, 1H), 2.38 (s, 3H), 2.22 (s, 3H), 1.49 (d, J=6.8 Hz, 3H). LC-MS: m/z=[M−H]+=522.18

### Example 58: 5-((1S,2R)-1-(6-chloro-1,1-dioxido-4-((R)-pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

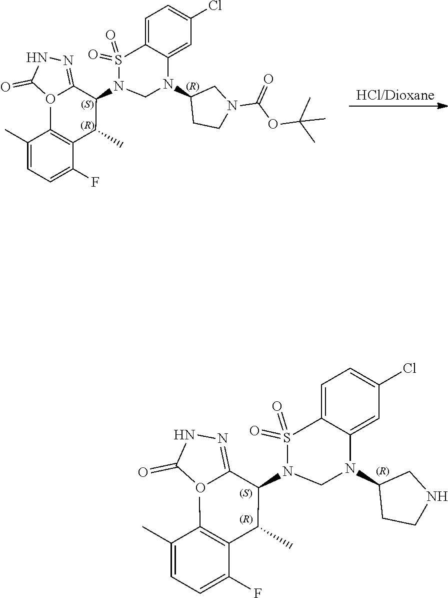

The Boc-protected variant tert-butyl (R)-3-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)pyrrolidine-1-carboxylate was synthesized following the procedure described in example 30 using (R)-(−)-1-Boc-3-aminopyrrolidine in place of 3-methoxypropylamine to obtain variant tert-butyl (R)-3-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)pyrrolidine-1-carboxylate (0.065 g, yield 69%). After deprotection the title compound 5-((1S,2R)-1-(6-chloro-1,1-dioxido-4-((R)-pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one was isolated (0.011 g, yield 17%). ¹H NMR (300 MHz, Methanol-d₄) δ 8.49 (s, 1H), 7.61 (d, J=8.4 Hz, 1H), 7.04-6.97 (m, 2H), 6.89 (d, J=8.4 Hz, 1H), 6.73 (dd, J=12.1, 8.4 Hz, 1H), 5.41 (d, J=11.8 Hz, 1H), 5.31 (d, J=14.0 Hz, 1H), 5.18 (d, J=14.1 Hz, 1H), 4.72-4.54 (m, 1H), 3.93 (td, J=13.5, 6.8 Hz, 1H), 3.63-3.36 (m, 3H), 3.28-3.23 (m, 1H), 2.37 (s, 3H), 2.34-2.23 (m, 2H), 2.22 (s, 3H), 1.49 (d, J=6.9 Hz, 3H). LC-MS: m/z=[M−H]+=536.18.

### Example 59: 5-((1S,2R)-1-(6-chloro-1,1-dioxido-4-((S)-pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the of example 58 using (S)-(−)-1-Boc-3-aminopyrrolidine to obtain 5-((1S,2R)-1-(6-chloro-1,1-dioxido-4-((S)-pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.007 g, yield 10%). ¹H NMR (400 MHz, Methanol-d₄) δ 8.53 (s, 1H), 7.62 (dd, J=8.5, 2.4 Hz, 1H), 7.02-6.96 (m, 2H), 6.88 (dd, J=8.4, 1.7 Hz, 1H), 6.72 (dd, J=12.1, 8.5 Hz, 1H), 5.41 (d, J=11.7 Hz, 1H), 5.30 (d, J=14.1 Hz, 1H), 5.26 (d, J=14.1 Hz, 1H), 4.52 (dt, J=14.6, 7.2 Hz, 1H), 4.02-3.88 (m, 1H), 3.54-3.40 (m, 2H), 3.30-3.23 (m, 1H), 3.15-3.05 (m, 1H), 2.47-2.37 (m, 2H), 2.37 (s, 3H), 2.22 (s, 3H), 1.48 (d, J=6.7 Hz, 3H). LC-MS: m/z=[M−H]+=536.45.

### Example 60: 5-((1S,2R)-1-(9-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

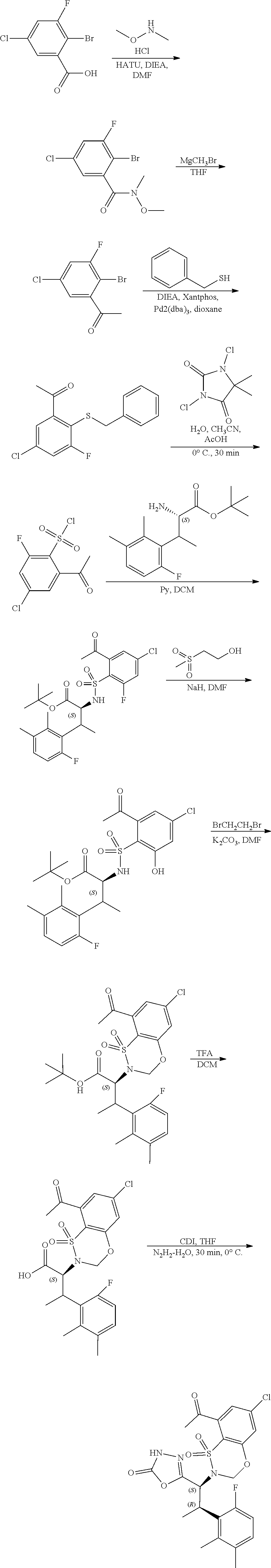

### Step 1: Synthesis of 2-bromo-5-chloro-3-fluoro-N-methoxy-N-methylbenzamide

Into a 250 mL round-bottom flask were added 2-bromo-5-chloro-3-fluorobenzoic acid (10 g, 39.46 mmol, 1 equiv), DMF (100 mL), N,O-dimethylhydroxylamine hydrochloride (5.77 g, 59.18 mmol, 1.5 equiv), HATU (22.5 g, 59.2 mmol, 1.5 equiv) and DIEA (20 mL, 118 mmol, 3 equiv) at room temperature. The resulting mixture was stirred for 5 h at room temperature. The reaction was quenched with sat. NH₄Cl (aq.) at room temperature. The resulting mixture was extracted with EtOAc (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EtOAc (7:1) to afford 1-(2-bromo-5-chloro-3-fluorophenyl) ethanone (9.1 g, 91.71%) as a yellow oil.

### Step 2: Synthesis of 1-(2-bromo-5-chloro-3-fluorophenyl) ethan-1-one

To a stirred solution of 2-bromo-5-chloro-3-fluoro-N-methoxy-N-methylbenzamide (9.5 g, 32 mmol, 1 equiv) in THF was added CH₃MgBr 3M (in Et₂O) (16 mL, 48 mmol, 1.5 equiv) dropwise at 0° C. under nitrogen atmosphere. The resulting mixture was stirred for 1 h at room temperature under nitrogen atmosphere. The reaction was quenched with sat. NH₄Cl (aq.) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EtOAc (1:1) to afford 1-(2-bromo-5-chloro-3-fluorophenyl) ethan-1-one (3.3 g, 41%).

### Step 3: Synthesis of 1-(2-(benzylthio)-5-chloro-3-fluorophenyl) ethan-1-one

Into a 8 mL vial were added 1-(2-bromo-5-chloro-3-fluorophenyl) ethanone (3.3 g, 13 mmol, 1 equiv), dioxane (35 mL), benzyl mercaptan (1.96 mL, 16.7 mmol, 1.3 equiv), DIEA (7 mL, 39.4 mmol, 3 equiv), Pd₂(dba)₃ (1.20 g, 1.3 mmol, 0.1 equiv) and Xantphos (1.52 g, 2.6 mmol, 0.2 equiv) at room temperature. The resulting mixture was stirred overnight at 110° C. under nitrogen atmosphere. The resulting mixture was filtered, the filter cake was washed with CH₂Cl₂ (3×30 mL). The filtrate was concentrated under reduced pressure. The residue was purified by silica gel column chromatography, eluted with PE/EtOAc (5:1) to afford 1-(2-(benzylthio)-5-chloro-3-fluorophenyl) ethan-1-one (2.1 g, 54.3%).

### Step 4: Synthesis of 2-acetyl-4-chloro-6-fluorobenzenesulfonyl chloride

To a stirred solution of 1-[2-(benzylsulfanyl)-5-chloro-3-fluorophenyl]ethanone (2.1 g, 7.1 mmol, 1 equiv) and H₂O (0.8 mL), AcOH (4 mL) in MeCN (20 mL) was added 1,3-dichloro-5,5-dimethylimidazolidine-2,4-dione (2.81 g, 14.2 mmol, 2 equiv) dropwise at 0° C. The resulting mixture was stirred for 30 min at 0° C. The reaction was quenched with H₂O at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (10:1) to afford 2-acetyl-4-chloro-6-fluorobenzenesulfonyl chloride (1.8 g, 93.2%) as a white solid.

### Step 5: Synthesis of tert-butyl (2S)-2-(2-acetyl-4-chloro-6-fluorobenzenesulfonamido)-3-(6-fluoro-2,3-dimethylphenyl) butanoate

To a stirred solution of tert-butyl (2S)-2-amino-3-(6-fluoro-2,3-dimethylphenyl) butanoate (1.70 g, 6.04 mmol, 1 equiv) in CH₂Cl₂ (20 mL) was added Pyridine (2.44 mL, 30.2 mmol, 5 equiv) dropwise at 0° C. To the above mixture was added 2-acetyl-4-chloro-6-fluorobenzenesulfonyl chloride (1.8 g, 6.64 mmol, 1.1 equiv) in DCM (20 mL) dropwise at 0° C. The resulting mixture was stirred overnight at room temperature. The reaction was quenched with water (20 ml) at room temperature. The resulting mixture was extracted with CH₂Cl₂ (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (10:1) to afford tert-butyl (2S)-2-(2-acetyl-4-chloro-6-fluorobenzenesulfonamido)-3-(6-fluoro-2,3-dimethylphenyl) butanoate (2.5 g, 80.3%). LC-MS: (ES, m/z): [M−H]:514.00

### Step 6: Synthesis of tert-butyl (2S)-2-((2-acetyl-4-chloro-6-hydroxyphenyl) sulfonamido)-3-(6-fluoro-2,3-dimethylphenyl) butanoate

To a solution of 2-methanesulfonylethanol (371 mg, 3 mmol, 2 equiv) in DMF was added sodium hydride (60% in oil, 239 mg) at 0 degrees C. The mixture was stirred for 15 min. tert-butyl (2S)-2-(3-acetyl-4-chloro-2-fluorobenzenesulfonamido)-3-(6-fluoro-2,3-dimethylphenyl) butanoate (770 mg, 1.5 mmol, 1 equiv) was added and the mixture was allowed to warm to RT and stirred overnight at room temperature under nitrogen atmosphere. The reaction was quenched with sat. NH₄Cl (aq., 10 ml) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (8:1) to afford tert-butyl (2S)-2-(3-acetyl-4-chloro-2-hydroxybenzenesulfonamido)-3-(6-fluoro-2,3-dimethylphenyl) butanoate (310 mg, 40.4%).

### Step 7: Synthesis of tert-butyl (2S)-2-(9-acetyl-7-chloro-1,1-dioxido-3,4-dihydro-2H-benzo[b][1,4,5]oxathiazepin-2-yl)-3-(6-fluoro-2,3-dimethylphenyl) butanoate

Into a 40 mL vial were added tert-butyl (2S)-2-(2-acetyl-4-chloro-6-hydroxybenzenesulfonamido)-3-(6-fluoro-2,3-dimethylphenyl) butanoate (310 mg, 0.60 mmol, 1 equiv), DMF (3 mL), dibromoethane (156 μL, 1.9 mmol, 3 equiv) and K₂CO₃ (250 mg, 1.89 mmol, 3 equiv) at room temperature. The resulting mixture was stirred overnight at 65° C. The reaction was quenched with sat. NH₄C1 (aq., 10 ml) at room temperature. The resulting mixture was extracted with EtOAc (3×2 mL). The combined organic layers were washed with brine (3×2 mL), dried over anhydrous Na₂SO₄. After filtration, the filtrate was concentrated under reduced pressure. The residue was purified by silica gel column chromatography, eluted with PE/EtOAc (9:1) to afford tert-butyl (2S)-2-(9-acetyl-7-chloro-1,1-dioxo-3,4-dihydro-5,1lambda6,2-benzoxathiazepin-2-yl)-3-(6-fluoro-2,3-dimethylphenyl) butanoate (270 mg, 82.9%).

### Step 8: Synthesis of (2S)-2-(9-acetyl-7-chloro-1,1-dioxido-3,4-dihydro-2H-benzo[b][1,4,5]oxathiazepin-2-yl)-3-(6-fluoro-2,3-dimethylphenyl)butanoic acid

Into an 8 mL sealed tube were added tert-butyl (2S)-2-(9-acetyl-7-chloro-1,1-dioxo-3,4-dihydro-5,1lambda6,2-benzoxathiazepin-2-yl)-3-(6-fluoro-2,3-dimethylphenyl) butanoate (260 mg, 0.48 mmol, 1 equiv), DCM (2 mL) and trifluoroacetic acid (2 mL) at room temperature. The resulting mixture was stirred 2 h at room temperature. The resulting mixture was concentrated under vacuum. The crude product was used in the next step directly without further purification.

### Step 9: Synthesis of 5-((1S,2R)-1-(9-acetyl-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 8 mL sealed tube were added (2S)-2-(9-acetyl-7-chloro-1,1-dioxido-3,4-dihydro-2H-benzo[b][1,4,5]oxathiazepin-2-yl)-3-(6-fluoro-2,3-dimethylphenyl) butanoic acid (270 mg, 0.56 mmol, 1 equiv), TIF (3 mL) and CDI (226 mg, 1.4 mmol, 2.5 equiv) at room temperature. The resulting mixture was stirred for 40 min at room temperature. To the above mixture was added hydrazine hydrate (244 μL, 5.02 mmol, 9 equiv) dropwise at 0° C. The resulting mixture was stirred for additional 30 min at 0° C. The reaction was quenched with sat. NH₄Cl (aq.) at room temperature. The resulting mixture was extracted with EtOAc (3×1 mL), dried over anhydrous Na₂SO₄. After filtration, the filtrate was concentrated under reduced pressure. To the above mixture was added dioxane (3 mL) and CDI (226.17 mg, 1.4 mmol, 2.5 equiv) at room temperature. The resulting mixture was stirred for 30 min at room temperature. The residue was purified by reversed-phase flash chromatography with the following conditions: Column: XSelect CSH Prep CIS OBD Column, 19*250 mm, 5 μm; Mobile Phase A: Water (0.1% FA), Mobile Phase B: MeOH-HPLC; Flow rate: 20 mL/min; Gradient: 65% B to 80% B in 10 min, 80% B; Wave Length: 220 nm; RT1 (min): 7, 8 (min) to afford 6-acetyl-7-chloro-2-[(1S)-2-(6-fluoro-2,3-dimethylphenyl)-1-(5-oxo-4H-1,3,4-oxadiazol-2-yl)propyl]-3,4-dihydro-5,1lambda6,2-benzoxathiazepine-1,1-dione (78.5 mg, 26.85%). LCMS: (ES, m/z): [M+H]+=524.15. ¹H NMR (300 MHz, DMSO-d₆) δ 12.23 (s, 1H), 7.42-7.41 (d, J=2.1 Hz, 1H), 7.24-7.24 (d, J=2.1 Hz, 1H), 7.05-7.00 (dd, J=8.4, 5.8 Hz, 1H), 6.86-6.79 (dd, J=12.2, 8.4 Hz, 1H), 5.37-5.37 (dd, J=11.8, 2.1 Hz, 1H), 4.67-4.60 (ddd, J=12.8, 8.3, 4.2 Hz, 1H), 4.26-4.21 (d, J=13.5 Hz, 1H), 3.88-3.75 (q, J=13.1, 10.9 Hz, 3H), 2.57 (s, 3H), 2.28 (s, 3H), 2.17 (s, 3H), 2.07 (s, 3H), 1.30-1.28 (d, J=6.7 Hz, 3H).

### Example 61: 5-((1S,2R)-1-(6-chloro-4-methyl-1,1-dioxido-5-(piperidin-1-ylmethyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

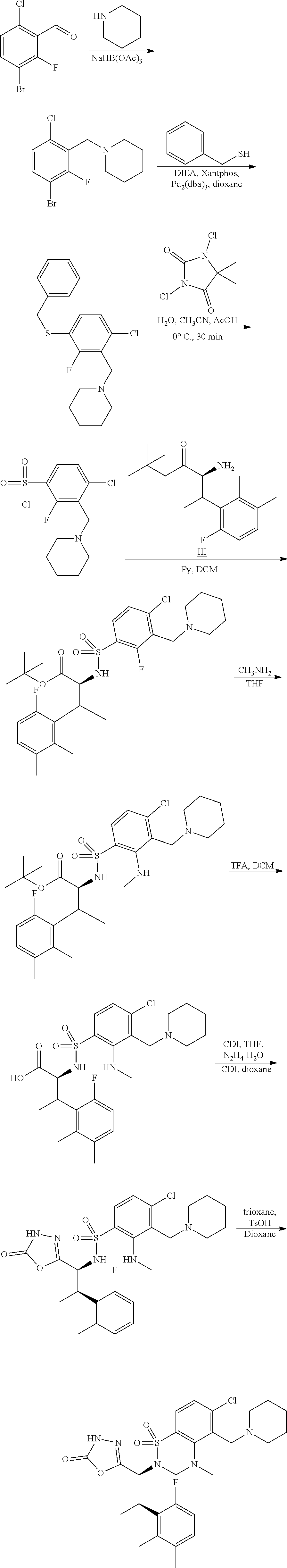

Into a 250 mL round-bottom flask were added 3-bromo-6-chloro-2-fluorobenzaldehyde (1 g, 4.21 mmol, 1 equiv), piperdine (395 mg, 4.6 mmol, 1.1 equiv) and DCM (20 mL) at room temperature. To the above mixture was added Sodium triacetoxyborohydride (1340 mg, 6.32 mmol, 1.5 equiv) in portions at 0° C. The mixture was stirred for additional 1 h at room temperature. The resulting mixture was extracted with EtOAc (2×10 mL). The combined organic layers were washed with brine (1×100 mL), dried over anhydrous Na₂SO₄. After filtration, the filtrate was concentrated under reduced pressure. The residue was purified by silicagel column chromatography, eluted with PE/EtOAc (10:1) to afford 1-[(3-bromo-6-chloro-2-fluorophenyl) methyl]piperidine (700 mg, 54.2%).

### Step 2: Synthesis of 1-(3-(benzylthio)-6-chloro-2-fluorobenzyl) piperidine

Into a 20 mL sealed-tube were added 1-[(3-bromo-6-chloro-2-fluorophenyl) methyl]piperidine (700 mg, 2.28 mmol, 1 equiv), dioxane (14 mL), DIEA (795 L, 4.57 mmol, 2 equiv), Xantphos (132 mg, 0.23 mmol, 0.1 equiv), Pd₂(dba)₃ (105 mg, 0.11 mmol, 0.05 equiv) and benzyl mercaptan (281 μL, 2.4 mmol, 1 equiv) at room temperature. The resulting mixture was stirred overnight at 100° C. under nitrogen atmosphere. The resulting mixture was filtered, the filtercake was washed with EtOAc (3×10 mL). The filtrate was concentrated under reduced pressure. The residue was purified by silicagel column chromatography, eluted with PE/EtOAc (10:1) to afford 1-{[3-(benzylsulfanyl)-6-chloro-2-fluorophenyl]methyl} piperidine (700 mg, 87.6%).

### Step 3: Synthesis of 4-chloro-2-fluoro-3-(piperidin-1-ylmethyl) benzenesulfonyl chloride

### Step 4: Synthesis of tert-butyl (2S)-2-((4-chloro-2-fluoro-3-(piperidin-1-ylmethyl) phenyl) sulfonamido)-3-(6-fluoro-2,3-dimethylphenyl) butanoate

Into a 50 mL vial were added 4-chloro-2-fluoro-3-(piperidin-1-ylmethyl) benzenesulfonyl chloride (290 mg, 0.9 mmol, 1 equiv) and pyridine (2 mL) at room temperature. To the above mixture was added tert-butyl (2S)-2-amino-3-(6-fluoro-2,3-dimethylphenyl) butanoate (12 mg, 0.043 mmol, 0.77 equiv) in ACN dropwise at 0° C. The resulting mixture was stirred for additional 1 h at 0° C. The resulting mixture was quenched with H₂O (30 ml) and extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in tert-butyl (2S)-2-[4-chloro-2-fluoro-3-(piperidin-1-ylmethyl) benzenesulfonamido]-3-(6-fluoro-2,3-dimethylphenyl) butanoate (80 mg, 15.8%).

### Step 5: Synthesis of tert-butyl (2S)-2-((4-chloro-2-(methylamino)-3-(piperidin-1-ylmethyl) phenyl) sulfonamido)-3-(6-fluoro-2,3-dimethylphenyl) butanoate

Into a 10 mL sealed tube were added tert-butyl (2S)-2-[4-chloro-2-fluoro-3-(piperidin-1-ylmethyl) benzenesulfonamido]-3-(6-fluoro-2,3-dimethylphenyl) butanoate (10 mg, 0.018 mmol, 1 equiv), DMSO (3 mL) and CH₃NH₂ (3.6 mL, 7.1 mmol, 15 equiv) at room temperature. The resulting mixture was stirred overnight at 100° C. under nitrogen atmosphere. The residue was purified by reversed-phase flash chromatography with the following conditions: column, C18 silica gel; mobile phase, MeCN in Water (10 mmol/L NH₄HCO₃), 100% gradient in 10 min; detector, UV 254 nm. This resulted in tert-butyl (2S)-2-[4-chloro-2-(methylamino)-3-(piperidin-1-ylmethyl) benzenesulfonamido]-3-(6-fluoro-2,3-dimethylphenyl) butanoate (257 mg, 93.4%).

### Step 6: Synthesis of (2S)-2-((4-chloro-2-(methylamino)-3-(piperidin-1-ylmethyl) phenyl) sulfonamido)-3-(6-fluoro-2,3-dimethylphenyl) butanoic acid

Into a 50 mL round-bottom flask were added tert-butyl (2S)-2-[4-chloro-2-(methylamino)-3-(piperidin-1-ylmethyl) benzenesulfonamido]-3-(6-fluoro-2,3-dimethylphenyl) butanoate (100 mg, 0.17 mmol, 1 equiv), DCM (1 mL) and TFA (3 mL) at room temperature. The resulting mixture was stirred overnight at room temperature under air atmosphere. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in (2S)-2-[4-chloro-2-(methylamino)-3-(piperidin-1-ylmethyl) benzenesulfonamido]-3-(6-fluoro-2,3-dimethylphenyl) butanoic acid (46 mg, 50.9%).

### Step 7: Synthesis of 4-chloro-N-((1S,2R)-2-(6-fluoro-2,3-dimethylphenyl)-1-(5-oxo-4,5-dihydro-1,3,4-oxadiazol-2-yl)propyl)-2-(methylamino)-3-(piperidin-1-ylmethyl)benzenesulfonamide

Into a 10 mL vial were added (2S)-2-[4-chloro-2-(methylamino)-3-(piperidin-1-ylmethyl) benzenesulfonamido]-3-(6-fluoro-2,3-dimethylphenyl) butanoic acid (120 mg, 0.23 mmol, 1 equiv), CDI (55.5 mg, 0.34 mmol, 1.5 equiv) and THF (2.40 mL) at room temperature. The resulting mixture was stirred for 50 min at room temperature under air atmosphere. To the above mixture was added NH₂NH₂·H₂O (55.4 μL, 1.14 mmol, 5 equiv) dropwise at 0° C. The resulting mixture was stirred for additional 30 min at 0° C. The resulting mixture was extracted with EtOAc (2×30 mL), dried over anhydrous Na₂SO₄. After filtration, the filtrate was concentrated under reduced pressure. Into a 10 mL vial were added the above crude product, dioxane (2.40 mL) and CDI (148 mg, 0.91 mmol, 4 equiv) at room temperature. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 4-chloro-N-[(1S,2R)-2-(6-fluoro-2,3-dimethylphenyl)-1-(5-oxo-4H-1,3,4-oxadiazol-2-yl) propyl]-2-(methylamino)-3-(piperidin-1-ylmethyl) benzenesulfonamide (80 mg, 62%).

### Step 8: Synthesis of 5-((1S,2R)-1-(6-chloro-4-methyl-1,1-dioxido-5-(piperidin-1-ylmethyl)-3,4-dihydro-2H-benzo[e][1,2,4]thiadiazin-2-yl)-2-(6-fluoro-2,3-dimethylphenyl) propyl)-1,3,4-oxadiazol-2 (3H)-one

Into a 10 mL sealed tube were added 4-chloro-N-[(1S,2R)-2-(6-fluoro-2,3-dimethylphenyl)-1-(5-oxo-4H-1,3,4-oxadiazol-2-yl)propyl]-2-(methylamino)-3-(piperidin-1-ylmethyl)benzenesulfonamide (57 mg, 0.10 mmol, 1 equiv), dioxane (1 mL), trioxane (90.7 mg, 1.01 mmol, 10 equiv) and TsOH (26 mg, 0.15 mmol, 1.5 equiv) at room temperature. The resulting mixture was stirred overnight at 100° C. under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 30% to 50% gradient in 10 min; detector, UV 254 nm. This resulted in of 5-((1S,2R)-1-(6-chloro-4-methyl-1,1-dioxido-5-(piperidin-1-ylmethyl)-3,4-dihydro-2H-benzo[e][1,2,4]thiadiazin-2-yl)-2-(6-fluoro-2,3-dimethylphenyl) propyl)-1,3,4-oxadiazol-2 (3H)-one (17.3 mg, 29.7%). LCMS: (ES, m/z):[M+H]+=578.15. ¹H NMR (300 MHz, Methanol-d₄) δ 7.90-7.68 (m, 1H), 7.37-7.21 (m, 1H), 7.09-6.94 (m, 1H), 6.79-6.59 (m, 1H), 4.87-4.58 (m, 5H), 3.77-3.61 (m, 2H), 3.56-3.42 (m, 3H), 3.26 (s, 2H), 2.39 (s, 1H), 2.35-2.10 (m, 6H), 2.04 (s, 4H), 1.83 (s, 2H), 1.54 (d, J=6.9 Hz, 2H), 1.17 (d, J=7.1 Hz, 1H).

### Example 62: 5-((1S,2R)-1-(6-chloro-1,1-dioxido-4-(((S)-tetrahydrofuran-2-yl) methyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for Ex-30 using (3R)-1-methylpyrrolidin-3-amine instead of 2-methoxypropylamine to obtain 5-((1S,2R)-1-(6-chloro-1,1-dioxido-4-(((S)-tetrahydrofuran-2-yl)methyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.018 g, yield 33%). ¹H NMR (400 MHz, DMSO-d₆) δ 12.05 (s, 1H), 7.55 (d, J=8.5 Hz, 1H), 7.02 (dd, J=8.4, 5.8 Hz, 1H), 6.93 (d, J=1.9 Hz, 1H), 6.86-6.80 (m, 1H), 6.78 (dd, J=8.4, 1.8 Hz, 1H), 5.32 (d, J=15.2 Hz, 1H), 5.30-5.22 (m, 2H), 4.02 (dq, J=9.8, 3.7, 2.9 Hz, 1H), 3.99-3.90 (m, 1H), 3.77 (dt, J=8.2, 6.8 Hz, 1H), 3.64 (td, J=7.9, 5.7 Hz, 1H), 3.55 (dd, J=15.3, 7.0 Hz, 1H), 3.27 (dd, J=15.5, 3.9 Hz, 1H), 2.31 (s, 3H), 2.17 (s, 3H), 2.02-1.74 (m, 3H), 1.54-1.43 (m, 1H), 1.33 (d, J=6.8 Hz, 3H). LC-MS: m/z=[M−H]+=551.23.

### Example 63: 5-((1S,2R)-1-(6-chloro-4-(2-methoxyethyl)-1,1-dioxido-3,4-dihydro-2H-pyrido[2,3-e][1,2,4]thiadiazin-2-yl)-2-(6-fluoro-2,3-dimethylphenyl) propyl)-1,3,4-oxadiazol-2 (3H)-one

The title compound was synthesized following the procedure described in Ex-30 using 2-methoxyethylamine instead of 3-methoxypropylamine and (2S,3R)-2-(6-chloro-2-fluoropyridine-3-sulfonamido)-3-(6-fluoro-2,3-dimethylphenyl)butanoic acid (Pyridine core) instead of (2S,3R)-2-(4-chloro-2-fluorobenzenesulfonamido)-3-(6-fluoro-2,3-dimethylphenyl)butanoic acid (Phenyl core) to obtain 5-((1S,2R)-1-(6-chloro-4-(2-methoxyethyl)-1,1-dioxido-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one (0.015 g, 0.029 mmol, yield 47%). ¹H NMR (400 MHz, Methanol-d₄) δ 7.90 (d, J=8.0 Hz, 1H), 7.00 (dd, J=8.4, 5.7 Hz, 1H), 6.78 (d, J=8.0 Hz, 1H), 6.72 (dd, J=12.1, 8.4 Hz, 1H), 5.46 (d, J=15.0 Hz, 1H), 5.41 (dd, J=12.0, 1.8 Hz, 1H), 5.17 (d, J=15.0 Hz, 1H), 4.18 (dt, J=14.6, 3.7 Hz, 1H), 3.98-3.88 (m, 1H), 3.62-3.51 (m, 2H), 3.37 (s, 3H), 3.24 (ddd, J=14.6, 8.5, 4.1 Hz, 1H), 2.38 (s, 3H), 2.23 (s, 3H), 1.47-1.43 (m, 3H). LC-MS: m/z=[M−H]+=526.19.

### Example 64: 5-((1S,2R)-1-(5-acetyl-6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

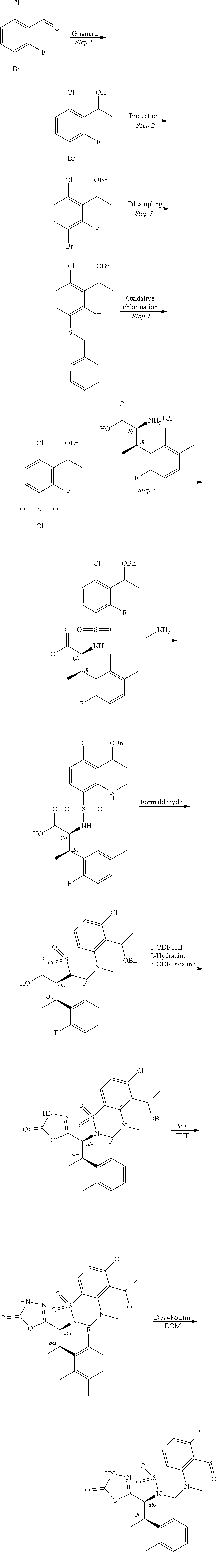

### Step 1: Synthesis of 1-(3-bromo-6-chloro-2-fluorophenyl) ethan-1-ol

3-bromo-6-chloro-2-fluorobenzaldehyde (1.87 g, 7.89 mmol, 1.0 eq) was added to a dry degassed flask in anhydrous THF (20 mL) and was cooled in an ice bath while stirring. To this, 3.0 M solution of MeMgBr (4.6 mL, 1.75 eq) was added dropwise while stirring. The solution was continued to stir in the ice bath for 1 hour and then the temperature was increased to room temperature with stirring for another 30 minutes. Upon completion of the reaction, the solution was quenched with sat. NH₄Cl solution and extracted with EtOAc. The organics were combined, washed with brine, and then dried over MgSO₄. The solvent was removed under vacuum to obtain 1-(3-bromo-6-chloro-2-fluorophenyl) ethan-1-ol (2.0 g, yield 100%).

### Step 2: Synthesis of 2-(1-(benzyloxy) ethyl)-4-bromo-1-chloro-3-fluorobenzene

1-(3-bromo-6-chloro-2-fluorophenyl) ethan-1-ol (1.95 g, 1.0 eq) was dissolved in anhydrous THF (16.0 mL) and the solution was cooled in an ice bath. To this, sodium hydride 60% (0.46 g, 1.5 eq) was added portion wise while stirring. Next, tetrabutylammonium iodide (50 mg) solution of BnBr in 4 mL THF, (1.1 eq) was added to the reaction mixture while continuing to stir in the ice bath for 30 minutes and then for a further 2.5 h at room temperature. Upon completion, the reaction was quenched with sat. NH₄Cl and extracted with diethyl ether. The organics were combined, washed with brine, and dried over MgSO₄. The solvent was evaporated to obtain a crude colorless oil, which was further purified by column chromatography (CC) using 5% EtOAc in Hexanes to obtain the final product 2-[1-(benzyloxy) ethyl]-4-bromo-1-chloro-3-fluorobenzene (2.35 g, 89%).

### Step 3: Synthesis of (2S,3R)-2-((3-(1-(benzyloxy) ethyl)-4-chloro-2-fluorophenyl) sulfonamido)-3-(6-fluoro-2,3-dimethylphenyl) butanoic acid

The benzyloxyacetyl core (2S,3R)-2-{3-[1-(benzyloxy) ethyl]-4-chloro-2-fluorobenzenesulfonamido}-3-(6-fluoro-2,3-dimethylphenyl) butanoic acid (0.8 g, yield 61%) was obtained according to the usual procedure described in Ex 2 steps 1, 2 and 3.

### Step 4: Synthesis of 5-((1S,2R)-1-(5-acetyl-6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The oxadiazolone synthesis was carried out following procedure described for Ex-30 step 2,3 and 4 using methylamine solution in THF instead of 2-methoxypropylamine and (2S,3R)-2-{3-[1-(benzyloxy)ethyl]-4-chloro-2-fluorobenzenesulfonamido}-3-(6-fluoro-2,3-dimethylphenyl)butanoic acid (benzyloxyacetyl core). The product obtained was dissolved in THF (3 mL), degassed, and purged with argon while stirring at room temperature. Next, Pd (10% on carbon) (0.02 eq) was slowly added. The solution was continued to stir for another 1 hour until completion. The solution was passed through celite and the filtrate was concentrated under vacuum to obtain 5-((1S,2R)-1-(6-chloro-5-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.016 g, yield 99%) which was directly used in the subsequent step.

5-((1S,2R)-1-(6-chloro-5-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (16.0 mg, 1.0 eq) was dissolved in DCM (1.2 mL) and to this, Dess-Martin periodinane, 95% (30.0 mg, 2.5 eq) was added portion wise. Upon completion, the reaction mixture was filtered through celite and purified by CC (DCM:EtOAc 20:1) to obtain of 5-((1S,2R)-1-(5-acetyl-6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.012 g, yield 74%). ¹H NMR (300 MHz, Methanol-d₄) δ 7.72-7.64 (m, 1H), 7.04-6.94 (m, 2H), 6.73 (t, J=10.0 Hz, 1H), 5.42 (d, J=11.8 Hz, 1H), 5.18 (d, J=14.1 Hz, 1H), 5.07-4.97 (m, 1H), 3.95-3.81 (m, 1H), 2.88-2.78 (m, 3H), 2.59-2.51 (m, 3H), 2.36 (s, 3H), 2.22 (s, 3H), 1.46 (d, J=6.8 Hz, 3H). LC-MS: m/z=[M−H]+=523.20.

### Example 65: 5-((1S,2R)-1-(6-chloro-4-(1-methylazetidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The previously synthesized 5-((1S,2R)-1-(4-(azetidin-3-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one from Ex 57, (0.026 g, 0.049 mmol, 1.0 eq) was dissolved in methanol (0.49 ml, 0.1 M) and N,N-diisopropylethylamine (1.1 eq) was added, along with sodium acetate (0.02 g, 0.24 mmol, 5eq) and formaldehyde 30% aq solution (20 eq) with stirring. The solution was stirred at room temperature for 1 hour and then, sodium cyanoborohydride (0.015 g, 0.244 mmol, 5.0 eq) was added. The reaction mixture was continued to stir for another 1 hour at room temperature. Upon completion of the reaction, the mixture was quenched with sat. NaHCO₃ and extracted with EtOAc. The organics were combined, washed with brine, filtered, and concentrated under reduced pressure to dryness. The residue was pre-purified by prep TLC with 50% EtOAc in Hexanes and then further purified by 5% MeOH in DCM to obtain the title compound 5-((1S,2R)-1-(6-chloro-4-(1-methylazetidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.0044 g, yield 16%). ¹H NMR (400 MHz, Methanol-d₄) δ7.57 (d, J=8.4 Hz, 1H), 6.91 (dd, J=8.4, 5.7 Hz, 1H), 6.79 (dd, J=8.4, 1.8 Hz, 1H), 6.64 (dd, J=12.0, 8.3 Hz, 1H), 6.46 (d, J=1.8 Hz, 1H), 5.45 (d, J=11.7 Hz, 1H), 5.30 (d, J=14.1 Hz, 1H), 5.15 (d, J=14.1 Hz, 1H), 4.07 (p, J=7.0 Hz, 1H), 3.92-3.82 (m, 2H), 3.77-3.71 (m, 1H), 3.52-3.46 (m, 1H), 3.15-3.09 (m, 1H), 2.43 (s, 3H), 2.39 (s, 3H), 2.20 (s, 3H), 1.45 (dd, J=7.0, 1.1 Hz, 3H). LC-MS: m/z=[M−H]+=536.04.

### Example 66: 5-((1S,2R)-2-(6-fluoro-2,3-dimethylphenyl)-1-(6-hydroxy-4-methyl-1,1-dioxido-3,4-dihydro-2H-benzo[e][1,2,4]thiadiazin-2-yl)propyl)-1,3,4-oxadiazol-2 (3H)-one

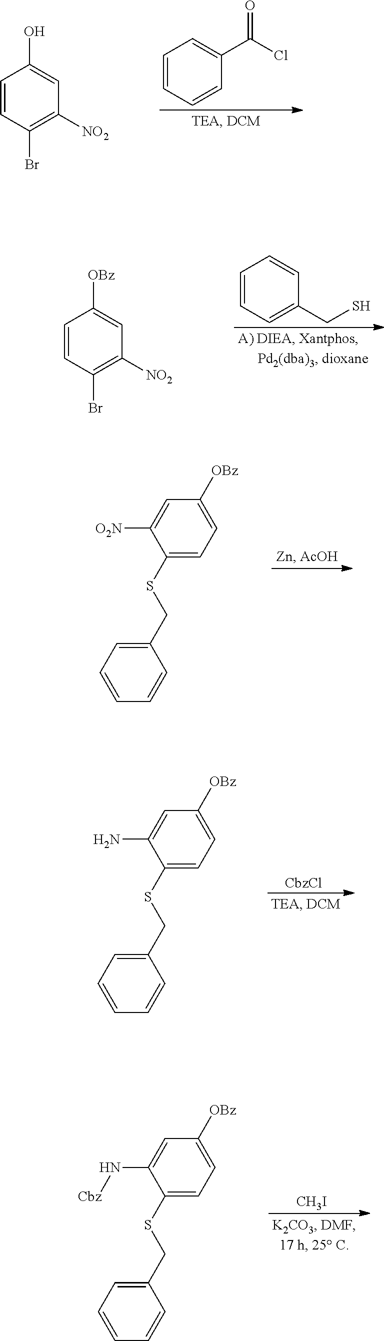

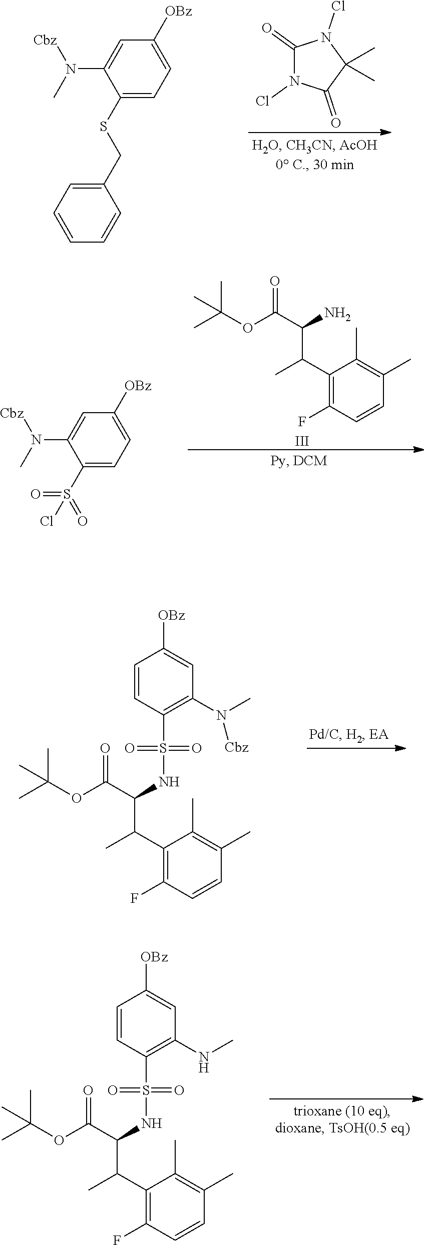

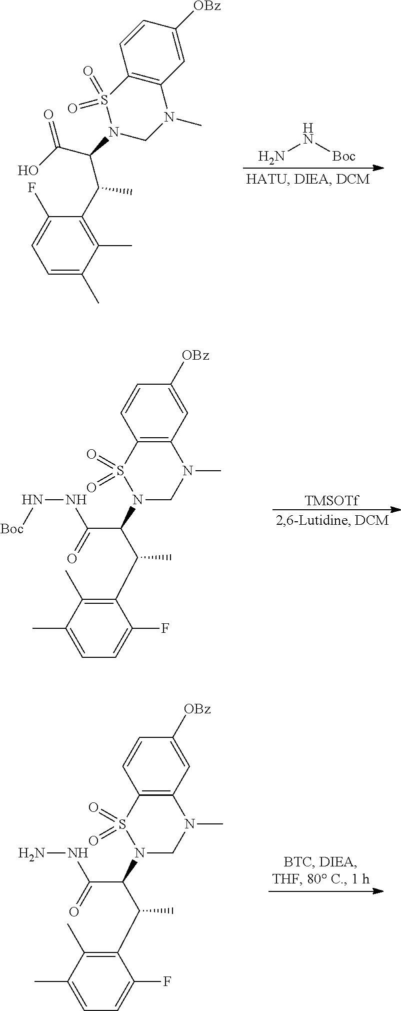

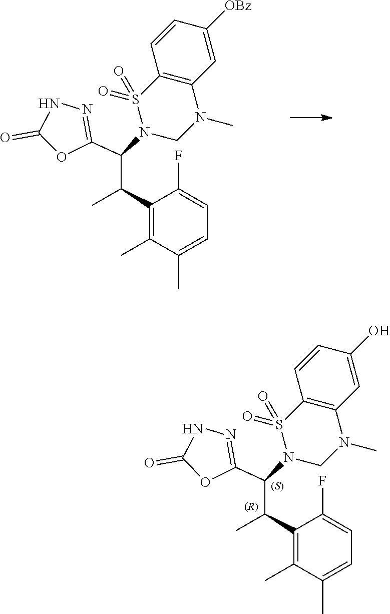

### Step 1: Synthesis of 4-bromo-3-nitrophenyl benzoate

Into a 40 mL sealed tube were added 4-bromo-3-nitrophenol (1 g, 4.59 mmol, 1 equiv), DCM (20 mL), TEA (1.28 mL, 9.17 mmol, 2 equiv). To the mixture benzoyl chloride (2.66 mL, 23 mmol, 5 equiv) at 0° C. The resulting mixture was stirred for 30 min at 0° C. under air atmosphere. The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 4-bromo-3-nitrophenyl benzoate (1.4 g, 93.9%).

### Step 2: Synthesis of 4-(benzylthio)-3-nitrophenyl benzoate

Into a 20 mL sealed tube were added 4-bromo-3-nitrophenyl benzoate (200 mg, 0.62 mmol, 1 equiv), Dioxane (4 mL), DIEA (324 μL, 1.86 mmol, 3 equiv), Xantphos (72 mg, 0.12 mmol, 0.2 equiv), benzyl mercaptan (73 μL, 0.62 mmol, 1 equiv) and Pd₂(dba)₃ (56.9 mg, 0.06 mmol, 0.1 equiv) at room temperature. The resulting mixture was stirred for overnight at 100° C. under nitrogen atmosphere. The resulting mixture was filtered, the filter cake was washed with ethyl acetate (3×10 mL). The filtrate was concentrated under reduced pressure. The residue was purified by Prep-TLC (PE/EtOAc 5:1) to afford 4-(benzylthio)-3-nitrophenyl benzoate (210 mg, 92.56%) Step 3: Synthesis of 3-amino-4-(benzylthio)phenyl benzoate

Into an 8 mL sealed tube were added 4-(benzylthio)-3-nitrophenyl benzoate (250 mg, 0.68 mmol, 1 equiv), AcOH (2.5 mL) and Zn (671 mg, 10.3 mmol, 15 equiv) at room temperature. The resulting mixture was stirred for 15 min at room temperature under air atmosphere. The resulting mixture was filtered, the filter cake was washed with ethyl acetate (3×10 mL). The filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 3-amino-4-(benzylthio) phenyl benzoate (165 mg, 71.9%).

### Step 4: Synthesis of 3-(((benzyloxy)carbonyl) amino)-4-(benzylthio)phenyl benzoate

Into a 40 mL sealed tube were added 3-amino-4-(benzylthiol) phenyl benzoate (700 mg, 2.09 mmol, 1 equiv), THF (7 mL), H₂O (7 mL), Na2CO3 (442 mg, 4.17 mmol, 2 equiv) and Cbzcl (1.47 mL, 10.4 mmol, 5 equiv) at 0° C. The resulting mixture was stirred for 2 h at room temperature under nitrogen atmosphere. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 3-{[(benzyloxy) carbonyl]amino}-4-(benzylthio) phenyl benzoate (953 mg, 98%).

### Step 5: Synthesis of 3-(((benzyloxy)carbonyl)(methyl)amino)-4-(benzylthio)phenyl benzoate

Into a 100 mL round-bottom flask were added 3-{[(benzyloxy) carbonyl]amino}-4-(benzylsulfanyl) phenyl benzoate (983 mg, 2.09 mmol, 1 equiv), DMF (30 mL), K₂CO₃ (579 mg, 4.19 mmol, 2 equiv). To the mixture was added CH₃I (130 μL, 2.09 mmol, 1 equiv) at 0° C. The resulting mixture was stirred for overnight at room temperature under air atmosphere. The resulting mixture was extracted with EtOAc (3×10 mL). The combined organic layers were washed with brine (3×10 mL), dried over anhydrous Na₂SO₄.

After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 3-{[(benzyloxy)carbonyl](methyl)amino}-4-(benzylsulfanyl) phenyl benzoate (803 mg, 79.3%).

### Step 6: Synthesis of 3-(((benzyloxy)carbonyl) (methyl)amino)-4-(chlorosulfonyl)phenyl benzoate

Into an 8 mL sealed tube were added 3-{[(benzyloxy) carbonyl](methyl)amino}-4-(benzylsulfanyl) phenyl benzoate (252 mg, 0.52 mmol, 1 equiv), ACN (5.04 mL), AcOH (131 μL), H₂O 94 μL). To the mixture was added 1,3-dichloro-5,5-dimethylimidazolidine-2,4-dione (205 mg, 1.04 mmol, 2 equiv) at 0° C.

The resulting mixture was stirred for 30 min at 0° C. under air atmosphere. The resulting mixture was quenched with water and extracted with EtOAc (3×10 mL). The combined organic layers were washed with brine (3×10 mL), dried over anhydrous Na₂SO₄. After filtration, the filtrate was concentrated under reduced pressure. This resulted in 3-{[(benzyloxy) carbonyl](methyl)amino}-4-(chlorosulfonyl) phenyl benzoate (409 mg).

### Step 7: Synthesis of 3-(((benzyloxy)carbonyl) (methyl)amino)-4-(N-((2S)-1-(tert-butoxy)-3-(6-fluoro-2,3-dimethylphenyl)-1-oxobutan-2-yl) sulfamoyl) phenyl benzoate

Into a 100 mL round-bottom flask were added tert-butyl (2S)-2-amino-3-(6-fluoro-2,3-dimethylphenyl) butanoate (410 mg, 1.46 mmol, 1.00 equiv) and Pyridine (10 mL). To the mixture was added 3-{[(benzyloxy) carbonyl](methyl)amino}-4-(chlorosulfonyl) phenyl benzoate (1.01 g, 2.19 mmol, 1.5 equiv) at 0° C. The resulting mixture was stirred for overnight at room temperature under air atmosphere.

The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 3-{[(benzyloxy)carbonyl](methyl)amino}-4-{[(2S)-1-(tert-butoxy)-3-(6-fluoro-2,3-dimethylphenyl)-1-oxobutan-2-yl]sulfamoyl} phenyl benzoate (695 mg, 67.7%).

### Step 8: Synthesis of 4-(N-((2S)-1-(tert-butoxy)-3-(6-fluoro-2,3-dimethylphenyl)-1-oxobutan-2-yl) sulfamoyl)-3-(methylamino)phenyl benzoate

Into a 100 mL round-bottom flask were added 3-{[(benzyloxy) carbonyl](methyl)amino}-4-{[(2S)-1-(tert-butoxy)-3-(6-fluoro-2,3-dimethylphenyl)-1-oxobutan-2-yl]sulfamoyl} phenyl benzoate (633 mg, 0.9 mmol, 1 equiv), EA (18 mL) and Pd/C (633 mg, 6 mmol, 6.6 equiv) at room temperature. The resulting mixture was stirred for 1 h at room temperature under hydrogen atmosphere. The resulting mixture was filtered, the filter cake was washed with ethyl acetate (3×10 mL). The filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 4-{[(2S)-1-(tert-butoxy)-3-(6-fluoro-2,3-dimethylphenyl)-1-oxobutan-2-yl]sulfamoyl}-3-(methylamino) phenyl benzoate (493 mg, 96.2%).

### Step 9: Synthesis of (2S,3R)-2-(6-(benzoyloxy)-4-methyl-1,1-dioxido-3,4-dihydro-2H-benzo[e][1,2,4]thiadiazin-2-yl)-3-(6-fluoro-2,3-dimethylphenyl)butanoic acid

Into an 8 mL sealed tube were added 4-{[(2S)-1-(tert-butoxy)-3-(6-fluoro-2,3-dimethylphenyl)-1-oxobutan-2-yl]sulfamoyl}-3-(methylamino) phenyl benzoate (493 mg, 0.86 mmol, 1 equiv), Dioxane (6 mL), 1,3,5-trioxane (778 mg, 8.64 mmol, 10 equiv) and TsOH (74.4 mg, 0.43 mmol, 0.5 equiv) at room temperature and stirred overnight at 100° C. The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in (2S,3R)-2-[6-(benzoyloxy)-4-methyl-1,1-dioxo-3H-1lambda6,2,4-benzothiadiazin-2-yl]-3-(6-fluoro-2,3-dimethylphenyl) butanoic acid (306 mg, 67.3%).

### Step 10: Synthesis of 2-((1S,2R)-2-(6-fluoro-2,3-dimethylphenyl)-1-(5-oxo-4,5-dihydro-1,3,4-oxadiazol-2-yl) propyl)-4-methyl-1,1-dioxido-3,4-dihydro-2H-benzo[e][1,2,4]thiadiazin-6-yl benzoate

The oxadiazolone formation was done using the 3 step synthesis described in Ex 2 resulting in 2-((1S,2R)-2-(6-fluoro-2,3-dimethylphenyl)-1-(5-oxo-4,5-dihydro-1,3,4-oxadiazol-2-yl) propyl)-4-methyl-1,1-dioxido-3,4-dihydro-2H-benzo[e][1,2,4]thiadiazin-6-yl benzoate (215 mg, 69.2%).

### Step 11: Synthesis of 5-((1S,2R)-2-(6-fluoro-2,3-dimethylphenyl)-1-(6-hydroxy-4-methyl-1,1-dioxido-3,4-dihydro-2H-benzo[e][1,2,4]thiadiazin-2-yl) propyl)-1,3,4-oxadiazol-2 (3H)-one

Into a 20 mL vial were added in 2-((1S,2R)-2-(6-fluoro-2,3-dimethylphenyl)-1-(5-oxo-4,5-dihydro-1,3,4-oxadiazol-2-yl) propyl)-4-methyl-1,1-dioxido-3,4-dihydro-2H-benzo[e][1,2,4]thiadiazin-6-yl benzoate (146 mg, 0.26 mmol, 1 equiv), DMF (5 mL), H₂O (1.7 mL) and K₂CO₃ (54 mg, 0.39 mmol, 1.5 equiv) at room temperature. The resulting mixture was stirred for 2 h at 60° C. under air atmosphere. The resulting mixture was concentrated under vacuum. The residue was purified by reversed-phase flash chromatography with the following conditions: column, C18 silica gel; mobile phase, MeCN in Water (10 mmol/L NH₄HCO₃), 0% to 100% gradient in 20 min; detector, UV 254 nm. The crude product was purified by Chiral-Prep-HPLC with the following conditions: Column, YMC-Actus Triart C18 ExRS, 30*150 mm, 5 μm; mobile phase, 10 mmol NH₄HCO₃+0.05% NH₃H₂O and ACN (19% ACN up to 49% in 8 min); Detector, uv 254&220 nm. This resulted in of 5-((1S,2R)-2-(6-fluoro-2,3-dimethylphenyl)-1-(6-hydroxy-4-methyl-1,1-dioxido-3,4-dihydro-2H-benzo[e][1,2,4]thiadiazin-2-yl) propyl)-1,3,4-oxadiazol-2 (3H)-one (6.2 mg, 4.7%). LCMS:(ES, m/z): [M+H]:461.05. ¹H NMR (300 MHz, DMSO-d₆) δ 7.35 (d, J=8.6 Hz, 1H), 7.00 (dd, J=8.5, 5.8 Hz, 1H), 6.80 (dd, J=12.2, 8.4 Hz, 1H), 6.20 (dd, J=8.6, 2.1 Hz, 1H), 5.92 (d, J=2.2 Hz, 1H), 5.37-4.77 (m, 3H), 3.91-3.78 (m, 1H), 2.73 (s, 3H), 2.30 (s, 3H), 2.16 (s, 3H), 1.32 (d, J=6.7 Hz, 3H).

### Example 67: 5-((1S,2R)-1-(6-chloro-4-(methylsulfonyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized according to example 56 using methylsulfonyl chloride instead of acetyl chloride resulting in 5-((1S,2R)-1-(6-chloro-4-(methylsulfonyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one(17 mg, 73%). ¹H NMR (300 MHz, Methanol-d₄) δ 7.58-7.52 (m, 1H), 7.02-6.98 (m, 1H), 6.86-6.76 (m, 3H), 5.42 (d, J=11.8 Hz, 1H), 5.30 (d, J=14.1 Hz, 1H), 5.15-5.10 (m, 2H), 3.95-3.90 (m, 1H), 2.50 (s, 3H), 2.33 (s, 3H), 2.18 (s, 3H), 1.36-1.35 (d, J=6.8 Hz, 3H).

### Example 68: 5-((1S,2R)-1-(6-chloro-8-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

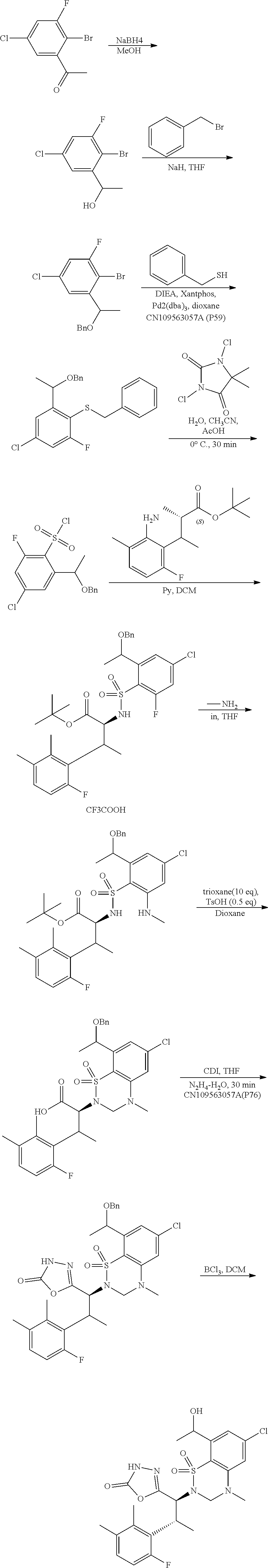

### Step 1: Synthesis of 1-(2-bromo-5-chloro-3-fluorophenyl) ethan-1-ol

To a stirred solution of 1-(2-bromo-5-chloro-3-fluorophenyl) ethanone (1.9 g, 7.6 mmol, 1 equiv) in MeOH was added NaBH₄ (572 mg, 15.1 mmol, 2 equiv) in portions at room temperature. The resulting mixture was stirred for 30 min at room temperature. The reaction was quenched with water (50 ml) at room temperature. The resulting mixture was extracted with EtOAc (3×50 mL). The combined organic layers were washed with brine (1×100 mL), dried over anhydrous Na₂SO₄. After filtration, the filtrate was concentrated under reduced pressure. The crude product was used in the next step directly without further purification.

### Step 2: Synthesis of 1-(1-(benzyloxy) ethyl)-2-bromo-5-chloro-3-fluorobenzene

To a stirred solution of 1-(2-bromo-5-chloro-3-fluorophenyl) ethanol (1.93 g, 7.6 mmol, 1 equiv) in DMF (15 ml) was added NaH (457 mg, 11.4 mmol, 1.5 equiv, 60%) in portions at 0° C. under nitrogen atmosphere. The resulting mixture was stirred for 30 min at room temperature under nitrogen atmosphere.

To the above mixture was added benzyl bromide (1.09 mL, 9.1 mmol, 1.2 equiv) dropwise at 0° C. The resulting mixture was stirred for additional 2 h at room temperature. The reaction was quenched with water (50 ml) at room temperature. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EtOAc (10:1) to afford 1-[1-(benzyloxy) ethyl]-2-bromo-5-chloro-3-fluorobenzene (2.5 g, 95.6%).

### Step 3: Synthesis of benzyl(2-(1-(benzyloxy) ethyl)-4-chloro-6-fluorophenyl)sulfane

To a stirred solution of 1-[1-(benzyloxy) ethyl]-2-bromo-5-chloro-3-fluorobenzene (2.5 g, 7.3 mmol, 1 equiv) and DIEA (3800 μL, 21.8 mmol, 3 equiv) in 1,4-dioxane were added Xantphos (842 mg, 1.5 mmol, 0.2 equiv) and Pd₂(dba)₃ (666 mg, 0.73 mmol, 0.1 equiv) at room temperature under nitrogen atmosphere. To the above mixture was added benzyl mercaptan (1037 μL, 8.73 mmol, 1.2 equiv) at room temperature. The resulting mixture was stirred for additional 3 h at 110° C. The mixture was allowed to cool down to room temperature. The resulting mixture was filtered, the filter cake was washed with EtOAc (2×2 mL). The filtrate was concentrated under reduced pressure. The residue was purified by silica gel column chromatography, eluted with PE/EtOAc (10:1) to afford 1-[1-(benzyloxy) ethyl]-2-(benzylsulfanyl)-5-chloro-3-fluorobenzene (521 mg, 18.5%).

### Step 4: Synthesis of 2-(1-(benzyloxy) ethyl)-4-chloro-6-fluorobenzenesulfonyl chloride

To a stirred mixture of 1-[1-(benzyloxy) ethyl]-2-(benzylsulfanyl)-5-chloro-3-fluorobenzene (510 mg, 1.32 mmol, 1 equiv) and H₂O (0.2 mL) in acetonitrile was added AcOH (378 μL) at 0° C. To the above mixture was added 1,3-dichloro-5,5-dimethylimidazolidine-2,4-dione (519 mg, 2.6 mmol, 2 equiv) in portions at 0° C. The resulting mixture was stirred for additional 30 min at 0° C. The reaction was quenched with Water/Ice (30 ml) at 0° C. The resulting mixture was extracted with EtOAc (2×20 mL). The combined organic layers were washed with brine (1×50 mL), dried over anhydrous Na₂SO₄. After filtration, the filtrate was concentrated under reduced pressure. The crude product was used in the next step directly without further purification.

### Step 5: Synthesis of tert-butyl (2S)-2-((2-(1-(benzyloxy) ethyl)-4-chloro-6-fluorophenyl) sulfonamido)-3-(6-fluoro-2,3-dimethylphenyl) butanoate

To a stirred solution of tert-butyl (2S)-2-amino-3-(6-fluoro-2,3-dimethylphenyl) butanoate (300 mg, 1.07 mmol, 1 equiv) and Pyridine (422 mg, 5.3 mmol, 5 equiv) in DCM was added 2-[1-(benzyloxy) ethyl]-4-chloro-6-fluorobenzenesulfonyl chloride (387 mg, 1.07 mmol, 1 equiv) in portions at 0° C. The resulting mixture was stirred for 2 h at room temperature. The resulting mixture was concentrated under vacuum. The residue was purified by reversed-phase flash chromatography with the following conditions: column, C18 silica gel; mobile phase, MeCN in Water (0.1% FA), 10% to 100% gradient in 10 min; detector, UV 254 nm. This resulted in tert-butyl (2S)-2-{2-[1-(benzyloxy) ethyl]-4-chloro-6-fluorobenzenesulfonamido}-3-(6-fluoro-2,3-dimethylphenyl) butanoate (255 mg, 84.6%).

### Step 6: Synthesis of tert-butyl (2S)-2-((2-(1-(benzyloxy) ethyl)-4-chloro-6-(methylamino) phenyl) sulfonamido)-3-(6-fluoro-2,3-dimethylphenyl) butanoate

To a stirred solution of tert-butyl (2S)-2-{2-[1-(benzyloxy) ethyl]-4-chloro-6-fluorobenzenesulfonamido}-3-(6-fluoro-2,3-dimethylphenyl) butanoate (257 mg, 0.42 mmol, 1 equiv) in THF was added methylamine (3170 μL, 6.4 mmol, 15 equiv) dropwise at room temperature. To the above mixture was added TEA (881 μL, 6.4 mmol, 15 equiv) at room temperature. The resulting mixture was stirred overnight at 65° C. The residue was purified by reversed-phase flash chromatography with the following conditions: column, C18 silica gel; mobile phase, MeCN in Water (0.1% FA), 10% to 100% gradient in 10 min; detector, UV 254 nm. This resulted (2S)-2-((2-(1-(benzyloxy)ethyl)-4-chloro-6-(methylamino)phenyl)sulfonamido)-3-(6-fluoro-2,3-dimethylphenyl)butanoate (231 mg, 88.3%).

### Step 7: Synthesis of (2S)-2-(8-(1-(benzyloxy) ethyl)-6-chloro-4-methyl-1,1-dioxido-3,4-dihydro-2H-benzo[e][1,2,4]thiadiazin-2-yl)-3-(6-fluoro-2,3-dimethylphenyl) butanoic acid

To a stirred solution of (2S)-2-((2-(1-(benzyloxy) ethyl)-4-chloro-6-(methylamino) phenyl) sulfonamido)-3-(6-fluoro-2,3-dimethylphenyl) butanoate (231 mg, 0.032 mmol, 1 equiv) and TsOH (5.56 mg, 0.032 mmol, 1 equiv) in 1,4-dioxane was added 1,3,5-trioxane (43.6 mg, 0.48 mmol, 15 equiv) dropwise at room temperature. The resulting mixture was stirred overnight at 110° C. The resulting mixture was concentrated under vacuum. The resulting mixture was diluted with DCM (2 mL). To the above mixture was added TFA (1.25 mL, 16.8 mmol, 45 equiv) at room temperature. The resulting mixture was stirred for additional 1 h at room temperature. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 NM. This resulted in (2S)-2-(8-(1-(benzyloxy) ethyl)-6-chloro-4-methyl-1,1-dioxido-3,4-dihydro-2H-benzo[e][1,2,4]thiadiazin-2-yl)-3-(6-fluoro-2,3-dimethylphenyl) butanoic acid (204 mg, 95.08%).

### Step 8: Synthesis of 5-((1S)-1-(8-(1-(benzyloxy) ethyl)-6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution (2S)-2-(8-(1-(benzyloxy) ethyl)-6-chloro-4-methyl-1,1-dioxido-3,4-dihydro-2H-benzo[e][1,2,4]thiadiazin-2-yl)-3-(6-fluoro-2,3-dimethylphenyl) butanoic acid (255 mg, 0.44 mmol, 1 equiv) in THF was added CDI (215 mg, 1.33 mmol, 3 equiv) in portions at room temperature. The resulting mixture was stirred for 30 min at room temperature. To the above mixture was added hydrazine hydrate (98%) (0.44 mL, 2.7 mmol, 6 equiv) dropwise at 0° C. The resulting mixture was stirred for additional 20 min at 0° C. The reaction was quenched with Water/Ice (2 ml) at room temperature. The resulting mixture was extracted with EtOAc (3×1 mL). The combined organic layers were washed with brine (1×2 mL), dried over anhydrous Na₂SO₄. After filtration, the filtrate was concentrated under reduced pressure. The resulting mixture was diluted with 1,4-dioxane (1 mL). To the above mixture was added CDI (215 mg, 1.33 mmol, 3 equiv) in portions at room temperature. The resulting mixture was stirred for additional 1 h at room temperature. The residue was purified by reversed-phase flash chromatography with the following conditions: column, C18 silica gel; mobile phase, MeCN in Water (10 mmol/L NH₄HCO₃), 10% to 100% gradient in 20 min; detector, UV 254 nm. This resulted in 5-((1S)-1-(8-(1-(benzyloxy) ethyl)-6-chloro-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (220 mg, 80.7%).

### Step 9: Synthesis of 5-((1S,2R)-1-(6-chloro-8-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution of 5-((1S)-1-(8-(1-(benzyloxy) ethyl)-6-chloro-4-methyl-1,1-dioxido-3,4-dihydro-2H-benzo[e][1,2,4] thiadiazin-2-yl)-2-(6-fluoro-2,3-dimethylphenyl)propyl)-1,3,4-oxadiazol-2 (3H)-one (175 mg, 0.28 mmol, 1 equiv) in DCM (1.75 mL) was added boron trichloride (1138 μL, 1.14 mmol, 4 equiv) dropwise at 0° C. under nitrogen atmosphere. The resulting mixture was stirred for 10 min at 0° C. The reaction was quenched with MeOH at room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 10% to 100% gradient in 15 min; detector, UV 254 nm. This resulted 5-((1S)-1-(6-chloro-8-(1-hydroxyethyl)-4-methyl-1,1-dioxido-3,4-dihydro-2H-benzo[e][1,2,4] thiadiazin-2-yl)-2-(6-fluoro-2,3-dimethylphenyl)propyl)-1,3,4-oxadiazol-2 (3H)-one (140 mg, 93.7%).

The crude product (80 mg) was purified by Chiral-Prep-HPLC with the following conditions: Column, XBridge Prep OBD C18 Column, 30*150 mm, 5 μm; mobile phase, 10 mmol, NH₄HCO₃+0.05% NH₃H₂O and ACN (20% ACN up to 50% in 8 min); Detector, uv 254 &220 nm. This resulted in 5-((1S,2R)-1-(6-chloro-8-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (18.2 mg, 23.3%). LCMS: (ES, m/z): M−H=523.0. ¹H NMR (300 MHz, Methanol-d₄) δ 7.07-7.00 (m, 2H), 6.78-6.72 (m, 1H), 6.60-6.58 (t, J=2.6 Hz, 1H), 5.59-5.54 (q, J=6.2 Hz, 1H), 5.46-5.42 (d, J=8.2 Hz, 1H), 5.32-5.27 (d, J=14.5 Hz, 1H), 5.01-4.96 (s, 1H), 4.11-3.86 (m, 1H), 2.89 (s, 3H), 2.41-2.39 (d, J=7.1 Hz, 3H), 2.31-2.29 (d, J=17.4 Hz, 3H), 1.52-1.45 (m, 5H), 1.33-1.31 (d, J=6.9 Hz, 1H).

### Example 69: 5-((1S,2R)-1-(7-chloro-8-(methoxymethyl)-1,1-dioxido-3,4-dihydro-2H-benzo[b][,4,5]oxathiazepin-2-yl)-2-(6-fluoro-2,3-dimethylphenyl)propyl)-1,3,4-oxadiazol-2 (3H)-one

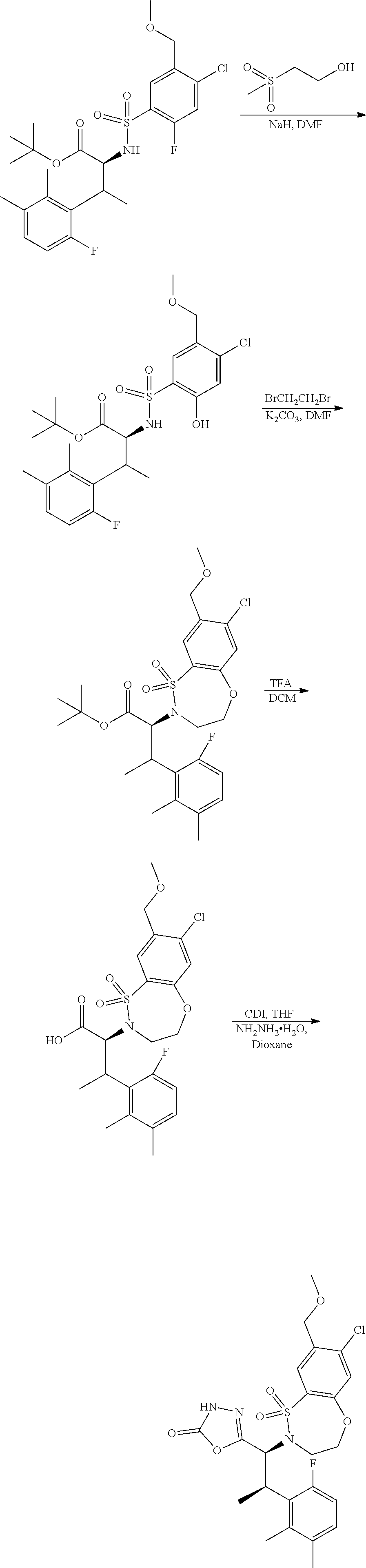

### Step 1: Synthesis of tert-butyl (2S)-2-((4-chloro-2-hydroxy-5-(methoxymethyl) phenyl)sulfonamido)-3-(6-fluoro-2,3-dimethylphenyl)butanoate

To a solution of tert-butyl (2S)-2-[4-chloro-2-fluoro-5-(methoxymethyl) benzenesulfonamido]-3-(6-fluoro-2,3-dimethylphenyl) butanoate (1 g, 1.93 mmol, 1 equiv) in DMF was added sodium hydride (60% in oil, 308.85 mg) at 0° C. The mixture was stirred for 15 min. 2-methanesulfonylethanol (479 mg, 3.86 mmol, 2 equiv) was added and the mixture was allowed to warm to RT and stirred 2 h at room temperature under nitrogen atmosphere. The reaction was quenched with sat. NH₄C1 (aq., 10 ml) at room temperature. The resulting mixture was extracted with EtOAc (3×8 mL). The combined organic layers were washed with brine (3×8 mL), dried over anhydrous Na₂SO₄. The residue was purified by silica gel column chromatography, eluted with PE/EtOAc (10:1) to afford tert-butyl (2S)-2-[4-chloro-2-hydroxy-5-(methoxymethyl) benzenesulfonamido]-3-(6-fluoro-2,3-dimethylphenyl) butanoate (870 mg, 87.3%).

### Step 2: Synthesis of tert-butyl (2S)-2-(7-chloro-8-(methoxymethyl)-1,1-dioxido-3,4-dihydro-2H-benzo[b][1,4,5]oxathiazepin-2-yl)-3-(6-fluoro-2,3-dimethylphenyl)butanoate

Into a 8 mL vial were added tert-butyl (2S)-2-[4-chloro-2-hydroxy-5-(methoxymethyl) benzenesulfonamido]-3-(6-fluoro-2,3-dimethylphenyl) butanoate (400 mg, 0.78 mmol, 1 equiv), DMF (5 mL), dibromoethane (200 μL, 2.33 mmol, 3 equiv) and K₂CO₃ (321 mg, 2.33 mmol, 3 equiv) at room temperature. The resulting mixture was stirred overnight at 65° C. The reaction was quenched with sat.

NH₄C1 (aq., 3 ml) at room temperature. The resulting mixture was extracted with EtOAc (3×2 mL). The combined organic layers were washed with brine (3×2 mL), dried over anhydrous Na₂SO₄. After filtration, the filtrate was concentrated under reduced pressure. The residue was purified by silica gel column chromatography, eluted with PE/EtOAc (5:1) to afford tert-butyl (2S)-2-(7-chloro-8-(methoxymethyl)-1,1-dioxido-3,4-dihydro-2H-benzo[b][1,4,5] oxathiazepin-2-yl)-3-(6-fluoro-2,3-dimethylphenyl)butanoate (308 mg, 73.3%).

### Step 5: Synthesis of (2S)-2-(7-chloro-8-(methoxymethyl)-1,1-dioxido-3,4-dihydro-2H-benzo[b][1,4,5]oxathiazepin-2-yl)-3-(6-fluoro-2,3-dimethylphenyl)butanoic acid

Into a 8 mL sealed tube were added tert-butyl (2S)-2-(7-chloro-8-(methoxymethyl)-1,1-dioxido-3,4-dihydro-2H-benzo[b][1,4,5]oxathiazepin-2-yl)-3-(6-fluoro-2,3-dimethylphenyl)butanoate (301 mg, 0.56 mmol, 1 equiv), DCM (3 mL) and trifluoroacetic acid (3 mL) at room temperature. The resulting mixture was stirred 2 h at room temperature. The resulting mixture was concentrated under vacuum. The crude product was used in the next step directly without further purification.

### Step 4: Synthesis of 5-((1S,2R)-1-(7-chlor-8-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 8 mL sealed tube were added (2S)-2-(7-chloro-8-(methoxymethyl)-1,1-dioxido-3,4-dihydro-2H-benzo[b][1,4,5]oxathiazepin-2-yl)-3-(6-fluoro-2,3-dimethylphenyl)butanoic acid (100 mg, 0.21 mmol, 1 equiv), THF (1 mL) and CDI (150 mg, 0.93 mmol, 4.5 equiv) at room temperature. The resulting mixture was stirred for 40 min at room temperature. To the above mixture was added hydrazine hydrate (90 μL, 1.85 mmol, 9 equiv) dropwise at 0° C. The resulting mixture was stirred for additional 30 min at 0° C. The reaction was quenched with sat. NH₄Cl (aq.) at room temperature. The resulting mixture was extracted with EtOAc (3×1 mL), dried over anhydrous Na₂SO₄. After filtration, the filtrate was concentrated under reduced pressure. To the above mixture was added dioxane (1 mL) and CDI (83 mg, 0.52 mmol, 2.5 equiv) at room temperature. The resulting mixture was stirred for 30 min at room temperature. The reaction was quenched with sat. NH₄Cl (aq.) at room temperature. The resulting mixture was extracted with EtOAc (3×0.5 mL). The combined organic layers were washed with brine (3×0.5 mL), dried over anhydrous Na₂SO₄. After filtration, the filtrate was concentrated under reduced pressure. The crude product was purified by Chiral-Prep-HPLC with the following conditions: Column: XBridge Prep OBD C18 Column, 19*250 mm, 5 μm; Mobile Phase A: 10 mmol NH₄HCO₃+0.05% NH₃H₂O, Mobile Phase B: MeOH-HPLC; Flow rate: 20 mL/min; Gradient: 15% B to 45% B in 8 min, 45% B; Wave Length: 254 nm; RT1 (min): 6.8 to afford 5-((1S,2R)-1-(7-chloro-8-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (9.9 mg, 9.2%). LCMS:(ES, m/z): [M−H]+=524.00 ¹H NMR (300 MHz, DMSO-d₆) δ 12.18 (s, 1H), 7.79 (s, 1H), 7.29 (s, 1H), 7.05-7.00 (dd, J=8.4, 5.8 Hz, 1H), 6.86-6.79 (dd, J=12.2, 8.4 Hz, 1H), 5.43-5.39 (m, 1H), 4.55-4.48 (s, 3H), 3.94-3.78 (m, 4H), 3.37 (s, 3H), 2.27 (s, 3H), 2.16 (s, 3H), 1.32-1.30 (d, J=6.8 Hz, 3H), 1.23 (s, 1H).

### Example 70: 5-((1S,2R)-1-(6-chloro-8-(methoxymethyl)-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

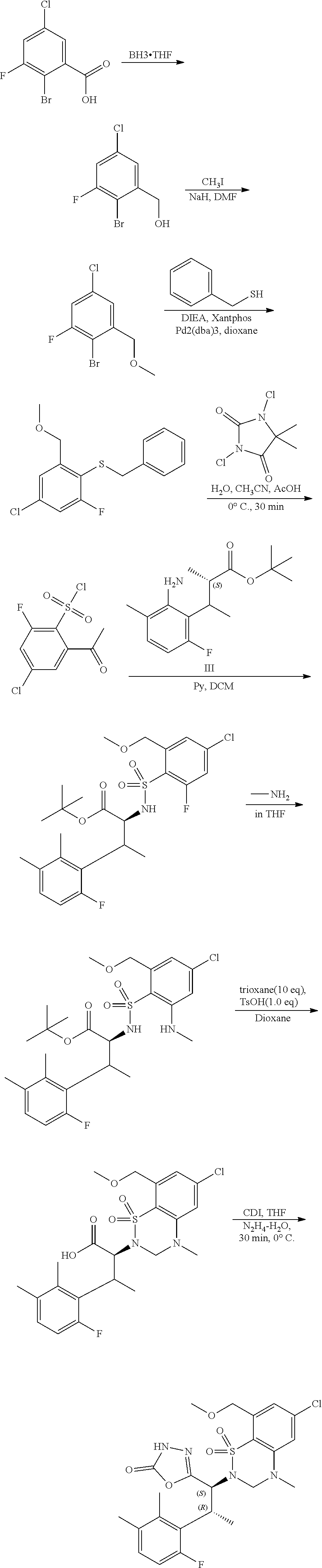

### Step 1: Synthesis of (2-bromo-5-chloro-3-fluorophenyl) methanol

Into a 250 mL round-bottom flask were added 2-bromo-5-chloro-3-fluorobenzoic acid (3.6 g, 14.2 mmol, 1 equiv) and THF (40 mL) at room temperature. To the above mixture was added tetrahydrofuran borane (40 mL, 40 mmol, 2.82 equiv) dropwise at 0° C. The resulting mixture was stirred overnight at room temperature. The reaction was quenched with MeOH (30 ml). The resulting mixture was concentrated under vacuum. The residue was purified by silica gel column chromatography, eluted with PE/EtOAc to afford (2-bromo-5-chloro-3-fluorophenyl) methanol (2.65 g, 77.9%).

### Step 2: Synthesis of 2-bromo-5-chloro-1-fluoro-3-(methoxymethyl)benzene

Into a 100 mL round-bottom flask were added (2-bromo-5-chloro-3-fluorophenyl) methanol (3380 mg, 14.1 mmol, 1 equiv), THF (30 mL). To the mixture was added NaH (842 mg, 35.1 mmol, 2.5 equiv) at 0° C. The resulting mixture was stirred for 30 min at room temperature. To the above mixture was added CH₃I (1050 μL, 16.9 mmol, 1.2 equiv) dropwise at room temperature. The mixture was stirred overnight at room temperature. The resulting mixture was quenched with water (30 ml) and extracted with EtOAc (3×20 mL). The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 2-bromo-5-chloro-1-fluoro-3-(methoxymethyl) benzene (2900 mg, 81.2%).

### Step 3: Synthesis of benzyl(4-chloro-2-fluoro-6-(methoxymethyl)phenyl) sulfane

Into a 100 mL round-bottom flask were added 2-bromo-5-chloro-1-fluoro-3-(methoxymethyl) benzene (3000 mg, 11.8 mmol, 1 equiv), dioxane (30 mL), DIEA (4123 μL, 23.7 mmol, 2 equiv), Xantphos (685 mg, 1.18 mmol, 0.10 equiv), Pd₂(dba)₃ (1084 mg, 1.18 mmol, 0.1 equiv) and benzyl mercaptan (1683 μL, 0.083 mmol, 1.2 equiv) at room temperature. The resulting mixture was stirred overnight at 110° C. under nitrogen atmosphere. The resulting mixture was quenched with H₂O (30 ml) and extracted with EtOAc (50 ml). The combined organic layers were washed with brine (1×30 ml), dried over anhydrous Na₂SO₄. After filtration, the filtrate was concentrated under reduced pressure. The residue was purified by silica gel column chromatography, eluted with PE/EtOAc to afford benzyl(4-chloro-2-fluoro-6-(methoxymethyl) phenyl) sulfane (3000 mg, 85.4%).

### Step 4: Synthesis of 4-chloro-2-fluoro-6-(methoxymethyl)benzenesulfonyl chloride

Into a 100 mL vial were added 2-(benzylsulfanyl)-5-chloro-1-fluoro-3-(methoxymethyl) benzene (3000 mg, 10.1 mmol, 1 equiv), CH₃CN (30 mL), H₂O (1.5 mL) and AcOH (2.1 mL) at room temperature. To the above mixture was added 1,3-dichloro-5,5-dimethylimidazolidine-2,4-dione (3400 mg, 20.2 mmol, 2 equiv) dropwise at 0° C. The resulting mixture was stirred for additional 30 min at 0° C. The resulting mixture was quenched with water (50 ml) and extracted with EtOAc (3×50 mL). The combined organic layers were washed with brine (1×50 ml), dried over anhydrous Na₂SO₄. After filtration, the filtrate was concentrated under reduced pressure. The residue was purified by silica gel column chromatography, eluted with PE/EtOAc (1:1) to afford 4-chloro-2-fluoro-6-(methoxymethyl) benzenesulfonyl chloride (2.5 g, 90.6%).

### Step 5: Synthesis of tert-butyl (2S)-2-((4-chloro-2-fluoro-6-(methoxymethyl) phenyl) sulfonamido)-3-(6-fluoro-2,3-dimethylphenyl) butanoate

Into a 100 mL round-bottom flask were added tert-butyl (2R)-2-amino-3-(6-fluoro-2,3-dimethylphenyl) butanoate (3.01 g, 10.7 mmol, 1 equiv), DCM (30 mL), Pyridine (2.58 mL, 32 mmol, 3 equiv). To the mixture was added 4-chloro-2-fluoro-6-(methoxymethyl) benzenesulfonyl chloride (2.92 g, 10.7 mmol, 1 equiv) at 0° C. The resulting mixture was stirred overnight at room temperature. The resulting mixture was quenched with water (50 ml) and extracted with CH₂Cl₂ (3×100 mL). The combined organic layers were washed with brine (1×50 ml), dried over anhydrous MgSO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in tert-butyl (2S)-2-[4-chloro-2-fluoro-6-(methoxymethyl) benzenesulfonamido]-3-(6-fluoro-2,3-dimethylphenyl) butanoate (3 g, 54.2%).

### Step 6: Synthesis of tert-butyl (2S)-2-((4-chloro-2-(methoxymethyl)-6-(methylamino) phenyl) sulfonamido)-3-(6-fluoro-2,3-dimethylphenyl) butanoate

Into a 100 mL round-bottom flask were added tert-butyl (2S)-2-[4-chloro-2-fluoro-6-(methoxymethyl) benzenesulfonamido]-3-(6-fluoro-2,3-dimethylphenyl) butanoate (1000 mg, 1.93 mmol, 1 equiv), DMSO (10 mL) and Methylamine, 2M in THF (15 mL, 29 mmol, 15 equiv) at room temperature.

The resulting mixture was stirred overnight at 100° C. under nitrogen atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl (2S)-2-[4-chloro-2-(methoxymethyl)-6-(methylamino) benzenesulfonamido]-3-(6-fluoro-2,3-dimethylphenyl) butanoate (1000 mg, 97.9%).

### Step 7: Synthesis of (2S)-2-(6-chloro-8-(methoxymethyl)-4-methyl-1,1-dioxido-3,4-dihydro-2H-benzo[e][1,2,4]thiadiazin-2-yl)-3-(6-fluoro-2,3-dimethylphenyl)butanoic acid

Into a 10 mL sealed tube were added tert-butyl (2S)-2-[4-chloro-2-(methoxymethyl)-6-(methylamino) benzenesulfonamido]-3-(6-fluoro-2,3-dimethylphenyl) butanoate (250 mg, 0.47 mmol, 1 equiv), dioxane (2.5 mL), 1,3,5-trioxane (426 mg, 4.73 mmol, 10 equiv) and TsOH (40.7 mg, 0.24 mmol, 0.5 equiv) at room temperature. The resulting mixture was stirred for overnight at 100° C. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in of (2S)-2-(6-chloro-8-(methoxymethyl)-4-methyl-1,1-dioxido-3,4-dihydro-2H-benzo[e][1,2,4]thiadiazin-2-yl)-3-(6-fluoro-2,3-dimethylphenyl)butanoic acid (180 mg, 78.6%).

### Step 8: Synthesis of 5-((1S,2R)-1-(6-chloro-8-(methoxymethyl)-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

Into a 8 mL vial were added (2S)-2-(6-chloro-8-(methoxymethyl)-4-methyl-1,1-dioxido-3,4-dihydro-2H-benzo[e][1,2,4]thiadiazin-2-yl)-3-(6-fluoro-2,3-dimethylphenyl)butanoic acid (50 mg, 0.10 mmol, 1 equiv), THF (1 mL) and CDI (134 mg, 0.82 mmol, 8 equiv) at room temperature. The resulting mixture was stirred for 50 min at room temperature. To the above mixture was added hydrazine hydrate (25 μL, 0.52 mmol, 5 equiv) dropwise at 0° C. The resulting mixture was stirred for additional 30 min at 0° C.

The resulting mixture was quenched with water (20 ml) and extracted with EtOAc (3×20 mL). The combined organic layers were washed with brine (1×20 ml), dried over anhydrous Na₂SO₄. After filtration, the filtrate was concentrated under reduced pressure. To the above mixture was added dioxane (1 mL) and CDI (134 mg, 0.82 mmol, 8 equiv) dropwise at room temperature. The resulting mixture was stirred overnight at room temperature. The residue was purified by reversed-phase flash chromatography with the following conditions: column, C18 silica gel; mobile phase, MeCN in Water (0.1% FA), 10% to 50% gradient in 10 min; detector, UV 254 nm. The crude product was purified by Chiral-Prep-HPLC with the following conditions: Column, XBridge Prep OBD C18 Column, 30*150 mm, 5 μm; mobile phase, Water (10 mmol/L NH₄HCO₃+0.1% NH₃·H₂O) and ACN (25% ACN up to 45% in 8 min); Detector, uv 254 &220 nm. This resulted in 5-((1S,2R)-1-(6-chloro-8-(methoxymethyl)-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (12.5 mg, 22.4%). LCMS: (ES, m/z): M−H=523.05. ¹H NMR (300 MHz, Methanol-d₄) δ 7.12-6.70 (m, 3H), 6.63 (dd, J=3.9, 2.0 Hz, 1H), 5.52 (dd, J=11.8, 1.6 Hz, 1H), 5.44-5.24 (m, 1H), 4.99 (d, J=14.6 Hz, 1H), 4.80 (d, J=6.9 Hz, 1H), 4.76-4.65 (m, 1H), 4.62 (d, J=5.8 Hz, 1H), 3.99-3.82 (m, 1H), 3.46 (s, 2H), 3.30 (s, 1H), 2.88 (s, 3H), 2.37 (d, J=9.2 Hz, 3H), 2.26 (d, J=15.5 Hz, 3H), 1.45 (dd, J=6.9, 1.1 Hz, 2H), 1.29 (d, J=6.8 Hz, 1H).

### Example 71: 5-((1S,2R)-1-(7-chloro-9-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

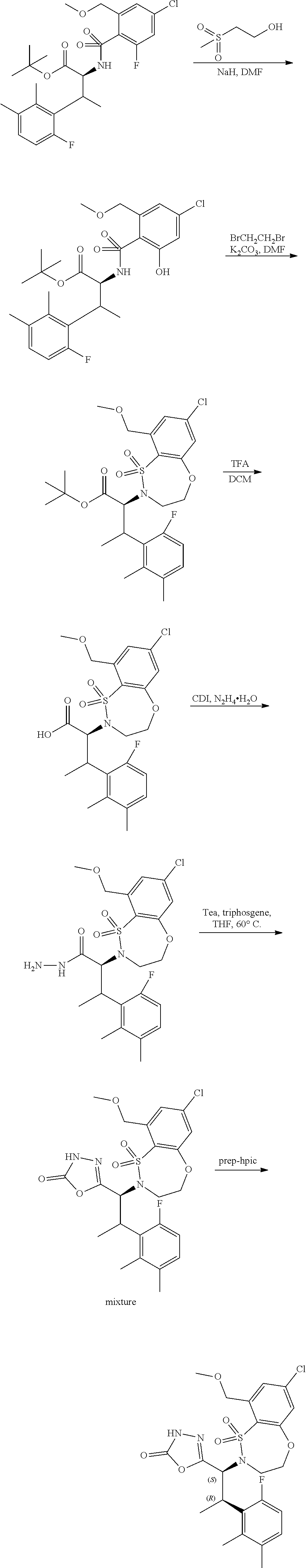

### Step 1: Synthesis of tert-butyl (2S)-2-((4-chloro-2-hydroxy-6-(methoxymethyl) phenyl) sulfonamido)-3-(6-fluoro-2,3-dimethylphenyl) butanoate

Into a 100 mL 3-necked round-bottom flask were added tert-butyl (2S)-2-[4-chloro-2-fluoro-6-(methoxymethyl) benzenesulfonamido]-3-(6-fluoro-2,3-dimethylphenyl) butanoate (960 mg, 1.9 mmol, 1 equiv) and 2-methanesulfonylethanol (0.46 g, 3.7 mmol, 2 equiv) at room temperature. To the above mixture was added NaH (0.37 g, 9.27 mmol, 5 equiv, 60%) in portions at 0° C. The resulting mixture was stirred overnight at room temperature. The resulting mixture was quenched with water (50 ml) and extracted with EtOAc (2×50 mL). The combined organic layers were washed with brine (1×50 mL), dried over anhydrous Na₂SO₄. After filtration, the filtrate was concentrated under reduced pressure. This resulted in tert-butyl (2S)-2-[4-chloro-2-hydroxy-6-(methoxymethyl) benzenesulfonamido]-3-(6-fluoro-2,3-dimethylphenyl) butanoate (330 mg, 34.5%).

### Step 2: Synthesis of tert-butyl (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl) butanoate

Into a 20 mL sealed tube were added tert-butyl (2S)-2-[4-chloro-2-hydroxy-6-(methoxymethyl) benzenesulfonamido]-3-(6-fluoro-2,3-dimethylphenyl) butanoate (20 mg, 0.039 mmol, 1 equiv), DMF (4.80 mL), dibromoethane (60 μL, 0.7 mmol, 1.5 equiv) and K₂CO₃ (129 mg, 0.93 mmol, 2 equiv) at room temperature. The resulting mixture was stirred overnight at 60° C. under air atmosphere. The resulting mixture was quenched with water (50 ml) and extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in tert-butyl (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl) butanoate (160 mg, 63.5%).

### Step 3: Synthesis of (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl) butanoic acid

Into a 100 mL round-bottom flask were added tert-butyl (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl) butanoate (268 mg, 0.49 mmol, 1 equiv), DCM (1 mL) and TFA (3 mL) at room temperature. The resulting mixture was stirred for 1 h at room temperature under air atmosphere. The resulting mixture was concentrated under reduced pressure. The crude product was used in the next step directly without further purification.

### Step 4: Synthesis of (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl)butanehydrazide

Into a 50 mL round-bottom flask were added (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl) butanoic acid (268 mg, 0.55 mmol, 1 equiv), THF, CDI (268.28 mg, 1.65 mmol, 3 equiv) and NH₂NH₂·H₂O (138 mg, 2.76 mmol, 5 equiv) at room temperature. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2S)-2-(7-chloro-9-(methoxymethyl)-1,1-dioxido-3,4-dihydro-2H-benzo[b][1,4,5] oxathiazepin-2-yl)-3-(6-fluoro-2,3-dimethylphenyl)butanehydrazide (190 mg, 68.9/6).

### Step 5: Synthesis of 5-((1S,2R)-1-(7-chloro-9-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

Into a 10 mL sealed tube were added (2S)-2-(7-chloro-9-(methoxymethyl)-1,1-dioxido-3,4-dihydro-2H-benzo[b][1,4,5]oxathiazepin-2-yl)-3-(6-fluoro-2,3-dimethylphenyl)butanehydrazide (130 mg, 0.260 mmol, 1 equiv), THF (3 mL), DIEA (113.23 μL, 0.650 mmol, 2.5 equiv) and triphosgene (38.6 mg, 0.13 mmol, 0.5 equiv) at room temperature. The resulting mixture was stirred for 1 h at 80° C. under air atmosphere. The residue was purified by reversed-phase flash chromatography with the following conditions: column, C18 silica gel; mobile phase, MeCN in Water (0.1% NH₃·H₂O), 60% to 70% gradient in 10 min; detector, UV 254 nm. This resulted in 5-((1S)-1-(7-chloro-9-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (103 mg, 75.3%). The residue was purified by reversed-phase flash chromatography with the following conditions: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 26% B to 52% B in 9 min, 52% B; Wave Length: 254 nm; RT1 (min): 7. This resulted in 5-((1S,2R)-1-(7-chloro-9-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (17.7 mg, 24.6%). LCMS:(ES, m/z):[M−H]⁺=524.05. ¹H NMR (300 MHz, DMSO-d₆) δ 7.67-7.47 (m, OH), 7.37 (d, J=2.2 Hz, 1H), 7.14-6.98 (m, 2H), 6.83 (dd, J=12.2, 8.4 Hz, 1H), 5.42 (dd, J=11.7, 1.9 Hz, 1H), 4.87-4.68 (m, 2H), 4.50-4.36 (m, 1H), 4.36-4.25 (m, 1H), 3.78 (d, J=3.1 Hz, 3H), 3.17 (s, 2H), 2.92 (d, J=15.9 Hz, 3H), 2.24 (d, J=34.7 Hz, 6H), 1.34 (d, J=6.8 Hz, 3H).

### Example 72:5-((1S,2R)-1-(6-chloro-8-(hydroxymethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

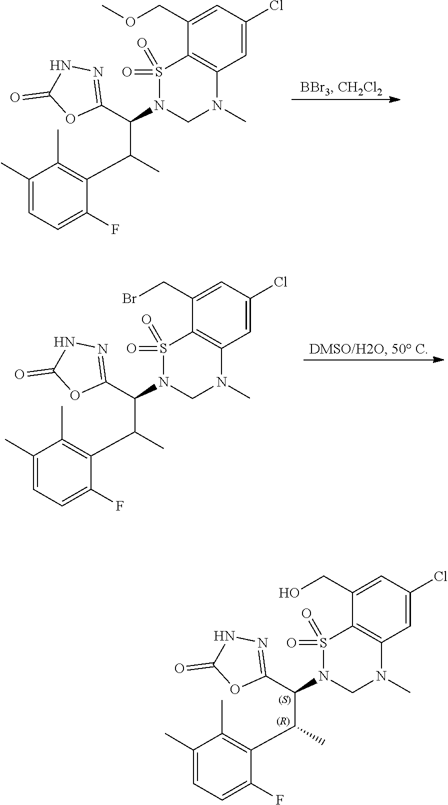

### Step 1: Synthesis of 5-((1S)-1-(8-(bromomethyl)-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

Into a 8 mL vial were added 5-((1S)-1-(6-chloro-8-(methoxymethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (80 mg, 0.15 mmol, 1 equiv), DCM (2 mL) and BBr₃ (380 μL, 0.76 mmol, 5 equiv) at room temperature. The mixture was stirred for 30 min at room temperature. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-((1S)-1-(8-(bromomethyl)-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (60 mg, 68.6%).

### Step 2: Synthesis of 5-((1S,2R)-1-(6-chloro-8-(hydroxymethyl)-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

Into a 8 mL vial were added 5-((1S)-1-(8-(bromomethyl)-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one(50 mg, 0.087 mmol, 1 equiv), DMSO (0.1 mL) and H₂O (1 mL) at room temperature. The resulting mixture was stirred for overnight at 50° C. The residue was purified by reversed-phase flash chromatography with the following conditions: column, C18 silica gel; mobile phase, MeCN in Water (0.1% FA), 10% to 50% gradient in 10 min; detector, UV 254 nm. The crude product (was purified by Chiral-Prep-HPLC with the following conditions: Column, XBridge Prep OBD C18 Column, 30*150 mm, 5 μm; mobile phase, Water (10 mmol/L NH₄HCO₃+0.1% NH₃·H₂O) and ACN (25% ACN up to 45% in 8 min); Detector, uv 254&220 nm. This resulted in 5-((1S,2R)-1-(6-chloro-8-(hydroxymethyl)-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (13 mg, 28.91%). LCMS:(ES, m/z): M−H=509.05. ¹H NMR (300 MHz, Methanol-d₄) δ 7.07 (d, J=2.0 Hz, 1H), 7.00 (dd, J=8.4, 5.7 Hz, 1H), 6.73 (dd, J=12.1, 8.4 Hz, 1H), 6.62 (d, J=2.0 Hz, 1H), 5.53 (dd, J=11.9, 1.7 Hz, 1H), 5.28 (d, J=14.6 Hz, 1H), 5.00 (d, J=2.2 Hz, 1H), 4.95 (d, J=1.5 Hz, 1H), 4.61 (s, 1H), 3.90 (dtd, J=12.5, 7.1, 5.5 Hz, 1H), 2.87 (s, 3H), 2.39 (s, 3H), 2.23 (s, 3H), 1.44 (dd, J=6.9, 1.1 Hz, 3H).

### Example 73:5-((1S,2R)-1-(6-chloro-4-(2-hydroxyethyl)-1,1-dioxido-3,4-dihydro-2H-pyrido[2,3-e][1,2,4]thiadiazin-2-yl)-2-(6-fluoro-2,3-dimethylphenyl) propyl)-1,3,4-oxadiazol-2 (3H)-one

The title compound was prepared as ex-50, using 2-(Benzyloxy)-1-ethanamine and (2S,3R)-2-(6-chloro-2-fluoropyridine-3-sulfonamido)-3-(6-fluoro-2,3-dimethylphenyl)butanoic acid in place of (2S,3R)-2-(4-chloro-2-fluorobenzene sulfonamido)-3-(6-fluoro-2,3-dimethylphenyl) butanoic acid to obtain the final product 5-((1S,2R)-1-(6-chloro-4-(2-hydroxyethyl)-1,1-dioxido-3,4-dihydro-2H-pyrido[2,3-e][1,2,4]thiadiazin-2-yl)-2-(6-fluoro-2,3-dimethylphenyl) propyl)-1,3,4-oxadiazol-2 (3H)-one (0.011 g, 0.021 mmol, yield 47%). LCMS:(ES, m/z): M+H+=512.33. ¹H NMR (300 MHz, DMSO-d₆) δ 12.16 (s, 1H1), 8.02 (d, J=8.0 Hz, 1H), 7.06-6.98 (m, 1H), 6.86-6.77 (m, 2H), 5.46-5.29 (m, 2H), 5.26 (d, J=11.7 Hz, 1H), 4.95 (t, J=5.1 Hz, 1H), 4.06-3.87 (m, 2H), 3.61-3.51 (m, 2H), 3.08 (dt, J=13.5, 6.7 Hz, 1H), 2.33 (s, 3H), 2.17 (s, 3H), 1.33 (d, J=6.8 Hz, 3H).

### Example 74: 5-((1S,2R)-1-(6-chloro-4-((R)-1-methylpyrrolidin-3-yl)-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

The title compound was synthesized following the procedure described in example 30 using (3R)-1-methylpyrrolidin-3-amine instead of 2-methoxypropylamine to obtain 5-((1S,2R)-1-(6-chloro-4-((R)-1-methylpyrrolidin-3-yl)-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (0.018 g, yield 33%). LCMS (ES, m/z): M+H+=550.20. ¹H NMR (400 MHz, Methanol-d₄) 7.57 (d, J=8.4 Hz, 1H), 7.04 (d, J=1.8 Hz, 1H), 6.99 (dd, J=8.4, 5.7 Hz, 1H), 6.81 (dd, J=8.4, 1.8 Hz, 1H), 6.72 (dd, J=12.1, 8.4 Hz, 1H), 5.46 (dd, J=11.8, 1.6 Hz, 1H), 5.28 (d, J=14.5 Hz, 1H), 5.22 (d, J=14.5 Hz, 1H), 4.47-4.39 (m, 1H), 3.92 (dq, J=13.4, 6.9 Hz, 1H), 3.20 (dd, J=11.2, 3.6 Hz, 1H), 3.03-2.96 (m, 1H), 2.58 (dd, J=11.1, 7.8 Hz, 1H), 2.45 (s, 3H), 2.39 (s, 3H), 2.36-2.27 (m, 2H), 2.23 (s, 3H), 1.91-1.79 (m, 1H), 1.45 (dd, J=6.9, 1.1 Hz, 3H).

### Example 75: 5-((1S,2R)-1-(8-acetyl-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

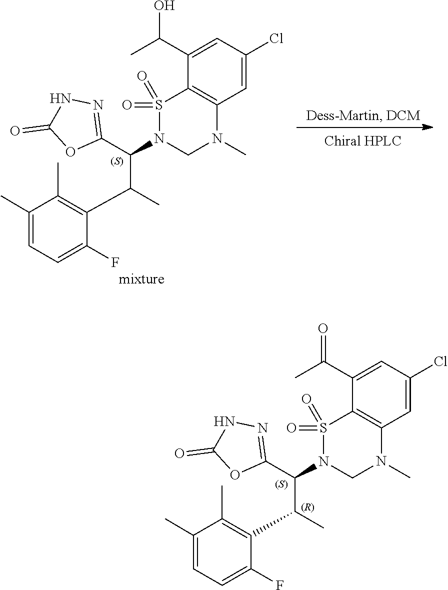

To a stirred solution of 5-((1S)-1-(6-chloro-8-(1-hydroxyethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (240 mg, 0.457 mmol, 1 equiv) (see Example 68) in DCM (4.80 mL) was added Dess-Martin (380 mg, 0.914 mmol, 2 equiv) in portions at 0° C. The resulting mixture was stirred for 30 min at room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 5-((1S)-1-(8-acetyl-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (200 mg, 83.65%).

The crude product (200 mg) was purified by Chiral-Prep-HPLC with the following conditions: Column, YMC-Actus Triart C18 ExRS, 20*250 mm, 5 μm; mobile phase, 10 mmol NH₄HCO₃+0.05% NH₃H₂O and ACN (25% ACN up to 55% in 8 min); Detector, uv 254&220 nm. This resulted in 5-((1S,2R)-1-(8-acetyl-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (62.4 mg, 30.3%). LCMS: (ES, m/z): M−H=521.1. ¹H NMR (300 MHz, Methanol-d₄) δ 7.07-7.03 (dd, J=8.4, 5.8 Hz, 1H), 6.83-6.75 (m, 3H), 5.29-5.25 (d, J=11.5 Hz, 1H), 5.04-4.99 (d, J=14.4 Hz, 1H), 4.79-4.74 (d, J=14.4 Hz, 1H), 4.02-3.96 (dq, J=13.4, 6.9 Hz, 1H), 2.94 (s, 3H), 2.56 (s, 3H), 2.34 (s, 3H), 2.27 (s, 3H), 1.35-1.22 (m, 3H).

### Example 76: 5-((1S,2R)-1 (8-(bromomethyl)-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

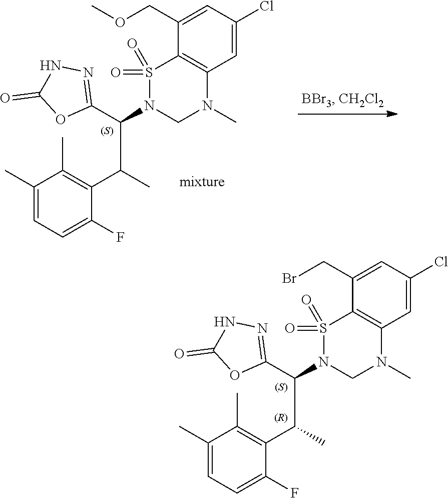

Into a 8 mL vial were added 5-((1S)-1-(6-chloro-8-(methoxymethyl)-4-methyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (50 mg, 0.095 mmol, 1 equiv), DCM (2 mL) and BBr₃ (476 μL, 0.48 mmol, 5 equiv) at room temperature. The resulting mixture was stirred for 30 min at room temperature. The resulting mixture was concentrated under vacuum. The residue was purified by reversed-phase flash chromatography with the following conditions: column, C18 silica gel; mobile phase, MeCN in Water (0.1% FA), 10% to 50% gradient in 10 min; detector, UV 254 nm. The crude product was purified by Chiral-Prep-HPLC with the following conditions: Column, Xselect CSH C18 OBD Column 30*150 mm 5 μm; mobile phase, Water (10 mmol/L NH₄HCO₃+0.1% NH₃·H₂O) and ACN (10% ACN up to 40% in 8 min); Detector, uv 254 &220 nm. This resulted in 5-((1S,2R)-1-(8-(bromomethyl)-6-chloro-4-methyl-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (11.0 mg, 18.6%). LCMS: (ES, m/z): M+H=575.00. ¹H NMR (300 MHz, DMSO-d₆) δ 12.11 (s, 1H), 7.08-7.01 (m, 1H), 6.99 (d, J=1.9 Hz, 1H), 6.83 (dd, J=12.3, 8.4 Hz, 1H), 6.75 (d, J=2.0 Hz, 1H), 5.37 (d, J=11.7 Hz, 1H), 5.30-5.15 (m, 2H), 5.14-5.06 (m, 1H), 4.81 (d, J=10.3 Hz, 1H), 3.94 (dd, J=11.9, 6.7 Hz, 1H), 2.83 (s, 3H), 2.34 (s, 3H), 2.19 (s, 3H), 1.35 (d, J=6.7 Hz, 3H).

### Example 77: 5-((1S,2R)-1-(7-chloro-8-(hydroxymethyl)-1,1-dioxido-3,4-dihydro-2H-benzo[b][1,4,5]oxathiazepin-2-yl)-2-(6-fluoro-2,3-dimethylphenyl) propyl)-1,3,4-oxadiazol-2 (3H)-one

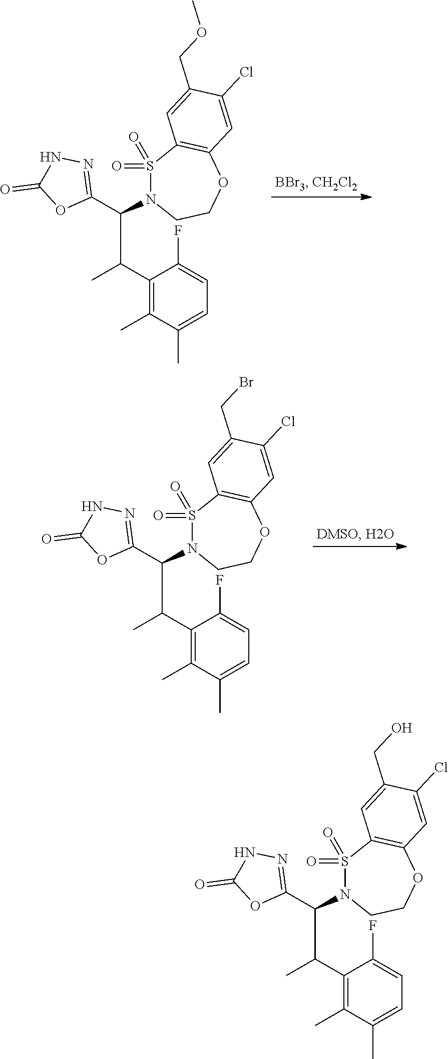 [SMILES: COCc1cc2c(cc1Cl)OCCN([C@H](c1n[nH]c(=O)o1)C(C)c1c(F)ccc(C)c1C)S2(=O)=O ; Cc1ccc(F)c(C(C)[C@@H](c2n[nH]c(=O)o2)N2CCOc3cc(Cl)c(CBr)cc3S2(=O)=O)c1C ; Cc1ccc(F)c(C(C)[C@@H](c2n[nH]c(=O)o2)N2CCOc3cc(Cl)c(CO)cc3S2(=O)=O)c1C]

### Step 1: Synthesis of 5-((1S)-1-(8-(bromomethyl)-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one

To a stirred solution of 5-((1S)-1-(7-chloro-8-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (150 mg, 0.29 mmol, 1 equiv) in DCM was added BBr3 (135 μL, 1.43 mmol, 5 equiv) dropwise at 0° C. under nitrogen atmosphere. The resulting mixture was stirred for 10 min at 0° C. under nitrogen atmosphere. The reaction was quenched with sat. Na2CO3 (aq., 2 ml) at 0° C. The resulting mixture was extracted with CH₂Cl₂ (3×1 mL). The combined organic layers were washed with brine (3×1 mL), dried over anhydrous MgSO₄. After filtration, the filtrate was concentrated under reduced pressure. The crude product mixture was used in the next step directly without further purification.

### Step 2: Synthesis of 5-((1S)-1-(8-(bromomethyl)-7-chloro-1,1-dioxido-3,4-dihydro-2H-benzo[b][1,4,5]oxathiazepin-2-yl)-2-(6-fluoro-2,3-dimethylphenyl) propyl)-1,3,4-oxadiazol-2 (3H)-one

Into a 8 mL sealed tube were added of 5-((1S)-1-(8-(bromomethyl)-7-chloro-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (180 mg, 0.31 mmol, 1 equiv), DMSO (2 mL) and H₂O (1 mL) at room temperature. The resulting mixture was stirred for 1 h at 50° C. The residue was purified by reversed-phase flash chromatography with the following conditions: Column: XselectCSH CIS OBDColumn 30*150 mm 5 μm, n; Mobile Phase A: Water (0.1% FA), Mobile Phase B: ACN; Flow rate: 60 mL/min; Gradient: 15% B to 45% B in 8 min, 45% B; Wave Length: 254 nm; RT1 (min): 7 to afford 7-chloro-2-[(1S)-2-(6-fluoro-2,3-dimethylphenyl)-1-(5-oxo-4H-1,3,4-oxadiazol-2-yl)propyl]-8-(hydroxymethyl)-3,4-dihydro-5,1lambda6,2-benzoxathiazepine-1,1-dione (21.2 mg, 13.2%). LCMS−:(ES, m/z): [M+H]+=510.00. ¹H NMR (400 MHz, DMSO-d₆) δ 12.31-12.10 (s, 1H), 8.27 (s, 1H), 7.87 (s, 1H), 7.24 (s, 1H), 7.04-7.00 (dd, J=8.4, 5.8 Hz, 1H), 6.85-6.80 (dd, J=12.3, 8.4 Hz, 1H), 5.68 (s, 1H), 5.43-5.40 (d, J=11.1 Hz, 1H), 4.53-4.48 (m, 3H), 3.90-3.77 (m, 3H), 2.27 (s, 3H), 2.16 (s, 3H), 1.32-1.30 (d, J=6.8 Hz, 3H).

### Example 78: 5-((1S,2R)-1-(7-chloro-9-(hydroxymethyl)-1,1-dioxido-3,4-dihydro-2H-benzo[b][1,4,5]oxathiazepin-2-yl)-2-(6-fluoro-2,3-dimethylphenyl) propyl)-1,3,4-oxadiazol-2 (3H)-one

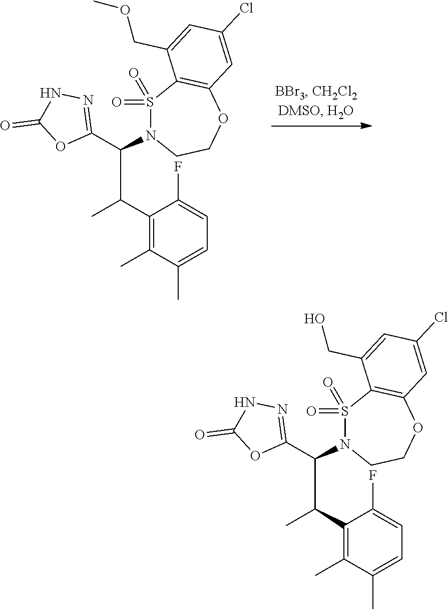 [SMILES: COCc1cc(Cl)cc2c1S(=O)(=O)N([C@H](c1n[nH]c(=O)o1)C(C)c1c(F)ccc(C)c1C)CCO2 ; Cc1ccc(F)c([C@@H](C)[C@@H](c2n[nH]c(=O)o2)N2CCOc3cc(Cl)cc(CO)c3S2(=O)=O)c1C]

Into a 25 mL round-bottom flask were added 5-((1S)-1-(7-chloro-9-(methoxymethyl)-1,1-dioxido-3,4-dihydro-2H-benzo[b][1,4,5]oxathiazepin-2-yl)-2-(6-fluoro-2,3-dimethylphenyl)propyl)-1,3,4-oxadiazol-2 (3H)-one (140 mg, 0.27 mmol, 1 equiv) and DCM (3 mL) at room temperature. To the above mixture was added BBr3 (95 μL, 0.095 mmol, 5 equiv) dropwise at 0° C. The mixture was stirred for additional 10 min at 0° C. The resulting mixture was quenched with H₂O (30 ml) and extracted with EtOAc (2×30 mL), dried over anhydrous Na₂SO₄. After filtration, the filtrate was concentrated under reduced pressure. Into a 20 mL sealed tube were added the above crude product, DMSO (3 mL) and H₂O (1 mL) at room temperature. The resulting mixture was stirred overnight at 50° C. under air atmosphere. The resulting mixture was quenched with water (30 ml) and extracted with EtOAc (3×30 mL). The combined organic layers were washed with brine (1×50 mL), dried over anhydrous Na₂SO₄. After filtration, the filtrate was concentrated under reduced pressure.

The residue was purified by reversed-phase flash chromatography with the following conditions: Column: XBridge Prep Phenyl OBD Column, 19*250 mm, 5 μm; Mobile Phase A: 10 mmol NH₄HCO₃+0.05% NH₃H₂O, Mobile Phase B: MeOH-HPLC; Flow rate: 60 mL/min; Gradient: 18% B to 48% B in 8 min, 48% B; Wavelength: 254 nm; RT1 (min): 8.7. This resulted in 5-((1S,2R)-1-(7-chloro-9-(hydroxymethyl)-1,1-dioxido-3,4-dihydro-2H-benzo[b][1,4,5]oxathiazepin-2-yl)-2-(6-fluoro-2,3-dimethylphenyl) propyl)-1,3,4-oxadiazol-2 (3H)-one (31.5 mg). ¹H NMR (300 MHz, Methanol-d₄) δ 7.55 (d, J=2.6 Hz, 1H), 7.11-6.99 (m, 1H), 6.96 (d, J=2.8 Hz, 1H), 6.74 (ddd, J=11.4, 8.3, 2.4 Hz, 1H), 5.60 (d, J=11.8 Hz, 1H), 5.12-5.04 (m, 1H), 5.01 (d, J=2.5 Hz, 1H), 4.65-4.44 (m, 1H), 4.39 (d, J=6.1 Hz, 1H), 4.06-3.90 (m, 1H), 3.90-3.73 (m, 2H), 2.38 (d, J=2.5 Hz, 3H), 2.24 (d, J=2.4 Hz, 3H), 1.47 (dd, J=6.9, 2.4 Hz, 3H). LCMS: (ES, m/z): [M+H]+=510.05.

### Example 79: 5-((1S,2R)-1-(6-chloro-4-(3-hydroxypropyl)-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one

The title compound was prepared according to the procedure used in example 50 using 3-(Benzyloxy)propan-1-amine instead of 2-benzyloxyethylamine to obtain the final 5-((1S,2R)-1-(6-chloro-4-(3-hydroxypropyl)-1,1-dioxido-3,4-dihydro-2H-benzo[e][1,2,4]thiadiazin-2-yl)-2-(6-fluoro-2,3-dimethylphenyl) propyl)-1,3,4-oxadiazol-2 (3H)-one (0.0071 g, yield 21%). LCMS: (ES, m/z): [M−H]−=523.15. ¹H NMR (300 MHz, Methanol-d₄) δ 7.57 (d, J=8.3 Hz, 1H), 6.99 (dd, J=8.3, 5.9 Hz, 1H), 6.83-6.75 (m, 2H), 6.70 (dd, J=10.0, 2.6 Hz, 1H), 5.44 (d, J=6.0 Hz, 1H), 5.40 (d, J=8.8 Hz, 1H), 5.08 (d, J=14.6 Hz, 1H), 3.91 (dd, J=12.2, 6.7 Hz, 1H), 3.71-3.48 (m, 3H), 3.28-3.21 (m, 1H), 2.38 (s, 3H), 2.22 (s, 3H), 1.80 (p, J=6.6 Hz, 2H), 1.45 (d, J=6.9 Hz, 3H).

### Example 80: 5-((1S,2R)-1-(6-chloro-1,1-dioxido-4-(2-(pyrrolidin-1-yl)ethyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for example 30 using N-(2-aminoethyl)pyrrolidine instead of 2-methoxypropylamine to obtain 5-((1S,2R)-1-(6-chloro-1,1-dioxido-4-(2-(pyrrolidin-1-yl)ethyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.056 g, yield 51%). LCMS: (ES, m/z): [M+H]+=564.25. ¹H NMR (400 MHz, Methanol-d₄) δ 7.57 (d, J=8.3 Hz, 1H), 7.02-6.93 (m, 1H), 6.81-6.74 (m, 2H), 6.74-6.66 (m, 1H), 5.48-5.39 (m, 2H), 5.12 (d, J=14.6 Hz, 1H), 3.91 (dq, J=13.5, 7.6, 6.9 Hz, 1H), 3.60-3.50 (m, 1H), 3.42-3.34 (m, 1H), 2.79-2.61 (m, 6H), 2.39 (s, 3H), 2.22 (s, 3H), 1.85 (s, 4H), 1.45 (d, J=6.9 Hz, 3H).

### Example 81: 5-[(1S,2R)-1-[6-chloro-4-(2,2-difluoroethyl)-1,1-dioxo-3H-pyrido[2,3-e][1,2,4]thiadiazin-2-yl]-2-(6-fluoro-2,3-dimethyl-phenyl)propyl]-3H-1,3,4-oxadiazol-2-one

The title compound was synthesized following the procedure described in Example 30B using 2,2-difluoroethylamine instead of 2-methoxypropylamine and (2S,3R)-2-(6-chloro-2-fluoropyridine-3-sulfonamido)-3-(6-fluoro-2,3-dimethylphenyl)butanoic acid in place of (2S,3R)-2-(4-chloro-2-fluorobenzenesulfonamido)-3-(6-fluoro-2,3-dimethylphenyl) butanoic acid to obtain 5-[(1S,2R)-1-[6-chloro-4-(2,2-difluoroethyl)-1,1-dioxo-3H-pyrido[2,3-e][1,2,4]thiadiazin-2-yl]-2-(6-fluoro-2,3-dimethyl-phenyl)propyl]-3H-1,3,4-oxadiazol-2-one (0.022 g, yield 25%). LCMS: (ES, m/z): [M+H]+=530.13. ¹H NMR (300 MHz, Methanol-d₄) δ 7.97 (dd, J=8.1, 1.5 Hz, 1H), 7.06-6.95 (m, 1H), 6.90 (d, J=8.1 Hz, 1H), 6.73 (dd, J=11.9, 8.7 Hz, 1H), 6.28-5.84 (m, 1H), 5.49 (d, J=15.3 Hz, 1H), 5.43 (d, J=12.8 Hz, 1H), 5.32 (d, J=14.9 Hz, 1H), 4.49-4.29 (m, 1H), 4.06-3.89 (m, 1H), 3.52-3.38 (m, 1H), 2.38 (s, 3H), 2.22 (s, 3H), 1.45 (d, J=6.8 Hz, 3H).

### Example 82: 5-[(1S,2R)-1-[6-chloro-4-(3-hydroxypropyl)-1,1-dioxo-3H-pyrido[2,3-e][1,2,4]thiadiazin-2-yl]-2-(6-fluoro-2,3-dimethyl-phenyl) propyl]-3H-1,3,4-oxadiazol-2-one

The title compound was prepared as ex-73, using 3-(Benzyloxy)-1-propylamine instead of 2-(Benzyloxy)-1-ethylamine and (2S,3R)-2-(6-chloro-2-fluoropyridine-3-sulfonamido)-3-(6-fluoro-2,3-dimethylphenyl)butanoic acid in place of (2S,3R)-2-(4-chloro-2-fluorobenzene sulfonamido)-3-(6-fluoro-2,3-dimethylphenyl) butanoic acid to obtain the final product 5-[(1S,2R)-1-[6-chloro-4-(3-hydroxypropyl)-1,1-dioxo-3H-pyrido[2,3-e][1,2,4]thiadiazin-2-yl]-2-(6-fluoro-2,3-dimethyl-phenyl) propyl]-3H-1,3,4-oxadiazol-2-one (0.028 g, yield 85%). LCMS: (ES, m/z): [M+H]+=526.16. ¹H NMR (400 MHz, Methanol-d₄) δ 7.89 (d, J=8.0 Hz, 1H), 7.00 (dd, J=8.5, 5.8 Hz, 1H), 6.77 (d, J=8.0 Hz, 1H), 6.72 (dd, J=12.2, 8.4 Hz, 1H), 5.48-5.38 (m, 2H), 5.19 (d, J=14.9 Hz, 1H), 4.09-3.99 (m, 1H), 3.93 (dd, J=12.1, 6.6 Hz, 1H), 3.61 (dq, J=11.9, 6.1 Hz, 3H), 3.20 (dt, J=14.2, 7.2 Hz, 1H), 2.39 (s, 3H), 2.23 (s, 3H), 1.84 (dq, J=13.5, 6.7 Hz, 2H), 1.46 (dd, J=6.9, 1.1 Hz, 3H).

### Example 83: 5-((1S,2R)-1-(6-chloro-4-(oxetan-3-ylmethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described in Example 30 using oxetan-3-ylmethanamine instead of 2-methoxypropylamine to obtain: 5-((1S,2R)-1-(6-chloro-4-(oxetan-3-ylmethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.019 g, yield 29%). LCMS: (ES, m/z): [M−H]−=535.13. ¹H NMR (400 MHz, Methanol-d₄) δ 7.57 (d, J=8.3 Hz, 1H), 6.99 (dd, J=8.4, 5.8 Hz, 1H), 6.80 (m, 1H), 6.72 (dd, J=12.1, 8.4 Hz, 1H), 5.49-5.19 (q, J=14.9 Hz 2H), 5.34 (s, 1H), 4.53 (dt, J=25.8, 6.2 Hz, 2H), 3.95 (dd, J=13.7, 7.0 Hz, 1H), 3.79 (dd, J=15.3, 5.9 Hz, 1H), 3.57 (dd, J=15.4, 8.5, 1H) 3.38 (dd, J=14.0, 7.0 Hz, 1H), 2.40 (s, 3H), 2.22 (s, 3H), 1.46 (dd, J=6.8, 1.1 Hz, 3H).

### Example 84: 5-((1S,2R)-1-(6-chloro-4-(2-morpholinoethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described in example 30 using 2-morpholinoethan-1-amine to obtain 5-((1S,2R)-1-(6-chloro-4-(2-morpholinoethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one. LCMS: (ES, m/z): [M−H]−=578.3. ¹H NMR (400 MHz, Methanol-d₄) δ 7.58 (d, J=8.2 Hz, 1H), 6.99 (dd, J=8.4, 5.8 Hz, 1H), 6.79 (m, 1H), 6.72 (dd, J=12.1, 8.4 Hz, 1H), 5.53-5.11 (q, J=14.6 Hz 2H), 5.42 (d, J=11.8, 1H), 3.93 (dq, J=13.7, 7.01 Hz, 1H), 3.70 (t, J=4.68, 1H), 3.56 (dt, J=5.29, 5.95 Hz, 1H), 3.37 (m, 1H) 2.60 (m, 3H), 2.48 (m, 3H), 2.48 (s, 3H), 2.38 (s, 3H), 2.22 (s, 3H), 1.46 (d, J=6.8 Hz, 3H).

### Example 85: 5-((1R,2S)-1-(6-chloro-4-(2,3-dihydroxypropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

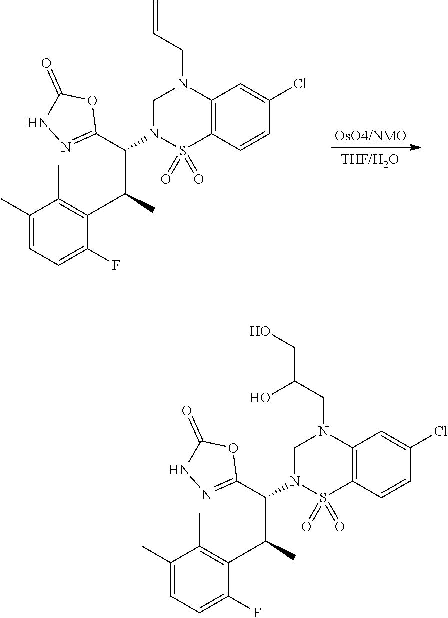 [SMILES: C=CCN1CN([C@@H](c2n[nH]c(=O)o2)[C@@H](C)c2c(F)ccc(C)c2C)S(=O)(=O)c2ccc(Cl)cc21 ; Cc1ccc(F)c([C@H](C)[C@H](c2n[nH]c(=O)o2)N2CN(CC(O)CO)c3cc(Cl)ccc3S2(=O)=O)c1C]

5-((1R,2S)-1-(4-allyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one was synthesized following the procedure used for example 30. For step 1, allylamine was used instead of 2-methoxypropylamine.

Then a solution of 5-((1R,2S)-1-(4-allyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (50 mg, 0.094 mmol, 1.0 eq) in THF (1.5 ml) and water (0.5 mL) were added 4% aqueous solution of osmium tetroxide (4 mol %) NMMO (2 eq) and the stirring was continued overnight. The reaction was quenched with sat. NaHSO₃ solution and extracted with DCM. Combined organic fractions were dried over Na₂SO₄ and concentrated. The residue was purified by pTLC to afford the title compound [00490]5-((1R,2S)-1-(6-chloro-4-(2,3-dihydroxypropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (48 mg, 0.086 mmol, yield 92%) as a mixture of diastereomers.

¹H NMR (300 MHz, Methanol-d₄) δ 7.62-7.51 (m, 1H), 7.03-6.90 (m, 2H), 6.82-6.66 (m, 2H), 5.69-5.36 (m, 2H), 5.11 (dd, J=38.8, 15.2 Hz, 1H), 3.98-3.80 (m, 2H), 3.78-3.61 (m, 1H), 3.58-3.43 (m, 3H), 3.42-3.35 (m, 1H), 3.10-2.98 (m, 1H), 2.41-2.34 (m, 3H), 2.22 (s, 3H), 1.45 (d, J=6.7 Hz, 3H). LC-MS: 539.17 [M−H]−.

### Example 86: 5-((1S,2R)-1-(6-chloro-4-((S)-1-methylpyrrolidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound (10 mg, yield 9%) was synthesized following the procedure used for example 30. (3S)-1-methylpyrrolidin-3-amine was used instead of 2-methoxypropylamine. Step 1 was carried out at 95° C. for 3 days. ¹H NMR (400 MHz, Methanol-d₄) δ 7.58 (d, J=8.4 Hz, 1H), 7.13 (d, J=1.8 Hz, 1H), 7.04-6.96 (m, 1H), 6.82 (dd, J=8.4, 1.8 Hz, 1H), 6.73 (dd, J=12.2, 8.3 Hz, 1H), 5.43 (dd, J=11.6, 1.6 Hz, 1H), 5.30 (s, 2H), 4.49-4.43 (m, 1H), 4.02-3.90 (m, 1H), 3.19-3.12 (m, 1H), 3.00 (dd, J=11.0, 4.5 Hz, 1H), 2.76-2.69 (m, 1H), 2.59-2.53 (m, 1H), 2.51 (s, 3H), 2.38 (s, 3H), 2.33-2.27 (m, 1H), 2.22 (s, 3H), 2.20-2.16 (m, 1H), 1.47 (dd, J=7.0, 1.1 Hz, 3H). LC-MS: m/z=550.23 [M+H]⁺; 548.33 [M−H]−.

### Example 87: 5-((1S,2R)-1-(6-chloro-1,1-dioxido-4-(tetrahydro-2H-pyran-4-yl)-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

The title compound was synthesized following the procedure used for example 48. For Step 1, 4-aminotetrahydropyrane was used instead of 3-methoxypropylamine. Step 2 was carried out at 80° C. with excess of 4M HCl (8 eq) and paraformaldehyde (20 eq). ¹H NMR (400 MHz, Methanol-d₄) δ 7.88 (d, J=8.0 Hz, 1H), 7.00 (dd, J=8.4, 5.7 Hz, 1H), 6.78 (d, J=8.0 Hz, 1H), 6.72 (dd, J=12.2, 8.4 Hz, 1H), 5.42 (dd, J=11.9, 2.1 Hz, 1H), 5.29 (d, J=14.6 Hz, 1H), 5.23 (d, J=14.6 Hz, 1H), 4.72 (tt, J=12.1, 4.0 Hz, 1H), 4.06 (ddd, J=20.0, 11.4, 4.3 Hz, 2H), 4.01-3.93 (m, 1H), 3.53 (dtd, J=15.7, 11.9, 2.1 Hz, 2H), 2.38 (s, 3H), 2.22 (s, 3H), 2.17-2.06 (m, 1H), 2.00 (qd, J=12.2, 4.6 Hz, 1H), 1.78-1.70 (m, 2H), 1.46 (dd, J=6.9, 1.1 Hz, 3H). LC-MS: m/z=552.25 [M+H]+; 550.25 [M−H]−.

### Example 88: 5-((1S,2R)-1-(6-chloro-4-(2,2-difluoroethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 30. For Step 1, 2,2-difluoroethylamine (60 eq) was used instead of 2-methoxypropylamine and the reaction was carried out at 105° C. for 4 days. ¹H NMR (400 MHz, Methanol-d₄) δ 7.58 (d, J=8.4 Hz, 1H), 6.93 (dd, J=8.4, 5.7 Hz, 1H), 6.86 (s, 1H), 6.82 (dd, J=8.4, 1.8 Hz, 1H), 6.67 (dd, J=12.0, 8.4 Hz, 1H), 6.09 (tdd, J=55.1, 3.9, 2.6 Hz, 1H), 5.51 (d, J=14.6 Hz, 1H), 5.47 (d, J=11.8 Hz, 1H), 5.14 (d, J=14.6 Hz, 1H), 4.03-3.93 (m, 1H), 3.93-3.85 (m, 1H), 3.55 (ddd, J=16.9, 12.8, 4.0 Hz, 1H), 2.38 (s, 3H), 2.20 (s, 3H), 1.44 (dd, J=6.9, 1.2 Hz, 3H). ¹⁹F NMR (376 MHz, Methanol-d₄) δ −117.55 (dd, J=12.7, 5.5 Hz), −122.11-−122.60 (m). LC-MS: m/z=529.14 [M−H]−.

### Examples 89-90: 5-((1S,2R)-1-(6-chloro-4-((R)-2,2-dimethyltetrahydro-2H-pyran-4-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one and 5-((1S,2R)-1-(6-chloro-4-((S)-2,2-dimethyltetrahydro-2H-pyran-4-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compounds were synthesized following the procedure used for example 30. For Step 1, 2-methoxypropylamine was substituted with 2,2-dimethyltetrahydro-2H-pyran-4-amine. Diastereomeric separation was done by pTLC (the stereochemistry was arbitrarily assessed).

Diastereomer 1 (89): ¹H NMR (400 MHz, Methanol-d₄) δ 7.57 (d, J=8.4 Hz, 1H), 6.99 (dd, J=8.5, 5.7 Hz, 1H), 6.88 (d, J=1.8 Hz, 1H), 6.79 (dd, J=8.4, 1.8 Hz, 1H), 6.72 (dd, J=12.1, 8.4 Hz, 1H), 5.40 (dd, J=11.9, 1.7 Hz, 1H), 5.21 (d, J=14.3 Hz, 1H), 5.17 (d, J=14.3 Hz, 1H), 4.16-4.06 (m, 1H), 3.93 (dq, J=14.4, 7.8, 7.3 Hz, 1H), 3.87-3.80 (m, 2H), 2.38 (s, 3H), 2.22 (s, 3H), 1.92-1.77 (m, 4H), 1.46 (dd, J=7.0, 1.1 Hz, 3H), 1.38 (s, 3H), 1.33 (s, 3H). LC-MS: m/z=577.24 [M−H]−.

Diastereomer 2 (90): ¹H NMR (400 MHz, Methanol-d₄) δ7.57 (d, J=8.4 Hz, 1H), 6.99 (dd, J=8.5, 5.7 Hz, 1H), 6.93 (d, J=1.8 Hz, 1H), 6.80 (dd, J=8.4, 1.8 Hz, 1H), 6.72 (dd, J=12.1, 8.4 Hz, 1H), 5.42 (dd, J=11.8, 1.7 Hz, 1H), 5.20 (d, J=14.3 Hz, 1H), 5.16 (d, J=14.4 Hz, 1H), 4.16-4.07 (m, 1H), 3.98-3.90 (m, 1H), 3.89 (dd, J=8.5, 2.1 Hz, 2H), 2.37 (s, 3H), 2.22 (s, 3H), 2.07-1.99 (m, 1H), 1.90-1.83 (m, 1H), 1.77-1.71 (m, 1H), 1.67 (d, J=12.5 Hz, 1H), 1.46 (dd, J=6.9, 1.1 Hz, 3H), 1.34 (s, 3H), 1.26 (s, 3H). LC-MS: m/z=577.22 [M−H]−.

### Example 91: 5-((1S,2R)-1-(6-chloro-4-(2-hydroxy-2-methylpropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 30. For Step 1, 2-methoxypropylamine was substituted with 1-amino-2-methylpropan-2-ol. ¹H NMR (400 MHz, Methanol-d₄) δ 7.57 (d, J=8.5 Hz, 1H), 6.99 (dd, J=8.4, 5.7 Hz, 1H), 6.94 (d, J=1.8 Hz, 1H), 6.76 (dd, J=8.4, 1.8 Hz, 1H), 6.73-6.68 (m, 1H), 5.65 (d, J=15.1 Hz, 1H), 5.39 (dd, J=11.8, 1.6 Hz, 1H), 5.09 (d, J=15.1 Hz, 1H), 3.95-3.85 (m, 1H), 3.64 (d, J=15.5 Hz, 1H), 3.07 (d, J=15.6 Hz, 1H), 2.38 (s, 3H), 2.22 (s, 3H), 1.45 (dd, J=6.9, 1.1 Hz, 3H), 1.29 (s, 3H), 1.16 (s, 3H). LC-MS: m/z=539.16 [M+H]⁺; 537.15 [M−H]−.

### Example 92: 5-((1S,2R)-1-(6-chloro-1,1-dioxido-4-(2-(pyrrolidin-1-yl)ethyl)-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

The title compound was synthesized following the procedure used for example 48. For Step 1, N-(2-aminoethyl)pyrrolidine was used instead of 3-methoxypropylamine. ¹H NMR (400 MHz, Methanol-d₄) δ 7.87 (d, J=8.0 Hz, 1H), 6.95 (dd, J=8.4, 5.7 Hz, 1H), 6.74 (d, J=8.0 Hz, 1H), 6.67 (dd, J=12.1, 8.4 Hz, 1H), 5.46-5.39 (m, 2H), 5.22 (d, J=14.6 Hz, 1H), 4.11 (dt, J=14.0, 6.9 Hz, 1H), 3.98-3.86 (m, 1H), 3.37-3.33 (m, 1H), 2.87-2.79 (m, 2H), 2.79-2.75 (m, 4H), 2.39 (s, 3H), 2.21 (s, 3H), 1.85 (q, J=3.4, 3.0 Hz, 4H), 1.45 (dd, J=6.9, 1.1 Hz, 3H). LC-MS: m/z=565.23 [M+H]*.

### Example 93: 5-((1S,2R)-1-(6-chloro-4-(2-morpholinoethyl)-1,1-dioxido-3,4-dihydro-2H-pyrido[2,3-e][1,2,4]thiadiazin-2-yl)-2-(6-fluoro-2,3-dimethylphenyl)propyl)-1,3,4-oxadiazol-2 (3H)-one

The title compound was synthesized following the procedure used for example 48. For Step 1, 4-(2-aminoethyl) morpholine was used instead of 3-methoxypropylamine. ¹H NMR (400 MHz, Methanol-d₄) δ 7.89 (d, J=8.0 Hz, 1H), 7.00 (dd, J=8.4, 5.7 Hz, 1H), 6.77 (d, J=8.0 Hz, 1H), 6.72 (dd, J=12.1, 8.4 Hz, 1H), 5.49 (d, J=14.8 Hz, 1H), 5.41 (dd, J=11.7, 1.8 Hz, 1H), 5.21 (d, J=14.8 Hz, 1H), 4.16 (dt, J=14.3, 6.3 Hz, 1H), 3.95 (dq, J=13.2, 6.9 Hz, 1H), 3.65 (q, J=4.3 Hz, 4H), 3.18 (dt, J=13.8, 6.5 Hz, 1H), 2.60 (td, J=12.4, 6.2 Hz, 2H), 2.55-2.47 (m, 4H), 2.39 (s, 3H), 2.22 (s, 3H), 1.46 (dd, J=6.9, 1.1 Hz, 3H). LC-MS: m/z=581.27 [M+H]*.

### Example 94 and 95: 5-((1S,2S)-1-(8-acetyl-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one and 5-((1S,2R)-1-(8-acetyl-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

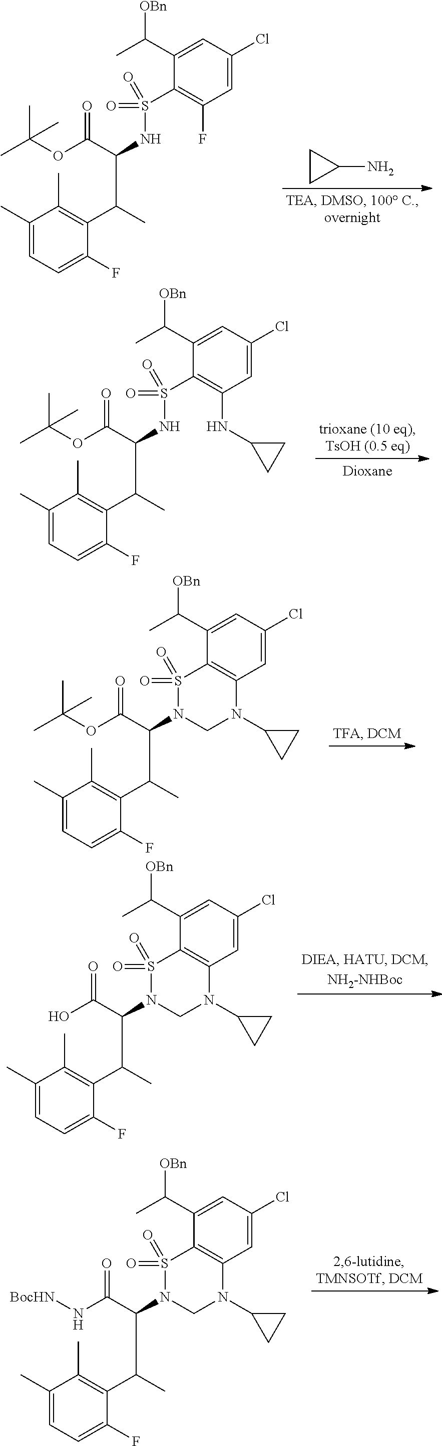

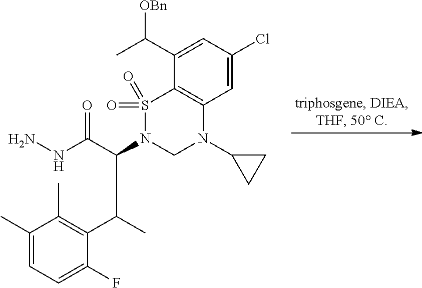 [SMILES: Cc1ccc(F)c(C(C)[C@@H](C(=O)NN)N2CN(C3CC3)c3cc(Cl)cc(C(C)OCc4ccccc4)c3S2(=O)=O)c1C]

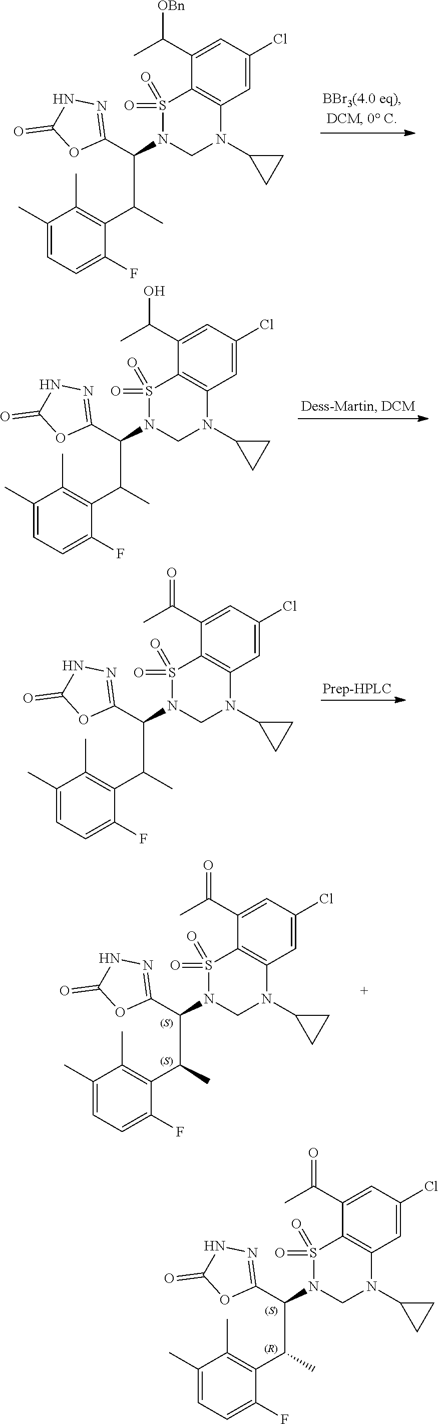

### Step 1: Synthesis of tert-butyl (2S)-2-((2-(1-(benzyloxy) ethyl)-4-chloro-6-(cyclopropylamino)phenyl)sulfonamido)-3-(6-fluoro-2,3-dimethylphenyl)butanoate

Into a 40 mL sealed tube were added tert-butyl (2S)-2-(2-[1-(benzyloxy)ethyl]-4-chloro-6-fluorobenzenesulfonamido-3-(6-fluoro-2,3-dimethylphenyl)butanoate (see ex 68 step 5) (380 mg, 0.625 mmol, 1 equiv), DMSO (7.60 mL), TEA (868.56 μL, 6.25 mmol, 10 equiv) and aminocyclopropane (649.5 μL, 9.38 mmol, 15 equiv) at room temperature. The resulting mixture was stirred overnight at room temperature under air atmosphere. This resulted in tert-butyl (2S)-2-(2-[1-(benzyloxy) ethyl]-4-chloro-6-(cyclopropylamino) benzenesulfonamido-3-(6-fluoro-2,3-dimethylphenyl) butanoate (327 mg, 81.1%) as a yellow solid.

### Step 2: tert-butyl (2S)-2-(8-(1-(benzyloxy) ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate

Into a 30 mL sealed tube were added tert-butyl (2S)-2-(2-[1-(benzyloxy)ethyl]-4-chloro-6-(cyclopropylamino)benzenesulfonamido-3-(6-fluoro-2,3-dimethylphenyl)butanoate (300 mg, 0.466 mmol, 1 equiv) and dioxane (6 mL), trioxane (418.8 mg, 4.65 mmol, 10 equiv) and para-toluene sulfonate (40 mg, 0.23 mmol, 0.5 equiv) at room temperature. The resulting mixture was stirred overnight at 100° C. under air atmosphere. The resulting mixture was concentrated under reduced pressure. The residue was purified by reversed-phase flash chromatography with the following conditions: column, C18 silica gel; mobile phase, MeCN in Water (0.1% FA), 60% to 70% gradient in 10 min; detector, UV 254 nm. This resulted in tert-butyl (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate (270 mg, 88.4%) as a yellow solid.

### Step 3: Synthesis of (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid

Into a 50 mL round-bottom flask were added tert-butyl (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate (160 mg, 0.243 mmol, 1 equiv), DCM (6.24 mL) and TFA (3.12 mL) at room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid (271 mg, 89%) as a brown solid.

### Step 4: Synthesis of tert-butyl 2-((2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoyl)hydrazine-1-carboxylate

Into a 10 mL vial were added (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid (150 mg, 0.250 mmol, 1 equiv), DCM (3 mL), DIEA (96.8 mg, 0.75 mmol, 3 equiv), tert-butoxycarbohydrazide (49.47 mg, 0.375 mmol, 1.5 equiv) and HATU (94.88 mg, 0.250 mmol, 1 equiv) at room temperature. The resulting mixture was stirred for 0.5 h at room temperature under air atmosphere. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (100 mg, 66.9%) as a white solid.

### Step 5: (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide

Into a 50 mL round-bottom flask were added tert-butyl 2-((2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoyl)hydrazine-1-carboxylate (131 mg, 0.183 mmol, 1 equiv), DCM (1.5 mL) and lutidine (426.63 μL, 3.660 mmol, 20 equiv) at room temperature. To the above mixture was added TMSOTf (530.35 μL, 2.928 mmol, 16 equiv) dropwise at 0° C. The resulting mixture was stirred for additional 1 h at room temperature. The resulting mixture was concentrated under reduced pressure. The residue was purified by reversed-phase flash chromatography with the following conditions: column, C18 silica gel; mobile phase, MeCN in Water (0.1% FA), 70% to 80% gradient in 10 min; detector, UV 254 nm. This resulted in (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide (100 mg, 88.8%) as a white solid.

### Step 6: Synthesis of 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 40 mL sealed tube were (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide (572 mg, 0.930 mmol, 1 equiv), THF (11.44 mL), DIEA (404.92 μL, 2.325 mmol, 2.5 equiv) and triphosgene (137.96 mg, 0.465 mmol, 0.5 equiv) at room temperature. The resulting mixture was stirred for 0.5 h at 50° C. under air atmosphere. The residue was purified by reversed-phase flash chromatography with the following conditions: column, C18 silica gel; mobile phase, MeCN in Water (0.1% FA), 95% to 100% gradient in 10 min; detector, UV 254 nm. This resulted in 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (372 mg, 62.4%) as a white solid.

### Step 7: Synthesis of 5-((1S)-1-(6-chloro-4-cyclopropyl-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (362 mg, 0.565 mmol, 1 equiv) and DCM (1.6 mL) at room temperature. To the above mixture was added BBr₃ (2.26 mL, 2.260 mmol, 4 equiv) dropwise at 0° C. The resulting mixture was stirred for additional 0.5 h at 0° C. The reaction was quenched by the addition of sat. sodium hyposulfite (aq.) (5 mL) at 0° C. The resulting mixture was extracted with CH₂Cl₂ (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 70% to 74% gradient in 10 min; detector, UV 254 nm. This resulted in 5-((1S)-1-(6-chloro-4-cyclopropyl-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (131 mg, 42.11%) as a white solid.

### Step 8: Synthesis of 5-((1S,2S)-1-(8-acetyl-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one and 5-[(1S,2R)-1-(8-acetyl-6-chloro-4-cyclopropyl-1,1-dioxo-3H-1λ6,2,4-benzothiadiazin-2-yl)-2-(6-fluoro-2,3-dimethyl-phenyl)propyl]-3H-1,3,4-oxadiazol-2-one

Into a 10 mL vial were added 5-((1S)-1-(6-chloro-4-cyclopropyl-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (131 mg, 0.238 mmol, 1 equiv), DCM (3 mL) and dess-martin reagent (201.67 mg, 0.476 mmol, 2 equiv) at room temperature. The resulting mixture was stirred for 0.5 h at room temperature under air atmosphere. The resulting mixture was extracted with EtOAc (2×50 mL). The combined organic layers were washed with brine (1×50 mL), dried over anhydrous Na₂SO₄. After filtration, the filtrate was concentrated under reduced pressure. The crude product was purified by Prep-HPLC with the following conditions: Column: YMC-Actus Triart C18 ExRS Column, 30*150 mm, 5 μm; Mobile Phase A: Water (10 mmol/L NH₄HCO₃+0.1% NH₃·H₂O), Mobile Phase B: ACN; Flow rate: 60 mL/min mL/min; Gradient: 28% B to 44% B in 7 min; Wave Length: 254 nm/220 nm nm; The 2 diastereoisomers were isolated:

94: 5-((1S,2S)-1-(8-acetyl-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (29.2 mg, 17.56%). LCMS:(ES,m/z): [M−H]+=547.35. ¹H NMR (400 MHz, Methanol-d₄) δ 7.28 (d, J=1.9 Hz, 1H), 7.09 (dd, J=8.4, 5.7 Hz, 1H), 6.89-6.77 (m, 2H), 5.26 (d, J=11.5 Hz, 1H), 4.91 (s, 1H), 4.78 (d, J=14.2 Hz, 1H), 4.03 (dq, J=13.6, 7.0 Hz, 1H), 2.59 (s, 4H), 2.40 (s, 3H), 2.31 (s, 3H), 1.26 (d, J=6.8 Hz, 3H), 1.02 (dd, J=7.6, 4.9 Hz, 2H), 0.84 (dd, J=10.5, 4.1 Hz, 1H), 0.76-0.68 (m, 1H).

95: 5-((1S,2R)-1-(8-acetyl-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (37.0 mg, 24.15%). LCMS:(ES,m/z): [M+H]*=549.10. ¹H NMR (400 MHz, Methanol-d₄) δ 7.25 (d, J=1.9 Hz, 1H), 6.98 (dd, J=8.4, 5.7 Hz, 1H), 6.86 (d, J=1.9 Hz, 1H), 6.71 (dd, J=12.1, 8.4 Hz, 1H), 5.39-5.26 (m, 2H), 5.18 (d, J=14.5 Hz, 1H), 3.93 (dtd, J=10.5, 7.4, 5.8 Hz, 1H), 2.60 (s, 3H), 2.50 (tt, J=6.9, 3.8 Hz, 1H), 2.36 (s, 3H), 2.21 (s, 3H), 1.43 (dd, J=7.0, 1.2 Hz, 3H), 1.09-0.87 (m, 3H), 0.67 (dtd, J=9.8, 5.8, 4.8, 2.6 Hz, 1H).

### Example 96: 5-((1S,2R)-1-(6-chloro-1,1-dioxido-4-(1-(2,2,2-trifluoroethyl)azetidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

2,2,2-Trifluoroethyl trifluoromethanesulfonate (20 mg, 0.085 mmol, 1 eq) was added to a solution of 5-((1S,2R)-1-(4-(azetidin-3-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (see example 57) (50 mg, 0.085 mmol, 1.0 eq) and N,N-Diisopropylethylamine (DIPEA) (0.075 mL, 0.43 mmol, 5 eq) in anhydrous DMF (0.5 mL).

The reaction was carried out at RT for 2 h. Upon completion, the mixture was diluted with water and extracted with AcOEt. Organic layer was dried over Na₂SO₄, filtered and concentrated. The residue was purified by pHPLC to afford 6-chloro-2-[(1S,2R)-2-(6-fluoro-2,3-dimethylphenyl)-1-(5-oxo-4,5-dihydro-1,3,4-oxadiazol-2-yl)propyl]-4-[1-(2,2,2-trifluoroethyl)azetidin-3-yl]-3,4-dihydro-2H-17′,2,4-benzothiadiazine-1,1-dione. ¹H NMR (400 MHz, Methanol-d₄) δ 7.61 (d, J=8.4 Hz, 1H), 7.00 (dd, J=8.4, 5.7 Hz, 1H), 6.88 (dd, J=8.5, 1.8 Hz, 1H), 6.73 (dd, J=12.1, 8.4 Hz, 1H), 6.62 (d, J=1.8 Hz, 1H), 5.44 (d, J=11.7 Hz, 1H), 5.34 (d, J=14.4 Hz, 1H), 5.20 (d, J=14.4 Hz, 1H), 4.23 (p, J=6.9 Hz, 1H), 3.98-3.84 (m, 3H), 3.64 (t, J=7.2 Hz, 1H), 3.37 (t, J=7.1 Hz, 1H), 3.25 (q, J=9.7 Hz, 2H), 2.38 (s, 3H), 2.23 (s, 3H), 1.47 (d, J=6.8 Hz, 3H). 19F NMR (376 MHz, Methanol-d₄) δ −72.56 (t, J=9.6 Hz), −118.01 (dd, J=12.6, 5.6 Hz).

### Example 97: 5-((1S,2R)-1-(6-chloro-4-((3S,4R)-3-fluorotetrahydro-2H-pyran-4-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 30. For Step 1, trans-3-fluorooxan-4-amine was used instead of 3-methoxypropylamine. Step 1 was carried out at 95° C. for 2 days. Step 2 was carried out at 95° C. for 2 days. ¹H NMR (400 MHz, Methanol-d₄) δ 7.91 (dd, J=8.0, 5.8 Hz, 2H), 7.00 (dd, J=8.5, 5.7 Hz, 2H), 6.82 (t, J=8.1 Hz, 2H), 6.73 (dd, J=12.2, 8.4 Hz, 2H), 5.49 (dd, J=11.9, 2.2 Hz, 1H), 5.46-5.41 (m, 1H), 5.41-5.34 (m, 2H), 5.30 (dd, J=14.5, 1.9 Hz, 2H), 5.16-5.09 (m, 1H), 5.06-4.97 (m, 1H), 4.93-4.89 (m, 1H), 4.32 (s, 1H), 4.22-4.16 (m, 2H), 4.06-3.94 (m, 4H), 3.50 (dtd, J=31.2, 11.9, 2.1 Hz, 3H), 3.38-3.33 (m, 2H), 2.59-2.47 (m, 2H), 2.38 (d, J=5.6 Hz, 6H), 2.22 (s, 6H), 1.91-1.82 (m, 2H), 1.45 (dt, J=6.9, 1.3 Hz, 6H). LC-MS: m/z=568.13 [M−H]−.

### Example 98: 5-((1S,2R)-1-(6-chloro-4-(2-methoxy-2-methylpropyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the method used for example 30. For Step 1, 2-methoxyethylamine was substituted with 2-methoxy-2-methylpropan-1-amine.

¹H NMR (400 MHz, Methanol-d₄) δ 7.56 (d, J=8.5 Hz, 1H), 7.01-6.98 (m, 1H), 6.97 (d, J=1.8 Hz, 1H), 6.75 (dd, J=8.4, 1.7 Hz, 1H), 6.73-6.68 (m, 1H), 5.54 (d, J=15.1 Hz, 1H), 5.38 (dd, J=11.8, 1.6 Hz, 1H), 5.02 (d, J=15.2 Hz, 1H), 3.93-3.84 (m, 1H), 3.64 (d, J=15.6 Hz, 1H), 3.25 (s, 3H), 3.11 (d, J=15.7 Hz, 1H), 2.37 (s, 3H), 2.22 (s, 3H), 1.45 (dd, J=6.9, 1.1 Hz, 3H), 1.24 (s, 3H), 1.13 (s, 3H). LC-MS: m/z=553.23 [M+H]+; 551.20 [M−H]−.

### Example 99: 5-((1S,2R)-1-(6-chloro-1,1-dioxido-4-((R)-1-(2,2,2-trifluoroethyl)pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the method used for example 30. For Step 1, (R)-1-(2,2,2-trifluoroethyl)pyrrolidin-3-amine was used instead of 3-methoxypropylamine. ¹H NMR (400 MHz, Methanol-d₄) δ 7.59 (d, J=8.5 Hz, 1H), 7.10 (d, J=1.9 Hz, 1H), 7.01 (dd, J=8.4, 5.7 Hz, 1H), 6.84 (dd, J=8.4, 1.8 Hz, 1H), 6.74 (dd, J=12.2, 8.4 Hz, 1H), 5.48 (dd, J=11.9, 2.2 Hz, 1H), 5.25 (d, J=14.8 Hz, 1H), 5.19 (d, J=14.8 Hz, 1H), 4.44-4.37 (m, 1H), 3.89 (dq, J=13.6, 7.2 Hz, 1H), 3.51-3.45 (m, 1H), 3.42-3.33 (m, 1H), 3.29-3.15 (m, 2H), 2.71 (dd, J=11.1, 7.2 Hz, 1H), 2.52 (q, J=8.8 Hz, 1H), 2.37 (s, 3H), 2.31 (ddd, J=12.2, 8.4, 2.9 Hz, 1H), 2.23 (s, 3H), 1.79 (dtd, J=13.3, 8.7, 4.4 Hz, 1H), 1.45 (dd, J=7.0, 1.1 Hz, 3H). LC-MS: m/z=618.15 [M+H]+; 616.14 [M−H]−.

### Example 100: 5-((1S,2R)-1-(6-chloro-8-(1-chloroethyl)-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

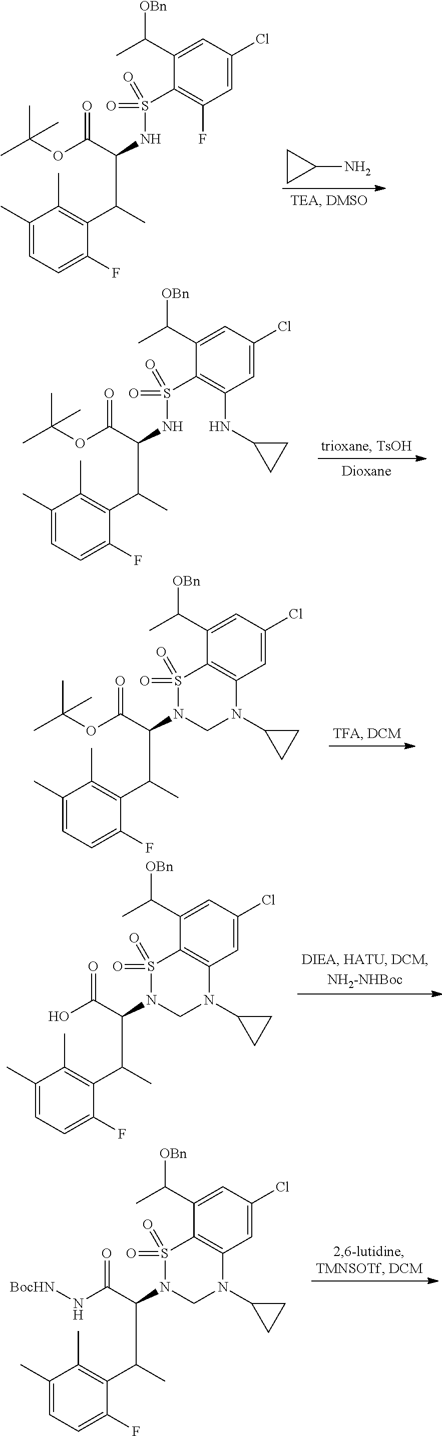

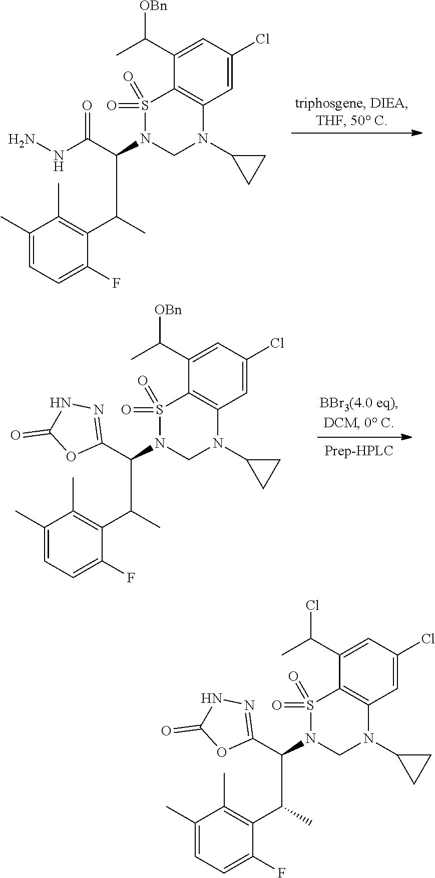 [SMILES: Cc1ccc(F)c(C(C)[C@@H](C(=O)NN)N2CN(C3CC3)c3cc(Cl)cc(C(C)OCc4ccccc4)c3S2(=O)=O)c1C ; Cc1ccc(F)c(C(C)[C@@H](c2n[nH]c(=O)o2)N2CN(C3CC3)c3cc(Cl)cc(C(C)OCc4ccccc4)c3S2(=O)=O)c1C ; Cc1ccc(F)c([C@@H](C)[C@@H](c2n[nH]c(=O)o2)N2CN(C3CC3)c3cc(Cl)cc(C(C)Cl)c3S2(=O)=O)c1C]

### Step 1: Synthesis of tert-butyl (2S)-2-((2-(1-(benzyloxy)ethyl)-4-chloro-6-(cyclopropylamino)phenyl)sulfonamido)-3-(6-fluoro-2,3-dimethylphenyl)butanoate

Into a 40 mL sealed tube were added tert-butyl (2S)-2-(2-[1-(benzyloxy)ethyl]-4-chloro-6-fluorobenzenesulfonamido-3-(6-fluoro-2,3-dimethylphenyl)butanoate (see ex. 68×380 mg, 0.625 mmol, 1 equiv), DMSO (7.60 mL), TEA (868.56 μL, 6.250 mmol, 10 equiv) and aminocyclopropane (649.46 μL, 9.375 mmol, 15 equiv) at room temperature. The resulting mixture was stirred overnight at room temperature under air atmosphere. The resulting mixture was diluted with H₂O (50 ml), extracted with EtOAc (2×50 mL). The combined organic layers were washed with brine (1×50 mL), dried over anhydrous Na₂SO₄. After filtration, the filtrate was concentrated under reduced pressure. This resulted in tert-butyl (2S)-2-(2-[1-(benzyloxy)ethyl]-4-chloro-6-(cyclopropylamino)benzenesulfonamido-3-(6-fluoro-2,3-dimethylphenyl)butanoate (327 mg, 81.1%) as a yellow solid.

### Step 2: Synthesis of tert-butyl (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate

Into a 30 mL sealed tube were added tert-butyl (2S)-2-(2-[1-(benzyloxy)ethyl]-4-chloro-6-(cyclopropylamino)benzenesulfonamido-3-(6-fluoro-2,3-dimethylphenyl)butanoate (300 mg, 0.466 mmol, 1 equiv) and dioxane (6 mL), trioxane (418.82 mg, 4.650 mmol, 10 equiv) and para-toluene sulfonate (40.03 mg, 0.233 mmol, 0.5 equiv) at room temperature. The resulting mixture was stirred overnight at 100° C. under air atmosphere. The resulting mixture was concentrated under reduced pressure. The residue was purified by reversed-phase flash chromatography with the following conditions: column, C18 silica gel; mobile phase, MeCN in Water (0.1% FA), 60% to 70% gradient in 10 min; detector, UV 254 nm. This resulted in tert-butyl (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate (270 mg, 88.4%) as a yellow solid.

### Step 3: Synthesis of (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid

Into a 50 mL round-bottom flask were added in tert-butyl (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoate (160 mg, 0.243 mmol, 1 equiv), DCM (6.24 mL) and TFA (3.12 mL) at room temperature. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoic acid. (271 mg, 89%) as a brown solid.

### Step 4: Synthesis of tert-butyl 2-((2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoyl)hydrazine-1-carboxylate

Into a 10 mL vial were added (2S)-2-(8-[1-(benzyloxy)ethyl]-6-chloro-4-cyclopropyl-1,1-dioxo-3H-1lambda6,2,4-benzothiadiazin-2-yl-3-(6-fluoro-2,3-dimethylphenyl)butanoic acid (150 mg, 0.250 mmol, 1 equiv), DCM (3 mL), DIEA (96.75 mg, 0.750 mmol, 3 equiv), tert-butoxycarbohydrazide (49.47 mg, 0.375 mmol, 1.5 equiv) and HATU (94.88 mg, 0.250 mmol, 1 equiv) at room temperature. The resulting mixture was stirred for 0.5 h at room temperature under air atmosphere. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in (100 mg, 66.9%) of tert-butyl 2-((2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoyl)hydrazine-1-carboxylate as a white solid.

### Step 5: Synthesis of (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide

Into a 50 mL round-bottom flask were added tert-butyl 2-((2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanoyl)hydrazine-1-carboxylate (131 mg, 0.183 mmol, 1 equiv), DCM (1.5 mL) and lutidine (426.63 μL, 3.660 mmol, 20 equiv) at room temperature. To the above mixture was added TMSOTf (530.35 μL, 2.928 mmol, 16 equiv) dropwise at 0° C. The resulting mixture was stirred for additional 1 h at room temperature. The resulting mixture was concentrated under reduced pressure. The residue was purified by reversed-phase flash chromatography with the following conditions: column, C18 silica gel; mobile phase, MeCN in Water (0.1% FA), 70% to 80% gradient in 10 min; detector, UV 254 nm. This resulted in (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide (100 mg, 88.76%) as a white solid.

### Step 6: Synthesis of 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 40 mL sealed tube were added (2S)-2-(8-(1-(benzyloxy)ethyl)-6-chloro-4-cyclopropyl-1,1-dioxido-3,4-dihydro-2H-benzo[e][1,2,4]thiadiazin-2-yl)-3-(6-fluoro-2,3-dimethylphenyl)butanehydrazide (572 mg, 0.930 mmol, 1 equiv), THF (11.44 mL), DIEA (404.92 μL, 2.325 mmol, 2.5 equiv) and triphosgene (137.96 mg, 0.465 mmol, 0.5 equiv) at room temperature. The resulting mixture was stirred for 0.5 h at 50° C. under air atmosphere. The residue was purified by reversed-phase flash chromatography with the following conditions: column, C18 silica gel; mobile phase, MeCN in Water (0.1% FA), 95% to 100% gradient in 10 min; detector, UV 254 nm. This resulted in of 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (372 mg, 62.4%) as a white solid.

### Step 7: Synthesis of 5-((1S,R)-1-(6-chloro-8-(1-chloroethyl)-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added 5-((1S)-1-(8-(1-(benzyloxy)ethyl)-6-chloro-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (362 mg, 0.565 mmol, 1 equiv) and DCM (1.6 mL) at room temperature. To the above mixture was added BCl3 (2.26 mL 1M in DCM, 4 equiv) dropwise at 0° C. The resulting mixture was stirred for additional 0.5 h at 0° C. The reaction was quenched by the addition of sat. sodium hyposulfite (aq.) (5 mL) at 0° C. The resulting mixture was extracted with CH₂Cl₂ (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 70% to 74% gradient in 10 min; detector, UV 254 nm. This resulted in 5-((1S)-1-(6-chloro-8-(1-chloroethyl)-4-cyclopropyl-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (100 mg, 31.15%).

The mixture of diastereoisomers was fractionated by Prep-HPLC with the following conditions: Column: XselectCSH Prep OBD C18 Column, 30*150 mm, 5 μm; Mobile Phase A: Water (0.1% FA), Mobile Phase B: ACN; Flow rate: 60 mL/min mL/min; Gradient: 62% B to 72% B in 9 min; Wave Length: 254 nm/220 nm nm; RT1 (min): 7.80; 8.52. 6-chloro-8-(1-chloroethyl)-4-cyclopropyl-2-[(1S)-2-(6-fluoro-2,3-dimethylphenyl)-1-(5-oxo-4H-1,3,4-oxadiazol-2-yl)propyl]-3H-1lambda6,2,4-benzothiadiazine-1,1-dione was isolated (14 mg, 13.9%). LCMS:(ES,m/z): [M−H]⁺=567.20. ¹H NMR (400 MHz, DMSO-d₆) δ 12.22 (s, 1H), 7.20 (ddd, J=16.3, 7.4, 2.0 Hz, 2H), 7.07-6.99 (m, 1H), 6.83 (ddd, J=12.1, 8.3, 3.6 Hz, 1H), 5.96 (dq, J=25.3, 6.6 Hz, 1H), 5.42 (dd, J=11.9, 2.0 Hz, 1H), 5.24-5.13 (m, 1H), 5.10 (dd, J=14.8, 2.1 Hz, 1H), 3.97 (dt, J=12.7, 7.3 Hz, 1H), 2.40 (dq, J=6.8, 3.3 Hz, 1H), 2.33 (d, J=4.3 Hz, 3H), 2.19 (s, 3H), 1.82 (dd, J=15.9, 6.6 Hz, 3H), 1.32 (d, J=6.8 Hz, 3H), 0.98 (td, J=6.6, 4.2 Hz, 1H), 0.88 (s, 1H), 0.64 (ddq, J=23.8, 10.0, 4.5 Hz, 2H).

### Example 101: 5-((1S,2R)-1-(6-chloro-1,1-dioxido-4-((S)-1-(2,2,2-trifluoroethyl)pyrrolidin-3-yl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure described for example 30. For Step 1 (S)-1-(2,2,2-trifluoroethyl)pyrrolidin-3-amine was used instead of 3-methoxypropylamine.

¹H NMR (400 MHz, Methanol-d₄) δ 7.57 (d, J=8.4 Hz, IM) 7.21 (d, J=1.8 Hz, 1H), 7.00 (dd, J=8.4, 5.8 Hz, 1H), 6.80 (dd, J=8.4, 1.8 Hz, 1H), 6.73 (dd, J=12.1, 8.4 Hz, 1H), 5.43 (dd, J=11.9, 1.9 Hz, 1H), 5.38 (d, J=14.5 Hz, 1H), 5.33 (d, J=14.5 Hz, 1H), 4.35-4.26 (m, 1H), 3.99-3.86 (m, 1H), 3.37-3.33 (m, 1H), 3.28-3.20 (m, 2H), 3.19-3.16 (m, 1H), 2.68 (dd, J=10.7, 7.8 Hz, 1H), 2.54 (q, J=8.8 Hz, 1H), 2.36 (s, 3H), 2.33-2.24 (m, 1H), 2.22 (s, 3H), 2.13-2.04 (m, 1H), 1.45 (dd, J=6.9, 1.1 Hz, 3H). LC-MS: m/z=618.20 [M+H]+; 616.16 [M−H]−.

### Example 102 and 103: 5-((1S,2R)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one and 5-((1S,2S)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

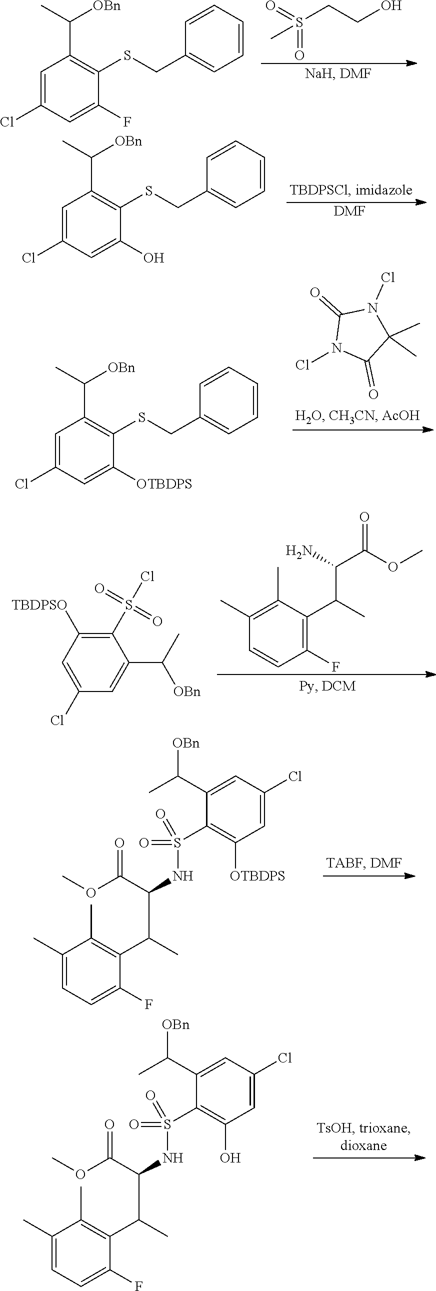

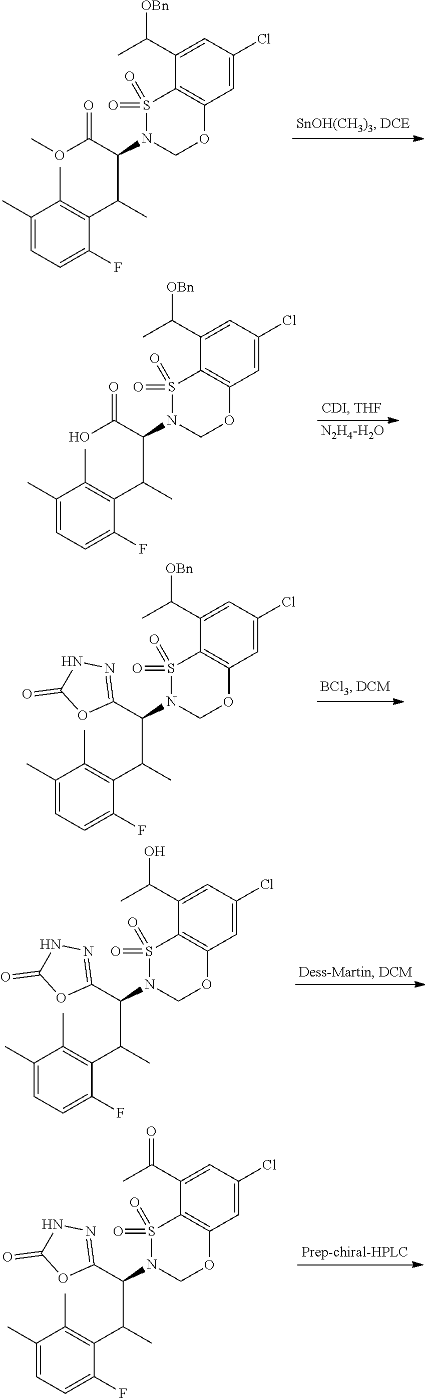

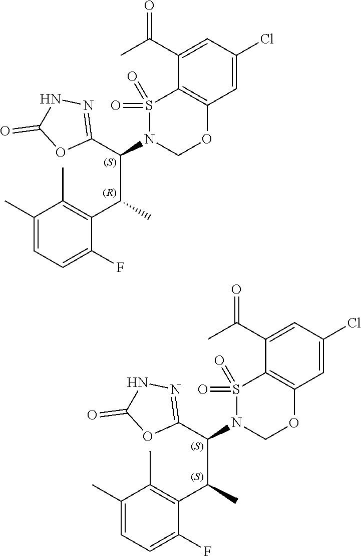

### Step 1: Synthesis of 3-(1-(benzyloxy)ethyl)-2-(benzylthio)-5-chlorophenol

Into a 100 mL round-bottom flask were added 1-[1-(benzyloxy)ethyl]-2-(benzylsulfanyl)-5-chloro-3-fluorobenzene (see ex.68, step 3) (1.6 g, 4.135 mmol, 1 equiv), DMF (16 mL) and 2-methanesulfonylethanol (1.03 g, 8.270 mmol, 2 equiv) at room temperature. To the above mixture was added NaH (827.00 mg, 20.675 mmol, 5 equiv, 60%) in portions at room temperature. The resulting mixture was stirred for additional 1 h at 80° C. The resulting mixture was quenched with H₂O (20 ml), and extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 100% to 100% gradient in 10 min; detector, UV 254 nm. This resulted in 3-[1-(benzyloxy)ethyl]-2-(benzylsulfanyl)-5-chlorophenol (1.07 g, 67.2%) as a yellow oil.

### Step 2: Synthesis of (3-(1-(benzyloxy)ethyl)-2-(benzylthio)-5-chlorophenoxy)(tert-butyl)diphenylsilane

Into a 100 mL round-bottom flask were added 3-[1-(benzyloxy)ethyl]-2-(benzylsulfanyl)-5-chlorophenol (2.5 g, 6.495 mmol, 1 equiv), DMF (25 mL), TBDPSCl (5.07 mL, 19.485 mmol, 3 equiv) and imidazole (6.63 g, 97.425 mmol, 15 equiv) at room temperature. The resulting mixture was stirred for 1 h at 100° C. under air atmosphere. The resulted solution was purified by reversed-phase flash chromatography with the following conditions: column, C18 silica gel; mobile phase, MeCN in Water (0.1% FA), 10% to 50% gradient in 10 min; detector, UV 254 nm. This resulted in 3-[1-(benzyloxy)ethyl]-2-(benzylsulfanyl)-5-chlorophenoxy(tert-butyl)diphenylsilane (2.5 g, 61.8%) as a colorless oil.

### Step 3: Synthesis of 2-(1-(benzyloxy)ethyl)-6-((tert-butyldiphenylsilyl)oxy)-4-chlorobenzenesulfonyl chloride

Into a 100 mL round-bottom flask were added 3-[1-(benzyloxy)ethyl]-2-(benzylsulfanyl)-5-chlorophenoxy(tert-butyl)diphenylsilane (2.5 g, 4.011 mmol, 1 equiv), ACN (16 mL), AcOH (1.15 mL, 20.055 mmol, 5 equiv) and H₂O (0.65 mL, 36.099 mmol, 9 equiv) at room temperature. To the above mixture was added 1,3-dichloro-5,5-dimethylimidazolidine-2,4-dione (1.58 g, 8.022 mmol, 2 equiv) in portions at 0° C. The resulting mixture was stirred for additional 0.5 h at 0° C. The residue was purified by silica gel column chromatography, eluted with PE/EA (3:1) to afford 2-[1-(benzyloxy)ethyl]-6-[(tert-butyldiphenylsilyl)oxy]-4-chlorobenzenesulfonyl chloride (2.2 g, 91.5%) as a yellow oil.

### Step 4: Synthesis of methyl (2S)-2-((2-(1-(benzyloxy)ethyl)-6-((tert-butyldiphenylsilyl)oxy)-4-chlorophenyl)sulfonamido)-3-(6-fluoro-2,3-dimethylphenyl)butanoate

Into a 100 mL round-bottom flask were added methyl (2S)-2-amino-3-(6-fluoro-2,3-dimethylphenyl)butanoate (0.65 g, 2.716 mmol, 1.00 equiv), DCM (10 mL) and pyridine (1.77 mL, 21.930 mmol, 5 equiv). To the above mixture was added 2-[1-(benzyloxy)ethyl]-6-[(tert-butyldiphenylsilyl)oxy]-4-chlorobenzenesulfonyl chloride (2.63 g, 4.386 mmol, 1.00 equiv) in DCM (10 mL) dropwise at 0° C. The resulting mixture was stirred overnight at room temperature. The residue was purified by silica gel column chromatography, eluted with PE/EA (3:1) to afford methyl (2S)-2-(2-[1-(benzyloxy)ethyl]-6-[(tert-butyldiphenylsilyl)oxy]-4-chlorobenzenesulfonamido-3-(6-fluoro-2,3-dimethylphenyl)butanoate (1.5 g, 68.81%) as a colorless oil.

### Step 5: synthesis of methyl (2S)-2-((2-(1-(benzyloxy)ethyl)-4-chloro-6-hydroxyphenyl)sulfonamido)-3-(6-fluoro-2,3-dimethylphenyl)butanoate

Into a 250 mL round-bottom flask were added methyl (2S)-2-(2-[1-(benzyloxy)ethyl]-6-[(tert-butyldiphenylsilyl)oxy]-4-chlorobenzenesulfonamido-3-(6-fluoro-2,3-dimethylphenyl)butanoate (2.2 g, 2.742 mmol, 1 equiv), THF (44.00 mL) and TBAF·3H₂O (1037.97 mg, 3.290 mmol, 1.2 equiv) at room temperature. The resulting mixture was stirred for 1 h at room temperature under air atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in methyl (2S)-2-(2-[1-(benzyloxy)ethyl]-4-chloro-6-hydroxybenzenesulfonamido-3-(6-fluoro-2,3-dimethylphenyl)butanoate (900 mg, 58.20%) as a colorless oil.

### Step 6: synthesis of methyl (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoate

Into a 40 mL sealed tube were added methyl (2S)-2-(2-[1-(benzyloxy)ethyl]-4-chloro-6-hydroxybenzenesulfonamido-3-(6-fluoro-2,3-dimethylphenyl)butanoate (949 mg, 1.682 mmol, 1 equiv), dioxane (10 mL), trioxane (2273.23 mg, 25.230 mmol, 15 equiv) and TsOH (289.71 mg, 1.682 mmol, 1 equiv) at room temperature. The resulting mixture was stirred overnight at 100° C. under air atmosphere. The resulting mixture was concentrated under reduced pressure. The residue was purified by reversed-phase flash chromatography with the following conditions: column, C18 silica gel; mobile phase, MeCN in Water (0.1% FA), 100% gradient in 10 min; detector, UV 220 nm. This resulted in synthesis of methyl (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoate (900 mg, 92.86%) as a white solid.

### Step 7: synthesis of (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoic acid

Into a 40 mL sealed tube were added methyl (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoate (516 mg, 0.896 mmol, 1 equiv), DCE (15.00 mL) and trimethyltin hydroxide (4.24 g, 23.430 mmol, 15 equiv) at room temperature. The resulting mixture was stirred overnight at 60° C. under air atmosphere. The resulting mixture was filtered, the filter cake was washed with CH₂Cl₂ (2×5 mL). The filtrate was concentrated under reduced pressure. The residue was purified by reversed-phase flash chromatography with the following conditions: column, C18 silica gel; mobile phase, MeCN in Water (0.1% FA), 80% to 85% gradient in 10 min; detector, UV 254 nm. This resulted in (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoic acid (690 mg, 78.58%) as a white solid.

### Step 8: synthesis of 5-((1S)-1-(8-(1-(benzyloxy)ethyl)-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

Into a 100 mL round-bottom flask were added (2S)-2-(8-(1-(benzyloxy)ethyl)-6-chloro-1,1-dioxidobenzo[e][1,4,3]oxathiazin-2 (3H)-yl)-3-(6-fluoro-2,3-dimethylphenyl)butanoic acid (439 mg, 0.781 mmol, 1 equiv), THF (10 mL) and CDI (1 g, 6.167 mmol, 7.90 equiv) at room temperature. The resulting mixture was stirred for 0.5 h at room temperature under air atmosphere. To the above mixture was added NH₂NH₂·H₂O (0.5 mL, 10.287 mmol, 13.17 equiv) in portions over 30 min at 0° C. The resulting mixture was stirred for additional 0.5 h at 0° C. The resulting mixture was extracted with EtOAc (2×50 mL), dried over anhydrous Na₂SO₄. After filtration, the filtrate was concentrated under reduced pressure. Into a 100 mL round-bottom flask were added the above crude product, dioxane (10 mL) and CDI (1 g, 6.167 mmol, 7.90 equiv) at room temperature. The resulting mixture was stirred for 1 h at room temperature under air atmosphere. The resulting mixture was concentrated under reduced pressure. This resulted in 5-((1S)-1-(8-(1-(benzyloxy)ethyl)-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (407 mg, 86.55%) as a yellow solid.

### Step 9: synthesis of 5-((1S)-1-(6-chloro-8-(1-hydroxyethyl)-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

Into a 100 mL round-bottom flask were added 5-((1S)-1-(8-(1-(benzyloxy)ethyl)-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (675 mg, 1.121 mmol, 1 equiv) and DCM (20 mL) at room temperature. To the above mixture was added BCl₃ (3.36 mL, 3.363 mmol, 3 equiv) dropwise at 0° C. The resulting mixture was stirred for additional 1 h at 0° C. The reaction was quenched by the addition of Water (50 mL) at 0° C. The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in of 5-((1S)-1-(6-chloro-8-(1-hydroxyethyl)-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (260 mg, 45.30%) as a white solid.

### Step 10: synthesis of 5-((1S,2R)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one and 5-((1S,2S)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one

Into a 100 mL round-bottom flask were added of 5-((1S)-1-(6-chloro-8-(1-hydroxyethyl)-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (260 mg, 0.508 mmol, 1 equiv), DCM (10.40 mL) and dess-martin reagent (430.81 mg, 1.016 mmol, 2 equiv) at room temperature. The resulting mixture was stirred for 0.5 h at room temperature under air atmosphere. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-((1S)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one and 5-((1S,2S)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (210 mg, 81.09/6) as a mixture of diastereoisomers.

The diastereoisomers were separated by Prep-Chiral-HPLC with the following conditions: Column: Column: Lux Sum Cellulose-4, 2.12*25 cm, 5 μm; Mobile Phase A: Hex (0.1% FA)-HPLC, Mobile Phase B: EtOH-HPLC; Flow rate: 20 mL/min; Gradient: isocratic 20; Wave Length: 254/220 nm; RT1 (min): 13.408; RT2 (min): 18.047; Sample Solvent: EtOH-HPLC; Injection Volume: 2.0 mL; Number Of Runs: 16. This resulted in:

102: 5-((1S,2R)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (45.7 mg). LCMS:(ES,m/z): [M+H]+=509.95. ¹H NMR (400 MHz, Methanol-d₄) δ 7.42 (d, J=2.1 Hz, 1H), 7.09 (d, J=2.0 Hz, 1H), 7.02 (dd, J=8.4, 5.7 Hz, 1H), 6.74 (dd, J=12.2, 8.4 Hz, 1H), 6.06 (d, J=12.9 Hz, 1H), 5.94 (d, J=13.0 Hz, 1H), 5.54 (d, J=11.9 Hz, 1H), 3.96 (dt, J=12.4, 6.7 Hz, 1H), 2.67 (s, 3H), 2.39 (s, 3H), 2.25 (s, 3H), 1.48-1.42 (m, 3H).

103: 5-((1S,2S)-1-(8-acetyl-6-chloro-1,1-dioxidobenzo[e][1,4,3]oxathiazin-2 (3H)-yl)-2-(6-fluoro-2,3-dimethylphenyl)propyl)-1,3,4-oxadiazol-2 (3H)-one (50.8 mg). LCMS:(ES,m/z): [M+H]+=509.95. ¹H NMR (400 MHz, Methanol-d₄) δ 7.36 (dd, J=45.5, 2.0 Hz, 1H), 7.12-6.99 (m, 2H), 6.76 (td, J=11.9, 8.4 Hz, 1H), 5.75 (d, J=12.8 Hz, 1H), 5.65 (d, J=12.8 Hz, 1H), 5.58-5.47 (m, 1H), 4.14-3.86 (m, 1H), 2.64 (d, J=25.9 Hz, 3H), 2.37 (d, J=25.5 Hz, 3H), 2.26 (d, J=9.4 Hz, 3H), 1.45 (dd, J=7.0, 1.1 Hz, 1H), 1.31 (d, J=6.8 Hz, 2H), 1.20 (t, J=7.1 Hz, 3H).

### Example 104: 5-((1S,2R)-1-(6-chloro-4-((R)-1-(2-fluoroethyl)pyrrolidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following procedure used for example 30. For Step 1, 2-methoxypropylamine was substituted with (3R)-1-(2-fluoroethyl)pyrrolidin-3-amine which was synthesized using the following procedure: To a solution of (3R)-(Boc-amino)pyrrolidine (4.0 g, 21.476 mmol, 1.0 eq) and 1-fluoro-2-iodoethane (4.483 g, 25.771 mmol, 1.2 eq) in anhydrous DMF (40.0 mL) was added DIPEA (7.482 mL, 42.952 mmol, 2.0 eq). The reaction mixture was heated at 70° C. overnight. Reaction progress was monitored by TLC (5% MeOH in DCM). After complete consumption of starting material, the reaction mixture was cooled to room temperature, diluted with water, and extracted with ethyl acetate. Organic layers were combined, washed with brine, dried over sodium sulphate, and concentrated under reduced pressure.

The product was purified by FCC (3% MeOH in DCM) to afford tert-butyl N-[(3R)-1-(2-fluoroethyl)pyrrolidin-3-yl]carbamate (4.0 g, 15.50 mmol, yield: 72%).

¹H NMR (400 MHz, Methanol-d₄) δ7.59 (d, J=8.5 Hz, 1H), 7.09 (d, J=1.9 Hz, 1H), 7.00 (dd, J=8.5, 5.7 Hz, 1H), 6.83 (dd, J=8.4, 1.8 Hz, 1H), 6.74 (dd, J=12.1, 8.4 Hz, 1H), 5.48 (dd, J=11.9, 2.0 Hz, 1H), 5.27 (d, J=1.4 Hz, 2H), 4.73 (t, J=4.8 Hz, 1H), 4.61 (t, J=4.8 Hz, 1H), 4.45-4.37 (m, 1H), 3.92 (dq, J=13.4, 6.9 Hz, 1H), 3.41 (dd, J=11.4, 2.3 Hz, 1H), 3.22-3.12 (m, 1H), 2.99 (ddt, J=29.1, 13.9, 4.8 Hz, 1H), 2.80 (ddt, J=27.7, 13.9, 4.8 Hz, 1H), 2.60 (dd, J=11.2, 7.4 Hz, 1H), 2.45-2.39 (m, 1H), 2.38 (s, 3H), 2.36-2.26 (m, 1H), 2.23 (s, 3H), 1.86-1.75 (m, 1H), 1.45 (dd, J=7.0, 1.1 Hz, 3H). LC-MS: m/z=582.21 [M+H]+.

### Example 105: 5-((1S,2R)-1-(6-chloro-4-(2-(4-methylpiperazin-1-yl)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 30. For Step 1, 2-methoxypropylamine was substituted with 2-(4-methyl-piperazin-1-yl)-ethylamine.

¹H NMR (400 MHz, Methanol-d₄) δ7.57 (d, J=8.3 Hz, 1H), 6.99 (dd, J=8.4, 5.7 Hz, 1H), 6.81-6.76 (m, 2H), 6.71 (dd, J=12.1, 8.4 Hz, 1H), 5.51 (d, J=14.5 Hz, 1H), 5.41 (dd, J=11.9, 1.6 Hz, 1H), 5.10 (d, J=14.6 Hz, 1H), 3.92 (dq, J=13.5, 6.9 Hz, 1H), 3.60-3.50 (m, 1H), 3.36-3.27 (m, 1H), 2.79-2.41 (m, 10H), 2.38 (s, 3H), 2.35 (s, 3H), 2.22 (s, 3H), 1.46 (dd, J=6.9, 1.1 Hz, 3H). LC-MS: m/z=593.17 [M+H]+; 591.18 [M−H]−.

### Example 106: 5-((1S,2R)-1-(6-chloro-4-((S)-1-(2-fluoroethyl)pyrrolidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 105. using (3S)-1-(2-fluoroethyl)pyrrolidin-3-amine. The fluoro displacement was carried out at 95° C. for 3 days. Step 2 was carried out at 70° C. for 1 day. ¹H NMR (400 MHz, Methanol-d₄) δ 7.56 (d, J=8.4 Hz, 1H), 7.23 (d, J=1.8 Hz, 1H), 7.00 (dd, J=8.4, 5.7 Hz, 1H), 6.79 (dd, J=8.4, 1.8 Hz, 1H), 6.73 (dd, J=12.1, 8.4 Hz, 1H), 5.46-5.38 (m, 2H), 5.32 (d, J=14.4 Hz, 1H), 4.66 (dd, J=5.7, 4.1 Hz, 1H), 4.54 (dd, J=5.7, 4.2 Hz, 1H), 4.35-4.28 (m, 1H), 4.00-3.90 (m, 1H), 3.25-3.19 (m, 1H), 3.10 (dd, J=10.7, 2.9 Hz, 1H), 2.99-2.89 (m, 1H), 2.85 (dt, J=16.5, 4.9 Hz, 1H), 2.75 (tt, J=13.9, 4.4 Hz, 1H), 2.54 (dd, J=10.8, 7.7 Hz, 1H), 2.44-2.38 (m, 1H), 2.37 (s, 3H), 2.32-2.23 (m, 1H), 2.22 (s, 3H), 2.07 (ddd, J=13.8, 9.2, 5.3 Hz, 1H), 1.45 (dd, J=6.9, 1.1 Hz, 3H). LC-MS: m/z=582.24 [M+H]+; 580.16 [M−H]−.

### Example 107: 5-((1S,2R)-1-(6-chloro-4-(1-(2,2-difluoroethyl)azetidin-3-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following procedure described for example 96 substituting 2,2,2-trifluoroethyl trifluoromethanesulfonate with 2,2-difluoroethyl trifluoromethanesulfonate. ¹H NMR (400 MHz, Methanol-d₄) δ 7.61 (d, J=8.4 Hz, 1H), 7.00 (dd, J=8.4, 5.7 Hz, 1H), 6.87 (dd, J=8.4, 1.8 Hz, 1H), 6.73 (dd, J=12.1, 8.4 Hz, 1H), 6.59 (d, J=1.9 Hz, 1H), 5.89 (tt, J=55.7, 4.1 Hz, 1H), 5.44 (dd, J=11.8, 1.7 Hz, 1H), 5.34 (d, J=14.4 Hz, 1H), 5.19 (d, J=14.4 Hz, 1H), 4.19 (p, J=7.0 Hz, 1H), 3.99-3.90 (m, 1H), 3.90-3.80 (m, 2H), 3.58 (t, J=7.4 Hz, 1H), 3.27 (t, J=7.3 Hz, 1H), 2.96 (td, J=15.6, 4.1 Hz, 2H), 2.38 (s, 3H), 2.23 (s, 3H), 1.47 (dd, J=6.9, 1.1 Hz, 3H). ¹⁹F NMR (376 MHz, Methanol-d₄) δ −118.04 (dd, J=12.1, 5.6 Hz), −122.96 (dt, J=55.8, 15.6 Hz). LC-MS: m/z=586.15 [M+H]⁺; 583.92 [M−H]−.

### Example 108: 5-((1S,2R)-1-(6-chloro-4-(2-fluoroethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a cooled solution 5-((1S,2R)-1-(6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (see ex.50) (1.0 eq) in anhydrous DCM (1.2 mL) was added diethylaminosulfur trifluoride (DAST) (2.0 eq) at 0° C. and stirred for 1 h until the reaction was completed. The reaction mixture was poured into saturated NaHCO₃ aqueous solution and extracted with DCM. Combined organic layers were dried over Na₂SO₄ and concentrated under vacuum. The title compound was purified by pTLC (10 mg, yield: 21%). ¹H NMR (400 MHz, Methanol-d₄) δ 7.62-7.58 (m, 1H), 6.99 (dd, J=8.4, 5.7 Hz, 1H), 6.86-6.80 (m, 2H), 6.72 (dd, J=12.1, 8.4 Hz, 1H), 5.49-5.45 (m, 1H), 5.45-5.42 (m, 1H), 5.13 (d, J=14.8 Hz, 1H), 4.69-4.65 (m, 1H), 4.57-4.53 (m, 1H), 3.97-3.88 (m, 1H), 3.86-3.71 (m, 1H), 3.60-3.47 (m, 1H), 2.38 (s, 3H), 2.22 (s, 3H), 1.45 (dd, J=6.9, 1.1 Hz, 3H). LC-MS: m/z=510.86 [M−H]−.

### Example 109: 5-((1S,2R)-1-(8-acetyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

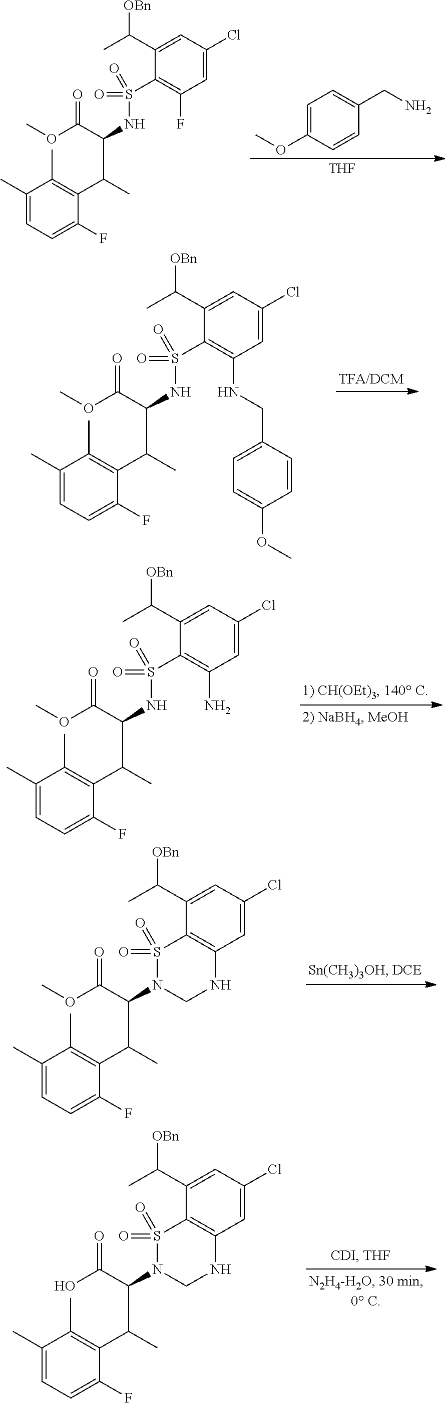

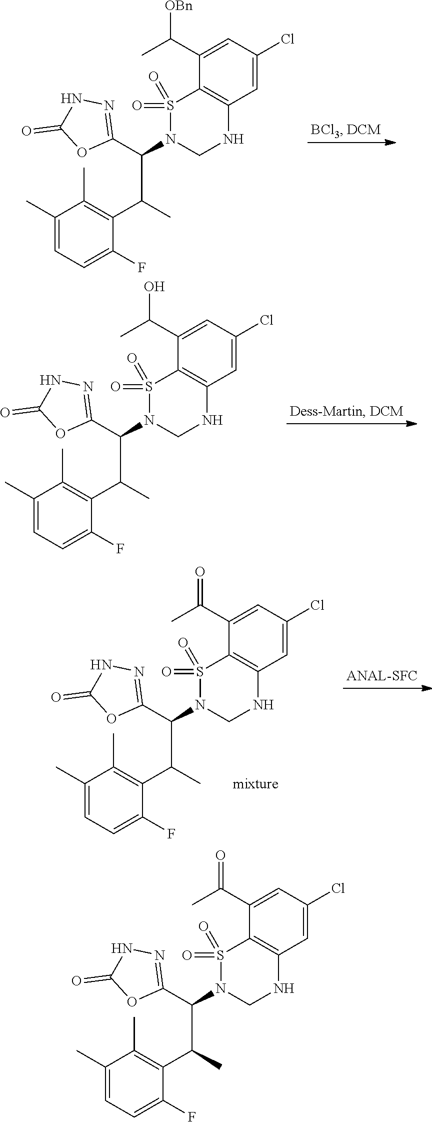

### Step 1: Synthesis of methyl (2S)-2-(2-[1-(benzyloxy)ethyl]-4-chloro-6-([(4-methoxyphenyl)methyl]aminobenzenesulfonamido-3-(6-fluoro-2,3-dimethylphenyl)butanoate

To a stirred solution of methyl (2S)-2-(2-[1-(benzyloxy)ethyl]-4-chloro-6-fluorobenzenesulfonamido-3-(6-fluoro-2,3-dimethylphenyl)butanoate (see ex. 68) (1.47 g, 2.597 mmol, 1 equiv) in THF (29.40 mL) was added (4-methoxyphenyl)methanamine (3.39 mL, 25.970 mmol, 10 equiv) at room temperature. The resulting mixture was stirred for 3 days at 65° C. The resulted solution was evaporated directly. The residue was purified by reversed-phase flash chromatography with the following conditions: column, C18 silica gel; mobile phase, MeCN in Water (0.1% FA), 10% to 100% gradient in 15 min; detector, UV 254 nm. This resulted in methyl (2S)-2-(2-[1-(benzyloxy)ethyl]-4-chloro-6-([(4-methoxyphenyl)methyl]aminobenzenesulfonamido-3-(6-fluoro-2,3-dimethylphenyl)butanoate (1.4 g, 78.91%) as a white solid.

### Step 2: Synthesis of methyl (2S)-2-(2-amino-6-[1-(benzyloxy)ethyl]-4-chlorobenzenesulfonamido-3-(6-fluoro-2,3-dimethylphenyl)butanoate

To a stirred solution of methyl (2S)-2-(2-[1-(benzyloxy)ethyl]-4-chloro-6-([(4-methoxyphenyl)methyl]aminobenzenesulfonamido-3-(6-fluoro-2,3-dimethylphenyl)butanoate (1.4 g, 2.049 mmol, 1 equiv) in DCM (10 mL) was added TFA (10 mL) dropwise at room temperature. The resulting mixture was stirred for 30 min at room temperature. The resulting mixture was concentrated under vacuum. The residue was purified by reversed-phase flash chromatography with the following conditions: column, C18 silica gel; mobile phase, MeCN in Water (0.1% FA), 30% to 100% gradient in 10 min; detector, UV 254 nm. This resulted in methyl (2S)-2-(2-amino-6-[1-(benzyloxy)ethyl]-4-chlorobenzenesulfonamido-3-(6-fluoro-2,3-dimethylphenyl)butanoate (1.09 g, 94.47%) as a white solid.

### Step 3: Synthesis of methyl (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoate

To a stirred solution of methyl (2S)-2-(2-amino-6-[1-(benzyloxy)ethyl]-4-chlorobenzenesulfonamido-3-(6-fluoro-2,3-dimethylphenyl)butanoate (1.09 g, 1.936 mmol, 1 equiv) was added triethyl orthoformate (25 mL, 0.007 mmol, 0.19 equiv) dropwise at room temperature. The resulting mixture was stirred overnight at 140° C. The resulting mixture was concentrated under vacuum. The resulting mixture was diluted with MeOH (5 mL). To the above mixture was added NaBH₄ (146.46 mg, 3.872 mmol, 2 equiv) at room temperature. The resulting mixture was stirred for additional 30 min at room temperature. The reaction was quenched with water (50 ml) at room temperature. The resulting mixture was extracted with EtOAc (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 100% gradient in 10 min; detector, UV 254 nm. This resulted in methyl (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoate (870 mg, 78.15%) as a brown solid.

### Step 4: Synthesis of (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoic acid

To a stirred solution of methyl (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoate (9.1 mg, 0.016 mmol, 1 equiv) in DCE (10 mL, 126.3 mmol, 84 equiv) was added trimethyltin hydroxide (4.08 g, 22.7 mmol, 15 equiv) in portions at room temperature. The resulting mixture was stirred for 3 days at 65° C. The resulting mixture was concentrated under vacuum. The residue was purified by reversed-phase flash chromatography with the following conditions: column, C18 silica gel; mobile phase, MeCN in Water (0.1% FA), 10% to 100% gradient in 15 min; detector, UV 254 nm. This resulted in Synthesis of (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoic acid (690 mg, 81.3%) as a white solid.

### Step 5: Synthesis of 5-((1S)-1-(8-(1-(benzyloxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution of (2S)-2-(8-(1-(benzyloxy)ethyl)-6-chloro-1,1-dioxido-3,4-dihydro-2H-benzo[e][1,2,4]thiadiazin-2-yl)-3-(6-fluoro-2,3-dimethylphenyl)butanoic acid (690 mg, 1.23 mmol, 1 equiv) in THF (10 mL was added CDI (797.7 mg, 4.92 mmol, 4 equiv) in portions at room temperature. The resulting mixture was stirred for 1 h at room temperature. To the above mixture was added NH₂NH₂·H₂O (358.7 μL, 7.38 mmol, 6 equiv) dropwise at 0° C. The resulting mixture was stirred for an additional 1 h at 0° C. The reaction was quenched with water (10 ml) at room temperature. The resulting mixture was extracted with EtOAc (3×10 mL). The combined organic layers were washed with brine (2×20 mL), dried over anhydrous Na₂SO₄. After filtration, the filtrate was concentrated under reduced pressure to afford intermediate as a colorless oil. To the above mixture was added CDI (797.7 mg, 4.92 mmol, 4 equiv) in portions at room temperature. The resulting mixture was stirred overnight at room temperature. The resulted solution was evaporated under vacuum and purified by reversed-phase flash chromatography with the following conditions: column, C18 silica gel; mobile phase, MeCN in Water (0.1% FA), 10% to 100% gradient in 10 min; detector, UV 254 nm. This resulted in 5-((1S)-1-(8-(1-(benzyloxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (350 mg, 47.4%) as a white solid.

### Step 6: Synthesis of 5-((1S)-1-(6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution 5-((1S)-1-(8-(1-(benzyloxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (350 mg, 0.58 mmol, 1 equiv) in DCM (5 mL) was added boron trichloride (2329 μL, 2.33 mmol, 4 equiv) dropwise at 0° C. under nitrogen atmosphere. The resulting mixture was stirred for 10 min at 0° C. under nitrogen atmosphere. The resulting mixture was concentrated under vacuum. The residue was purified by reversed-phase flash chromatography with the following conditions: column, C18 silica gel; mobile phase, MeCN in Water (0.1% FA), 10% to 100% gradient in 10 min; detector, UV 254 nm. This resulted in 5-((1S)-1-(6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (275 mg, 92.4%) as a white solid.

### Step 7: Synthesis of 5-((1S,2R)-1-(8-acetyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a stirred solution of 5-((1S)-1-(6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (210 mg, 0.039 mmol, 1 equiv) in DCM (3 mL) was added Dess-Martin reagent (350 mg, 0.078 mmol, 2 equiv) in portions at room temperature. The resulting mixture was stirred for 30 min at room temperature. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 100% gradient in 10 min; detector, UV 254 nm. The resulting mixture was concentrated under vacuum

The residue was purified by Prep-Chiral-SFC(Column: CHIRAL ART Cellulose-SB 3*25 cm, Sum; Mobile Phase A: CO2, Mobile Phase B: MeOH (1%-2M-NH₃-MeOH); Flow rate: 85 mL/min; Gradient: isocratic 30% B; Column Temperature (° C.): 35; Back Pressure (bar): 100; Wave Length: 220 nm; RT1 (min): 3.22; RT2 (min): 3.93; Sample Solvent: MEOH; Injection Volume: 2 mL). This resulted in Synthesis of 5-((1S,2R)-1-(8-acetyl-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (30 mg, 37%). LCMS:(ES,m/z) (MS+1)=509. ¹H NMR (400 MHz, Methanol-d₄) δ 7.00-6.96 (dd, J=8.4, 5.7 Hz, 1H), 6.79-6.78 (d, J=2.0 Hz, 1H), 6.76-6.68 (m, 2H), 5.36-5.30 (dd, J=11.8, 1.6 Hz, 1H), 5.27-5.23 (d, J=14.8 Hz, 1H), 5.17-5.05 (d, J=14.8 Hz, 1H), 3.90-3.82 (dqd, J=13.7, 7.0, 1.4 Hz, 1H), 2.60 (s, 3H), 2.43 (d, J=15.1 Hz, 1H), 2.27 (s, 3H), 2.24 (s, 3H), 1.46 (dd, J=7.0, 1.1 Hz, 3H).

### Example 110: 5-((1S,2R)-1-(8-acetyl-6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

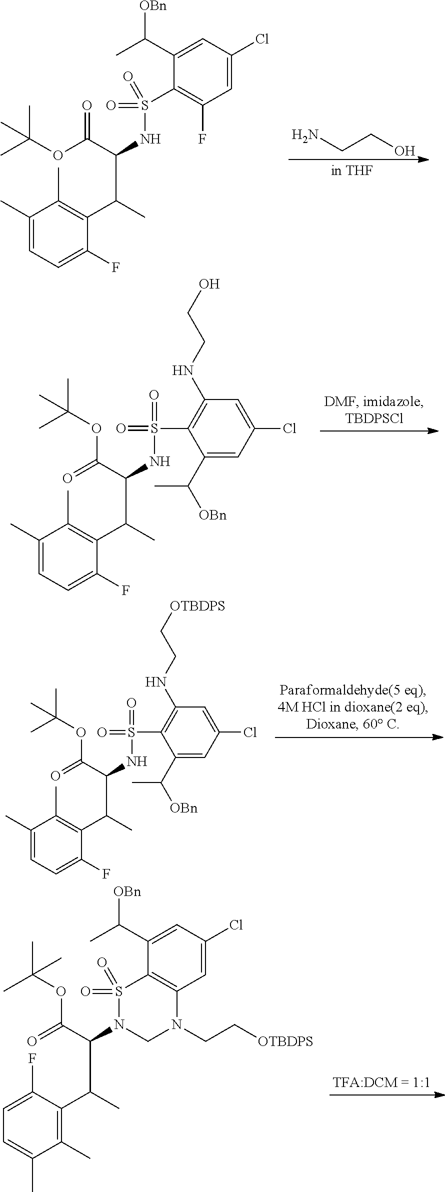

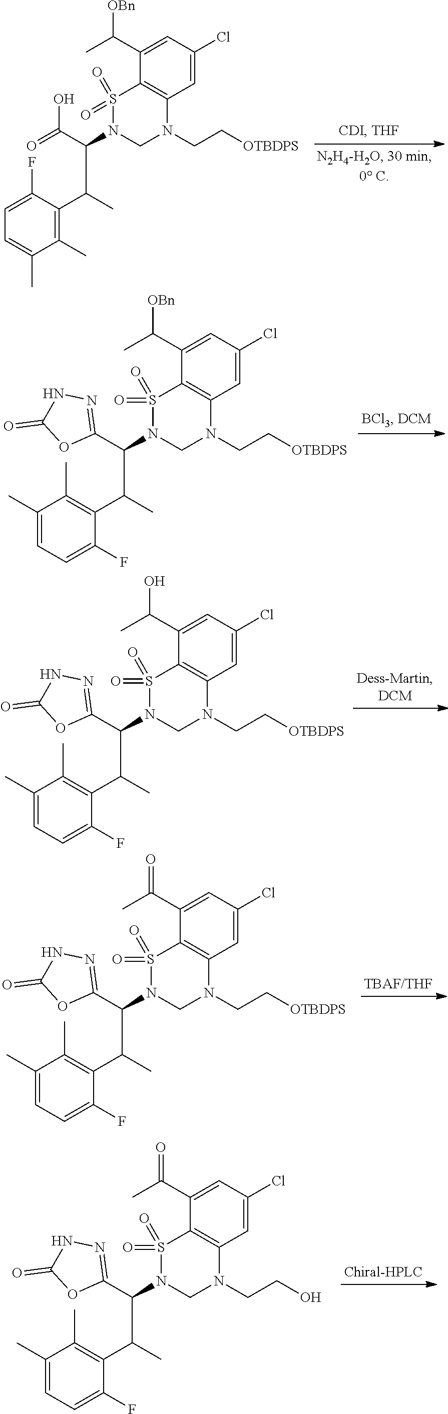

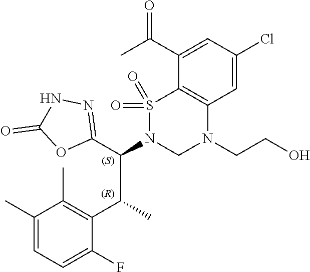

### Step 1: Synthesis of tert-butyl (2S)-2-(2-[1-(benzyloxy)ethyl]-4-chloro-6-[(2-hydroxyethyl)amino]benzenesulfonamido-3-(6-fluoro-2,3-dimethylphenyl)butanoate

Into a 40 mL sealed tube were added tert-butyl (2S)-2-(2-[1-(benzyloxy)ethyl]-4-chloro-6-fluorobenzenesulfonamido-3-(6-fluoro-2,3-dimethylphenyl)butanoate (see ex.68) (500 mg, 0.822 mmol, 1 equiv), DMSO (10 mL), TEA (571 μL, 4.1 mmol, 5 equiv) and ethanolamine (753.3 mg, 12.3 mmol, 15 equiv) at room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in tert-butyl (2S)-2-(2-[1-(benzyloxy)ethyl]-4-chloro-6-[(2-hydroxyethyl)amino]benzenesulfonamido-3-(6-fluoro-2,3-dimethylphenyl)butanoate (510 mg, 95.6%) as a white solid.

### Step 2: Synthesis of tert-butyl (2S)-2-(2-[1-(benzyloxy)ethyl]-6-((2-[(tert-butyldiphenylsilyl)oxy]ethylamino)-4-chlorobenzenesulfonamido-3-(6-fluoro-2,3-dimethylphenyl)butanoate

Into a 250 mL round-bottom flask were added tert-butyl (2S)-2-(2-[1-(benzyloxy)ethyl]-4-chloro-6-[(2-hydroxyethyl)amino]benzenesulfonamido-3-(6-fluoro-2,3-dimethylphenyl)butanoate (560 mg, 0.86 mmol, 1 equiv), DMF (11.2 mL), imidazole (880.9 mg, 12.9 mmol, 15 equiv) and TBDPSCl (672.9 μL, 2.59 mmol, 3 equiv) at room temperature. The resulting mixture was stirred for 1 h at room temperature under air atmosphere. The residue was purified by reversed-phase flash chromatography with the following conditions: column, C18 silica gel; mobile phase, MeCN in Water (0.1% FA), 100% gradient in 10 min; detector, UV 254 nm. This resulted in tert-butyl (2S)-2-(2-[1-(benzyloxy)ethyl]-6-((2-[(tert-butyldiphenylsilyl)oxy]ethylamino)-4-chlorobenzenesulfonamido-3-(6-fluoro-2,3-dimethylphenyl)butanoate (730 mg, 95.4%) as a white solid.

### Step 3: Synthesis tert-butyl (2S)-2-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl-3-(6-fluoro-2,3-dimethylphenyl)butanoate

Into a 40 mL sealed tube were added tert-butyl (2S)-2-(2-[1-(benzyloxy)ethyl]-6-((2-[(tert-butyldiphenylsilyl)oxy]ethylamino)-4-chlorobenzenesulfonamido-3-(6-fluoro-2,3-dimethylphenyl)butanoate (730 mg, 0.82 mmol, 1 equiv), dioxane (14 mL), Paraformaldehyde (370 mg, 4.11 mmol, 5 equiv) and HCl (411 μL, 1.64 mmol, 2 equiv) at room temperature. The resulting mixture was stirred overnight at 60° C. under air atmosphere. The resulting mixture was filtered, the filter cake was washed with EtOAc (3×5 mL).

The filtrate was concentrated under reduced pressure. The residue was purified by reversed-phase flash chromatography with the following conditions: column, C18 silica gel; mobile phase, MeCN in Water (0.1% FA), 100% gradient in 10 min; detector, UV 254 nm. This resulted in tert-butyl (2S)-2-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-3-(6-fluoro-2,3-dimethylphenyl)butanoate (690 mg, 99.5%) as a white solid.

### Step 4: Synthesis of (2S)-2-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-3-(6-fluoro-2,3-dimethylphenyl)butanoic acid

Into a 250 mL round-bottom flask were added tert-butyl (2S)-2-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-3-(6-fluoro-2,3-dimethylphenyl)butanoate (650 mg, 0.72 mmol, 1 equiv), DCM (9 mL) and TFA (3 mL) at room temperature. The resulting mixture was stirred for 0.5 h at room temperature under air atmosphere. The resulting mixture was concentrated under reduced pressure. The crude product was used in the next step directly without further purification.

### Step 5: Synthesis of 5-((1S)-1-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl-2-(6-fluoro-2,3-dimethylphenyl)propyl)-1,3,4-oxadiazol-2 (3H)-one

Into a 100 mL round-bottom flask were added 2S)-2-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-3-(6-fluoro-2,3-dimethylphenyl)butanoic acid (700 mg, 0.83 mmol, 1 equiv), THF (14 mL) and CDI (538 mg, 3.32 mmol, 4 equiv) at room temperature. The resulting mixture was stirred for 0.5 h at room temperature under air atmosphere. To the above mixture was added NH₂NH₂·H₂O (0.5 mL, 10.9 mmol, 13 equiv) in portions over 30 min at 0° C. The resulting mixture was stirred for additional 0.5 h at 0° C. The resulting mixture was extracted with EtOAc (2×8 mL), dried over anhydrous Na₂SO₄. After filtration, the filtrate was concentrated under reduced pressure. Into a 100 mL round-bottom flask were added to the above crude product, dioxane (14 mL) and CDI (538 mg, 3.32 mmol, 4 equiv) at room temperature. The resulting mixture was stirred for 0.5 h at room temperature under air atmosphere. The resulting mixture was concentrated under reduced pressure. The residue was purified by reversed-phase flash chromatography with the following conditions: column, C18 silica gel; mobile phase, MeCN in Water (0.1% FA), 100% gradient in 10 min; detector, UV 254 nm. This resulted in 5-((1S)-1-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (600 mg, 81.8%) as a white solid.

### Step 6: Synthesis of 5-((1S)-1-(4-(2-((tert-butyldiphenylsilyl)oxy)ethyl)-6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 100 mL round-bottom flask were added 5-((1S)-1-(8-(1-(benzyloxy)ethyl)-4-(2-((tert-butyldiphenylsilyl)oxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (580 mg, 0.66 mmol, 1 equiv) and DCM (12 mL) at room temperature. To the above mixture was added BCl₃ (2.63 mL, 2.62 mmol, 4 equiv) dropwise at 0° C. The resulting mixture was stirred for additional 1 h at 0° C. The resulting mixture was diluted with DCM (20 mL). The mixture was neutralized to pH 7 with saturated NaHCO₃ (aq.). The resulting mixture was extracted with CH₂Cl₂ (2×200 mL). The combined organic layers were washed with brine (1×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 70% to 80% gradient in 10 min; detector, UV 254 nm. This resulted in of 5-((1S)-1-(4-(2-((tert-butyldiphenylsilyl)oxy)ethyl)-6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (263 mg, 50.5%) as a white solid.

### Step 7: Synthesis of 5-((1S)-1-(8-acetyl-4-(2-((tert-butyldiphenylsilyl)oxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 100 mL round-bottom flask were added of 5-((1S)-1-(4-(2-((tert-butyldiphenylsilyl)oxy)ethyl)-6-chloro-8-(1-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (250 mg, 0.32 mmol, 1 equiv), DCM (5 mL) and Dess Martin reagent (267.3 mg, 0.63 mmol, 2 equiv) at room temperature. The resulting mixture was stirred for 1 h at room temperature under air atmosphere. The resulting mixture was concentrated under reduced pressure. The residue was purified by reversed-phase flash chromatography with the following conditions: column, C18 silica gel; mobile phase, MeCN in Water (0.1% FA), 70% to 80% gradient in 10 min; detector, UV 254 nm. This resulted in 5-((1S)-1-(8-acetyl-4-(2-((tert-butyldiphenylsilyl)oxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (230 mg, 92.2%) as a white solid. Step 8: Synthesis of 5-((1S,2R)-1-(8-acetyl-6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

Into a 50 mL round-bottom flask were added 5-((1S)-1-(8-acetyl-4-(2-((tert-butyldiphenylsilyl)oxy)ethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (250 mg, 0.32 mmol, 1 equiv), THF (5 mL) and TBAF (123.9 mg, 0.474 mmol, 1.5 equiv) at room temperature. The residue was purified by reversed-phase flash chromatography with the following conditions: column, C18 silica gel; mobile phase, MeCN in Water (0.1% FA), 58% to 60% gradient in 10 min; detector, UV 254 nm. After evaporation, the residue was repurified by Chiral-SFC with the following Column: CHIRALPAK IH 2*25 cm, Sum; Mobile Phase A: CO₂, Mobile Phase B: MeOH (0.1% FA+1%/-2M-NH₃-MeOH); Flow rate: 55 mL/min; Gradient: isocratic 30% B; Column Temperature (° C.): 35; Back Pressure (bar): 100; Wave Length: 220 nm; RT1 (min): 2.5; RT2 (min): 5; Sample Solvent: MEOH; Injection Volume: 3 mL; Number Of Runs: 4. Only the S,R isomer was isolated, this resulted in 5-((1S,2R)-1-(8-acetyl-6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (13.2 mg, 8.15%). LCMS:(ES,m/z): [M+H]*=553.00. ¹H NMR (400 MHz, Methanol-d₄) δ 7.01 (dd, J=8.4, 5.7 Hz, 1H), 6.93 (d, J=1.9 Hz, 1H), 6.83 (d, J=1.8 Hz, 1H), 6.73 (dd, J=12.1, 8.4 Hz, 1H), 5.59 (d, J=14.8 Hz, 1H), 5.40 (dd, J=11.8, 1.7 Hz, 1H), 5.17 (d, J=14.9 Hz, 1H), 4.01-3.88 (m, 1H), 3.82-3.61 (m, 3H), 3.39-3.33 (m, 1H), 2.62 (s, 3H), 2.39 (s, 3H), 2.24 (s, 3H), 1.48-1.41 (m, 3H).

### Example 111: 5-((1S,2R)-1-(6-chloro-4-((1s,3R)-3-(dimethylamino)cyclobutyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for example 30. For Step 1, 2-methoxypropylamine was substituted with N1,N1-dimethylcyclobutane-1,3-diamine which was synthesized using the following procedure:

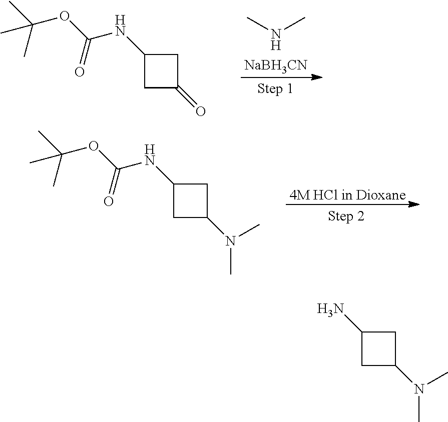

### Step 1. tert-butyl N-[3-(dimethylamino)cyclobutyl]carbamate

To a cooled mixture of tert-butyl N-(3-oxocyclobutyl)carbamate (2.0 g, 10.8 mmol, 1.0 eq) in anhydrous MeOH (54 mL, 0.2 M), 2M Dimethylamine in THF (16 ml), sodium cyanoborohydride (969 mg, 16.2 mmol, 1.5 eq) and acetic acid (3.1 mL) were added. The reaction was continued at rt overnight.

Reaction progress was monitored by TLC. Volatiles were removed in vacuo. Oily residue was diluted in AcOEt and washed thoroughly with sat. aq. NaHCO₃ solution. The layers were easily separated, dried and concentrated. The residue was purified by FCC to obtain tert-butyl N-[3-(dimethylamino)cyclobutyl]carbamate as a mixture of cis and trans isomers (1.68 g, yield: 65%).

### Step 2: N1,N1-dimethylcyclobutane-1,3-diamine

4M HC in 1,4-dioxane (9.5 mL, 38 mmol, 10.0 eq) was added to tert-butyl N-[3-(dimethylamino)cyclobutyl]carbamate (1.0 eq) and the mixture was stirred at rt overnight. Volatiles were removed in vacuo. The amine was obtained from its hydrochloride via neutralization with an ion exchange resin. The fluoro displacement with N1,N1-dimethylcyclobutane-1,3-diamine was carried out at 95° C. for 3 days. ¹H NMR (400 MHz, Methanol-d₄) δ7.59 (d, J=8.4 Hz, 1H), 6.99 (dd, J=8.4, 5.7 Hz, 1H), 6.84 (dd, J=8.4, 1.8 Hz, 1H), 6.76-6.68 (m, 2H), 5.41 (d, J=11.7 Hz, 1H), 5.29 (d, J=14.3 Hz, 1H), 5.23 (d, J=14.3 Hz, 1H), 3.92 (dq, J=12.0, 6.2, 5.5 Hz, 1H), 3.90-3.77 (m, 1H), 2.94 (p, J=7.8 Hz, 1H), 2.83-2.70 (m, 2H), 2.48 (s, 6H), 2.47-2.40 (m, 1H), 2.38 (s, 3H), 2.22 (s, 3H), 2.09 (q, J=9.7 Hz, 1H), 1.48 (dd, J=6.9, 1.1 Hz, 3H). LC-MS: m/z=564.21 [M+H]+; 561.88 [M−H]−.

### Example 112: 5-((1S,2R)-1-(6-chloro-4-((1s,3R)-3-hydroxycyclobutyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

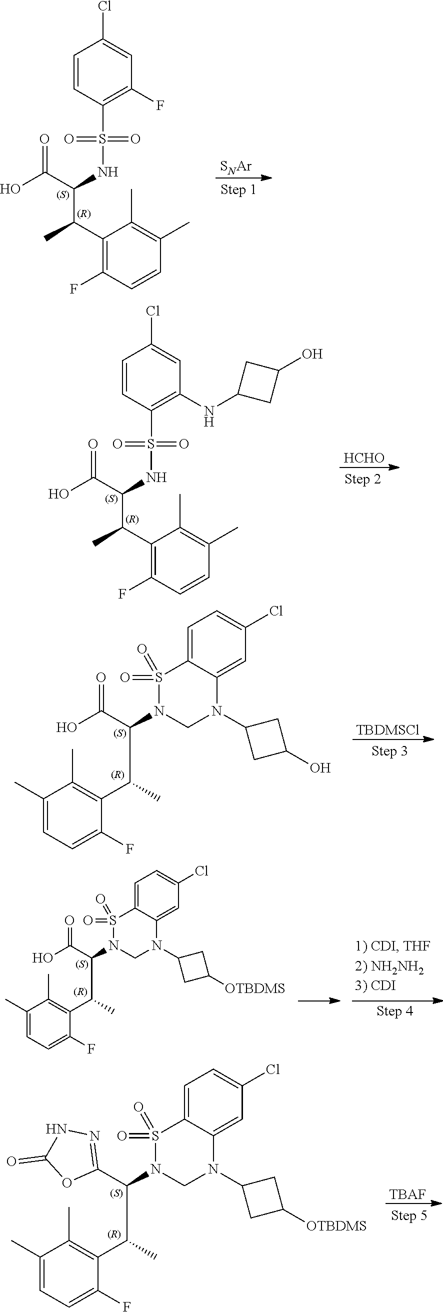

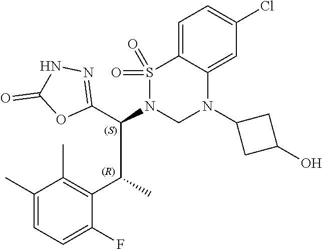

### Step 1. (2S,3R)-2-{4-chloro-2-[(3-hydroxycyclobutyl)amino]benzenesulfonamido}-3-(6-fluoro-2,3-dimethylphenyl)butanoic acid

The title compound was synthesized following the procedure described for (for example 30 substituting 2-(methoxypropyl)amine with 3-aminocyclobutanol. The reaction was carried out at 95° C. for 4 days.

### Step 2. (2S,3R)-2-(6-chloro-4-(3-hydroxycyclobutyl)-1,1-dioxido-3,4-dihydro-2H-benzo[e][1,2,4]thiadiazin-2-yl)-3-(6-fluoro-2,3-dimethylphenyl)butanoic acid

The title compound was synthesized following the procedure used in ex.45 0.62 5-((1S,2R)-1-(6-chloro-4-(2-methoxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one substituting (2S,3R)-2-{4-chloro-2-[(2-methoxyethyl)amino]benzenesulfonamido}-3-(6-fluoro-2,3-dimethylphenyl)butanoic acid with (2S,3R)-2-{4-chloro-2-[(3-hydroxycyclobutyl)amino]benzenesulfonamido}-3-(6-fluoro-2,3-dimethylphenyl)butanoic acid.

### Step 3. (2S,3R)-2-(4-(3-((tert-butyldimethylsilyl)oxy)cyclobutyl)-6-chloro-1,1-dioxido-3,4-dihydro-2H-benzo[e][1,2,4]thiadiazin-2-yl)-3-(6-fluoro-2,3-dimethylphenyl)butanoic acid

To a solution of (2S,3R)-2-(6-chloro-4-(3-hydroxycyclobutyl)-1,1-dioxido-3,4-dihydro-2H-benzo[e][1,2,4]thiadiazin-2-yl)-3-(6-fluoro-2,3-dimethylphenyl)butanoic acid (140 mg, 0.26 mmol, 1.0 eq) in anhydrous DCM (3.0 mL) were added TEA (0.075 mL, 0.535 mmol, 2.0 eq), DMAP (0.065 g, 0.535 mmol, 2 eq), and tert-Butyldimethylsilyl chloride (0.121 g, 0.803 mmol, 3.0 eq). The reaction was continued at rt for 3 days. The mixture was diluted with DCM, washed with 10% NaH2PO4, dried over Na₂SO₄, filtered and concentrated in vacuo. The product was purified by FCC (49 mg, yield: 25%).

### Step 4. 5-((1S,2R)-1-(4-(3-((tert-butyldimethylsilyl)oxy)cyclobutyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized according to procedure described for ex.45 substituting (2S,3R)-2-(6-chloro-4-(2-methoxyethyl)-1,1-dioxido-3,4-dihydro-2H-benzo[e][1,2,4]thiadiazin-2-yl)-3-(6-fluoro-2,3-dimethylphenyl)butanoic acid with (2S,3R)-2-(4-(3-((tert-butyldimethylsilyl)oxy)cyclobutyl)-6-chloro-1,1-dioxido-3,4-dihydro-2H-benzo[e][1,2,4]thiadiazin-2-yl)-3-(6-fluoro-2,3-dimethylphenyl)butanoic acid Step 5. 5-((1S,2R)-1-(6-chloro-4-((1s,3R)-3-hydroxycyclobutyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a cooled solution of 5-((1S,2R)-1-(4-(3-((tert-butyldimethylsilyl)oxy)cyclobutyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (42 mg, 0.05 mmol, 1.0 eq) in anhydrous THF (2 mL) at 0° C., 1M TBAF in THF was added (0.03 mL, 0.1 mmol, 2 eq). The reaction was continued at rt for 2 h. The mixture was diluted with DCM and washed with sat. NH₄C1 solution. Crude was purified by pHPLC to afford the title compound (10 mg, yield: 33%). ¹H NMR (400 MHz, Methanol-d₄) δ 7.58 (d, J=8.4 Hz, 1H), 6.99 (dd, J=8.4, 5.8 Hz, 1H), 6.81 (dd, J=8.4, 1.9 Hz, 1H), 6.75-6.68 (m, 2H), 5.40 (dd, J=11.9, 1.5 Hz, 1H), 5.29 (d, J=14.4 Hz, 1H), 5.20 (d, J=14.3 Hz, 1H), 4.09-3.99 (m, 1H), 3.90 (dq, J=13.4, 6.8 Hz, 1H), 3.58-3.49 (m, 1H), 2.88-2.76 (m, 2H), 2.38 (s, 3H), 2.36-2.28 (m, 1H), 2.22 (s, 3H), 1.97-1.88 (m, 1H), 1.47 (dd, J=6.9, 1.2 Hz, 3H). LC-MS: m/z=535.08 [M−H]−.

### Example 113: 5-((1S,2R)-1-(4-(2-aminoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

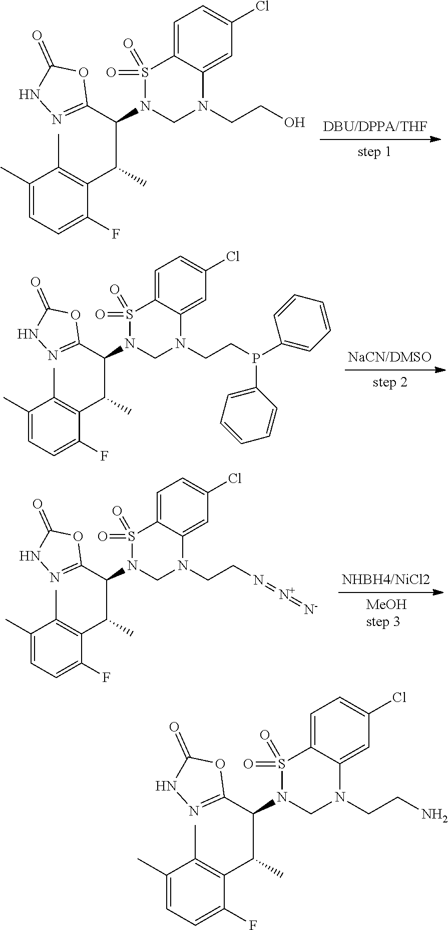

### Step 1: 5-((1S,2R)-1-(6-chloro-4-(2-(diphenylphosphaneyl)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a cooled solution of 5-((1S,2R)-1-(6-chloro-4-(2-hydroxyethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (see Ex 50) (0.185 g, 0.29 mmol, 1.0 eq) in anhydrous THF (3.7 mL), 1,8-Diazabicyclo[5.4.0]undec-7-ene (DBU, 0.097 g, 0.637 mmol, 2.2 eq) and DPPA (0.137 mL, 0.637 mmol, 2.2 eq) were added at 0° C. The reaction was continued for 2 h until starting material was consumed. The mixture was diluted with water and extracted with DCM. Aqueous phase was acidified to pH 5 and extracted with DCM again. Combined organic fractions were dried over Na₂SO₄ and concentrated under reduced pressure. The residue was purified by FCC to obtain 5-((1S,2R)-1-(6-chloro-4-(2-(diphenylphosphaneyl)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (182 mg, yield: 80%).

### Step 2: 5-((1S,2R)-1-(4-(2-azidoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a solution of 5-((1S,2R)-1-(6-chloro-4-(2-(diphenylphosphaneyl)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.182 g, 0.233 mmol, 1.0 eq) in anhydrous DMSO (3.64 mL) sodium azide (0.045 g, 0.698 mmol, 3.0 eq) was added and the reaction mixture was heated at 60° C. overnight. The mixture was diluted with AcOEt, repeatedly washed with 10% NaH₂PO₄ and concentrated under vacuum. Crude was purified by FCC to obtain 5-((1S,2R)-1-(4-(2-azidoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (120 mg, yield: 91%).

### Step 3: 5-((1S,2R)-1-(4-(2-aminoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

To a cooled solution of 5-((1S,2R)-1-(4-(2-azidoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.07 g, 0.124 mmol, 1.0 eq) in anhydrous MeOH (1.4 mL) were added anhydrous nickel(II) chloride (0.005 g, 0.037 mmol, 0.3 eq) and sodium borohydride (0.019 g, 0.496 mmol, 4 eq) portionwise. The reaction was continued at rt for 30 min. Upon completion, the mixture was poured into water and extracted with DCM. Organic fractions were dried over Na₂SO₄ and concentrated in vacuo. Crude was purified by pHPLC to afford 5-((1S,2R)-1-(4-(2-aminoethyl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (14 mg, yield: 22%). ¹H NMR (300 MHz, Methanol-d₄) δ 7.62 (d, J=8.9 Hz, 1H), 7.00 (dd, J=8.4, 5.8 Hz, 1H), 6.90-6.84 (m, 2H), 6.73 (dd, J=12.1, 8.4 Hz, 1H), 5.49 (d, J=14.5 Hz, 1H), 5.43 (d, J=12.1 Hz, 1H), 5.12 (d, J=14.5 Hz, 1H), 3.90 (dq, J=13.3, 6.9 Hz, 1H), 3.66 (dt, J=14.8, 7.1 Hz, 1H), 3.44 (dt, J=15.3, 7.4 Hz, 1H), 3.13-3.03 (m, 2H), 2.38 (s, 3H), 2.22 (s, 3H), 1.47 (dd, J=6.9, 1.1 Hz, 3H). LC-MS: m/z=510.13 [M+H]⁺; 508.13 [M−H]−.

### Example 114: 5-((1S,2R)-1-(4-(benzyloxy)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following the procedure used for Ex.30. For Step 1, 2-methoxypropylamine was substituted with O-benzylhydroxylamine. Step 1 was carried out at 105° C. for 3 days. ¹H NMR (400 MHz, Methanol-d₄) δ 7.62 (d, J=8.5 Hz, 1H), 7.53-7.39 (m, 5H), 7.07 (dd, J=8.5, 2.0 Hz, 1H), 7.04-6.97 (m, 2H), 6.74 (dd, J=12.1, 8.4 Hz, 1H), 5.58 (dd, J=11.6, 1.7 Hz, 1H), 5.24 (d, J=14.1 Hz, 1H), 5.20 (d, J=13.9 Hz, 1H), 5.02 (d, J=11.0 Hz, 1H), 4.88 (d, 1H), 3.91-3.85 (m, 1H), 2.38 (s, 3H), 2.24 (s, 3H), 1.41 (dd, J=7.0, 1.1 Hz, 3H). LC-MS: m/z=571.08 [M−H]

### Example 115: 5-((1S,2R)-1-(6-chloro-4-(2-(methylamino)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

tert-butyl (2-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)ethyl)(methyl)carbamate was synthesized following the procedure used for example 30. For Step 1, 2-methoxypropylamine was substituted with N-Boc-N-methylethyldiamine.

The Boc deprotection was performed as following: tert-butyl (2-(6-chloro-2-((1S,2R)-2-(6-fluoro-2,3-dimethylphenyl)-1-(5-oxo-4,5-dihydro-1,3,4-oxadiazol-2-yl)propyl)-1,1-dioxido-2,3-dihydro-4H-benzo[e][1,2,4]thiadiazin-4-yl)ethyl)(methyl)carbamate (0.073 mg, 0.117 mmol, 1 eq) and pentamethylbenzene (52 mg, 0.352 mmol, 3 eq) were dissolved in anhydrous DCM (1.2 mL) and the mixture was cooled in an ice-cold water bath. Boron trifluoride diethyl etherate (BF3·Et2O, 0.029 mL, 0.235 mmol, 2 eq) was diluted with DCM and the resulting solution was added dropwise. The reaction was continued for 30 min. The reaction was quenched with sat. NaHCO₃ solution and extracted with DCM. Combined organic fractions were dried, filtered and concentrated. Crude was purified by pHPLC to afford 5-((1S,2R)-1-(6-chloro-4-(2-(methylamino)ethyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (28 mg, yield: 44%). ¹H NMR (400 MHz, Methanol-d₄) δ 7.62 (d, J=8.4 Hz, 1H), 7.00 (dd, J=8.5, 5.7 Hz, 1H), 6.90-6.82 (m, 2H), 6.73 (dd, J=12.1, 8.4 Hz, 1H), 5.47 (d, J=14.5 Hz, 1H), 5.47-5.39 (m, 1H), 5.12 (d, J=14.4 Hz, 1H), 3.90 (dq, J=14.3, 7.3 Hz, 1H), 3.66 (dt, J=14.6, 7.0 Hz, 1H), 3.49-3.39 (m, 1H), 3.06 (t, J=7.2 Hz, 2H), 2.67 (s, 3H), 2.38 (s, 3H), 2.22 (s, 3H), 1.47 (d, J=6.6 Hz, 3H). LC-MS: m/z=523.92 [M+H]⁺; 522.01 [M−H]−.

### Example 116: 5-((1S,2R)-1-(6-chloro-4-methoxy-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

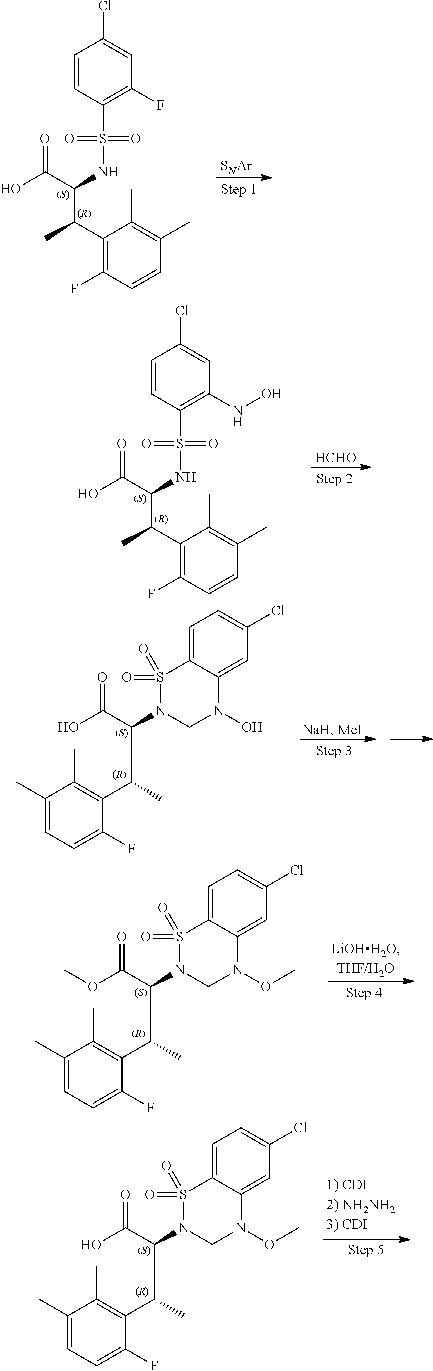

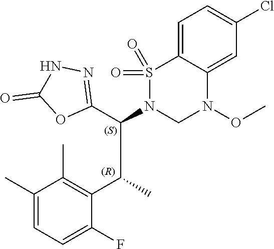

### Step 1: (2S,3R)-2-[4-chloro-2-(hydroxyamino)benzenesulfonamido]-3-(6-fluoro-2,3-dimethylphenyl)butanoic acid

The title compound was synthesized following the procedure described in Ex 30 (step 1 and 2) substituting 2-(methoxypropyl)amine with hydroxylamine solution (50% wt in water). The reaction was carried out at 95° C. for 2 days.

### Step 2: (2S,3R)-2-(6-chloro-4-hydroxy-1,1-dioxido-3,4-dihydro-2H-benzo[e][1,2,4]thiadiazin-2-yl)-3-(6-fluoro-2,3-dimethylphenyl)butanoic acid

The title compound was synthesized following the procedure described in Ex 30 (step 3) using (2S,3R)-2-[4-chloro-2-(hydroxyamino)benzenesulfonamido]-3-(6-fluoro-2,3-dimethylphenyl)butanoic acid.

The reaction was carried out at 95° C. for 1 day.

### Step 3: methyl (2S,3R)-2-(6-chloro-4-methoxy-1,1-dioxido-3,4-dihydro-2H-benzo[e][1,2,4]thiadiazin-2-yl)-3-(6-fluoro-2,3-dimethylphenyl)butanoate

To a cooled solution of (2S,3R)-2-(6-chloro-4-hydroxy-1,1-dioxido-3,4-dihydro-2H-benzo[e][1,2,4]thiadiazin-2-yl)-3-(6-fluoro-2,3-dimethylphenyl)butanoic acid in anhydrous DMF (0.6 mL) at 0° C., NaH 60% in mineral oil (5 mg, 0.23 mmol, 2.1 eq) was added. The mixture was stirred for 5 min. Then, Mel (0.028 mL, 0.45 mmol, 4.5 eq) was added dropwise. The mixture was allowed to reach rt and was continued for 2 h. Upon completion, the reaction was cooled down and quenched with 10% NaH₂PO₄ aqueous solution and extracted with AcOEt. Organic fractions were combined, dried, filtered and concentrated to afford methyl (2S,3R)-2-(6-chloro-4-methoxy-1,1-dioxido-3,4-dihydro-2H-benzo[e][1,2,4]thiadiazin-2-yl)-3-(6-fluoro-2,3-dimethylphenyl)butanoate without further purification.

### Step 4: (2S,3R)-2-(6-chloro-4-methoxy-1,1-dioxido-3,4-dihydro-2H-benzo[e][1,2,4]thiadiazin-2-yl)-3-(6-fluoro-2,3-dimethylphenyl)butanoic acid

To a solution of methyl (2S,3R)-2-(6-chloro-4-methoxy-1,1-dioxido-3,4-dihydro-2H-benzo[e][1,2,4]thiadiazin-2-yl)-3-(6-fluoro-2,3-dimethylphenyl)butanoate (0.055 g, 0.026 mmol, 1.0 eq) in THF (0.5 mL) and water (0.5 mL), LiOH monohydrate (69 mg, 1.60 mmol, 10 eq) was added. The reaction was continued at rt for 4 days. Upon complete consumption of starting material, the mixture was acidified with 0.5M HCl to pH 1-2 and extracted with AcOEt. Organic fractions were combined, dried, filtered and concentrated. The residue was purified by FCC (10% MeOH in DCM) to afford (2S,3R)-2-(6-chloro-4-methoxy-1,1-dioxido-3,4-dihydro-2H-benzo[e][1,2,4]thiadiazin-2-yl)-3-(6-fluoro-2,3-dimethylphenyl)butanoic acid as a white solid.

### Step 5: 5-((1S,2R)-1-(6-chloro-4-methoxy-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized according to procedure described for Ex 30 (step 4). ¹H NMR (400 MHz, Methanol-d₄) δ 7.63 (d, J=8.4 Hz, 1H), 7.13 (d, J=2.0 Hz, 1H), 7.10 (dd, J=8.4, 2.0 Hz, 1H), 7.01 (dd, J=8.4, 5.7 Hz, 1H), 6.74 (dd, J=12.1, 8.4 Hz, 1H), 5.63 (dd, J=11.8, 1.9 Hz, 1H), 5.26 (d, J=13.9 Hz, 1H), 5.21 (d, J=13.8 Hz, 1H), 3.95 (dq, J=13.3, 6.8 Hz, 1H), 3.78 (s, 3H), 2.41 (s, 3H), 2.24 (s, 3H), 1.43 (dd, J=6.9, 1.1 Hz, 3H). LC-MS: m/z=495.07 [M−H]−.

### Example 117: 5-((1S,2R)-1-(6-chloro-4-((R)-1-hydroxypropan-2-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

5-((1S,2R)-1-(4-((R)-1-(benzyloxy)propan-2-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one was synthesized following the procedure used for example 30. For Step 1, 2-methoxypropylamine was substituted with (2R)-1-(benzyloxy)propan-2-amine which was synthesized using the following procedure:

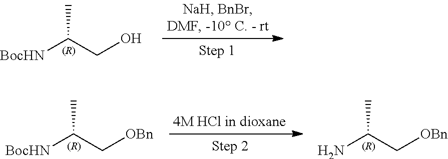

### Step 1. tert-butyl N-[(2R)-1-(benzyloxy)propan-2-yl]carbamate

To a cooled solution of Boc-D-Alaninol (3.0 g, 17.1 mmol, 1.0 eq) in anhydrous DMF (86 mL), NaH 60% in mineral oil (1.50 g, 37.7 mmol, 2.2 eq) was added portionwise at −10° C. After 10 min, benzyl bromide (3.1 g, 18 mmol, 1.05 eq) was added dropwise. Cooling bath was removed, and the reaction was continued at rt for 30 min. The reaction was monitored by LC-MS and TLC. Upon completion, the reaction was cooled down and water was slowly added. Then, the mixture was diluted in MTBE and transferred into a separatory funnel. The layers were separated. Organic layer was washed with 1M LiCl solution. Organic layer was dried, filtered and concentrated in vacuo. The residue was purified by FCC (20% AcOEt in Cyclohexane) to afford tert-butyl N-[(2R)-1-(benzyloxy)propan-2-yl]carbamate (3.6875 g, yield 77%) as a colorless oil.

### Step 2. (2R)-1-(benzyloxy)propan-2-amine

To a solution of tert-butyl N-[(2R)-1-(benzyloxy)propan-2-yl]carbamate (3.687 g, 13.2 mmol, 1.0 eq) in anhydrous 1,4-dioxane (16.5 mL), 4.0 M HCl in dioxane (33.0 mL, 132.0 mmol, 10.0 eq) was added dropwise. The reaction was continued at rt for 2 h. Over the course of reaction, white solid precipitated forming a thick suspension. Reaction progress was monitored by TLC. Volatiles were removed in vacuo.

White residue was taken up in a mixture of MeOH and water, cooled down and solid K₂CO₃ and KOH were added portionwise until pH 12-14 was reached. Solvents were removed in vacuo. The residue was suspended in DCM, drying agent (Na₂SO₄) was added. The solids were filtered off and washed with DCM. DCM was removed in vacuo to give (2R)-1-(benzyloxy)propan-2-amine as a yellow oil (1.9 g, yield 78%).

Deprotection of 5-((1S,2R)-1-(4-((R)-1-(benzyloxy)propan-2-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one was performed as following:

To a cooled solution of 5-((1S,2R)-1-(4-((R)-1-(benzyloxy)propan-2-yl)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (0.063 g, 0.097 mmol, 1.0 eq) and pentamethylbenzene (0.058 g, 0.389 mmol, 4.0 eq) in anhydrous DCM (2 mL), 1M BCl₃ in DCM (0.146 mL, 0.146 mmol, 1.5 eq) was added dropwise. The reaction was continued at −10° C. for 45 min. Upon completion, the mixture was poured into sat. NaHCO₃ solution and extracted with DCM. Combined organic fractions were dried, filtered, and concentrated. Crude was purified by pHPLC to afford 5-((1S,2R)-1-(6-chloro-4-((R)-1-hydroxypropan-2-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (22 mg, yield: 42%). ¹H NMR (300 MHz, Methanol-d₄) δ 7.55 (d, J=8.4 Hz, 1H), 6.99 (dd, J=8.5, 5.9 Hz, 1H), 6.96 (d, J=1.9 Hz, 1H), 6.79-6.68 (m, 2H), 5.33 (dd, J=11.8, 1.8 Hz, 1H), 5.31 (d, J=14.4 Hz, 1H), 5.24 (d, J=14.4 Hz, 1H), 4.08-3.93 (m, 2H), 3.85-3.71 (m, 2H), 2.35 (s, 3H), 2.21 (s, 3H), 1.45 (dd, J=6.9, 1.1 Hz, 3H), 1.38 (d, J=6.8 Hz, 3H). LC-MS: m/z=525.07 [M+H]⁺; 523.05 [M−H]−.

### Example 118: 5-((1S,2R)-1-(6-chloro-1,1-dioxido-4-(3-oxocyclobutyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized by oxidation of 5-((1S,2R)-1-(6-chloro-4-(-3-hydroxycyclobutyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one (see ex 112). Dess-Martin periodinane 35 mg (0.084 mmol, 1.5 eq) was added to a solution of 5-((1S,2R)-1-(6-chloro-4-(3-hydroxycyclobutyl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one in 1 ml DCM at 0° C. The reaction is stirred 1 hr at RT and the crude directly purified by HPLC yielding 5 mg of 5-((1S,2R)-1-(6-chloro-1,1-dioxido-4-(3-oxocyclobutyl)-3,4-dihydro-2H-benzo[e][1,2,4]thiadiazin-2-yl)-2-(6-fluoro-2,3-dimethylphenyl)propyl)-1,3,4-oxadiazol-2 (3H)-one (yield=10%). ¹H NMR (400 MHz, DMSO-d₆) δ 12.08 (s, 1H), 7.64 (d, J=8.4 Hz, 1H), 7.01 (dd, J=8.4, 5.8 Hz, 1H), 6.93 (d, J=1.9 Hz, 1H), 6.90 (dd, J=8.4, 1.8 Hz, 1H), 6.81 (dd, J=12.2, 8.4 Hz, 1H), 5.52 (d, J=14.9 Hz, 1H), 5.35-5.26 (m, 2H), 4.40 (p, J=7.7 Hz, 1H), 4.01 (dq, J=13.6, 6.9 Hz, 1H), 3.81 (dd, J=17.5, 7.5 Hz, 1H), 3.44-3.34 (m, 2H), 3.28-3.20 (m, 1H), 2.31 (s, 3H), 2.17 (s, 3H), 1.36 (d, J=6.5 Hz, 3H). LC-MS: m/z=533.13 [M−H]−.

### Example 119: 5-((1S,2R)-1-(9-acetyl-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

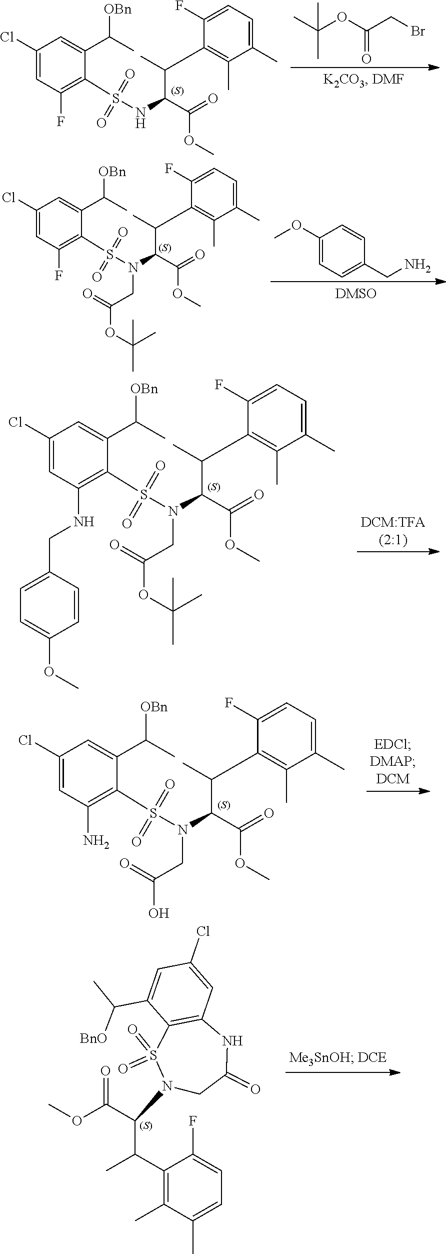

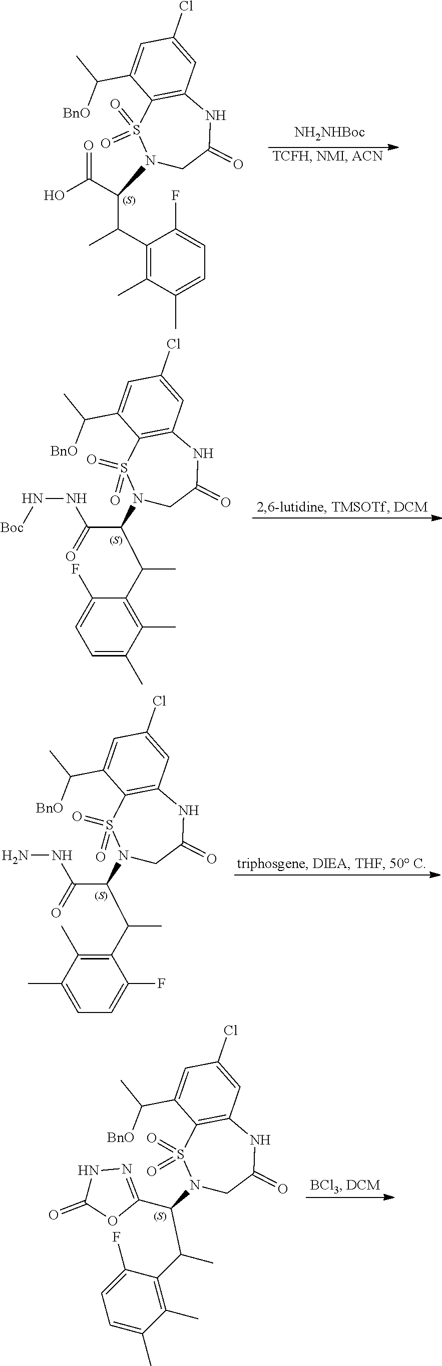

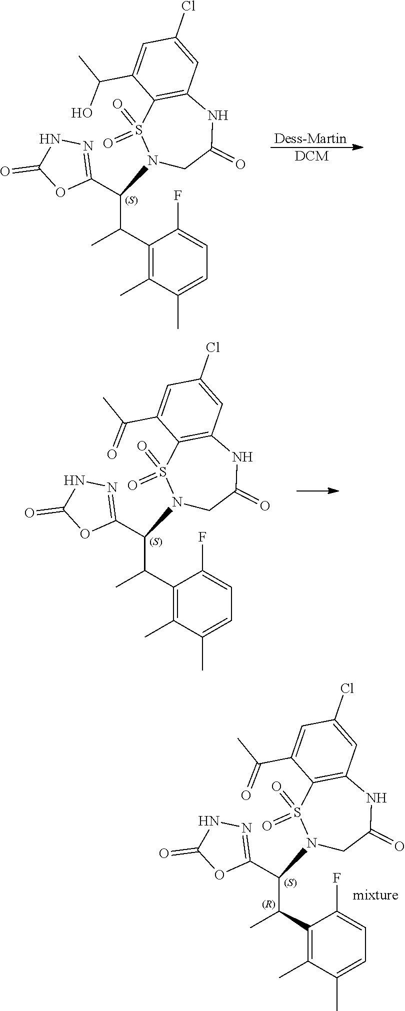

### Step 1: Synthesis of methyl (2S)-2-((2-(1-(benzyloxy)ethyl)-N-(2-(tert-butoxy)-2-oxoethyl)-4-chloro-6-fluorophenyl)sulfonamido)-3-(6-fluoro-2,3-dimethylphenyl)butanoate

To a stirred solution of methyl (2S)-2-(2-[1-(benzyloxy)ethyl]-4-chloro-6-fluorobenzenesulfonamido-3-(6-fluoro-2,3-dimethylphenyl)butanoate (see ex 68) (2.57 g, 4.54 mmol, 1 equiv) and K2CO3 (1.88 g, 13.6 mmol, 3 equiv) in DMF (20 mL) was added tert-butyl 2-bromoacetate (794 μL, 5.5 mmol, 1.2 equiv) dropwise at room temperature. The resulting mixture was stirred for 2 h at 60° C. The resulting mixture was quenched with water (150 mL). The mixture was extracted with EtOAc (3×200 mL). The combined organic layers were washed with brine (2×300 mL), dried over anhydrous Na₂SO₄. After filtration, the filtrate was concentrated under reduced pressure. The crude product was used in the next step directly without further purification.

### Step 2: Synthesis of methyl (2S)-2-((2-(1-(benzyloxy)ethyl)-N-(2-(tert-butoxy)-2-oxoethyl)-4-chloro-6-((4-methoxybenzyl)amino)phenyl)sulfonamido)-3-(6-fluoro-2,3-dimethylphenyl)butanoate

To a stirred solution of methyl (2S)-2-(N-[2-(tert-butoxy)-2-oxoethyl]2-[1-(benzyloxy)ethyl]-4-chloro-6-fluorobenzenesulfonamido-3-(6-fluoro-2,3-dimethylphenyl)butanoate (3 g, 4.41 mmol, 1 equiv) in DMSO (100 mL) was added benzenemethanamine, 4-methoxy-(6.05 g, 44.1 mmol, 10 equiv) dropwise at room temperature. The resulting mixture was stirred overnight at 60° C. The reaction was quenched with water (50 ml) at room temperature. The resulting mixture was extracted with EtOAc (1×100 mL). The combined organic layers were washed with brine (3×200 mL), dried over anhydrous Na₂SO₄. After filtration, the filtrate was concentrated under reduced pressure. The residue was purified by silica gel column chromatography, eluted with PEI/EA (80:20) to afford methyl (2S)-2-(N-[2-(tert-butoxy)-2-oxoethyl]2-[1-(benzyloxy)ethyl]-4-chloro-6-([(4-methoxyphenyl)methyl]aminobenzenesulfonamido-3-(6-fluoro-2,3-dimethylphenyl)butanoate (1.33 g, 37.8%) as a colorless oil.

### Step 3: Synthesis of N-((2-amino-6-(1-(benzyloxy)ethyl)-4-chlorophenyl)sulfonyl)-N-((2S)-3-(6-fluoro-2,3-dimethylphenyl)-1-methoxy-1-oxobutan-2-yl)glycine

To a stirred solution of methyl (2S)-2-(N-[2-(tert-butoxy)-2-oxoethyl]2-[1-(benzyloxy)ethyl]-4-chloro-6-([(4-methoxyphenyl)methyl]aminobenzenesulfonamido-3-(6-fluoro-2,3-dimethylphenyl)butanoate (1.33 g, 1.7 mmol, 1 equiv) in DCM (15 mL) was added TFA (7 mL) dropwise at room temperature. The resulting mixture was stirred for 5 h at room temperature. The resulting mixture was concentrated under vacuum. The residue was purified by silica gel column chromatography, eluted with PE/EA (55:45) to afford (N-[(2S)-3-(6-fluoro-2,3-dimethylphenyl)-1-methoxy-1-oxobutan-2-yl]2-amino-6-[1-(benzyloxy)ethyl]-4-chlorobenzenesulfonamidoacetic acid (760 mg, 73.4%) as a white solid.

### Step 4: Synthesis of methyl (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoate

To a stirred solution of (N-[(2S)-3-(6-fluoro-2,3-dimethylphenyl)-1-methoxy-1-oxobutan-2-yl]2-amino-6-[1-(benzyloxy)ethyl]-4-chlorobenzenesulfonamidoacetic acid (760 mg, 1.22 mmol, 1 equiv) in DCM (10 mL) were added EDCI (258 mg, 1.35 mmol, 1.1 equiv) and DMAP (15 mg, 0.122 mmol, 0.1 equiv) in small portions at room temperature. The resulting mixture was stirred for 1 h at room temperature. The resulting mixture was concentrated under vacuum. The residue was purified by silica gel column chromatography, eluted with PE/EA (70:30) to afford methyl (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoate (611 mg, 82.8%) as a white solid.

### Step 5: Synthesis of (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoic acid

To a stirred solution of methyl (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoate (595 mg, 0.987 mmol, 1 equiv) in DCE (6 mL) were added trimethyltin hydroxide (3.57 g, 19.7 mmol, 20 equiv) in portions at room temperature. The resulting mixture was stirred overnight at 80° C. The resulting mixture was filtered, the filter cake was washed with EtOAc (3×20 mL). The filtrate was concentrated under reduced pressure.

The residue was purified by reversed-phase flash chromatography with the following conditions: column, C18 silica gel; mobile phase, MeCN in Water (0.1% FA), 0% to 100% gradient in 20 min; detector, UV 220 nm. This resulted in (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoic acid (260 mg, 44.7%6) as an off-white solid.

### Step 6: Synthesis of tert-butyl 2-((2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate

Into a 50 mL round-bottom flask were added (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoic acid (250 mg, 0.424 mmol, 1 equiv), ACN (6 mL), tert-butoxycarbohydrazide (84.13 mg, 0.636 mmol, 1.5 equiv), N,N,N′,N′-tetramethylchloroformamidinium hexafluorophosphate (595.4 mg, 2.12 mmol, 5 equiv) and N-methylimidazole (348.5 mg, 4.24 mmol, 10 equiv) at room temperature. The resulting mixture was stirred for 1 h at room temperature under air atmosphere. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in of tert-butyl 2-((2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate (160 mg, 53.6%) as a white solid.

### Step 7: (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanehydrazide

Into a 50 mL round-bottom flask were added tert-butyl 2-((2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanoyl)hydrazine-1-carboxylate (160 mg, 0.228 mmol, 1 equiv) and DCM (4.8 mL) at room temperature. To the above mixture was added 2,6-Lutidine (530.01 μL, 4.56 mmol, 20 equiv) and TMSOTF (658.9 μL, 3.65 mmol, 16 equiv) dropwise at 0° C. The resulting mixture was stirred for additional 1 h at room temperature. The resulting mixture was concentrated under reduced pressure. The residue was purified by reversed-phase flash chromatography with the following conditions: column, C18 silica gel; mobile phase, MeCN in Water (0.1% FA), 50% to 60/gradient in 10 min; detector, UV 254 nm. This resulted in (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanehydrazide (110 mg, 80.2%) as a white solid.

### Step 8: 5-((1S)-1-(9-(1-(benzyloxy)ethyl)-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

Into a 20 mL sealed tube were added (2S)-2-(9-(1-(benzyloxy)ethyl)-7-chloro-1,1-dioxido-4-oxo-4,5-dihydrobenzo[f][1,2,5]thiadiazepin-2 (3H)-yl)-3-(6-fluoro-2,3-dimethylphenyl)butanehydrazide (110 mg, 0.18 mmol, 1 equiv), DCM (3 mL) and triphosgene (27.1 mg, 0.091 mmol, 0.5 equiv) at room temperature.

The resulting mixture was stirred for 1 h at room temperature under air atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-((1S)-1-(9-(1-(benzyloxy)ethyl)-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (80 mg, 69.7%) as a white solid. Step 9: 5-((1S)-1-(7-chloro-9-(1-hydroxyethyl)-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

Into a 50 mL round-bottom flask were added 5-((1S)-1-(9-(1-(benzyloxy)ethyl)-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (46 mg, 0.073 mmol, 1 equiv) and DCM (1.38 mL) at room temperature. To the above mixture was added BCl3 (292 μL, 0.29 mmol, 4 equiv) dropwise at 0° C. The resulting mixture was stirred for additional 1 h at 0° C. The reaction was quenched with water (10 ml) at room temperature. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-((1S)-1-(7-chloro-9-(1-hydroxyethyl)-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (30 mg, 76.12%) as a white solid.

### Step 10: 5-((1S,2R)-1-(9-acetyl-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one

Into a 50 mL round-bottom flask were added 5-((1S)-1-(7-chloro-9-(1-hydroxyethyl)-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (30 mg, 0.056 mmol, 1 equiv) and DCM (0.67 mL) at room temperature. To the above mixture was added Dess-Martin reagent (47.2 mg, 0.11 mmol, 2 equiv) in portions at room temperature. The resulting mixture was stirred for additional 0.5 h at room temperature. The reaction was quenched with water (10 ml) at room temperature. The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in 5-((1S)-1-(9-acetyl-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (20 mg, 66.9%).

The mixture of diastereoisomers was fractionated by reversed-phase flash chromatography with the following conditions: Column: XselectCSH Prep OBD CIS Column, 30*150 mm, 5 μm; Mobile Phase A: Water (0.1% FA), Mobile Phase B: ACN; Flow rate: 60 mL/min mL/min; Gradient: 46% B to 61% B in 8 min; Wave Length: 254 nm/220 nm nm; RT1 (min): 6.106.77. This resulted in: 5-((1S,2R)-1-(9-acetyl-7-chloro-1,1-dioxido-4-oxo-4,5-dihydrobenzo[f][1,2,5]thiadiazepin-2 (3H)-yl)-2-(6-fluoro-2,3-dimethylphenyl)propyl)-1,3,4-oxadiazol-2 (3H)-one (6.1 mg, 33.58%). LCMS:(ES,m/z): [M+H]+=537.05. ¹H NMR (400 MHz, Methanol-d₄) δ 7.16 (dd, J=18.5, 1.9 Hz, 2H), 7.03 (dd, J=8.4, 5.8 Hz, 1H), 6.77 (dd, J=12.2, 8.4 Hz, 1H), 5.24 (dd, J=11.7, 2.3 Hz, 1H), 4.75-4.53 (m, 2H), 3.88 (ddd, J=13.2, 7.4, 3.8 Hz, 1H), 2.63 (s, 3H), 2.29 (s, 3H), 2.21 (s, 3H), 1.42 (dd, J=6.9, 1.2 Hz, 3H).

### Example 120: 5-((1S,2R)-1-(6-chloro-4-hydroxy-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title 5-((1S,2R)-1-(6-chloro-4-hydroxy-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one was synthesized from 5-((1S,2R)-1-(4-(benzyloxy)-6-chloro-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one. Benzyl deprotection was carried out according to procedure described for Ex 50. ¹H NMR (400 MHz, Methanol-d₄) 57.57 (d, J=8.4 Hz, 1H), 7.12 (d, J=2.0 Hz, 1H), 7.03-6.99 (m, 1H), 6.97 (dd, J=8.5, 2.0 Hz, 1H), 6.73 (dd, J=12.1, 8.4 Hz, 1H), 5.54 (dd, J=11.6, 1.6 Hz, 1H), 5.31 (d, J=13.8 Hz, 1H), 5.10 (d, J=13.8 Hz, 1H), 3.88 (dq, J=13.3, 6.8 Hz, 1H), 2.38 (s, 3H), 2.23 (s, 3H), 1.43 (dd, J=7.0, 1.1 Hz, 3H). LC-MS: m/z=481.07 [M−H]−.

### Example 121: 5-((1S,2R)-1-(6-chloro-4-((S)-1-hydroxypropan-2-yl)-1,1-dioxido-3,4-dihydro-2H-benzo[e][1,2,4]thiadiazin-2-yl)-2-(6-fluoro-2,3-dimethylphenyl)propyl)-1,3,4-oxadiazol-2 (3H)-one

The title compound was synthesized following procedure described for example 117. For Step 1, 2-methoxypropylamine was substituted with (2S)-1-(benzyloxy)propan-2-amine.

¹H NMR (300 MHz, Methanol-d₄) δ 7.55 (d, J=8.4 Hz, 1H), 6.99 (dd, J=8.4, 5.8 Hz, 1H), 6.92 (d, J=1.8 Hz, 1H), 6.79-6.67 (m, 2H), 5.45 (dd, J=11.8, 1.8 Hz, 1H), 5.34 (d, J=14.4 Hz, 1H), 5.20 (d, J=14.4 Hz, 1H), 4.12-4.01 (m, 1H), 4.01-3.87 (m, 1H), 3.74-3.65 (m, 2H), 2.37 (s, 3H), 2.22 (s, 3H), 1.49-1.42 (m, 3H), 1.38 (d, J=6.9 Hz, 3H). LC-MS: m/z=523.13 [M−H]−.

### Example A: RNR Enzyme Activity

A rapid-fire mass spectrometry (RF/MS) assay was used to assess RNR enzyme activity using a 384 well plate and a robotic platform.

The plate layout included two validated reference compounds (Triapine (3-AP) and Hydroxyurea (HU)):
A dose response in duplicate; top concentration: 5 μM (3-AP) and 250 μM (HU), semi-log dilutions.
Spike wells in triplicate randomly spotted at four concentrations:
   250 μM, 100 μM, 30 μM and 2 μM for HU
   5 μM, 2 μM, 0.6 μM and 0.04 μM for 3-AP

First, the multidrop pipes were saturated for 30 minutes with enzymatic solution. Then 30 μL of Stop solution was distributed in column 24. Next, 15 μL of enzyme was distributed in column 1 to 24. Next, a pre-incubation step of 15 minutes at room temperature occurred, followed by distribution of 15 μL of substrate solution (column 1 to 24). Next, the plate was incubated for 45 minutes at 37° C. 30 μL of Stop solution was then distributed to columns 1 to 23.

The final parameters for the enzyme reactions were:
Incubation: 37° C., 45 min
[CDP]: 5 μM; [ATP]: 1 mM; [NADPH]: No
[RNR]final: 50 nM with 1:1 (RNR1:RNR2) ratio
Final volume: 30 μL
Stop solution: 6% HCOOH in water

The compounds were screened at concentrations up to 50 μM concentrations and the results are shown in table 3.

TABLE 3

| Example | RF/MS IC₅₀ |
|---|---|
| 1 | A |
| 2 | A |
| 3 | A |
| 4 | C |
| 5 | B |
| 6 | B |
| 7 | B |
| 8 | B |
| 9 | B |
| 10 | A |
| 11 | NT |
| 12 | B |
| 13 | B |
| 14 | B |
| 15 | C |
| 16 | B |
| 17 | D |
| 18 | C |
| 19 | B |
| 20 | A |
| 21 | B |
| 22 | A |
| 23 | A |
| 24 | C |
| 25 | A |
| 26 | B |
| 27 | D |
| 28 | D |
| 29 | E |
| 30 | A |
| 31 | NT |
| 32 | B |
| 33 | A |
| 34 | A |
| 35 | B |
| 36 | A |
| 37 | D |
| 38 | A |
| 39 | E |
| 40 | A |
| 41 | C |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | B |
| 58 | B |
| 59 | A |
| 60 | A |
| 61 | C |
| 62 | A |
| 63 | A |
| 64 | B |
| 65 | A |
| 66 | B |
| 67 | C |
| 68 | B |
| 69 | D |
| 70 | B |
| 71 | C |
| 72 | A |
| 73 | A |
| 74 | A |
| 75 | A |
| 76 | A |
| 77 | C |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | NT |
| 90 | NT |
| 91 | NT |
| 92 | NT |
| 93 | NT |
| 94 | NT |
| 95 | A |
| 96 | A |
| 97 | A |
| 98 | A |
| 99 | B |
| 100 | B |
| 101 | B |
| 102 | A |
| 103 | D |
| 104 | A |
| 105 | A |
| 106 | A |
| 107 | NT |
| 108 | NT |
| 109 | NT |
| 110 | NT |
| 111 | NT |
| 112 | NT |
| 113 | NT |
| 114 | NT |
| 115 | NT |
| 116 | NT |
| 117 | NT |
| 118 | NT |
| 119 | NT |
| 120 | NT |
| 121 | NT |

A: 0 μM < IC₅₀ ≤ 20 nM;
B: 20 nM < IC₅₀ ≤ 100 nM
C: 100 nM < IC₅₀ ≤ 1 μM
D: 1 μM < IC₅₀ ≤ 10 μM
E: 10 μM < IC₅₀ ≤ 100 μM
NT: not tested

### Example B: Alphalisa Assay

Colo320 DM cells (ATCC #CCL-220, derived from human colorectal adenocarcinoma, Dukes' type C) were seeded on a %-well, cell culture treated assay plate at a density of 50,000 cells/well in 200 μL of RPMI-1640 media supplemented with 10% Fetal Bovine Serum and incubated at 37° C. overnight. The following day, test compound dilutions were added directly to the plated cells by a Tecan digital dispenser to a final DMSO concentration of <0.5%. and incubated at 37° C. overnight (approximately 16 hours). The following day all cell culture media was removed from the cells. 75 μL of 1×AlphaLisa lysis buffer was added to each well and plates were agitated on a shaker for 30 minutes at room temperature. The lysis of cells and detection of pCHK1 (S345) were performed with reagents contained within the AlphaLisa Sure Fire assay kit (Perkin Elmer #ALSU-PCHK1-A) according to the manufacturer's instructions. 10 μL of each lysate was then transferred to a white, 384-well assay plate (Perkin Elmer #6008280). 5 μL of Acceptor mix was then added to each well of lysate in the white, 384-well assay plate and incubated in the dark at room temperature for 60 minutes. 5 μL of Donor mix was then added to each well of the white, 384-well assay plate in subdued light and incubated at room temperature for 60 minutes. Plates were read on an Alpha Technology-compatible plate reader using standard AlphaLisa settings.

The results are shown in table 4.

TABLE 4

| Example | Alphalisa IC₅₀ |
|---|---|
| 1 | A |
| 2 | B |
| 3 | A |
| 4 | B |
| 5 | B |
| 6 | A |
| 7 | A |
| 8 | A |
| 9 | A |
| 10 | A |
| 11 | D |
| 12 | C |
| 13 | B |
| 14 | B |
| 15 | E |
| 16 | B |
| 17 | E |
| 18 | D |
| 19 | B |
| 20 | B |
| 21 | B |
| 22 | A |
| 23 | B |
| 24 | E |
| 25 | B |
| 26 | D |
| 27 | D |
| 28 | E |
| 29 | E |
| 30 | A |
| 31 | B |
| 32 | D |
| 33 | A |
| 34 | B |
| 35 | D |
| 36 | B |
| 37 | E |
| 38 | A |
| 39 | E |
| 40 | C |
| 41 | E |
| 42 | A |
| 43 | A |
| 44 | A |
| 45 | A |
| 46 | A |
| 47 | B |
| 48 | A |
| 49 | A |
| 50 | A |
| 51 | A |
| 52 | A |
| 53 | B |
| 54 | B |
| 55 | B |
| 56 | B |
| 57 | D |
| 58 | D |
| 59 | C |
| 60 | A |
| 61 | D |
| 62 | A |
| 63 | A |
| 64 | A |
| 65 | A |
| 66 | D |
| 67 | C |
| 68 | A |
| 69 | E |
| 70 | B |
| 71 | A |
| 72 | A |
| 73 | A |
| 74 | A |
| 75 | A |
| 76 | B |
| 77 | E |
| 78 | A |
| 79 | A |
| 80 | A |
| 81 | A |
| 82 | A |
| 83 | A |
| 84 | A |
| 85 | B |
| 86 | B |
| 87 | A |
| 88 | A |
| 89 | A |
| 90 | A |
| 91 | A |
| 92 | B |
| 93 | A |
| 94 | B |
| 95 | A |
| 96 | A |
| 97 | A |
| 98 | A |
| 99 | A |
| 100 | B |
| 101 | B |
| 102 | A |
| 103 | C |
| 104 | A |
| 105 | B |
| 106 | A |
| 107 | A |
| 108 | A |
| 109 | A |
| 110 | A |
| 111 | A |
| 112 | A |
| 113 | C |
| 114 | C |
| 115 | C |
| 116 | A |
| 117 | B |
| 118 | A |
| 119 | C |
| 120 | B |
| 121 | A |

A: 0 μM ≤ IC₅₀ ≤ 1 μM;
B: 1 μM < IC₅₀ ≤ 5 μM
C: 5 μM < IC₅₀ ≤ 10 μM
D: 10 μM < IC₅₀ ≤ 50 μM
E: 50 μM < IC₅₀
NT: not tested

### Example C: Human Hepatocyte Stability Assay

1. 10 mM stock solutions of test compounds and positive control were prepared in DMSO. Thawing medium and supplement incubation medium (serum-free) were placed in a 37° C. water bath for at least 15 minutes prior to use.

2. Stock solutions were diluted to 100 μM by combining 198 μL acetonitrile and 2 μL of 10 mM stock solution. Verapamil was use as positive control in the assay.

3. Vials of cryopreserved human hepatocytes were removed from storage, while ensuring that vials remain at cryogenic temperatures. The pressure was removed by loosening and re-tightening the cap. The vials were thawed in a 37° C. water bath with gentle shaking. Vials remained in the water bath until all ice crystals had dissolved and were no longer visible. Vials were sprayed with 70% ethanol before being transferred to a biosafety cabinet. Then the contents were poured into a 50 mL thawing medium conical tube. Vials were centrifuged at 100 g for 10 minutes at room temperature. Thawing medium was aspirated and hepatocytes were re-suspended with serum-free incubation medium to yield ˜1.5×10⁶ cells/mL.

4. Cell viability and density were counted using Cellometer® Vision, and then cells were diluted with serum-free incubation medium to a working cell density of 0.5×10⁶ viable cells/ml.

5. Aliquots of 198 μL hepatocytes were dispensed into each well of a 96-well non-coated plate. The plate was placed in the incubator on an orbital shaker at 500 rpm for approximately 10 minutes.

6. Aliquots of 2 μL of the 100 μM test compounds or verapamil were added into respective wells of the non-coated 96-well plate to start the reaction. This assay was performed in duplicate. The plate was incubated in the incubator on an orbital shaker at 500 rpm for the designed time points.

7.25 μL of contents were transferred and mixed with 5 volumes (125 μL) of cold acetonitrile with IS (100 nM alprazolam, 200 nM caffeine, 100 nM labetalol and 100 nM tolbutamide) to terminate the reaction at time points of 0.5 and 60 minutes. Samples were centrifuged for 30 minutes at 3,220 g. Then transfer 100 μL of the supernatant to new 96-well plates for analysis. Add 100 μL of distilled water to each sample and mix for analysis by LC-MS/MS.

### Data Analysis

All calculations were carried out using Microsoft Excel. Peak areas were determined from extracted ion chromatograms. The in vitro half-life (tin) of the parent compound was determined by regression analysis of the percent parent disappearance vs. time curve.
a. The in vitro half-life (in vitro tin) was determined from the slope value: in vitro t_{1/2}=0.693/k
b. Conversion of the in vitro tin (in min) into the in vitro intrinsic clearance (in vitro CLᵢₙₜ, in μL/min/10⁶ cells) was done using the following equation:

$$in vitro {CL}_{int} = kV / N$$

$$V = incubation volume ( 0.2 mL ) ;$$

$$N = number of hepatocytes per well ( 0.1 \times {10}^{6} cells ) .$$

$$c . {Cl}_{int} ( mL / min / kg ) = ( 0.693 / T 1 / 2 ) \times ( 1 / hepatocyte density ) \times Scaling Factors$$

$$d . {Cl}_{hep} = ( QH \times {Cl}_{int} \times fub ) / ( QH + {Cl}_{int} \times fub ) .$$

The results are shown in table 5.

TABLE 5

| Ex. | SPECIES | Clhep |
|---|---|---|
| 1 | human | A |
| 2 | human | A |
| 3 | human | B |
| 8 | human | B |
| 48 | human | A |
| 50 | human | A |
| 60 | human | B |
| 75 | human | B |
| 89 | human | B |
| 90 | human | B |
| 92 | human | A |
| 93 | human | A |
| 116 | human | B |
| 117 | human | B |
| 118 | human | B |
| 120 | human | B |
| 121 | human | B |

A: 0 ml/min/kg ≤ Clhep ≤ 10 ml/min/kg
B: 10 ml/min/kg < Clhep ≤ 20 ml/min/kg
C: 20 ml/min/kg < Clhep

### Example D: Pharmaceutical Compositions

### Example D1: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example D2: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example D3: Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

The examples and embodiments described herein are for illustrative purposes only and in some embodiments, various modifications or changes are to be included within the purview of disclosure and scope of the appended claims.

What is claimed is:

1. A compound of Formula (I), or a pharmaceutically acceptable salt, solvate, tautomer, or stereoisomer thereof: 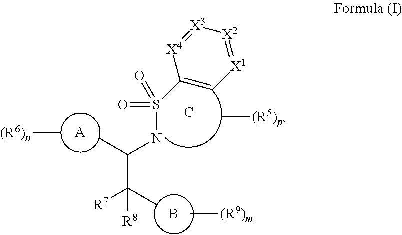
wherein:
X¹ is N or CR¹;
X² is N or CR²;
X³ is N or CR³;
X⁴ is N or CR⁴;
R¹ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R² is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R³ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
R⁴ is hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
Ring C is a 5- to 8-membered heterocycloalkyl comprising one or two additional heteroatoms selected from the group consisting of —O—, —S—, —S(═O)—, —S(═O)₂—, and —NR¹⁰—;
R¹⁰ is hydrogen, —OH, —OR^{A}, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{10a};
each R^{10a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{10a} on the same carbon are taken together to form an oxo;
each R⁵ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R⁵ on the same carbon are taken together to form an oxo;
or two R⁵ on the same carbon are taken together to form a cycloalkyl or heterocycloalkyl; each optionally substituted with one or more R;
or two R⁵ on adjacent atoms are taken together to form a cycloalkyl, heterocycloalkyl, aryl, or heteroaryl;
each optionally substituted with one or more R;
or one R⁵ and R¹⁰ are taken together to form a heterocycloalkyl or heteroaryl; each optionally substituted with one or more R;
p is 0-4;
Ring A is a 5-membered heterocycloalkyl or 5-membered heteroaryl;
each R⁶ is independently deuterium, halogen, —CN, —NO₂, —OH, —OR^{A}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁶ on the same atom are taken together to form an oxo;
n is 0-3;
R⁷ is hydrogen, deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
R⁸ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
Ring B is cycloalkyl, heterocycloalkyl, aryl, or heteroaryl;
each R⁹ is independently deuterium, halogen, —CN, —NO₂, —OH, —OR^{A}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R;
or two R^{9a} on the same atom are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, cycloalkyl, or heterocycloalkyl;
or two R on the same atom are taken together to form an oxo.

2. The compound of claim 1, or a pharmaceutically acceptable salt, solvate, tautomer, or stereoisomer thereof, wherein Ring C is a 6- to 7-membered heterocycloalkyl comprising one additional heteroatom selected from the group consisting of —O—, —S—, and —NR¹⁰—.

3. The compound of claim 1, or a pharmaceutically acceptable salt, solvate, tautomer, or stereoisomer thereof, wherein Ring C is a 6- to 7-membered heterocycloalkyl comprising one additional heteroatom selected from the group consisting of —O— and —NR¹⁰—.

4. The compound of claim 1, or a pharmaceutically acceptable salt, solvate, tautomer, or stereoisomer thereof, wherein Ring C is a 6-membered heterocycloalkyl comprising one additional heteroatom selected from the group consisting of —O—, —S—, and —NR¹⁰—.

5. The compound of claim 1, or a pharmaceutically acceptable salt, solvate, tautomer, or stereoisomer thereof, wherein Ring C is a 6-membered heterocycloalkyl comprising one additional heteroatom selected from the group consisting of —O— and —NR¹⁰—.

6. The compound of claim 1, or a pharmaceutically acceptable salt, solvate, tautomer, or stereoisomer thereof, wherein Ring C is a 6-membered heterocycloalkyl comprising one additional heteroatom that is —NR¹⁰—.

7. The compound of claim 1, or a pharmaceutically acceptable salt, solvate, tautomer, or stereoisomer thereof, wherein Ring C is a 6-membered heterocycloalkyl comprising one additional heteroatom that is —O—.

8. The compound of claim 1, or a pharmaceutically acceptable salt, solvate, tautomer, or stereoisomer thereof, wherein Ring C is a 7-membered heterocycloalkyl comprising one additional heteroatom selected from the group consisting of —O—, —S—, and —NR¹⁰—.

9. The compound of claim 1, or a pharmaceutically acceptable salt, solvate, tautomer, or stereoisomer thereof, wherein Ring C is a 7-membered heterocycloalkyl comprising one additional heteroatom selected from the group consisting of —O— and —NR¹⁰—.

10. The compound of claim 1, or a pharmaceutically acceptable salt, solvate, tautomer, or stereoisomer thereof, wherein Ring C is a 7-membered heterocycloalkyl comprising one additional heteroatom that is —NR¹⁰—.

11. The compound of claim 1, or a pharmaceutically acceptable salt, solvate, tautomer, or stereoisomer thereof, wherein Ring C is a 7-membered heterocycloalkyl comprising one additional heteroatom that is —O—.

12. The compound of any one of claims 1-11, or a pharmaceutically acceptable salt, solvate, tautomer, or stereoisomer thereof, wherein each R⁵ is independently C₁-C₆alkyl or C₁-C₆haloalkyl.

13. The compound of any one of claims 1-12, or a pharmaceutically acceptable salt, solvate, tautomer, or stereoisomer thereof, wherein each R⁵ is independently C₁-C₆alkyl.

14. The compound of any one of claims 1-13, or a pharmaceutically acceptable salt, solvate, tautomer, or stereoisomer thereof, wherein p is 0 or 1.

15. The compound of claim 1, or a pharmaceutically acceptable salt, solvate, tautomer, or stereoisomer thereof, wherein the compound of Formula (I) is of Formula (Ia): 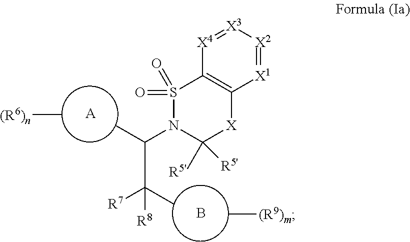
wherein:
X is —O—, —S—, or
each R^{5′} is independently hydrogen or R⁵;
or two R^{5′} on the same carbon are taken together to form an oxo;
or two R^{5′} on the same carbon are taken together to form a cycloalkyl or heterocycloalkyl; each optionally substituted with one or more R;
or one R^{5′} and R¹⁰ are taken together to form a heterocycloalkyl or heteroaryl; each optionally substituted with one or more R.

16. The compound of claim 1, or a pharmaceutically acceptable salt, solvate, tautomer, or stereoisomer thereof, wherein the compound of Formula (I) is of Formula (Ib):
wherein 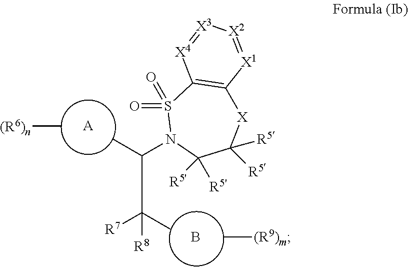
X is —O—, —S—, or —NR¹⁰—;
each R^{5′} is independently hydrogen or R⁵;
or two R^{5′} on the same carbon are taken together to form an oxo;
or two R^{5′} on the same carbon are taken together to form a cycloalkyl or heterocycloalkyl; each optionally substituted with one or more R;
or two R^{5′} on adjacent carbons are taken together to form a cycloalkyl, heterocycloalkyl, aryl, or heteroaryl; each optionally substituted with one or more R;
or one R^{5′} and R¹⁰ are taken together to form a heterocycloalkyl or heteroaryl; each optionally substituted with one or more R.

17. The compound of claim 1, or a pharmaceutically acceptable salt, solvate, tautomer, or stereoisomer thereof, wherein the compound of Formula (I) is of Formula (Ic): 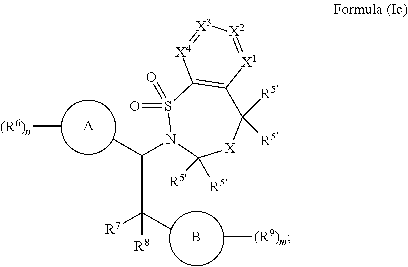
wherein:
X is —O—, —S—, or NR¹⁰—;
each R^{5′} is independently hydrogen or R⁵;
or two R^{5′} on the same carbon are taken together to form an oxo;
or two R^{5′} on the same carbon are taken together to form a cycloalkyl or heterocycloalkyl; each optionally substituted with one or more R;
or one R^{5′} and R¹⁰ are taken together to form a heterocycloalkyl or heteroaryl; each optionally substituted with one or more R.

18. The compound of any one of claims 15-17, or a pharmaceutically acceptable salt, solvate, tautomer, or stereoisomer thereof, wherein X is —O—.

19. The compound of any one of claims 15-17, or a pharmaceutically acceptable salt, solvate, tautomer, or stereoisomer thereof, wherein X is —NR¹⁰—.

20. The compound of any one of claims 1-10, 12-17, or 19, or a pharmaceutically acceptable salt, solvate, tautomer, or stereoisomer thereof, wherein R¹⁰ is hydrogen, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally and independently substituted with one or more R^{10a}.

21. The compound of any one of claims 1-10, 12-17, or 19, or a pharmaceutically acceptable salt, solvate, tautomer, or stereoisomer thereof, wherein R¹⁰ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein the alkyl is optionally and independently substituted with one or more R^{10a}.

22. The compound of any one of claims 1-10, 12-17, or 19, or a pharmaceutically acceptable salt, solvate, tautomer, or stereoisomer thereof, wherein R¹⁰ is C₁-C₆alkyl.

23. The compound of any one of claims 1-22, or a pharmaceutically acceptable salt, solvate, tautomer, or stereoisomer thereof, wherein Ring A is a 5-membered heterocycloalkyl.

24. The compound of any one of claims 1-22, or a pharmaceutically acceptable salt, solvate, tautomer, or stereoisomer thereof, wherein Ring A is a 2,3-dihydro-1,3,4-oxadiazole.

25. The compound of any one of claims 1-22, or a pharmaceutically acceptable salt, solvate, tautomer, or stereoisomer thereof, wherein Ring A is a 5-membered heteroaryl.

26. The compound of any one of claims 1-22, or a pharmaceutically acceptable salt, solvate, tautomer, or stereoisomer thereof, wherein Ring A is a triazole or tetrazole.

27. The compound of any one of claims 1-22, or a pharmaceutically acceptable salt, solvate, tautomer, or stereoisomer thereof, wherein Ring A is a triazole.

28. The compound of any one of claims 1-22, or a pharmaceutically acceptable salt, solvate, tautomer, or stereoisomer thereof, wherein Ring A is a tetrazole.

29. The compound of any one of claims 1-28, or a pharmaceutically acceptable salt, solvate, tautomer, or stereoisomer thereof, wherein each R⁶ is independently deuterium, halogen, —CN, —OH, —OR^{a}, —NR^{c}R^{d}, C₁-C₆alkyl, C₁-C₆haloalkyl, or C₁-C₆deuteroalkyl; or two R⁶ on the same atom are taken together to form an oxo.

30. The compound of any one of claims 1-29, or a pharmaceutically acceptable salt, solvate, tautomer, or stereoisomer thereof, wherein each R⁶ is independently deuterium, halogen, or C₁-C₆alkyl; or two R⁶ on the same atom are taken together to form an oxo.

31. The compound of any one of claims 1-30 or a pharmaceutically acceptable salt, solvate, tautomer, or stereoisomer thereof, wherein two R⁶ on the same atom are taken together to form an oxo.

32. The compound of any one of claims 1-31, or a pharmaceutically acceptable salt, solvate, tautomer, or stereoisomer thereof, wherein n is 0 or 1.

33. The compound of any one of claims 1-31, or a pharmaceutically acceptable salt, solvate, tautomer, or stereoisomer thereof, wherein n is 2 or 3.

34. The compound of any one of claims 1-22, or a pharmaceutically acceptable salt, solvate, tautomer, or stereoisomer thereof, wherein 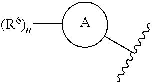
is

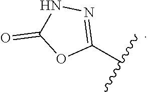 [SMILES: *c1n[nH]c(=O)o1]

35. The compound of any one of claims 1-34, or a pharmaceutically acceptable salt, solvate, tautomer, or stereoisomer thereof, wherein X¹ is CR¹.

36. The compound of any one of claims 1-35, or a pharmaceutically acceptable salt, solvate, tautomer, or stereoisomer thereof, wherein R¹ is hydrogen, deuterium, halogen, —CN, —OH, —OR^{a}, —NR^{c}R^{d}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl.

37. The compound of any one of claims 1-36, or a pharmaceutically acceptable salt, solvate, tautomer, or stereoisomer thereof, wherein R¹ is hydrogen, deuterium, halogen, —C(═O)R^{a}, C₁-C₆alkyl, C₁-C₆haloalkyl, or C₁-C₆hydroxyalkyl.

38. The compound of any one of claims 1-37, or a pharmaceutically acceptable salt, solvate, tautomer, or stereoisomer thereof, wherein R¹ is hydrogen, —C(═O)R^{a}, C₁-C₆alkyl, or C₁-C₆hydroxyalkyl.

39. The compound of any one of claims 1-38, or a pharmaceutically acceptable salt, solvate, tautomer, or stereoisomer thereof, wherein R¹ is hydrogen.

40. The compound of any one of claims 1-34, or a pharmaceutically acceptable salt, solvate, tautomer, or stereoisomer thereof, wherein X² is N.

41. The compound of any one of claims 1-40, or a pharmaceutically acceptable salt, solvate, tautomer, or stereoisomer thereof, wherein X² is CR².

42. The compound of any one of claims 1-41, or a pharmaceutically acceptable salt, solvate, tautomer, or stereoisomer thereof, wherein R² is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

43. The compound of any one of claims 1-42, or a pharmaceutically acceptable salt, solvate, tautomer, or stereoisomer thereof, wherein R² is hydrogen, deuterium, halogen, —OH, C₁-C₆alkyl, or C₁-C₆haloalkyl.

44. The compound of any one of claims 1-43, or a pharmaceutically acceptable salt, solvate, tautomer, or stereoisomer thereof, wherein R² is halogen.

45. The compound of any one of claims 1-40, or a pharmaceutically acceptable salt, solvate, tautomer, or stereoisomer thereof, wherein X² is N.

46. The compound of any one of claims 1-45, or a pharmaceutically acceptable salt, solvate, tautomer, or stereoisomer thereof, wherein X³ is CR³.

47. The compound of any one of claims 1-46, or a pharmaceutically acceptable salt, solvate, tautomer, or stereoisomer thereof, wherein R³ is hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl.

48. The compound of any one of claims 1-47, or a pharmaceutically acceptable salt, solvate, tautomer, or stereoisomer thereof, wherein R³ is hydrogen, deuterium, halogen, C₁-C₆alkyl, C₁-C₆haloalkyl, or C₁-C₆hydroxyalkyl, C₁-C₆heteroalkyl.

49. The compound of any one of claims 1-48, or a pharmaceutically acceptable salt, solvate, tautomer, or stereoisomer thereof, wherein R³ is hydrogen.

50. The compound of any one of claims 1-45, or a pharmaceutically acceptable salt, solvate, tautomer, or stereoisomer thereof, wherein X³ is N.

51. The compound of any one of claims 1-50, or a pharmaceutically acceptable salt, solvate, tautomer, or stereoisomer thereof, wherein X⁴ is CR⁴.

52. The compound of any one of claims 1-51, or a pharmaceutically acceptable salt, solvate, tautomer, or stereoisomer thereof, wherein R⁴ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl.

53. The compound of any one of claims 1-52, or a pharmaceutically acceptable salt, solvate, tautomer, or stereoisomer thereof, wherein R⁴ is hydrogen, deuterium, halogen, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

54. The compound of any one of claims 1-50, or a pharmaceutically acceptable salt, solvate, tautomer, or stereoisomer thereof, wherein X⁴ is N.

55. The compound of any one of claims 1-54, or a pharmaceutically acceptable salt, solvate, tautomer, or stereoisomer thereof, wherein R⁷ is halogen, —CN, —NO₂, —OH, —OR^{A}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl.

56. The compound of any one of claims 1-55, or a pharmaceutically acceptable salt, solvate, tautomer, or stereoisomer thereof, wherein R⁷ is hydrogen, deuterium, halogen, C₁-C₆alkyl, C₁-C₆haloalkyl, or C₁-C₆deuteroalkyl.

57. The compound of any one of claims 1-56, or a pharmaceutically acceptable salt, solvate, tautomer, or stereoisomer thereof, wherein R⁷ is C₁-C₆alkyl.

58. The compound of any one of claims 1-57, or a pharmaceutically acceptable salt, solvate, tautomer, or stereoisomer thereof, wherein R⁸ is hydrogen or C₁-C₆alkyl.

59. The compound of any one of claims 1-58, or a pharmaceutically acceptable salt, solvate, tautomer, or stereoisomer thereof, wherein R⁸ is hydrogen.

60. The compound of any one of claims 1-59, or a pharmaceutically acceptable salt, solvate, tautomer, or stereoisomer thereof, wherein Ring B is aryl or heteroaryl.

61. The compound of any one of claims 1-60, or a pharmaceutically acceptable salt, solvate, tautomer, or stereoisomer thereof, wherein Ring B is phenyl.

62. The compound of any one of claims 1-61, or a pharmaceutically acceptable salt, solvate, tautomer, or stereoisomer thereof, wherein each R⁹ is independently deuterium, halogen, C₁-C₆alkyl, C₁-C₆haloalkyl, C₁-C₆deuteroalkyl, cycloalkyl, or heterocycloalkyl.

63. The compound of any one of claims 1-62, or a pharmaceutically acceptable salt, solvate, tautomer, or stereoisomer thereof, wherein each R⁹ is independently halogen or C₁-C₆alkyl.

64. The compound of any one of claims 1-63, or a pharmaceutically acceptable salt, solvate, tautomer, or stereoisomer thereof, wherein m is 1-3.

65. A compound selected from the group consisting of: 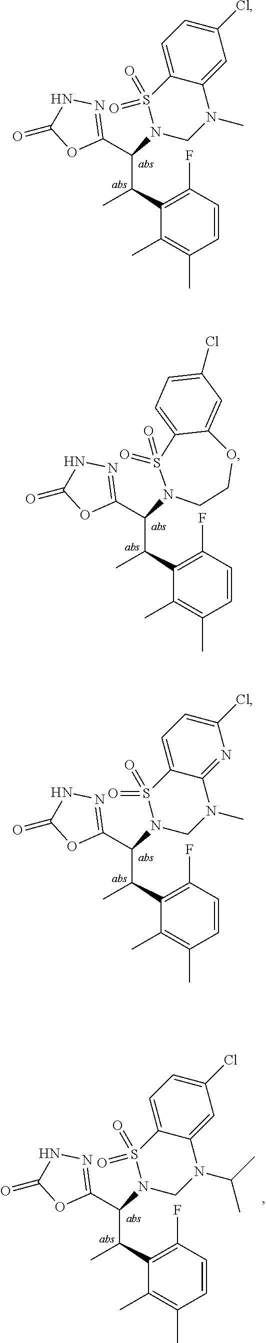 [SMILES: Cc1ccc(F)c([C@@H](C)[C@@H](c2n[nH]c(=O)o2)N2CN(C)c3cc(Cl)ccc3S2(=O)=O)c1C ; Cc1ccc(F)c([C@@H](C)[C@@H](c2n[nH]c(=O)o2)N2CCOc3cc(Cl)ccc3S2(=O)=O)c1C ; Cc1ccc(F)c([C@@H](C)[C@@H](c2n[nH]c(=O)o2)N2CN(C)c3nc(Cl)ccc3S2(=O)=O)c1C ; Cc1ccc(F)c([C@@H](C)[C@@H](c2n[nH]c(=O)o2)N2CN(C(C)C)c3cc(Cl)ccc3S2(=O)=O)c1C] 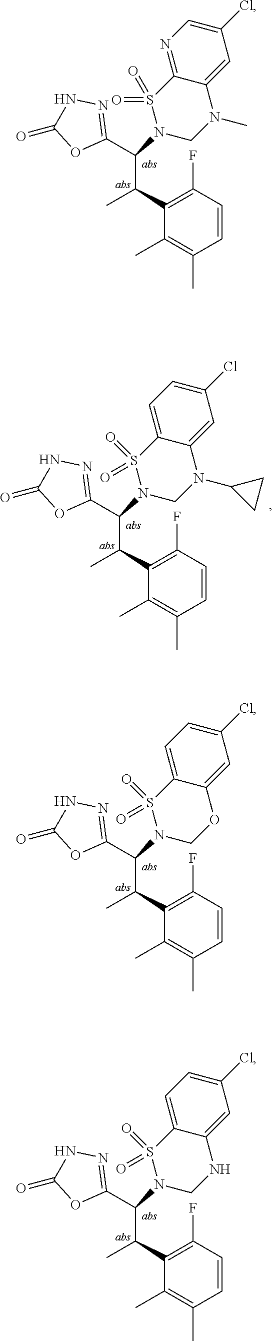 [SMILES: Cc1ccc(F)c([C@@H](C)[C@@H](c2n[nH]c(=O)o2)N2CN(C)c3cc(Cl)cnc3S2(=O)=O)c1C ; Cc1ccc(F)c([C@@H](C)[C@@H](c2n[nH]c(=O)o2)N2CN(C3CC3)c3cc(Cl)ccc3S2(=O)=O)c1C ; Cc1ccc(F)c([C@@H](C)[C@@H](c2n[nH]c(=O)o2)N2COc3cc(Cl)ccc3S2(=O)=O)c1C ; Cc1ccc(F)c([C@@H](C)[C@@H](c2n[nH]c(=O)o2)N2CNc3cc(Cl)ccc3S2(=O)=O)c1C] 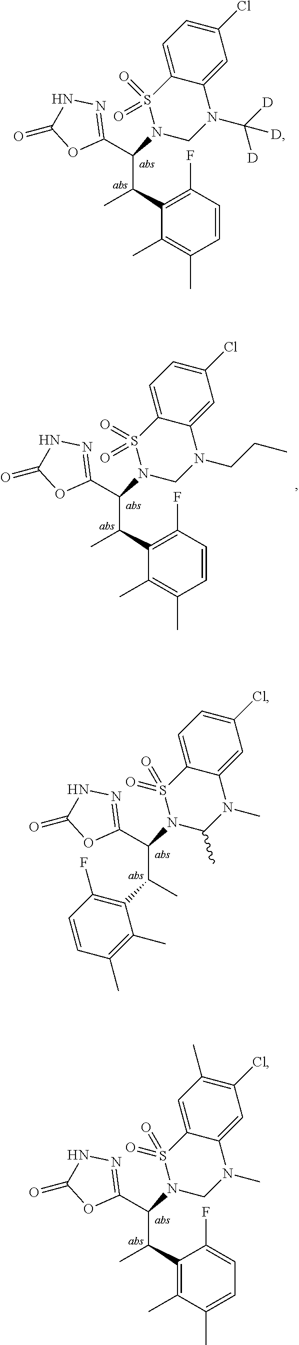 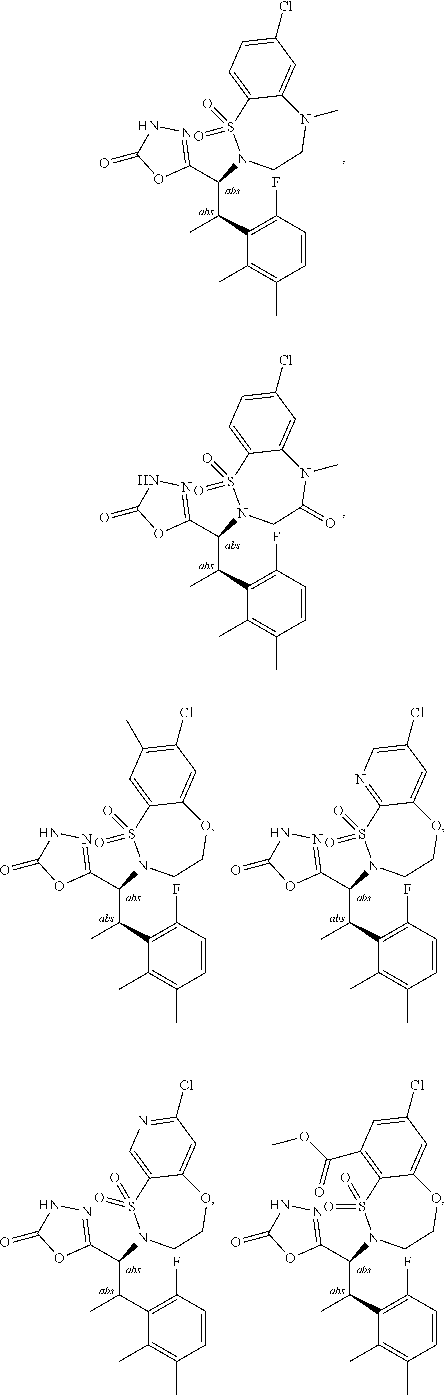 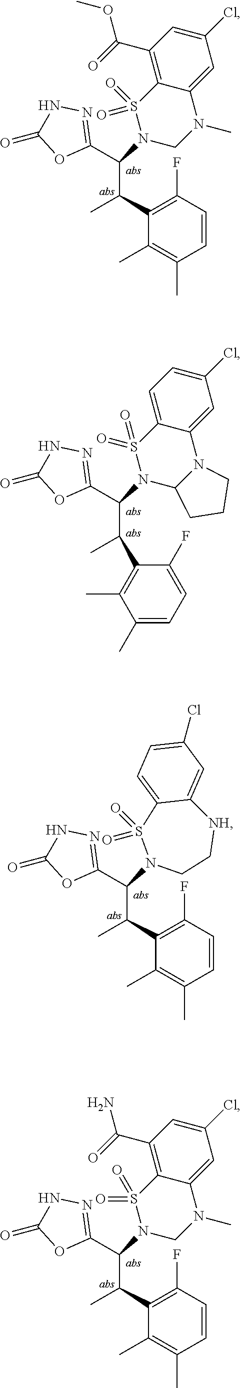 [SMILES: COC(=O)c1cc(Cl)cc2c1S(=O)(=O)N([C@H](c1n[nH]c(=O)o1)[C@H](C)c1c(F)ccc(C)c1C)CN2C ; Cc1ccc(F)c([C@@H](C)[C@@H](c2n[nH]c(=O)o2)N2C3CCCN3c3cc(Cl)ccc3S2(=O)=O)c1C ; Cc1ccc(F)c([C@@H](C)[C@@H](c2n[nH]c(=O)o2)N2CCNc3cc(Cl)ccc3S2(=O)=O)c1C ; Cc1ccc(F)c([C@@H](C)[C@@H](c2n[nH]c(=O)o2)N2CN(C)c3cc(Cl)cc(C(N)=O)c3S2(=O)=O)c1C] 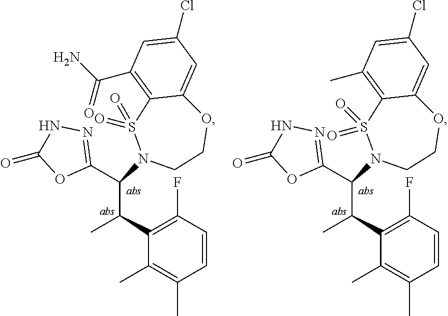 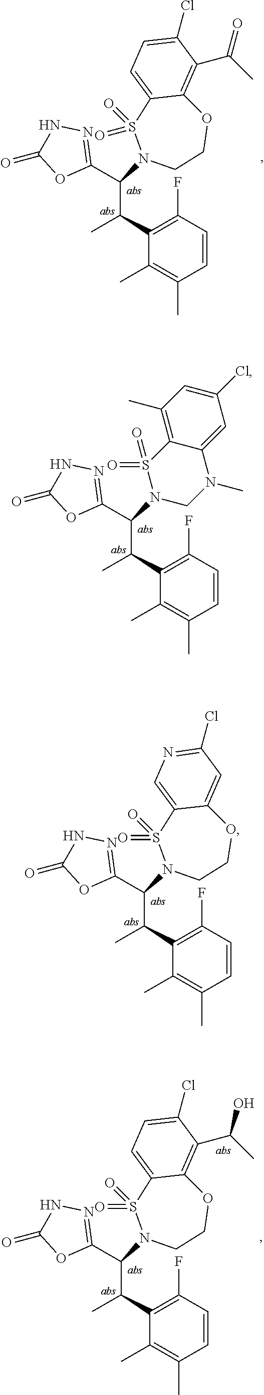 [SMILES: CC(=O)c1c(Cl)ccc2c1OCCN([C@H](c1n[nH]c(=O)o1)[C@H](C)c1c(F)ccc(C)c1C)S2(=O)=O ; Cc1ccc(F)c([C@@H](C)[C@@H](c2n[nH]c(=O)o2)N2CN(C)c3cc(Cl)cc(C)c3S2(=O)=O)c1C ; Cc1ccc(F)c([C@@H](C)[C@@H](c2n[nH]c(=O)o2)N2CCOc3cc(Cl)ncc3S2(=O)=O)c1C ; Cc1ccc(F)c([C@@H](C)[C@@H](c2n[nH]c(=O)o2)N2CCOc3c(ccc(Cl)c3[C@H](C)O)S2(=O)=O)c1C] 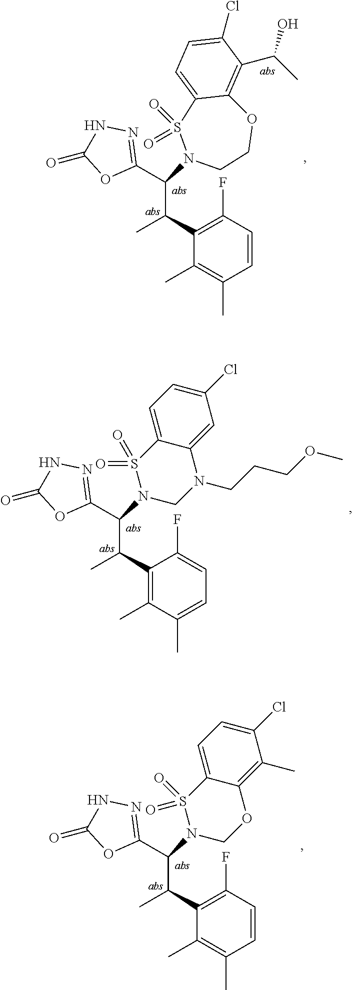 [SMILES: Cc1ccc(F)c([C@@H](C)[C@@H](c2n[nH]c(=O)o2)N2CCOc3c(ccc(Cl)c3[C@@H](C)O)S2(=O)=O)c1C ; COCCCN1CN([C@H](c2n[nH]c(=O)o2)[C@H](C)c2c(F)ccc(C)c2C)S(=O)(=O)c2ccc(Cl)cc21 ; Cc1ccc(F)c([C@@H](C)[C@@H](c2n[nH]c(=O)o2)N2COc3c(ccc(Cl)c3C)S2(=O)=O)c1C] 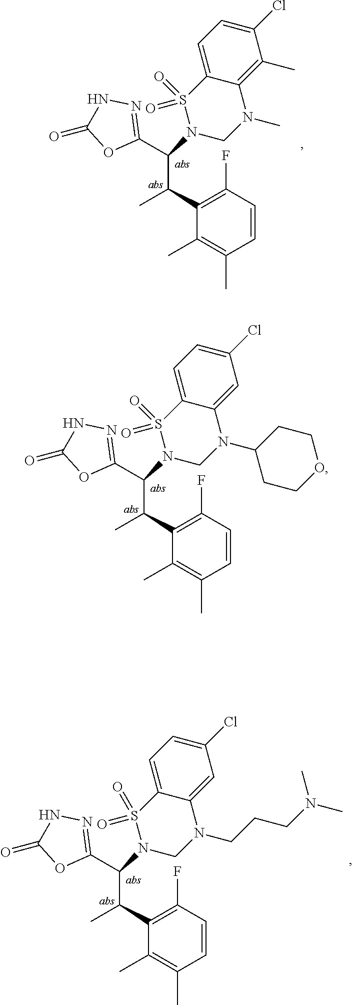 [SMILES: Cc1ccc(F)c([C@@H](C)[C@@H](c2n[nH]c(=O)o2)N2CN(C)c3c(ccc(Cl)c3C)S2(=O)=O)c1C ; Cc1ccc(F)c([C@@H](C)[C@@H](c2n[nH]c(=O)o2)N2CN(C3CCOCC3)c3cc(Cl)ccc3S2(=O)=O)c1C ; Cc1ccc(F)c([C@@H](C)[C@@H](c2n[nH]c(=O)o2)N2CN(CCCN(C)C)c3cc(Cl)ccc3S2(=O)=O)c1C] 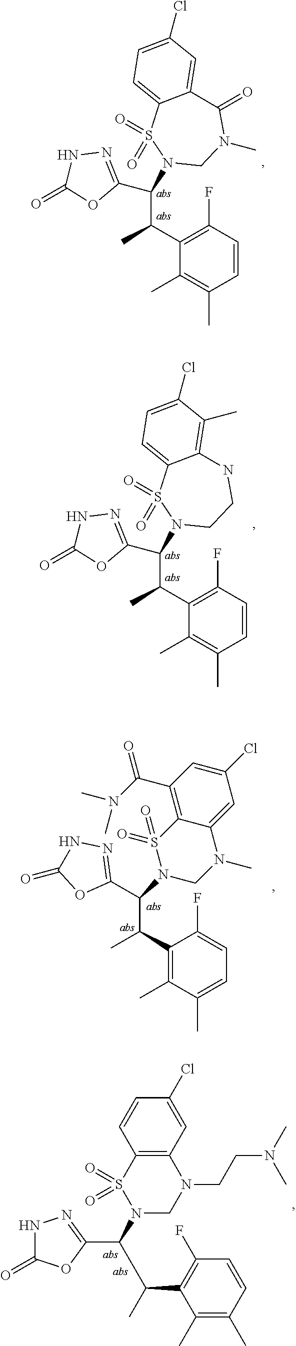 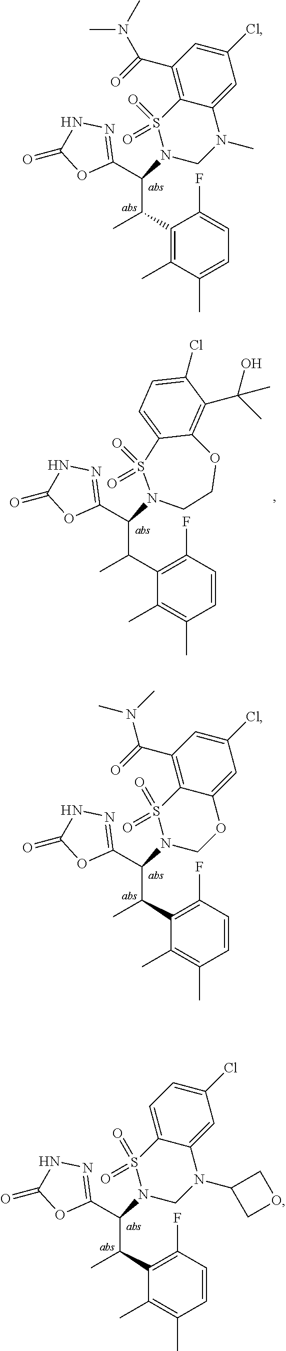 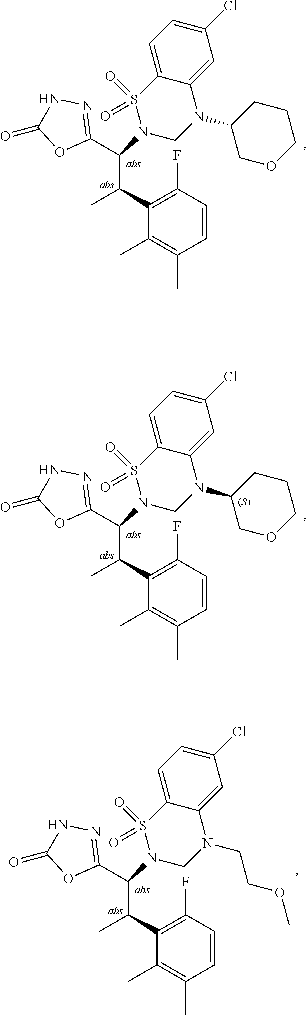 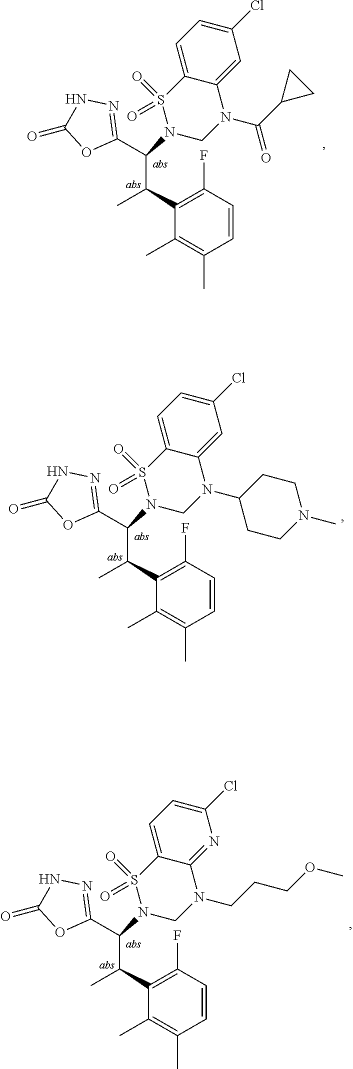 [SMILES: Cc1ccc(F)c([C@@H](C)[C@@H](c2n[nH]c(=O)o2)N2CN(C(=O)C3CC3)c3cc(Cl)ccc3S2(=O)=O)c1C ; Cc1ccc(F)c([C@@H](C)[C@@H](c2n[nH]c(=O)o2)N2CN(C3CCN(C)CC3)c3cc(Cl)ccc3S2(=O)=O)c1C ; COCCCN1CN([C@H](c2n[nH]c(=O)o2)[C@H](C)c2c(F)ccc(C)c2C)S(=O)(=O)c2ccc(Cl)nc21] 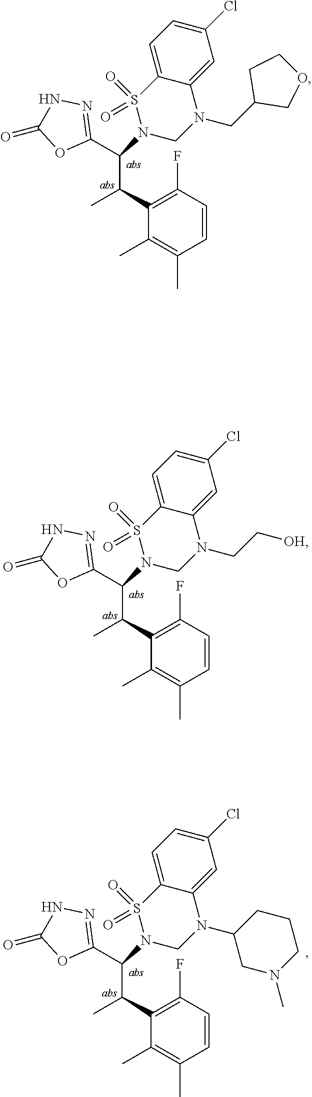 [SMILES: Cc1ccc(F)c([C@@H](C)[C@@H](c2n[nH]c(=O)o2)N2CN(CC3CCOC3)c3cc(Cl)ccc3S2(=O)=O)c1C ; Cc1ccc(F)c([C@@H](C)[C@@H](c2n[nH]c(=O)o2)N2CN(CCO)c3cc(Cl)ccc3S2(=O)=O)c1C ; Cc1ccc(F)c([C@@H](C)[C@@H](c2n[nH]c(=O)o2)N2CN(C3CCCN(C)C3)c3cc(Cl)ccc3S2(=O)=O)c1C] 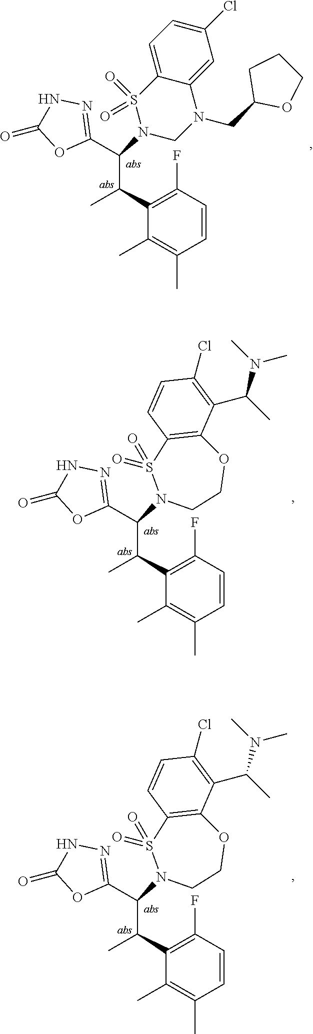 [SMILES: Cc1ccc(F)c([C@@H](C)[C@@H](c2n[nH]c(=O)o2)N2CN(C[C@H]3CCCO3)c3cc(Cl)ccc3S2(=O)=O)c1C ; Cc1ccc(F)c([C@@H](C)[C@@H](c2n[nH]c(=O)o2)N2CCOc3c(ccc(Cl)c3[C@H](C)N(C)C)S2(=O)=O)c1C ; Cc1ccc(F)c([C@@H](C)[C@@H](c2n[nH]c(=O)o2)N2CCOc3c(ccc(Cl)c3[C@@H](C)N(C)C)S2(=O)=O)c1C] 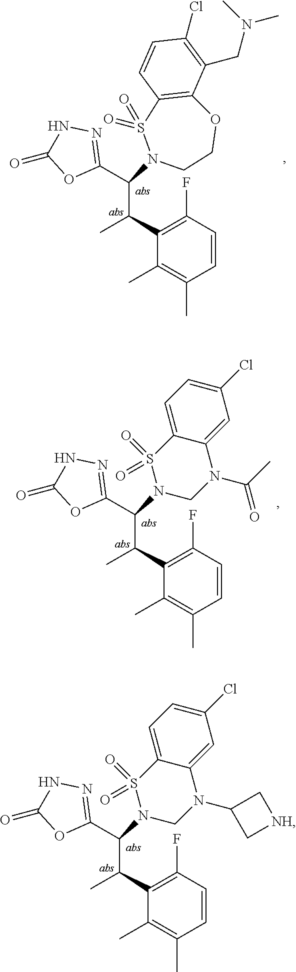 [SMILES: Cc1ccc(F)c([C@@H](C)[C@@H](c2n[nH]c(=O)o2)N2CCOc3c(ccc(Cl)c3CN(C)C)S2(=O)=O)c1C ; CC(=O)N1CN([C@H](c2n[nH]c(=O)o2)[C@H](C)c2c(F)ccc(C)c2C)S(=O)(=O)c2ccc(Cl)cc21 ; Cc1ccc(F)c([C@@H](C)[C@@H](c2n[nH]c(=O)o2)N2CN(C3CNC3)c3cc(Cl)ccc3S2(=O)=O)c1C] 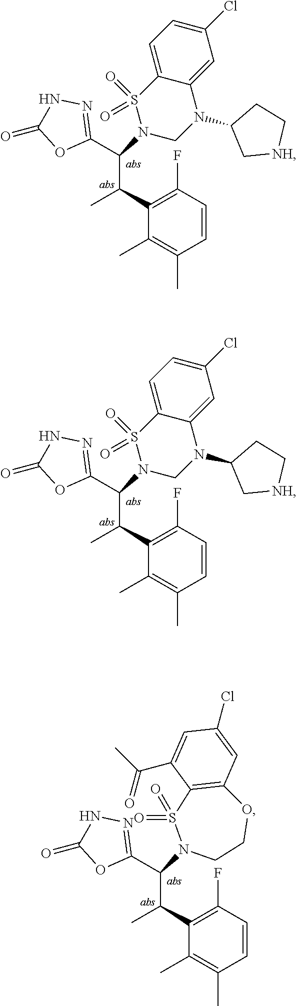 [SMILES: Cc1ccc(F)c([C@@H](C)[C@@H](c2n[nH]c(=O)o2)N2CN([C@@H]3CCNC3)c3cc(Cl)ccc3S2(=O)=O)c1C ; Cc1ccc(F)c([C@@H](C)[C@@H](c2n[nH]c(=O)o2)N2CN([C@H]3CCNC3)c3cc(Cl)ccc3S2(=O)=O)c1C ; CC(=O)c1cc(Cl)cc2c1S(=O)(=O)N([C@H](c1n[nH]c(=O)o1)[C@H](C)c1c(F)ccc(C)c1C)CCO2] 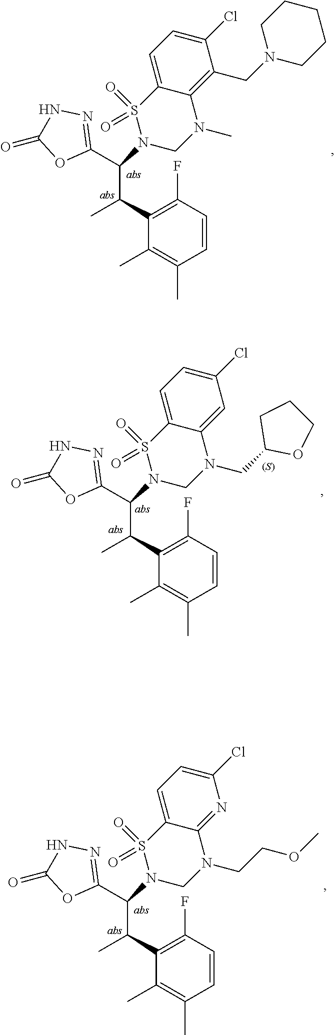 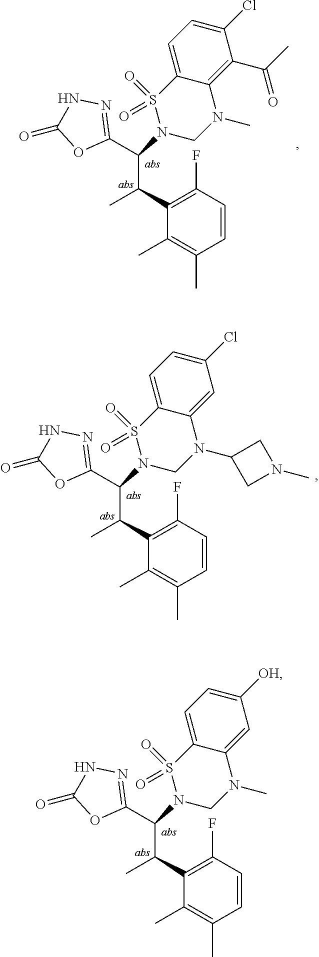 [SMILES: CC(=O)c1c(Cl)ccc2c1N(C)CN([C@H](c1n[nH]c(=O)o1)[C@H](C)c1c(F)ccc(C)c1C)S2(=O)=O ; Cc1ccc(F)c([C@@H](C)[C@@H](c2n[nH]c(=O)o2)N2CN(C3CN(C)C3)c3cc(Cl)ccc3S2(=O)=O)c1C ; Cc1ccc(F)c([C@@H](C)[C@@H](c2n[nH]c(=O)o2)N2CN(C)c3cc(O)ccc3S2(=O)=O)c1C] 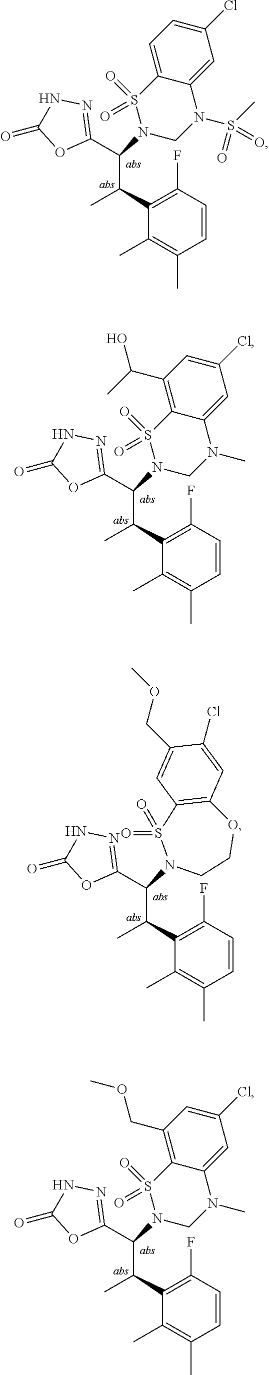 [SMILES: Cc1ccc(F)c([C@@H](C)[C@@H](c2n[nH]c(=O)o2)N2CN(S(C)(=O)=O)c3cc(Cl)ccc3S2(=O)=O)c1C ; Cc1ccc(F)c([C@@H](C)[C@@H](c2n[nH]c(=O)o2)N2CN(C)c3cc(Cl)cc(C(C)O)c3S2(=O)=O)c1C ; COCc1cc2c(cc1Cl)OCCN([C@H](c1n[nH]c(=O)o1)[C@H](C)c1c(F)ccc(C)c1C)S2(=O)=O ; COCc1cc(Cl)cc2c1S(=O)(=O)N([C@H](c1n[nH]c(=O)o1)[C@H](C)c1c(F)ccc(C)c1C)CN2C] 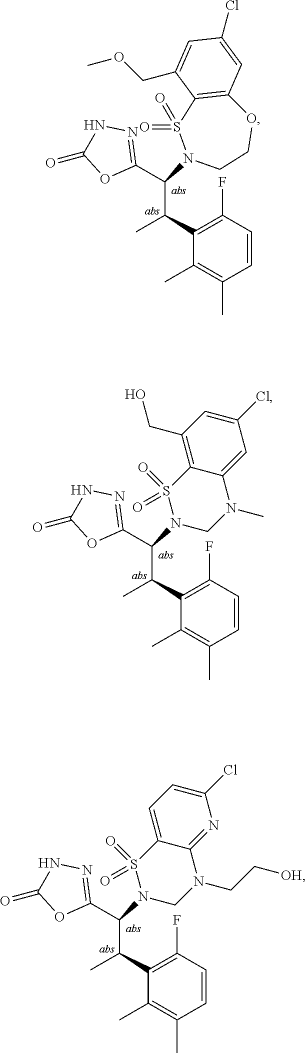 [SMILES: COCc1cc(Cl)cc2c1S(=O)(=O)N([C@H](c1n[nH]c(=O)o1)[C@H](C)c1c(F)ccc(C)c1C)CCO2 ; Cc1ccc(F)c([C@@H](C)[C@@H](c2n[nH]c(=O)o2)N2CN(C)c3cc(Cl)cc(CO)c3S2(=O)=O)c1C ; Cc1ccc(F)c([C@@H](C)[C@@H](c2n[nH]c(=O)o2)N2CN(CCO)c3nc(Cl)ccc3S2(=O)=O)c1C] 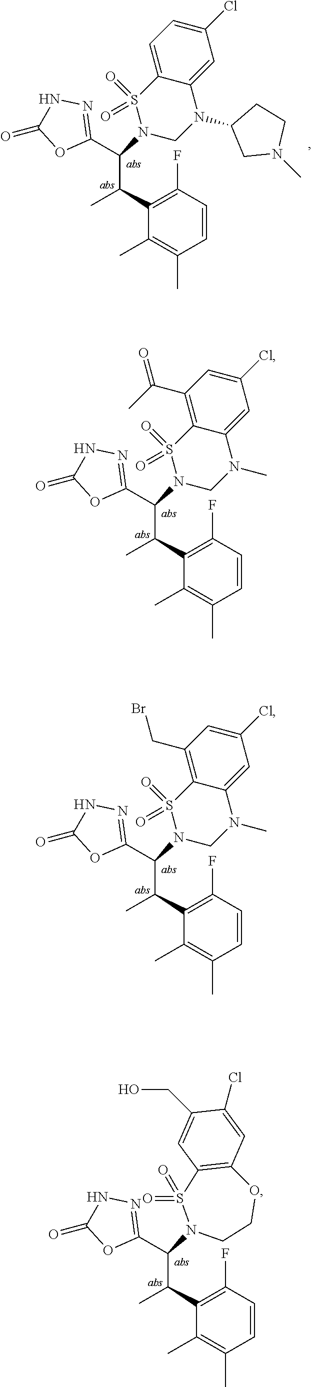 [SMILES: Cc1ccc(F)c([C@@H](C)[C@@H](c2n[nH]c(=O)o2)N2CN([C@@H]3CCN(C)C3)c3cc(Cl)ccc3S2(=O)=O)c1C ; CC(=O)c1cc(Cl)cc2c1S(=O)(=O)N([C@H](c1n[nH]c(=O)o1)[C@H](C)c1c(F)ccc(C)c1C)CN2C ; Cc1ccc(F)c([C@@H](C)[C@@H](c2n[nH]c(=O)o2)N2CN(C)c3cc(Cl)cc(CBr)c3S2(=O)=O)c1C ; Cc1ccc(F)c([C@@H](C)[C@@H](c2n[nH]c(=O)o2)N2CCOc3cc(Cl)c(CO)cc3S2(=O)=O)c1C] 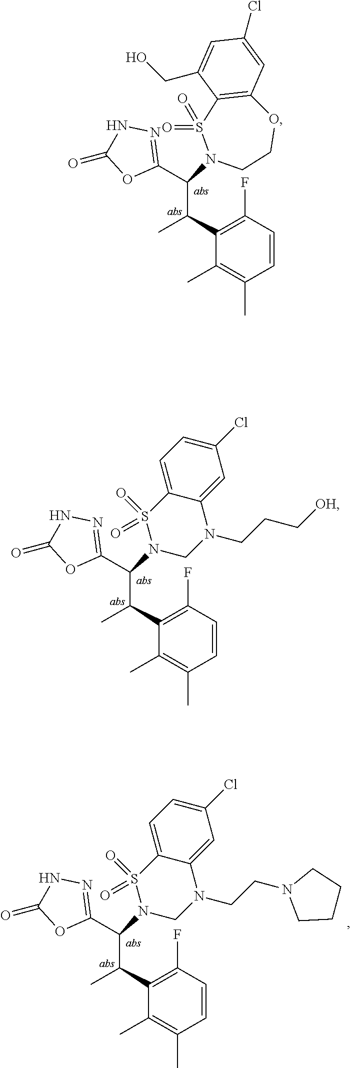 [SMILES: Cc1ccc(F)c([C@@H](C)[C@@H](c2n[nH]c(=O)o2)N2CCOc3cc(Cl)cc(CO)c3S2(=O)=O)c1C ; Cc1ccc(F)c([C@@H](C)[C@@H](c2n[nH]c(=O)o2)N2CN(CCCO)c3cc(Cl)ccc3S2(=O)=O)c1C ; Cc1ccc(F)c([C@@H](C)[C@@H](c2n[nH]c(=O)o2)N2CN(CCN3CCCC3)c3cc(Cl)ccc3S2(=O)=O)c1C] 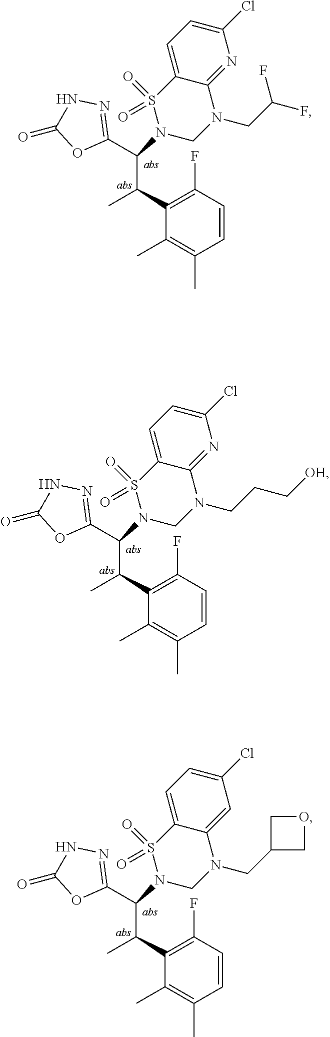 [SMILES: Cc1ccc(F)c([C@@H](C)[C@@H](c2n[nH]c(=O)o2)N2CN(CC(F)F)c3nc(Cl)ccc3S2(=O)=O)c1C ; Cc1ccc(F)c([C@@H](C)[C@@H](c2n[nH]c(=O)o2)N2CN(CCCO)c3nc(Cl)ccc3S2(=O)=O)c1C ; Cc1ccc(F)c([C@@H](C)[C@@H](c2n[nH]c(=O)o2)N2CN(CC3COC3)c3cc(Cl)ccc3S2(=O)=O)c1C] 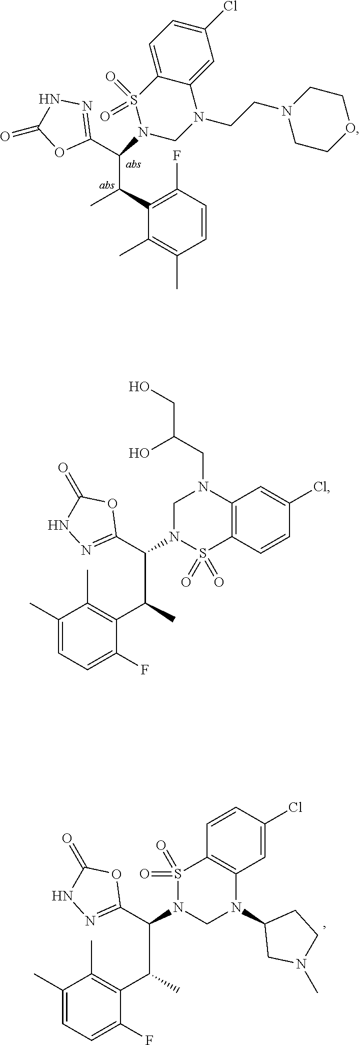 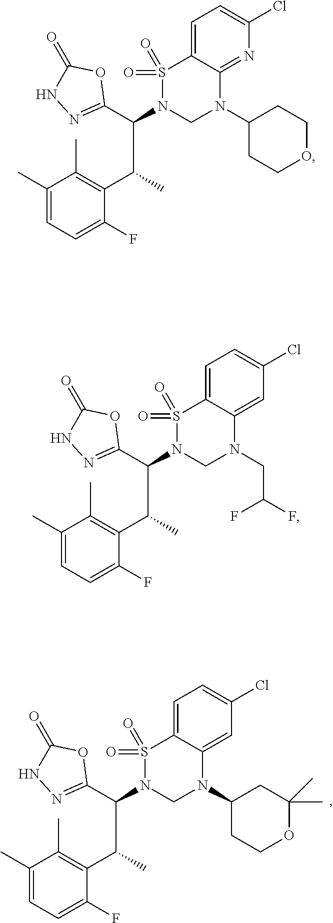 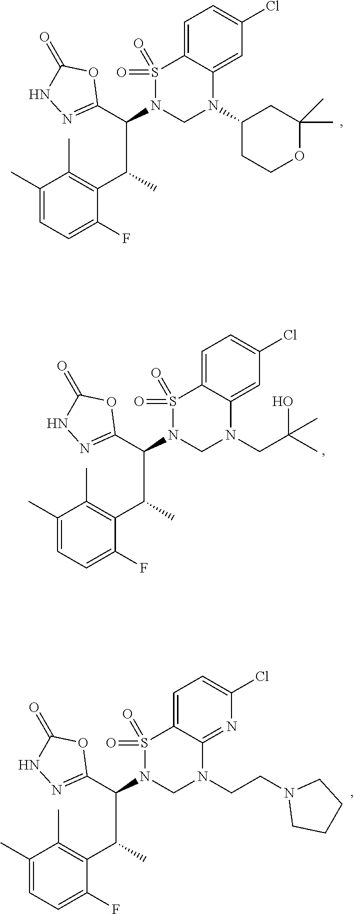 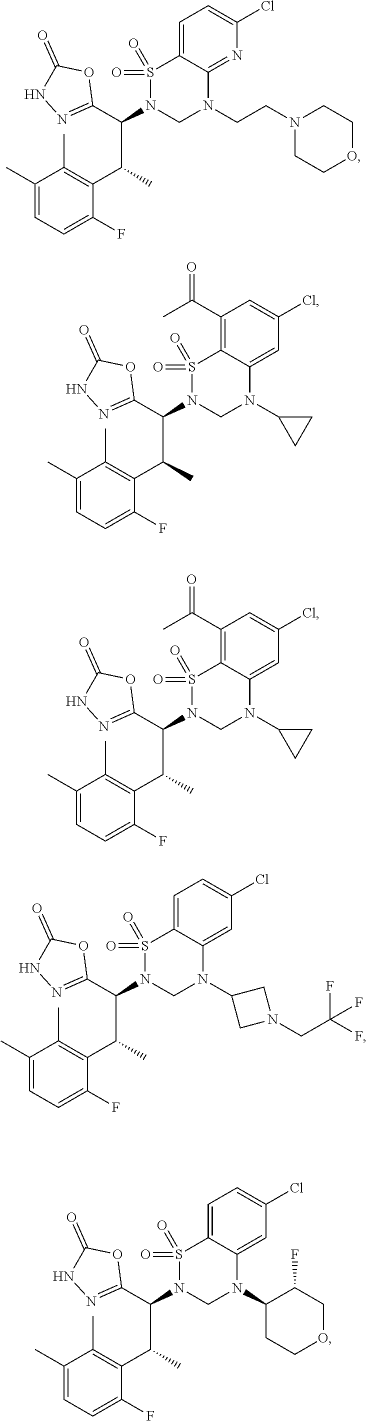 [SMILES: Cc1ccc(F)c([C@@H](C)[C@@H](c2n[nH]c(=O)o2)N2CN(CCN3CCOCC3)c3nc(Cl)ccc3S2(=O)=O)c1C ; CC(=O)c1cc(Cl)cc2c1S(=O)(=O)N([C@H](c1n[nH]c(=O)o1)[C@@H](C)c1c(F)ccc(C)c1C)CN2C1CC1 ; CC(=O)c1cc(Cl)cc2c1S(=O)(=O)N([C@H](c1n[nH]c(=O)o1)[C@H](C)c1c(F)ccc(C)c1C)CN2C1CC1 ; Cc1ccc(F)c([C@@H](C)[C@@H](c2n[nH]c(=O)o2)N2CN(C3CN(CC(F)(F)F)C3)c3cc(Cl)ccc3S2(=O)=O)c1C ; Cc1ccc(F)c([C@@H](C)[C@@H](c2n[nH]c(=O)o2)N2CN([C@@H]3CCOC[C@H]3F)c3cc(Cl)ccc3S2(=O)=O)c1C] 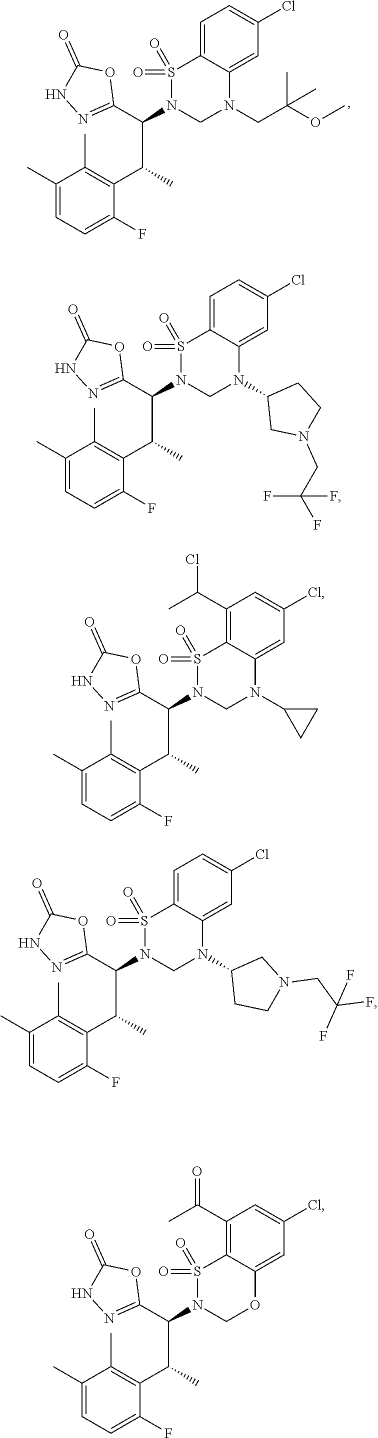 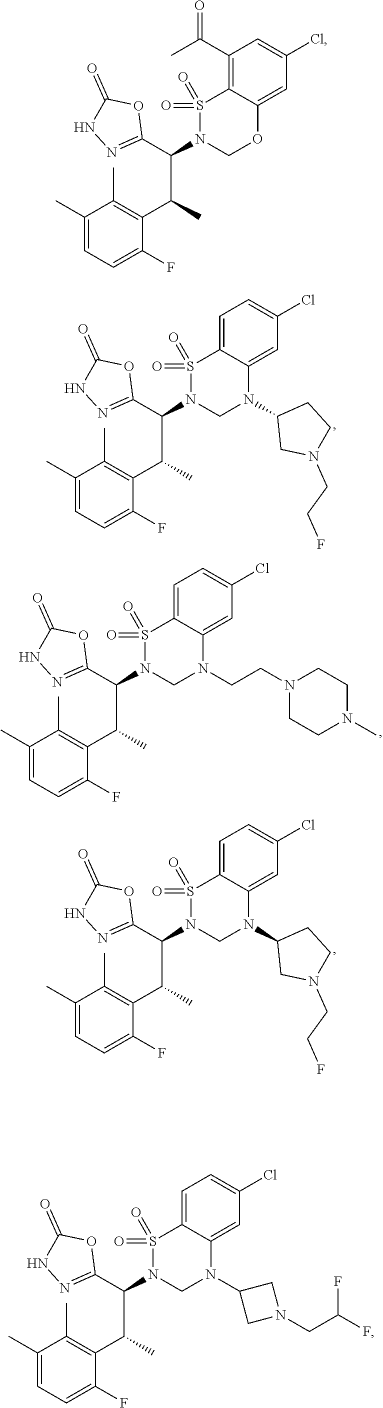 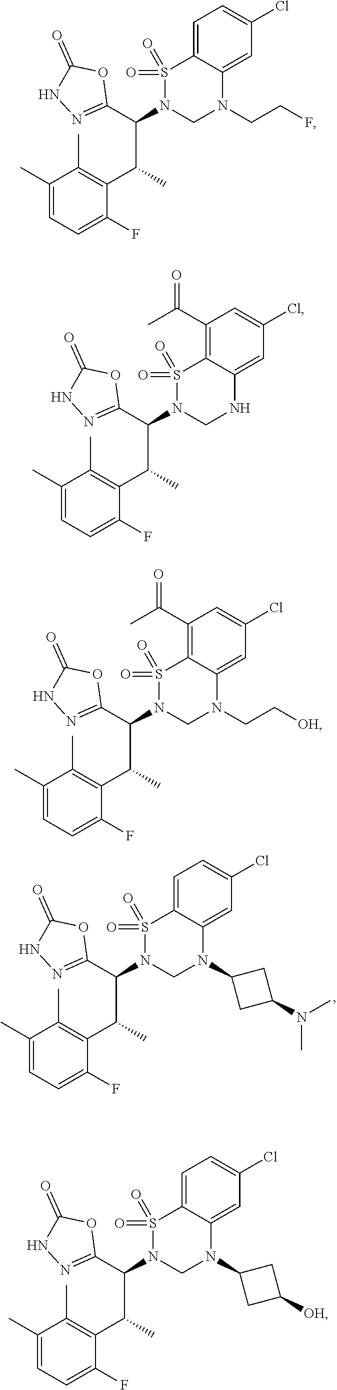 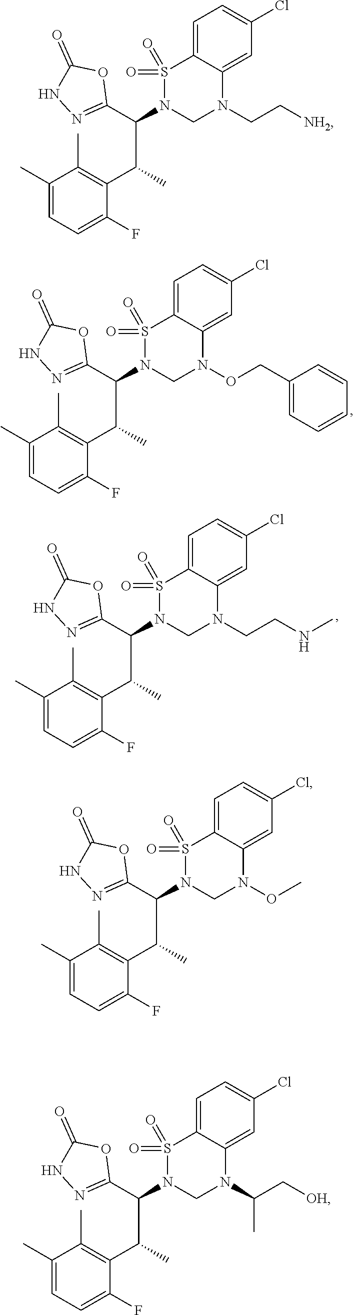 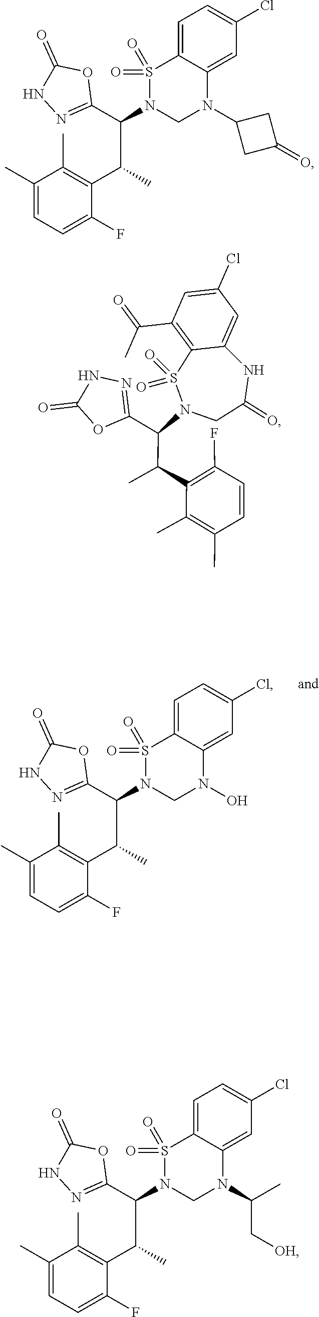 [SMILES: Cc1ccc(F)c([C@@H](C)[C@@H](c2n[nH]c(=O)o2)N2CN(C3CC(=O)C3)c3cc(Cl)ccc3S2(=O)=O)c1C ; CC(=O)c1cc(Cl)cc2c1S(=O)(=O)N([C@H](c1n[nH]c(=O)o1)[C@H](C)c1c(F)ccc(C)c1C)CC(=O)N2 ; Cc1ccc(F)c([C@@H](C)[C@@H](c2n[nH]c(=O)o2)N2CN(O)c3cc(Cl)ccc3S2(=O)=O)c1C ; Cc1ccc(F)c([C@@H](C)[C@@H](c2n[nH]c(=O)o2)N2CN([C@@H](C)CO)c3cc(Cl)ccc3S2(=O)=O)c1C]
or a pharmaceutically acceptable salt, solvate, tautomer, or stereoisomer thereof.

66. A pharmaceutical composition comprising a compound of any one of claims 1-65, or a pharmaceutically acceptable salt, solvate, tautomer, or stereoisomer thereof, and a pharmaceutically acceptable excipient.

67. A method of treating cancer in a subject, comprising administering to the subject a compound of any one of claims 1-65, or a pharmaceutically acceptable salt, solvate, tautomer, or stereoisomer thereof, or a pharmaceutical composition of claim 66.

68. A method of inhibiting ribonucleotide reductase in a subject, comprising administering to the subject a compound of any one of claims 1-65, or a pharmaceutically acceptable salt, solvate, tautomer, or stereoisomer thereof, or a pharmaceutical composition of claim 66.

69. The method of claim 68, wherein the inhibition of ribonucleotide reductase occurs in a tumor cell in the subject in need thereof.

70. A method for treating a tumor or tumor cells in a subject, the method comprising administering a compound of any one of claims 1-65, or a pharmaceutically acceptable salt, solvate, tautomer, or stereoisomer thereof, or the pharmaceutical composition of claim 66, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

71. A method of treating an ecDNA-associated tumor or tumor cells comprising administering a compound of any one of claims 1-65, or a pharmaceutically acceptable salt, solvate, tautomer, or stereoisomer thereof, or the pharmaceutical composition of claim 66, to a subject identified as having a tumor or tumor cells having ecDNA, wherein growth or size of the tumor or growth or number of the tumor cells is decreased as a result of treatment.

72. The method of claim 71, wherein the method further comprises administering a cancer-targeted therapeutic agent.

73. The method of claim 72, wherein the cancer-targeted therapeutic agent inhibits a gene or gene product comprised on ecDNA in the tumor or tumor cells.

74. A method for treating a tumor or tumor cells in a subject, the method comprising administering a compound of any one of claims 1-65, or a pharmaceutically acceptable salt, solvate, tautomer, or stereoisomer thereof, or the pharmaceutical composition of claim 66, in an amount sufficient to induce replication stress in the tumor or tumor cells, wherein the tumor or tumor cells comprises ecDNA or have an ecDNA signature; and wherein growth or size of the tumor or growth or number of tumor cells is reduced.

75. A method of treating an ecDNA-associated tumor or tumor cells comprising administering a compound of any one of claims 1-65, or a pharmaceutically acceptable salt, solvate, tautomer, or stereoisomer thereof, or the pharmaceutical composition of claim 66, to a subject identified as having a tumor or tumor cells having a focal amplification of an oncogene, wherein growth or size of the tumor or growth or number of the tumor cells is decreased as a result of treatment.

76. The method of claim 75, further comprises administering a cancer-targeted therapeutic agent, wherein the target of the therapeutic agent is a protein encoded by the oncogene.

77. The method of claim 75 or 76, wherein the focal amplification is present on ecDNA.
